SEQUENCE LISTING (391 sequences)

SEQ ID NO: 1 - DNA, 126 bp, synthetic construct
  misc_feature 1..126: note = synthetic construct
  source 1..126: mol_type = other DNA
gattattatactatcaaagatttttctaatctgtctagtatcaaaatagattaagaatttacattcctctaacttatcaagtttttaccctactccatagttctctaacttgccctttccttacaa

SEQ ID NO: 2 - DNA, 499 bp, White clover cryptic virus 1
  source 1..499: mol_type = other DNA
gttttaacttaagcttttcgtaatcaaaatagaacgcgaatctcaactgctctacctcaactttttacctactctaccgctaacttaccgccctacccacaagatgaatcaagacactcctctcgccaatctcaatggccccgaagttccatccggcaacgtccctcccgccaaccctcctggtcgaacaaacgtcgcccctcccgcccaaggtgctgttcaacagcctcctgctccggctgctcgtcgtgcccgcaacccacacggccctgtccctcctgctggaccatcccgttctgctggtgcccctgctatgcttgaactttccgctgcctaccctatgtacaccgaacaacgtcgtgcacccaactactatgtgcctgacgcacaacttctcttccacaccctcggtgtctgcgaccaactgatgaccacaactgaccgcttcctccgctccatgccttcatggttgccaatcgtctctcagctttacgtctct

SEQ ID NO: 3 - DNA, 500 bp, Vicia cryptic virus
  source 1..500: mol_type = other DNA
gatcaaaagagtaacagtccttgtaccaacttttcacaatcaaaatagaacgcgaatcttgactgctctacctcaactttttacttactttaacgttgtttatcaaccgtccctctcaatggaagctcatactcccgctgctgatgtcaatggacccaatatcccctccggtgctgtctctcagccagaaatcgctccacacaatcaagctgcccccaacgtctctggtgctgctcaggctataatagcccccacccctgctcgtaagcgcacgcctcacggacccattcctgctgccacctcatctggcaacacctctgctcctgctctcctggaattagctgctgcttatcccatgtacaccgagcaacgccgttcattcaactttttcatcccagactcccaaatgatgtttcactctttaggtatctgcgactcaatgatgaactccaccgatcgttttcttcgatcctcacccgcttggctccccatcgtttcac

SEQ ID NO: 4 - DNA, 500 bp, unidentified
  misc_feature 1..500: note = unclassified Partitiviridae
  source 1..500: mol_type = other DNA
agaaaaattttagaacctactctaattgatttttctaagctttatttcttttcctctgaaaaccaactttcaacataatggctcatcgtactcccactaatgccccggctcttcctcaagttgatgggatcaaccccaaccttccccccaacgacccaggaaccgtcgccgccgcccctaatgctaggaacttgcatctcgaaagagaacaagaagctagacaagaccgcgccgccgccgccttcgtccccaacactcgctttgcggtgagaagaccgacgattcgcccctccccggcccactctgcccctgacctagagactgctctctttgattcagccgtcaaccacccccccaccccggtctcctatccgtctacctcatcctacatccccaacttcacttccgccttctactatctcaacaagatggactccttgatggtccaaactctcaactggacaaacaactgctctggatgggttcccccatactcccag

SEQ ID NO: 5 - DNA, 500 bp, unidentified
  misc_feature 1..500: note = unclassified Partitiviridae
  source 1..500: mol_type = other DNA
gataatgatcctgggaaagatcgttattattttactagtgtaaatttcttaaagagcttttcttcaaggtaattttcactttctcactttccccttagatgttatctaagcttgccttgttataataatttataactctactagtatgtttactatcttactttcgtagatatggctagcccacgtgactctatgatgacgactgctcaagacgctaagaaacgtaaagtttctcatattttgagtccgattcgaatgatcgagcctgctgctcaatccctcgcaatcatcccagttgtccctcaagtccaaatggatgacgctgaagctatggctgaattgctgtatcgtgaacgcgcccgcattgaacttgatattaatcctgcttatatcagtagattggaatcccgtcgagagattggtttctatcctcctgttgcaagtaagaacgttccgttcactactttcaacatcgatgtcgctgaattcagacgctgcat

SEQ ID NO: 6 - DNA, 500 bp, unidentified
  misc_feature 1..500: note = unclassified Partitiviridae
  source 1..500: mol_type = other DNA
gataatgatccaccgaaaggtaatcattgtctctttagttaggaattaaacttcaaatttcatttaaacttatgaacctacttagaagtttcttcccctatcttactaaagacctgtttgtttgatatgtttcagtgagtacctaaactcatacatcttacatccgctactaacgagcaatcaccatcccatggcgactgacaatagcgacgctaagactgagcaatcaaaagtcacggagagcaaaggttcttcaccaacgaagagcttacccgctcatgctgctgacgagaaagccgctgttggtgctaccttcaagctgcaatccagacaacccactgagccacccgctataccaagcgtattcgctcaatcacaatcacttaatgattctgtgggaaacccagactttatagcgcaactgcgagatgaaggcatgtacggcctgtttctgccaagagatcgagcccgtcgcacgcctacaccagtaactctgcggc

SEQ ID NO: 7 - DNA, 500 bp, unidentified
  misc_feature 1..500: note = unclassified Partitiviridae
  source 1..500: mol_type = other DNA
gataatgtttcattaggaaacattatcccggattagccggtagcttttgtgtatagatttatgttaggaaacatgtctttgtctcactgaccacacaagtataccactaaccaagtttctttattgtttcaggattaattaaccagtgaattattcaccttacaatcgccgctaaccaagcacattaccactttgtgatcaaccgacgatggcttttgaatccaaaacatccgcttccgaaagcactgtgaacaaagatgcagctcccgctccgtcctccgcaagtgtcaaatctccagtcaagccctccagctctggactcggccgcgccaaaacagaaacgccccacctcccggcatctaactatgagatcccgcgttacacacgcgatactcagccggttgttggaaacaagaattacgaattctctctccgcatggatggacgccttggccactcgatggaccgctccaaaacggcccgccctgaagtcaactgct

SEQ ID NO: 8 - DNA, 500 bp, unidentified
  misc_feature 1..500: note = unclassified Partitiviridae
  source 1..500: mol_type = other DNA
gataatgatcgtttatcgatcattatcataggttaggtctagaggtctagatagattagctttaggttagtcataacgactctctagtacacacactaaccaacttgtgtttttacattgcaggtttaatttcgtgaattaacgtaaagctttttcacccttacctcgcgatggatacagcacaaacagacgctgacaaagctgctggaaaacgtggagcaactgtgccgcccgaaggtgagcccgctgcaaaaaccatgagattttccgacgctattgtcccagctggtccgagcaaggatgcttctactgttgcaaccgccgccctgcccaaagatcttatacagtggccgattgaacggctagctacacaggtcaaccaagtctttctcgacaccaccatccagaatggagaaggtggatatgaagtccctgacgatcaaatcgtcccaacacttcgtgtttacgagatcaacgtgaccaatctgcggaacgctttt

SEQ ID NO: 9 - DNA, 500 bp, unidentified
  misc_feature 1..500: note = unclassified Partitiviridae
  source 1..500: mol_type = other DNA
gataatgatccaccgaaaaggttcattatctctttagttaggatttaacttctaagtttcacttcatacttataaacttactaagaagttgctatacatcctacactaacgaattcctgtttgatttgtttcagtgagtaactaaactcatacatcttacgaccgtagctaacgagcctacccaacaccatggataaggatgctgccgccgcccagactgagcaatcaaagattacggagagccacggatcttcacctgagaagaatgccgccgctcacactgctgatcagaaagccgctgttggatcaacctttcagctgcaatcccggaagcccacagagccacccactgtaccaagtgcgttcgctcaatctacggcacttaatgtgtctgtgggtaatgcagactatatagctgaacaaagagatgaaggaaaatatggcttgtttctaccaagagatagagcccaccgtgaacccgaacctgtcaccatgatagt

SEQ ID NO: 10 - DNA, 499 bp, unidentified
  misc_feature 1..499: note = unclassified Partitiviridae
  source 1..499: mol_type = other DNA
gataatgatccagggaaagatcattattatctttaagtgattaactttctgtaaaatctacattacaggtaattattcactccctaccttccccgtagttctgtacgaagcttagcgttttataaaattttataaagctatctgcattttaactatcctagttccgcaggtatggctagctccaaacgaactccgccgcttgttgcacaacaaccgaagaaacgccgtttcgaaacggtcttcgatcttggacccagcttaggatctacatccctcgctatggtacatgtgccacaaccgactccaatgctaacggatgaagctgaacaaatggctgaattgctatttcgcgaacgctcccgaattgaattggatattaatcctgcttatgtcagtgaactcgaacttcgtcgtgaagtcggattttctccgatgatcgctaataaaactttagctttcactacttacgacctacgagtcgttgaattcagacgctgca

SEQ ID NO: 11 - DNA, 500 bp, Beet cryptic virus 1
  source 1..500: mol_type = other DNA
gatcaaaagatcaacagctcttgttcaaagctttctgtaatcaaaatagaacacagatctcagctgctctgactcaacttgttacctactctaagatactcaatatcgcccctctcgataagatggaaaacaacactcctctggctaaccctagcggccccaatgtaccctccgctgctgccccccccacccccgcccctccggcgatacctcaggctacccccactcccggtgctgtctctcagccccctgcccctcctgcccgccgaagccgtactccgcgtggccctgttccccaggctactcctggctccacttctggtgcccctgctttgctggaactctcagccggccttcctatgtacactgtgccccgtcgcggtgttaacacctttgtccctgatgctcagatgctattccatgtccttggaatctgtgatcaaatgatgctttcaactgatcgctttacgcgttcatctcctgcttggatccccatcgtc

SEQ ID NO: 12 - DNA, 500 bp, unidentified
  misc_feature 1..500: note = unclassified Partitiviridae
  source 1..500: mol_type = other DNA
agaatttcctactgtgccacattattgtatacagttgaaatttaactctaatttttacacccgctctttgcactcttatattccacccctaccatggaaaacggagcaaataatcctccagctgccggcgccattgtcgcagcagtgcaacctgcactccccccgggtctcgacgcttcgaagcttaatcgtgctactgaatatctgtacgatgctgacaagatcagaccccatcgagctagatcaccaaagtaccgtcgctacatgcagctcaagaagaacgaaatctacgttcagctcatcgacatctatcgtgcatctttctctgtcacttggcctgattttcgccatttgattcatgctttcccgcccccggctaaccatgaaccccaaacgtactacgcttgctgctatatctccggatggtttcgtgatttatactaccatatccgtaaagctcttaagagcctttctggactggctgaactcgaatattatcg

SEQ ID NO: 13 - DNA, 500 bp, Primula malacoides virus 1
  source 1..500: mol_type = other DNA
gatttttttaaaccggccccccgcttaaaaatttagaatttacattcgtctacaaaatcaagttcttacaaaacccctgctctttttataacaatgtctgataccgtccttcaagctaagactaacttccttaagaataagtcacctgtccttagtgtccaagaaagcaaacgacctaaaatctcacgcgacctaatgccacttttccaaagtaacactgctgctcgctctgactcatccaaccaatggactattgatgtctttccaaacatggttcctattactatgtatatcatgctcacctcaattgctcacgccaatgatgttgaccaccgcactacctccaaagtgtccgttgcaactatttgcatgtaccatctctccatcgtctacggttatttccttctcaatgacatgattactcgtgcctccgcttctgctcatgcccgcccctggattgaaacttcctggaaacgcgaattcgccgactttttgctcac

SEQ ID NO: 14 - DNA, 500 bp, Fig cryptic virus
  source 1..500: mol_type = other DNA
ggattgcttccaccgttgcttatagcatcgatcacatcgttcaaaggctgacggtcatcgtggttagaggaaactcgcagcccagactagactcctacgaagtgctaacgttacggcatccgcctaacggtaggcagcgatccacaccactgctctcttgtcaagttttacatcactacaatggcagcacccaatttcgacattcttgaagacctcattgctgatgggcgcctccaaattaccccttatgtcacctcaactgaacccaaaatatttgacgtcgatttcgacgcactctggaacaggattaacgctattatagttccaatttacactaattgggatcaactcaggtttcattatcccggaacaactactactgtgacaaacaacatcacccttgacgttggacacctagtcacatttgcgctctggcaactttataacagactccgatctgttgctgaatgcttccaccacccaacgcgtacctcgcgaat

SEQ ID NO: 15 - DNA, 500 bp, unidentified
  misc_feature 1..500: note = unclassified Partitiviridae
  source 1..500: mol_type = other DNA
agaatttcctccaaacactagaaattcgtgttttgaggcaattaaaccattaattccaatggcagaccaaggtaataatgagactcctctaccagtccaggcaacccaaccagccgcaaacagcgatcgtataccgatcacgaccctccctgcccccaggtcacaagcccccaataaacctgaatacgcacggattgcagactccctggaagacgaatatcaaatcggattccaacggatcaatccacgacttcaccgtcgttacgttatcctcaggtcacaagagctccacgaccgattgtgcaacttgtatgcaccttattttcaagctggatatcgtgttttcaaacacgttatcgaagcttacccactaccagcgggatacgctagtcaaatagcatatgccactgcgtgttacgtgtcttcgtggtgctgggatctttacgtctcgatccgagaatcagttaagaagctatcacaaacagcgttcctccaacaat

SEQ ID NO: 16 - DNA, 500 bp, White clover cryptic virus 2
  source 1..500: mol_type = other DNA
agattattcttaatcggcccccgaaagaatttaagaattcacattcgtctatattatcaagtttttacacaacctctgctctaattccaagatgtctcctgatgagaaccccactctcaacaagacccctaagaaccaaactgttgaactcgtttttcaagataccaaagttcccgttccctctgataacttcatggaatcaatccgctctaacgctgctgctcacactgacactgctaatcagtggatcattgatgttttcccaaacatgactccaatacttctgtacgttatgcattatactcacttatatgctcaagctactgaatatcgcattcatgccaaatcatctgtttacactttgtgtatgtatttcatgactattgtctatggatacttcttgctgaatgatatctacacccgccctgcgcggtctgttcatgcccgtatttggattgaatcttcctggagatctgaatttgtcaaatttctgacgactc

SEQ ID NO: 17 - DNA, 500 bp, Red clover cryptic virus 2
  source 1..500: mol_type = other DNA
agatttttcttaagcgccccgcctcaagaaattaagaattcacattcgtctaaattatcaagttctcacacaacctctgctctaataattaagatgtctactgaagagacccttcctcctactaagactattaagaaccaaaatgttaacctcattttccaagacaccaaagttcctatcccttctgataactttatggaattgctccgctctaatgctgctgctcacactgatacctcaaatctatgggttattgatgtactccctaactttaccccaattctgatgtttattatgcattacgctcatatccatgctcaagctgccgattatcgcgttcatgctaagtcatctgcttacactttgtgtatgtactatatgactgtcgtctatggctacttcttgctcaatgatctacacactcgtcctacccggtctgttcatgcccgtgtttgggttgaatcatcctggagatctgattttgttaagttcctgactac

SEQ ID NO: 18 - DNA, 500 bp, Hop trefoil cryptic virus 2
  source 1..500: mol_type = other DNA
agatttttctaaagcggccccgctagaaattaagaattcacattcgtctacattatcaagttttcctgaacctctgctctaatccttaagatgtctactgaagaaactgctcctcagactaaggcaatcaagaaccaaaatgttgtcctctcctttgaagatggaaaaccacctggtcccgtctctgacttcatgtctttgttccgctcaaactcttgtgcacacactgatactgctaatcaatggatcattgatgttttccctaacatgactcccattctcatgtacgtcatgaccaaagcccaacaccattctgaagctactgactatcgtctccactccaagtcatctgtcttcaccatctgtatgtactacatgtccatcatttatggctacttcttgttgaatgatttacattcccgccccactcaatctgcccatgctcgcaactgggttgagaactcttggaaatcctcctttgctaagttccttatgacact

SEQ ID NO: 19 - DNA, 500 bp, Dill cryptic virus 2
  source 1..500: mol_type = other DNA
agattttattaaagcgccccgaactttagtaattaagaactcacattcgtctaaattatcaagctataacaaagcccctgctctaatataatacgatgtcttctacatccctcacatcccgtctcaatgccctcaagactaagacaatgaacctcacctttactgatgtcaaagcccctgtaccctctgctgatatccttgaactcttccgtgccaacactggtgctagagccgattcctgcaatgactggacaatcgatgttttcccaaacatgactccaatcatgatgtacactatgctgatgtccattcgacatgccaatgctactcaacagcgcgaacattccaaagcttccgttgctactatctgcatgcatcacatggccgttatctatggcttcttcctcgtcaatgaccttcatgtccgtcctgctccgtctgctcacgcccgctcttgggctgaaacctcctggaaagatgactttgtcaattttcttatg

SEQ ID NO: 20 - DNA, 500 bp, unidentified
  misc_feature 1..500: note = unclassified Partitiviridae
  source 1..500: mol_type = other DNA
gatcaaaagatcaacagccttccaacgctttttgcaatcaaaatagaacgcgaatctccgctgctctgactcaacttgttacctactctaccgttaccacaaccgcaaccccgaaatcatggaccccaacgtccctattgctcaacccaatggccccgctatcccaaacgttgccgctgccgctgctaaccctcctccaccgattcaaccggcccctcccgccccaggtgctgtccaacaagcccctgcccctgctccccgccgcaatcgcttaccccacggtcctgtgcccacatcttcccgcaccgccgtaacctccgctcctgctttgctcgagctcgctgccgcaactcctatgtacacggaacaacgccgtgctacaaacttctttgtccctgactctcagatgctcttccatgtgctgagtatttgtgaccaaatgatgaactctactgaacgatttcttcgttcttctcctgcctggatgcccattgtctcac

SEQ ID NO: 21 - DNA, 500 bp, unidentified
  misc_feature 1..500: note = unclassified Partitiviridae
  source 1..500: mol_type = other DNA
caactgctctacctcaactttttacttactctaccgctttaacaagccgctcgataaccatgaatcacaacactcctcctgctgacgccaatggccctgcccttcctgaaggctccgtccctccccctaaccctcctactcacatccctgtagcccctgctgctcctggcgccgtccaaacgccgcctgctccccctgcccgtcgcaatcgccaaccccatggccctgtgcccgctgccagcaaccaccatgctcctggagctcctgctctcctcgaactctccgctgcctacccgatgtatactgaacaacgtcgtggtgttaacttctttgtccctgactcccaaatgctctttcacgttttgggtatgtgtgaccaaatgatgaactctactgaccgatttcttaggtcctcccctgcttggctccctatcgtctcccagctttacatagctgttctctggaatgttatgatctgccgtgtttacataaacactggc

SEQ ID NO: 22 - DNA, 500 bp, unidentified
  misc_feature 1..500: note = unclassified Partitiviridae
  source 1..500: mol_type = other DNA
agaattttatcctgactactattttcagccaggttaagatactaatcttttcatacactacgctcaaagtgcaattaccgtcatcttacaatgacaactggtgatcgtgtaacacctactgacggcattacaaccgtcgatgcacccacagcaactctcgaggctaagcttgactcttcgtctaccgcttacctcaaaagcgcgaccgactttctcactcagagtagtcggattggtaccttgaggaccagctacaaggaccgccgtagatacgtctacttaaagtcccaagctatgtttgactcccttgtcgagttatactctcttctctttaccacccagtggcaagttttccgcaggtttactgatattttcgttcctcctgccaacgttactgccacccgctacctagccatgaactacatctctatgtggattcaggatacgtatgcgatgaaccgcgctgccctcatgaaattatcggtggtagctttaaatga

SEQ ID NO: 23 - DNA, 500 bp, unidentified
  misc_feature 1..500: note = unclassified Partitiviridae
  source 1..500: mol_type = other DNA
gaaattactaccaatttcaaattcccacgttataattaacccaacagatattctgttcccttatatagctccccactcaccacaacaacttgtctacttacaatgtcgatgaaaagaaagtcaaggcctagttcttcaacaaaagatttagaggtagaagacacctatctcaaacaatctggacttgattcaatgaataagctcgaaccagtcgagcaatcaaaagacgaagagactaccaaagtctccatgctccccaccgcttctactgctataatcgccccgcgtaaactaactgcggaagattttagctctaaacgtaagccggatcaaacgtccgctgttagcccattctttgggttccttaggacccacatcctacaccctacacagggcaggctctcacactactacccttcctgccacatgatggactacattcttcactctatcaattcaactctctgtgataattactacttcaagagagaaactccaaa

SEQ ID NO: 24 - DNA, 500 bp, Cannabis cryptic virus
  source 1..500: mol_type = other DNA
agatttttctaaagcgccccgcctttagttattgagaatttacattcgtctatattatcaagtcctcgaaaaacctctgctctaattttaagatgtcttcctcaaccatcacttcccgtctcgatgccctcaaaagcaagaacgtgataattgaattcaaagaagcgaaaactcctgtcccctctgctgacattttgcaactctttgctgacaacaccgccgcccgctctgactcatccaatgaatggattattgatgttttcccaaacttcactcccatcctgatgtacattatgctcaatgcgattcgccacgcgaacgccactgctcaacgtgaacattccaagtcatctgttgccaccatttgcatgtatcatctgtccattgtctatggatttttcctcatcaatgacctcaatgtccgtcctgctccgtctgctcatgcccgtatctgggccgaatccaactggagatctgaatttgtgaaatttctgtccacc

SEQ ID NO: 25 - DNA, 500 bp, unidentified
  misc_feature 1..500: note = unclassified Partitiviridae
  source 1..500: mol_type = other DNA
gattaacagccttcataacgctttttgcaatcaaaatagaacgcaaatctcagctgctctaactcaactttttacttactctactgcttacccaagccgcccacaccacaatgaatcctgaaacaccacttgccgaccccaacggtcccaccccacctcacggtgctgctgcccaacccacccctggccaacacatccctgctggaactccctccactggtgctgtccacttgacaccagccccgcaacccaagcgcaaccgcgcgccccgtggcccgttcgacaatgcctcgagcacctctgctcctggagctcctgctttactggagcttgctgctgcctaccccatgtccacggaacaaaggcgttccccaaacaccttcgtccctgacgcccagatgcttttccatgtcctcagcttatgcgacaacatgatgctgtctaccgaccgtttcacccgctctgctcccgcctggctccccatcgtctcgcagctttac

SEQ ID NO: 26 - DNA, 500 bp, Pepper cryptic virus 2
  source 1..500: mol_type = other DNA
agaattttctgatcacagtatcgagagatcactgtgaccaaagaaagttattaaaatttaacctaagacgtttataatatggcgacaccagttagtgacacaccaactactaagtctacacaagacaagactgaccaattaccggcagctacgccccctataacgcaaaagccaccctcctacaaatccgagatttcaagaatcgccgactacctcgccgatgactacgaaattggatacgtaagagaccgtccacccaagcatagaagatatgctgtgttaaggacgcaagctttattcaacaaacttatcgaaatctacggtaagttatttaaacagaactgggaattcctccgacgttcaatcgacggtttcacaaccgcagttggcattcgtgagagacaatgggaaaacgcagcccacgcgtatatctcatgctggttctttgacctttacgtttctgtcagaaacgctgttgaaaaggtctcaggtactgcatt

SEQ ID NO: 27 - DNA, 499 bp, unidentified
  misc_feature 1..499: note = unclassified Partitiviridae
  source 1..499: mol_type = other DNA
agaatttctgtcttggcctccgaccaaaaccgaagagaccaagaaggaagattattcacccttattagcaatatatctcaccactctcccactatggcagaaaacgatattccagccgaaggaatcccagcaagacctccgcctgccgctaaccccttcacgggattaaaccgtgatgaactcgaccgcatcgtcgagcaaccagaagaggataaccaaattggtttattccgagataaggctagatcttatcgaagatacgtagacttacgtacctctccgcttagtacaaggttaatcaacttgtactcaaaccttttcaccactcactggaacgtgttcaagcacaccatgtctggagaagcccccgccggattcgcctcaaaagcgatctacctggcaacgatctacgtatccgcatggatatgggatcttcatgtctccatcagagaatctgtgagaaggatctctggtactgctttcaaccagctgtttcagc

SEQ ID NO: 28 - DNA, 500 bp, Pepper cryptic virus 1
  source 1..500: mol_type = other DNA
agaatttgacctgaaccccatatcaggatcgaattatttacaaattcaccaaacacgtctaagcttgtgaaaattattgttatccctacattaacatgggcgatcgtgttaacgctcaagacgacgatacagtcgtccctcaccaagcacctcttcaaccagcagctcttcagcaagacctcaccagatctgccgactacttattagacaacgtccgtattggcaaccaccgacaacgctatgataaataccgtcgctatgtccttctcagatccagcgaaatattcactagtctcgtcgccatttacgctcacatcttttctagttattggcaacactttcggaggttcactgatcaatttcaagctccaactggagtccaactccccacttttgtggcacgagtctacatctccacttggcttcatgatctatactgctctattcgagaggccactagaagtatttctccattagcctttaatgagaggtactcatat

SEQ ID NO: 29 - DNA, 500 bp, Carrot cryptic virus
  source 1..500: mol_type = other DNA
gatcaaaagatcaacagctatctaacgctttttgcaatcaaaatagaacgcgaatctctgctgctctgactcaacttgttacctactctaccgttctcaacaaccgcactctctcactatggatgacaatactcctgctgcccaacccaatggcccccacgctcctgctgccgctgccccccacgctgttccccctcctgcaatccctcctgctactcctgcccctggtgctgtccaacaaccacctgctccggctccccgtcgcagtcgcaaccctcatggccccactcccatctctagccgcgtatctgctccttctgcccctgctctcctagaactccctgctgccacccccatgtacaccgaacaacgccgctccgccaacttcttcgtccctgacgcccaaatgcttttccatgtgttaagcatatgtgatcagatgatgaactctactgagcgctttctccgctcttctcctgcctggatgccaattgtctctc

SEQ ID NO: 30 - DNA, 500 bp, Crimson clover cryptic virus 2
  source 1..500: mol_type = other DNA
agatttatttaaagccgccccaagctctaaatattaagaattcacattcgtctaagttatcaagttctcacaaaacctctgctctaattatcaagatgactaccaactcatcaaacgcccgactcaataagctgaagaataacactgttcctcttcctttcaatgctactgaagtccctgaacccgctactgacctcctcacttctatgaaggagaatacttctgctcactctgctactgctaatcaatggaccatcgatctctttcccaacatgattcctgtcctaatccacgttatgtctgctgcctcacaccatgctaacgcgattaactttcgtgaacactcgaagacctctgctgctactgttgccatgtaccatatggccgtcgtttatggatacttccttttgaatgatctgaacattcgtctcaatccttccgctcacgcccgatcctgggctgaagcctcatggaaatctgaatttgttgaattcctctac

SEQ ID NO: 31 - DNA, 500 bp, Beet cryptic virus 2
  source 1..500: mol_type = other DNA
agaattatcatcttagtcttgcgataattcagttaagactaagaagtaatcttaatttacctattaacgctttattgtttcgtacaatactacccatagcgcgacaatcataatgactggacccacacaggcccacctcatcgaagcgacaagtttcacgggactcaaccgtgacgagctcgacagcatcgtggacgccttacaagaggagaaccaaattggtacctttcgtgacaggccaaaagcctacaggcgatatgttcacgtacgctctcaggagctctttgatcggctaagcgaactctatgccgatttatttacaagtaattggtctgctttcaagcatgcctatattacagagccaactggccgtgaagatgcaaacagcatctcaacgaaagcctctttcatggctcaatgttatttgaccgcgtggttctgggacacgcaatactgcatacgcgaaagcgtaagaaagttgtcaggcactgcctacaacc

SEQ ID NO: 32 - DNA, 500 bp, Beet cryptic virus 2
  source 1..500: mol_type = other DNA
agaatttcaccttagtctcagaaattctttacagagactaagacgtgttaaattattttacccatcacagctatataacttccaacttcaaccctacaccgaagaaagactttactttcccattcaatggcaccgaatgacgaacttgctgcaaaccttcaaggactcaaacgtcctgctcttgacgaaatagtgcattatcttgaggaccgtaatcaaatcggtatatctcacgataagaacttgtcctacaataggacgtgtgtactactcaccaatgagttacatgccaggctcgtgactctttacacgaacctcttcaccacttcatggcgacactttaaaagtgccgttcgtaaggaaatgccccgcactgaagccaatccacaacgctggctggccaactgctacataactgcatggttttgggatctccaagcctccatccaagaagccaccaagaccttgtctggtaagatttaccaagactacttccgaga

SEQ ID NO: 33 - DNA, 500 bp, unidentified
  misc_feature 1..500: note = unclassified Partitiviridae
  source 1..500: mol_type = other DNA
aattgaattgcattctatctcggtcttataaacttaaagaattttagtcacagcagattttattttctactcccctcccccacattatcatggacaaatctaagaagtatctatctgatgctaaagaaatcatcaaggctaatggtcttgaagaaaaatttctcgccgctgctggtctttcatctatggatgaactcgaccctaagaacttcgctgagtttgaagctcctgctcctcctcaagttatgaagccctctatcgccgctgcccccatcattcaaaaaccgctttccaagcaggttgctgatgcctctactggctttaatccagagagcgcttctgatatgctcagaccatttattggtctaaagattaactacaaagctaggaagactgtatctacctatgctccgtcctctcttatgatggattatattctccatcaagttaacaatttacttgttgataatttctactttcgtcgggcttgccccgattac

SEQ ID NO: 34 - DNA, 500 bp, unidentified
  misc_feature 1..500: note = unclassified Partitiviridae
  source 1..500: mol_type = other DNA
gagaattattcaatataaaataatcaacacttagcattattttccttactagcgcccactatggcagctttaggtgatggtgttccaccgattgcggacaataacaacccgcaaggaagagatgcaaatcccattaggggaggaactgatcccctacgaagagcaactgactggttggaagacaatagtaaaattactatgacgttccaccgtcatgctaactaccgaaggtatgcaacattgcgccttcaagagatgtttaatgctctggtacaattgtatcagaacatattcatgtcccattggcatcatttacgccggacagttgagaactaccaagttccagctccactggctcagacgccatggctttatgtagcacgtctatacatttctgtatggattgttgatctttacgcctctatacggcgcgcatgcttacgcgtgtgccctgaggcatacaacgagtattatactcaagattatcagcaggtagtctt

SEQ ID NO: 35 - DNA, 253 bp, synthetic construct
  misc_feature 1..253: note = synthetic construct
  source 1..253: mol_type = other DNA
tgctaattctctttttatattaatttaatttttaaattttttcatgtttaatatgtttttctttaataacaattaattttattatatttgatttttttttttttgattttttttttgtatttgaactttagaatttattattttttaatgttattttttattcttgaaaaaaaaaaaaaaaaatcaaaaaaaaaaaaaaaaaaaaaaaaaaaaaattacataaaaaacacaaagaactctaaaacaccttccc

SEQ ID NO: 36 - DNA, 500 bp, White clover cryptic virus 1
  source 1..500: mol_type = other DNA
gataccaacgtccgtgactggctctaccccgctgctgctgctattgctcctttccgttctacccgctttttaccgcgtcgtgaactacctgcgaccctagctgtccgtttcgcccatgccgatcatgagatcgaagaacaagctgaacagtattccatactgtgtcacaccaatatgaagtggtatgtcaacaacgccactcagaacaaccacacagctattgaaggaaactacattcaccaaggtgagtactggaacttcacccctttccgctactctcctcctgtctctctgaagacccagtttgctcaagtgatcgcttcccgctaccaccaacaagctgccaaccgtgctgaatgattcctccgacctttgatgtcgttaatctcaccaattttgctgcctaagtgcagttaatctgatcagattggattttttttgttttaactttcttttaatcaactttatccttttaattctgacctattatcgctcttgcg

SEQ ID NO: 37 - DNA, 500 bp, Vicia cryptic virus
  source 1..500: mol_type = other DNA
actggtcgattcaaccctcgtcgtgaaatcccggcccaactccgcatccgcttttctcactccgatcatgaactcgaactccaagctgaacagtacgctattgctgcgcacaccaacattaggtgggctgctaacatagctacacaacatgaaaggaccgccatcaatccggctcatcttcaccaaggtgattactggaacatgacccctttccgtcacaccggacatctcggcctcaagactcagtatgcccagctcatcgcttctcgctatcaccaacttgccgccaaccgtgtcgactgacaacctcctgacctagaacgtcaataaacttctaattttgctatctttgatagttattctgatcagaatagttttttgtttttttccaactcccaacttagttttctttaatacaaatttttatcctctctgacattttgttcgcccaaagtaaaaaaaaaaaaaaaaaaaaaaaaaaaaaaaaggaaaaaaaagtt

SEQ ID NO: 38 - DNA, 499 bp, unidentified
  misc_feature 1..499: note = unclassified Partitiviridae
  source 1..499: mol_type = other DNA
tgacgaggatgccccacctgtcattgccgaacctggctactactaccccaatcccaaccccgtcctctctttgacgctcgtacctgcattgaagacatctcttctgcacacatgttctccgctatgaccttccatcccaatctcataccatacggagctaacagagctaactttctgaaaggacagttttggaattgccaccccccaagctacagcgctcctgcccaccaggtctatccagctgtatccgcccttatcgctcgtgagtatcacagtgaaactaacctttcatctgacaagtgactctctcgacacccccgacacccctctcgctgatgacgaagatgtgaccggagtagacgctagcgcgttccttcggaatttttcggttcttttgttttgtcgttagctacgactttttcttatttcaatgtaaaccctacctattagctatcttttaaattaaaagaaaaaaaaattaaaaaaaaaaaataaaacc

SEQ ID NO: 39 - DNA, 500 bp, unidentified
  misc_feature 1..500: note = unclassified Partitiviridae
  source 1..500: mol_type = other DNA
ccacggtacatcagatctcgtctgcctgcttcctcctacaaactactctccgcttgctgttgcaacccgaatgctcttcctcgctactcaggacgattctgacgaatgtactgattttatcgaatggccagaagatctcggtgattatggaacacagctgaaagaagccccacccaactccaatgttcgtgcatttcttgccttatgccatggacccctagaagaatggagcaatggacatgcttaaatatttatttatttatggtcatgtaataataacacaacccctaagttttgctgaactcttaagttctacttgattttacaatcgctaagaactaccttatatttatttatttgtgtatttctcttaaacgctgatacttttacgtatcacatcccgcacacacacacgggatggtgatgtgttatcttctagacggacttatcgtcaatgacgataatctcctactagaaggtacacagcaccctctcatatc

SEQ ID NO: 40 - DNA, 500 bp, unidentified
  misc_feature 1..500: note = unclassified Partitiviridae
  source 1..500: mol_type = other DNA
ttacgccagcactttgaagacggaatcttcgagctgcaactcccacttccagaagtgaactttacttcttcgatggctttgaccctatctctgtttctaaatgcagaagaagtcaatgctactagcgagatattcgacctcacccctgttggagctgatatatacggctacatcatgcgcgaaccacacctcggtatcaatgtttcaacatttgaagttattgatgaaagcgccaaggagatcgtatctaatgtttaattatttattaattaacgcccagacaatgtggaccatgtttaattttaattctgagacaatttgaattattcggttttgtaaccttaaactatgttggtttaattttacatctgtgttaaaagttaactcgtttatgcctagattctctgaatcttgtaaggttgtactaaaatacatctttatgcaaaactttcgagtgatgcggcgacctaccttctataggccgcccgcagcaccttatc

SEQ ID NO: 41 - DNA, 500 bp, unidentified
  misc_feature 1..500: note = unclassified Partitiviridae
  source 1..500: mol_type = other DNA
cgctgcccacttccagaggacaactacactctcgcaggtgccaccgtttttatgctcttctaccatgacgttggatcggaccccgcaatcgatctcttcaacatcgactctcttggcaatgatgactacggctcattcatccgcaacccacgcgatggattcaacgcctcgtcctactacgccatctcttcagagggcaccgatgaaatgtggaaatcgtccgcttagtacaagcgccaaatcttgtctaaacgggaatacccgataatttgtgtcgttctatttaaactttatcgatttaaatggtctgtaataatgaactcgttaacttgagctttattttaagtgtgattttctttcctacttggttttgcaattattttcttgctcctctcatctaatcaatagatgttgaggctcttctaagtaggtgcagtttagaaaagtttagtcttatgactctacttctccattctaaacgtgcacactacactatagtc

SEQ ID NO: 42 - DNA, 500 bp, unidentified
  misc_feature 1..500: note = unclassified Partitiviridae
  source 1..500: mol_type = other DNA
gatcaactttccgccgcttcacttcaacgaccatctcgcaaatcttgcgatcatgtttctcggtaactccggtgctgacaggaacaacgcttctatcatcacaaccaagacagatgaccaagactacggctggtacgtcaaagacatccgcgaaggcgaacaagctcgcgcttttgctgctctctgccagtgggacccccgctactggaacgagaaaaataatgtctgaagtcccttgataatttgtgtttgaactactttgatgttttcaaactttacttttattgtcattgtatgaacttattaattatggaaatctctttctctaatttcttatctctagttaatcaaattaactgactatgcaaaaatataactacttaatgaagcataatgcttcaacaaccaagtggtgtgtctttgcagaactcgagaacgatctgaggatcgtttctccatccgatggatgtgattctgcaagtcacactacacaaaatttc

SEQ ID NO: 43 - DNA, 500 bp, unidentified
  misc_feature 1..500: note = unclassified Partitiviridae
  source 1..500: mol_type = other DNA
agcactacgaggatcacgtcttcgagctgcaagtcccgctacctgaggtaaactttacttctgcgatggctataactcacactttattccttgaaggagaagctgctgacccatcaaacgcggtattcgatctcaccccagtcggagccgacacatacggctacattctgagagagccacacttcggaatcaatgttaccacataccaggtcattgacggaagcgctcaagagatagtttcgaacgtgtaacttatgttacgaaaccgataatgtgaacaatttatgtttgaatctttcctgagactattcattctggttgttttgtaatatttgaactatgttggtttaattttacatctgtgttaaaagtctactcgttacaactgcctagggctgaccccctgccgtgatcggaagccccccctttcatttcacgtgctttacttacgagtgatgcggcgaccccaaattttttgaggccgctcgcagcacctgatc

SEQ ID NO: 44 - DNA, 500 bp, unidentified
  misc_feature 1..500: note = unclassified Partitiviridae
  source 1..500: mol_type = other DNA
acttacaagctcaacaatgcccatggcactaccgatcttgtctgtactatcccaccaaccaactactccgatcttggtgttgcgctgcgctcactatttctggctactgctgctgactctgacgaatgttcagaaatcatcgcctggcccgagggcactgccgactttggcaccaatctccgcgagaacccacccaactccaacatccgcgcatttcttgccttatgccacggacctctagaagaatggagcaatggacatgcataattactaactatttatggtcatgtaataataacacactacttaacatttgctgcttgttttgctagtatcactaaaactttgatttatcaggccaactcgtatttatttacacttttatcgacggtacttttacgtatcctcaacccacacacacactgggttggtgatgtgttatcttctagcttagaaatcgtcattagacgattcatgttagaaggtacacagcacctact

SEQ ID NO: 45 - DNA, 500 bp, Beet cryptic virus 1
  source 1..500: mol_type = other DNA
ttcctttgcagccgcttcgccccaagaagagaaatcccgaatactctcggtatgatcttctcccatgctgaccatgaactcgaggaacaagcagaacaatatgctatcctcacccataccaacatccgttggtctccttccgtggtcgctcaaaatgcgtggaccgctgtcaatgacggcgcgtctcgcaatggtgactattggatcatgatgaactaccgtttttcaacgcgtatttcactcaagacccagtttgctcaagtgatagcctcacgttatcatcaacaagcagcaaaccgcgttgactagactgtcacgcctttgctaatttcgtcgccctcctcactgctttttgcagcctttctgatcagatagggtttttttttgtttaatctcatttctatttaaatttcacctccttcagaatctccttttataggggaattcgtttcgttttgttttgacgttcgaagaaaaaaaaaaaagaaaaaaaaaaaaaa

SEQ ID NO: 46 - DNA, 500 bp, unidentified
  misc_feature 1..500: note = unclassified Partitiviridae
  source 1..500: mol_type = other DNA
cctccacgtccaggaaaaggtctcgtaccaccagctcctctaccccaggctcactggtcatctgtaagtatatctgtttgtatccataccgattacattacctccctgttcatattaacatctcactatttaattagctgaaccgacccaaagtcacgctcaaacagctcgcgctgaagaagaagactacatctgcgcaccacgcttccgtctcatcgactggtgctacccgttccgcgtcatcatgggatcgtctgctcaagacaaatatgcggctcaccgcatgattgcttttaattaattttaattatgctttaaaacaatctccccttttggctatgtattaagactttatggatttaacctttaatggtttatttattttgctctttgcattttatctagtattatgtgatttgaatcgcataatggcagcaggatttattcgatcgggtatataccttaagtatataccaacaagatcatttaaagaccttgcc

SEQ ID NO: 47 - DNA, 500 bp, Primula malacoides virus 1
  source 1..500: mol_type = other DNA
ctttttacgttatgcgcaatctttcttcggttttcacaccgtagattctagttcaaatgctctttctcttgacaccattcccggaatgacgaatggtctgttacacctctggtctccatactcttacactccgtttgagaatgactactttccagacccagacctcgctgaatcaaggcactacttcttgtccaatctgcgtaccatttttggtactgactacaatcttgttcaagccaagcacccttatgaagctctccctgttgtttaggctgattctctctttttctcgaaattttatttctagttttgatgtttattaggcttccctttcaacaactacgtgtatgctacgtagtcttttgtttcgattttatttgtacctgtactttcttttttcgttattttctgttctagaaaaaaaaaaaaaaaaaaaaaaaaaaaaaaaaaaaaaaaaaaaaaaaaaaaaaaaaaaacaaaaaaaccaaaaaaaagttccc

SEQ ID NO: 48 - DNA, 500 bp, Fig cryptic virus
  source 1..500: mol_type = other DNA
gttggattcgtcaccgacagaacagtcatggacccacttgcggcactagctgcaccaactggcactccaagatctgaagaagcaacatcggcaggcagacctgttcatggcatgtacaatgtcaaccaccgcggcatacaaccagctgcaggtgaacagaacccctcaggctgctacatcattggtcgtggctatgaacgccgctacacatgctacctcgcacgccgaatcgctgttttcgaggcgaaccgcattatccgttatcgctttatgcgttaatttgctatttaattgcagtttaatttctaattcagtcgtttatcagacaatttaaactatggtttgtaatgaacttatttaaatgaatttcagtttttatggaattgtattatgattttataattgttaaaacttaccatgtattggtataatacgcttgactgatgaatttctctttggtgtatatcatgtatgcaccggagaatccatcacgcctaccc

SEQ ID NO: 49 - DNA, 500 bp, unidentified
  misc_feature 1..500: note = unclassified Partitiviridae
  source 1..500: mol_type = other DNA
ttcttcggagcagcagagtatcggacgatttccactcgcatgtgggaacttcccttcctatcagctgtcgccggacagatgtcaggagcaaagcgcccagctccagaaagctcggccaccggaagcggatacatggaatcacctccagccgttgaaggtcaacagacagcagcaattatggatcctggaataatgcaaacgcgttattgtcagtatcaaatcatcgattggtgttatcatggaagagtaatcctcaacctgaacgctcagaagcaaaatcaagctctccgctcaattttgttctctccaaattgatgtactgttgtaatatttcgttttgttgtaattcgaactgcttatgtataacttacaattttgaacccatgtaagtttaacatttgattcaatgaaattcgcttttcgcaaaacggggtaacaggctactgtaacgactataagtcccctaacaaggtcttatggcggaactacagccccttact

SEQ ID NO: 50 - DNA, 500 bp, White clover cryptic virus 2
  source 1..500: mol_type = other DNA
tcgctatgtccagcccttcttcggttttcacactgttgatcgtactgctaatgatgctgctcttgatgctgttcccggtatggctgaaggccatttgatgatctggtccccatacacttacaatccgtatgagtctgatgattatcctcccgtcgcccttgatgcgtctagacactactacctgactaatctgcgtaccatttttggtactgactacaatcttgttagtgtcaaacacatctatgaatcctaccccgctgtctaagtgattactctccttttcttttaattaaagtttaaaagtttttgatgtttaatcgttttctttccagcgaacacgcctacactgcgtggttcttttgtttcgttttaattttatgaacttttgtttatgtttttttcaacgttgttttcttattaagaaaaaaaaaataaaaaaaaaaaaaaaaaaaaaaaaaaaaaaaaaaaaaaaaaaaaacaaacaaaaaataaaaaacccc

SEQ ID NO: 51 - DNA, 500 bp, Red clover cryptic virus 2
  misc_feature 348: note = n is a, c, g, or t
  source 1..500: mol_type = other DNA
ctatatccagtcttttcttggatttcgcactgttgatagttctgccaatgaagctgcccttgatgctgtccctggaatgaatgaaagcttgctgatgatctggtccccatatacctacaacccatatgagtctgatgattatcctgctcttgcttatgatgcctctaggcattattacttgaccaacctccgtaccatctttggtactgattacaatttggttgccaccaagcacatttatgagtccttccccgctgtttaagcaattactctcttttccttttaattaagttaaaagtctttgatgtttaattggttccctttccagcaatcacgtctatgctacgnggttcttttgtttcgttttttattcctgaacttttatgtttaatgtttttcaacgttgttttctatttatggaaaaaaaaaaaaaaaaaaaaaaaaaaaaaaaaaaaaaaaaaaaaaaataaaaaaaaacaaaaaaccaaaaaaaagttccc

SEQ ID NO: 52 - DNA, 500 bp, Hop trefoil cryptic virus 2
  source 1..500: mol_type = other DNA
gctacacccaatctttccttggattccgcacagttgatagttccaataatgctgcaaacctcgatgctgtccccgggatgactgaaggtcttctgatgctctggtctccctacacatacactccgtatgagtctggtgattatccattcgctgatctcagtgcttctaggcactattatctgaccaacctccgtaccatctttggtactgactacaacttggtttcagttaagcatccctatgaagctttccctgtcgtttaagcaattactctctttcctttaatgcaatttaaagtttttgatgtttaattggtttcctttaatgacaatcgcgtgtatactacgcggttcttttgtttcgtttttcttctgaactttttagttcagcgttttcaacgttgttttctgtttatgaaaaaaaaaaaaaaaaaataaaaaaaaaaaaaaaaataaaaaaaaaaaaaaaaaaaaaaaaaacaaaaaataaaaaagttccct

SEQ ID NO: 53 - DNA, 500 bp, Dill cryptic virus 2
  source 1..500: mol_type = other DNA
gttacattcagtcctttttaggatttagaactgttgatgcctcctccaatgctgctactcttgatgctgtcccgtcgatgcccactgatcgcctaatggtttggtctccctacacatacaccccatatgaacctgatgactatcccattcctgatcttggcgcttcaaggcactactatttgaccaatcttcgtaccattttcggtactgactacaaccttgttcaaaccaagcacccttatgaagctctccccgtcgtgtaagcaattactctcatcccccttatttaagtataagtttatgatgtttaattggttcccttctcgacaaacacgtgtatgctacgtgatttgttttgtttcgtttatttcgtgaactttaatatattcacggtttttaaccgttattttctaattaggaaaaaaaaaaaaaaaaaaaaaaaaaaaaaaaaaaaaaaaaaaaaaaaaataaaaaacaaaaacaaaaaataaaagttcccc

SEQ ID NO: 54 - DNA, 500 bp, unidentified
  misc_feature 1..500: note = unclassified Partitiviridae
  source 1..500: mol_type = other DNA
aatttgctcattcagaccatgaattagaagagcaagctgaacaatatgccatgctcacccacaccaacatcaaatggcatgcgaacctcaccgcccagaacaactggactgccatcactgctgacgggctctatcttggtgattattggaacatgatgccttacaggttttttgctccacttcactacaaggcccaatttgcccaaatcatcgcctctcgctaccaccaacaggccgcgaaccgcgccgaataggcttcgaccaacaacaaaattcatcgtgcaaaaattttttccgctcccgcgggctatttgatcagaaaggattttttttgttttatcaatttaacaattctttaccttttttttttttgcaagcacttcaagggccgtttttttttaaggttaatttttttgaaaatgggaaaaaaaaaaaaaaaaaaaggaaaaaaaaaaaaaaaaaaaaaaaaaaaaaaaaacaaaaaaaataaaaaaactgcc

SEQ ID NO: 55 - DNA, 500 bp, unidentified
  misc_feature 1..500: note = unclassified Partitiviridae
  source 1..500: mol_type = other DNA
acctttcgccacatcccgctatgctccccgccgtgaaataccaaatcaacttgctgtcaaatttcaacatgctgaccacgaggtcgaagaacaagccgaacaatatgcgattctcacccacactaacattaagtggtacgaatcgcttatcacccagaatgcgttaactgccgtcaccggtaacctcgtccaccaaggcgactactggcgtatgatgccgttccgctattctagtcccatcagccttaaggtgcaatatgctcaaattattgcctctcgctaccaccagcttgctgccaaccgtgctgaatagacctctgaccctgatgtcaacaaactcactttttgctgtccttaaccgatagtcattctgatcagattggatttttttgttttgaatttaactttctttaactttatcttttccttctctgtcttttctttccgcccttgcgggaaaaaaaaaacaaaaaaaaaaaaaagaaaaaaaaaaaaaaaaa

SEQ ID NO: 56 - DNA, 500 bp, unidentified
  misc_feature 1..500: note = unclassified Partitiviridae
  source 1..500: mol_type = other DNA
acatgcccggccagaccggaacaacaactgacagtcgtaagcgcccacgtcccggcgagtcaacctcagcccctgctctcccgcagacatcggagccgcaaacgcaaatgcaagtcgacactcccccagctcccaagccaagattccgtctcgtagactggacctaccactacttggtcgttctcaacaaggaggatcacactcgcttcgctgccctcaaaagcatctgctggaactgaacccagcaatttagtaatatttatctattattatgcttttctctgaaccctctctctgcaactttaaactctatttcttatggttttctgtttagactttatttatttaagtatttcgtactttaactaactattcgtactaactatttgtttaactaactttctagtttgagcattggtttgcaaaatggttcaggcccacattaaagtgtgggcttaatttactgattaagccttcccactttacgtaggacccgtgct

SEQ ID NO: 57 - DNA, 500 bp, unidentified
  misc_feature 1..500: note = unclassified Partitiviridae
  source 1..500: mol_type = other DNA
gagacttttgctgatccccgcttctccgccgatccaagagccctctatcccttcagtttcaagctgaagagcaccgcccacaacctccccccacttgcggaagctgccgcagccttctcccagacgcacatccggatttttccggaatacccgttggccggaaactttggtcaaaagaccgacgaatcaggccccttttgggacatcaggcccattggctccagccccaccgacgacacctcctacctcaccatcccacccatggtcaaggcagcactcatcgagaaaggctccagccgttaggagtcatgcactgacagacatcagccgctgaccaccttttttctttttcctagtatctcggatttagaaagcagcaattttacaaaagcagtcccattcggactagctgctttttcttacgcatttgcgtttgcttttcccttttagtattccttcttttattactaaatgtataacttaaattaaaaagaaaaaaa

SEQ ID NO: 58 - DNA, 500 bp, Cannabis cryptic virus
  source 1..500: mol_type = other DNA
cgattcaacctcgctatcaatgaagaacgcattggcaacattctcccaggaatgactctacttgatggagtcactgtcctccggtatgttcagtcctttctaggattcaacaccgtcagccgtgctgacaatgccgataatggagacaatgtcccggcgatgccccggaacctgcttctggtctggtccccgtacacgtacactccgtatgaatctgatgacctgcctgaaccagcacttggcgaatcaaggcactactacttgacaaacctccgtaccatcttcggaacggactacaaccttgttcaagccaagcatccctatgaatcgctccccgtcgtttaagcaatctctctcatcctcttaatgtttaagttttatgatgtttgattggcctccttttccgacaaacacgtgtatgctacgtgttttgttttttttcgttttatttcctgaactttttattttcagtcttttttaacgttattttctactttaag

SEQ ID NO: 59 - DNA, 500 bp, unidentified
  misc_feature 1..500: note = unclassified Partitiviridae
  source 1..500: mol_type = other DNA
ctctcctcattcaagtctagccgcttctccgccctcagagaaatccctgctctcctcaccgttaggttcgagcatgctgatcacgagcttgaagaacaagccgaacagtatgccattgccacacatactaacatgtgctggtctaagaaccttacaacccagaacaactggactcaagtagctgaagccaatgtgcactcaggcacttactggtctatgacccctcatcgggcacacgcctctgtaaggctgaagacacaatacgcccagctcgtggcctcccgttatcaccaacaggctgccaaccgtgtctaagtttggacccagaagtccttaaactctaacccattttgctgtcaaacagtctatctgattagaaagggttttttttgctttaacttaacttttatctttaattttctctttcctatagtgattcctcgcttcctcccgaagcgtattcttggaagctcagcttccgacctaggaacaaaaaaaaa

SEQ ID NO: 60 - DNA, 500 bp, Pepper cryptic virus 2
  source 1..500: mol_type = other DNA
cggaagcgctgaagttcgaacctatgctgaagagaaagtaaaccttactcgtttcacatctacaggtcacgaaacttcgaagaaaagaagtaagccgtcctcaaccaccacgacctcaagtcaaggcggatccagggacatggaaacagaagatgaacctcgtgaagtcgtaactgatatcacattatcacaggttacatccactcaacaactacaaactctaatgactacaggagaagcagataggtttaggattatcgactacacttattattgctgtgttattcttaggaataccgctcagaaaagagacggagcaattagaagtttctgttttaagcagtgaatttaaattatgtattgttatctttgctattaatgaaattatgttttgttaatatcgagcaacgttaactcttttaattttcgcttcacgatcttaaaattgggcataaggcactaaccttgtattaatttacaagaggttagtaccccttgtc

SEQ ID NO: 61 - DNA, 500 bp, unidentified
  misc_feature 1..500: note = unclassified Partitiviridae
  source 1..500: mol_type = other DNA
accatcccacctcctgacgcagcgacttcagcagcctcgcaaccaggaaagagaaaacaaggcgattcatccacatcagcaatggcgctaatggaaacagacaaggacccaccgcagcccttcgttccaccaccgcctgtggacctgaacagataccgtctcatagactggtgctaccacgcacgcgtcatcgacggcgaagatcagcagactcgcaaccgcgcactaaggaacttcatattccaagcaggccaccaacctacttagtagttcaagcgttaacataatttaattaatatgtaacgtttaaactttgaaccttctttattgttcttgaactctcttcatttatgcatttgaacgtttatttattgtttaatgttttgatttaattcgatcttaaaccatgaatgcaactttcaattgccttaaaatgggacgcagacataacgtacatacgttagagatagcactctaacgcagtcgttatgacccgcgct

SEQ ID NO: 62 - DNA, 500 bp, Pepper cryptic virus 1
  source 1..500: mol_type = other DNA
ccgcactcgtgaaactgaaaataactatttcttaccagatagtcttctcaacatcatcgaggattttaccgcaacaggtacaacacaacgaaggaagatccgtcgcccttcagctacgtccgcgtctaccggagccgctatcattatcagagatacgcccggaacagcgtccactgcaaccacgtccactacggagaccgaagttaccttccctccagtcatcaggactaagattagagactggtactaccactctcgtgtaattctagaacttgaagacaattctaggacagcagctttaagaatgtttatcattgcttaattagctttcctttatgcttttgtcatgttataaacaatttcgtttaattttaatattaatcgcagttaattttatacatgtgctctgttatttgatatcagttccgcataatggacaggctacaatacccttcagtagggtcacaaacgtgacccaggaagctgtattgcaccctgct

SEQ ID NO: 63 - DNA, 500 bp, Carrot cryptic virus
  source 1..500: mol_type = other DNA
gaaccgttcaccaccacccgctatccgccgcgtcgcgaaatccctgacgccctgtcaattcaattttctcatgctgaccatgagcttgaagaacaggccgaacagtatgctatgctcacccataccaacatcaaatggtaccagaacctggaaacccagaacaactggactgccattgctgctggagggctttaccttggagactactggaacatgatgccttataggttctctgctcctcttcactacaagtctcagtatgcccagatcatcgcctctcgctaccaccaacaagctgccaacaaagccgaataggcttaatgactcttacaaagtcgatcgcgcacaattttctccgtttttaaacggactatctgatcagaaaggattttttttttgtttaatctatttatatcttttgcaaatttctatctccttcttatctgtactactaagtacaaattttgttccctttccttctacctagaagactagttagt

SEQ ID NO: 64 - DNA, 500 bp, Crimson clover cryptic virus 2
  source 1..500: mol_type = other DNA
tcaactatgctcaatcctttattggatttaaaactgttgatgccactaggaatgatgacgtcctggatgctgtccctggaatgcccctgaataacctgattctctggtctccttatacctacactgcttatgaggatgaaacctatcctgatcccgttttcagtaattcccggcactactacttgaccaacctccgtaccatctttggtactgacaccaatttgatcaaggccgtccacccctatgaagcctaccccgtctcttaagtgattactccttttttcctttatttattaaagtttctgtttaatctattttcttttgatgacaatcacgtgtatgctacgtgattcttttgtttcgttttatttaatgaacttttctgtttgttttttttagtcgttattttctttaaagaaaaaaaaaaaaaaaaaaaaaaaaaaaaaaaaaaaaaaaaaaaaaaaattaaaaataaaacaaaaacccaaaaaaagttcccc

SEQ ID NO: 65 - DNA, 500 bp, Beet cryptic virus 2
  source 1..500: mol_type = other DNA
tactacctcaacacaagcgaagaacaagcgcaagaaagatgctgcaccagagcagttatctaccggacaaggagatggatcatcatcatcgaaagatgctgagatgtctactgaagaatcagaagctccaccaccgccgaagattccaagaccaagacaaatccatcaattcagattgatcgactgggtttaccactcaagagtaatcctaggagctgacaaagctatgcaacgacgtgccctttgggacttcattcatgctggagcaactccaagtcatcaaccaaggccttagtttatttgttttatattttaattaaatgtaatgaacaatgcaacttttggtttatttatcagaattttcagtttgatgtttaacttatcattgttatttatcacagtatctaacacactggtagctaaccgcactgatgaacagataaagggcagcaggcatgtacacccacaggaaattcctgagggactatacagccctgtct

SEQ ID NO: 66 - DNA, 500 bp, Beet cryptic virus 2
  source 1..500: mol_type = other DNA
gcagagaaactacactgtcaattttcctgagcttctaaagccaagagacaccggagcacgggctctagtcaccaccacccagagagcttcaacttctgggtctgttgaaccgacgacaggtgctcaaccagaacctgagacaccaacttttcagataacatttcgacaatttcgcattctcgattactgctatctagcgaaggtcatccacaagtctaattcccagatgatcaacaaggcactccgcaacttcatccagaagggagcctccaagaagaccgagtaactaatgtaataaattagattagttaaaactctgtcatattttgtgaacattttgaccattttgtgaacattttgacctacttttatcaatgaaagttttatttcaagaatttgtgtgtttaactacccactgcttgttgctttctgcataaaagggcagcaggcataagaaacccattagattaatttctaacaggactcttatggcccatgcc

SEQ ID NO: 67 - DNA, 500 bp, unidentified
  misc_feature 1..500: note = unclassified Partitiviridae
  source 1..500: mol_type = other DNA
gccgaccctgcctccctctttccattcgctattaagctcgaaaccgcatctcgagctcttcctgaattagctgaagcgatggctgcacttgcgcagaccaacgtgaatatctacccgacccatccctactacaaccacattggtgctgagtcgcgccgtggaccgttctggaaccttgctcccgttgaatcctccatcaccgatcactcgagctatctctcactccgagaggttgtgcgcggtttaatgaagcccaaaggctaattaaacttgccggttttcatgtcttcttgtttgaagttctgattttccgtttttccttttttgttgaaccattaattgaaaacaaaaaaaaatatttcctttgtttaattaaggttttgaagtaattctcttcccttttcgattacctcgtttttcgttcttagtacttttatttatccttttattttctacttaaagaaaaaaaaccaaaaaaaaaaaaaaaaaaaaaaaaaaaa

SEQ ID NO: 68 - DNA, 500 bp, unidentified
  misc_feature 1..500: note = unclassified Partitiviridae
  source 1..500: mol_type = other DNA
ggaacaaggtttaattagatataacctacggcaaggatttaaacggagaagaactgatccttcaacctccaatgataacgacatgcctgtcgaggacattgaggagccaccacaacctgaacaaggcgaagaccccatccacccggaagaggaagccgccccaccagtagagccgcagcccgatccagctccagcaccagcccctgcaccgcaagcccccgctgagccgatcataacgatgaatcgcgttagaaggtttcgcatcatcacttactgttactacagacaagtgaccgataatgaagacctgcattctcgtctcgctgcattacgacaagtggattcactgattgcatgattaggaacacttagttttgtactgctttactttctttaaaatttcagtctttaattttagttaaatctatcttattgtactgcaatttcaattaatgaaggttttgtttggcctatataatggtcacaggctttaactaact

SEQ ID NO: 69 - DNA, 128 bp, synthetic construct
  misc_feature 1..128: note = synthetic construct
  source 1..128: mol_type = other DNA
tgattgttcgagagaattacaccccatattcaaagcgatgaacttaattttagcagtcaaaatagaaagtgaaatttagatattttaattcaaattattatctactctacacaaatctagtttcaaca

SEQ ID NO: 70 - DNA, 500 bp, White clover cryptic virus 1
  source 1..500: mol_type = other DNA
cttgtacacactttttgcaatcaaaatagaacgcaaatcttgactgctctacctcaacttaatacctactctacatggattacctaatcactgcatttaaccgaatcactcactggttcttaactccaactaatctcgaatacatcggatcctactcccttccccctgggcttcttcgcgtaaatgatgttgctgtcgcaaatcataaagctaccttagatcgctcgtttgacaagtatctctatgaacatgagataaatcttatcactaaagagtaccgccgatctcctattgatgaagactctatacttgaggacttcttctcaggtgacctaccttatttcgaaatccctttcgacgaacacgtcgaacgtggactggaatgtatggctgccgcctttcggccgcctagaccctgtcgccctgctcacatcttagatgtgaaacatggatacccttacaaatggaacgtcaatgccgaacctcccttctccactgat

SEQ ID NO: 71 - DNA, 500 bp, Vicia cryptic virus
  source 1..500: mol_type = other DNA
gatcaaaagagtaacagctcttgccacgcttttcgcaatcaaaatagaacacgaatctcagctgctctacctcaactttttacttactctacatggattacctcatttccgctttcaaccgaatcactcactggttcatcactccgaccaatttcgagtacatcgggtacttctcccttccccccggccttctccgtgtcaatgacgtcgctatcgcgaatcataaatgcacacttgaacgatctttccacacctatctctttgatcacgaaatcaaaagaataatgatagatcatcgccgctcagacatcacagaagattctattttagaagacttcttcgctggtgacttcccctacttcgaagtacctttcgacgaacacgtcgaatatggtttacagtgtatggcagacgccttccgtccgccccgaccttgccgccctgctcacatcttagatgttaagcacggttacccttacaaatggaacgtcaacgcagaa

SEQ ID NO: 72 - DNA, 500 bp, unidentified
  misc_feature 1..500: note = unclassified Partitiviridae
  source 1..500: mol_type = other DNA
gataatgatcaaccgaaaaggttgattttcattatctagaagtaaattttctattgtaaattttcattatgaaattaacattagaagttacttctacacactaccgtgtatagcgagtttataagatttcaaattttagagttatctaattgtttactatcttacatccgttatatacaggtatggagcatcgctttcgtggaatccctcgtggtcttatagaactcgaagaaatcccgacgcgacgcttacgcgaggagcgcgttgtacatatagacgcctggagctcaaaagctatagacgccatcgtaccgctctcacttcgcatcgaactagatggctgggcccgaagttattatacacttcaagcgcacattgattcaatcatgcaatatgatcgtccaaagctgccgcaacccaccaatgctgcgtggaacaccaccacacaacacgtccgaacacagttcgcacggatggataaagtacagactctgtcttac

SEQ ID NO: 73 - DNA, 500 bp, unidentified
  misc_feature 1..500: note = unclassified Partitiviridae
  source 1..500: mol_type = other DNA
gataatgatctattagagatcattatcataggttttagttagaaattctaggttgcttgtgtgtgaaaactatagtacctgttgagtatctaaccactacccttctgttttgcaggttaggtaaaatttttaagttttaacgcgaagatttttcaccgtaccagcgcgaaactagaagatggaccacgagttcaggaagattcgtgaaggtctcatagagatcggcacagtttcccttcgtgtccaaagagacgagtttaaggtcatcattgatgaatatgccgcagaagctgttttcaagtttgttccttcaactatgctctcacaactccagggttgggccagaagtgtttactcactagatcaacatgttgatgccatacttgcgtatcgtcgtcaaaagttgcctgagccgacagatgatgtttggaaccaaaccaaacaacacaccttgcaattgttccgaagattccccaagatcacaccgatctcgtacaagt

SEQ ID NO: 74 - DNA, 500 bp, unidentified
  misc_feature 1..500: note = unclassified Partitiviridae
  source 1..500: mol_type = other DNA
gataatgatccaccgaaaaggtgaattatcattatctcagaagtaaaattcttaagtaagtttcgttcaagaaattaacattaagaggtacttctgcacacatctttgtgtaacggttttataagaagcttttctttacagttatctgtattttaactatcttacatccgtgtcacagaggtatggagcatcgcttccgtggtattcctagaggtctcattgagctcgaagaaatcccgacgcgacgcttacgcgaggagtgcgtaatacatatagacgcctggagctcacaagcaatagacgccatagtaccgctctcactacgcaacgaactagatggctgggccagaagctactacacgcttcaagcacacgttgattcgctcatgcaatatgatcgaccaaagttgcaaccacccaccaataccgcgtggaatatcaccacacagtatattcggactgaatttgctaggatgaagaaagtgactgcattgtcttat

SEQ ID NO: 75 - DNA, 500 bp, Beet cryptic virus 1
  source 1..500: mol_type = other DNA
gatcaaaagagtaacagctcttgcccatatttttcaagatcaaaatagaaattgaatcttagctgctctacctcaacttgttacctactctaaatggattaccttacctccgcattcaacagaatcacacactggttcaacgtcccttccaacttggaatacatcggaaccttttcgctcccccctgggattctacgtgtcaacgaagttgctatttctaatcacaagaaaacgttggaacactctttcaacaagtacctttatgcacatgaaatcaaattaatcacccaagactatcgccgctctgacatagatgaagaatctatactagccgatttcttttccggtgacgtcgaaaagttcgaagtaccctttgacgaacacgtcgaaaccggtctacgctgcatggcagacgctttccgtccgcctagactttgccgccctgctcacatcttagatgtcaagcacggatatccttacaagtggaacgtcaacgctga

SEQ ID NO: 76 - DNA, 500 bp, unidentified
  misc_feature 1..500: note = unclassified Partitiviridae
  source 1..500: mol_type = other DNA
agaattttccccgaaacacataatcgcggaaccaagacttagcagttaaggtcgcaagatttcggtcgtgaaaatcatttcattacagccagtcttcatcatgagaagattgcgcaaccctttagctggttacgtttacacaggattcatctcggatcttgaagagacaaatcagcgacacacaaacgttattcgacgggaaaccgccgtaacgtacagggatgagtttgcattgcaatccatcctagatctggactacaggcagtacgagcaacatctacaaggttggtcgcgtagttattacacccctgagaagcatcaggaagcaattttacaatactcctatccagacataccagttcaagccattaaaatggacgtatacaacgattgtatcgcccaggttcagaacgaatttcgtagccttccaaacgtgagggcctatagcgtattaacccaattagatttggttaaatacaagtcatcatcggcggccggtt

SEQ ID NO: 77 - DNA, 500 bp, Fig cryptic virus
  source 1..500: mol_type = other DNA
ggattgtccggcaggaaatctacgcacagtgtgtgtcaagggctagatcattactttgggagctcagagaaagagttagatcaaaacagtccgcaacagaacattgtacgcgatccgccaaataatccgggtaggttttaacatggaagcaggtcttatagagattggaaacattccagaacgacacttacgtgacgaattcataatactggtcgaccaaccagcatatgactcagtgagacgaaatgctccacaagctgacatgcaagaaattgatggatgggctagatccttttacacagtagaaggcattatggcatctattatgcaattcagcaaaccactcattcatgaaccaaccgatccaatatggaacgacgttaagagagaaacactaatgaagataggtagtttgttcccacaagttcaatcattaccatttgaaggaggttttgaccacgtcccatttgaatcgagctcatcagccggctacggctatg

SEQ ID NO: 78 - DNA, 500 bp, unidentified
  misc_feature 1..500: note = unclassified Partitiviridae
  source 1..500: mol_type = other DNA
ggaattacccccccactctttgacaaagagcccagagtgaatctgggagtgtcaagaccagggtagaggagcataattctttatttactcaacactatagatggcgttgagatcaattacagggtatgagttccatgattttcaatctagtttagaattgttaaatcaaactcacatccatatagtcaggcgggagtctggcgttacctaccatgacgagttcgcactttgtgaactattggtagataatacaagattgtatgagcaagagctagaaggctggagtagaagttactacaccggtgaacaacacatgaaagctatattgcagtacagtttaccaaacacaccaattcaagacattgatgtcggttgctatcagcaggcaatgaccaatgttcaagaacggttaagtagccttccaatcgtgagggcattcgacgtattgacccaattggatcaagtaagtttcgagtcgtcttcggcggcaggttacgact

SEQ ID NO: 79 - DNA, 500 bp, unidentified
  misc_feature 1..500: note = unclassified Partitiviridae
  source 1..500: mol_type = other DNA
gatcaaagaataacagctcttgttacaatttttcggaatcaaaatagaacctgaaacttagctgctctatctcaacttgttacctactctaaatggattacctcacaaccgcattcaaccggattaccaactggtttaccaaaccagctaacttcgaattcgtaggactctactccctacccccaggccttctgcgtgtcaatgacactgcaatttctaaccataagaagacactggaacattctttcagcaaatatctttatgaagatgagattcatctcatcaccaaagaattccgccgatctgatattgatctagattcaattcttgacgatttcttctctggcgatgttgaaccattcgaaataccattcgacgaacacgtcgaaaccggtctacgctgcatggcagacgctttccgtccgccaagactttgccgtcctgttcacatccttgatgtgaaacatcactatccttacaaatggaacgtcaatgctgaa

SEQ ID NO: 80 - DNA, 500 bp, unidentified
  misc_feature 1..500: note = unclassified Partitiviridae
  source 1..500: mol_type = other DNA
agaattttcccctgataaattatttatccgtgttgaacttacagcttcacggcttatatcaggttggattattatattcaatacactcatctgtttacatacaagatgagacccagcattacaggttatgattacacaaatttcacacaggatttgcttaaatctgaccgtaagcacccacacgtcgtacgtcgcgagactgcgactacttacagagacgactttgctttcaaggaagtaatttctctcgataggttagcttacatacagagattggaaggatggtcacgaagttactatctaccagagaagcatttagaggctttactccagtatgctacacctaatgtcccatgcactgctttgaaccttaacgtttatcgtcaagctattcaagtagtagagaatgggcttcgtagccttcaaccagtgagggcctttgatgttctaactgaactcaatcaaatttcatacaagcagtcctcagctgcaggttacga

SEQ ID NO: 81 - DNA, 500 bp, unidentified
  misc_feature 1..500: note = unclassified Partitiviridae
  source 1..500: mol_type = other DNA
acaactcctgctcaagcttttgacaatcaaaatagaacgtcaatctcagttgctctatctcaacttgttacctactctacatggattacctcacttccgcatttaaccgaatcactcattggttcaccaaacccaccaatttagaatacattggagactactctacacctcctagtatcatccgcgtcaatgaagttgctattgctaaccacaagaaaacacttcgctacgcctttgaacagtatctgtacgatcacgaataccgtatgatcgttgatgaatatcgtcgctcagacatagaccaagagtctatcctcgccgactttttctctggtgatattgaaccctttgatatacctttcgacgaacacgtcgaacatggactacgctgtatggccaacgccttcagacctcctagaccttgccgccctgctcacatcctagatgtcaagcacggatacccttacaaatggaacgtcaacgcagaaccacccttctca

SEQ ID NO: 82 - DNA, 500 bp, Pepper cryptic virus 2
  source 1..500: mol_type = other DNA
agaattttccaagccgtttacttggacctagttatctaggtaggaaaccgcttgggttaaattttaattttatatttaatagataagagccaacatggcaatttatcgcatgttgaatggatatgttttcaccgcatttggtaatgatcttgagaaattggatcaaagacatatacatcacatcagacgagaagaagcgacaacatatcgcgacgagttcgctctaaaggaactcatggatttaagccctattttgtatgaacaattcttagaaggatggtcacggagttattacgaaggatcaaaacacttacaagcaattatacaatatggcatcccagacccagatccaggcttaatagataatgatatctataacaaggcaggatatgtggtattagaaagtttaggtagccttccacgtgtgagggcatttgacgtactcactgagttagactcagtacactatgagcaatcttcatccgctggctatgactacc

SEQ ID NO: 83 - DNA, 500 bp, unidentified
  misc_feature 1..500: note = unclassified Partitiviridae
  source 1..500: mol_type = other DNA
agaatttatccttacttgcccacacaagaggacgaggagtccgaggagcaagcttaagggtgaaatttactattattttatcgtgacacactattacagtatgcacaacatcaacaattatgagttcacagactttactgaagaagtagaagaaactggcagaacccatcatcacgtagttaggcgagagcctgaggttacatatgaagatagatttgcttcgcaagtcttaaggcataagtatccagtcttgtatgaaaacatcatttcaggatggtctagaagttactactctggagcagaacacatgaaatcaatcatgcaatacgctacaccaaatattaaactagaacgtttacacgacggcgtataccataaagccatcgccaacgttcgagaaagtttacgtagccttccaactgtgagggcattcaatgtattgaatgagttagacctcgttccattcgaatcgtcatcatcggcgggttacgactacacag

SEQ ID NO: 84 - DNA, 500 bp, Pepper cryptic virus 1
  source 1..500: mol_type = other DNA
agaatttcccccttgttccaaggcacctctcacgctccggtgccataaacaagagattgaaattaatcaatttatatacttacctcgttacgatggtaagaggcacacttgttggttatgactatacccaatttcagggtgatttagttaaatcaactcaccgacaccctcatgttgttcaccgcgaaatcgcaacaacatatgtcgatcagtatgcttatgagcatattgaaacattcagtagtttatatcctgaacttatccttaaaggatggtctaggagttactatctgcccgagaagcatctcgctgcagtgttaaactacagtatgccaaatgttcctgcatctcagctcagtcagtcattatacagacaagcgattgaatcagctaagaatggatttattagccttccccgagtgaaggctttcgatgtacttaccgaaatggaccaagtaccttttaagtcgtcttcatcggcgggttataattacacaggc

SEQ ID NO: 85 - DNA, 500 bp, Carrot cryptic virus
  source 1..500: mol_type = other DNA
gatcaaaagatcaacagctacttccaaacgctttttgcaatcaaaatagaacgcgaatctcagctgctctaactcaacttgttacttactctaaatggattacctcacaaccgcattcaaccgaatcacacactggttcacaacacctaccgatttcgaatatattgggaacttctccctacctccaggcttgctccgtgtcaatgacactgcaatttctaatcataagaaaactctggaacattctttcagcaaatacctctgtgctgatgagattcatctcatcactaaagaataccgtcgatctgatatcgatcttgattcaattcttgacgacttcttctctggcgatgttgaaaaatttgaaattccattcgacgaacacgtcgaaacaggtttacgctgcatggctgacgccttccgtccgccaagactctgccgcccagctcacatccttgatgtgaagcatcactacccttacaagtggaacgtcaatgcag

SEQ ID NO: 86 - DNA, 500 bp, Beet cryptic virus 2
  source 1..500: mol_type = other DNA
agaattataccctagctttctagaaagacgggagtcatagtgaaagcgctagggtaagatttactactaattttattcgtcctacgtgttacttcaaacatgaggacaatcaacaactacgagtatacaagtttcaccgaagatctcgaggagacagattatactcacccacacgtggtcaggagagatcctgaagttacctatgaagacaccttcgcaaagaaggagttgttaagcaggtatccagcactgtatgaaaatttaatccgtggatggtcaagaagctattacactggacaagagcacttgcgtgcgatcatgcagtatgccacaccaaatactaactttagtgaatgtgtccaacacgcttataccaaagcgatcactaaagtgacagaaagtttacatagccttccaactgtgagggcattcaatgttctggaagagttagatctcattccatatgaatcgtcatcatcggcgggctatacgtatagagg

SEQ ID NO: 87 - DNA, 500 bp, unidentified
  misc_feature 1..500: note = unclassified Partitiviridae
  source 1..500: mol_type = other DNA
gaaaatcgcactacgtgcgtaaccaaaagggttgaattattttaaatataaaatatatccctccaacactacgatgttgagagatattagaaactttcaattaaccgaattcaattcagagttcgagttattgaatgacacacatccccatgtcattcggcgagaagctgaacagattttggtggatgagttcgctttaagcgaactaaacggtgcatggccatcgctatatgaccagaaactcaaaggttgggctagaagcttttataccttagagggtcatatgcaagcgatacatgcatattctaatccagatacacctatctcttctttaaacgagaccatctatcgagctacgattgatagtctaaagaatgagttaagtagccttccaactgcgagggcttttgatgtcttgactgagctagacaagatacactacgaacagtcctccgctgccggctacaattatattggacctaaaggaccactatatggtg

SEQ ID NO: 88 - DNA, 500 bp, Beet cryptic virus 3
  source 1..500: mol_type = other DNA
agaatttaaccaaaaccccatattgaggacgctgatttcattagcgccccgatatagttttacggtgaaatttataaatttagatttcaagtacgcactacatggcgtacagaaacattcgtgagtatgagttcactaacttcaatgaagaattgtaccagattgaaggaacccacactaatacgatcggacgagagtccgaggtaattctgaatgacgagttcgcgaaagctatcctcattgacgaatttccagtgctatacgagcaagtctgtcaaggatgggctagaagcttctacaccctagaggggcacatgcaagctattctcgcatacgcacaacccgacactccaagggaaacctttgaccaaaccatttgggatcaggcatacactgccgtccagaatgagttacgtagccttccaaaagcgagggcatttgatgttaatactgaacttgacaaagttccatatgagcagtcctcctcagcaggatatggc

SEQ ID NO: 89 - DNA, 92 bp, synthetic construct
  misc_feature 1..92: note = synthetic construct
  source 1..92: mol_type = other DNA
tggttttttactagccgatcattttatttatttcaatttaaattaaaaaataaaaagtgttaaataaatcttacctaacgcctccgaacttc

SEQ ID NO: 90 - DNA, 500 bp, White clover cryptic virus 1
  source 1..500: mol_type = other DNA
ccgatacatatttcaattcaattgtcaatgtcaagaagtctgaagttcgcaactcccttaatggatgcgaagtactctcctaccgtaaccacaacggcctacctcatcgtgatgagatcactatgctagcccagttctaccacactaaggctagagatcccacacctgagataaccatggcacaagccataggctttgcttacgccagttgcgccaatcacaaccgcgtactctgggtactagaagatgtatataattattatcgtgacttaggctacaggcctaatcgtgctggtttgactctcacctttggagactcaccggaccttaccatgcctgaaatgcctcttgatcattttccaactaaatctgaaattcggcgttaccacaccgaaacccactatcagaacgaagcacagaacgctcggacttggcctagaacattgtttatcaatgctccaggcgagtaaaaagccgatcgctttgtttgtttctttttt

SEQ ID NO: 91 - DNA, 500 bp, Vicia cryptic virus
  source 1..500: mol_type = other DNA
ccgagatccgtaatagactaaatggctgtgaagtactttcctaccgtaatcacaacggcctaccattccgcgatgagatcgcaatgctagctcagttctaccacactaaagctagagatcccactcccgaaattacaatggctcaagccataggcttcgcttacgcaagttgcgctacacatacacgcgtactttgggtcctcgaagatatatataactattatcgcgatcaaggatacacgcccaaccgtgctggcctcaccctcacattcggtgattctccagatctcactatgcctgagatgccccttgaccactttccaactaagtccgaaatagtgcgctacctaacatgcactaattatcggaatgaagcacaaaacgcccgcacttggccgagaacattgttcatcaatgctcccgccgagtaaagccgatcgctttgtttgtttctttttaattaaaataaaaaaaaaaaaaaaaaacaaaaaaaaattgcc

SEQ ID NO: 92 - DNA, 500 bp, unidentified
  misc_feature 1..500: note = unclassified Partitiviridae
  source 1..500: mol_type = other DNA
atacgttcagatggacaattttgaacccatctgtgctgaacacaattggacgattaacatcgccaaatctgctgtctcgcatgaagctgaaggggtatccttcttaagccgaaaagtccgtgagcactgccatgcccgtgacgaattactctgccttcgaatgctcaaatttccagaatacgtcgtagaatctggcgcaatgtcaactttgcgtgcacattccattcaccaagacgctggaatcaattcccgttacttgtattcaatatacaaataccttttacacaggtacggaaaagctgactccctgcctctgaaccaacagaactgggaccctctagagtacgagaacctacgtgtttcttttgctactcaaaactatgaataatgtaatgatgtttatcaatacactgtttgttgtcaaccaaccgaatattttatgtatttaaatttacatagaatgtgggttaggagcctgatcactcctaacccctctgatc

SEQ ID NO: 93 - DNA, 500 bp, unidentified
  misc_feature 1..500: note = unclassified Partitiviridae
  source 1..500: mol_type = other DNA
tcagtaccgctcaatacataccgctcgagaagttcggacccatatgcgaaccgtttatgtggagtatcaatactctaaagtctgaagtctcacgtgaaggtagacttactacctttctcagtcgatcgatccgcgataagcaaaactaccgtgacgaattcgtttgcctgcgtatgctcgtgtatcccgaatacgaagtggaagatggaagcatctcagccctccgtgccaaaagcatctatgtcgatgccggaatacattcccaatacttgtaccatgttttcttgtatttgaaacaaaaatatggattagccaataccctccctcacaacctgaggacctgggaccccacagaacatgaagctcttagagcttcttattctaatatcatgtaacactatttaataagttactctttctcaataaaagtttaaaatttaaattgtgctgcgtgtactattacagtagtaataagttcatgcagtacaataacaaatgtc

SEQ ID NO: 94 - DNA, 500 bp, unidentified
  misc_feature 1..500: note = unclassified Partitiviridae
  source 1..500: mol_type = other DNA
aaacttcaaacccatctgcgatgagaacctttggacgattaacatcgccaagtcagcaatctcacgcgaagctgaaggtgtatccttcttaagcaggaaagtccgcgaaatgtgccatgcacgtgacgaacttatctgtcttcgaatgctcaaatttccagaatacatcgtcgagacaggcgcaatgtcaaccttgcgagccttctcaattcataaagacgctggaatccattcccgttacttgtatcaaatctacaaattcctacttcataggtacggaaaggctgactcccttccgcttaaccagcagaactgggaccccatcgagtacgagaacctacgtgtttcttacgctactcagaattatgaataaaattattttatagacaataatctagttttcttcattgtcacttaaccgattgcacacctatctttaattaatagggtgcccccgggttaggagattagactttaagtctctcctaacccacctgatc

SEQ ID NO: 95 - DNA, 500 bp, Beet cryptic virus 1
  source 1..500: mol_type = other DNA
gaagtccgaattcggcaactcactcaatggtcgtgaagtcctatcctaccgcaatcacaatgggttcccccaccgtgacgagatcatgatgttggcccagttttaccacactaaagctagggacccaacacctgagatcaccatggcacaagccataggcttcgcctatgctagttgcgctaacaataaacgtgtcctctgggcacttaaggatgtttatgactactacaaagacttgggatacacccccaaccgagcaggcctaaccctgacattcggtgactcccctgatctttttgttcccgaaatctctcttgaacatttccccaccgagactgagatacgtcgttatttaacgagtacctcctatctcaatgaagcacagaacgctcgcacctggccgagaacgttgttcatcaacgctcccgctcagtaaaagccgatcgctttgtttgttttaaagaaaaaaaaaaaaaaaaaaaaaacaaatcaatttgtcc

SEQ ID NO: 96 - DNA, 500 bp, unidentified
  misc_feature 1..500: note = unclassified Partitiviridae
  source 1..500: mol_type = other DNA
ttgagtatctatacagactaattttaggacgaggtcccaagatatgtcatacattaggagacgattcactagtaggtgatgacgagttgatagtcccatcctcattcggccttatcgccaatcgtatcgggtggtacttcaatattgaaaagacggagtactcgagaataccagagatggtttcatttctaggtcgatcctacaaaggaggtctcaacgtacgagatcttaaacgatgtttaagattactagtctttcctgaataccctgtcccatcaggaagaatttcagcatacagagctaaatcgatagcagaagattgcggaggcatcagcgatctactaaatcgagtagcagcaagattaaagcgtcagtacggactcgcttctgaagaagaagttccaatctattttaagcgttatttaccttttatgtaatttacatttaagttatttaaagttttattaataagacgtggaggttacatttattttccctcc

SEQ ID NO: 97 - DNA, 500 bp, Fig cryptic virus
  source 1..500: mol_type = other DNA
aaccagatttaacacagcttcagatgcttggagagcagttcggatggacgctcaatattccaaagtgcagtcttacgcaagacagccaactggttactttcttaggaaggagtcagatgcatcaattaaacattagagagagacttaaagtactccgactgatgtgttttcccgagtacaaggttgaagaccctaaaatctcaaccactagggtaaaagcaatcgctcgagatgctggatggagtgatcctgtttataacaagatttacttacagctgaagcgcttgtatggtgaggtcgaaagacttccaccgcatttagcaacatttgttgaccgttttgattttcaagatgtaaacatgtgatgcaattttattaataaaagatactttcttttacgatatttcttagttgttgaattaacaattattatatatagtgagatgtgacgtgtccgaagttattatacaaatcggacaccaccacctcctggattaacc

SEQ ID NO: 98 - DNA, 500 bp, unidentified
  misc_feature 1..500: note = unclassified Partitiviridae
  source 1..500: mol_type = other DNA
cggctcaatcatcaacagattgcggatcgaatacatatggataaagcttagagggcacggaccgaccatttgttacacacaaggtgatgattcattatgtggtgacgatgaacggatcgaaccagagcgaatagctgatatcgccaatccaattggttggttgattaatccggcaaagacggcaacaacaaggtacccggaatacataacattcctaggaaggacatgttacggaggtttaaaccatcgagatcttatcagatgtctaaggttactaatctatccggaatatccagttccgtcaggcgctatctcagcgtatcgcgctaattcaattgctgaagactgcggaggcacgtcttcgatattgaatgatatcgcgcgacgattaacacgaaagtacggtcgtgtatctcatgaagaggtaccgaaagagctccgagtgtacagacactaggcagctcccgtaattttcgggaagggttcacataaccctgcct

SEQ ID NO: 99 - DNA, 500 bp, unidentified
  misc_feature 1..500: note = unclassified Partitiviridae
  source 1..500: mol_type = other DNA
tgaagttgcgaacacactaaatggacgcgaagtactctcttaccgcaaccacaacggtttccctcaccgtgacgagatcaacatgcttgctcagttctatcacactaaagcaagagatccgacacccgaaatcactatggctcaagcaataggttttgcctatgctagctgtggaaaccacaaacgcgtactttgggtactgaacgacatatacaactactacaaggacaaaggctacacacctaaccgtgcaggtctgtctctaacatttggagactcaccagatctgtttgtgcctgaaatccctcttgatcatttccctacaactaaggagatacgtcgctacctgacgtgttcttcttatgttaatgaagcacagaacgctcggacttggcctaggaccttatttcttaaggatcctgctgagtaaagcccgatcgctttgtttgttattttttaaaaaaaaaaaaaaaaaaaaaaaaaaacaaaaactaactgcc

SEQ ID NO: 100 - DNA, 500 bp, unidentified
  misc_feature 1..500: note = unclassified Partitiviridae
  source 1..500: mol_type = other DNA
caatcgtgaatcgactaaggatcgagtacatctggagaatcgcaacaggacatggtccgatacactgcgagactttaggagacgactctctctgcggagatgatatcttcgttccagctacacagttggccgatatcgcaaaccgcataggttggtacttcaatgccgataagacggaatactcaactataccagaaggcgtcacctttctaggacgaacgtcaaccggaaacttaaactcaagagatttaaccaaatgtcttcgactattagtttaccccgagtatcctgttacatcaggccgaatttccgcctatagagctaggtcaatagctgacgactcaggaggtctcagcgatttgcttaaccaagttgctattaggttagagcgtagttatggcattgcttcggaagaagaaattccagcttattttaaacgttatgtaccatttatgtaactttatttataaacgctttggaggctttacaaaccccctact

SEQ ID NO: 101 - DNA, 500 bp, unidentified
  misc_feature 1..500: note = unclassified Partitiviridae
  source 1..500: mol_type = other DNA
acgtactttaatgccaccatcaatgtcaagaagtcggaaatcagaaacacgctcaacggatgtgaagtcttatcatacaagaaccacaacggtttcccccaccgtgacgagatcactatgcttgctcaattctaccacaccaaagcacgtgacccaacgcctgaaattaccatggctcaagcaataggctttgcctatgccagctgcggcaacaacaaacgcgtactgtggattcttagagacatttacaacttttacaagatgcgtgacgtcacccctaatcgtgcaggtttaaccctcactttcggagactcaccagacgttttcatgcctgaaatccctcttgatcattttcctacaataactgaaattcggagatacctaacatgctctgaatatcgcaacgaagcacaagacgctagaacttggccgagatcactattcattagtggtcctgccgaataaagccgatcgctttgattgtttaaaattaaaaaaaa

SEQ ID NO: 102 - DNA, 500 bp, Pepper cryptic virus 2
  source 1..500: mol_type = other DNA
tctttagaaaggcatacgatgtcggaccaaagatgtgttatacacagggagatgattcactaatcggagtagactttcgagtagacccagacagattaagtgagatagcagcaccattgaattggaagcttaatcctgctaaaacagacgtttcgttgtacccggagcatgtcactttcttaggaaggactatgtacggaggaataaatcaaagagatcttaaaagatgtctgaggttattaatattccctgaatttccggttccatcgggagagatttcagcatacagagcagtatcaatcgcgcaagatgccggaggcacaagtgagatactgaacagtatagcaaagagacttcgaagacagtatggagtcgcagaggagcacgcagttccaaagcatttcaaactttacattccataaatgattgtaatatcaacctattaacaattcatatatgaaatgatgttgaagtcggagtggctgccaacacaccgctct

SEQ ID NO: 103 - DNA, 500 bp, unidentified
  misc_feature 1..500: note = unclassified Partitiviridae
  source 1..500: mol_type = other DNA
gatcattagtcaacagactacgcattgaatacatgtggattctcaaattcgatagaggtccaaagatatgttacactcaaggcgatgattcactgatcggtgacgatgaactcttcagtccaatggacatgacatttctggtcaagccatacaactggttaatcaacgtaaccaagtccatggcatccaagattccagaagcagttacgttccttggaaggacaagcataggcggcctaaatcagagagatctgaagcgctgcttacgactactgatattaccagagtaccctgttacatctggagacatatcagcatacagagctaaggcgatctggtacgactcggggttaacgagtgaagtgctaggcgaaatagctcgcgctcttcggcgtaagtatggactagcagagaaatccaaagttccaaagtatttaataccttggaaaggatgaactgcatccatacccacaccgggggaaggttctaacgaaccctcc

SEQ ID NO: 104 - DNA, 500 bp, Pepper cryptic virus 1
  source 1..500: mol_type = other DNA
acttgttggatcgattatcaattatttgagaatcaactacctctggagactcctcaccggacacccaccacaacagtgtcacactttaggagatgactcattggttggagataactcatatgttaatccacaagcgattgaagaagcagcaaacaagctaggctggcacttcaacccagataagactcaatactctacagtacctgaagagatcacatttctcggacgaacttacgtaggcggattaaataagagagatctaacgaaatgtattagattactcgtatatcccgaatacccagtagagtcaggtcgaatttcagcttatagagctaagtctatcgcacaagatgctggcggtctcagcgaagtgctaaataggatagcagataagcttagacgtatttacggtacggcttcagaagaagaggtaccaatttattttaaacgctatgtatttggagtttaaataaatacgtggaaggtatttaaaccttacc

SEQ ID NO: 105 - DNA, 500 bp, Carrot cryptic virus
  source 1..500: mol_type = other DNA
cttgccacgtactacttcaatgctattgtcaatgtcaagaagtccgaagtagccaatacacttaatggtcgtgaagtactctcttatcgcaatcacaacggtttccctcaccgtgatgagatcaacatgcttgctcaattctatcacactaaagcaagagataccacacctgagataaccatggctcaagcaataggtttcgcctatgctagctgtggcaaccacaaccgcgtattatgggtacttaatgacatatacaactactacaaggaccaaggatattcacctaatcgtgctggcctgactctcacctttggagactcacctgacctttttgttcctgaaatcccccttgaccattttcctacaaagaaggagatccgtcgttacctgacggcttcttcctacattaacgaagctcaaaacgctcggacttggcccagagacctgtttattaacaggcctgccgagtaaagccgatcgctttgtttgttattttc

SEQ ID NO: 106 - DNA, 500 bp, Beet cryptic virus 2
  source 1..500: mol_type = other DNA
cagtcgtgaatagattgagaattgaatatttatggaacctcaaattcaacagaggaccaaaagtatgtttcactcaaggcgacgactcgctaataggagatgacgaactgtacagcccaatggacatggcggcatttgttaaaccactgaactggtttataaacacatcaaagtcaatgacttcgaaagtgccagaagccataacgttcttgggacggtcaagtttaggcggcttaaatcaacgtgatttaaagcgttgcctccgtttactaatattaccagagtacccggttacatctggcgatatatcagcattcagggctaactcaatctggagagattctggttcaacaagccaaatccttcatgagatagcaaacgcacttcgacgaaagtatggaattgctaaagagcaggacgtaccaaggtatctcagaccctggaaagcctaatcagcatccatacctcaatcgggggggaaggttccaatggaaccctct

SEQ ID NO: 107 - DNA, 500 bp, unidentified
  misc_feature 1..500: note = unclassified Partitiviridae
  source 1..500: mol_type = other DNA
cgtattggatatttatggcgactaatgttcaatagagagcctaaactttgctacacgcaaggagacgactcattgattggtgatgacgaactaatccaaccagaaagactggctgaattagctgcaccattaaattggaacattaacccgaataagacagagtattctcgtactccagagtttatttcttttctaggacgaacatctagaggaggatttaacacaagagagcttgagaagtgcttacggttattaatctttcctgagcaccctgttccatcgggacggatttccgcatacagagcaaggtcgatcaatgaagacagtggctttaccagcgactatattcagaccgtcgctcgacgattaagaagacgctacggaatagctgaagaatcagaggttccgaagatgtttaaacgttacgttgttaggtaggtttaatgtatatcattattaagctttctatctaaaatgtaatagacttcttaacgggaggc

SEQ ID NO: 108 - DNA, 500 bp, Beet cryptic virus 3
  source 1..500: mol_type = other DNA
tcgaatatatatggagagtactattttcgagatcaccccatcggtgttacacccaaggtgacgattcattaatcggcgagacttttctcgttgaaccagaaacggtcgcccgcgaagcggcgaagtatggatggataatgaaccctgataagacagaatactctacagatccaggatatgtcacattcttaggaagaacggcacatggatttatgaacgctagatcactagacaaatgtctgagacttctaatgtttcctgagtaccctgtaacttcagggcggatttccgcctatagagcagagtcaattgcaagagattgcggaggacttagcgaggtaataaatctcgtagctcgtagattacgtagacaatacggagtagctagtgaggacgaagtcccacattactttaaacgttatgtagcttaattttgtttgtaatgaactcttttaataaacataataaagttctacggaaagggctatttaacccttacc

SEQ ID NO: 109 - DNA, 134 bp, Tobacco mosaic virus
  source 1..134: mol_type = other DNA
taggtttgagagagaagattacaaacgtgagagacggagggcccatggaacttacagaagaagtcgttgatgagttcatggaagatgtccctatgtcgatcaggcttgcaaagtttcgatctcgaaccggaaaa

SEQ ID NO: 110 - DNA, 198 bp, Tobacco mild green mosaic virus
  source 1..198: mol_type = other DNA
aaggtttgagggaacgtattttgagtgtgacagacggctcgccaattgaactcactgaaaaggttgttgaggagttcgtggatgaagtaccaatggctgtgaaactcgaaaaggttccggaaaacaaaaaagaaatggtaggtaataatgttaataataagaaaataaataacagtggtaagaagggttttaaaattg

SEQ ID NO: 111 - DNA, 133 bp, Tomato mosaic virus
  source 1..133: mol_type = other DNA
tgggtttgagggagaaagtaacgagtgtgaacgatggaggacccatggaactttcagaagaagttgttgatgagttcatggagaatgttccaatgtcggttagactcgcaaagtttcgaaccaaatcctcaaa

SEQ ID NO: 112 - DNA, 134 bp, Pepper mild mottle virus
  source 1..134: mol_type = other DNA
taggcttgagagagaaaattactagtgtgtcagaaggaggacccgttgaacttacagaagcagtcgttgatgagttcatcgaatcagttccaatggctgacagattacgtaaatttcgcaatcaatctaagaaa

SEQ ID NO: 113 - DNA, 139 bp, Paprika mild mottle virus
  source 1..139: mol_type = other DNA
aaggtttacgagagaaagtgactacagtgtcagaggacagcgctattgaacttacagaggaagttgttgatgagttcatagaggcagtaccgatggcacgtcggttgcaaaatttcaggaaacccaaaaataatattag

SEQ ID NO: 114 - DNA, 134 bp, Bell pepper mottle virus
  source 1..134: mol_type = other DNA
tgggtttgagggagaagattacaagagtaaccgacgccgggcctatggaacttactgaagaagttgttgatgagttcatagagaatgtcccgatggcggcaaggcttgcaagatttcgaaccaaatcctcaaaa

SEQ ID NO: 115 - DNA, 138 bp, Tomato mottle mosaic virus
  source 1..138: mol_type = other DNA
tgggtttgagggagaagattacaagagtggatgacgcaggtcccattgaacttaccgaagaagttgttgatgagttcatggagagtgtgcctatgtcagtcaggcttgctaaatttcgaaccaaatcctcaaaaagag

SEQ ID NO: 116 - DNA, 135 bp, Odontoglossum ringspot virus
  source 1..135: mol_type = other DNA
tcggactgagagagaagattttgaacgtaacagaaggagggcccaccgaacttactgaagcagttgttgatagattcgtggagaaagttcctatggctgctaggctcaagtcttttcgctctgtcaacaaaaaga

SEQ ID NO: 117 - DNA, 201 bp, Obuda pepper virus
  source 1..201: mol_type = other DNA
agggtctaagagagaaggtgactgccgtgtccgaagacgacgctatagaactcacagaagaggttgttgatgagtttatagaagccgtaccgatggcgcgacgtttgcagaacttgagaaaacccaagtacaacaaagaaaaagaaaataaaaatttgaataataaaaatagtataggagtttccaaacctgtcggtttag

SEQ ID NO: 118 - DNA, 136 bp, Rehmannia mosaic virus
  source 1..136: mol_type = other DNA
tgggtttgagggagaaaatcacaactgtgaaagatggaggacccatggaactcaccgaagaagtggtagacgagttcatggaagacgttccaatgtctgtcagacttgcgaagtttcgatctcgaaccggaaaaaa

SEQ ID NO: 119 - DNA, 134 bp, Brugmansia mild mottle virus
  source 1..134: mol_type = other DNA
aaggattgcgtgaaaggatcacgaatgttgccgaaggcggttcacttgagctcactgaagaagttgttgagagctttgtgaatgacgtccctatggcagtacgactgaaccatttacgtaatggaaacaaaaaa

SEQ ID NO: 120 - DNA, 134 bp, Yellow tailflower mild mottle virus
  source 1..134: mol_type = other DNA
ccggtctgagagagaaagtaacttccgtgtcagaagggtctccgattgaactcacagaagatgttgtagatgagtttttcgagagtgtccctatggctcgtaggttgcagaacttgaggaataggcgaggaaat

SEQ ID NO: 121 - DNA, 137 bp, Tropical soda apple mosaic virus
  source 1..137: mol_type = other DNA
ttggactgagggagaaaattacaaatgtctcagacggaggacccgtcgaacttacagaagaagttgttgatgagttcgtcgaatcagttcctatggctgagagattacgtaaatttcgaggacggtccaacaaaaag

SEQ ID NO: 122 - DNA, 432 bp, Cucumber green mottle mosaic virus
  source 1..432: mol_type = other DNA
taccctcgtctgtcgtagatattaattctagattcccagatgcgggtttttacgctttcctcaacggtcctgtgttgaggcctatcttcgtttcgcttctcagctccacggatacgcgtaatagggtcattgaggttgtagatcctagcaatcctacgactgctgagtcgcttaacgctgtaaagcgtactgatgacgcgtctacagccgctagggccgagatagataatttaatagagtctatttctaagggttttgatgtttacgatagggcttcatttgaagccgcgttttcggtagtctggtcagaggctaccacctcgaaagcttagtttcgagggtcttctgatggtggtgcacaccaaagtgcatagtgctttcccgttcacttaaatcgaacggtttgctcattggtttgcggaaacctctcac

SEQ ID NO: 123 - DNA, 347 bp, Cucumber fruit mottle mosaic virus
  source 1..347: mol_type = other DNA
tttttgtccttattatgctttgtatgatctgttggtgtcagcccaaggtggagccctgcaaacgcaaaatggtaaagacattttgcgtgactccataaatgggttgttaacgacagttgcgtctcctaggagtcggttccctgcggaagggttctttgtctggtcccgtgagtcgcgcattgctgctatattagattctctgctttcggcgttggattcaagaaatagggctattgaagttgaaaacccttctaatccttcgaccagtgaagctttgaatgctactaagcgcaatgacgacgcgtctactgctgcgcacaacgacattcctcagctgatttcagctt

SEQ ID NO: 124 - DNA, 297 bp, Kyuri green mottle mosaic virus
  source 1..297: mol_type = other DNA
tgttaacttctgttgcgtctctcaattcacgttttcctgctaatgagtttttcgtgtggtctcgtgagtcgcgcattgctgccataatcgattcgttgttatccgctttggattctagaaatagggctatcgaagttgaaaacccttctaatccttctaccggagaagctctgaatgcgaccaagcgcaatgacgacgcgtctacagccgcgcacaacgacattcctctgcttttagcagctctgaatgacggtgtgggcgtttttgatagtgcgtcttttgagtcagctttcggtc

SEQ ID NO: 125 - DNA, 335 bp, Zucchini green mottle mosaic virus
  source 1..335: mol_type = other DNA
tgctgtcttctgttgcgtcacccacttcacagtttccttccggtgtgttttatgtgtggtctcgtgagtcgcgcattgctgctttgatcgattctctcttcggtgctttggattcaagaaatagggctattgaagttgaaaacccttctaatccatcgactggcgaagctttgaacgcggttaagcgcaatgacgacgcgtctacagccgctcacaacgacattcctcagattttatctgctctgaatgaaggtgcgggcgtttttgatagagcgtcttttgaatctgcttttggtctcgtgtggaccgcaggttcgtctacctcgtcttgaggc

SEQ ID NO: 126 - DNA, 238 bp, Cucumber mottle virus
  source 1..238: mol_type = other DNA
tcttttcctcgcccgtgagtcctaccgtcagatttcctgacggagttttttatgtgtttttaggtaatcctgttttggatccgctttttaaagctctgttacagtcgttggatacaagaaatagggtcatagaggttgataacccttctaatcctacgactgctgagtctttaaacgctgtccagcgtactgacgattctactgtcagttcgcgtgtgggtttaataaacttgcgtgc

SEQ ID NO: 127 - DNA, 238 bp, unidentified
  source 1..238: mol_type = other DNA; note = unclassified Tobamovirus
  misc_feature 1..238: note = unclassified Tobamovirus
ttttcacctctcctgtgagtccgaccgttagatttcctgacggtccgtttttcattttcttagggaatcctgtgttggatccgctttttaaagctcttctgcagtcgctggatacaagaaatagggttattgaggttgacaatccttctaatcctactactgcggaatctttgaacgcgattcagcgtactgacgactcttccgtcagttcgcatgtcggtctagttaaccttcgcgc

SEQ ID NO: 128 - DNA, 246 bp, Turnip vein-clearing virus
  source 1..246: mol_type = other DNA
agggtttgagggaaaaggtcgtcgcaataaatgatccggacgtcgaaggtttcgaaggtgtggttgacgaattcgtcgattcggttgcagcatttaaagcggttgacaatttcaggaagaggaaaaagaaggttgaagaaagggatgtagtaagtaagtataaatatagaccggagaaatacgccggtcctgattcgtttaatttaaaagaagaaaatgtcttacaacattacaaacccgaatcag

SEQ ID NO: 129 - DNA, 246 bp, Ribgrass mosaic virus
  source 1..246: mol_type = other DNA
agggtttgagagaaaagatcatcgcgataagtgatccggacgtcgaaggtttcgaaggagtggttgacgatttcgttgattcggtcgaagcatttagagcggttgataatttcaggaaaaagaaaaggaagataggagaaaaggaagcggtaagtaggaataagtacagaccggagaaacacgccggtcctaattcgttatatattaaagaagaaaatgtcttacaacatcacgagctcgaatcag

SEQ ID NO: 130 - DNA, 246 bp, Wasabi mottle virus
  source 1..246: mol_type = other DNA
agggtttaagagaaagagtcatcgctgtagatgatccgaacgttgagggtttcgaaggtgtagttgacgaattcatcgattcggttgcagcatttaaagcggttgacaatttcaggaaaaagaaaaggaagattggaggaagggatgtgattagtaagtataagtatagaccagagaagtacgccggtcctgattcgttacttaataaagaagaaaatgtcatacaacatcacgaactcgaatcag

SEQ ID NO: 131 - DNA, 249 bp, Youcai mosaic virus
  source 1..249: mol_type = other DNA
aaggtttgagggaaaaagtcatcgctgtgaatgatccgaacgtcgaaggtttcgaaggtgtggttgacgatttcgtcgattcggttgctgcattcaaggcgattgacagtttcaggaagaaaaagaaaaggattggaggaagggatgtaaatagtaataagtatagatatagaccggagagatacgccggtcctgattcgttacaatataaagaagaaaatggtttacaacatcacgagctcgaatcag

SEQ ID NO: 132 - DNA, 258 bp, Streptocarpus flower break virus
  source 1..258: mol_type = other DNA
agggtttgcgtgaaaggcttaataatgtgggagatccaaatgttgaagtttcagaagttgtcgttgatgagttcgtggactcgatttccgcttcacagtcgttaagccgtgctcgtaataaatacgttcgtggtaatggtaaggttgggaataatagtggtggttttagtaagtataatcgtcatcagccggagaggttcgccggcaaggctatgtataattctaaaaatgtcgtacgtggtggtacttccgaaccaa

SEQ ID NO: 133 - DNA, 333 bp, Rattail cactus necrosis-associated virus
  source 1..333: mol_type = other DNA
tgcttttatgacaggatcttacccaacaattcgactgtgctcaacgactatgacagtttcaccttgagagtgagagacattcatctcaacttggacgattgtagactcgacctgtcaaaggacgatccagcaatctgtcggaggcagtcgggttatctgcgtcccgttcttagaaccggtacggagagacctcgacagcccgggctcattgagaatgtcttagctcttatcaagagaaatttcaatgcacctcatcttcttggaactgtagacgtcgatgcaatggccgacaccgtagtggataagttttttaaaattttcttccctgatggt

SEQ ID NO: 134 - protein, 159 aa, Tobacco mosaic virus
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 135 - protein, 159 aa, Tobacco mild green mosaic virus
MPYTINSPSQFVYLSSAYADPVQLINLCTNALGNQFQTQQARTTVQQQFADAWKPVPSMTVRFPASDFYVYRYNSTLDPLITALLNSFDTRNRIIEVDNQPAPNTTEIVNATQRVDDATVAIRASINNLANELVRGTGMFNQAGFETASGLVWTTTPAT

SEQ ID NO: 136 - protein, 159 aa, Tomato mosaic virus
MSYSITSPSQFVFLSSVWADPIELLNVCTNSLGNQFQTQQARTTVQQQFSEVWKPFPQSTVRFPGDVYKVYRYNAVLDPLITALLGAFDTRNRIIEVENQQSPTTAETLDATRRVDDATVAIRSAINNLVNELVRGTGLYNQNTFESMSGLVWTSAPAS

SEQ ID NO: 137 - protein, 157 aa, Pepper mild mottle virus
MAYTVSSANQLVYLGSVWADPLELQNLCTSALGNQFQTQQARTTVQQQFSDVWKTIPTATVRFPATGFKVFRYNAVLDSLVSALLGAFDTRNRIIEVENPQNPTTAETLDATRRVDDATVAIRASISNLMNELVRGTGMYNQALFESASGLTWATTP

SEQ ID NO: 138 - protein, 161 aa, Paprika mild mottle virus
MPYTVSSPNQLVYFGSVWADPIALIDLCTVSLGNQFQTQNARTTVQQQFSDLFKTVPTRTIRFSDGENGFRVFRYNSTLDPLITALLNSFDTRNRIIETENPANPNTAEIASATQRVDDATVSIRACINNLMNELARGTGMLNTASFETVSNLTWTTAATT

SEQ ID NO: 139 - protein, 160 aa, Bell pepper mottle virus
MSYTVNSPSQLVYLSSAWADPTDLLNLCTNSLGNQFQTQQARTTVQQQFSDVWKPTPQAGVRFPASGFKVYRYNAVLDPLITALLGTFDTRNRIIEVENQQNPTTAETLDATRRVDDATVAIRSAINNLANELVRGTGMYNQTSFESMAGLTWTTTPPSG

SEQ ID NO: 140 - protein, 159 aa, Tomato mottle mosaic virus [sequence omitted]

SEQ ID NO: 141 - protein, 158 aa, Odontoglossum ringspot virus
MSYTITDPSKLAYLSSAWADPNSLINLCTNSLGNQFQTQQARTTVQQQFADVWQPVPTLTSRFPAGAGHFRVYRYELILEPLITFLMGTFDTRNRIIEVRNPQNPTTTETLDATRRVDDATVAIRSAINNLLNELVRGNGMYNQVSFETMSGLTWTSS

SEQ ID NO: 142 - protein, 161 aa, Obuda pepper virus
MPYTVTSPSQLVYFGSVWADPITFIDLCTVALGNQFQTQNARTTVQQQFSDLFKTVPTRTNRFNDGENGFRVFRYNSTLDPLISALMNSFDTRNRIIEVDNPANPNTSEVASATQRVDDATVNIRACINNLMNELVRGTGMMNTASFETVSNLTWTTTTTT

SEQ ID NO: 143 - protein, 159 aa, Rehmannia mosaic virus
MSYTIATPSQFVYLSSAWADPVELINLCTNALGNQFQTQQARTVVQRQFSEVWKPSPQVTVRFPANDFKVYRYNAVLDPLVTALLGAFDTRNRIIEVENQANPTTAETLDATRRVDDATVAIRSAINNLVVELIRGTGSYNRSAFESSSGLVWTSGPAT

SEQ ID NO: 144 - protein, 159 aa, Brugmansia mild mottle virus
MAYTITAPNQIVYLGVAYADPILLMNLCTTSQGNQFQTQNARTTVQQQFADIWKAVPAVNIRFPENGFLVYRYNAVLDQLVTSLLNSFDTRNRIIEVENPPNPTNGEIMSATQRVDDATVNIRASITNLMNELVRGTGFLNRSSFESTSGLTWSTAPTT

SEQ ID NO: 145 - protein, 158 aa, Yellow tailflower mild mottle virus
MSYAITSPKQFAYLTNAYADPIALISLCNNALGNQFQTQNARTTVQQQFNDIWKELPTQISRFPVGIYRVYRFDSTIDPLITSVLNSFDTRNRIIETENPANPSTTEVVNATQRVDDATVNIRSSINNLCNELIRGTGFLNQATFEEASQLTWTARTT

SEQ ID NO: 146 - protein, 160 aa, Tropical soda apple mosaic virus
MSYTISNPNQLVYLASVWADPTELLNLCTSALGNQFQTQQARTTVQQQFSDVWKTVPATNIRFPNAGFKVYRYNAVLDSLITALLGSFDTRNRIIEVENPQNPTTAETLDATRRVDDATVAIRSSINNLMNELVRGTGMYNQAQFESVSGLTWATTATSS

SEQ ID NO: 147 - protein, 161 aa, Cucumber green mottle mosaic virus
MAYNPITPSKLIAFSASYVPVRTLLNFLVASQGTAFQTQAGRDSFRESLSALPSSVVDINSRFPDAGFYAFLNGPVLRPIFVSLLSSTDTRNRVIEVVDPSNPTTAESLNAVKRTDDASTAARAEIDNLIESISKGFDVYDRASFEAAFSVVWSEATTSKA

SEQ ID NO: 148 - protein, 162 aa, Cucumber fruit mottle mosaic virus
MSYSTSGLRSLPAYTKSFCPYYALYDLLVSAQGGALQTQNGKDILRDSINGLLTTVASPRSRFPAEGFFVWSRESRIAAILDSLLSALDSRNRAIEVENPSNPSTSEALNATKRNDDASTAAHNDIPQLISALNDGAGVFDRASFESQFGLVWTAASSSTSK

SEQ ID NO: 149 - protein, 161 aa, Kyuri green mottle mosaic virus
MSYSTSGIRSLPAFAKSFYPFYDVYNLLVSAQGGALQTQNGKDILRESLTGLLTSVASLNSRFPANEFFVWSRESRIAAIIDSLLSALDSRNRAIEVENPSNPSTGEALNATKRNDDASTAAHNDIPLLLAALNDGVGVFDSASFESAFGLTWTASATSSK

SEQ ID NO: 150 - protein, 161 aa, Zucchini green mottle mosaic virus
MPYSTSGIRSLPAFSKSFFPYLELYNLLITNQGAALQTQNGKDILRESLVGLLSSVASPTSQFPSGVFYVWSRESRIAALIDSLFGALDSRNRAIEVENPSNPSTGEALNAVKRNDDASTAAHNDIPQILSALNEGAGVFDRASFESAFGLVWTAGSSTSS

SEQ ID NO: 151 - protein, 162 aa, Cucumber mottle virus
MAYSPITPTNLSLFSSNYVPFTEFYNYLVTAQGEAFQTQQGRDSVRDSLSGFFSSPVSPTVRFPDGVFYVFLGNPVLDPLFKALLQSLDTRNRVIEVDNPSNPTTAESLNAVQRTDDSTVSSRVGLINLRAAITQGNGVVNRSIFESSNGLTWATSGSSSSK

SEQ ID NO: 152 - protein, 162 aa, unidentified
  REGION 1..162: note = unclassified Tobamovirus
MAYTPITPTNLPLFSSNYVPYTEFYNYLVSAQGEAFQTQQGRDAVRTTLSGFFTSPVSPTVRFPDGPFFIFLGNPVLDPLFKALLQSLDTRNRVIEVDNPSNPTTAESLNAIQRTDDSSVSSHVGLVNLRAAIAQGNGVVNRSIFESSSGLTWATSSASTSK

SEQ ID NO: 153 - protein, 157 aa, Turnip vein-clearing virus
MSYNITNPNQYQYFAAVWAEPIPMLNQCMSALSQSYQTQAARDTVRQQFSNLLSAVVTPSQRFPDTGSRVYVNSAVIKPLYEALMKSFDTRNRIIETEEESRPSASEVANATQRVDDATVAIRSQIQLLLSELSNGHGYMNRAEFEALLPWTTAPAT

SEQ ID NO: 154 - protein, 157 aa, Ribgrass mosaic virus
MSYNITSSNQYQYFAAVWAEPTPMLNQCVSALSQSFQTQAGRDTVRQQFSNLLSAIVAPNQRFPDTGFRVYVNSAVIKPLYEALMKSFDTKNRIIETEEESRPSASEVANATQRVDDATVAIRSQIQLLLNELSNGHGYMNRAEFEAILPWTTAPAT

SEQ ID NO: 155 - protein, 157 aa, Wasabi mottle virus
MSYNITNSNQYQFFAAVWAEPIAMLNQCISALSQSYQTQAARDTVRQQFSNLLSAIVTPNQRFPETGYRVYVNSAVLKPLYEALMKSFDTRNRIIETEEESRPSASEVANATQRVDDATVAIRSQIQLLLSELSSGHGLMNRAEFEVLIPWATAPAK

SEQ ID NO: 156 - protein, 157 aa, Youcai mosaic virus
MVYNITSSNQYQYFAAMWAEPTAMLNQCVSALSQSYQTQAARDTVRQQFSNLLSAIVTPNQRFPEAGYRVYINSAVLKPLYESLMKSFDTRNRIIETEEESRPSASEVANATQRVDDATVAIRSQIQLLLNELSNGHGLMNRAEFEVLLPWATAPAT

SEQ ID NO: 157 - protein, 157 aa, Streptocarpus flower break virus
MSYVVVLPNQLQFFTNAWADPIQLMNLCSASLSQIFQTQAARDAVREQFGNTLRSVVTQTTRFPEAGFQVYINDAALKPLWENLLKSFDTKNRILETEEETRPSTAEVLNATQRVDDSTTAIRGALQRLSDELNRGSGYMNRTSFETILLWSAATAK

SEQ ID NO: 158 - protein, 170 aa, Rattail cactus necrosis-associated virus
MPYINVQPKDFVYLTRSWVDPQRLIQFLSDMQTQAFQAQQSRTQLLNELSTMVVYGPTKSDRFPIDTYLINITKGGLSAYWFGLTQSADTKDRVFEVSEARAVTNAESKLATQRVDDATVAIRNAIKSTLSYLIAGEDIYSRTSFEAALGWIWQENLPPPPQTAASDSRT

SEQ ID NO: 159 - protein, 268 aa, Tobacco mosaic virus
MALVVKGKVNINEFIDLTKMEKILPSMFTPVKSVMCSKVDKIMVHENESLSEVNLLKGVKLIDSGYVCLAGLVVTGEWNLPDNCRGGVSVCLVDKRMERADEATLGSYYTAAAKKRFQFKVVPNYAITTQDAMKNVWQVLVNIRNVKMSAGFCPLSLEFVSVCIVYRNNIKLGLREKITNVRDGGPMELTEEVVDEFMEDVPMSIRLAKFRSRTGKKSDVRKGKNSSNDRSVPNKNYRNVKDFGGMSFKKNNLIDDDSEATVAESDSF

SEQ ID NO: 160 - protein, 256 aa, Tobacco mild green mosaic virus
MAVSLRDTVKISEFIDLSKQDEILPAFMTKVKSVRISTVDKIMAVKNDSLSDVDLLKGVKLVKKGYVCLADLVVSGEWNLPDNCRGGVSVCIVDKRMKRSKEATLGAYHAPACKKNFSFKLIPNYSITSEDAEKHPWQVLVNIKGVAMEEGYCPLSLEFVSICVVHKNNVRKGLRERILSVTDGSPIELTEKVVEEFVDEVPMAVKLEKVPENKKEMVGNNVNNKKINNSGKKGFKIEEIEDNVSDDESIASSSTF

SEQ ID NO: 161 - protein, 264 aa, Tomato mosaic virus
MALVVKGKVNINEFIDLSKSEKLLPSMFTPVKSVMVSKVDKIMVHENESLSEVNLLKGVKLIEGGYVCLVGLVVSGEWNLPDNCRGGVSVCMVDKRMERADEATLGSYYTAAAKKRFQFKVVPNYGITTKDAEKNIWQVLVNIKNVKMSAGYCPLSLEFVSVCIVYKNNIKLGLREKVTSVNDGGPMELSEEVVDEFMENVPMSVRLAKFRTKSSKRGPKNNNNLGKGRSGGRSKPKSFDEVEKEFDNLIEDEAETSVADSDSY

SEQ ID NO: 162 - protein, 257 aa, Pepper mild mottle virus
MALVVKDDVKISEFINLSAAEKFLPAVMTSVKTVRISKVDKVIAMENDSLSDVNLLKGVKLVKDGYVCLAGLVVSGEWNLPDNCRGGVSVCLVDKRMQRDDEATLGSYRTSAAKKRFAFKLIPNYSITTADAERKVWQVLVNIRGVAMEKGFCPLSLEFVSVCIVHKSNIKLGLREKITSVSEGGPVELTEAVVDEFIESVPMADRLRKFRNQSKKGSNKYVGKRNDNKGLNKEGKLFDKVRIGQNSESSDAESSSF

SEQ ID NO: 163 - protein, 269 aa, Paprika mild mottle virus
MSSKSIVNIEEFINMSKAEEVLPAALTRMKSVRISTVDKIMATDNDNISEVDLLKGVKLIKNGYVCLVGLVVSGEWNLPDNCRGGVSVCLIDKRMQRHNEATLGSYTAKASKKNFSFKLIPNYSITSQDAERRPWEVMVNIRGVAMEEGWCPLSLEFVSVCIVHKNNVRKGLREKVTTVSEDSAIELTEEVVDEFIEAVPMARRLQNFRKPKNNISKNFVNKGSSKPRNIPKLNEDKVFKLEKGLKGVKGMTNASRDDSSSEISSDSFN

SEQ ID NO: 164 - protein, 278 aa, Bell pepper mottle virus
MAIVVKDKVNVNDFINLSNSEKYLPSNSEKYLPSLMTPVKSVRISKVDKVLARENETLSEVDLLKGVKLIEGGYICLAGLVVTGEWNLPDNCTGGVSVCLVDKRMERASEATLGSYYTGAAKKRFQFKVTPNYSVTTDDAKKNIWQVLVNIKGVRMSAGFCPLSLEFVSVCIVFKNNIKLGLREKITRVTDAGPMELTEEVVDEFIENVPMAARLARFRTKSSKTGPKKFVRNSRDSKVRSNNLNRGVRPKFEEEGIVSDKLINDESESIDADSDSYL

SEQ ID NO: 165 - protein, 268 aa, Tomato mottle mosaic virus
MALTVSGKVRISEFIDLSKSERLLPSMFTHVKSVSVSKVDKVMVNEEDSLSEVNLLKGVKLIDGGYVCLAGLVVSGEWNLPDNCRGGVSICLVDKRMQRADEATLGSYYTGAAKKRFQFKIVPNYAITTKDAERNIWQVLVNIRNVKMAGGFCPLSLEFVSVCIVYKNNIKLGLREKITRVDDAGPIELTEEVVDEFMESVPMSVRLAKFRTKSSKRGPKHNSNNTNERKGRSNFRKKQDQESYGVSDSLDNLIEDDTETSVAGSDSY

SEQ ID NO: 166 - protein, 303 aa, Odontoglossum ringspot virus
MGRLRFVVLLSIFPIKTFSEPCYTMALVLRDSIKISEFINLSAFEKLLPSALTAVKSVRIPKVDKIISYENDTLSDIDLLKGVKLVENGYVCLAGLVVTGEWNLPDNCKGGVSICLVDKRMKRANEATLGSYHTSACKKRFTFKIIPNYSVTTADALKGIWQVMTNIRGVEMEKGFCPLSLEFVSICVVYLNNIKLGLREKILNVTEGGPTELTEAVVDRFVEKVPMAARLKSFRSVNKKKPSNSSKFVNGKSRLNSRNKLNYENGDSDVGTSVVDDIVVGNGVSDIRIDDDCESFDAQSESY

SEQ ID NO: 167 - protein, 274 aa, Obuda pepper virus
MSKAIVKIDEFIKLSKSEEVLPSAFTRMKSVRVSTVDKIMAKENDNISEVDLLKGVKLVKNGYVCLVGLVVSGEWNLPDNCRGGVSICLIDKRMQRHNEATLGSYTTKASKKNFSFKLIPNYSITSQDAERRPWEVMVNIRGVAMSEGWCPLSLEFVSVCIVHKNNVRKGLREKVTAVSEDDAIELTEEVVDEFIEAVPMARRLQNLRKPKYNKEKENKNLNNKNSIGVSKPVGLERNKVRSVVRKGVRSDSSLGVTDMSQDGSSSEISSDSFI

SEQ ID NO: 168 - protein, 267 aa, Rehmannia mosaic virus
MALVVKGKVNIGEFIDLTKTEKLLPSMFTPVKSVMCSKVDKVMVHENESLSEVNLLKGVKLIENGYVCLAGLVVTGEWNLPDNCRGGVSVCLVDKRMERADEATLASYYTAAAKKRFQFKVVPNYAITTHDAMKNVWQVLVNIRNVKMSAGFCPLSLEFVSVCIVYRNNIKLGLREKITTVKDGGPMELTEEVVDEFMEDVPMSVRLAKFRSRTGKKNNVKKVKFEGVDRSVPKMNKGKSRNNGGVFERNIFIDNDSETNVAEPDSF

SEQ ID NO: 169 - protein, 271 aa, Brugmansia mild mottle virus
MTLSIRDSFKIDEFINLSKVEKAVPALFSRVKTVRVSTVDKIMATKNDSLSDVDLLKGVKLVNNGYVCLVGLVVSGEWNLPDNCKGGVSVCLVDKRMKRANEATLGSYHSRACKKNFSFKLIPNYSITTADAEKQPWQVLVNIRGVQMEQGYCPLSLEFVSVCIVHKNNVRKGLRERITNVAEGGSLELTEEVVESFVNDVPMAVRLNHLRNGNKKEGFSKGKYVNNNAKERSVNKFVKKSVNGKGGLKVKEISTSVEDYNSSEVSSDSFI

SEQ ID NO: 170 - protein, 266 aa, Yellow tailflower mild mottle virus
MAIALRDTVKINEFINLSKTEEILPAMFTRMKSVRISTVDKVVAKENDSVSEVDLLKGVKLVKNGYVNLVGLVVSGEWNLPDNCRGGVSVCLVDRRMTRHSEATLGSYTAPACKKNFSFKLIPNYSVTTADAERRPWEVMVNIKGVAMAEGWCPLTLEFVCVCIVHKTNVRTGLREKVTSVSEGSPIELTEDVVDEFFESVPMARRLQNLRNRRGNINRNKSQVYKKKNKNNIGDALKERIDEVKDVENKRYDNDDYSIEESSDSY

SEQ ID NO: 171 - protein, 256 aa, Tropical soda apple mosaic virus
MALVVKDDVKISEFINLSTSEKFLPAAMTSVKSVRVSKIDKVIATENDSLSDVDLLKGVKLIEEGYVCLAGLVVSGEWNLPDNCRGGVSVCLVDKRMLRHDEATLGSYKTGAAKKRFAFKLIPNYSVTTADAKRNIWQVLVNIKGVAMEKGFCPLSLEFVSVCIVHKTNIKLGLREKITNVSDGGPVELTEEVVDEFVESVPMAERLRKFRGRSNKKSNKFVEKNNNKRGDKGGKLLKKIDNETETESSDAESSSY

SEQ ID NO: 172 - protein, 264 aa, Cucumber green mottle mosaic virus
MSLSKVSVENSLKPEKFVKISWVDKLLPNYFSILKYLSITDFSVVKAQSYESLVPVKLLRGVDLTKHLYVTLLGVVVSGVWNVPESCRGGATVALVDTRMHSVAEGTICKFSAPATVREFSVRFIPNYPVVAADALRDPWSLFVRLSNVGIKDGFHPLTLEVACLVATTNSIIKKGLRASVVESVVSSDQSIVLDSLSEKVEPFFDKVPISAAVMARDPSYRSRSQSVGGRGKRHSKPPNRRLDSASEESSSVSFEDGLQSDHT

SEQ ID NO: 173 - protein, 258 aa, Cucumber fruit mottle mosaic virus
MSISKVGVRNALKPEEFVKITWVDKLLPDAFTILKYLSITDYSVVQSKDYEHLIPVDLLRGVDFSKSKYVTLVGVVISGVWTIPENCAGGATVALVDTRMSLVSEGTICKFSVSAASRDFTVKLIPNYYVTAADASSKPWSLFVRISGVRIKDGFSPLTLEIASLVATTNSILKKGLRVSVIESVVGSDASVSLDTLSEKVQPFFDSVPITASVVSRDRSYVSKGRPPSRSGPVSRKSKSKSEAESFSDSGASEPLSS

SEQ ID NO: 174 - protein, 262 aa, Kyuri green mottle mosaic virus
MSVSSVGVKNALKPQEFVKLSWVDKVLPDMFTVFRYLSVTDYSVIKSRDSECLIPVDLLRGVDLSKSKYVTLVGVVISGVWTIPENCAGGATVALVDTRMSMVDEGTICKFSVAASTRDFMVKLIPNYYVTASDASSKPWSIFVRVSGVRIKEGFSPLTLEIASLVATTNSILKKGLRVSVLESVVGSDASINLDSVSDKVQPFFDSVPITAAVIARDRSYVSKSGPSRAVRPRPQSKSGKKGDANPAESMSDGDAYEPPRL

SEQ ID NO: 175 - protein, 261 aa, Zucchini green mottle mosaic virus
MSVSKLGVKNVLKPQEFVKLNWVDKVLPDMFTVFRYLSVTDYSVIKSRDSECLIPVDLLRGVDLSKSKYVTLVGVVISGVWTIPENCAGGATVALVDTRMSMVDEGTICKFSVAASARDFMVKLIPNYYVTATDASSKPWSIFVRVSGVRIKEGFSPLTLEIASLVATTNSILKKGLRVSVLESVVGSDASVNLESASEKVQPFFDSVPITAAVISRDRSYVSKSGFNRAVRSKPPSKGGKKFGDSAESLSEDSASELPGL

SEQ ID NO: 176 - protein, 262 aa, Cucumber mottle virus
MSISNLSVESQLKPSNFVKLSWVDKLLPDYFSILRFLSVTDHSVIKAREYESFLPVELLRGVDLTKHKYVTLLGVVISGVWTIPEGCSGGATVGLVDTRMERVVEGTVCKFSVPASVREFNVRFIPNYSITAADAARHPWSLFVRLKGVNIKDSFSPLTLEIAALVATTNSIIKKSLKAIVSDVVVGSDAAVAIADRDSQVNSFFDSVPITKSVVNFDKSYKSRVPKKDSSGGSGKVKRSTGTSGPADTEFSDDGLLSNHSD

SEQ ID NO: 177 - protein, 262 aa, unidentified
  REGION 1..262: note = unclassified Tobamovirus
MSISNLSVESQLKPSNFVKLSWVDRLLPDYFSILRFISVTDHSVVKAREYESFLPVELLRGVDLTKHKYVTLLGVVISGVWTIPEGCSGGATIGLVDTRMERVSEGTVCKFSVPASVREFNVRFIPNYSITASDAARHPWSLFVRLKGVSIKDSFSPLTLEIAALVATTNSIIKKSLKAVVSSVVVGSDAAVAIEDKDSSVNSFFDSVPITKSVINFDKSYKSKVPRKGSSVVSVKGKAGVGTSGPTDTEFSDYGLHSDHTD

SEQ ID NO: 178 - protein, 267 aa, Turnip vein-clearing virus
MSIVSYEPKVSDFLNLSKKEEILPKALTRLKTVSISTKDIISVKESETLCDIDLLINVPLDKYRYVGILGAVFTGEWLVPDFVKGGVTISVIDKRLVNSKECVIGTYRAAAKSKRFQFKLVPNYFVSTVDAKRKPWQVHVRIQDLKIEAGWQPLALEVVSVAMVTNNVVMKGLREKVVAINDPDVEGFEGVVDEFVDSVAAFKAVDNFRKRKKKVEERDVVSKYKYRPEKYAGPDSFNLKEENVLQHYKPESVPVLRSGVGRAHTNA

SEQ ID NO: 179 - protein, 267 aa, Ribgrass mosaic virus
MSMVSYEPKVNDFLFLTNKEKILPKALTRLKTVSISTKDIISVKESETLCDIDLLIDVPLDKYRYVGILGAVFTGEWLVPDFVKGGVTISVIDKRLVNSKECVIGTYRAAAKSKRFQFKLVPNYFVSTADAKRKPWQVHVRIQDVKIEAGWQPLALEVVSVAMVANNVVMKGLREKIIAISDPDVEGFEGVVDDFVDSVEAFRAVDNFRKKKRKIGEKEAVSRNKYRPEKHAGPNSLYIKEENVLQHHELESVPVLRSGVGRTHTNA

SEQ ID NO: 180 - protein, 267 aa, Wasabi mottle virus
MSTVSYKPKVSDFLSLSRTEEILPKALTRLKTVSVSTKDVISVKESESLCDIDLLVNVPLDKFRYVGILGVVFTGEWLIPDFVKGGVTVSVIDKRLENSKECIIGTYRAAAKDRRFQFKLVPNYFVSVTDAKRKPWQVHVRIQNLKIEAGWQPLALEVVSVAMVTNNVVVKGLRERVIAVDDPNVEGFEGVVDEFIDSVAAFKAVDNFRKKKRKIGGRDVISKYKYRPEKYAGPDSLLNKEENVIQHHELESVPVFCSGMGRAHSDA

SEQ ID NO: 181 - protein, 265 aa, Youcai mosaic virus
MSYEPKVSDFLALTKKEEILPKALTRLKTVSISTKDVISVKESESLCDIDLLVNVPLDKYRYVGVLGVVFTGEWLVPDFVKGGVTVSVIDKRLENSKECIIGTYRAAAKDRRFQFKLVPNYFVSVADAKRKPWQVHVRIQNLKIEAGWQPLALEVVSVAMVTNNVVVKGLREKVIAVNDPNVEGFEGVVDDFVDSVAAFKAIDSFRKKKKRIGGRDVNSNKYRYRPERYAGPDSLQYKEENGLQHHELESVPVFRSDVGRAHSDA

SEQ ID NO: 182 - protein, 288 aa, Streptocarpus flower break virus
MAIVLSKPKVSEFLNLTKAEEILPKFLTRLKTVAISTRDVVSVKGTTDLVDIDLLRDVPVNNWRYVGIVGIVASGEWLLPDNVSGGVAISFVDKRLVDSREAILGTYRAAAVEKRFQFKLIPNYFVSQEDALRRPWQVQVSLKGLKFEEGFSPLTLEFVSVVVCANSVVTKGLRERLNNVGDPNVEVSEVVVDEFVDSISASQSLSRARNKYVRGNGKVGNNSGGFSKYNRHQPERFAGKAMYNSKNVVRGGTSEPTAILHKRMGGSNSIDESLFSVAESDLSDSGCA

SEQ ID NO: 183 - protein, 334 aa, Rattail cactus necrosis-associated virus
MSSLCVSDPAVLLSPELHLKVPQLKSLVQFWKKPFKTCSISMSDVIKVASSNPLSIAVDFSSAMPSGPRNFKYVYILAVVLTGRWHLSESCPGGATFVLYDKRLCGRAEGIYGSFFSKVSASKFQVRFSVGHSMTVNDLLRNPLHLCFSLESVPCQEGYEPLSVEVSSLQLFTDCILEESLSAKLVKYPALCTDNMSVISNVDDVIFKFNSVLNVDNVPNSVKNVVKCNKVFKGRRSRGKWVGSQNEVVVKDGTNVTPHRLIAKDALHQRTTERLRLPDQVMGRPPASDTVSERYADSGFPSTAVSHTTSERTVDDGGLRSDQIGPFPYRYVPY

SEQ ID NO: 184 - DNA, 4759 bp, Arabidopsis thaliana
  source 1..4759: mol_type = other DNA
ttcattcatcatcttcctcctctctctcttctctgtatcacccaactaaatcctcacgggattagatccaaagtctcaaactttgatccaaaaacacaaactttgttcaaaagacataaactttgagccaaagtcacaagctttcttggtgaacgatggattacaatccaaatcttcctcctttaggaggaggtggtgttagtatgagaagaagcataagtcgaagtgtaagcagagcaagtaggaacattgaagatatcttctcatctggttcaagaagaacacaatcagtcaacgacgatgaagaagctcttaaatgggctgccattgagaagctaccaacttacagtcgtctccgaaccactctcatgaacgctgtagtcgaagacgatgtttacggtaaccagctcatgagcaaggaggttgatgtaaccaagcttgatggtgaagatcgtcagaagtttattgacatggttttcaaagtagctgagcaagataatgaaaggatcttgactaagctaagaaacaggatcgatagagttggtatcaaacttccaactgttgaagtcaggtacgagcatttgacgattaaagctgattgttacactggtaatagatctcttcctacacttttgaatgttgtgaggaacatgggagagtctgctttaggtatgattggtattcaatttgctaagaaagctcagcttacgattcttaaagatatctctggggttattaaacctggaaggatgacacttttgttgggtcctccttcttctggtaagaccactcttttgttggctttagctgggaaacttgataaatctctacaagtcagtggtgatattacttacaatggttaccaactcgatgagtttgttccgagaaagacctctgcttacattagtcagaacgatcttcatgttggtatcatgactgttaaggagactcttgacttctctgctaggtgtcaaggtgttggtactcgttatgatctgttgaatgagcttgcgaggagagaaaaggacgctggtatattcccggaagccgatgttgatctcttcatgaaagcttctgctgctcaaggtgttaagaacagtctcgtcactgattatactctcaaaattttggggcttgacatttgcaaagacacaatagttggagatgacatgatgagaggtatatctggaggtcagaagaaacgtgtcacaactggtgagatgattgttggacctactaagacactcttcatggacgaaatatccactggtcttgacagttccactactttccaaatcgtcaagtgtctgcaacaaatcgttcacctcaatgaagccacggtgctcatgtctctcctccagcctgctcctgagacttttgatttattcgatgatatcatcttggtgtcggaaggtcagatcgtgtaccaaggaccgagagacaacattcttgagttctttgagagctttgggttcaagtgtcctgagagaaaaggaacagctgatttcctgcaagaggttacttccaagaaagatcaagaacagtactgggtgaacccgaacagaccttatcactacattccggtttcagagtttgccagtagatacaagagtttccatgttgggacgaagatgtctaacgaacttgcagtaccgttcgataagtctcgcggccacaaagcagctcttgtgttcgataagtactctgtctcaaagagggagcttctcaagagctgttgggacaaagagtggctgcttatgcagcgaaacgcgttcttctatgttttcaagactgtccagatcgtcatcattgctgcaatcacgtctacactcttcctgagaaccgaaatgaacacaagaaacgagggtgatgctaatctctacataggagcattgctatttggaatgatcatcaacatgtttaatgggtttgcggagatggctatgatggtttcaagactccctgtgttctacaaacagagggatctcttgttttatccatcctggaccttctcacttcccactttcttgcttgggattccaagctcaatattagaatcgacggcttggatggtggtgacttattactccattggttttgcacctgacgccagccgcttcttcaagcagtttcttctggtgtttctgattcaacaaatggctgcatccctctttaggttgattgcttctgtgtgcagaaccatgatgattgctaatactggtggtgctctcactctacttcttgtgttcttgctcggaggcttccttcttccgaaaggcaagattcctgactggtggggttgggcttactgggtatctcctctcacctatgctttcaacggtctagtagtcaatgaaatgtttgctcccagatggatgaacaaaatggcttcttctaacagcacaataaagcttggaactatggtgcttaatacttgggatgtctaccatcaaaagaactggtactggatttcagttggagccttgctttgtttcacagccctcttcaacattctattcaccttggcacttacctatctcaaccctcttgggaagaaggcaggtttacttccagaagaagaaaatgaagacgctgatcaggggaaagatccaatgcgtagatctttgtctactgcagatgggaacagaagaggagaggtcgcaatggggagaatgagtagggactctgcggctgaagcatcaggtggtgcaggcaataagaaaggaatggttcttcctttcactcctttagctatgtcctttgacgacgtcaaatactttgttgacatgcctggggaaatgagagaccaaggagttacagaaacaagactccaactgcttaaaggtgtgactggtgcatttaggccaggagttttgactgcgcttatgggagtgagtggtgccggtaagactacgcttatggacgttttggccggaaggaaaacaggtggatacattgaaggagatgtgagaatatcaggattcccaaaggttcaagaaacatttgctagaatctcaggatattgtgagcagaccgatattcactccccgcaagtaacagtcagagaatctctgattttctctgctttccttcgtcttcctaaagaagtcggcaaagatgaaaaaatgatgtttgtggatcaagtgatggaattggtagagctggacagtcttagggactccattgttggtttaccgggtgtcacggggctttccacggagcagagaaagagactgacaatcgcggtggagcttgtagccaacccttccatcatctttatggatgagccaacttcagggctagacgctagagcagcggctattgtgatgagggcggtaaggaacacagtggacactggaagaaccgtggtctgcaccattcatcagcctagcattgatatctttgaagcatttgatgaattgatgctgatgaagagaggaggacaagtgatttacgcgggtccattgggtcaaaactctcacaaggtggttgagtactttgaatctttccccggagtgtccaagattccagaaaagtataacccggccacttggatgctcgaagctagctcactcgccgctgagctaaagcttagtgttgactttgctgagttatacaatcaatcagcattgcaccagcgaaacaaagcgttggtaaaagaactaagtgtaccaccagcaggagcatcagatctttactttgctacacaattctcacaaaacacatggggacagttcaaatcatgcttatggaaacaatggtggacgtattggagatctccagactacaatcttgtccgtttcatcttcacattggcaacatctctcttgattggtacagtcttctggcaaatcggaggtaacaggtcgaacgcaggggatctaacaatggtgataggagcattgtatgccgcgattatcttcgtgggaatcaacaactgttcaacagtacaaccgatggttgcagtggaaagaacagtgttctacagagaaagagcagcaggaatgtactcagccatgccatatgccatctctcaagtcacttgtgagcttccctatgtccttattcaaaccgtttactactcactcatcgtctacgccatggttggtttcgaatggaaagccgaaaagttcttctggttcgtcttcgttagctacttctcattcctctactggacttactacggcatgatgactgtttccctcacaccaaaccaacaagtcgcttcgattttcgcctcagcgttttacggtattttcaacctcttctctggtttcttcattccaagacccaaaatcccaaaatggtggatttggtactactggatctgccctgttgcatggaccgtgtatggattgatagtgtcgcagtacggtgatgtggagacacgtatccaagtccttggtggtgctcctgacttaaccgtcaagcaatacattgaggaccattatggtttccaatctgactttatgggaccagtggcggctgtactcatcgctttcaccgtcttcttcgccttcatcttcgccttctgcatcagaactctcaacttccagaccagataaaaccatctctcaaacaacaaagtctctttctttttttgtatcctctgtgttgtttatatgcactataagctttgtatgttctaaatatgtattgtagtgtcataacgaaatttttttgaatatttttatttattgtcttatagatgatgacgaatcggaatcatgtaatataattccacttttttttgtgttta

SEQ ID NO: 185 - DNA, 1642 bp, Arabidopsis thaliana
  source 1..1642: mol_type = other DNA
gcttcacgagacaaacgacgaacaacgcaattacgcgttcttatctttctgggtatttcttaattatattactctatataaataaaaatacccagaaacgtaatttcgtcgagttcaaaaattcaattcaaagaataatttctttccataaaaagattgttgtggattaacttctttcttaaaagaatctcatacccgtcgaatcttcttcttcaacctttttctgcgtttttaatattttccctacaaaaagattctcaaaatcgttttacataaacccgatcttgtcaatctccagaaacaaaaaaaaaaaaatggcgatcaagacaaagactagtctaattccaagttgttcatcacctgaggatctaaaacgcgttttgcaaacgctaggttcgagctggggagatgtagttgaagatctggagcgtcttgaggttgttccgttaaaaggagctatgactaacgaggtttatcagattaattggcctactttaaacggtgaagatgttcatcggaaagttcttgttcggatctacggtgacggcgttgaccttttctttaacagaggtgatgaaatcaaaacgtttgagtgtatgtctcatcatggttatggtcctaagcttcttggtcgtttctctgatggtcgtcttgaagaattcatacacgccagaactctttctgcagatgatcttcgtgtagcagaaacatctgatttcatcgctgcgaagctaagagagtttcataagcttgatatgcctggtccaaagaacgttcttctctgggaaagacttaggacttggcttaaggaggctaagaacttggcttcacctatagagatggataagtatcggttggaaggtttggaaaatgaaatcaatttgttggaagagagacttactcgggatgatcaagagattggtttctgtcacaacgatcttcagtatggaaatgtcatgattgatgaagtaaccaatgccattaccatcatagactatgagtattcgagtttcaacccaattgcttatgacattgcaaaccacttttgtgagatggctgctaattaccattcagacactcctcatgttctggactacactctatatccaggagaaggagaaaggagaaggttcattagcacatatcttggctctacagggaatgcaacaagtgataaagaagtagagaggttattgaaagatgcagagagctacactttagcaaaccatatcttctggggactatggggaatcatctcgggacatgtgaacaagattgagttcgattacatggaatatgcaaggcagagatttgaacagtactggcttagaaagcctttgcttcttgaaggatgaagcagattcgcattgaaaagccgatatacaactaaaagtttctgcaattaggtcaaaagaaaaaaagaaaatttagtagactatatgttgctattgttactctcatcagtttgtgtgtttcttcccttctatatagaggtttgttaactctcaaaacagttaaattgagtaaaacttgttctaaacaaagtattatatgataatagaagaagcaccagtggtctagtggcatgatagtaccctgccacggtacagacccgggttcaattcccggctggtgcaattta

SEQ ID NO: 186 - DNA, 1043 bp, Arabidopsis thaliana
  source 1..1043: mol_type = other DNA
agtttcaaattagtaaacgataaagtagatgataattaaattcatgtcatagccatgaacgaatgttatgttaaaaaaatttcatgtaagtaaattctttggagttcgattcttcttttgtccttacacaatcaaagatttttctttcaactttttttacttaacagcattaactctttctcagatcttcaattgttttcctaaacgaaattatcattaaattgtttaaaaaatacacatttaatataacatatatcataacaagatatattatatttatgaaaaagagatgtagtactacagcatatggtggcaaacaaacccactaaattcagtaaaatggaacccaccaaatttatctataaataagtccccaaacctctcccccaaaaatgcaacaacactcatctttcccaatataagtttttaccaaacaacaacttttgttttgcctttcctcaaaacataattttgtcttattttctaaaccggtaaaagatgtcgctagctatcgcagaagtttacacggtgaggaagtttcacagagagagtatgaagaaaccggccaaacagacggttaccggcgagggagatgaaaagattggcggaggaagtttcaaggaggcgccggtgaagtcaggtgggtgtagaagattcggacggtggttttttgggaagccgggaatgaagaaaagctctgctaaagtttcggatcccatggcgattgagtgatgaagaagaaattaaaacttttctcttctttgccttttttttttttctttctttattttgttttattttcattggtctttttttaatggctttttctagtgtcttctctttttctttattgggtgattttataaatagtcggtaaacattgtacaatatgaactacaaaaatatgagaaaatctatatatgtatgtagaatggatgtattccctaaataccacagctagtttgataagttttatgccaaaccaactaaacttgtatgccacgatatttcgtatttcagaaaatcgaaaattgttatttttt

SEQ ID NO: 187 - DNA, 1428 bp, Arabidopsis thaliana
  source 1..1428: mol_type = other DNA
acaaaaaccatttgaattttctttcttcttcaaaagttcgaaacttttttttttccggcaatggagggactagttccggtacagagacttccaattaaagtggcttcaccttctctgtatcggtgtaacaattctgtctcaatcagacgttcaatttcaggttttgccatggacaggaagataaactttagggctccgtcgcaattctcgatccgagctcaacagtcggatttgaaggaaagcttagctgtgtcttcttcatcagtagaggataaaggaaatgttcttagaatcaaggaatgggaagtagagatgtatcaggaggaattagctattagtcaaggtattaggataaggagaaaaccaccaagtaaggctcctttgggatactctggaccatttgagttgagattgcataacaatgatgctgattctcctcgtaatatcttggaggagatcacatggtacaaagacgtagaagtttcccggatgaaggagctaaatccgcttgacgtgctgaagaaagctgtagaggatgctcctcctactagggattttgttggggctcttaggatggctcataaaagaactggcttccctggcttgatagctgaggttaagaaggcttctccaagtagaggaatcttaaaagagaattttgacccggtcgagattgctcaagcttatgaaaaaggcggagcagcatgcctcagcgttttgacagaccagaagtatttccagggaggctttgaaaacttggaagcaataaggagcgctggtgtgaagtgtccactattatgcaaagagtttgttgtagatccatggcagatctactatgctcggactaaaggcgcagatgcagtactgcttattgctgctgtattggctgacctagaaataaccttcttgcttaagatttgcaagaagcttagcttggctgcccttgttgaggtacatgatgagagagagatgggtcgtgtacttggaatagaaggaatcgagcttgttggcatcaataaccgaagtcttgaaacatttgaagtggacatcagtaatacaaagaagcttcttgaaggagagcatggtagacaaatccgtgagagagacatgattgtggttggcgaatctggtctgtttacaccagatgacattgcctatgtacaagcagccggagtcaaagcagttttggttggagaatccattgtgaagcagaacgaccctgagaaaggaatagctggactttttggcaggaacatttctcatacttagagaccatatcataatacatctccaaatacgtttcctagtacatttgttttgtatcttgctctgtgagtattgaggaaagactagaaaggtttgttggttctgatgtttaagacattttcaaaaaaccatcgaataaactgaatcgaaatcgatttacag

SEQ ID NO: 188 - DNA, 1388 bp, Arabidopsis thaliana
  source 1..1388: mol_type = other DNA
agataagagcgaaacaaattacaaaattgaattacacaataatgtcctctaagcgacgactaccggtgaaatattttataagaaaacaaaaacaataattaacaaacaaaggcgacacctttagattcgacaagacgaccattttagaaaaagcttctcaaattctctccttatccccaaaaattcaaacttttttctctttggaaaattctgaggcaaaaatgcggcgtaattgctgccatgtttcatttgcttcaaccctcaagatccttaattttgtccaagctttcattggtgtttcgatcattatctactccatatggatgctccatgaatacagccgtcaccttcccgtggacccgcctccgtcagcttcatcaagctccggaaccgagatcgctactagtgtatctgagcccttgaagaatcctattgattttgtggcgagtatcattctcgggtctaatggtggtgatcatgggtttaacctacgctcgttggaccttcctgctccatggttcatctactctttcatggcggttggtatattggtctgcattgttaccttcattggtttcatagcagctgaagcaatcaatggctgttgcttgtgtttttactctattctcaaaactctgctcattctacttgaagctgcacttgttgcatacatcgctattgaccgtcactgggaaaaggatcttccgtatgatccaactggagaacttagtagtcttagggctttcattgaagaaaacatcgatatatgcaaatgggtggggattgctgtggtagcagtccagctactgtcattgctactagccatggttctcagagcgatggtttcaaccccgaaacccgaacttgacgaagaggaagatgatgagaacccaaggagtagaacttgggacccacttcttggtccgcaaggaaaccaagcgcctgctggctcaagcaagattgaaaactggagctcccgaatcagagaaaagtatggtttgaaccagagtccaccggtgaacccgaaaggttgatgaagccaaataatcatgtttctaaaactgcatgtgtccatatggtctctgtgaagctctctttctctatgttttgctctttgcaacttctcgagtgtgcataaacatttactcaaaacagtgctgtattgtgtttacacaaacggcataagcatcttcgtttgtgtgtttgcttgtacaaacaatcctgtgaacagtttgaagttagcatttccactataacgtttcgattcaatttataatgaagaaaacaaaactaaaaattgaattggacatcataaacagattaatgtaaaatgtatatgtaggttaaactctttcttagattatatcatctatatatgtttcattatt

SEQ ID NO: 189 - DNA, 1145 bp, Arabidopsis thaliana
  source 1..1145: mol_type = other DNA
gactccattattatccctctctctacaaaaaaaacagtagagagagatatagagagcatcaatggcgtcgtcgtcaagactttgcgattcgtgcaaatcgacggcagcaactctattctgccgcgcagacgccgcgtttctctgcggcgactgcgacggtaagatccacacagctaacaaactcgcatcacgtcacgaacgagtttggctatgtgaagtatgcgaacaagcaccagcacacgtcacgtgcaaagctgacgccgctgcgttatgcgtcacgtgcgaccgtgatatccactcagctaatccactctctcgccgccacgagcgcgtaccaatcactcctttctacgacgctgtaggtccagcaaaatccgcttcctcctccgtcaatttcgtagatgaagatggcggtgacgtcactgcttcgtggcttttagctaaagaaggaatcgaaatcactaatttgttctccgatcttgattatccgaagattgaggtaacgtcggaggagaatagctccggtaacgacggagttgttcctgtgcagaataagttgtttcttaacgaagattacttcaatttcgatctctctgcttccaaaatttctcaacaaggattcaatttcatcaaccaaactgtttcgacgagaacgatagatgtaccgttggtgcctgaaagtggaggagtgacggcggagatgacgaacacggagacgccagctgtgcagttatcaccggcggagagggaagcaagggttttgaggtatagagagaagaggaagaatcggaaatttgagaagacgattaggtatgcgtcgcgtaaagcttacgctgagatgaggccgagaatcaaaggacgttttgctaagagaacagattcgagagagaatgatggtggagacgtcggagtttatggcggattcggtgttgttccgagtttctgatattttccggtgaaagaaacatggtagtagtatatcggttaatagcagattaaggataattaaaactaaaatgtttcctgatttaatcaggggctaagttattgttagaagattgttgttttttgaattaattctcacaattgtatcctaaattatatagtttacttaatgttgaactaataaattttgatttacaatc

SEQ ID NO: 190 - DNA, 2268 bp, Arabidopsis thaliana
  source 1..2268: mol_type = other DNA
atggcggaatcaagctctcctctccctcctctttgtgaaaggatctcacacaaaagctatttcttaagagctgtggatctcactattctaggtcttctcctttctcttctattgtaccgtatcctacatgtgaaccaaaaggacaccgtttggatcgtggctttcctctgtgaaacttgtttcaccttcgtatggttgcttattaccaacataaaatggagtcctgctgattataaaacatatcctgaaagacttgatgaaagggttcatgaacttccaccggtagatatgttcgtgaccacagctgatccagtaagggagccaccgcttatcgttgtaaacacagtgctttcactgttagctgtgaattatccggcgaacaaactagcttgttatgtgtcggacgatggatgttcacctctcacttacttctctctcaaggaagcttctaagtttgccaagatttgggttcctttctgcaagaaatacaacgttagagttagagctccttttatgtacttcagaaactctccggaagcagcagaaggttctgaattcagtaaagactgggaaatgactaagagggagtacgagaagttaagccagaaagtagaagatgccactggaagttcccactggttggatgcagaagatgactttgaagctttcttaaacacaaaatcaaatgatcattcaactatagttaaggtggtatgggaaaacaagggaggtgtgggagacgaaaaagaggtccctcatgttgtttacatttcaagagagaaaagaccaaatcactttcatcattacaaagctggagccatgaactttctggtaagagtgtcagggttgatgacaaatgcaccatacatgttgaacgtagactgcgacatgtatgtcaatgaagcagatgtggtgcgacaagcaatgtgtatatttctgcaaaaatcaatggattcaaaccattgtgcttttgttcaataccctcaagatttctatgattctaatgtcggtgaactcaccgtcttacaattatacttgggacgaggaattgcgggaatccaaggacctcagtatgcgggttcaggatgcttccatactagaagagttatgtacggtctttctctagacgatttaggagacgatggaagtctttcttcaattgctacaaggaagtatttagctgaggagagtctaacgagagaatttgggaattctaaggagatggtgaaatcggtggtcgatgcgttacaaagaaaaccatttccccaaaaaaaccttaaggactctctagaaacggctcaagaaatgggacattgtcactacgagtaccaaaccagctggggaaaaaacatcggttggttatacgattcaacaacggaagatgtgaacacgagtatcggaattcattcgagagggtggactagctcatatatttttccggatccaccggcgtttcttgggtgtatgccacaaggaggaccggaggtaatggttcagcagcgacgatgggccacgggactgctcgaaatccttttcaacaaacaaagtccgttgatcggaatgttttgtcggaaaataagatttcgacaaagcttggcttatttatacgttttcagttggggtctaagatcaatccctgagctcttttactgtctcctgcctgcttattgcctactccacaactctgctttatttcccaagggagtttacttaggtataatcatcacacttgtggggatacattgtctttacactttatgggaattcatgaaccttggtttttccatacaatcgtggtatgtcactcaatcatttgggaggataaaaacaacttgtagttggttatttagcgtccttgatgtcatactcaagcttcttggaatatcgaaaaccgtcttcatagtcactaaaaagactatgcctgagaccaagtcagggtctggatccaaaaaatctcaacgagaagttgattgtccaaaccaagattccggtaaatttgaatttgatggctcgctttatttcttgccgggcacattcattgtgttagtgaatctagccgctctagccggttgtttggtgggtctacaaagtcgcggaggaggtggttcgggtttggcagaagcctgtggctgtattttggtggttattttgttccttccatttctaaagggtatgtttgagaagggaaaatatggtatcccattttctactctttctaaagctgcctttttagcagcgttatttgttgttttatctgtgggaaactag

SEQ ID NO: 191 - DNA, 1381 bp, Arabidopsis thaliana
  source 1..1381: mol_type = other DNA
catatttatactactagtatataagttaagggtttaagcctcttctcttccttctttctcttagccatcacatcgataatggcttctcttctaacaaaacccaaacccgttttcctctgttcaccatcgttatctccaagaactttgaacacagcaacaccgtcattgaatttcaccagaatttcattcacccatcaccaaaagcttgctcctttcaagcctcctagtctcgttgttgcattctctgaaaagggtttgaagagagatgtcaccacagctgctgcagcgacggagggagattacagaaggataatgttatctgatgtgttggtgaagaagaaggaaaaagtagtttggtgggagagagaatggaaagctatggactttggagctgttgctgtcgttttgtctatgcatttgcttagtcttttggctccgtttcaattcaattggagagctgtttcggttgcttttgggctttatatcgttacaggtcttctggggattactctgtctttccataggaatctttctcataaagccttcaagctacctaaatggcttgagtacttgtttgcttattgtggagctcaagctcttcagggaaacccaattgattgggtgagtacacataggtaccatcatcagttttgtgattcagacagagaccctcatagcccacttgatgggttttggttcagtcacatgaattggatgtttgataccaatacaatcacccaaaggtgtggagagcctaataatgttggggacttggagaagcagccattctatcgattccttcgaaccacctacattttgcatccgctggctctagcggttgctttatacgcaatgggtggctttccattcatcgtttggggaatgggtgtaagaatagtatgggtatatcatataacttggctagtgaactcagcttgtcatgtatggggaaaacaagcatggaacacaggcgatttgtctaagaacaactggtgggtagcagctctagcattcggggaaggatggcacaacaatcaccatgcttttgagttctcagctcgacacggcttagaatggtggcaacttgatatgacttggtacgtcgttaagttccttcaagccatcggtttagcaactgatgtcaagctcccatcggaagctcagaaacaaagaatggcattcaccagcgactgatcttaaaaaactacatggttatgcctaaagtcaaaactgtcggttcgattgaagctatgaaggttttaaaaaaggattttgttctgcttttttgtgtttacttaaaggtatatatctgtgcaagagattgtatgtgacaaactgattatgaataatatctataattacattcaatacaaaaggaagc

SEQ ID NO: 192 - DNA, 980 bp, Arabidopsis thaliana
  source 1..980: mol_type = other DNA
aaatcactcaccacataaagtaaaagtctcatctcttcataaaatagctttaaaccaatcaagtagagtttcttctcaagtctttttattttatttttatcaatggcttcttcagcagtgtctctccaatccatctctatgacaactctcaacaatctttcatgtaatcaacaatttcatcgaagctctcttcttggttcctctaaatccttccaaaatcttggtatctcatctaacggttcagatttctcctatccatcaagtttcactgccaagaagaacctcactgcttctcgagctctctcccaaaacgggaatatcgaaaaccctagaccaagcaaagttcaagaactgagtgtgtacgaaatcaacgaattagatcgacacagccccaagattcttaaaaacgcattcagcttaatgttcggtctcggagatctcgtaccattcacaaacaaactctacacaggcgatctcaagaaacgcgtgggcatcacggcaggtctctgcgtcgtcatcgaacacgtcccagagaagaaaggtgaaagattcgaagctacttatagcttctacttcggagactatggccacttgtccgttcaaggaccatacttgacttacgaggattcgttcctcgccatcactggtggtgctggaatctttgaaggtgcctacggacaggtcaagcttcaacagcttgtgtatccgacaaaactgttctacactttttaccttaaagggttggctaatgatttgccgttggagctcaccggaacaccggtaccgccgtctaaggacatagagccggcgccggaagctaaggcactggagcctagcggagttataagtaactataccaactaattcatgaaattaaaatgtttctcgtatcgctgtgtaatgatataaagatttgtagttttgtgctcttatctgtaataaaatcattgaggaataatatctcgagtatgttttaagtc

SEQ ID NO: 193 - DNA, 2706 bp, Arabidopsis thaliana
  source 1..2706: mol_type = other DNA
ccggttacagctacaccgccgttttctctctccgccgtcttttctcttcttctccgactgaatctgtgaaggtgaatttcggagttgagaaagagagattagcgtgtaatggcgatcgataatggcaagcttcaagaagaagaagcctcatctgttactcgattctataacattattttgggttgggactataaacagcttactaaagagaatgagaggaaaaatagaaaggattctaaagagaaattgaatgttgtaaagaatacttataaagatgttgatgattatttcgagacttttgaaccattgctcttcgaggaagttaaagctcaaattttgcagaataaagatggtgaagaagcatcggtgtgtaaaatgagattggtaatggagtgcaacgagggcgaagggtttcactttctgttagtcacatacgaacatgaggaggatgaatatcttgctcaaaatgatcttttattgctatcaaaagaagaggttaaagggaattcatttccttcatcttatgggtttgctgtagtggaacatcgtcaaaataatcttcttcgtcttcggatgtatttagctgaagacattgtgcagataaccaagaatactaaatcctctagaacaaagtcatttatccaagcgctttcaaatatgcgttctcttattacatcatctgccagccctattgacaaaagggtattcagtttaaagttatgtggtttatcaaccattattcgggaatatatagcactacgatcagttagttcgctccctttcaaggatttaatcttcactgcagcagaaaaatcttgtggttttggagatgaagcatggaaaatatctggacctctgaatgaattctttaacgaaaaccttaacaaatcccaaaaggaggcaattgatgttggtctatcaagaaaatccttcgtcttaatacagggtcctccaggtaccgggaaaactcagacgattcttagcatacttggtgctattatgcatgctactcccgcaagagtccagtctaagggtacagatcatgaagtaaagcgtggtattcagatgactatccaggaaaaatacaatcactggggaagagcctctccttggattttgggtgtcaaccctcgagatgctattatgcctgaagatggtgacgatgggttctttccaacctcaggaaatgaattgaaaccggaggtggttaatgcgagtcggaagtacagattacgtgtacttgtctgtgctccatcaaactctgcacttgatgagattgtgttgcggcttctgagttcgggtcttcgtgacgaaaacgcccagacatatacacccaaaatagtacgaattggtctcaaggctcatcactctgttgcgtcagtatcactggatcaccttgtggcacaaaagcgtggctctgccatcgataagccaaaacaagggactacaggaactgatatagatagtatccgcactgcaattttggaagaggctgcgattgtgttcgctacccttagcttcagtggctctgcacttcttgctaaatctaaccgtggtttcgatgtcgttataatagatgaagctgcgcaagctgttgagccagcaactcttatccctttggccactaggtgcaaacaagtatttctggtaggggatccaaagcaactcccagctactgtaatctcaactgttgctcaggattctgggtacggtacaagcatgttcgagagacttcaaaaggctggctatccagtaaaaatgttgaagacccaataccggatgcatccagagataagaagctttccgtcaaagcaattctatgaaggggcactagaagatggatctgacattgaagctcagactactcgtgactggcataaataccggtgctttggtccattttgcttttttgatatacatgaagggaaagagtctcaacatccaggagcaactggttctcgggtaaatctagacgaggtggaatttgttcttctcatctaccacagacttgtaaccatgtacccggagctgaagtctagttctcagctagctattatatcaccgtataactaccaagtaaaaactttcaaagatcggtttaaggagatgtttggtacagaggcagaaaaagtggttgatatcaacactgttgatgggttccagggaagggagaaggatgttgcaatattttcatgcgtcagagctaacgaaaatggacaaatcggttttctctctaattctcggcggatgaatgtggggattacacgagccaagtcttcagtattggtcgttggttcagctgcaacattgaagagcgatccactttggaaaaaccttatcgaaagtgctgagcaaagaaaccgattattcaaggtttcaaagccattgaacaatttcttcagcgaagagaatctggagacgatgaaactgacagaggacatggagattcctgatgcaccgttgtatgaagacgaatctcttccggtggcaccatatggtggagatgatgactttggagatggtgatgctgatcaagacgatgtagccatggccggagaagactaaactctaaaaaccttttcgttattagttagaactttgatattacaggaagtggttgaacatctaacaatatctacacttattatttgtatggttttattttggtttacatacagtaataagaatgtctttacttgtgctacg

SEQ ID NO: 194 - DNA, 1543 bp, Arabidopsis thaliana
  source 1..1543: mol_type = other DNA
ctctttctttgtgggttctagtttgtgtgtatatatactatatgcacatacacataggaagatatgatgggttacgagcagatgaatcagatgacaatgacgacaacagcgatgatgaagaattacaataagaagggactaatcaatactccacagaagaaaatgacgaggagaagtgtgtcggctattgatggtggtgcagctgcgacggctaaagaaggagatcgccgtcagaatatcaagactcttgatcttagcggcatgtccttggcttctctctctgcctcttctatcaacttagcttcaatctccaaactcgatctctccaacaacaatattcagaaaattccggaatctctagtagcgagaatgttaaatttgtgggcattggatttgcaatccaatcagctaaaaactcttccaaactccattggatgtctttccaaacttaagtttcttaatgtctccggaaattatctccaatctctccccaaaactatagaagattgcagatcgctggaagagctgaacgccaacttcaacgagcttacaaggctcccagacgcgatagggtttgagctaacgaatctgacgaagctctccgtcaactcaaacaagcttgttctcttgccaaactccgttagctacttgacgtcgctgcgtgtgctcgacgcgcggctaaaccgccttagttccctccctgaggatcttgagaacctcgtgaaccttcaggtcctcaacgttagccagaacttccagcacttgacgacgttaccttactccgttgggttgttgatatctctcgtggagcttgatgttagttataacggaattacggttttaccagactccctcggctgtcttcgccggattcagaagctatccgtggagggtaatcccctcatttccccaccatttgaagtggtggaacaggggttggaagcattgaagcagtatatgagcgagaagatgaccgagtcttataagaaaactccgaccaagaaaaagtcttggggcattggtaagctcgtaaagtacggcctgagctcttctccggggaggagtaccggccgtgaagacgggaaggaaggcttcatcaatgtatctgattaccgccaaatcgacggaatcgcctcaccgcgacatgtgtccctcttcaacccgcgacgcctcttgtctccactctccgcgtatttctctcctcctaggtattagcttatttaggtgtgttgtctgtctttttttttttcatcttctacatacgattccataccgttttcactgttacatccatgtagtgttaaattgttaatgttcttttacgttgttttctgcatgcttggtgtgaggataaaatttaattaaattgtgtatacgtttttttttccctgtctcatagaatttttctatggcttttctcatttcgttgtattgtagttcaattttgttgaacacatttgtaaactattacatagacacttaatattttactatgtatataatgtaaaatcgcatttttgaaagtactccaccatccttctca

SEQ ID NO: 195 - DNA, 1543 bp, Arabidopsis thaliana; identical to SEQ ID NO: 194
  source 1..1543: mol_type = other DNA

SEQ ID NO: 196 - DNA, 3675 bp, Arabidopsis thaliana
  source 1..3675: mol_type = other DNA
ctttcactcttcccttctctctccccaaaataaatcagaaacctatcaacattcttcaacagccacaagtaaaatttatagacatttctgtctgaagaagaaaaagattgaatttttttgcttgttgtgggttctttaatggatccaagaacaagacttgactatgctctgtttcagctcacaccaactagaacaagatgtgatcttgtgatattctctggtggtgagaatgagaaacttgcttctgggatttttcaacctttcgttacgcatctcaagagcgttagtgatcagatttctaaaggtggatactccgttactctccgtccttcctccgttggtgttccttggtttactaaagtgacccttcagagatttgtgcgatttgtgactactccggaggttcttgagaggtctgtaacattagagaaggagattgagcagatcgaggattcgattcaagctaatgcagccgccattgccggtgaagctgaaggaaacgagttgggtggtacatggacttctcaaaagtctacggctttatcaaagacgaaaggggaaactgatggagacactgtggaagaaaattcgaaggttggtcttcagcgtgttctggagaaccgtaaagcggctttgtgcaaagagcaagcgatggcttatgctcgagctttggttgttgggtttgaacttgactacatggatgacctcttttcgtttgctgatgctttcggagcttcgcggttaagggaagcgtgtgttaactttgtggacttgtgcaagagaaagaacgaggatagaatgtgggtggaccaaatcactgctatgcaagcatttcctaggcctgaattaacgttcatgggtgactctgggatcgtactcgctggtgaagagaatgatttgttgaatgctacaaacgtaaagcatggaaactctatggatgcctcgagccagggtagctttgagactggccaagaagggagagctcaaatggcaatgccgtggccaaaccagtttcctcagtacatgcagaactttcaaggacatgggtatccgcctccttatatgtttccggggatgcaaggtcaatcaccatatttccatgggaatatgcaatggcctgtaaacatgggagatgtggagtcaaatgagaagtcttccaagaagaaaaagaagaagaagaagaataagaagaagtcgaagcaagatgaatctgccgagccaagcgataattccagtactgagaccgaatcagaagacggaaatgaaggaaagaaacaatccaggaaagtggttatccgcaacataaattatataacttcaaagaggaatggagcgaaggagagtgattcagatgagtctggagaagaagaagggtttgtggatggagattctattaagcagcaagtagaagaagccattggctcagtggagagacgtcataaatcgacttcacaccgtcagaggaaacacaaaagtcacaacggtgatgatgattcaagtaacaaggaaactaaggggaatgataattgggatgccttccagaatcttcttttgaaagacaatgattcagagccagaagaattactacgtatatcatcaactgcgttaaatatggcgtctgaagttgtgagaaagagagaaccgccttctgatgattcattcttggttgcaattggaaatgaggattggggaagagaaaccagcattgagaaattcaatgcaggtgagaatgttcggataataaggaaagggaacaactatgacgaggagatgttgaatccgggtagaagcgatgaatcaagaagctattcgcaggcagagatgtcggttcatgatgggaagctcagaaccagaaatgaggcggaagaagactggttcatacgtaaccaagctggtccagaaacagatccaagtcttgtcaaaacatttgtaggagaccatttccacttaaataagagcagtgagagagatgtcctgaccgatgactctttcatgatccattctcgtgttgagaaccaagtagaagattctcggttaaggacagaaatcatggacttagacgtttacggaaccacccaacaagaaaacagtgccccggagaacactccgcatgaaccagatgacctatacatggttcttggacgggaacaagatgttaaacctacattgttaccatggacccctgaaattgactttgagactaacactttggctcaaagaacaagcagaatcgatttgataactgccacaaaggcctctgcaggtgaacaaaccttagatggtaaagaaaagaagagtcgtggtatctccaaggggaaggatgcaaagtcaagagcatcaagtagacctgacccggcgtcaaaagccaagagacctgcttggggaagtagagctgctgtctccaagtccaaatccgagatggaggaagagagaaagaagagaatggaggaacttttaattcagaggcaaaagagaattgcggagaagagttccggtggtagtgtatcaagttctttagcgtccaagaaaactcctactgtgaccaagtcagtgaagagctcgataaagaatgagaagactcctgaagccgcgcagtcaaaggctaagccagtactgagaagttccaccattgagcgccttgctgttgctcggaccgctccaaaggagccacagcaaaaaccagtaatcaaaagaacatcaaaaccttcaggatacaagacagagaaagctcaggaaaagaaatcgagtaaaataggtcaatcagatgcgaaaagtgtggaactttctcgtgacccgagtctcgaaatcaaggaaacggtggtggaagattcacattcttacttgtcagagaagcaagttgatgcacttcctgctgttgcttctgttgatgatttcaaagacattaaagaattgcatagcttgccatcagaagaaacggccagagtaaagaacagaccgaatgaaatcatagcagagaaagtacaagatcagacgaagatcgatgatcaggaaacagtgaagaatacatctgtttctgaagataagcaaatcaccacgaagcattactcagaggatgttggagaagtccaggcttcacaggaaaaaccggtgtctccaaagaagtcggtgactttttcagagacaaacatggaggagaagtattatttctcaccagcggtctcagagattgacatctcaacgccacctgctactgaagcagatcattcgaggaagaaatggaacagtgaagagacgtctcccaaggcaacggctaaagtcttcaggaagcttcttatgttcggaaggaaaaagtaaaaccgacgttggaattcttcttctccggcatcatcgaacctttgttttgagacgacgtcatgttgagttcagagaacaatatatggggatgtgttggaatcatgaatcgtaggttaaatcctacaagaggaaaagcttcaggagacagagaagatgaagagaagagaagaaaggaagaagaagaagaagtcgtgacaaagaaagtcagctaatagacatatctccgctatttaaagtcgagtctaagcctttatacacttaaaggttgagggttcgaacccttgttatgtctttttgccctaaaagaaaaaaactttctcatgaaattccgtgagatgattcttccattctaagcatttg

SEQ ID NO: 197 - DNA, 3675 bp, Arabidopsis thaliana; identical to SEQ ID NO: 196
  source 1..3675: mol_type = other DNA

SEQ ID NO: 198 - DNA, 3727 bp, Arabidopsis thaliana
  source 1..3727: mol_type = other DNA
atatccagataattaattctctcataatatcttctccacaatttcaactcttctaaagtctaaaccatgaagttactctcaaagacctttttgatattaactctcaccttcttcttctttggcattgcactagcgaaacagagctttgaaccagagatcgaagctttgaaatccttcaagaatggtatttccaacgaccctttaggagtattatcagattggaccatcatcggttcgttacgacactgtaattggaccggaatcacctgcgatagtaccggacatgtagtctcggtttccttgctggagaagcaacttgaaggtgttctgtctccagccatagcgaatctcacctatctccaggttcttgatctcacttcaaatagttttaccggcaaaataccggctgaaataggaaagttaaccgagcttaaccagcttattctgtacctaaactatttctctggttcgattccttctggaatctgggagcttaagaatattttctatcttgatcttagaaataatttgttgtccggtgatgttcctgaggaaatctgcaaaaccagttctttggtattgattgggtttgattacaacaacttaaccgggaaaataccagaatgcttaggagatttggttcatctccaaatgtttgtagcagctggtaaccatttaactggttcgattccggtatcaattggtactctggctaatttaacggatttagacctgagtggtaaccagttaaccggaaaaataccgagagattttggaaatctcttgaacttacagtctctcgttttaactgaaaacttgttggaaggagatataccagctgagatcggaaactgctcgagcttggtccaacttgagctttacgataaccagttaaccgggaaaataccagctgaattagggaatttggttcagctgcaagcactccggatatacaagaacaaacttacttcttcaattccatcttcattgttccggttaactcagttaacccatttggggttatcagaaaaccatttggttggaccgatatcagaagaaatcggttttcttgagtcacttgaagtcctcacacttcattccaacaacttcacaggagagtttccacagtccatcacaaacttgaggaacttgacagtcctaacggtggggttcaataatatttccggtgagctcccggcggatctagggcttcttacaaaccttcggaacctttcagcgcacgacaatcttcttaccggaccaataccttccagcataagtaactgcaccggtcttaaactcctggacctgtctcacaaccaaatgactggcgagatcccgcggggtttcggaaggatgaatcttacgttcatttctattgggaggaatcatttcaccggtgaaattccagatgatatcttcaactgttcaaacttggaaactcttagtgtggcagataacaacttaacaggaactctcaagccattaattgggaagcttcaaaaactcaggattttgcaagtttcatataactctctcactggaccgattcctcgagaaatcgggaatctgaaagatttgaatatcttgtaccttcactctaatggtttcacagggagaatcccgagagagatgtcgaatctcactctcctccaggggctaaggatgtattcaaatgatcttgaaggtccaattcctgaagaaatgtttgatatgaagctactctcagttcttgatctttccaacaacaaattctcaggtcaaattcctgccttgttctccaagcttgaatcgcttacctacttgagtcttcaaggaaacaaattcaacgggtctatccctgcaagccttaagtcgctttcgcttctcaacacattcgatatctccgacaatcttctcactggaaccatccctggagagctgttagcttctttgaaaaacatgcagctttacctcaacttctcaaacaacttgttgactggaaccatcccaaaggagcttggaaagcttgaaatggttcaagaaatcgacctttcaaacaatctcttttctgggtctattccaagatctttacaggcctgcaaaaatgtgttcacactggatttttcgcagaacaatctctcgggtcatataccagatgaagtcttccaaggcatggatatgatcataagcttgaacctttcaaggaacagtttctctggagaaatccctcagagcttcgggaacatgacgcatttggtctccttggatctctctagtaacaatctcactggtgaaattccagagagtctcgccaatctttcgactctgaaacatctcaaactagcttcaaacaacctcaaaggccatgttcctgaatccggggtgttcaaaaacatcaacgcctctgatctaatgggaaacacagatctctgtggtagcaagaagcctctcaagccatgtacgatcaagcagaagtcgagccacttctcgaagagaaccagagtcatcctgattattcttggatcagccgcggctcttcttcttgtcctgcttcttgttctgattctaacctgttgcaagaaaaaagaaaaaaagattgaaaattcatcagagtcctcattaccggatttggattcagctctgaaactgaagagatttgaaccaaaagagttggagcaagcaacagattcattcaacagtgccaacatcattggctcaagcagcttaagcacagtgtacaaaggtcagctagaagatgggacagtgattgcagtaaaagtattgaatctaaaggaattctctgcagaatcagacaagtggttctacacagaagctaaaacattgagccaactaaaacatcgaaacctggtgaagatcttagggtttgcgtgggaaagcggcaaaacgaaagctttagtgcttccatttatggagaatggaaacttggaggacaccattcacggctctgcagcaccgattgggtcgcttttagaaaaaatcgatctttgtgttcatatcgcaagcggaatcgattatcttcattctggatatggttttcccatcgttcattgtgatctgaagccagctaatatactccttgacagtgaccgcgttgctcacgtaagcgattttggaactgctcggattctaggtttccgcgaagatggaagcaccacagcttcaacatcagccttcgagggtacaattggatacttagctccagagtttgcttatatgaggaaagtgacaacaaaagccgatgtattcagcttcgggatcataatgatggagctgatgacgaaacagagaccaacttcgttgaatgatgaagattcacaagacatgactttgcgccaattggtggagaaatcgattggaaatggaagaaaagggatggttagggttcttgatatggaactcggggactctattgtttctctgaaacaggaagaggctattgaagactttctgaagctttgtttgttctgtacaagctctagacctgaagatcgacctgatatgaacgagattcttacacatctgatgaaacttagaggcaaagcgaattcatttcgagaagatcgtaacgaggatcgagaagtttagcagcaaagcttttttgatgacattttttcgactttgccaaactctgttacgaaggttgaatactttggacgtaaaacgttctgattttgcttttcgttatgtatttctgttttatataaggttgaaatttggttttgat

SEQ ID NO: 199 - DNA, 1177 bp, Arabidopsis thaliana
  source 1..1177: mol_type = other DNA
caaaacaaaacgccattttttgtcgattcttcgtcgtcctcctttgaaattcacaccaatttgtctctcttcctagagaaacaaaagattattcgaaactcgggaaagtatcatccggtcaccggaaaattgattatttccgatgccaataggtgaccgtcagaatccgagtgtggagaagaagaagaacctcttcaggctatgtccgttttggcagaggagaagcaccacttcttcatcttcaacacagaatccaaaccaaaactaccgtagccgccatggaaatcggaatacagatatttccgccgtctctaagcctccgttgactatgtcttccgtcgccagatcacttcttccagctcgccgtcgtctccgactagatccttctagttatctgtatttcccttatgaacctggaaaacaagttcgcagcgcaataaagcttaagaacactagcaaatctcatactgcatttaagttccaaacaacggcacctaagagttgttatatgcgtcctcccggaggtgttttggctccaggagagagtgtttttgctactgtgttcaagtttgtggaacatccagagaacaatgaaaagcagccgttgaatcagaagagcaaggttaagtttaagattatgagcctgaaagttaaaccaggagtagagtatgtgcctgagttgtttgatgagcaaaaggatcaagtagcagtggagcaggttcttcgggtcatttttattgatgcggaccgcccaagtgctgcattggagaaactgaaacgtcaattggatgaagctgaagctgcagttgaagcaagaaagaagcctcccccagagacagggcctcgagttgtcggggagggtcttgtcatagacgaatggaaggagagaagagagaagtaccttgctcggcaacaagtcgaatccgtagattctttgtcctaaacgcagagagcaatgtggacttgttctgcagatcatgtacataatttattatccctcgattatgtatttatctctagacgattactttgtggttcttgtatgtctctgtttgttgtgtgattcatgccggttttttctccttcatcaaattttgtttcggtcgtttttagttgatctagaatcgaaccagagtttgttcagtatttagttacaaatgaaaaatcctgttatacc

SEQ ID NO: 200 - DNA, 1115 bp, Arabidopsis thaliana
  source 1..1115: mol_type = other DNA
tactcacttttacaactaactcataagtaaaacacagacagtttttagatggctaagatatattccccttcttttcccggtactctctgtctttgcatttttactctcttgacgctaatgttcattcgggtttccgctcgacccgcgacattcgtcgaggatttcaaagccgcctggtcggaatctcacatccgtcaaatggaagacggaaaagctatccagctcgtccttgatcagagcactggatgtggatttgcttccaaaagaaaatatctattcggacgagtgagcatgaagatcaaactcattcccggagactctgccggtacggtcaccgctttctacatgaactccgatacggccacggtgagagacgagctagattttgagttcttgggaaacagaagtggtcaaccttactcagtgcaaacaaacatatttgctcatggcaaaggagatagagaacaaagagttaatctttggttcgacccatctatggattaccacacttacactatcttatggtcacacaaacacattgttttttacgtagacgatgtgccaataagagaatacaaaaacaacgaagccaagaacatagcttacccaacatcacaacctatgggagtatactcaacattatgggaagcagatgactgggcaacacgtggtggattagagaaaattgattggagcaaagctccattttatgcttattacaaagatttcgacatcgaaggttgtcctgttcctggaccaaccttttgtccatcgaaccctcataattggtgggaaggttatgcctatcagtctcttaacgccgttgaagctcgacgttaccggtgggttagagtaaaccatatggtttatgattattgtactgaccggtctaggtttcctgtcccaccacccgagtgtcgtgcttgaaaataattgcatacgtacgttgcaatgatcatgtttcgtcttgtacgagattttatatataaattgtgggaaaatgtggattgtattgtcctcttacgcaattgtgcagacgtacgtgagtgtgtatagatataattgtgatttgtggttatttgaaatttttataaacatagatgaattctgtgaag

SEQ ID NO: 201 - DNA, 1981 bp, Arabidopsis thaliana
  source 1..1981: mol_type = other DNA
actggtgtgcgcactaagcaagcatactctattatttgtcgaaatcacaagagccagccagtctctctgagttctatcacaatcttcttcttcagagcttattgagtaatggcggcttcaatgcagttctacggagttaaaacaccagaacttgccttaaactcaaaaaggattgagtttagctcaaaggggctcaacttctcagcactcgtgtcctctgctcgtgtattctctagaaacgtagatcgttcttgtaagaacatcgctctgagagttacttgtgaagcgggaagagtggagctgctagagagaaaagcctcagagactttcaagttaaacaaaactgagaaaaaattgacctgtgtaatgaaattcggtggctcatcagtggcatcagcagaaaggatgatacaagttgccaaactcatactcagcttcccagatgagaagcctgttgttgtgctatcagcaatggcaaagaccaccaataagcttttgatggctggggagaaggctgtttgctgcggtgtcaccaacgtcgacactatcgaagagttgagctatataaaggaactccatataaggactgctcatgagcttggagtggaaacagcagttattgcagaacatctagaaggactggaacagcttcttaaaggcgtcgcaatgatgaaggaattaactttacgctcaagagactacttggtttcatttggagaatgcatgtccactaggctatttgctgcgtacctgaacaaaattgggcacaaagcacgacagtatgatgcatttgagatcgggattatcaccacagatgacttcactaatgctgatattcttgaggcaacatacccggcggtttcgaaaaagttacttggtgactggagcaaggaaaatgcactccccgttgtcacaggcttccttggaaagggatggagatcttgtgctgtaactacgcttggtagaggaggtagcgatttgactgcaacaacgatcgggaaggctttaggtttgcgggagatccaggtttggaaagatgtggatggagttttgacttgtgatcctaacatttactgcggagctcaacctgttccacacttgacatttgatgaggctgctgaacttgcttactttggtgcccaggtgttgcatccactttccatgcgcccagcgcgagaaggtaacattcctgtaagggttaagaactcttacaatcccactgctccaggaactgtcatcaccagatcaagagacatgagcaaggctgtattgaccagcatcgttctgaaacgtaatgtcaccatgttggacatcactagcacccgtatgctcggccaatatggtttccttgccaaggtgttttccacatttgaaaagttgggcatatctgtggatgttgttgcaaccagcgaagttagcatttctttgacattggatccttcaaagttctgtagcagagagttaattcaacacgagcttgatcaggtggtggaggaacttgagaagattgctgttgtaaatctgcttcgacacagatcaattatctctctcatcggaaatgttcagagatcatcattcatattagagaagggcttccgagtacttcgaaccaatgggattaatgtccagatgatctcccaaggtgcatctaaggtaaacatctcactgatagtgaatgatgatgaggcggagcattgtgtgaaggctctccactcggccttctttgagacagacacttgtgaagcagtctcagaatgcccgaccggctacatcgctgcctcttcgtgaaacctctaggtgcaacctgcaaggtctgaaagtactagctagtttgatgttatgattcttgtcagactggtattaggaaacaaagctcccattctccttgtgtaatataatgtaatttgattgggagatgtactggaaaatcaataagttttcctatttgaatatatgattaggactgcctacaacatcagtt

SEQ ID NO: 202 - DNA, 3043 bp, Arabidopsis thaliana
  source 1..3043: mol_type = other DNA
atgagatctcttctctttgtactatcactcatttgcttttgctctcaaacagcactttcatggaagaaggaagagtttcgcagctgtgaccaaactccattttgtaaacgcgctcgatctcgtactcccggcgcgtgttctctaattgtcggcgatgtttccatcactgatggagatctcgtagcgaagcttctaccgaaagcgcctaatcaaggcgatggggatcagatcaagccgttgattctttctctctcagtttacaaggatgggatcgtgcggcttaaaatcgatgaggaccattcgttgaacccgccgaagaagaggttccaagttcctgatgtggtagtgtctgagtttgaggagaagaagatctggctgcagaaagtagcgacggagacgatctctggagacactagtccgtcttcagtagtttatgtatccgatggttacgaggcggtggtgcgacacgatccgtttgaggtgtatgtgcgtgagaaatcaggtgatcgccgtcgcgttgtgtcattgaattctcatggattatttgattttgagcagttggggaggaaaactgaaggagataactgggaagagaaatttaggactcatacagattctagaccatctggtcctcaatctattagtttcgatgtttcgttttatgattccagtttcgtttatggaattcctgaacacgccactagcttcgcgttgaagcctaccaagggtcctggagttgaggaatctgaaccctacaggctttttaatctagatgtgtttgaatacgatcatgaatcaccgtttgggctttacgggtcgattccgttcatggtttcgcatgggaagtctggtaaaacttcaggatttttctggttgaatgctgcggaaatgcagattgatgtgttggctaatggttgggatgcagagagtggtatttctttgccttctagtcacagtaggatcgacacattctggatgagcgaggcagggattgtggatacattctttttcgttgggcctgagccaaaggatgttgtaaagcagtatgcaagtgtgacaggtacttcagccatgcctcagttgtttgccactggttatcatcaatgtaggtggaactacaaagatgaggaggatgtggcacaggtggactcgaaattcgatgaacacgatattccttatgatgttctctggcttgacattgagcatacagatgggaagagatactttacatgggatagtgtgttgtttcctcatccagaggagatgcaaaagaaattggctgcaaagggtaggaagatggtgaccattgtggatcctcatatcaagagggatgactcatacttcttacacaaagaggctactcagatgggatactatgttaaggattcatctggaaaagactttgatggttggtgctggcctggttcatcatcttacattgatatgttgagcccagagattagaaaatggtggggtgggaggttctcgtataagaactatgttggttcaactccatcattgtacacctggaatgacatgaatgagccttctgtattcaatggtcccgaggttactatgccaagagatgcattacatgttgggggtgttgaacacagagaagttcataacgcatatggatattacttccacatggcgacttccgatggacttgttatgcgtgaagaaggaaaggataggccttttgtattgtcaagagcaatctttcccggcactcaaagatacggagcaatttggactggagataacacagccgaatgggaacaccttagagtctccattccaatgatattgacacttggtcttactggaattacattctctggagctgatattggtgggttttttggaaatcctgaaccagaacttctagttaggtggtaccaagtgggtgcttactatccatttttcaggggtcatgctcatcacgataccaaaagacgagagccttggttgtttggtgaacggaacacagaactcatgagagatgccatacacactcgttacacactgctcccatacttctacacgttgttcagagaagcaaacgttacgggtgttcctgttgtacgcccattatggatggaattcccgcaagatgaagctacttttagcaacgatgaagccttcatggtcggtagtggtctactggttcaaggagtttacaccaagggaacaacgcaagcttccgtgtatttgcctggcaaagaatcatggtatgacttgagaaacggtaagacttacgttggaggcaagactcacaagatggatgctccagaggagagtattcctgcgtttcaaaaggcaggaaccatcatcccaaggaaggaccggtttaggcgaagttcctctcaaatggacaatgatccttatactttggtggtagctttgaacagttctcaagaagcagaaggtgaactctacatcgatgacggcaaaagctttgaattcagacgaggctcttacatccatcgtcgcttcgtcttctcaaagggtgttcttacatcaacgaacttagctcctccagaagctcgtctctcttcccaatgcttgatcgacagaattatcctcttgggacacagctcaggtccaaaatctgcgttggtggaaccgttgaatcaaaaggcagagattgagatgggacctctgcgaatgggtgggcttgtagcttcctcgggtacaaaggtgttgactatccgcaaaccgggtgttcgagtggaccaagactggaccgtaaagattctgtgattgaacggtttgaaccagtttcactcatggccgttagagtggccgaaatctgcttttccggcgacggaatatcacactttttaatatatgtttggagatttagacttaaatagttgtaagagctaacagtttgaaagtcactttgcattgttgtttatcttcatataaatgagtttagattttgataatttcagaattcgtggaatcataattaacaattttgatagggaaaaataatttgttttttttagtcagagggtcaaataatcttaccttc

SEQ ID NO: 203 - DNA, 787 bp, Arabidopsis thaliana
  source 1..787: mol_type = other DNA
attttttctagggtttcctcttccgcattcgtcgtcttcagctcctcctacacagtttcttctctgaatctacttaggaagctaaaatgggtagatgtccgactagaaaagtgaagaagaggagattatctcacaaaacagctcgccgtgacaaattcgaagtcaaaggcgatgacttggtttacacagagttgcgtaaaccggagacggagattaagccgttacagcttgacgaggatttgcctggaatgggtcaattctactgcttacactgcgatcgatacttctccaacgtatcagtgagggatgatcatttcaagactaagaaacacaagaagcgtgtgaatatgatgatgggacaagcgccacactcgcaactcgatgcagatttagccgggggaatggggatgccggacaatggcccaaagctgatgtctaatttggtattcacagagttgcgtaaaccagagacggaggatttacctggaatgggtcaattcaactgcttactctgccatcggaacttctccaatgcatcagtgatggattatcatttcaagactaagaaacacaaaaagcgtgtgaagaagatagagcgaccagcgccacactcgcaactcgatgcagatttagctggtggaatgggaatgccagacaatggtccaaagctgatgtctgcctgagtttttacagttattttccttatttagttaatggaacccgaaattttgttgtaaaactataatctcctaaattaatgatacaggttttttgtctgttgccaagt

SEQ ID NO: 204 - DNA, 1397 bp, Arabidopsis thaliana
  source 1..1397: mol_type = other DNA
aaaataatggcttcgcccgggttcgaaccggagaccttcagtgtgttagactgacgtgataaccaactacaccacgaaacctacatgcttctttctaacctcaacaatatatatatacatatagataactgtttaatattgtaacttgtaaccagtgtcggctaatttcgacttttgtagatctttttctgctctttctctctctctctctctctctctctctctctctctctctcttgtattatttctatctcccccgccgtcgaaagagaaacgtcgatcggagaacctttgaaatgtcgactggattagatatgtctctcgacgacatgatcgccaagaaccgtaagtctcgtggtggagccggccccgctcgtggaaccggatccggatccggaccgggtccgactcgccgcaacaaccctaatcggaaatcaacccgatctgctccataccaatcagccaaggcgccggagtccacctggggtcacgacatgttctccgatagatctgaagatcaccgatcgggacgttcctccgccggaatcgaaactggaaccaagctctacatttccaatttggattacggtgtcatgaacgaagacatcaaggaactgtttgctgaagttggagaacttaaacgctacacagttcattttgatagaagtggaagatcaaagggaactgctgaagtagtgtattctcggcgtggcgatgcactcgcagctgtgaagaagtataatgatgttcagctggatggaaaacccatgaagatagagattgtgggcactaatcttcaaactgctgcagccccgtctggtagacctgcgaatggaaactccaatggtgctccatggagcagaggaggacaagggagaggaggtcaacaacgaggtggtggacgaggaggcggtggccgaggtggtggtggtcgtggtaggcgtcctggtaagggccctgcagagaagatttctgcggaagatcttgatgcggatcttgataagtaccattctggagatatggagacaaactaaggaacgtgactgatcttctcaaaccggtaggggttttaggaggaagagaatcgagaaaatgtttgccagaggctttaccacttagcgcctttttggctgtgttgttcatttgtttcattagaatgactttacagaattgagaatatgtgttatttaaagttgttgtctatcttaataccctcaagtgaaaggcagaggaaggaataccaatttcgtttacagatcatatgcaggcaaaagagttttcagaaatcaggtttagttacttgtattgtgtctcttatccaagccatgccttgtggtattatgagcgcagaattttgattgaatattgttcatttgcattaaagtatcaaaccattt

SEQ ID NO: 205 - DNA, 636 bp, Arabidopsis thaliana
  source 1..636: mol_type = other DNA
aaaactcctccgtaaaaccctttcatttgcatttgggaatcgattgctcttcttcgtagtcgcctttgaattcgcagagtgagaagcaatgaaggtgagatcatctgtgaagaagatgtgtgagttttgtaaaacagtgaaacgtcgtggacgtgtgtatgttatatgctcttccaatcctaagcacaagcaaagacaagggttcagttcctttgcttatgaaggaattacaccttcccctttgtttgctgagccgattgctagtcaggagttggtgaggctgcctggtcagggggtatcagccggactggcatcacttttacacaaaaggcctatgccaacagcgtatttcggatggaggtctggtcttgcatccattctcttcaaacaaggaaattaggatgtcagcatttccctttttattcttcagatttgtaatcgcagttttcttccaattcctatgaaacattattgtatatctctccacttttactttggtcagtcgtatgctccttcgagacctgtttgattgtgcctcgcatgacctgaacaaagagatatattatgacactcgagtatttgtttttacattttccactggagcatatgttagggatcaatatgtttttcttttag

SEQ ID NO: 206 - DNA, 666 bp, Arabidopsis thaliana
  source 1..666: mol_type = other DNA
tgctcttcttcgtagtcgcctttgaattcgcagttagagatggatgcttcagattcgttttacttttgcttccatcatcggatcaactaaggattgcagtttttctgagtgagaagcaatgaaggtgagatcatctgtgaagaagatgtgtgagttttgtaaaacagtgaaacgtcgtggacgtgtgtatgttatatgctcttccaatcctaagcacaagcaaagacaagggttcagttcctttgcttatgaaggaattacaccttcccctttgtttgctgagccgattgctagtcaggagttggtgaggctgcctggtcagggggtatcagccggactggcatcacttttacacaaaaggcctatgccaacagcgtatttcggatggaggtctggtcttgcatccattctcttcaaacaaggaaattaggatgtcagcatttccctttttattcttcagatttgtaatcgcagttttcttccaattcctatgaaacattattgtatatctctccacttttactttggtcagtcgtatgctccttcgagacctgtttgattgtgcctcgcatgacctgaacaaagagatatattatgacactcgagtatttgtttttacattttccactggagcatatgttagggatcaatatgtttttcttttag

SEQ ID NO: 207 - DNA, 1991 bp, Arabidopsis thaliana
  source 1..1991: mol_type = other DNA
attccttgcttcatttttcgtcaccctagccgctttactctcttgcgatatctctgaggtaattctggagcagcataaacaatcaattgattcattcttcttgataccaagttgtggaaactctaggattgtttgctgtatatctccagaattgctgttttgattgaatttaggtcgcttagctcagttgatagagcaccacattttttgtggtagaaatcgatttgtttgacagtctctaaccatgggtaaagagaagtttcacatcaacattgtggtcattggccacgtcgattctggaaagtcgaccaccactggacacttgatctacaagttgggtggtattgacaagcgtgtcattgagaggttcgagaaggaggctgctgagatgaacaagaggtccttcaagtacgcatgggttttggacaaacttaaggctgagcgtgagcgtggtatcaccattgacattgctctctggaagttcgagaccaccaagtactactgcactgtcattgatgctcctggccatcgtgatttcatcaagaacatgatcactggtacctcccaggctgattgtgctgtccttatcattgactccaccactggtggttttgaggctggtatctccaaggatggtcagacccgtgagcacgctctacttgctttcacccttggtgtcaagcagatgatctgctgttgtaacaagatggatgccactacccccaagtactccaaggccaggtacgatgaaatcatcaaggaggtgtcttcctacttgaagaaggttggttacaaccccgacaaaatcccatttgtgcccatctctggatttgagggtgacaacatgattgagaggtccaccaaccttgactggtacaagggaccaactctccttgaggctcttgaccagatcaacgagcccaagaggccgtcagacaagccccttcgtctcccacttcaggatgtctacaagattggtggtattggaacggtgccagtgggacgtgttgagactggtatgatcaagcctggtatggttgtgacctttgctcccacaggattgaccactgaggtcaagtctgttgagatgcaccacgagtctcttcttgaggcacttccaggtgacaacgttgggttcaatgttaagaatgttgctgtcaaggatcttaagagagggtacgtcgcatccaactccaaggatgaccctgccaagggtgctgctaacttcacctcccaggtcatcatcatgaaccaccctggtcagattggtaacggttacgccccagtcctggattgccacacctctcacattgcagtcaagttctctgagatcttgaccaagattgacaggcgttctggtaaggagattgagaaggagcctaaattcttgaagaatggtgatgctggtatggtgaagatgactccaaccaagcccatggttgtggagaccttctctgagtacccaccacttggacgtttcgctgtgagggacatgaggcagactgttgcagtcggtgttatcaagagtgttgacaagaaggacccaactggagccaaggttaccaaggctgcagtcaagaagggtgccaagtgaactctgaacatcaaaactctttccgctgatgaaatgaaggactattttagtttctttactttagtagtttggtatttggttggttgtttgtgttaccgcttcgtttattctccgtcggagctcaattctcggaattgggttcttgatcggaggtggcggaggtacttggcactcgagcttcctgtttatttcttttgttttgtgttatgattttgaacctttttggatattcggttatatgatatttgtgtcattcctgcggatattatcttatcttatcaacttaagtctgtttttcttctctaaaattcaacgatcagcctctgaagtttcttacatggtaaaattgtaatctagaactttaaacaacattggagaaacaagtctatatcagttt

SEQ ID NO: 208 - DNA, 1408 bp, Arabidopsis thaliana
  source 1..1408: mol_type = other DNA
tcatgatccttaatcttagctcctagggtttctgtgaattagtctaattttacaattttatgcctttgaggatatatcttaatgtgaaagttttaaacttgtaactcaaaaaccatttgttagttagtaatcatcatcattctgaattgagctttgtttgaatttgagtagggcaacgaaagattcaagttagttgcggtggtaagagtgaggtgttgaagagtgatacaatggaacctcatgaagcagaaacgtttgtgaacaagagaaccctttatgctcccattgagccatatagcagtggaaatttaaaggtttcagatgttcacactctctactgggagcaatcaggcaaacccgatggacacccggttgtttttcttcacggcgggccgggaggagggacggctcctagtaataggagattcttcgatccggagttttatcggattgttctgtttgatcagagaggtgcggggaagagtacaccgcatgcttgtttggaggaaaacaccacatgggaccttgtgaatgatattgaaaaactaagagaacatttgaaaatacctgaatggctggtttttggaggatcatggggaagcactctagctcttgcatacagtcagtctcaccctgacaaggttactggtttggtccttagagggatctttttgttgcgtaaaaaggagattgactggttttacgagggcggtgctgctgctatttaccctgatgcttgggaggagttcagagatcttattccagaaaacgagagaggtagtagccttgtggacgcgtatcacaaaagattgaattctgatgatctggaaattcaatacgcagctgcaagagcttggactaaatgggaaatgatgactgcttatcttcgtccaaatcttgaaaacgttcaaaaggcagaggatgataaattttcattggcgtttgcaaggattgaaaatcactactttgttaacaaaggtttcttcccgtcggactcacacttgctagacaatgttgataaaatacgacatatcaagaccaccattgttcagggaaggtatgacgtatgctgtcctatgatgtctgcttgggatctgcacaaagcatggccagaggcagaactcaagattgtttatgatgctgggcactcggcaaatgagccaggaatatcagcggaactcgtggttgccaatgaaaagatgaaagcacttatgggatgaagtatttgatggaatgaaagctgctcattttgttcatttctttgggacctttgttcatattttcaagtttgttgctgcaaagtaaatatcacattcagagttagttgttaacgcgaaaccgaatgttatttccatggacgtttatacccaaatgaaaaagacaatgacataggtgcagtgttttgaaa

SEQ ID NO: 209 - DNA, 1213 bp, Arabidopsis thaliana
  source 1..1213: mol_type = other DNA
acagagttcagaatatcgacggcgacaatttctctctccggctaacttttttctagagaaagctatggcggacgaagaagatgagttactggagacagaactaagttacgaccagaaaaaggaaatcgccaagtggtttttcctcaacgctcccgctggagaaatcaattacgtcgctaaagatttaaaggcggttttgagcgatgaggaagtgtacaatgaagcagccatggaagcatttccagtgtacaacaaaactcacatgatttgccttgaaatgcccagtggagctggtgatgtgattgtctcatcttacagtgaaattaatgaaaatgagtaccttgatccaagaactgcccaggttgccatagttgatcatgttaaacagatttgtacaaaggtacggcctgcaaatgatgaagaacttccttctttgtatattgaggagtacagatatgctcttgatgctgaaatccagagatatgttagtgaatcttatccgaaaggtatgtctgcagttaattgtgtgaaagggaaagataatgagggtccagggtccgactttgagcttgttgttataattactgctatgagacttagtccccagaacttctgtaatggaagttggagatctgtatggaacatagatttccaggatgaatcccaggtgcttgacataaaagggaaattgcaggtaggagctcattattttgaagagggaaatgtggaattagatgcaaaaaaagatttccaagattcaacaatatttcagtctgcagatgactgcgcgattgccatagccaatattatcaggcaccatgaaacagagtaccttgcatctctagaggtagcttattcaaaattaccggataacactttcaaggatttgaggaggaaacttccggtgacacgaactctgttcccatggcagaacactttacagttcagcctaacaagagaggtcgagaaagaacttggacttggcaagataagtattatttctgttgtgtcttgtctttcctttggagtaatgtatacaaatcgcattagtaagtatgatagatattgttccgcaagccgcatgaaagaattgaagtttggtcccaaaaagagctccgttgccgggaatcgaacccgggtctcctgggtgaaagccagatatcctaaccgctggacgacaacggatatgttattggaactgttaaagaactaa

SEQ ID NO: 210 - DNA, 2160 bp, Arabidopsis thaliana
  source 1..2160: mol_type = other DNA
ctcgtctccttctccacttggatctcaaaagtccgatccaaaaccaaatccgccataactctctctctctctcccacttcccacagagagacacattcatgccagcaatgccaggtctcgtctcagtcaaaactccttccgatacaccgccgctacgtgtagccgtaccagacacgcaacctctctcaaacccgccgcgaactccgatgaagaaaaccccttcctctacaccatctcgatccaaaccatcaccaaatagatccaccggcaaaaaagattccccaaccgtatcctcctccaccgcagccgtaatcgacgtcgacgatccatctctagataatccagatctcggtcctttccttctaaaattagctcgtgacgctattgcttctggtgaaggtcctaacaaagcattagattacgcgatccgagcaactaagtcctttgagagatgttgcgccgccgttgctccgcctattcccggtggtagcgatggtggacctgttcttgatctcgctatgagccttcatgttctcgctgctatttactgtagtctcggccgatttgatgaagctgtaccacctcttgaacgagctattcaagtccctgatccgactcgtggccctgaccactctctcgctgcgttttctggtcatatgcaacttggtgatacgttgtctatgcttggtcagattgatagatctattgcttgttatgaagaaggtcttaagatccagatccagactttgggtgatactgatccacgagttggtgaaacttgcaggtacttagcagaagcatatgttcaagcaatgcagttcaataaagcggaagagttatgcaagaagactctagagattcaccgagcgcatagtgaacctgcttcactggaggaagcagctgataggagattgatggcaattatctgcgaggctaaaggagattatgaaaacgctcttgagcatcttgttcttgctagtatggctatgattgcgagtggacaagaatctgaggttgcatcgattgatgttagtattgggaatatttacatgtcgctgtgccggtttgatgaagctgtgttttcatatcagaaggctcttactgtgtttaaggcttctaaaggagagactcatccgacggttgcgtcagtgtttgttagattggctgagctttaccatagaacagggaaacttcgtgagtctaagtcttactgtgagaatgcgttgaggatatacaataagccggtgcctgggacaactgtggaggagattgctggtggattaactgagatttctgctatttatgagtctgtagatgaacctgaggaagcattgaagctacttcagaagtctatgaaattgcttgaggataagccaggacagcagagcgccattgcagggttagaggcacggatgggagttatgtattacacagtggggaggtacgaggacgcaagaaacgcgtttgagagcgctgtgacaaagctacgggcagcaggtgagaaatctgccttcttcggtgttgtattaaaccagatgggactagcctgcgttcagcttttcaagattgatgaagcgggtgagctgtttgaagaagcaagaggtattcttgaacaagaacgtggaccgtgcgatcaggacactctcggtgtttatagcaatctagctgctacctatgacgctatgggaaggatagaggatgcgattgagatattagagcaggttctgaagctgagagaggaaaagctcgggactgcgaatccggattttgaagacgagaagaaacgtttggccgagcttttgaaggaagctgggcggtcgcggaactacaaagccaagtcgctccagaatctgattgatccaaatgcaagacctccgaagaaagagtcttctgctaagaaatggcccagcctcggtttcaagttctgaggaaaaaattatcatataaaataaatcatccacatatatattcaatattttattttattgttattgttatattttttatctgatctattttctgtatcataatctgaggttgatgaattaatttcatgtatagtgtttttatttgagtttggtgaaatactgccaagatggtgagctgtttgtttgagtttgaaatttctttaattcaatcacttttttcttcttctgataa

SEQ ID NO: 211 - DNA, 2248 bp, Arabidopsis thaliana
  source 1..2248: mol_type = other DNA
ctcgtctccttctccacttggatctcaaaagtccgatccaaaaccaaatccgccataactctctctctctctcccacttcccacagagagacacattcatgccagcaatgccaggtctcgtctcagtcaaaactccttccgatacaccgccgctacgtgtagccgtaccagacacgcaacctctctcaaacccgccgcgaactccgatgaagaaaaccccttcctctacaccatctcgatccaaaccatcaccaaatagatccaccggcaaaaaagattccccaaccgtatcctcctccaccgcagccgtaatcgacgtcgacgatccatctctagataatccagatctcggtcctttccttctaaaattagctcgtgacgctattgcttctggtgaaggtcctaacaaagcattagattacgcgatccgagcaactaagtcctttgagagatgttgcgccgccgttgctccgcctattcccggtggtagcgatggtggacctgttcttgatctcgctatgagccttcatgttctcgctgctatttactgtagtctcggccgatttgatgaagctgtaccacctcttgaacgagctattcaagtccctgatccgactcgtggccctgaccactctctcgctgcgttttctggtcatatgcaacttggtgatacgttgtctatgcttggtcagattgatagatctattgcttgttatgaagaaggtcttaagatccagatccagactttgggtgatactgatccacgagttggtgaaacttgcaggtacttagcagaagcatatgttcaagcaatgcagttcaataaagcggaagagttatgcaagaagactctagagattcaccgagcgcatagtgaacctgcttcactggaggaagcagctgataggagattgatggcaattatctgcgaggctaaaggagattatgaaaacgctcttgagcatcttgttcttgctagtatggctatgattgcgagtggacaagaatctgaggttgcatcgattgatgttagtattgggaatatttacatgtcgctgtgccggtttgatgaagctgtgttttcatatcagaaggctcttactgtgtttaaggcttctaaaggagagactcatccgacggttgcgtcagtgtttgttagattggctgagctttaccatagaacagggaaacttcgtgagtctaagtcttactgtgagaatgcgttgaggatatacaataagccggtgcctgggacaactgtggaggagattgctggtggattaactgagatttctgctatttatgagtctgtagatgaacctgaggaagcattgaagctacttcagaagtctatgaaattgcttgaggataagccaggacagcagagcgccattgcagggttagaggcacggatgggagttatgtattacacagtggggaggtacgaggacgcaagaaacgcgtttgagagcgctgtgacaaagctacgggcagcaggtgagaaatctgccttcttcggtgttgtattaaaccagatgggactagcctgcgttcagcttttcaagattgatgaagcgggtgagctgtttgaagaagcaagaggtattcttgaacaagaacgtggaccgtgcgatcaggacactctcggtgtttatagcaatctagctgctacctatgacgctatgggaaggtaaagtcctaaagaaaactatctgactatgtaattagatcagacatccttaatgaaacattaaagtctttcttcgtgatggctatgtaatttgcaggatagaggatgcgattgagatattagagcaggttctgaagctgagagaggaaaagctcgggactgcgaatccggattttgaagacgagaagaaacgtttggccgagcttttgaaggaagctgggcggtcgcggaactacaaagccaagtcgctccagaatctgattgatccaaatgcaagacctccgaagaaagagtcttctgctaagaaatggcccagcctcggtttcaagttctgaggaaaaaattatcatataaaataaatcatccacatatatattcaatattttattttattgttattgttatattttttatctgatctattttctgtatcataatctgaggttgatgaattaatttcatgtatagtgtttttatttgagtttggtgaaatactgccaagatggtgagctgtttgtttgagtttgaaatttctttaattcaatcacttttttcttc

SEQ ID NO: 212 - DNA, 1337 bp, Arabidopsis thaliana
  source 1..1337: mol_type = other DNA
ttaaagtaatgacttttttttatatgatatattctcaattttgttactcggaaacaatagaaaggataacgaatcgtttctcttcgatcgaaagaatatatatttggaagttttctctgcttttttgttttcgtttgatagaatgggaacagttctcgattcgcattttctggctctcactgctattgtcactgtgatttaccagttcatattcttcgtaatcacggctcttttcaaatttgatcaagtcactgactttgctggtagcacaaacttcgttatacttgctgtgttaacacttgttctcaaagcctcttggcattttcgacagatagtattgactttgctagttgtggtatggggtcttcgcttggggattttccttctaatgaggatcttgcaatggggggaagatcgtcgctttgatgaacagcgtggaaatatagtgagactaatcattttctggactcttcaggctgtgtgggtttggacggttagcttacctttaacacttgttaatgcaagtgatggtggtggatctcttaaacccgcagatgttatcggttggactatgtgggttttcggtttcttgattgaagctgcagctgatcaacagaagctatcattcaaaaactctcctgaaaacagaggaaaatggtgtgatgttggagtctggaagtattcaagacatccaaactacttcggtgagatgttactgtggtggggaatctttgtggctgcatcgcctgtgcttgaaggtgcagagtatcttgtcatattcggaccactctttctcactttgctacttctattcgtcagcggcataccattactcgaggcatcggctgacaaaaaacatggaaactcaggagcttacagatcctacaagaagacaacaagtcctctgattctgttcccaagaggagtgtatgggaacttaccaggatggttcaagacggtctttctcttcgagtttccattttacagccgaaatctccctcaagagggtagactccaaaacagcaaagagaaagaagtaaaagatgactgatttgtgatactcatatttgagttattgttcttcacaacactctttgataaaatttgtgtaaaaaattgcccgtaagaaactgaatttgtttgaaatattggacagtcttgacagattatctcagttgtatttggttcttttttttatgtgattttgttttttggttaaaaaaagttcccattccaagaatcgaacccgggtctcctgggtgaaagccaaatatcctaaccgctggacgacatcggatttgattatttgattatttaattactttatatgttgattt

SEQ ID NO: 213 - DNA, 1079 bp, Arabidopsis thaliana
  source 1..1079: mol_type = other DNA
gaagagtaagagtaaaaaacaaaaccttcgtctctaacaaatgtgtggcagctgattgaaactagtagtggtgaaggagaaggagaatgggagatctctacgctttggacttcgatggagttctctgtgatagttgtggagaaagctctctatctgctgttaaggcagccaaagtgagatggcctgatttgttcgaaggagttgattccgctttagaggaatggatcgtcgatcaaatgcatattgtgagacctgtggttgaaactggctatgaaaatcttctacttgtgcggctgttattggagaccaagataccgtctattcgaaaatcttcagttgcagaggggttgacggttgatgggatactggagagctgggcaaagtttaagcccgtgatcatggaagcttgggatgaagatagagatgctttggtagatttgtttggaaaagttagagatgattggattaacaaggacttgacaacttggattggtgccaatagattctacccaggagtttcagatgcactgaaatttgccagctcaaagatttatatcgttaccacgaaacagggccgctttgcagaagcgttactgcgtgaaatagcaggtgtaatcataccatcagaaaggatttatggtttgggttcagggccaaaggtggaagttctaaagctacttcaagataaaccagaacatcagggcttgacgctgcatttcgtggaagacagactcgcaacgctgaaaaatgtcataaaagagcctgaattagacaaatggagtttatacctaggaacctggggatacaacactgagaaagagagagctgaagcagcaggcattcccaggattcaggttattgagctctccacgtttagcaataagcttaaatgagtgcatatactcattggaaaagaaacaaagctttcctctttattgtattctacggcattcaacggaaactaaaaaccaaacatgtttaggtaagattcagtaaaagagttgaactttcagtactacccattatagaagcttttctgtgacctataattcatccttagtaagatcattcttgttacc

SEQ ID NO: 214 - DNA, 1064 bp, Arabidopsis thaliana
  source 1..1064: mol_type = other DNA
gaagagtaagagtaaaaaacaaaaccttcgtctctaacaaatgtgtggcagctgattgaaactagtagtggtgaaggagaaggagaatgggagatctctacgctttggacttcgatggagttctctgtgatagttgtggagaaagctctctatctgctgttaaggcagccaaagtgagatggcctgatttgttcgaaggagttgattccgctttagaggaatggatcgtcgatcaaatgcatattgtgagacctgtggttgaaactggctatgaaaatcttctacttgtgcggctgttattggagaccaagataccgtctattcgaaaatcttcagttgcagaggggttgacggttgatgggatactggagagctgggcaaagtttaagcccgtgatcatggaagcttgggatgaagatagagatgctttggtagatttgtttggaaaagttagagatgattggattaacaaggacttgacaacttggattggtgccaatagattctacccaggagtttcagatgcactgaaatttgccagctcaaagatttatatcgttaccacgaaacagggccgctttgcagaagcgttactgcgtgaaatagcaggtgtaatcataccatcagaaaggatttatggtttgggttcagggccaaaggtggaagttctaaagctacttcaagataaaccagaacatcagggcttgacgctgcaactcgcaacgctgaaaaatgtcataaaagagcctgaattagacaaatggagtttatacctaggaacctggggatacaacactgagaaagagagagctgaagcagcaggcattcccaggattcaggttattgagctctccacgtttagcaataagcttaaatgagtgcatatactcattggaaaagaaacaaagctttcctctttattgtattctacggcattcaacggaaactaaaaaccaaacatgtttaggtaagattcagtaaaagagttgaactttcagtactacccattatagaagcttttctgtgacctataattcatccttagtaagatcattcttgttacc

SEQ ID NO: 215 - DNA, 1133 bp, Arabidopsis thaliana
  source 1..1133: mol_type = other DNA
gcagctgattgaaactagtagtggtgaaggagaaggagaatgggagatctctacgctttggacttcgatggagttctctgtgatagttgtggagaaagctctctatctgctgttaaggcagccaaagtgagatggcctgatttgttcgaaggagttgattccgctttagaggaatggatcgtcgatcaaatgcatattgtgagacctgtggttgaaactggctatgaaaatcttctacttgtgcggctgttattggagaccaagataccgtctattcgaaaatcttcagttgcagaggggttgacggttgatgggatactggagagctgggcaaagtttaagcccgtgatcatggaagcttgggatgaagatagagatgctttggtagatttgtttggaaaagttagagatgattggattaacaaggacttgacaacttggattggtgccaatagattctacccaggagtttcagatgcactgaaatttgccagctcaaagatttatatcgttaccacgaaacagggccgctttgcagaagcgttactgcgtgaaatagcaggtgtaatcataccatcagaaaggatttatggtttgggttcagggccaaaggtggaagttctaaagctacttcaagataaaccagaacatcagggcttgacgctgcagtaactcacgttctatatatctatcaataattcttctatctgcatgacttggtaatggtcaccattagtttacaatccctatgcatttttaatttggccagtttcgtggaagacagactcgcaacgctgaaaaatgtcataaaagagcctgaattagacaaatggagtttatacctaggaacctggggatacaacactgagaaagagagagctgaagcagcaggcattcccaggattcaggttattgagctctccacgtttagcaataagcttaaatgagtgcatatactcattggaaaagaaacaaagctttcctctttattgtattctacggcattcaacggaaactaaaaaccaaacatgtttaggtaagattcagtaaaagagttgaactttcagtactacccattatagaagcttttctgtgacctataattcatccttagtaagatcattcttgttacc

SEQ ID NO: 216 - DNA, 700 bp, Arabidopsis thaliana
  source 1..700: mol_type = other DNA
acatcttctcttttcttccccagcaaaaaatgagtctgcaaaatagaaaattcaagctgcgattgcgaggaagagaagagacttcagatctagcttgatccggtgataatggcgctcgagtgggtggttttaggttacgcggcggcggctgaagcgattatggtggtgctattgacgatgccaggactcgacggactccgcaagggattagtcgccgtcacgcgtaatctcctcaaaccgtttctctccatagttcctttctgtctcttcctcttaatggacatctactggaaatacgaaactatgccttcgtgcgatggcgattcctgcacaccgtcagagcatcttcgtcaccagaagtccatgatgaaatcccagcgaaacgcgcttctcattgcggctgcacttgtgttctactggatcctttactcagtcaccaatttggttgtcaagatcgagcatcttaaccagaggatcgagaggctcaggaacaaggattgatcgtctcgtctcaggttcaattagtgtaatgccttttttggttatctttcccatttggctaggttttctctttttcaacgtttctgatatgtgtcttttatcttgtggagattgaaggattttatcctacttttctatgtttggattcttatttgtaaccagattgtgattgtttcttgttgaattttgattgactcta

SEQ ID NO: 217 - DNA, 1754 bp, Arabidopsis thaliana
  source 1..1754: mol_type = other DNA
tctccacaaacaaacttgtactagaaaaatccctaaatctcaacaaaaccctaatttcaaggaagacgaagaaagaatgagttacagagtcgctaaagcatcgcagtatcttgcgatcaccggtggtgggatcacagacatcaagcttgcaaagaagtcttgggtgtttccatggcagtcttgcaccgtcttcgacgtttctccggtgaactacacgttcgaggttcaagctatgagttccgagaaacttccctttgttattcctgctgttttcacaatcggtcctcgcgttgatgatcctcacgctctcttgttgtatgccatgcttatgtctcaacacgataagcactcgaatcacgtcaatgagcttgttcagggtgttatcgaaggagagactcgtgttcttgttgcttccatgactatggaagaggtcttcaaaggaacaaaggagtttaagaaggaagtgtttgacaaggttcagctagagctaaaccagtttggtcttgtgatctacaacgccaatgtgaagcagcttgttgatgtgcctggacatgagtacttctcttacttgggccagaagactcagatggaagcagcaaaccaagccaagatcgatgtagcagaggcaaagatgaagggagaggttggtgccaaagaaaggactggactcacaatccagaatgcagccaagattgacgctgagtcaaagatcatctctactcaaaggctaggagaagggacaaaagaggagatcaaggtgaagactgaagtcaaagtgtttcagaatgagaaagaggctcttgttgctaaggctgatgcagcacttgcgattcaaaaggctgctttgtcccaaaactctcgtgtggctgaggttgaagcagccaaggctgttgctttgagggaagctgagcttcagactaaagtagagaagatgaatgcattgactcggacagagaagcttaaagctgagttcctcagtaaagccagtgttgagtatgaaactaaggtgcaagaagcgaactgggagttgtacaataagcaaaagcaagcagaagctgttctttacgagaagcagaagcaagcggaagcgacgaaagctgcagctgatgctgccttctattcaaaacagaaagatgcagagggacttgtcgcaatggccgatgctcaggggacttacctcaagaccctcttaggcgcagttaacaatgattactcagccatgagagacttcttgatgatcaacaatggcatttatcaggatattgccaagaccaatgctgttgcgatcagggacctgcagcctaagatcagcgtgtggaaccatggtggtgctgagcaggggatgaacggtggtggtaaagctacgatgaacgatattgctggactctacaagatgttaccaccggttcttgatacggtttatgagcagacagggatgcagccaccagcttggattggtacattgcgtggtgctgagcccaaacaatcacttcatgctcaacaacacagaggttgagatctcaaatgttgcttctttaattatacttttagctgttggggtttgttttctttttcttatgttatagaacttggaagttagagtttgaagttaaaacaatagtatctgacaaatgcgacttcgactagtgatttcgataaggatgtgtttttcctttttcaaatgtcagatttattattgcgtgttgaagatacctttcttctctcttgatacactaatataaataacatgttta

SEQ ID NO: 218 - DNA, 683 bp, Arabidopsis thaliana
  source 1..683: mol_type = other DNA
cacagaaccctccctctctagctctaaaaacatcccacaaactatctttcaacaacacaagacatatcacgaaacaaatcaaagcaaagatcatgaacacgattccagccgaactcactggatacttccactatctaccaccagataaatataataaccaaacaccaatcatggagtctgagtacttcaacatgccctcttctcctacttcttgttcctccttctaccacctcaatggtctgatcaacaacaacaactactcttcatcatccaatggtcaggatctaatgacgagcaacaactcaacttcagacgaagatcaccaacaaagtatggtcatcgatgagaggaaacaaaggagaatgatctctaacagggaatctgctcgtaggtcaagaatgagaaagcagagacatctcgatgagctttggtctcaagtgataagacttcgcactgataaccactgtcttatggataagctgaaccgcgtatctgaaagccatgagcttgctttaaaggagaacgctaagcttaaagaggaaacttctgatctcagacagctgatctctgagataaagtcacacaacgaagacgacaacagttttttaagagagctcgaagattcgatatcaaactctagatcggattcgaaccaaagcggcagagattttgagttgtgttaa

SEQ ID NO: 219 - DNA, 432 bp, Arabidopsis thaliana
  source 1..432: mol_type = other DNA
taagctagaggtttttagttctctgtgtggagtacggctgcattgtttctaccagtaatttgtgggatttccaagctccagtttttcgaatatggcggaagtaagtcttggtatgcttatagacattgtggacgaagagtggatgagggacactttgcctgatgatgatcttccattgcctccagtgcttgctgttaaaactgatgatactgaagaaaccaatcaggaaactcagcaagcagatgctgaaacttggcgtgatctagctcttgatacacagtaatagcctcttaagtacaatggaaaattcttacaagtcttgtttctcgttgtgtatgatcaaaaccaaaactgttcctggctttgtatttttttttggtagaagaggggggcgaacccccatttttattaaaactgaaaaactaaaaaaaa

SEQ ID NO: 220 - DNA, 3538 bp, Arabidopsis thaliana
  source 1..3538: mol_type = other DNA
gaagtagaagaagagaagtatctggagagagagaggcgtgcgatttccgcatcgaagaaacagtacggagttttatccgctccgattctgtggaagataatggggaaaggaggtgaagattgcgggaataagcaaactaatagttctgaattggtcaagtctgatacttttcctgcttggggtaaagatgtgtcggaatgcgaagagaagtttggtgttagtcgtgagaaaggtttgagcactgatgaggtgttgaaacggcatcagatctatggtttgaatgaattagagaagcctgagggtacttctatttttaagctgattttggaacagtttaatgatacattggttcgtattcttttggcggctgctgttatttcctttgtcttagccttctttgatggcgacgaaggtggtgagatgggtatcaccgcatttgttgagccgcttgttatttttctcatcttgattgttaatgccattgttggcatctggcaagaaactaatgctgagaaggcgttggaagctttgaaagagattcagtctcagcaagctaccgtcatgcgtgatgggactaaggtttccagtttgcctgcaaaggagcttgttcctggtgatattgtggaactgagggttggtgataaggtgcctgctgatatgcgtgtggttgctttgatcagttcaactttgagagtcgagcaggggtccttaacaggagagagtgaggcagtgagcaaaaccaccaagcacgtggatgagaatgctgatattcaaggaaagaaatgcatggtgtttgcaggaaccactgtcgtgaatgggaactgcatatgtttagttacagatactgggatgaatactgagattggaagggttcactcacagatccaggaagctgcacaacacgaggaagatacccctctgaagaaaaaacttaacgagtttggggaagttctgactatgatcattgggttaatttgtgcgttagtgtggctcatcaatgtcaaatactttctgtcatgggagtatgttgatggctggcccagaaactttaagttctccttcgagaagtgcacatattactttgagattgctgttgcactggcggttgctgcaattccagaaggtctgccagcagttattaccacatgtttggctcttggcactaggaagatggctcagaagaatgctctggttaggaagctacccagtgtggagactcttggatgcaccaccgtgatatgctctgacaaaactggaactcttacaaccaatcagatggctgtttcaaagctcgttgctatgggttcgaggatcggaactcttcgatctttcaatgttgaggggacctcgtttgatcctcgagatggaaagattgaagattggccaacgggtcggatggatgcaaatcttcagatgattgcaaaaattgctgccatttgcaacgatgctaatgttgagaaatctgatcagcaatttgtgtctaggggcatgcctactgaggcagctttgaaggttttggttgagaaaatgggatttcccgaaggattgaatgaagcttcatccgatggcaatgtcttacgttgttgtcggctatggagtgaactagaacaacggattgccacccttgagtttgaccgtgaccggaaatcaatgggagttatggtggattccagctcaggaaagaaactgctactggtcaagggtgccgtagaaaatgtattggaaaggagtacccatattcagctacttgatggttccactcgagagcttgaccaatactcaagggatcttattttacaaagcctacatgatatgtccttgagtgcattaagatgtctcggatttgcatactcagatgttccatcagattttgccacttatgatggcagtgaagaccatcctgctcatcagcaacttctcaacccatccaattattcatcaattgagtccaatctagtatttgttggatttgttggtttaagggatcctccaaggaaagaggtgcgtcaagccattgcagactgcagaacagcaggtattcgggtcatggttatcaccggagacaacaagagcacagcggaagcaatctgccgtgaaattggcgtatttgaagcagatgaagatatctcctcgagaagcttgacaggaaaagaatttatggatgttaaagatcagaagaatcatctaagacagacaggagggctcctgttctcaagggctgaaccaaaacacaaacaagagattgtcaggttactgaaagaagatggagaagtggttgccatgactggagatggagtcaatgatgctcctgccctgaagttggctgatattggtgtggctatgggcatttccgggacagaggtagcaaaggaggcatctgacttggtgttggcagatgataatttcagcaccattgttgcagctgttggtgaaggcaggtccatctataacaacatgaaggccttcatcaggtacatgatctcttccaacatcggtgaggttgcatcgatattcttgacagctgctcttggaatcccagaaggcatgatacccgttcaacttctgtgggtgaatctcgtaacagatggtcctccagctactgctttgggattcaacccccctgacaaggatattatgaaaaagcctcctcgtaggagcgatgactctcttatcactgcgtggatcctcttccgctatatggtgattggtttgtatgtgggagtagcaacggtgggagtattcatcatatggtacacacacaacagctttatgggcatagacctgagccaagacggtcacagccttgtgagctattcacagctcgcacattggggccaatgctcttcatgggaaggcttcaaagtgtctcctttcacagcaggctctcagactttctcgttcgactcaaacccttgcgactattttcagcagggtaaaatcaaggcatccacactctccctctcggtgttggtggccattgaaatgttcaactctctgaatgcactctcagaagatggcagcctggtgacgatgccaccctgggttaacccgtggctgctcctggcaatggctgtctcatttggactccactttgtgatattgtacgtcccattcttggctcaggtgtttggtattgtgccactgagcttgaacgagtggctgctagtgctggctgtgtcgctgccggtaattttgatagacgaagtgctcaagtttgtaggaaggtgcacgagtggctaccgctactcacctcgcactccctcagctaagcaaaaggaggagtaagaaaataattcaaaaggcattattattagactcttagttttagctgaaattaactttgttgctttactcatccttgctatatttaattctccaaacgataatttttggtttgaaaaagaaaagtttctaactttggttcatggacacagcccgtgatagaaagatgaatgtttcaaccaaaccttgattctttatgtaaacttttatggtcgaccaaattggtttttaagactgatggtaatttattttgggc

SEQ ID NO: 221 - DNA, 2087 bp, Arabidopsis thaliana
  source 1..2087: mol_type = other DNA
ataaacctacttcctttccttgcttcagctccccttttcttatccggaaaccaaaaacttccggtgataaaacctccttcgtcatctctctacttcttgctgaattttgtctgtctttgaagtggataaaatggcgtcaactttcactgccacatcttctattggttcaatggttgctccgaatggtcacaaatctgataagaaacttatcagcaagttgtcttcaagctcatttggaaggagacaaagtgtgtgtcctagacccagaagatccagttctgcaattgtgtgcgcagcaaaggaattacatttcaacaaagatgggactaccattaggagacttcaagctggtgtcaacaagctcgcagatcttgttggtgttacacttggacccaaaggacgaaatgttgttcttgagagcaagtatggatcaccacgaattgttaatgatggtgtgactgttgcaagagaggttgagttggaagacccggttgagaacattggcgctaagcttgtgaggcaagcggctgccaaaaccaatgacttggctggtgacggtacaacaacatctgttgttcttgcacaaggttttatcgctgagggtgtcaaggtggtagctgctggtgcaaaccctgttttgatcaccagaggtattgagaagacagcaaaggctttggttaccgagctcaagaaaatgtctaaggaggttgaagacagtgaacttgcagatgttgcagctgttagtgcgggtaacaacgacgaaattggaaatatgattgctgaagcaatgagcaaagtgggcaggaaaggtgtggtgacccttgaggagggtaaaagtgccgagaacaacctctacgtcgtagaaggaatgcaatttgaccgtggttatatctctccttactttgtgacagacagcgagaaaatgtccgtcgagttcgacaattgcaagttacttctcgttgacaagaaaattaccaatgcaagggatcttgttggtgttttggaggatgcaattagaggcggatacccaatattaattattgctgaagacattgaacaagaagctttagcgactcttgttgtaaacaagcttagaggtacactgaagatagcagctctcagagctccagggtttggagagcgcaagagccaataccttgatgatattgccattctaactggagcaactgtgatccgtgaggaagttggtctttcacttgacaaagctggaaaagaggttcttggtaatgcttcaaaggttgtcctcacaaaggagacctcgaccatagtgggtgatggaagcacacaggatgcagtgaaaaagcgtgttacacagattaaaaatcttattgagcaagcggagcaagattacgagaaggagaaactcaatgagagaattgcaaagctctctggtggagttgctgtgattcaggttggagcacaaactgagacagaactcaaagagaagaagttgagagttgaagatgctcttaatgcaacaaaggccgctgtcgaggaaggtattgttgttggtggtggttgcactctgcttcgtcttgcttccaaggttgatgccattaaagccacgcttgataatgatgaagaaaaggttggagcggatatagttaaaagagcactaagttaccccctaaaactaatcgccaagaatgctggtgttaatggaagtgtagttagcgagaaggtgctttccaacgataacgtgaaattcggttacaatgctgcaaccggcaagtacgaggatttgatggctgcaggaatcatcgatccaacaaaggttgtgagatgttgcttggaacacgcagcttcggttgcaaagactttcttgatgtcggactgcgttgttgttgagatcaaggaacctgaaccagttcccgtcggcaacccaatggacaactcaggatatggatactaagaggagataggctaatattgaaacggattactttagcttgcgagtgtaatgattttgaacaagagaatgtgtaatgtgaaactgaaattttgaattaatcatcatatgaataaatcatgattccctcaatgcttcgaaatatctcgaaatcgcc

SEQ ID NO: 222 - DNA, 2093 bp, Arabidopsis thaliana
  source 1..2093: mol_type = other DNA
ttaaaagcaccttccaagcgagagtaaggagataaacctacttcctttccttgcttcagctccccttttcttatccggaaaccaaaaacttccggtgataaaacctccttcgtcatctctctacttcttggataaaatggcgtcaactttcactgccacatcttctattggttcaatggttgctccgaatggtcacaaatctgataagaaacttatcagcaagttgtcttcaagctcatttggaaggagacaaagtgtgtgtcctagacccagaagatccagttctgcaattgtgtgcgcagcaaaggaattacatttcaacaaagatgggactaccattaggagacttcaagctggtgtcaacaagctcgcagatcttgttggtgttacacttggacccaaaggacgaaatgttgttcttgagagcaagtatggatcaccacgaattgttaatgatggtgtgactgttgcaagagaggttgagttggaagacccggttgagaacattggcgctaagcttgtgaggcaagcggctgccaaaaccaatgacttggctggtgacggtacaacaacatctgttgttcttgcacaaggttttatcgctgagggtgtcaaggtggtagctgctggtgcaaaccctgttttgatcaccagaggtattgagaagacagcaaaggctttggttaccgagctcaagaaaatgtctaaggaggttgaagacagtgaacttgcagatgttgcagctgttagtgcgggtaacaacgacgaaattggaaatatgattgctgaagcaatgagcaaagtgggcaggaaaggtgtggtgacccttgaggagggtaaaagtgccgagaacaacctctacgtcgtagaaggaatgcaatttgaccgtggttatatctctccttactttgtgacagacagcgagaaaatgtccgtcgagttcgacaattgcaagttacttctcgttgacaagaaaattaccaatgcaagggatcttgttggtgttttggaggatgcaattagaggcggatacccaatattaattattgctgaagacattgaacaagaagctttagcgactcttgttgtaaacaagcttagaggtacactgaagatagcagctctcagagctccagggtttggagagcgcaagagccaataccttgatgatattgccattctaactggagcaactgtgatccgtgaggaagttggtctttcacttgacaaagctggaaaagaggttcttggtaatgcttcaaaggttgtcctcacaaaggagacctcgaccatagtgggtgatggaagcacacaggatgcagtgaaaaagcgtgttacacagattaaaaatcttattgagcaagcggagcaagattacgagaaggagaaactcaatgagagaattgcaaagctctctggtggagttgctgtgattcaggttggagcacaaactgagacagaactcaaagagaagaagttgagagttgaagatgctcttaatgcaacaaaggccgctgtcgaggaaggtattgttgttggtggtggttgcactctgcttcgtcttgcttccaaggttgatgccattaaagccacgcttgataatgatgaagaaaaggttggagcggatatagttaaaagagcactaagttaccccctaaaactaatcgccaagaatgctggtgttaatggaagtgtagttagcgagaaggtgctttccaacgataacgtgaaattcggttacaatgctgcaaccggcaagtacgaggatttgatggctgcaggaatcatcgatccaacaaaggttgtgagatgttgcttggaacacgcagcttcggttgcaaagactttcttgatgtcggactgcgttgttgttgagatcaaggaacctgaaccagttcccgtcggcaacccaatggacaactcaggatatggatactaagaggagataggctaatattgaaacggattactttagcttgcgagtgtaatgattttgaacaagagaatgtgtaatgtgaaactgaaattttgaattaatcatcatatgaataaatcatgattccctcaatgcttcgaaatatctcgaaatcgcc

SEQ ID NO: 223 - DNA, 1960 bp, Arabidopsis thaliana
  source 1..1960: mol_type = other DNA
gtgaacgaagataaatttcgctattcctctctctctctctctctctctctctctctctctctctcttcttcttcttctccgttcgtgctctggactcgctcgaaatcgatccgatgataatttatgcagctcgaaacttaatttagaatcatacctttttctggatttggctgacgaatccgttttcaattgcacgagttgcttcgccaggtttatcaacttaagaatgtctaattatcaaggggatgatgctgagtacatggaagatgtggatgatgaaatggatgaggatgatttagatgaggattttcgtggggatgatatggctgcctctgattccgatgtcgatgagtttgactactctaataataaaatagctgacacttcagctgaccaagcccggaaagggaaagacatccaggggattccttgggacaggcttagtattacgagagagacatacagacaaactagactagaacagtataaaaattatgaaaatgtccctaactctggggaatcttcagggaaagattgcatggccacacagaaaggggctcttttttatgatttctggcggaatacaagatctatcaaatcaagtattcttcatttccagttgaggaatttggtttgggcaacttctaagcacgatgtctaccttatgtcacaatatttggtgagccactattctactttgacatccggaaagcatgatgttctcaatgttcaaggtcatgtttccccatctgagaaacatcctggaagtttgttggagggatttacaaagactcaagtgagtacactcgcagtgagagataaatttttagttgccggtggatttcaaggagaacttatatgcaagcatcttgatagacctggtgtgagcttttgttcacgtactacttatgatgataatgctataaccaatgctattgagatttataacaaacccagtggggcacttcattttactgcgtcgaataatgattgtggagtcagagattttgatatggagagataccagcttgttaaccagtttcgctttccatggccggtgaatcacacatctttaagtcctgatggtaaattgttgacgattgtgggagacaaccctgaaagccttctggtagaccccaacactggaaagacactggcaacgctatcaggacatctggacttttcctttgcatcagcatggcacccagatggattcacgttctccacaggtaaccaggacaagacctgccgggtttgggacattcgtaacctgtcaaaatctgttgctgtcttgaggggtaaccttggagcaatccgatccattcgctacacatctgatgggaagtacatggccatggctgagccggctgactttgtccatgtgtatgatgtctcaaacgggtatgaaacggagcaagagatcgacttctttggggagatctcgggaatatcatttagccccgacacagaagcactcttcataggcgtttgggaccgcacttacggaagtctaattgagtatagccggcgccacaactactcctaccttgattcatttctatgatcacccgacataccagaggaataaaagagccgatacatatgtgctgctgcactctcctgtcgctctgttaaactaagaaacccgagaaaagagaaaacataaactcaagtcgatctctaatggtgtaaatgatgtgttgtgcaagtactagtaggaatgttgtcacagagagcggatttagttagttaatgttcatttcttgtatgagtgttggagttcgtgtattgtacataagtatatgaggttcgggtgagtttgcatcaagtctcaactcttggcaaagagacaactttactcgagtctgtggctcgttatttggctatggtcacttgtagaggaactcaatttgagatgtttctgccgattgttggatcaaattatatatcatgatgtttc

SEQ ID NO: 224 - DNA, 2741 bp, Arabidopsis thaliana
  source 1..2741: mol_type = other DNA
aatcggcgcgtgatcgagaaagatcttctttaacctctttgcaacaaatctactcaaaaccctaacccctttttcaaattacttccatctttagatcgaatcaaatcaatgtccgtcgacgatctccctccgtcgtcggcctccgccgtaacgccactaggctcctccgtgattccgattgtgaacaagctccaagacatattcgctcagctaggaagtcaatcgacgatcgctcttccgcaagtagctgtcgtcggaagtcaaagcagtggcaaatccagcgttctagaagctctcgtcggccgtgactttcttcctcgtggcaatgatatttgcacgcgccgacctctccgtctccagcttgttcagacgaaacctagctctgatggtggatccgatgaagagtggggcgagtttcttcaccacgatcctgttagacgtatctacgatttctctgagattcgccgtgagattgaggcggagactaatagagtatcaggagagaacaaaggtgtatcagatattccgattggtttgaagatattttcacctaatgttttggatattagtcttgtggatcttcctggtatcactaaggttcctgttggtgatcagccaagtgatatcgaagcgcgtataagaacgatgatcttgacttacattaaggaacctagttgcttgatacttgctgttagccctgctaatactgatttagcaaattcagatgcattacaaatcgcaggaaatgctgatcctgatggtcatagaaccataggtgtaatcacaaagttggatattatggacagaggtactgatgctcggaatcaccttctgggaaaaacaattcctcttcgacttggatacgtgggagttgtaaatcgtagtcaagaggatattttgatgaaccgtagcatcaaggatgctcttgttgcagaggaaaagttctttcgtagtcgtccagtttacagcggtctcacagatcgtttgggtgtccctcaactggcaaagaaattaaatcaggtccttgttcaacacatcaaggcactgcttcctagtttaaagtcacgtatcaacaatgcattgtttgctacagcaaaggagtatgagagctatggggatataacagagtctaggggtggtcaaggagctcttctcctcagttttattacaaaatactgcgaagcatactcctctaccctggaagggaaaagtaaagaaatgtcaacatctgagctctcgggtggagcaagaattctctacattttccaatcagtatttgttaagagcttagaggaggttgatccatgtgaagacttaacagctgatgatattcggactgcaatacaaaatgcaactggtcccagatctgcattatttgttccagatgttccatttgaagttcttgtcaggaggcaaatatctcgtttattagatcccagccttcagtgcgctaggttcatcttcgatgagctagtaaagattagtcatcagtgtatgatgaaagagttacagcggtttccagtcctgcaaaagcgcatggatgaggttattgggaactttctgcgagaaggtcttgaaccttcgcaggcaatgatccgagatcttattgaaatggagatggattatataaacacctcacacccaaattttatcgggggaactaaagccgtggagcaagcaatgcaaacagtcaaatcttctaggattccgcatcctgttgcgcggccaagggatactgtggagcctgagaggacggcttcttccgggagtcaaataaaaacccgatcttttctcggccgacaagctaatggaatcatcactgatcaggcagttccaactgcagcagatgctgaaagacctgcaccagctggaagcacaagctggagtggtttctcatcaatttttcgggggagtgatggtcaggcagctgctaaaaacaacctattaaacaaaccattcagcgaaactactcaagaagtgtatcagaacttgtcaacaatctatttaaaggagcctccaactattttgaagtcatctgaaactcattcagaacaggagtcagttgagattgagataacaaagctattattgaaatcctactatgacattgtaaggaagaatgtcgaggacttggtcccaaaagcaatcatgcattttctggtaaattacacgaaacgtgagctgcacaatgttttcatcgagaagctttacagggagaacttaattgaagaactgttgaaagagcccgatgagttagcgataaagaggaaacgcacgcaagagactcttcgcattcttcagcaggctaatcggacgttagacgagctgccgttagaagctgaatcggtcgagagaggttacaaaattggttcagaagcgaaacatgaggagttaccgggcacaagaagatcaaggacagaaactaatgggaacggacggcttcatatgtaaattcaaattgtgattgcacgcatgactcattcatttggtgatggaaaagatttgttttcttccctttaaatcggttgtgcgtggggaaagaaaggtgttcttaggtatagtgaaacgtgatgataaacagaggttttgttgtgagggtattatcaacctactggaaaatgtgacttaatatttgtttctcaaaaccgagaagaataatcaattcctaattttacttttcatagctttatttgttacgaaaaacaagaacgtacatgagacaatgtaatgataaatttatc

SEQ ID NO: 225 - DNA, 2749 bp, Arabidopsis thaliana
  source 1..2749: mol_type = other DNA
gagaaagatcttctttaacctctttgcaacaaatctactcaaaaccctaacccctttttcaaattacttccatctttagatcgaatcaaatcaatgtccgtcgacgatctccctccgtcgtcggcctccgccgtaacgccactaggctcctccgtgattccgattgtgaacaagctccaagacatattcgctcagctaggaagtcaatcgacgatcgctcttccgcaagtagctgtcgtcggaagtcaaagcagtggcaaatccagcgttctagaagctctcgtcggccgtgactttcttcctcgtggcaatgatatttgcacgcgccgacctctccgtctccagcttgttcagacgaaacctagctctgatggtggatccgatgaagagtggggcgagtttcttcaccacgatcctgttagacgtatctacgatttctctgagattcgccgtgagattgaggcggagactaatagagtatcaggagagaacaaaggtgtatcagatattccgattggtttgaagatattttcacctaatgttttggatattagtcttgtggatcttcctggtatcactaaggttcctgttggtgatcagccaagtgatatcgaagcgcgtataagaacgatgatcttgacttacattaaggaacctagttgcttgatacttgctgttagccctgctaatactgatttagcaaattcagatgcattacaaatcgcaggaaatgctgatcctgatggtcatagaaccataggtgtaatcacaaagttggatattatggacagaggtactgatgctcggaatcaccttctgggaaaaacaattcctcttcgacttggatacgtgggagttgtaaatcgtagtcaagaggatattttgatgaaccgtagcatcaaggatgctcttgttgcagaggaaaagttctttcgtagtcgtccagtttacagcggtctcacagatcgtttgggtgtccctcaactggcaaagaaattaaatcaggtccttgttcaacacatcaaggcactgcttcctagtttaaagtcacgtatcaacaatgcattgtttgctacagcaaaggagtatgagagctatggggatataacagagtctaggggtggtcaaggagctcttctcctcagttttattacaaaatactgcgaagcatactcctctaccctggaagggaaaagtaaagaaatgtcaacatctgagctctcgggtggagcaagaattctctacattttccaatcagtatttgttaagagcttagaggaggttgatccatgtgaagacttaacagctgatgatattcggactgcaatacaaaatgcaactggtcccagatctgcattatttgttccagatgttccatttgaagttcttgtcaggaggcaaatatctcgtttattagatcccagccttcagtgcgctaggttcatcttcgatgagctagtaaagattagtcatcagtgtatgatgaaagagttacagcggtttccagtcctgcaaaagcgcatggatgaggttattgggaactttctgcgagaaggtcttgaaccttcgcaggcaatgatccgagatcttattgaaatggagatggattatataaacacctcacacccaaattttatcgggggaactaaagccgtggagcaagcaatgcaaacagtcaaatcttctaggattccgcatcctgttgcgcggccaagggatactgtggagcctgagaggacggcttcttccgggagtcaaataaaaacccgatcttttctcggccgacaagctaatggaatcatcactgatcaggcatttccaactgcagcagatgctgaaagacctgcaccagctggaagcacaagctggagtggtttctcatcaatttttcgggggagtgatggtcaggcagctgctaaaaacaacctattaaacaaaccattcagcgaaactactcaagaagtgtatcagaacttgtcaacaatctatttaaaggagcctccaactattttgaagtcatctgaaactcattcagaacaggagtcagttgagattgagataacaaagctattattgaaatcctactatgacattgtaaggaagaatgtcgaggacttggtcccaaaagcaatcatgcattttctggtaaattacacgaaacgtgagctgcacaatgttttcatcgagaagctttacagggagaacttaattgaagaactgttgaaagagcccgatgagttagcgataaagaggaaacgcacgcaagagactcttcgcattcttcagcaggctaatcggacgttagacgagctgccgttagaagctgaatcggtcgagagaggttacaaaattggttcagaagcgaaacatgaggagttaccgggcacaagaagatcaaggacagaaactaatgggaacggacggcttcatatgtaaattcaaattgtgattgcacgcatgactcattcatttggtgatggaaaagatttgttttcttccctttaaatcggttgtgcgtggggaaagaaaggtgttcttaggtatagtgaaacgtgatgataaacagaggttttgttgtgagggtattatcaacctactggaaaatgtgacttaatatttgtttctcaaaaccgagaagaataatcaattcctaattttacttttcatagctttatttgttacgaaaaacaagaacgtacatgagacaatgtaatgataaatttatcactggtaagttgatcttgttctt

SEQ ID NO: 226 - DNA, 2836 bp, Arabidopsis thaliana
  source 1..2836: mol_type = other DNA
gagaaagatcttctttaacctctttgcaacaaatctactcaaaaccctaacccctttttcaaattacttccatctttagatcgaatcaaatcaatgtccgtcgacgatctccctccgtcgtcggcctccgccgtaacgccactaggctcctccgtgattccgattgtgaacaagctccaagacatattcgctcagctaggaagtcaatcgacgatcgctcttccgcaagtagctgtcgtcggaagtcaaagcagtggcaaatccagcgttctagaagctctcgtcggccgtgactttcttcctcgtggcaatgatatttgcacgcgccgacctctccgtctccagcttgttcagacgaaacctagctctgatggtggatccgatgaagagtggggcgagtttcttcaccacgatcctgttagacgtatctacgatttctctgagattcgccgtgagattgaggcggagactaatagagtatcaggagagaacaaaggtgtatcagatattccgattggtttgaagatattttcacctaatgttttggatattagtcttgtggatcttcctggtatcactaaggttcctgttggtgatcagccaagtgatatcgaagcgcgtataagaacgatgatcttgacttacattaaggaacctagttgcttgatacttgctgttagccctgctaatactgatttagcaaattcagatgcattacaaatcgcaggaaatgctgatcctgatggtcatagaaccataggtgtaatcacaaagttggatattatggacagaggtactgatgctcggaatcaccttctgggaaaaacaattcctcttcgacttggatacgtgggagttgtaaatcgtagtcaagaggatattttgatgaaccgtagcatcaaggatgctcttgttgcagaggaaaagttctttcgtagtcgtccagtttacagcggtctcacagatcgtttgggtgtccctcaactggcaaagaaattaaatcaggtccttgttcaacacatcaaggcactgcttcctagtttaaagtcacgtatcaacaatgcattgtttgctacagcaaaggagtatgagagctatggggatataacagagtctaggggtggtcaaggagctcttctcctcagttttattacaaaatactgcgaagcatactcctctaccctggaagggaaaagtaaagaaatgtcaacatctgagctctcgggtggagcaagaattctctacattttccaatcagtatttgttaagagcttagaggtaatcactgtgtgtagatatctttgtttagttagtggggctatttgcacggaaggcagacagttcgtttcaggttttcagttcttggaagttgatccatgtgaagacttaacagctgatgatattcggactgcaatacaaaatgcaactggtcccagatctgcattatttgttccagatgttccatttgaagttcttgtcaggaggcaaatatctcgtttattagatcccagccttcagtgcgctaggttcatcttcgatgagctagtaaagattagtcatcagtgtatgatgaaagagttacagcggtttccagtcctgcaaaagcgcatggatgaggttattgggaactttctgcgagaaggtcttgaaccttcgcaggcaatgatccgagatcttattgaaatggagatggattatataaacacctcacacccaaattttatcgggggaactaaagccgtggagcaagcaatgcaaacagtcaaatcttctaggattccgcatcctgttgcgcggccaagggatactgtggagcctgagaggacggcttcttccgggagtcaaataaaaacccgatcttttctcggccgacaagctaatggaatcatcactgatcaggcagttccaactgcagcagatgctgaaagacctgcaccagctggaagcacaagctggagtggtttctcatcaatttttcgggggagtgatggtcaggcagctgctaaaaacaacctattaaacaaaccattcagcgaaactactcaagaagtgtatcagaacttgtcaacaatctatttaaaggagcctccaactattttgaagtcatctgaaactcattcagaacaggagtcagttgagattgagataacaaagctattattgaaatcctactatgacattgtaaggaagaatgtcgaggacttggtcccaaaagcaatcatgcattttctggtaaattacacgaaacgtgagctgcacaatgttttcatcgagaagctttacagggagaacttaattgaagaactgttgaaagagcccgatgagttagcgataaagaggaaacgcacgcaagagactcttcgcattcttcagcaggctaatcggacgttagacgagctgccgttagaagctgaatcggtcgagagaggttacaaaattggttcagaagcgaaacatgaggagttaccgggcacaagaagatcaaggacagaaactaatgggaacggacggcttcatatgtaaattcaaattgtgattgcacgcatgactcattcatttggtgatggaaaagatttgttttcttccctttaaatcggttgtgcgtggggaaagaaaggtgttcttaggtatagtgaaacgtgatgataaacagaggttttgttgtgagggtattatcaacctactggaaaatgtgacttaatatttgtttctcaaaaccgagaagaataatcaattcctaattttacttttcatagctttatttgttacgaaaaacaagaacgtacatgagacaatgtaatgataaatttatcactggtaagttgatcttgttctt

SEQ ID NO: 227 - DNA, 3430 bp, Arabidopsis thaliana
  source 1..3430: mol_type = other DNA
aaactaaaataaataaggaaaacactcttcttcaaatccagtagagagattcaagacgagtctcatcgaaagttcgaatctttcgtcatctgattagctttctcttagggtttatttcgatttcgatttagtgttctccatcaatggcgacacccatggttagttccgctggaggattactagcaatgctcaacgagcctcatcctgttttgaagcttcatgcgctctctaacctcaacaatttggttgaccaattctggcctgagatctccactagtgtccccatcattgagagtttatatgaagatgaagaattcgatctgcatcaaagacagcttgctgctttgctggtctctaaggttttctattacttgggcgagcttaatgattcgctgtcttatgccctaggagccggtcccttatttgatgtttcagaggattctgattatgttcatacgcttctggccaaagcaatagacgaatatgccagtctcagatcaaaggcagttgagtcaaatgagatggtggacattgatcccaggctagaggcgattgttgaaagaatgcttggaaaatgcatcagtgatgggaagtatcaacaagccatgggcattgcgatagaatgccggagattggataagttggaagaagctatcattaagagcgataatgttcagggaactttatcatattgcatcaatgtttctcattcctttgtcaaccgcagagagtacagacatgaggtgcttagtttacttgtaaaagtctaccagaagctgccctctcctgattatttgagcatttgtcaatgcctcatgtttttggatgaaccgcaaggtgttgcaagcatattggagaagcttcttcgctcagagaacaaggatgatgctttattggcattgcaaattgcatttgatcttgtagagaacgagcaccaggcctttctcttgagtgttagagatcgccttccagctcccaaaacacgtgctgtagaagcaactcaggctgttgaaacaactattgctcctaatgagaacccttcgggagatgttcagatggcagatgaaactccagctcagacaattgtgcatgagacggatccagttgatgctacatatgctgagaggttaacaaagattaagggaattttatctggagagacatctattcagctgacactgcagtttctttacagccacaacaaatcggacctgctgatactgaagacaatcaaacagtctgtagagatgcggaacagtgtatgccacagtgctacaatatatgctaatgccattatgcatgctggaactacagtggacactttccttagagagaatttggattggctaagcagggcaaccaattgggctaagttcagtgctaccgcgggattaggtgttattcacagaggtcacctgcaacagggtaggtcattgatggctccatacttgcctcagggaggagcaggtggtggtgggagtccttattccgaaggaggtgctttatatgctcttggccttattcatgctaaccatggcgagggaatcaagcaatttctacgtgatagcctacgcagcactaatgttgaggtcattcaacatggagcttgtttaggtcttggcttgtcagctctcgggacagctgatgaagagatatatgatgatgtcaaaagcgtgctttatactgacagtgcagttgctggtgaagctgctggcatcagcatgggtttattattggttggaactgctactgaaaaggcaagtgagatgctcgcttatgctcatgagacacaacatgagaagataataaggggattggctctaggaatagctcttacagtatatggtagagaagaaggagcagataccttgattgagcagatgacgagagatcaggatccaatcattcgttatggtggcatgtacgcattggcattggcttacagtggaaccgcaaacaacaaggcaattcgacaattgctgcactttgctgtatctgatgtcagtgatgatgttaggaggactgccgttctggcacttggatttgtcttgtactctgatccagagcagactccccgtattgtatcactgctttctgagtcatacaatccacatgttcgctatggtgcggctcttgcagtgggtatttcttgtgcaggcactggcctcagcgaagcgatatccttgttggagccacttacatcagatgttgttgactttgttcgtcaaggtgctttgattgcaatggccatggtgatggttcaaattagcgaagctagcgattctcgtgttggagtatttaggcgccaacttgagaagattatacttgacaagcacgaagatacaatgagcaagatgggagcgattctggcctctggtatccttgatgctggtggtaggaacgttactataagactgctctctaagaccaaacatgacaaagtcactgctgttatcggccttgcagtcttcagccagttttggtattggtacccactgatctatttcattagtttggccttctcaccaaccgctttcatcggtttgaactatgaccttaaggtcccaaagtttgagttcatgtcacatgcaaaaccgtctttatttgagtacccaaaacccaccacggttcctacagccaataccgccgttaaacttccaaccgctgttctctcaacttcagttaaagccaaagctagggctaagaaagaagcagagcaaaaagctattgctgagaaaacatctggccctgaaaagcctgtcaatgaaagtggatctggaaaaggaaaagcatcaactgagaaggaaggagactctatgcaggtcgatagcccagcagcggtcgagaagaaagcacctgagcctgagccagcgtttgagattttagtgaacccagctcgagtagtcccagctcaagagaaatacataaagttacttgatgacagcagatatgtaccagtgaagctagcaccgtcaggtttcgttcttctcaaagacttgcgtgaacacgagccagaggttctctctctgaccgacgcaccaacttcaacagcttctcctgcaactggtacagcagcggctgcacaaggaaccccggcctcagctatggccgtagatgatgagccacaacctccacaagcctttgaatacgcttcatgattttatttcatttaaaattagtgggaatttgttgttggttttattggtttctgttgaaagacaaaaggcagagagaaaaacagatctttgaactttgagataactgcttcttgtcatttgcaactggtttcatgttcaactacgatcttgtattaggtacttctctttttaagtattatctaaataattaagtgtcgttttatgtcttttagtggtactggtatatctgatttcttctacaacaagatagtaataaactacattttctaagt

SEQ ID NO: 228 - DNA, 1570 bp, Arabidopsis thaliana
  source 1..1570: mol_type = other DNA
cctcttcctcttcaagccacaaagactcataaaaatccaacctttttctcatttcctctgttaaatttttcctgaaaagtttattcctttttctacagatcgaatttcgtagttctcacctcactgaatctgttactttctctataatctaaatcttcaaaaatgtttctggttttgtgaaatcaacacttaaaaactcgagcttttacctcctctgttttttcaaagataaaaatgagagaagacgattcagactggttcgcaagatgggaagaagaacttccatcaccagaagagctcatgccaatatcacaatctctaatctcacccgatctagctctagccttcgatatacgaaccccaaatcacggaaacggaaactcaaaccaacctcatcatcatcatcatcaccaaaccacacctcctacaccgtctcagcttcagcttccatcttctcaagccaattcctcagccgaattcgctgcagattcagcagatctaggctccggaggagctgccggagacgaaccagctcggactttaaaacgacctcgtcttgtctggacaccgcagctgcataaacgtttcgttgatgctgttgcgcatttagggattaagaacgcagttcctaaaacgattatgcagcttatgagtgttgatggtttaactagagaaaacgttgcgagtcatttgcaaaagtatagactttatcttaagagaatgcaaggtttgtcttctggtggtggtgctggttcagatccagctactgatcgtcttttcgctagctctcctgttcctgctcactttcttcatcctaatcgtgttcccagttctgatcatttcatgccgtcttttgttcctattgctactcttcaacaacaacaacaaatggctgctgctgcggccgctgctgcagcagctaatcctcatttacaaccgcctcagttccaccgtcaaatcgcagctgcgcattttggttcaccgactaatggtggattcagttcaccgacgagtaatggacagtttggatcaccgactagtaatggatttggatcaccgacgactaatgggaagtttgatccttcgttcttggcggtgaggcaaacgcagcagcaaccaattcagagaatgtctacaccatcattgcatagtccagttagtaattacgttgaggatttggaatctgcaaacgctaatggagggagaactgtcttaactttattcccaactcgagacgattaaaaagtcattatctttccggttactagatgttcaaaatgtctctgcagtcatcaagaacatgagtcagctgatcgttcttgtgcatcggttttatccggtggcgtttactctgtgcagtcttatcagtttaacattgatacaatccagcgtcaatgtgtggtcaagttctgtatctgaagctcaagcaccttcaggtcaaggcttgtattaacccttgtagctttcttgtgtaagattaatgtaaagatgagtttacttttgaacgtacatccgtacgaacaaactcaagattggaaaaaagaatgaaaaaaacaaaaacttacttcac

SEQ ID NO: 229 - DNA, 3856 bp, Arabidopsis thaliana
  source 1..3856: mol_type = other DNA
cgccggggactttgatatcatctgcgccgaacaaaaactctgcagaaaccctaattttcgtcagtgattgattcttccgtttccaccatggaagaagtatgcgaaggaaaagaattctcattccctcgtcaagaagaagatgtcctctccttctggactgaaatcgatgctttcaagacacagctcaaacgcactgagaatcttcctgagtacatcttctacgacggacctccttttgccaccggacttcctcactacggacatatcctcgctggtacgattaaagatattgtgactcggtatcagaccatgactggtcaccatgtcactcgtcggtttgggtgggattgtcatggcttacctgttgagaatgagattgatcggaaattgaatattaagaggagagatgaagttattaagatgggaattgataagtataatgaagagtgtaggagtattgtgactcgttatgttgctgagtgggagaaggttattactaggtgtggtcgttggattgattttaagaatgattataagactatggatttgccttttatggaaagtgtttggtgggttttttctcagctttgggagaagaatcttgtttacagaggattcaaggttatgccgtacagtactggttgtaagacacctttatctaattttgaagctggtcagaactataaggaagtccctgatcctgaaatcatggtgacttttccggttattggggatcaagataatgctgcttttgtggcatggactactactccgtggactcttccaagcaatcttgctttgtgtgtaaatgcgaagtttgtctatgtgaaggttcgtaacaagaacactggaaaagtgtacattgttgctgaatctcggttgtctgctcttcccactgataagcctaaggccaagttaagtaatggacctgctggtgatactaagaaagcaaatcctaaagctaaaggtgctaaacctgagagtgcagctgattcttatgaagttttggagaaatttaatggagcttccctggttgggaagaagtatgaacctttgtttgattactttagtgatttctcaagtgaggcctttcgagtagtagcagatgattatgtcactgatgacagtggtactggtattgtccactgtgctcctgcctttggtgaagatgattatcgtgtttgtcttttgaacaaaataatcaaaaagggggagaatctagttgttgctgttgatgatgatgggttgtttactgagagaattacccacttcagtggccgttatgtcaaagatgcagacaaggatataattgaagccgttaaggccaagggcaggcttgttaaaacgggaagctttactcactcatacccattttgctggcgatcagacaccccactcatatatagagcagttccaagttggtttgtgcgtgtggagcagttgaaagaaaagttgttaaaaagcaatgaacagacagaatgggtacctggttatgtgaaggataaacgttttcacaattggctcgaaaatgcaagggattgggcaatcagccgaagtagattttggggcacgcctcttccgatatggataagtgatgatggggaggaagttgtgatcatggattccgtagagaagcttgaaaagctctctggtgttaaggtctttgacctgcatcgtcaccatattgatcatattacaattccatctagtcgtggcgatgagtttggtgtgctccgtcgtgtagaagatgtttttgattgttggtttgagagtgggtcaatgccttacgcgtatatccattatccgtttgaaaacaaggagttatttgagaagaactttcctggtgattttgtggctgaaggtcttgatcagactcgtggatggttttatactcttatggtgttgtctactgcattatttgaaaaaccagcgtttaaaaatctgatttgcaatggtcttgtacttgctgaggatgggaaaaagatggctaagaaattacggaattatccaccacctttggaagtcatagatgagtatggggctgatgctgtgcgattgtacctgataaattctccggttgtacgtgctgagccactacgcttcaaaaaagaaggagtactcggtgttgtcaaagatgtattccttccatggtacaatgcatatcggttcctcgttcagaatgcaaagagacttgagactgaaggtggtgtgccctttgttcctactgatctggcaactatacagtcagcaaacattcttgatcagtggattcactcagctacgcagagtcttgttcgttttgtccgcgaggagatggatgcctatcggctttacactgtggttcctcgtctcttgaagttcctcgacaatctcactaacatatatgtcagattcaaccgcaagagattaaaaggccgtacgggggaagatgattgtcacactgctctttcgactctattcaatgtgctccttacttcttgcaaagtgatggccccgtttacacctttcttcactgaaactttgtaccagaatctgcggaaagcgtgtaaaggttctgaggaaagtgttcactactgcagtattccaccaagggaaggaatggaaggggagaggattgagttaagtgttacgaggatgatgaaaatcattgatcttgctcggaacattcgtgagcgtaacaaactacccttgaaaactcctctaaaggaaatgattgtagtccatcccgatgctgatttcttaaatgacataactggagtactaagagagtatgtcttggaagaacttaacgtgagatcgcttgtaccatgcaatgatactctgaagtatgcatcactaaaagcagaaccagatttcagtgtattgggaaagcgacttggaaagtcgatggggcttgttgcaaaggaagttaaagaaatgtctcaaaaagatatcttagcatttgaggaggctggggaagttactattgctaaccacttgctaaaggaaacagatatcaagattgttagggttttcaaacgcccagacgatttgaaagagaatgagattgattcagctggagatggtgatgttttggtgattctggatctacgagcagatgattctttggttgaagcaggttttgcccgtgagattgtaaacaggatccagaaactaaggaaaaagtcaggtctggaaccaacagactttgtggaagtgtacttccaatcattggacgaggatgaatctgtttctaaacaagttctagtctctcaggaacaaaacatcaaggattcaattggctccactttacttttgtctagtttaatgccatcacatgcagtcataatagccgatgagacattcaccccaaaagaaacatccgatgagagtgtcaaaaaggttccaaagttgtcgtataagataagtttggctagaccggctttgaagttcaatgaagaggctgttcttgctttatactcaggagatgtgaaatctgcaaccgggcttcaaacatacttgttatcaagggatcattcgaatttgaagtcagagttccaagccggagatgggaagataactgtaagctgcattgaaaatgtgcctgttgcgactgtggttcttggcgaacatctccatttgagtgtcggcgatgacttattgagcaagaggaacgcatgagccgtccctccggttttgtttgatttattgtggtaacttgggatttatttcccttatttttcttcttccatatcttacctataaactacttatttagattgttgcaaaaagttaaatctcagtgttgagactctcttttactcgttctatttgtacaatactaaacttttattcatctcaaaatttatggttggac

SEQ ID NO: 230 - DNA, 2933 bp, Arabidopsis thaliana
  source 1..2933: mol_type = other DNA
atcttctttctatagtttccatgaagtcttcttcttctcagagctacgatgttttccccaacttcagaggggaagatgtccgccactcattagtcagccaccttcgtaaggaacttgatcgcaaattcatcaatacattcaacgacaacaggatcgaaagaagccgcaaaataaccccagagctgttattggctatagaaaactcaaggatctcacttgtcgtcttctctaaaaactatgcttcttccacgtggtgcttagatgaattggtgaagatccaagagtgctatgagaaattggatcaaatggtgattcctattttctacaaagtagatccttctcatgttagaaaacagaccggagagtttggcatggtctttggggagacctgcaagggcagaacagagaatgagaagcgaaaatggatgcgagctctagcagaggtagcacatctagccggagaagatcttcggaactgataagtttcaggtgggtaaacttccgataagtgattgtcggaacagtgtgtatgaggattaaacacagagaaagatcgtgtggtgattgtagtgtcggtaaacaagtttttaaagcctcccagctgtaatgtatcaaccgtcggaaaggggtgggtcccacgagcctgagtaaagacgcgtagcgaagcagaaatgcttgaaaatatcgccaaggatgtttcaaacaaactcttccccccatcaaataatttcagtgacttcgtcgggattgaagctcatatagaggcattgatatcaatgttgcgcttcgactccaagaaagcgcgaatgatagggatttgtgggccttctgaaactggtaagactactataggaagagctttatacagcagactcaaaagcgacttccaccatcgggctttcgtagcatataagagaaaaatacggagcgactatgaccagaagctgtattgggaagaacaatttctatcagaaattctttgtcagaaggatataaagatagaggaatgtggtgcggtggaacaacgactaaagcacacgaaagttcttattgttcttgatgatgttgatgatatagagctactaaaaacgttggtaggacgtatcagatggtttggatctgagagcaaaattgttgtgattactcaaaaaagggaacttctcaaggctcacaacattgcacatgtttatgaagtgggattcccatcagaagaactggctcatcaaatgttttgtcgatatgcttttgggaaaaactctccacctcatggtttcaacgagcttgcagatgaagctgcaaagattgccggtaatcgtcctaaagctctcaagtacgttggttcgtcttttagaagactagacaaggaacaatgggtgaagatgctatcggagttccgtagtaatgggaataaactaaaaatcagctatgatgagttagatggtaaaggtcaagattacgttgcgtgtttaaccaatggttcaaattcccaggtgaaggcagagtggatccacttggcactaggcgtgtctatattacttaacatccgcagtgacggaacgacaattctcaaacatttgagctacaatcgaagcatggcgcaacaagcaaaaatatggtggtatgaaaatttggaaagagtgtgcaagaagtacaatatatgtggcatagatagctcaactgatggtggtggttcaacgtatggacaatgttcaaattcccaatttcaaagaaacatggacgcaagccccgggggaaacaaaaccagtaatcaatccacaaaagattcaccgagagcttctcaagtagagaaggaaaagatcgagtactgtgagccacatgtatatataacacctgccatcttcagtgacggaaccagagctcccaaatacgtggagtctagtagccgaagagtaacccaagtgcaccacgccaaaacatggtggccggagaattgtgaaaaagtgtacgagaaccacaacaacatatatggcatagatagatcaattgacggtggtgataagtttgagggaaaaagtaaagttagtgacggtgggttagacggcaaagatcaaggttcaatgtatggacaaagttcaaattccgagcttcaaataaacatggacgcagacaataggagatgtgaacctgtgagtgagatgcttttcaaaaattacaatgtgtgttcgcctaatgcacatatgcctgcaggtttaaccgatgtaaactgttcaaatccccagtctcaaagaaaattggacgcaagcctaaagaaagacaaaattgtgcatgagtggattcgaaccggtagtggcttcttttttgattttcagggcccaaaatctatagtgagtgctgctcaagtggacgagaaaaatttcgagtactgtgaacaaggtgtgtatataacacttggcatcctcagtggcggaattatagttctcaaacatttggagttcagccgaagaatggcgcaacaagcaaaagtttggtggtcggaaaattggataaaagtgtaccaggagcacaacatatgtggcatagataaatcatttgatggtaggtttgatgatagacgtgtcattcgtcaattaagaccaaactgaagcacggcctggcccttgtaaaagtcaggacttgagctagatacttaagccaagcagatctgtttggctaagtctggtcgggttagcgcgaaattgtcgaaatctgtttaataattgcattcagtgcaagtctttaatttgtacttcatggctttaatacactaattaaacattggatcatttttagttttttcttcttttcaaaatacatatttttctaagttttccaatttagtgattttattagttctttagtagaatactggatcatggacacatgctacaacatgctttgacctaattatgaaatttatcagcgaatatattatg

SEQ ID NO: 231 - DNA, 960 bp, Arabidopsis thaliana
  source 1..960: mol_type = other DNA
tcgtcgccgttaaaatctgcgatctcattgctcattggtgaagacaaatccctaattcgtatgtcagattctgtggccggcgatttccctcccgatccacttctcgcctccggagccttcatctcctccgccggtgacggaaccctagacagtagtgcgaaacgacgaccgattcagggaggaatcggaatttctggttcgggagagagcgtacgcatcgggatggctaacggcactgatcaggtgaatcatcagacggaatcgaagtcgcggaagcgtgcggcgcctggagataattggttgcctcctggttggagagttgaagataagattcgaacttccggtgccacagctggttcggtggataagtactattacgaaccaaatacaggacgtaagtttcggtccaggactgaggtgctgtactacttggaacatggaacttctaaaagaggcaccaagaaagctgagaatacatatttcaatccagaccattttgaaggccaaggaagcaatagagtcaccagaaccgctacagtgcctccacctccgccaccgccattggactttgattttaagaatccaccagacaaggtaagctggtctatggcaaacgcaggagaagaaggttggatacctaatattggggacgtcaaggttcaagactcagtgagacgagattggagcactgcgtttacctttatcacaagccgaaaccctagtaaagtgtcggcttgaagactatgatgacctggtggcgatttttgggttgttgcatctatggtaagatacacagatatgttagggttacttaattggataattcttgtttagtcttcattcatcaatcatcaagttatgtgtactataatttttgtagcggtttttggaagaatatgataaggggatatgcatctttgtgtttgcttttggaacgtgataaccattcgacttttgact

SEQ ID NO: 232 - RNA, 464 nt, Cardiovirus A
  source 1..464: mol_type = genomic RNA
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 233 - RNA, 228 nt, Tobacco mosaic virus
  source 1..228: mol_type = genomic RNA
atctaagttaggttgtaaacatattagagacgttgttcatttggaagagttacgcgagtctttgtgtgatgtagctagtaacctaaataattgtgcgtatttttcacagttagatgaggccgttgccgaggttcataagaccgcggtaggcggttcgtttgctttttgtagtataattaaatatttgtcagataagagattgtttagagatttgttctttgtttgata

SEQ ID NO: 234 - RNA, 121 nt, Hibiscus chlorotic ringspot virus
  source 1..121: mol_type = genomic RNA
tagcttatttgggttctttatcactgtcctgattaaggctgaatcagaacaccatcacttccactcacacgacagtagtaagacacagaacatagtagttaatactggaaagtaacccacc

SEQ ID NO: 235 - RNA, 147 nt, Zea mays
  source 1..147: mol_type = mRNA
agcacaacatttcaaccagaaacactagccgaagcaaatccattccacaagcacctggtgggatcatctcatcatcagaaaccaagagagagattccgtgtccgcttgttgtagtagattgtgaggactgaggaccgagaagcagcc

SEQ ID NO: 236 - DNA, 130 bp, Arabidopsis thaliana
  source 1..130: mol_type = genomic DNA
gtaaggatttttatgatatagtatgcttatgtattttgtactgaaagcatatcctgcttcattgggatattactgaaagcatttaactacatgtaaactcacttgatgatcaataaacttgattttgcag

SEQ ID NO: 237 - DNA, 91 bp, Arabidopsis thaliana
  source 1..91: mol_type = genomic DNA
gtaacattccttagttacctttcttttctttttccatcataagtttatagattgtacatgctttgagatttttctttgcaaacaatctcag

SEQ ID NO: 238 - DNA, 108 bp, Arabidopsis thaliana
  source 1..108: mol_type = genomic DNA
gtgctcttgaaattggaactcttcttttgttgtctaaacctatcaatttctttgcggaaatttatttgaagctgtagagttaaaattgagtcttttaaacttttgtag

SEQ ID NO: 239 - DNA, 101 bp, Arabidopsis thaliana
  source 1..101: mol_type = genomic DNA
gtaagttgttacttatgattgttttcctctctgctacatgtattttgttgttcatttctgtaagatataagaattgagttttcctctgatgatattattag

SEQ ID NO: 240 - DNA, 121 bp, Arabidopsis thaliana
  source 1..121: mol_type = genomic DNA
gttagtatcatatgaagaaatacctagtttcagttgatgaatgctattttctgacctcagttgttctcttttgagaattatttcttttctaatttgcctgatttttctattaattcattag

SEQ ID NO: 241 - DNA, 124 bp, Arabidopsis thaliana
  source 1..124: mol_type = genomic DNA
gtaaatcctggtccacacttttacgataaaaacacaagattttaaactatgaactgatcaataatcattcctaaaagaccacacttttgttttgtttctaaagtaatttttactgttataacag

SEQ ID NO: 242 - DNA, 136 bp, Arabidopsis thaliana
  source 1..136: mol_type = genomic DNA
gtaagaggtcaaaaggtttccgcaatgatccctctttttttgtttctctagtttcaagaatttgggtatatgactaacttctgagtgttccttgatgcatatttgtgatgagacaaatgtttgttctatgttttag

SEQ ID NO: 243 - DNA, 150 bp, Arabidopsis thaliana
  source 1..150: mol_type = genomic DNA
gtaagttctgcatttggttatgctccttgcattttaggtgttcgtcgcacttccatttccatgaatagctaagattttttttctctgcattcattcttcttgcctcagttctaactgtttgtggtatttttgttttaattattgctacag

SEQ ID NO: 244 - DNA, 121 bp, Arabidopsis thaliana
  source 1..121: mol_type = genomic DNA
gtaaagcaactgtgttttaatcaatttcttgtcaggatatatggattataacttaatttttgagaaatctgtagtatttggcgtgaaatgagtttgctttttggtttctcccgtgttatag

SEQ ID NO: 245 - DNA, 136 bp, Arabidopsis thaliana
  source 1..136: mol_type = genomic DNA
gtaaagtttccaactttcctttaccatatcaaactaaagttcgaaactttttatttgatcaacttcaaggccacccgatctttctattcctgattaatttgtgatgaatccatattgacttttgatggttacgcag

SEQ ID NO: 246 - DNA, 121 bp, Arabidopsis thaliana
  source 1..121: mol_type = genomic DNA
gtctgtctttcctatttcatatgtttaatcctaggaatttgatcaattgattgtatgtatgtcgatcccaagactttcttgttcacttatatcttaactctctctttgctgtttcttgcag

SEQ ID NO: 247 - DNA, 159 bp, Arabidopsis thaliana
  source 1..159: mol_type = genomic DNA
gtaaaatattggatgccagacgatattctttcttttgatttgtaactttttcctgtcaaggtcgataaattttattttttttggtaaaaggtcgataatttttttttggagccattatgtaattttcctaattaactgaaccaaaattatacaaaccag

SEQ ID NO: 248 - DNA, 109 bp, Arabidopsis thaliana
  source 1..109: mol_type = genomic DNA
gtaaggacttctcatgaatattagtggcagattagtgttgttaaagtctttggttagataatcgatgcctcctaattgtccatgttttactggttttctacaattaaag

SEQ ID NO: 249 - DNA, 82 bp, Nicotiana tabacum
  source 1..82: mol_type = other DNA
agacgcatccatggctgaatggttaaagcgcccaactcataattggcgaattcgtaggttcaattcctactggatgcaaaga

SEQ ID NO: 250 - DNA, 59 bp, unidentified
  misc_feature 1..59: note = unclassified Partitiviridae
  source 1..59: mol_type = other DNA
acttgtccacaatcttttctgatgagtcgctgaaatgcgacgaaactcgatatgttcaa

SEQ ID NO: 251 - DNA, 68 bp, Hepatitis delta virus
  source 1..68: mol_type = other DNA
ggccggcatggtcccagcctcctcgctggcgccggctgggcaacatgcttcggcatggcgaatgggac

SEQ ID NO: 252 - DNA, 43 bp, synthetic construct
  misc_feature 1..43: note = synthetic construct
  source 1..43: mol_type = other DNA
ttttacctgatgagtccgtgaggacgaaacgagtaagctcgtc

SEQ ID NO: 253 - protein, 10 aa, synthetic construct
  REGION 1..10: note = synthetic construct
  REGION 2..5: note = misc_feature - Xaa can be any naturally occurring amino acid
  SITE 8: note = misc_feature - Xaa can be any naturally occurring amino acid
KXXXXILXFY

SEQ ID NO: 254 - protein, 12 aa, synthetic construct
  REGION 1..12: note = synthetic construct
  REGION 3..4: note = misc_feature - Xaa can be any naturally occurring amino acid
  REGION 6..7: note = misc_feature - Xaa can be any naturally occurring amino acid
  SITE 10: note = misc_feature - Xaa can be any naturally occurring amino acid
IVXXVXXVLXNL

SEQ ID NO: 255 - DNA, 8853 bp, synthetic construct
  misc_feature 1..8853: note = synthetic construct
  source 1..8853: mol_type = other DNA
taagtcgtcttcatcggcgggttataattacacaggcaggaaaggccttattggtgatgaaaatcactcaagggcaatttcgatagctaaagctgtattatggtcagccatcaaagatgatggagaaggcattgaacatgtcattcggacttcagtaccagacgttggctacactcgaactcagttaacggacttgctagaaaagacgaaagtcagacaagtttggggcagagcattccactatatcctacttgaaggattggttgcatacccgtttatacaaacagtcatgtcacataaaacgtttattcacgcaggacaagacccgttaattagtgttcctagattattatcagacgttgcattgaattgcaaatggatttactcacttgattggagtcaattcgacgctacagttagtcgattcgaaatccacgccgcttttgacataattaagtcttacgtagactttccaaattatgaaacagagcaggctttcgagatcactcgacaactattcatacataagaaagttgcagtacctgacggttacatctacgaatcacacaaaggaattccatctggaagttactacacttcacttgttggatcgattatcaattatttgagaatcaactacctctggagactcctcaccggacacccaccacaacagtgtcacactttaggagatgactcattggttggagataactcatatgttaatccacaagcgattgaagaagcagcaaacaagctaggctggcacttcaacccagataagactcaatactctacagtacctgaagagatcacatttctcggacgaacttacgtaggcggattaaataagagagatctaacgaaatgtattagattactcgtatatcccgaatacccagtagagtcaggtaaatcctggtccacacttttacgataaaaacacaagattttaaactatgaactgatcaataatcattcctaaaagaccacacttttgttttgtttctaaagtaatttttactgttataacaggtcgaatttcagcttatagagctaagtctatcgcacaagatgctggcggactcagcgaagtgctaaataggatagcagataagcttagacgtatttacggtacggcttcagaagaagaggtaccaatttattttaaacgctatgtatttggagtttaacgtaataaatacgtggaaggtatttaaaccttaccggccggcatggtcccagcctcctcgctggcgccggctgggcaacatgcttcggcatggcgaatgggacgcacctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattataaatccaaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaatgcgaacagtatgctttacagaagaatcgagctggtaacagtatgctttacagaagaatcgagctagacctgatccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaattctctgatgagtccgtgaggacgaaacgagtaagctcgtcagaatttcccccttgttccaaggcacctctcacgctccggtgccataaacaagagattgaaattaatcaatttatatacttacctcgttacgaatggtaagaggcacacttgttggttatgactatacccaatttcagggtgatttagttaaatcaactcaccgacaccctcatgttgttcaccgcgaaatcgcaacaacatatgtcgatcagtatgcttatgagcatattgaaacattcagtagtttatatcctgaacttatccttaaaggatggtctaggagttactatctgcccgagaagcatctcgctgcagtgttaaactacagtatgccaaatgttcctgcatctcagctcagtcagtcattatacagacaagcgattgaatcagctaagaatggatttattagccttccccgagtgaaggctttcgatgtacttaccgaaatggaccaagtaccttt

SEQ ID NO: 256 - DNA, 8797 bp, synthetic construct
  misc_feature 1..8797: note = synthetic construct
  source 1..8797: mol_type = other DNA
gcgaacagtatgctttacagaagaatcgagctggtaacagtatgctttacagaagaatcgagctagacctgatccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaattctctgatgagtccgtgaggacgaaacgagtaagctcgtcagaatttgacctgaaccccatatcaggatcgaattatttacaaattcaccaaacacgtctaagcttgtgaaaattattgttatccctacattaacatgggcgatcgtgttaacgctcaagacgacgatacagtcgtccctcaccaagcacctcttcaaccagcagctcttcagcaagacctcaccagatctgccgactacttattagacaacgtccgtattggcaaccaccgacaacgctatgataaataccgtcgctatgtccttctcagatccagcgaaatattcactagtctcgtcgccatttacgctcacatcttttctagttattggcaacactttcggaggttcactgatcaatttcaagctccaactggagtccaactccccacttttgtggcacgagtctacatctccacttggcttcatgatctatactgctctattcgagaggccactagaagtatttctccattagcctttaatgagaggtactcatatgaattgctcccatactccaccgagtacgacccctttctcgcatttctgtctatgtctattaaaccaacacatattcagcatactcccgagaacacattatggattcctatcctttgtgaaaattacgactgggatcgaaacgaagcaaatcacaacccttttggcattactaatttcacattgaatagtaacctattctatgggcttttagcgatactgaaagaacggaaggagtttaagttatcaactctgactactaatacaattggccgaccctgttggctctttgattggcacgacaacgtgcaagtctgcgcatggtttccccgtgaagcaaacttcaacagtcaagatgtaaccgccgcttacatcattggagtagcctgtactcccaaactaggacccagcgatgacgacgcatggaaatactatgcatccctcaactctgtaccaactttcacaccaactgagccgcgtctcaccaatcgtcggtcatacggtgcttatgaagtccgcactcgtgaaactgaaaataactatttcttaccagatagtcttctcaacatcatcgaggattttaccgcaacaggtaaatcctggtccacacttttacgataaaaacacaagattttaaactatgaactgatcaataatcattcctaaaagaccacacttttgttttgtttctaaagtaatttttactgttataacaggtacaacacaacgaaggaagatccgtcgcccttcagctacgtccgcgtctaccggagccgctatcattatcagagatacgcccggaacagcgtccactgcaaccacgtccactacggagacggaagttaccttccctccagtcatcaggactaagattagagactggtactaccactctcgtgtaattctagaacttgaagacaattctaggacagcagctttaagaatgtttatcattgcttaattagctttcctttatgcttttgtcatgttataaacaatttcgtttaattttaatattaatcgcagttaattttatacatgtgctctgttatttgatatcagttccgcataatggacaggctacaatacccttcagtagggtcacaaacgtgacccaggaagctgtattgcaccctgctggccggcatggtcccagcctcctcgctggcgccggctgggcaacatgcttcggcatggcgaatgggacgcacctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattataaatccaaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaat

SEQ ID NO: 257 - DNA, 9251 bp, synthetic construct
  misc_feature 1..9251: note = synthetic construct
  source 1..9251: mol_type = other DNA
gcgaacagtatgctttacagaagaatcgagctggtaacagtatgctttacagaagaatcgagctagacctgatccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggagttgtctgatgagtccgtgaggacgaaacgagtaagctcgtcacaactcctgctcaagcttttgacaatcaaaatagaacgtcaatctcagttgctctatctcaacttgttacctactctacatggattacctcacttccgcatttaaccgaatcactcattggttcaccaaacccaccaatttagaatacattggagactactctacacctcctagtatcatccgcgtcaatgaagttgctattgctaaccacaagaaaacacttcgctacgcctttgaacagtatctgtacgatcacgaataccgtatgatcgttgatgaatatcgtcgctcagacatagaccaagagtctatcctcgccgactttttctctggtgatattgaaccctttgatatacctttcgacgaacacgtcgaacatggactacgctgtatggccaacgccttcagacctcctagaccttgccgccctgctcacatcctagatgtcaagcacggatacccttacaaatggaacgtcaacgcagaaccacccttctcaactgacccttacttcctttctcaacgtaagacctttggcgagttcattcaaatgcacgaatatgaacacatcgataaagctgacttctttcgaagacatcccaacaccgaatcacacgacttcctcactacagtggtccctccaaaatttggatacctcaagtcgatggttttctcctggacccgaagatggcaccacgtcattaaaaccggattccaagacttaactggactcgaaaactctggatatctctacaatcgttttatctttcccatgctcctacacaccaaaactgctattgtcaaaaagaacgatcctaacaagatgcgcaccatctggggcgcccctaaaccttggatcatcgctgagacaatgctatattgggaatacatagcctggatcaaactcaactccggtaagacaccgatgctttggggatttgaaaccttcacaggtggatggtttcgtttgaacaacattttgttctgcggactcatcaaacaatccttcttaacattagactggtcacgcttcgacaagcgtgcctatttcagtcttctccgacggatcctttacacagctagaaccttcctcactttcgacgaaggctatgtcccaacacatgcagcaccgacccaccccaagtggaaccaccaaaaagcactcaaccttgagcgcctttggctctggacactcgagaacctctttgaggcacccatcatcctacctgatggccgaatgtacagacggcacttcgccggaattccttcaggtctatttatcacacaactattagactcttggtacaattacacgatgctagccacactcctaagcgcactgggcttcgaccctaaatattgtattattaaagtgcaaggcgacgactcaattatcaaattgacgaccttaattccacgtgaccaacacgagaacttcatggtccaactcaccgccctcgcaaccacgtactttaatgccaccatcaatgtcaagaagtcggaaatcagaaacacgctcaacggatgtgaagtcttatcatacaagaaccacaacggtttcccccaccgtgacgagatcactatgcttgctcaattctaccacaccaaagcacgtgacccaacgcctgaaattaccatggctcaagcaataggctttgcctatgccagctgcggcaacaacaaacgcgtactgtggattcttagagacatttacaacttttacaagatgcgtgacgtcacccctaatcgtgcaggtaaatcctggtccacacttttacgataaaaacacaagattttaaactatgaactgatcaataatcattcctaaaagaccacacttttgttttgtttctaaagtaatttttactgttataacaggtttaaccctcactttcggagactcaccagacgttttcatgcctgaaatccctcttgatcattttcctacaataactgaaattcggagatacctaacatgctctgaatatcgcaacgaagcacaagacgctagaacttggccgagatcactattcattagtggtcctgccgaataaagccgatcgctttgattgtttaaaattaaaaaaaaggccggcatggtcccagcctcctcgctggcgccggctgggcaacatgcttcggcatggcgaatgggacgcacctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattataaatccaaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaat

SEQ ID NO: 258 - DNA, 8461 bp, synthetic construct
  misc_feature 1..8461: note = synthetic construct
  source 1..8461: mol_type = other DNA
gcgaacagtatgctttacagaagaatcgagctggtaacagtatgctttacagaagaatcgagctagacctgatccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggttaatcctgatgagtccgtgaggacgaaacgagtaagctcgtcgattaacagccttcataacgctttttgcaatcaaaatagaacgcaaatctcagctgctctaactcaactttttacttactctactgcttacccaagccgcccacaccacaatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcagaggtgaaccttcttaaaggagttaagcttattgatagtggatacgtctgtttagccggtttggtcgtcacgggcgagtggaacttgcctgacaattgcagaggaggtaaatcctggtccacacttttacgataaaaacacaagattttaaactatgaactgatcaataatcattcctaaaagaccacacttttgttttgtttctaaagtaatttttactgttataacaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtaatgatcggtcagtgccgaacaagaactatagaaatgttaaggattttggaggaatgagttttaaaaagaataatttaatcgatgatgattcggaggctactgtcgccgaatcggattcgttttaagcatccatggctgaatggttaaagcgcccaactcataattggcgaattcgtaggttcaattcctactggatgcagtttggacccagaagtccttaaactctaacccattttgctgtcaaacagtctatctgattagaaagggttttttttgctttaacttaacttttatctttaattttctctttcctatactgattcctcgcttcctcccgaagcgtattcttggaagctcagcttccgacctaggaacaaaaaaaaaggccggcatggtcccagcctcctcgctggcgccggctgggcaacatgcttcggcatggcgaatgggacgcacctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattataaatccaaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaat

SEQ ID NO: 259 - DNA, 9047 bp, synthetic construct
  misc_feature 1..9047: note = synthetic construct
  source 1..9047: mol_type = other DNA
gcgaacagtatgctttacagaagaatcgagctggtaacagtatgctttacagaagaatcgagctagacctgatccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggttaatcctgatgagtccgtgaggacgaaacgagtaagctcgtcgattaacagccttcataacgctttttgcaatcaaaatagaacgcaaatctcagctgctctaactcaactttttacttactctactgcttacccaagccgcccacaccacaatgaatcctgaaacaccacttgccgaccccaacggtcccaccccacctcacggtgctgctgcccaacccacccctggccaacacatccctgctggaactccctccactggtgctgtccacttgacaccagccccgcaacccaagcgcaaccgcgcgccccgtggcccgttcgacaatgcctcgagcacctctgctcctggagctcctgctttactggagcttgctgctgcctaccccatgtccacggaacaaaggcgttccccaaacaccttcgtccctgacgcccagatgcttttccatgtcctcagcttatgcgacaacatgatgctgtctaccgaccgtttcacccgctctgctcccgcctggctccccatcgtctcgcagctttacatttccatcctttggttcaccatgattctcaaagtgtatgtcaactcctcctatggagccatgcacgctcacctcctgaatatcttagtcgcccatcttcgtatagatgagtgccttattcctggacctctcgtcccattcttccaatctcttggagccattaacggcccttttgaatggatcggagacatagtgccgatcatccccgaattttctcagctttggaacaccacatcgttctaccccactgactcattcgctcgcatcttccccatccctgctatcctcctcgatcaactgcactactttgcctccatcacagtcaccgaacaagaaaccattttcggtcactttgaatggtacagaaacattttctctctcggccaaggcaccttcaacccccgccttcgcctaggcccgcaactctgtggctccctctatgccaccgaatctcaggtaaatcctggtccacacttttacgataaaaacacaagattttaaactatgaactgatcaataatcattcctaaaagaccacacttttgttttgtttctaaagtaatttttactgttataacaggtcaccaacgctcgcactttttggagcgctgccctccgcaccgggataacccgcaccaatgccgcagctaatcaacctctttttagctcctaccctcaactccttggtttcgtcgaccagaacgacgctatgcagctggactggttccagcacacagccatagtcatgcagaagtactgtcagtacttcaatggctccgttcccctcaaatcaatcagccccactggcatcggcgctgtcgccgttcacggctaccccaactacagcggtgctgctcgcacctggatgtaccctgctaccaacaccctctcctcattcaagtctagccgcttctccgccctcagagaaatccctgctctcctcaccgttaggttcgagcatgctgatcacgagcttgaagaacaagccgaacagtatgccattgccacacatactaacatgtgctggtctaagaaccttacaacccagaacaactggactcaagtagctgaagccaatgtgcactcaggcacttactggtctatgacccctcatcgggcacacgcctctgtaaggctgaagacacaatacgcccagctcgtggcctcccgttatcaccaacaggctgccaaccgtgtctaagtttggacccagaagtccttaaactctaacccattttgctgtcaaacagtctatctgattagaaagggttttttttgctttaacttaacttttatctttaattttctctttcctatactgattcctcgcttcctcccgaagcgtattcttggaagctcagcttccgacctaggaacaaaaaaaaaggccggcatggtcccagcctcctcgctggcgccggctgggcaacatgcttcggcatggcgaatgggacgcacctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattataaatccaaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaat

SEQ ID NO: 260 - DNA, 1708 bp, White clover cryptic virus 1
  source 1..1708: mol_type = other DNA
gttttaacttaagcttttcgtaatcaaaatagaacgcgaatctcaactgctctacctcaactttttacctactctaccgctaacttagccgccctacccacaagatgaatcaagacactcctctcgccaatctcaatggccccgaagttccatccggcaacgtccctcccgccaaccctcctggtcgaacaaacgtcgcccctcccgcccaaggtgctgttcaacagcctcctgctccggctgctcgtcgtgcccgcaacccacacggccctgtccctcctgctggaccatcccgttctgctggtgcccctgctatgcttgaactttccgctgcctaccctatgtacaccgaacaacgtcgtgcacccaactactatgtgcctgacgcacaacttctcttccacaccctcggtgtctgcgaccaactgatgaccacaactgaccgcttcctccgctccatgccttcatggttgccaatcgtctctcagctttacgtctctgtcctttggaatgttatgatactcaaggtctatgtcaacaccggttacggtgctgcctacgcacatgacttggatgtcctcctcaaccaccttcaaattaacgaatgtatgatccctggccccctcgtcccctttttccaatccctcgctgccgtcaatggcccttttgactggataggtgatattattcctgccatgccatcattcactgaactctggactgatgaatttgctccccatgctgcttacgctaggcaaatacccatacctgccatcctccttgatcagctttaccgttttgccactcttgcctttgatgctcaactacaaacaaactatgccacttttgaatggtatagcaacattttcaaccaagatgtcaacactcacaatgctcgtctccgcctcggccctcaactctgtggctctctgttcacaacacaagcccagtgtgactccgcccgcgccttctggaacccagcttttgctaatgggtttactagaatcgatgctgctaatggccccctaatggcttttccccaactacttggatttatttctcaagatggtgccctgcaatctaactggttcatgcacatctccttaatcatgcacaagtacgctcagtacttcaatggttctgtaccccttaagtccatctcccctgttggaattggtgcttccgtgatctacggaactccgcttgaagataccaacgtccgtgactggctctaccccgctgctgctgctattgctcctttccgttctacccgctttttaccgcgtcgtgaactacctgcgaccctagctgtccgtttcgcccatgccgatcatgagatcgaagaacaagctgaacagtattccatactgtgtcacaccaatatgaagtggtatgtcaacaacgccactcagaacaaccacacagctattgaaggaaactacattcaccaaggtgagtactggaacttcacccctttccgctactctcctcctgtctctctgaagacccagtttgctcaagtgatcgcttcccgctaccaccaacaagctgccaaccgtgctgaatgattcctccgacctttgatgtcgttaatctcaccaattttgctgcctaagtgcagttaatctgatcagattggattttttttgttttaactttcttttaatcaactttatccttttaattctgacctattatcgctcttgcg

SEQ ID NO: 261 - DNA, 1779 bp, Vicia cryptic virus
  source 1..1779: mol_type = other DNA
gatcaaaagagtaacagtccttgtaccaacttttcacaatcaaaatagaacgcgaatcttgactgctctacctcaactttttacttactttaacgttgtttatcaaccgtccctctcaatggaagctcatactcccgctgctgatgtcaatggacccaatatcccctccggtgctgtctctcagccagaaatcgctccacacaatcaagctgcccccaacgtctctggtgctgctcaggctataatagcccccacccctgctcgtaagcgcacgcctcacggacccattcctgctgccacctcatctggcaacacctctgctcctgctctcctggaattagctgctgcttatcccatgtacaccgagcaacgccgttcattcaactttttcatcccagactcccaaatgatgtttcactctttaggtatctgcgactcaatgatgaactccaccgatcgttttcttcgatcctcacccgcttggctccccatcgtttcacaactttacatctctgtactgtggaatgttatgattatccgcatcctctctcacactggatatgccccttccttcaccgatctactcaatactctcactactgaccttcaaattgaagagtgtatggtccccggaccactagtccccttcttccaatccttggcctctaccaatggcccttttgactggataggcgatatcactcctgccctccctggatttgattctctctgggatgccacgaatttcactccgcacgcaagctacacccgcttcgtccccattcctgccatcttgttagaccagctttactactgcgcgacttacgtcggcgttaatcaaggcgacctctaccctaccttcacttggtatagaaacattttcacccgcactggtgcaaacactcctgctctcctccgcatgggcccaaatctttgtggctcacttttcaccacctccaatcaattcgatgctgcccgtacttattggcgcgcctgctttggcactggttttacccgtgttaatgtcaccgctgctgccttcaccaactacctacagctgttaggtctccgttcccaaactggagaaccgcagactgcctggttccagaatgtgaccatggttatgcagaaatatgcacaacacttcaatggttccgttcccctcaagagtatcgacctcactggcattggcgccgtcgcccttaccggcacaccagttaataatactgctgtccgcgactggctctatcctccaaatgccaatatcgaaccgttcaccactggtcgattcaaccctcgtcgtgaaatcccggcccaactccgcatccgcttttctcactccgatcatgaactcgaactccaagctgaacagtacgctattgctgcgcacaccaacattaggtgggctgctaacatagctacacaacatgaaaggaccgccatcaatccggctcatcttcaccaaggtgattactggaacatgacccctttccgtcacaccggacatctcggcctcaagactcagtatgcccagctcatcgcttctcgctatcaccaacttgccgccaaccgtgtcgactgacaacctcctgacctagaacgtcaataaacttctaattttgctatctttgatagttattctgatcagaatagttttttgtttttttccaactcccaacttagttttctttaatacaaatttttatcctctctgacattttgttcgcccaaagtaaaaaaaaaaaaaaaaaaaaaaaaaaaaaaaaggaaaaaaaagtt

SEQ ID NO: 262 - DNA, 1791 bp, unidentified
  misc_feature 1..1791: note = unclassified Partitiviridae
  source 1..1791: mol_type = other DNA
agaaaaattttagaacctactctaattgatttttctaagctttatttcttttcctctgaaaaccaactttcaacataatggctcatcgtactcccactaatgccccggctcttcctcaagttgatgggatcaaccccaaccttccccccaacgacccaggaaccgtcgccgccgcccctaatgctaggaacttgcatctcgaaagagaacaagaagctagacaagaccgcgccgccgccgccttcgtccccaacactcgctttgcggtgagaagaccgacgattcgcccctccccggcccactctgcccctgacctagagactgctctctttgattcagccgtcaaccacccccccaccccggtctcctatccgtctacctcatcctacatccccaacttcacttccgccttctactatctcaacaagatggactccttgatggtccaaactctcaactggacaaacaactgctctggatgggttcccccatactcccagatctacatcagcatgcttctctatcttcaagtaatgcgcgccatgaagaaagctggcgtcctcaggcccaactctgaactatcccatctcttcaatgagatgtccacaatctttcctttcgaaagtctgatggttcccggccccctcgtcaacttattcgagaatatcactgcctttcgtcctcttcaaacggactcttttggcaatgtgactcccttccttccagccgaacccggttggtcaaacgcgaccttcttcgctcccaacggctcattagttcgacacctccctcacatacccgctttaatctcccgtctccggagaatttgcgaaaccgcttccgagaatggcttaaatgacatctcgttttccgcccaccaccacggcccggagttcatatcagaactttttggccacatttgtgacaacgatcttcctgaacagctactgctgctcacccctggcctggccacctcctactctggtacactctacctctggcgccaagcccgctctcaactacaaaggtccctcttccctgaggcactcactgtcaacgacgtagtgcccaatacgtggacctccttcctctgcctggacaatgatgattcatggttcagtcccctcgccgctatgatgaacaagtactgccaatattggcatggctcagctcccctctcctccatccccgctgatggatccgctgctggctcaatgatttgcaatgagctcaatggatcctctatttaccggctggctcaatggaccgctcctattatggacccccccgctgacgaggatgccccacctgtcattgccgaacctggctactactaccccaatcccaaccccgtcctcatctttgacgctcgtacctgcattgaagacatctcttctgcacacatgttctccgctatgaccttccatcccaatctcataccatacggagctaacagagctaactttctgaaaggacagttttggaattgccaccccccaagctacagcgctcctgcccaccaggtctatccagctgtatccgcccttatcgctcgtgagtatcacagtgaaactaacctttcatctgacaagtgactctctcgacacccccgacacccctctcgctgatgacgaagatgtgaccggagtagacgctagcgcgttccttcggaatttttcggttcttttgttttgtcgttagctacgactttttcttatttcaatgtaaaccctacctattagctatcttttaaattaaaagaaaaaaaaattaaaaaaaaaaaataaaacc

SEQ ID NO: 263 - DNA, 1465 bp, unidentified
  misc_feature 1..1465: note = unclassified Partitiviridae
  source 1..1465: mol_type = other DNA
gataatgatcctgggaaagatcgttattattttactagtgtaaatttcttaaagagcttttcttcaaggtaattttcactttctcactttccccttagatgttatctaagcttgccttgttataataatttataactctactagtatgtttactatcttactttcgtagatatggctagcccacgtgactctatgatgacgactgctcaagacgctaagaaacgtaaagtttctcatattttgagtccgattcgaatgatcgagcctgctgctcaatccctcgcaatcatcccagttgtccctcaagtccaaatggatgacgctgaagctatggctgaattgctgtatcgtgaacgcgcccgcattgaacttgatattaatcctgcttatatcagtagattggaatcccgtcgagagattggtttctatcctcctgttgcaagtaagaacgttccgttcactactttcaacatcgatgtcgctgaattcagacgctgcataacacatgtacttactgctgcactcaaagtacgttattacgatcgatcaaatcttcgcaacgctgagatcaatactgagataaacacgattgtgcctcgcactgtgtctgcatgcatgtgtgccgtttacgctaaactgcgctcaatacatcgcacccacggtaccctccggaataggtatgtcactactcctacttacaataaggatattgaactgccccttcctttagctatcgctatacaaggtctaggtgccttccgcactgaaagcattctcgcaaaccgagttatcgctccgacctaccctgaggccacccagtatgaaggacgcgctcaagacgctttcaacatgactgagtaccagaactacatcccaactcttaaagatcttggtatcccctgtaaatcagtggaaatccatgacaagaagggaaccgcttggtggagttacaaaatccacaacgaccacggtacatcagatctcgtctgcctgcttcctcctacaaactactctccgcttgctgttgcaacccgaatgctcttcctcgctactcaggacgattctgacgaatgtactgattttatcgaatggccagaagatctcggtgattatggaacacagctgaaagaagccccacccaactccaatgttcgtgcatttcttgccttatgccatggacccctagaagaatggagcaatggacatgcttaaatatttatttatttatggtcatgtaataataacacaacccctaagttttgctgaactcttaagttctacttgattttacaatcgctaagaactaccttatatttatttatttgtgtatttctcttaaacgctgatacttttacgtatcacatcccgcacacacacacgggatggtgatgtgttatcttctagacggacttatcgtcaatgacgataatctcctactagaaggtacacagcaccctctcatatc

SEQ ID NO: 264 - DNA, 1955 bp, White clover cryptic virus 1
  source 1..1955: mol_type = other DNA
cttgtacacactttttgcaatcaaaatagaacgcaaatcttgactgctctacctcaacttaatacctactctacatggattacctaatcactgcatttaaccgaatcactcactggttcttaactccaactaatctcgaatacatcggatcctactcccttccccctgggcttcttcgcgtaaatgatgttgctgtcgcaaatcataaagctaccttagatcgctcgtttgacaagtatctctatgaacatgagataaatcttatcactaaagagtaccgccgatctcctattgatgaagactctatacttgaggacttcttctcaggtgacctaccttatttcgaaatccctttcgacgaacacgtcgaacgtggactggaatgtatggctgccgcctttcggccgcctagaccctgtcgccctgctcacatcttagatgtgaaacatggatacccttacaaatggaacgtcaatgccgaacctcccttctccactgatgaatatttcctatcacaacgtaagacattcggtgaatttatacgaatgcatgaatatgaacacattgataaagaagatttcttccgtcgtcatcccaacattgagtcccatgactttctacgcacagtcgtcccaccaaaatttggctttttgaagtcaatgatattttcgtggacccgtcgctggcatcacataatcaagtctggattccaagactctactgacctggaacaaaccggctatttctttaaccgctttatctttcccatgctactacataccaaaactgctattgtcaagaaaaacgaccctaacaagatgcgcaccatatggggcgcttccaaaccatggatcatcgctgaaacgatgttctactgggaataccttgcttggatcaaacacaatcctggtgctacaccaatgctctggggttacgaaaccttcacaggtggctggttcagactgaaccatgaactattctgtggacttatccaacgatcctttctaactttggactggtcacgctttgataaaagagcgtactttcctctgctacgtcgaatcttgtacactgtaaagactttccttacttttgaagaaggatacgtccctactcacgcagctcccactcacccacagtggagtcaagagaacatcgatagacttgaacgtctctggctgtggactctcgaaaacctttttgaggctccaatcatcttacctgacggtcggatgtacagaagacactttgctggaataccttccggcttgtttattacccaactcttagactcctggtataattatactatgctcgccaccattctgagcgcgcttcatttcgatcccttacactgtattattaaagtgcaaggtgacgattctatacttcgtttaactactctgattcctgttgaccaacacacaaactttatggatcacatcgtcagactagccgatacatatttcaattcaattgtcaatgtcaagaagtctgaagttcgcaactcccttaatggatgcgaagtactctcctaccgtaaccacaacggcctacctcatcgtgatgagatcactatgctagcccagttctaccacactaaggctagagatcccacacctgagataaccatggcacaagccataggctttgcttacgccagttgcgccaatcacaaccgcgtactctgggtactagaagatgtatataattattatcgtgacttaggctacaggcctaatcgtgctggtttgactctcacctttggagactcaccggaccttaccatgcctgaaatgcctcttgatcattttccaactaaatctgaaattcggcgttaccacaccgaaacccactatcagaacgaagcacagaacgctcggacttggcctagaacattgtttatcaatgctccaggcgagtaaaaagccgatcgctttgtttgtttctttttt

SEQ ID NO: 265 - DNA, 2012 bp, Vicia cryptic virus
  source 1..2012: mol_type = other DNA
gatcaaaagagtaacagctcttgccacgcttttcgcaatcaaaatagaacacgaatctcagctgctctacctcaactttttacttactctacatggattacctcatttccgctttcaaccgaatcactcactggttcatcactccgaccaatttcgagtacatcgggtacttctcccttccccccggccttctccgtgtcaatgacgtcgctatcgcgaatcataaatgcacacttgaacgatctttccacacctatctctttgatcacgaaatcaaaagaataatgatagatcatcgccgctcagacatcacagaagattctattttagaagacttcttcgctggtgacttcccctacttcgaagtacctttcgacgaacacgtcgaatatggtttacagtgtatggcagacgccttccgtccgccccgaccttgccgccctgctcacatcttagatgttaagcacggttacccttacaaatggaacgtcaacgcagaattcccgttctcaactgacgaatatttccttacacaacgtaagacatttggcgagttcatccgaatgcacgaatatgaacatatcgataaagacgacttcttccgtcgccatcccaacttggagtctcacgactttctgcgtaccatcgtccctcccaaatttggttacctcaaatcaaccatcttctcttggacacgccgctggcaccacatcatcaagctcggattcaccgacacaacaggactcgacaacaatggttacctctataaccgctttatctttcccatgctcctacacaccaaaactgctattgtcaagaaagacgaccccaacaagatgcgaaccatctggggcgcttccaaaccctggatcatcgctgacacgatgttctactgggaataccaagcctgggtcaaacacaaccccggctcaacacccatgctctggggcttcgaaaccttcactggaggctggttcagattgaaccaactactcttctgtgggctaattcgtcgctcattcataacattggactggtcacgtttcgataaacgagcgtactttcctttgctacgcaagatcatgtataccgtaaaatcctttctcacctttgaagaaggttacgtaccgacacacgcagctcccaaccacccacaatggaatcaagacaaaactgacaaactcgaaagactctggctctggacactcgaaaatttattcgaagctccaatcatcctgcctgacggccgaatgtacagacgtcactttgctggaataccttccggcttattcatcactcagcttttggactcatggtataactataccatgctagcaaccattctacacgctttaggcttcaacccaagtaactgcattatcaaagtgcaaggcgatgattcaatcatccgcctcaacgttctcgtaccctccgaacgccatgaccatctcatgtcccgcatcgtcgagctcgctgaatactactttaactcaattgttaatgtcaagaagtccgagatccgtaatagactaaatggctgtgaagtactttcctaccgtaatcacaacggcctaccattccgcgatgagatcgcaatgctagctcagttctaccacactaaagctagagatcccactcccgaaattacaatggctcaagccataggcttcgcttacgcaagttgcgctacacatacacgcgtactttgggtcctcgaagatatatataactattatcgcgatcaaggatacacgcccaaccgtgctggcctcaccctcacattcggtgattctccagatctcactatgcctgagatgccccttgaccactttccaactaagtccgaaatagtgcgctacctaacatgcactaattatcggaatgaagcacaaaacgcccgcacttggccgagaacattgttcatcaatgctcccgccgagtaaagccgatcgctttgtttgtttctttttaattaaaataaaaaaaaaaaaaaaaaacaaaaaaaaattgcc

SEQ ID NO: 266 - DNA, 1734 bp, unidentified
  misc_feature 1..1734: note = unclassified Partitiviridae
  source 1..1734: mol_type = other DNA
gataatgatcaaccgaaaaggttgattttcattatctagaagtaaattttctattgtaaattttcattatgaaattaacattagaagttacttctacacactaccgtgtatagcgagtttataagatttcaaattttagagttatctaattgtttactatcttacatccgttatatacaggtatggagcatcgctttcgtggaatccctcgtggtcttatagaactcgaagaaatcccgacgcgacgcttacgcgaggagcgcgttgtacatatagacgcctggagctcaaaagctatagacgccatcgtaccgctctcacttcgcatcgaactagatggctgggcccgaagttattatacacttcaagcgcacattgattcaatcatgcaatatgatcgtccaaagctgccgcaacccaccaatgctgcgtggaacaccaccacacaacacgtccgaacacagttcgcacggatggataaagtacagactctgtcttacttacaacttgaccaagttaaatgggtcagatcctccgccgctggttatggttacgttggtcgcaaaagcgataatgataactattttagagctcgaaagactgctttcactatagcagaaaagctcaaccacgatcgcgactacggtccgctagctctcgaagactctacacctgacatagcgttcacacgaacacaactatgccaaatcaaggtgaagagaaagattaggaatgtttggggagaggcgtttcattacgtcctacttgaaggtctgttcgcagatcccattattcagcactttatacggaacaagtcgttctactttattggagaagacccgctgcttgctgtcccccgcctagtcgagaagatattatcagaacaagactacgtctacatgtttgactggtctggtttcgatgcctcagtacaagaatgggaaattcgttttgccttttcgttacttgagagtatcctgatctttccctcaagtgtcgaatcttacatttggcatttcattattgagctgttcatctaccgcaagatagccgcacccaatggcaaggtataccttaaaactttaggaataccatctggtagctgtttcacgaacattattggaagtatcgttaactacgttcgcatccagtatctgttcttccgactcaccaacaacttcgtaactgttttcacacacggtgacgatagcctggtcggagtatcaactactcaatacgttcagatggacaattttgaacccatctgtgctgaacacaattggacgattaacatcgccaaatctgctgtctcgcatgaagctgaaggggtatccttcttaagccgaaaagtccgtgagcactgccatgcccgtgacgaattactctgccttcgaatgctcaaatttccagaatacgtcgtagaatctggcgcaatgtcaactttgcgtgcacattccattcaccaagacgctggaatcaattcccgttacttgtattcaatatacaaataccttttacacaggtacggaaaagctgactccctgcctctgaaccaacagaactgggaccctctagagtacgagaacctacgtgtttcttttgctactcaaaactatgaataatgtaatgatgtttatcaatacactgtttgttgtcaaccaaccgaatattttatgtatttaaatttacatagaatgtgggttaggagcctgatcactcctaacccctctgatc

SEQ ID NO: 267 - DNA, 1717 bp, unidentified
  misc_feature 1..1717: note = unclassified Partitiviridae
  source 1..1717: mol_type = other DNA
gataatgatctattagagatcattatcataggttttagttagaaattctaggttgcttgtgtgtgaaaactatagtacctgttgagtatctaaccactacccttctgttttgcaggttaggtaaaatttttaagttttaacgcgaagatttttcaccgtaccagcgcgaaactagaagatggaccacgagttcaggaagattcgtgaaggtctcatagagatcggcacagtttcccttcgtgtccaaagagacgagtttaaggtcatcattgatgaatatgccgcagaagctgttttcaagtttgttccttcaactatgctctcacaactccagggttgggccagaagtgtttactcactagatcaacatgttgatgccatacttgcgtatcgtcgtcaaaagttgcctgagccgacagatgatgtttggaaccaaaccaaacaacacaccttgcaattgttccgaagattccccaagatcacaccgatctcgtacaagtcgtttgacgaagtaaagtggatatcttcatcctccgccggttatggttacgtcggtcacaaaggtgacggtgacaattacctcaaagcgagaaggaccgcagtgacgatcgctgaaaagctcgatcacgacaggaactacgcacctgaagctattaaccagtctacacctgacgttgcttttacaagaacgcaactctctcaagtcaaagtgaagaccaaggttagaaatgtatggggtgaagcgtttcattacgttttgctggaaggcctttttgccgacccattgatcaacttcttctccaacgaagaatctttctactttataggaagaaatccgttgctcagtgtacctacattaatcgaggaaatcttcaaatccaaggactacgtatatgcgttcgattggtccgggtttgatgcctccgttcaagaatgggaaatccgtttcgcctttcaatgtctggagtcacagctgatatttccttcaaatgtcgaagcccagatctggcgtttcatcgttgagctgttcatctaccggaagattgccgccccaaacggcaccctatttctgaagaccttaggtatcccctctggtagctgttttaccaacatgattggtagcgtcgtcaactacgtccgcatccaatacatgttcaagaagttaactgatgactttgtcgaagcctacactcatggagatgatagtctagctgccgtcagtaccgctcaatacataccgctcgagaagttcggacccatatgcgaaccgtttatgtggagtatcaatactctaaagtctgaagtctcacgtgaaggtagacttactacctttctcagtcgatcgatccgcgataagcaaaactaccgtgacgaattcgtttgcctgcgtatgctcgtgtatcccgaatacgaagtggaagatggaagcatctcagccctccgtgccaaaagcatctatgtcgatgccggaatacattcccaatacttgtaccatgttttcttgtatttgaaacaaaaatatggattagccaataccctccctcacaacctgaggacctgggaccccacagaacatgaagctcttagagcttcttattctaatatcatgtaacactatttaataagttactctttctcaataaaagtttaaaatttaaattgtgctgcgtgtactattacagtagtaataagttcatgcagtacaataacaaatgtc

SEQ ID NO: 268 - protein, 487 aa, White clover cryptic virus 1
MNQDTPLANLNGPEVPSGNVPPANPPGRTNVAPPAQGAVQQPPAPAARRARNPHGPVPPAGPSRSAGAPAMLELSAAYPMYTEQRRAPNYYVPDAQLLFHTLGVCDQLMTTTDRFLRSMPSWLPIVSQLYVSVLWNVMILKVYVNTGYGAAYAHDLDVLLNHLQINECMIPGPLVPFFQSLAAVNGPFDWIGDIIPAMPSFTELWTDEFAPHAAYARQIPIPAILLDQLYRFATLAFDAQLQTNYATFEWYSNIFNQDVNTHNARLRLGPQLCGSLFTTQAQCDSARAFWNPAFANGFTRIDAANGPLMAFPQLLGFISQDGALQSNWFMHISLIMHKYAQYFNGSVPLKSISPVGIGASVIYGTPLEDTNVRDWLYPAAAAIAPFRSTRFLPRRELPATLAVRFAHADHEIEEQAEQYSILCHTNMKWYVNNATQNNHTAIEGNYIHQGEYWNFTPFRYSPPVSLKTQFAQVIASRYHQQAANRAE

SEQ ID NO: 269 - protein, 487 aa, Vicia cryptic virus
MEAHTPAADVNGPNIPSGAVSQPEIAPHNQAAPNVSGAAQAIIAPTPARKRTPHGPIPAATSSGNTSAPALLELAAAYPMYTEQRRSFNFFIPDSQMMFHSLGICDSMMNSTDRFLRSSPAWLPIVSQLYISVLWNVMIIRILSHTGYAPSFTDLLNTLTTDLQIEECMVPGPLVPFFQSLASTNGPFDWIGDITPALPGFDSLWDATNFTPHASYTRFVPIPAILLDQLYYCATYVGVNQGDLYPTFTWYRNIFTRTGANTPALLRMGPNLCGSLFTTSNQFDAARTYWRACFGTGFTRVNVTAAAFTNYLQLLGLRSQTGEPQTAWFQNVTMVMQKYAQHFNGSVPLKSIDLTGIGAVALTGTPVNNTAVRDWLYPPNANIEPFTTGRFNPRREIPAQLRIRFSHSDHELELQAEQYAIAAHTNIRWAANIATQHERTAINPAHLHQGDYWNMTPFRHTGHLGLKTQYAQLIASRYHQLAANRVD

SEQ ID NO: 270 - protein, 505 aa, unidentified
  REGION 1..505: note = unclassified Partitiviridae
MAHRTPTNAPALPQVDGINPNLPPNDPGTVAAAPNARNLHLEREQEARQDRAAAAFVPNTRFAVRRPTIRPSPAHSAPDLETALFDSAVNHPPTPVSYPSTSSYIPNFTSAFYYLNKMDSLMVQTLNWTNNCSGWVPPYSQIYISMLLYLQVMRAMKKAGVLRPNSELSHLFNEMSTIFPFESLMVPGPLVNLFENITAFRPLQTDSFGNVTPFLPAEPGWSNATFFAPNGSLVRHLPHIPALISRLRRICETASENGLNDISFSAHHHGPEFISELFGHICDNDLPEQLLLLTPGLATSYSGTLYLWRQARSQLQRSLFPEALTVNDVVPNTWTSFLCLDNDDSWFSPLAAMMNKYCQYWHGSAPLSSIPADGSAAGSMICNELNGSSIYRLAQWTAPIMDPPADEDAPPVIAEPGYYYPNPNPVLIFDARTCIEDISSAHMFSAMTFHPNLIPYGANRANFLKGQFWNCHPPSYSAPAHQVYPAVSALIAREYHSETNLSSDK

SEQ ID NO: 271 - protein, 346 aa, unidentified
  REGION 1..346: note = unclassified Partitiviridae
MASPRDSMMTTAQDAKKRKVSHILSPIRMIEPAAQSLAIIPVVPQVQMDDAEAMAELLYRERARIELDINPAYISRLESRREIGFYPPVASKNVPFTTFNIDVAEFRRCITHVLTAALKVRYYDRSNLRNAEINTEINTIVPRTVSACMCAVYAKLRSIHRTHGTLRNRYVTTPTYNKDIELPLPLAIAIQGLGAFRTESILANRVIAPTYPEATQYEGRAQDAFNMTEYQNYIPTLKDLGIPCKSVEIHDKKGTAWWSYKIHNDHGTSDLVCLLPPTNYSPLAVATRMLFLATQDDSDECTDFIEWPEDLGDYGTQLKEAPPNSNVRAFLALCHGPLEEWSNGHA

SEQ ID NO: 272 - protein, 616 aa, White clover cryptic virus 1
MDYLITAFNRITHWFLTPTNLEYIGSYSLPPGLLRVNDVAVANHKATLDRSFDKYLYEHEINLITKEYRRSPIDEDSILEDFFSGDLPYFEIPFDEHVERGLECMAAAFRPPRPCRPAHILDVKHGYPYKWNVNAEPPFSTDEYFLSQRKTFGEFIRMHEYEHIDKEDFFRRHPNIESHDFLRTVVPPKFGFLKSMIFSWTRRWHHIIKSGFQDSTDLEQTGYFFNRFIFPMLLHTKTAIVKKNDPNKMRTIWGASKPWIIAETMFYWEYLAWIKHNPGATPMLWGYETFTGGWFRLNHELFCGLIQRSFLTLDWSRFDKRAYFPLLRRILYTVKTFLTFEEGYVPTHAAPTHPQWSQENIDRLERLWLWTLENLFEAPIILPDGRMYRRHFAGIPSGLFITQLLDSWYNYTMLATILSALHFDPLHCIIKVQGDDSILRLTTLIPVDQHTNFMDHIVRLADTYFNSIVNVKKSEVRNSLNGCEVLSYRNHNGLPHRDEITMLAQFYHTKARDPTPEITMAQAIGFAYASCANHNRVLWVLEDVYNYYRDLGYRPNRAGLTLTFGDSPDLTMPEMPLDHFPTKSEIRRYHTETHYQNEAQNARTWPRTLFINAPGE

SEQ ID NO: 273 - protein, 616 aa, Vicia cryptic virus
MDYLISAFNRITHWFITPTNFEYIGYFSLPPGLLRVNDVAIANHKCTLERSFHTYLFDHEIKRIMIDHRRSDITEDSILEDFFAGDFPYFEVPFDEHVEYGLQCMADAFRPPRPCRPAHILDVKHGYPYKWNVNAEFPFSTDEYFLTQRKTFGEFIRMHEYEHIDKDDFFRRHPNLESHDFLRTIVPPKFGYLKSTIFSWTRRWHHIIKLGFTDTTGLDNNGYLYNRFIFPMLLHTKTAIVKKDDPNKMRTIWGASKPWIIADTMFYWEYQAWVKHNPGSTPMLWGFETFTGGWFRLNQLLFCGLIRRSFITLDWSRFDKRAYFPLLRKIMYTVKSFLTFEEGYVPTHAAPNHPQWNQDKTDKLERLWLWTLENLFEAPIILPDGRMYRRHFAGIPSGLFITQLLDSWYNYTMLATILHALGFNPSNCIIKVQGDDSIIRLNVLVPSERHDHLMSRIVELAEYYFNSIVNVKKSEIRNRLNGCEVLSYRNHNGLPFRDEIAMLAQFYHTKARDPTPEITMAQAIGFAYASCATHTRVLWVLEDIYNYYRDQGYTPNRAGLTLTFGDSPDLTMPEMPLDHFPTKSEIVRYLTCTNYRNEAQNARTWPRTLFINAPAE

SEQ ID NO: 274 - protein, 479 aa, unidentified
  REGION 1..479: note = unclassified Partitiviridae
MEHRFRGIPRGLIELEEIPTRRLREERVVHIDAWSSKAIDAIVPLSLRIELDGWARSYYTLQAHIDSIMQYDRPKLPQPTNAAWNTTTQHVRTQFARMDKVQTLSYLQLDQVKWVRSSAAGYGYVGRKSDNDNYFRARKTAFTIAEKLNHDRDYGPLALEDSTPDIAFTRTQLCQIKVKRKIRNVWGEAFHYVLLEGLFADPIIQHFIRNKSFYFIGEDPLLAVPRLVEKILSEQDYVYMFDWSGFDASVQEWEIRFAFSLLESILIFPSSVESYIWHFIIELFIYRKIAAPNGKVYLKTLGIPSGSCFTNIIGSIVNYVRIQYLFFRLTNNFVTVFTHGDDSLVGVSTTQYVQMDNFEPICAEHNWTINIAKSAVSHEAEGVSFLSRKVREHCHARDELLCLRMLKFPEYVVESGAMSTLRAHSIHQDAGINSRYLYSIYKYLLHRYGKADSLPLNQQNWDPLEYENLRVSFATQNYE

SEQ ID NO: 275 - protein, 477 aa, unidentified
  REGION 1..477: note = unclassified Partitiviridae
MDHEFRKIREGLIEIGTVSLRVQRDEFKVIIDEYAAEAVFKFVPSTMLSQLQGWARSVYSLDQHVDAILAYRRQKLPEPTDDVWNQTKQHTLQLFRRFPKITPISYKSFDEVKWISSSSAGYGYVGHKGDGDNYLKARRTAVTIAEKLDHDRNYAPEAINQSTPDVAFTRTQLSQVKVKTKVRNVWGEAFHYVLLEGLFADPLINFFSNEESFYFIGRNPLLSVPTLIEEIFKSKDYVYAFDWSGFDASVQEWEIRFAFQCLESQLIFPSNVEAQIWRFIVELFIYRKIAAPNGTLFLKTLGIPSGSCFTNMIGSVVNYVRIQYMFKKLTDDFVEAYTHGDDSLAAVSTAQYIPLEKFGPICEPFMWSINTLKSEVSREGRLTTFLSRSIRDKQNYRDEFVCLRMLVYPEYEVEDGSISALRAKSIYVDAGIHSQYLYHVFLYLKQKYGLANTLPHNLRTWDPTEHEALRASYSNIM

SEQ ID NO: 276 - protein, 479 aa, unidentified
  REGION 1..479: note = unclassified Partitiviridae
MEHRFRGIPRGLIELEEIPTRRLREECVIHIDAWSSQAIDAIVPLSLRNELDGWARSYYTLQAHVDSLMQYDRPKLQPPTNTAWNITTQYIRTEFARMKKVTALSYLQLDQVKWVRSSAAGYGYTGRKSDGDNYIRARKTAFTLAEKLNHNRDYGPLALEDSTPDVAFTRTQLCQIKVKRKIRNVWGEAFHYVLLEGLFADPLIQQFMRIKSFYFIGEDPLLAVPRLIEEILSEQDYIYMFDWSGFDASVQEWELRFAFGLLESILIFPSSVEHQVWQFIIELFIYRKIAAPNGKIYLKTLGIPSGSCFTNIIGSIVNYVRIQYMFFRLTREFVTAFTHGDDSLVGVPTTQYVQMENFKPICDENLWTINIAKSAISREAEGVSFLSRKVREMCHARDELICLRMLKFPEYIVETGAMSTLRAFSIHKDAGIHSRYLYQIYKFLLHRYGKADSLPLNQQNWDPIEYENLRVSYATQNYE

SEQ ID NO: 277 - protein, 616 aa, Beet cryptic virus 1
MDYLTSAFNRITHWFNVPSNLEYIGTFSLPPGILRVNEVAISNHKKTLEHSFNKYLYAHEIKLITQDYRRSDIDEESILADFFSGDVEKFEVPFDEHVETGLRCMADAFRPPRLCRPAHILDVKHGYPYKWNVNAEPPFSTDEYFLNQRKTFGEFIRMHEYEHIDKADFFRRHPNTESHDLIRTIVPPKFGYLKSMIFSWTRRWHHIIKEGFTESTGLHTTGYFYNRFIFPMLLHTKTAIVKQDDPNKMRTIWGASKPWIIAETMLYWEYIAWIKLNPSVTPMLWGYETFTGGWFRLNRDLFCGFLQRSFLTLDWSRFDKRAYFPLLRRILYTARTFLTFDEGYVPTHSAPTHPQWDHTKAIRLERLWLWTLENLFEAPIILPDGRMYRRHFAGIPSGLFITQLLDSWYNYTMLATLLSALGFDPKHCIIKVQGDDSIIRLNVLVPQDQHQNLMDNLVQLAVNYFNAVVNVKKSEFGNSLNGREVLSYRNHNGFPHRDEIMMLAQFYHTKARDPTPEITMAQAIGFAYASCANNKRVLWALKDVYDYYKDLGYTPNRAGLTLTFGDSPDLFVPEISLEHFPTETEIRRYLTSTSYLNEAQNARTWPRTLFINAPAQ

SEQ ID NO: 278 - protein, 481 aa, unidentified
  REGION 1..481: note = unclassified Partitiviridae
MRRLRNPLAGYVYTGFISDLEETNQRHTNVIRRETAVTYRDEFALQSILDLDYRQYEQHLQGWSRSYYTPEKHQEAILQYSYPDIPVQAIKMDVYNDCIAQVQNEFRSLPNVRAYSVLTQLDLVKYKSSSAAGYGYQGTKGNPGELNHARAISRAKAVLWSAIAADGEGIEHVIKTSTPDIGYTRTQLTDLTEKTKVRQVWGRAFHYILLEGLVADPFIQAVMENNTFIHAGRDPTLSVPQLLSHIAETSDWIYSLDWKQFDATVSRFEIESAFTIIKSKTIFPDRETEDAFEITKQLFIHKKVAAPDGKIYFAHKGIPSGSYYTSLVGSIVNRLRIEYLYRLILGRGPKICHTLGDDSLVGDDELIVPSSFGLIANRIGWYFNIEKTEYSRIPEMVSFLGRSYKGGLNVRDLKRCLRLLVFPEYPVPSGRISAYRAKSIAEDCGGISDLLNRVAARLKRQYGLASEEEVPIYFKRYLPFM

SEQ ID NO: 279 - protein, 472 aa, Fig cryptic virus
MEAGLIEIGNIPERHLRDEFIILVDQPAYDSVRRNAPQADMQEIDGWARSFYTVEGIMASIMQFSKPLIHEPTDPIWNDVKRETLMKIGSLFPQVQSLPFEGGFDHVPFESSSSAGYGYDGKKGEGNNFHRAKSIANAAVRKFSEDIDNQGYDYAVSHLIQQGTPDIAFTRTQLAKLPSIKVRIVFGEAFHNILIEGLSAAPLLEAFKRMDTFYFTGKDPTIYVPRILHKMSTNEGWFICLDWKAFDASVQLWEIDHAFNCIQQLLAFPTELSRLAFLFTRESFKQRKLADPNGILWMRKGGIPSGSYYTNIIGSVINYNRIEYVCKRLGLQKTSCYVQGDDSLIHITGDAKPDLTQLQMLGEQFGWTLNIPKCSLTQDSQLVTFLGRSQMHQLNIRERLKVLRLMCFPEYKVEDPKISTTRVKAIARDAGWSDPVYNKIYLQLKRLYGEVERLPPHLATFVDRFDFQDVNM

SEQ ID NO: 280 - protein, 477 aa, unidentified
  REGION 1..477: note = unclassified Partitiviridae
MALRSITGYEFHDFQSSLELLNQTHIHIVRRESGVTYHDEFALCELLVDNTRLYEQELEGWSRSYYTGEQHMKAILQYSLPNTPIQDIDVGCYQQAMTNVQERLSSLPIVRAFDVLTQLDQVSFESSSAAGYDYTGAKGPKNEGNHERAIRRAKAVLWSAIAQDGEGIEHVLRSSVPDVGYTRTQLTDLSEKTKVRGVWGRAFHYILPEGTSADPLLQAFKEGGTFYHIGQDPTVSVPYILSDTAGKCAWLYALDWSSFDATVSRFEIHAAFDLLKQRIEFPNFETEQCYEICRQLFIHKKIAAPDGKVYWAHKGIPSGSYYTSIIGSIINRLRIEYIWIKLRGHGPTICYTQGDDSLCGDDERIEPERIADIANPIGWLINPAKTATTRYPEYITFLGRTCYGGLNHRDLIRCLRLLIYPEYPVPSGAISAYRANSIAEDCGGTSSILNDIARRLTRKYGRVSHEEVPKELRVYRH

SEQ ID NO: 281 - protein, 616 aa, unidentified
  REGION 1..616: note = unclassified Partitiviridae
MDYLTTAFNRITNWFTKPANFEFVGLYSLPPGLLRVNDTAISNHKKTLEHSFSKYLYEDEIHLITKEFRRSDIDLDSILDDFFSGDVEPFEIPFDEHVETGLRCMADAFRPPRLCRPVHILDVKHHYPYKWNVNAEPPFSTDEYFLSQRKTFGEFISMHEYEHIDKADFFRRHPNRESHDLLKTTVPPKFGYLKSVIFSWTRRWHHVIKDGFKDDTGLKTTGYFYNRFIFPMLLHTKTAIVKHNDPNKMRTIWGASKPWIIAETMLYWEYIAWIKLHPSVTPMLWGYETFTGGWFRLNNQLFCGMIKHSFLTLDWSRFDKRAYFPLLRRILYQVRTFLTFDEGYVPTHAAPYHPQWDHEKALRLERLWLWTLENLFEAPIILPDGRMYRRHFAGIPSGLFITQLLDSWYNYTMLATILSSLGFNPKQCIIKVQGDDSIIRLTVLIPHENHLKFMERLTELATYYFNAIVNVKKSEVANTLNGREVLSYRNHNGFPHRDEINMLAQFYHTKARDPTPEITMAQAIGFAYASCGNHKRVLWVLNDIYNYYKDKGYTPNRAGLSLTFGDSPDLFVPEIPLDHFPTTKEIRRYLTCSSYVNEAQNARTWPRTLFLKDPAE

SEQ ID NO: 282 - protein, 478 aa, unidentified
  REGION 1..478: note = unclassified Partitiviridae
MRPSITGYDYTNFTQDLLKSDRKHPHVVRRETATTYRDDFAFKEVISLDRLAYIQRLEGWSRSYYLPEKHLEALLQYATPNVPCTALNLNVYRQAIQVVENGLRSLQPVRAFDVLTELNQISYKQSSAAGYDYIGAKGPIDGENHKRAISRAKAVLWSVVKEDGEGIDHAIETSVPDVGYTRTQLADLTEKTKVRQVWGRAFHYILLEGLVAQPFIQSIMEGPSFIHTGRDPTLSVPQSLAKVSSQCKYIYSLDWKSFDATVNRFEINTSFDIIKSKVIFPNYETEQAFEITRQLFLHKKVAAPDGYIYEAHKGIPSGSYYTSMVGSIVNRLRIEYIWRIATGHGPIHCETLGDDSLCGDDIFVPATQLADIANRIGWYFNADKTEYSTIPEGVTFLGRTSTGNLNSRDLTKCLRLLVYPEYPVTSGRISAYRARSIADDSGGLSDLLNQVAIRLERSYGIASEEEIPAYFKRYVPFM

SEQ ID NO: 283 - protein, 616 aa, unidentified
  REGION 1..616: note = unclassified Partitiviridae
MDYLTSAFNRITHWFTKPTNLEYIGDYSTPPSIIRVNEVAIANHKKTLRYAFEQYLYDHEYRMIVDEYRRSDIDQESILADFFSGDIEPFDIPFDEHVEHGLRCMANAFRPPRPCRPAHILDVKHGYPYKWNVNAEPPFSTDPYFLSQRKTFGEFIQMHEYEHIDKADFFRRHPNTESHDFLTTVVPPKFGYLKSMVFSWTRRWHHVIKTGFQDLTGLENSGYLYNRFIFPMLLHTKTAIVKKNDPNKMRTIWGAPKPWIIAETMLYWEYIAWIKLNSGKTPMLWGFETFTGGWFRLNNILFCGLIKQSFLTLDWSRFDKRAYFSLLRRILYTARTFLTFDEGYVPTHAAPTHPKWNHQKALNLERLWLWTLENLFEAPIILPDGRMYRRHFAGIPSGLFITQLLDSWYNYTMLATLLSALGFDPKYCIIKVQGDDSIIKLTTLIPRDQHENFMVQLTALATTYFNATINVKKSEIRNTLNGCEVLSYKNHNGFPHRDEITMLAQFYHTKARDPTPEITMAQAIGFAYASCGNNKRVLWILRDIYNFYKMRDVTPNRAGLTLTFGDSPDVFMPEIPLDHFPTITEIRRYLTCSEYRNEAQDARTWPRSLFISGPAE

SEQ ID NO: 284 - protein, 478 aa, Pepper cryptic virus 2
MAIYRMLNGYVFTAFGNDLEKLDQRHIHHIRREEATTYRDEFALKELMDLSPILYEQFLEGWSRSYYEGSKHLQAIIQYGIPDPDPGLIDNDIYNKAGYVVLESLGSLPRVRAFDVLTELDSVHYEQSSSAGYDYHGPKGPIQGENHIRAITRAKATLWSAIKDEGEGIEHVIRSAVPDVGYTRTQLTDLYEKTKIRGVWGRAFHYILLEGTIANPLLDVFKRGGTFYHIGENPQYSVPDILSQVSECCKYLVAIDWSNFDATVARFEINMAFDLIKTLIMFPNIETELCFEICRQLFIHKKIAAPDGNIYWSRKGIPSGSYFTSIIGSIVNRLRVEYIFRKAYDVGPKMCYTQGDDSLIGVDFRVDPDRLSEIAAPLNWKLNPAKTDVSLYPEHVTFLGRTMYGGINQRDLKRCLRLLIFPEFPVPSGEISAYRAVSIAQDAGGTSEILNSIAKRLRRQYGVAEEHAVPKHFKLYIP

SEQ ID NO: 285 - protein, 475 aa, unidentified
  REGION 1..475: note = unclassified Partitiviridae
MHNINNYEFTDFTEEVEETGRTHHHVVRREPEVTYEDRFASQVLRHKYPVLYENIISGWSRSYYSGAEHMKSIMQYATPNIKLERLHDGVYHKAIANVRESLRSLPTVRAFNVLNELDLVPFESSSSAGYDYTGVKGPMYGENHERAIRRAKATLWSAIALDGEGIDHVIRTYVPDVGYTRTQLTDLREKMKVRGVWGRAFHYILLEGVVAAPLLEAFSSADTFFHIGKDPTVSVPYLLSTIKSTAEWITAIDWQSFDATVSRFEINAAFDIIEDIVSFPNFETEQAFEISRQLFIHKMLAAPDGKIYWIHKGIPSGSYFTSTIGSLVNRLRIEYMWILKFDRGPKICYTQGDDSLIGDDELFSPMDMTFLVKPYNWLINVTKSMASKIPEAVTFLGRTSIGGLNQRDLKRCLRLLILPEYPVTSGDISAYRAKAIWYDSGLTSEVLGEIARALRRKYGLAEKSKVPKYLIPWKG

SEQ ID NO: 286 - protein, 479 aa, Pepper cryptic virus 1
MVRGTLVGYDYTQFQGDLVKSTHRHPHVVHREIATTYVDQYAYEHIETFSSLYPELILKGWSRSYYLPEKHLAAVLNYSMPNVPASQLSQSLYRQAIESAKNGFISLPRVKAFDVLTEMDQVPFKSSSSAGYNYTGRKGLIGDENHSRAISIAKAVLWSAIKDDGEGIEHVIRTSVPDVGYTRTQLTDLLEKTKVRQVWGRAFHYILLEGLVAYPFIQTVMSHKTFIHAGQDPLISVPRLLSDVALNCKWIYSLDWSQFDATVSRFEIHAAFDIIKSYVDFPNYETEQAFEITRQLFIHKKVAVPDGYIYESHKGIPSGSYYTSLVGSIINYLRINYLWRLLTGHPPQQCHTLGDDSLVGDNSYVNPQAIEEAANKLGWHFNPDKTQYSTVPEEITFLGRTYVGGLNKRDLTKCIRLLVYPEYPVESGRISAYRAKSIAQDAGGLSEVLNRIADKLRRIYGTASEEEVPIYFKRYVFGV

SEQ ID NO: 287 - protein, 616 aa, Carrot cryptic virus
MDYLTTAFNRITHWFTTPTDFEYIGNFSLPPGLLRVNDTAISNHKKTLEHSFSKYLCADEIHLITKEYRRSDIDLDSILDDFFSGDVEKFEIPFDEHVETGLRCMADAFRPPRLCRPAHILDVKHHYPYKWNVNAEPPFSTDEYFLTQRKTFGEFIRMHEYEHIDKADFFRRHPNHESHDLLQTIVPPKFGYLKSTIFSWTRRWHHVIKDGFKDSSGLQTNGYLYNRFIFPMLLHTKTAIVKHNDPNKMRTIWGASKPWIIAETMLYWEYIAWIKLNPGTTPMLWGYETFTGGWFRLNHQLYCGMIKHSFLTLDWSRFDKRAYFPLLRRILYPVRSFLTFDEGYVPTHAAPYHPQWNHEKALRLERLWLWTLENLFEAPIILPDGRMYRRHFAGIPSGLFITQLLDSWYNYTMLATILSALGFDPKQCIIKVQGDDSIIRLTVLIPQETHERFMAHLTHLATYYFNAIVNVKKSEVANTLNGREVLSYRNHNGFPHRDEINMLAQFYHTKARDTTPEITMAQAIGFAYASCGNHNRVLWVLNDIYNYYKDQGYSPNRAGLTLTFGDSPDLFVPEIPLDHFPTKKEIRRYLTASSYINEAQNARTWPRDLFINRPAE

SEQ ID NO: 288 - protein, 475 aa, Beet cryptic virus 2
MRTINNYEYTSFTEDLEETDYTHPHVVRRDPEVTYEDTFAKKELLSRYPALYENLIRGWSRSYYTGQEHLRAIMQYATPNTNFSECVQHAYTKAITKVTESLHSLPTVRAFNVLEELDLIPYESSSSAGYTYRGVKGPQHGENHMQAIKTAKAVLWSVIKDDGEGIEHVIDTYVPDVGYTRTQLTDLREKMKVRGVWGRAFHYILLEGTSAAPLLEAFANSNTFFHIGKDPTVSVPYLISYTKGQAPWLTAIDWQAFDATVSRFEINAAFDIIKSKITFPNLETEQAFEISRQLFIHKKLAAPNGKIYRIHKGIPSGSYFTSIIGSVVNRLRIEYLWNLKFNRGPKVCFTQGDDSLIGDDELYSPMDMAAFVKPLNWFINTSKSMTSKVPEAITFLGRSSLGGLNQRDLKRCLRLLILPEYPVTSGDISAFRANSIWRDSGSTSQILHEIANALRRKYGIAKEQDVPRYLRPWKA

SEQ ID NO: 289 - protein, 477 aa, unidentified
  REGION 1..477: note = unclassified Partitiviridae
MLRDIRNFQLTEFNSEFELLNDTHPHVIRREAEQILVDEFALSELNGAWPSLYDQKLKGWARSFYTLEGHMQAIHAYSNPDTPISSLNETIYRATIDSLKNELSSLPTARAFDVLTELDKIHYEQSSAAGYNYIGPKGPLYGENHERAIRSARAVLWSAIRDEDGGPAYVLRNMVPDVGYTRTQLTNLQEKTKVRGVWGRAFHYILLEGSSARPLLERFLEHDTFYHIGEDSQLSVPKLMSKLSSECRWIYAIDWSAFDATVSRFEIEAAFEIMKGLIHFPNFDSEQAFELSRLLFTHKKIAAPDGHTYWIHKGIPSGSYFTSMAGSIINRLRIGYLWRLMFNREPKLCYTQGDDSLIGDDELIQPERLAELAAPLNWNINPNKTEYSRTPEFISFLGRTSRGGFNTRELEKCLRLLIFPEHPVPSGRISAYRARSINEDSGFTSDYIQTVARRLRRRYGIAEESEVPKMFKRYVVR

SEQ ID NO: 290 - protein, 478 aa, Beet cryptic virus 3
MAYRNIREYEFTNFNEELYQIEGTHTNTIGRESEVILNDEFAKAILIDEFPVLYEQVCQGWARSFYTLEGHMQAILAYAQPDTPRETFDQTIWDQAYTAVQNELRSLPKARAFDVNTELDKVPYEQSSSAGYGYRSHKGPPGGETHMRAISRVKPTLMTAIRPDEEGPEYTILESVPDIGYTRTQLADLREKTKVRGVWGRAFHYILIEGTAARPLLENFMLGTTFMHIGSDPQLSVPRILHQMKREGSKWLYALDWSSFDSSVTRFEINCAFNLLKERIEFPNEETELAFELSRILFKHKKLAAPDGNIYMIHKGIPSGSYYTSIVGSVVNRLRIEYIWRVLFSRSPHRCYTQGDDSLIGETFLVEPETVAREAAKYGWIMNPDKTEYSTDPGYVTFLGRTAHGFMNARSLDKCLRLLMFPEYPVTSGRISAYRAESIARDCGGLSEVINLVARRLRRQYGVASEDEVPHYFKRYVA

SEQ ID NO: 291 - DNA, 1851 bp, White clover cryptic virus 1
  source 1..1851: mol_type = other DNA
atggattacctaatcactgcatttaaccgaatcactcactggttcttaactccaactaatctcgaatacatcggatcctactcccttccccctgggcttcttcgcgtaaatgatgttgctgtcgcaaatcataaagctaccttagatcgctcgtttgacaagtatctctatgaacatgagataaatcttatcactaaagagtaccgccgatctcctattgatgaagactctatacttgaggacttcttctcaggtgacctaccttatttcgaaatccctttcgacgaacacgtcgaacgtggactggaatgtatggctgccgcctttcggccgcctagaccctgtcgccctgctcacatcttagatgtgaaacatggatacccttacaaatggaacgtcaatgccgaacctcccttctccactgatgaatatttcctatcacaacgtaagacattcggtgaatttatacgaatgcatgaatatgaacacattgataaagaagatttcttccgtcgtcatcccaacattgagtcccatgactttctacgcacagtcgtcccaccaaaatttggctttttgaagtcaatgatattttcgtggacccgtcgctggcatcacataatcaagtctggattccaagactctactgacctggaacaaaccggctatttctttaaccgctttatctttcccatgctactacataccaaaactgctattgtcaagaaaaacgaccctaacaagatgcgcaccatatggggcgcttccaaaccatggatcatcgctgaaacgatgttctactgggaataccttgcttggatcaaacacaatcctggtgctacaccaatgctctggggttacgaaaccttcacaggtggctggttcagactgaaccatgaactattctgtggacttatccaacgatcctttctaactttggactggtcacgctttgataaaagagcgtactttcctctgctacgtcgaatcttgtacactgtaaagactttccttacttttgaagaaggatacgtccctactcacgcagctcccactcacccacagtggagtcaagagaacatcgatagacttgaacgtctctggctgtggactctcgaaaacctttttgaggctccaatcatcttacctgacggtcggatgtacagaagacactttgctggaataccttccggcttgtttattacccaactcttagactcctggtataattatactatgctcgccaccattctgagcgcgcttcatttcgatcccttacactgtattattaaagtgcaaggtgacgattctatacttcgtttaactactctgattcctgttgaccaacacacaaactttatggatcacatcgtcagactagccgatacatatttcaattcaattgtcaatgtcaagaagtctgaagttcgcaactcccttaatggatgcgaagtactctcctaccgtaaccacaacggcctacctcatcgtgatgagatcactatgctagcccagttctaccacactaaggctagagatcccacacctgagataaccatggcacaagccataggctttgcttacgccagttgcgccaatcacaaccgcgtactctgggtactagaagatgtatataattattatcgtgacttaggctacaggcctaatcgtgctggtttgactctcacctttggagactcaccggaccttaccatgcctgaaatgcctcttgatcattttccaactaaatctgaaattcggcgttaccacaccgaaacccactatcagaacgaagcacagaacgctcggacttggcctagaacattgtttatcaatgctccaggcgagtaa

SEQ ID NO: 292 - DNA, 1851 bp, Vicia cryptic virus
  source 1..1851: mol_type = other DNA
atggattacctcatttccgctttcaaccgaatcactcactggttcatcactccgaccaatttcgagtacatcgggtacttctcccttccccccggccttctccgtgtcaatgacgtcgctatcgcgaatcataaatgcacacttgaacgatctttccacacctatctctttgatcacgaaatcaaaagaataatgatagatcatcgccgctcagacatcacagaagattctattttagaagacttcttcgctggtgacttcccctacttcgaagtacctttcgacgaacacgtcgaatatggtttacagtgtatggcagacgccttccgtccgccccgaccttgccgccctgctcacatcttagatgttaagcacggttacccttacaaatggaacgtcaacgcagaattcccgttctcaactgacgaatatttccttacacaacgtaagacatttggcgagttcatccgaatgcacgaatatgaacatatcgataaagacgacttcttccgtcgccatcccaacttggagtctcacgactttctgcgtaccatcgtccctcccaaatttggttacctcaaatcaaccatcttctcttggacacgccgctggcaccacatcatcaagctcggattcaccgacacaacaggactcgacaacaatggttacctctataaccgctttatctttcccatgctcctacacaccaaaactgctattgtcaagaaagacgaccccaacaagatgcgaaccatctggggcgcttccaaaccctggatcatcgctgacacgatgttctactgggaataccaagcctgggtcaaacacaaccccggctcaacacccatgctctggggcttcgaaaccttcactggaggctggttcagattgaaccaactactcttctgtgggctaattcgtcgctcattcataacattggactggtcacgtttcgataaacgagcgtactttcctttgctacgcaagatcatgtataccgtaaaatcctttctcacctttgaagaaggttacgtaccgacacacgcagctcccaaccacccacaatggaatcaagacaaaactgacaaactcgaaagactctggctctggacactcgaaaatttattcgaagctccaatcatcctgcctgacggccgaatgtacagacgtcactttgctggaataccttccggcttattcatcactcagcttttggactcatggtataactataccatgctagcaaccattctacacgctttaggcttcaacccaagtaactgcattatcaaagtgcaaggcgatgattcaatcatccgcctcaacgttctcgtaccctccgaacgccatgaccatctcatgtcccgcatcgtcgagctcgctgaatactactttaactcaattgttaatgtcaagaagtccgagatccgtaatagactaaatggctgtgaagtactttcctaccgtaatcacaacggcctaccattccgcgatgagatcgcaatgctagctcagttctaccacactaaagctagagatcccactcccgaaattacaatggctcaagccataggcttcgcttacgcaagttgcgctacacatacacgcgtactttgggtcctcgaagatatatataactattatcgcgatcaaggatacacgcccaaccgtgctggcctcaccctcacattcggtgattctccagatctcactatgcctgagatgccccttgaccactttccaactaagtccgaaatagtgcgctacctaacatgcactaattatcggaatgaagcacaaaacgcccgcacttggccgagaacattgttcatcaatgctcccgccgagtaa

SEQ ID NO: 293 - DNA, 1440 bp, unidentified
  misc_feature 1..1440: note = unclassified Partitiviridae
  source 1..1440: mol_type = other DNA
atggagcatcgctttcgtggaatccctcgtggtcttatagaactcgaagaaatcccgacgcgacgcttacgcgaggagcgcgttgtacatatagacgcctggagctcaaaagctatagacgccatcgtaccgctctcacttcgcatcgaactagatggctgggcccgaagttattatacacttcaagcgcacattgattcaatcatgcaatatgatcgtccaaagctgccgcaacccaccaatgctgcgtggaacaccaccacacaacacgtccgaacacagttcgcacggatggataaagtacagactctgtcttacttacaacttgaccaagttaaatgggtcagatcctccgccgctggttatggttacgttggtcgcaaaagcgataatgataactattttagagctcgaaagactgctttcactatagcagaaaagctcaaccacgatcgcgactacggtccgctagctctcgaagactctacacctgacatagcgttcacacgaacacaactatgccaaatcaaggtgaagagaaagattaggaatgtttggggagaggcgtttcattacgtcctacttgaaggtctgttcgcagatcccattattcagcactttatacggaacaagtcgttctactttattggagaagacccgctgcttgctgtcccccgcctagtcgagaagatattatcagaacaagactacgtctacatgtttgactggtctggtttcgatgcctcagtacaagaatgggaaattcgttttgccttttcgttacttgagagtatcctgatctttccctcaagtgtcgaatcttacatttggcatttcattattgagctgttcatctaccgcaagatagccgcacccaatggcaaggtataccttaaaactttaggaataccatctggtagctgtttcacgaacattattggaagtatcgttaactacgttcgcatccagtatctgttcttccgactcaccaacaacttcgtaactgttttcacacacggtgacgatagcctggtcggagtatcaactactcaatacgttcagatggacaattttgaacccatctgtgctgaacacaattggacgattaacatcgccaaatctgctgtctcgcatgaagctgaaggggtatccttcttaagccgaaaagtccgtgagcactgccatgcccgtgacgaattactctgccttcgaatgctcaaatttccagaatacgtcgtagaatctggcgcaatgtcaactttgcgtgcacattccattcaccaagacgctggaatcaattcccgttacttgtattcaatatacaaataccttttacacaggtacggaaaagctgactccctgcctctgaaccaacagaactgggaccctctagagtacgagaacctacgtgtttcttttgctactcaaaactatgaataa

SEQ ID NO: 294 - DNA, 1434 bp, unidentified
  misc_feature 1..1434: note = unclassified Partitiviridae
  source 1..1434: mol_type = other DNA
atggaccacgagttcaggaagattcgtgaaggtctcatagagatcggcacagtttcccttcgtgtccaaagagacgagtttaaggtcatcattgatgaatatgccgcagaagctgttttcaagtttgttccttcaactatgctctcacaactccagggttgggccagaagtgtttactcactagatcaacatgttgatgccatacttgcgtatcgtcgtcaaaagttgcctgagccgacagatgatgtttggaaccaaaccaaacaacacaccttgcaattgttccgaagattccccaagatcacaccgatctcgtacaagtcgtttgacgaagtaaagtggatatcttcatcctccgccggttatggttacgtcggtcacaaaggtgacggtgacaattacctcaaagcgagaaggaccgcagtgacgatcgctgaaaagctcgatcacgacaggaactacgcacctgaagctattaaccagtctacacctgacgttgcttttacaagaacgcaactctctcaagtcaaagtgaagaccaaggttagaaatgtatggggtgaagcgtttcattacgttttgctggaaggcctttttgccgacccattgatcaacttcttctccaacgaagaatctttctactttataggaagaaatccgttgctcagtgtacctacattaatcgaggaaatcttcaaatccaaggactacgtatatgcgttcgattggtccgggtttgatgcctccgttcaagaatgggaaatccgtttcgcctttcaatgtctggagtcacagctgatatttccttcaaatgtcgaagcccagatctggcgtttcatcgttgagctgttcatctaccggaagattgccgccccaaacggcaccctatttctgaagaccttaggtatcccctctggtagctgttttaccaacatgattggtagcgtcgtcaactacgtccgcatccaatacatgttcaagaagttaactgatgactttgtcgaagcctacactcatggagatgatagtctagctgccgtcagtaccgctcaatacataccgctcgagaagttcggacccatatgcgaaccgtttatgtggagtatcaatactctaaagtctgaagtctcacgtgaaggtagacttactacctttctcagtcgatcgatccgcgataagcaaaactaccgtgacgaattcgtttgcctgcgtatgctcgtgtatcccgaatacgaagtggaagatggaagcatctcagccctccgtgccaaaagcatctatgtcgatgccggaatacattcccaatacttgtaccatgttttcttgtatttgaaacaaaaatatggattagccaataccctccctcacaacctgaggacctgggaccccacagaacatgaagctcttagagcttcttattctaatatcatgtaa

SEQ ID NO: 295 - DNA, 1440 bp, unidentified
  misc_feature 1..1440: note = unclassified Partitiviridae
  source 1..1440: mol_type = other DNA
atggagcatcgcttccgtggtattcctagaggtctcattgagctcgaagaaatcccgacgcgacgcttacgcgaggagtgcgtaatacatatagacgcctggagctcacaagcaatagacgccatagtaccgctctcactacgcaacgaactagatggctgggccagaagctactacacgcttcaagcacacgttgattcgctcatgcaatatgatcgaccaaagttgcaaccacccaccaataccgcgtggaatatcaccacacagtatattcggactgaatttgctaggatgaagaaagtgactgcattgtcttatcttcaacttgaccaagttaaatgggtcagatcctcagctgccggctacggttacacaggtcgtaaaagcgacggtgataactacattcgagctcgcaaaactgcttttactttagcagagaagctcaatcataaccgagactacggtccactagctctagaagactctacacccgatgtagctttcaccagaacacagctatgtcagattaaggtgaagagaaaaattaggaatgtttggggtgaagcgtttcattacgtcctactagaaggtctgttcgcagatccacttatccagcaattcatgcgcattaaatcattctactttattggagaagatccgctacttgctgtcccccgcctaatcgaggagattctatccgagcaagactacatctacatgtttgattggtctggctttgatgcctcagtacaagaatgggaactacgttttgccttcggcctactcgagagtatcttaatcttcccgtcgagtgtcgaacatcaagtttggcaattcattattgagctgttcatctaccgaaagattgccgccccaaatggcaagatatacctaaagactttaggcataccatccggtagttgttttaccaacatcattggaagcatcgtcaactacgtccgcattcagtatatgtttttccgactcacaagagagttcgtaaccgcgttcacacacggagacgacagcctcgtcggagtacccactactcaatacgttcagatggaaaacttcaaacccatctgcgatgagaacctttggacgattaacatcgccaagtcagcaatctcacgcgaagctgaaggtgtatccttcttaagcaggaaagtccgcgaaatgtgccatgcacgtgacgaacttatctgtcttcgaatgctcaaatttccagaatacatcgtcgagacaggcgcaatgtcaaccttgcgagccttctcaattcataaagacgctggaatccattcccgttacttgtatcaaatctacaaattcctacttcataggtacggaaaggctgactcccttccgcttaaccagcagaactgggaccccatcgagtacgagaacctacgtgtttcttacgctactcagaattatgaataa

SEQ ID NO: 296 - DNA, 1851 bp, Beet cryptic virus 1
  source 1..1851: mol_type = other DNA
atggattaccttacctccgcattcaacagaatcacacactggttcaacgtcccttccaacttggaatacatcggaaccttttcgctcccccctgggattctacgtgtcaacgaagttgctatttctaatcacaagaaaacgttggaacactctttcaacaagtacctttatgcacatgaaatcaaattaatcacccaagactatcgccgctctgacatagatgaagaatctatactagccgatttcttttccggtgacgtcgaaaagttcgaagtaccctttgacgaacacgtcgaaaccggtctacgctgcatggcagacgctttccgtccgcctagactttgccgccctgctcacatcttagatgtcaagcacggatatccttacaagtggaacgtcaacgctgaacccccgttctccactgacgaatacttcctcaaccaacgtaagacatttggcgaattcatacgcatgcacgaatacgaacacatagataaagctgactttttccgccgtcacccaaacactgaatcacacgatttgatcaggacaattgttccaccaaaattcggttacttaaaatcgatgatattttcttggacacgtcgctggcatcacatcataaaagaaggattcactgaatcaactggattacacaccaccggatacttctacaaccgttttatcttccccatgctactacacaccaaaactgctattgttaaacaggacgaccctaacaagatgcgcaccatatggggcgcctctaaaccatggatcatcgccgaaacgatgctctactgggaatacattgcttggattaaactcaaccctagcgtcactccgatgctttggggttacgaaaccttcacaggtggctggttcagactgaaccgagacctattttgcggtttcctccaacgctctttcctcaccttggattggtcacgtttcgacaagcgtgcctacttccccctcttgcgccgcattttatacactgcccgcaccttcctcactttcgacgaaggatacgtccctacacattcagcacctacccatcctcaatgggaccacactaaagcaatcagacttgaacgactatggctatggaccttggaaaaccttttcgaggcacctatcatcctacctgacggtagaatgtacagacgccacttcgccggaataccttccggactgttcatcacccaattgctcgactcatggtacaactataccatgttggccactctcctgagtgcacttggcttcgaccccaaacactgcataattaaagtacaaggcgatgattcaatcattcgacttaacgtactagtaccacaagatcagcaccaaaatttaatggacaacctcgtccaacttgctgtcaactacttcaatgcagtcgttaatgtcaagaagtccgaattcggcaactcactcaatggtcgtgaagtcctatcctaccgcaatcacaatgggttcccccaccgtgacgagatcatgatgttggcccagttttaccacactaaagctagggacccaacacctgagatcaccatggcacaagccataggcttcgcctatgctagttgcgctaacaataaacgtgtcctctgggcacttaaggatgtttatgactactacaaagacttgggatacacccccaaccgagcaggcctaaccctgacattcggtgactcccctgatctttttgttcccgaaatctctcttgaacatttccccaccgagactgagatacgtcgttatttaacgagtacctcctatctcaatgaagcacagaacgctcgcacctggccgagaacgttgttcatcaacgctcccgctcagtaa

SEQ ID NO: 297 - DNA, 1446 bp, unidentified
  misc_feature 1..1446: note = unclassified Partitiviridae
  source 1..1446: mol_type = other DNA
atgagaagattgcgcaaccctttagctggttacgtttacacaggattcatctcggatcttgaagagacaaatcagcgacacacaaacgttattcgacgggaaaccgccgtaacgtacagggatgagtttgcattgcaatccatcctagatctggactacaggcagtacgagcaacatctacaaggttggtcgcgtagttattacacccctgagaagcatcaggaagcaattttacaatactcctatccagacataccagttcaagccattaaaatggacgtatacaacgattgtatcgcccaggttcagaacgaatttcgtagccttccaaacgtgagggcctatagcgtattaacccaattagatttggttaaatacaagtcatcatcggcggccggttacggataccaaggtactaaaggaaatccaggtgagttaaatcacgccagggctatttcccgagccaaagcggttttatggtcagccatcgcagcagacggtgaaggcatcgaacatgtcatcaagacctcaacgccagacataggctatacacgtacccagcttacagatctcacagagaagacgaaagtcagacaagtatggggtagagcttttcattacatcctgttagaaggattggtcgcagacccattcattcaagcagtaatggagaacaatacattcatccatgctggacgagatccaacactaagcgtgccacagttattatcgcacatcgcggaaacaagtgactggatatattcattagattggaaacaatttgatgcaacagttagcagattcgaaattgagtcagcttttactattatcaagtccaagacgatcttcccagacagagaaacggaagatgcttttgagataaccaaacagttattcattcataagaaagtcgccgctccagacggtaaaatttacttcgcgcacaaaggaattccatcaggaagttattacacttcgctagtcggatctattgttaatagactcaggattgagtatctatacagactaattttaggacgaggtcccaagatatgtcatacattaggagacgattcactagtaggtgatgacgagttgatagtcccatcctcattcggccttatcgccaatcgtatcgggtggtacttcaatattgaaaagacggagtactcgagaataccagagatggtttcatttctaggtcgatcctacaaaggaggtctcaacgtacgagatcttaaacgatgtttaagattactagtctttcctgaataccctgtcccatcaggaagaatttcagcatacagagctaaatcgatagcagaagattgcggaggcatcagcgatctactaaatcgagtagcagcaagattaaagcgtcagtacggactcgcttctgaagaagaagttccaatctattttaagcgttatttaccttttatgtaa

SEQ ID NO: 298 - DNA, 1419 bp, Fig cryptic virus
  source 1..1419: mol_type = other DNA
atggaagcaggtcttatagagattggaaacattccagaacgacacttacgtgacgaattcataatactggtcgaccaaccagcatatgactcagtgagacgaaatgctccacaagctgacatgcaagaaattgatggatgggctagatccttttacacagtagaaggcattatggcatctattatgcaattcagcaaaccactcattcatgaaccaaccgatccaatatggaacgacgttaagagagaaacactaatgaagataggtagtttgttcccacaagttcaatcattaccatttgaaggaggttttgaccacgtcccatttgaatcgagctcatcagccggctacggctatgacggtaagaagggtgaaggtaacaactttcatcgtgcgaaaagcattgcaaatgctgcagtacgcaagtttagcgaggatattgacaaccaaggttacgactacgcagtcagccatctcatacaacaaggtacgccagatattgctttcacgcgaacacagctagcgaagctaccatcgattaaagttcggattgtctttggagaggcatttcacaacattttaattgagggactatcagctgcgccactcttagaggcctttaagagaatggacaccttttactttacagggaaggacccaacaatttacgttccaagaatcttacacaagatgagcacaaacgaaggatggtttatctgccttgactggaaagcgtttgacgcttcagttcaattatgggagattgatcacgcattcaattgcattcagcaattgcttgcttttccgacggaattatcacgactagcgttcctatttactagggagtcattcaaacaacgcaagctcgcagacccaaatgggatactgtggatgcgcaagggtggcattccttcagggagctattacactaacatcattggaagtgtaattaattacaatagaattgagtacgtgtgtaaaaggttaggtttacaaaagacaagctgttacgtgcagggagatgattctcttatccacattactggtgatgcaaaaccagatttaacacagcttcagatgcttggagagcagttcggatggacgctcaatattccaaagtgcagtcttacgcaagacagccaactggttactttcttaggaaggagtcagatgcatcaattaaacattagagagagacttaaagtactccgactgatgtgttttcccgagtacaaggttgaagaccctaaaatctcaaccactagggtaaaagcaatcgctcgagatgctggatggagtgatcctgtttataacaagatttacttacagctgaagcgcttgtatggtgaggtcgaaagacttccaccgcatttagcaacatttgttgaccgttttgattttcaagatgtaaacatgtga

SEQ ID NO: 299 - DNA, 1434 bp, unidentified
  misc_feature 1..1434: note = unclassified Partitiviridae
  source 1..1434: mol_type = other DNA
atggcgttgagatcaattacagggtatgagttccatgattttcaatctagtttagaattgttaaatcaaactcacatccatatagtcaggcgggagtctggcgttacctaccatgacgagttcgcactttgtgaactattggtagataatacaagattgtatgagcaagagctagaaggctggagtagaagttactacaccggtgaacaacacatgaaagctatattgcagtacagtttaccaaacacaccaattcaagacattgatgtcggttgctatcagcaggcaatgaccaatgttcaagaacggttaagtagccttccaatcgtgagggcattcgacgtattgacccaattggatcaagtaagtttcgagtcgtcttcggcggcaggttacgactatacaggtgcgaaaggacccaagaatgaaggaaatcatgaaagggcaattcgacgagcgaaagcagtgttatggtcagcaatcgcacaagatggcgaagggatcgaacacgtactacggtcatcagtaccggacgttggatacacacgcacgcaactaaccgatttaagtgaaaagacgaaagtaagaggtgtatggggcagagcattccattacatactcccagagggcacatcagcagatccgctgttacaggctttcaaagaaggaggaactttctatcacataggacaagatcctacggttagcgttccatatatactttccgacacagcaggaaaatgcgcatggttatatgcattagactggagttcttttgatgctacagtcagcagatttgaaattcatgcagcgtttgatcttctcaagcagagaatcgagtttcctaacttcgaaacagagcaatgttacgaaatttgcaggcaactatttatacacaagaagatcgctgcaccagacggaaaagtctattgggcacacaaagggataccatccggtagctattatacatccataatcggctcaatcatcaacagattgcggatcgaatacatatggataaagcttagagggcacggaccgaccatttgttacacacaaggtgatgattcattatgtggtgacgatgaacggatcgaaccagagcgaatagctgatatcgccaatccaattggttggttgattaatccggcaaagacggcaacaacaaggtacccggaatacataacattcctaggaaggacatgttacggaggtttaaaccatcgagatcttatcagatgtctaaggttactaatctatccggaatatccagttccgtcaggcgctatctcagcgtatcgcgctaattcaattgctgaagactgcggaggcacgtcttcgatattgaatgatatcgcgcgacgattaacacgaaagtacggtcgtgtatctcatgaagaggtaccgaaagagctccgagtgtacagacactag

SEQ ID NO: 300 - DNA, 1851 bp, unidentified
  misc_feature 1..1851: note = unclassified Partitiviridae
  source 1..1851: mol_type = other DNA
atggattacctcacaaccgcattcaaccggattaccaactggtttaccaaaccagctaacttcgaattcgtaggactctactccctacccccaggccttctgcgtgtcaatgacactgcaatttctaaccataagaagacactggaacattctttcagcaaatatctttatgaagatgagattcatctcatcaccaaagaattccgccgatctgatattgatctagattcaattcttgacgatttcttctctggcgatgttgaaccattcgaaataccattcgacgaacacgtcgaaaccggtctacgctgcatggcagacgctttccgtccgccaagactttgccgtcctgttcacatccttgatgtgaaacatcactatccttacaaatggaacgtcaatgctgaaccccccttctccactgatgaatacttcctatcacaacgtaagacattcggcgaattcataagcatgcacgaatacgaacacattgataaagccgactttttccgtcgccatcctaatcgcgaatcccacgaccttctcaaaaccaccgtccctcctaaatttggatacctgaaatcagttatcttttcttggacccgccgctggcatcacgtcattaaagatggattcaaagatgacactggcctcaaaacaaccggatacttctataatcggtttatcttccccatgctcctacacactaaaactgcaattgtcaaacacaacgatcccaacaagatgcgcaccatttggggcgcttccaaaccttggatcattgctgagactatgctttattgggaatatatagcttggatcaaactacaccctagcgtcacaccgatgctttggggttacgaaaccttcacaggtggatggttcagactaaacaaccaactcttttgtggtatgatcaaacattcattcctcactctagactggtcacgcttcgacaaacgagcgtactttccactattacgcagaattctctaccaagtacgaactttcctcacatttgatgaaggttacgtgccgacacatgcagctccgtaccacccacaatgggatcacgaaaaagcactgagacttgaacgtctttggctctggacactcgaaaacctcttcgaagcaccgatcatcttacctgacggtcgcatgtaccgacgccacttcgctggaataccttctggcctgttcatcactcaattgctcgattcctggtacaactatactatgctcgctaccattctaagctctctgggcttcaatcccaaacaatgcatcatcaaagtacaaggcgatgactcaatcattcgcttaactgttctcatacctcatgaaaatcatctgaaattcatggaacgtttgactgaactagctacttattatttcaatgccattgtcaatgtcaagaagtctgaagttgcgaacacactaaatggacgcgaagtactctcttaccgcaaccacaacggtttccctcaccgtgacgagatcaacatgcttgctcagttctatcacactaaagcaagagatccgacacccgaaatcactatggctcaagcaataggttttgcctatgctagctgtggaaaccacaaacgcgtactttgggtactgaacgacatatacaactactacaaggacaaaggctacacacctaaccgtgcaggtctgtctctaacatttggagactcaccagatctgtttgtgcctgaaatccctcttgatcatttccctacaactaaggagatacgtcgctacctgacgtgttcttcttatgttaatgaagcacagaacgctcggacttggcctaggaccttatttcttaaggatcctgctgagtaa

SEQ ID NO: 301 - DNA, 1437 bp, unidentified
  misc_feature 1..1437: note = unclassified Partitiviridae
  source 1..1437: mol_type = other DNA
atgagacccagcattacaggttatgattacacaaatttcacacaggatttgcttaaatctgaccgtaagcacccacacgtcgtacgtcgcgagactgcgactacttacagagacgactttgctttcaaggaagtaatttctctcgataggttagcttacatacagagattggaaggatggtcacgaagttactatctaccagagaagcatttagaggctttactccagtatgctacacctaatgtcccatgcactgctttgaaccttaacgtttatcgtcaagctattcaagtagtagagaatgggcttcgtagccttcaaccagtgagggcctttgatgttctaactgaactcaatcaaatttcatacaagcagtcctcagctgcaggttacgattacatcggtgcgaaaggacctattgatggtgaaaaccacaaaagggctatatcccgtgctaaagctgtattgtggtctgtcgttaaagaagacggcgaaggcatagatcatgctattgagacctcagtaccagacgtcgggtacactcgcacacagttggcagatttgaccgaaaagacaaaggttagacaagtttggggccgtgcatttcattacatccttttagaaggacttgtagcacaaccatttattcaatcaataatggaaggaccctcgtttatccacaccggtcgagatccaacactcagcgttccgcaatcattggctaaagttagctcgcaatgcaaatacatctattcattagattggaagtcgtttgacgcaaccgttaatcggttcgaaatcaacacatcttttgacatcattaaatcaaaggtcatctttcctaactatgaaactgaacaagcatttgagataaccagacagctctttcttcacaagaaggtagccgcacccgatggttatatttacgaagcgcacaaaggaattccatcaggaagttactacacctcaatggtcggatcaatcgtgaatcgactaaggatcgagtacatctggagaatcgcaacaggacatggtccgatacactgcgagactttaggagacgactctctctgcggagatgatatcttcgttccagctacacagttggccgatatcgcaaaccgcataggttggtacttcaatgccgataagacggaatactcaactataccagaaggcgtcacctttctaggacgaacgtcaaccggaaacttaaactcaagagatttaaccaaatgtcttcgactattagtttaccccgagtatcctgttacatcaggccgaatttccgcctatagagctaggtcaatagctgacgactcaggaggtctcagcgatttgcttaaccaagttgctattaggttagagcgtagttatggcattgcttcggaagaagaaattccagcttattttaaacgttatgtaccatttatgtaa

SEQ ID NO: 302 - DNA, 1851 bp, unidentified
  misc_feature 1..1851: note = unclassified Partitiviridae
  source 1..1851: mol_type = other DNA
atggattacctcacttccgcatttaaccgaatcactcattggttcaccaaacccaccaatttagaatacattggagactactctacacctcctagtatcatccgcgtcaatgaagttgctattgctaaccacaagaaaacacttcgctacgcctttgaacagtatctgtacgatcacgaataccgtatgatcgttgatgaatatcgtcgctcagacatagaccaagagtctatcctcgccgactttttctctggtgatattgaaccctttgatatacctttcgacgaacacgtcgaacatggactacgctgtatggccaacgccttcagacctcctagaccttgccgccctgctcacatcctagatgtcaagcacggatacccttacaaatggaacgtcaacgcagaaccacccttctcaactgacccttacttcctttctcaacgtaagacctttggcgagttcattcaaatgcacgaatatgaacacatcgataaagctgacttctttcgaagacatcccaacaccgaatcacacgacttcctcactacagtggtccctccaaaatttggatacctcaagtcgatggttttctcctggacccgaagatggcaccacgtcattaaaaccggattccaagacttaactggactcgaaaactctggatatctctacaatcgttttatctttcccatgctcctacacaccaaaactgctattgtcaaaaagaacgatcctaacaagatgcgcaccatctggggcgcccctaaaccttggatcatcgctgagacaatgctatattgggaatacatagcctggatcaaactcaactccggtaagacaccgatgctttggggatttgaaaccttcacaggtggatggtttcgtttgaacaacattttgttctgcggactcatcaaacaatccttcttaacattagactggtctcgcttcgacaagcgtgcctatttcagtcttctccgacggatcctttacacagctagaaccttcctcactttcgacgaaggctatgtcccaacacatgcagcaccgacccaccccaagtggaaccaccaaaaagcactcaaccttgagcgcctttggctctggacactcgagaacctctttgaggcacccatcatcctacctgatggccgaatgtacagacggcacttcgccggaattccttcaggtctatttatcacacaactattagactcttggtacaattacacgatgctagccacactcctaagcgcactgggcttcgaccctaaatattgtattattaaagtgcaaggcgacgactcaattatcaaattgacgaccttaattccacgtgaccaacacgagaacttcatggtccaactcaccgccctcgcaaccacgtactttaatgccaccatcaatgtcaagaagtcggaaatcagaaacacgctcaacggatgtgaagtcttatcatacaagaaccacaacggtttcccccaccgtgacgagatcactatgcttgctcaattctaccacaccaaagcacgtgacccaacgcctgaaattaccatggctcaagcaataggctttgcctatgccagctgcggcaacaacaaacgcgtactgtggattcttagagacatttacaacttttacaagatgcgtgacgtcacccctaatcgtgcaggtttaaccctcactttcggagactcaccagacgttttcatgcctgaaatccctcttgatcattttcctacaataactgaaattcggagatacctaacatgctctgaatatcgcaacgaagcacaagacgctagaacttggccgagatcactattcattagtggtcctgccgaataa

SEQ ID NO: 303 - DNA, 1437 bp, Pepper cryptic virus 2
  source 1..1437: mol_type = other DNA
atggcaatttatcgcatgttgaatggatatgttttcaccgcatttggtaatgatcttgagaaattggatcaaagacatatacatcacatcagacgagaagaagcgacaacatatcgcgacgagttcgctctaaaggaactcatggatttaagccctattttgtatgaacaattcttagaaggatggtcacggagttattacgaaggatcaaaacacttacaagcaattatacaatatggcatcccagacccagatccaggcttaatagataatgatatctataacaaggcaggatatgtggtattagaaagtttaggtagccttccacgtgtgagggcatttgacgtactcactgagttagactcagtacactatgagcaatcttcatccgctggctatgactaccatggaccaaaaggacccatacaaggcgaaaatcacataagggcaattaccagagccaaagcaacattgtggtcagcaattaaagatgaaggcgaaggaatagaacatgtcatcaggagcgcagttccagatgtcggttacacccgcacacaattaacggatctatacgagaagaccaaaatcagaggagtttggggtagagcgtttcattacatcctacttgaaggaacaatagctaatccattgctagatgttttcaagagaggcggaacattttatcatatcggagagaatccacaatatagcgtaccagatatcttatcacaagtatcagaatgctgtaaatatctagtagcaattgactggagcaatttcgatgctacagtagctagatttgaaatcaatatggcatttgatttaataaagacgctaattatgtttccaaacatcgagacggagttatgtttcgagatatgcagacaactcttcattcataagaagatagctgcaccggacggaaatatatactggtcgcgcaaaggaatcccatcaggcagttactttacatccatcataggatctattgtaaacagattaagagtagagtatatctttagaaaggcatacgatgtcggaccaaagatgtgttatacacagggagatgattcactaatcggagtagactttcgagtagacccagacagattaagtgagatagcagcaccattgaattggaagcttaatcctgctaaaacagacgtttcgttgtacccggagcatgtcactttcttaggaaggactatgtacggaggaataaatcaaagagatcttaaaagatgtctgaggttattaatattccctgaatttccggttccatcgggagagatttcagcatacagagcagtatcaatcgcgcaagatgccggaggcacaagtgagatactgaacagtatagcaaagagacttcgaagacagtatggagtcgcagaggagcacgcagttccaaagcatttcaaactttacattccataa

SEQ ID NO: 304 - DNA, 1428 bp, unidentified
  misc_feature 1..1428: note = unclassified Partitiviridae
  source 1..1428: mol_type = other DNA
atgcacaacatcaacaattatgagttcacagactttactgaagaagtagaagaaactggcagaacccatcatcacgtagttaggcgagagcctgaggttacatatgaagatagatttgcttcgcaagtcttaaggcataagtatccagtcttgtatgaaaacatcatttcaggatggtctagaagttactactctggagcagaacacatgaaatcaatcatgcaatacgctacaccaaatattaaactagaacgtttacacgacggcgtataccataaagccatcgccaacgttcgagaaagtttacgtagccttccaactgtgagggcattcaatgtattgaatgagttagacctcgttccattcgaatcgtcatcatcggcgggttacgactacacaggcgtgaaaggtccgatgtacggtgagaaccatgaacgggcaattcgacgagcgaaagcgacactttggtcagccatagcattagacggcgaaggcatagaccatgtcatcaggacatacgtcccagacgtcggttacacacgcactcagctaacagatcttcgagagaagatgaaagtcagaggagtctggggaagagcattccattatatacttctagaaggagtagtggcagcccctttacttgaagccttttcaagtgcagacacattctttcacattggcaaagacccaacagttagcgttccttatttgctttcaacaattaagtcaacagcagaatggataacagcaatagactggcaatcatttgatgcaacagtaagccgttttgaaattaacgcagctttcgacatcattgaagacatagtcagttttccaaactttgaaacagagcaagcattcgagatttcaaggcaactattcatacacaaaatgctggcggcacctgacggcaaaatatactggatacataaaggtatcccatccggaagttatttcacatcaacgatcggatcattagtcaacagactacgcattgaatacatgtggattctcaaattcgatagaggtccaaagatatgttacactcaaggcgatgattcactgatcggtgacgatgaactcttcagtccaatggacatgacatttctggtcaagccatacaactggttaatcaacgtaaccaagtccatggcatccaagattccagaagcagttacgttccttggaaggacaagcataggcggcctaaatcagagagatctgaagcgctgcttacgactactgatattaccagagtaccctgttacatctggagacatatcagcatacagagctaaggcgatctggtacgactcggggttaacgagtgaagtgctaggcgaaatagctcgcgctcttcggcgtaagtatggactagcagagaaatccaaagttccaaagtatttaataccttggaaaggatga

SEQ ID NO: 305 - DNA, 1440 bp, Pepper cryptic virus 1
  source 1..1440: mol_type = other DNA
atggtaagaggcacacttgttggttatgactatacccaatttcagggtgatttagttaaatcaactcaccgacaccctcatgttgttcaccgcgaaatcgcaacaacatatgtcgatcagtatgcttatgagcatattgaaacattcagtagtttatatcctgaacttatccttaaaggatggtctaggagttactatctgcccgagaagcatctcgctgcagtgttaaactacagtatgccaaatgttcctgcatctcagctcagtcagtcattatacagacaagcgattgaatcagctaagaatggatttattagccttccccgagtgaaggctttcgatgtacttaccgaaatggaccaagtaccttttaagtcgtcttcatcggcgggttataattacacaggcaggaaaggccttattggtgatgaaaatcactcaagggcaatttcgatagctaaagctgtattatggtcagccatcaaagatgatggagaaggcattgaacatgtcattcggacttcagtaccagacgttggctacactcgaactcagttaacggacttgctagaaaagacgaaagtcagacaagtttggggcagagcattccactatatcctacttgaaggattggttgcatacccgtttatacaaacagtcatgtcacataaaacgtttattcacgcaggacaagacccgttaattagtgttcctagattattatcagacgttgcattgaattgcaaatggatttactcacttgattggagtcaattcgacgctacagttagtcgattcgaaatccacgccgcttttgacataattaagtcttacgtagactttccaaattatgaaacagagcaggctttcgagatcactcgacaactattcatacataagaaagttgcagtacctgacggttacatctacgaatcacacaaaggaattccatctggaagttactacacttcacttgttggatcgattatcaattatttgagaatcaactacctctggagactcctcaccggacacccaccacaacagtgtcacactttaggagatgactcattggttggagataactcatatgttaatccacaagcgattgaagaagcagcaaacaagctaggctggcacttcaacccagataagactcaatactctacagtacctgaagagatcacatttctcggacgaacttacgtaggcggattaaataagagagatctaacgaaatgtattagattactcgtatatcccgaatacccagtagagtcaggtcgaatttcagcttatagagctaagtctatcgcacaagatgctggcggtctcagcgaagtgctaaataggatagcagataagcttagacgtatttacggtacggcttcagaagaagaggtaccaatttattttaaacgctatgtatttggagtttaa

SEQ ID NO: 306 - DNA, 1851 bp, Carrot cryptic virus
  source 1..1851: mol_type = other DNA
atggattacctcacaaccgcattcaaccgaatcacacactggttcacaacacctaccgatttcgaatatattgggaacttctccctacctccaggcttgctccgtgtcaatgacactgcaatttctaatcataagaaaactctggaacattctttcagcaaatacctctgtgctgatgagattcatctcatcactaaagaataccgtcgatctgatatcgatcttgattcaattcttgacgacttcttctctggcgatgttgaaaaatttgaaattccattcgacgaacacgtcgaaacaggtttacgctgcatggctgacgccttccgtccgccaagactctgccgcccagctcacatccttgatgtgaagcatcactacccttacaagtggaacgtcaatgcagaaccacccttctcaactgacgaatacttcctcacacaacgtaagaccttcggtgagtttatacgcatgcatgaatacgaacacattgataaagccgacttctttcgtcgccaccctaaccatgaatcccatgacctccttcaaactatcgtacctccaaaatttggctatctaaagtcaactattttctcttggacccgtcgatggcaccacgtcattaaagatggatttaaggatagctccggtctacaaacgaatggatacctatacaatcggtttatctttcccatgctcctacacaccaaaactgcaattgtcaaacacaatgatcccaacaagatgcgaaccatatggggcgcttctaaaccttggattattgctgagactatgctctactgggaatatatagcttggatcaaactcaaccctggcaccacacctatgctttggggttacgaaaccttcaccggtggatggttcagactgaaccaccaactatactgcggaatgatcaaacattccttcctcaccctggattggtcacgcttcgacaaacgagcgtactttccactcctacgtagaatactctatcctgtacgatccttcctcacatttgacgaaggttatgtaccgacacatgcagcaccgtaccacccacaatggaatcacgaaaaagctttgcgacttgaacgtctctggctctggacacttgaaaacttattcgaggcacccatcatactacctgatggacgcatgtaccgacgccactttgccggaataccatccggattgttcataactcaactgctagattcctggtacaattacactatgctcgctaccattctaagcgctctaggcttcgaccctaaacaatgcatcattaaagtacaaggcgatgactcaataatccgtctgactgttctaattccacaagaaacccacgaacgtttcatggctcatctgactcaccttgccacgtactacttcaatgctattgtcaatgtcaagaagtccgaagtagccaatacacttaatggtcgtgaagtactctcttatcgcaatcacaacggtttccctcaccgtgatgagatcaacatgcttgctcaattctatcacactaaagcaagagataccacacctgagataaccatggctcaagcaataggtttcgcctatgctagctgtggcaaccacaaccgcgtattatgggtacttaatgacatatacaactactacaaggaccaaggatattcacctaatcgtgctggcctgactctcacctttggagactcacctgacctttttgttcctgaaatcccccttgaccattttcctacaaagaaggagatccgtcgttacctgacggcttcttcctacattaacgaagctcaaaacgctcggacttggcccagagacctgtttattaacaggcctgccgagtaa

SEQ ID NO: 307 - DNA, 1428 bp, Beet cryptic virus 2
  source 1..1428: mol_type = other DNA
atgaggacaatcaacaactacgagtatacaagtttcaccgaagatctcgaggagacagattatactcacccacacgtggtcaggagagatcctgaagttacctatgaagacaccttcgcaaagaaggagttgttaagcaggtatccagcactgtatgaaaatttaatccgtggatggtcaagaagctattacactggacaagagcacttgcgtgcgatcatgcagtatgccacaccaaatactaactttagtgaatgtgtccaacacgcttataccaaagcgatcactaaagtgacagaaagtttacatagccttccaactgtgagggcattcaatgttctggaagagttagatctcattccatatgaatcgtcatcatcggcgggctatacgtatagaggcgttaaaggtccacaacatggtgaaaatcatatgcaagcgatcaaaacagccaaggcagtactttggtccgtcataaaagacgacggagaaggtatcgaacacgtcattgacacttatgtgccagacgtaggctatacgagaactcagcttactgatcttcgagagaagatgaaggttagaggagtatggggaagagcgtttcattacattcttctagaaggaacttcagcggcaccgctacttgaggccttcgcaaactcaaatacattcttccatattggaaaagacccaacagttagcgtaccatatctaatttcgtacaccaaaggacaagctccatggttaacggcaattgactggcaagcatttgatgctacagttagcagattcgaaataaacgcagcctttgatataatcaagagcaagattacgtttccaaacctggaaacagagcaagcgttcgaaatctcaagacaactgttcatccataagaagctggcagcgccaaacggaaagatatacaggatacacaaaggtatcccatcagggagttatttcacatcaattatcggatcagtcgtgaatagattgagaattgaatatttatggaacctcaaattcaacagaggaccaaaagtatgtttcactcaaggcgacgactcgctaataggagatgacgaactgtacagcccaatggacatggcggcatttgttaaaccactgaactggtttataaacacatcaaagtcaatgacttcgaaagtgccagaagccataacgttcttgggacggtcaagtttaggcggcttaaatcaacgtgatttaaagcgttgcctccgtttactaatattaccagagtacccggttacatctggcgatatatcagcattcagggctaactcaatctggagagattctggttcaacaagccaaatccttcatgagatagcaaacgcacttcgacgaaagtatggaattgctaaagagcaggacgtaccaaggtatctcagaccctggaaagcctaa

SEQ ID NO: 308 - DNA, 1434 bp, unidentified
  misc_feature 1..1434: note = unclassified Partitiviridae
  source 1..1434: mol_type = other DNA
atgttgagagatattagaaactttcaattaaccgaattcaattcagagttcgagttattgaatgacacacatccccatgtcattcggcgagaagctgaacagattttggtggatgagttcgctttaagcgaactaaacggtgcatggccatcgctatatgaccagaaactcaaaggttgggctagaagcttttataccttagagggtcatatgcaagcgatacatgcatattctaatccagatacacctatctcttctttaaacgagaccatctatcgagctacgattgatagtctaaagaatgagttaagtagccttccaactgcgagggcttttgatgtcttgactgagctagacaagatacactacgaacagtcctccgctgccggctacaattatattggacctaaaggaccactatatggtgagaatcatgaacgcgcaatccgaagtgcccgcgccgttctatggtcagcgattagagacgaagacggtggtccagcttatgtcttaaggaacatggttccagacgttggatacactcgcactcaattgactaatcttcaagagaagactaaagtccgtggagtatggggtagagcgtttcattacattctactagaaggttcttccgcaagaccacttctagagaggttcttagaacacgacaccttttaccacattggagaagattcgcaattgagcgtaccaaaacttatgtccaaattaagttctgaatgtcgatggatttacgcgattgactggtcagcatttgatgctacagtgagccgatttgaaatcgaagcagcttttgaaattatgaaaggattaatacactttccgaacttcgacagcgaacaagcattcgagctaagtaggttgttattcactcataagaagatagcagctccggacggacacacgtattggatacataaaggaattccttcagggagctattttacttcaatggcaggttctatcattaatagactgcgtattggatatttatggcgactaatgttcaatagagagcctaaactttgctacacgcaaggagacgactcattgattggtgatgacgaactaatccaaccagaaagactggctgaattagctgcaccattaaattggaacattaacccgaataagacagagtattctcgtactccagagtttatttcttttctaggacgaacatctagaggaggatttaacacaagagagcttgagaagtgcttacggttattaatctttcctgagcaccctgttccatcgggacggatttccgcatacagagcaaggtcgatcaatgaagacagtggctttaccagcgactatattcagaccgtcgctcgacgattaagaagacgctacggaatagctgaagaatcagaggttccgaagatgtttaaacgttacgttgttaggtag

SEQ ID NO: 309 - DNA, 1437 bp, Beet cryptic virus 3
  source 1..1437: mol_type = other DNA
atggcgtacagaaacattcgtgagtatgagttcactaacttcaatgaagaattgtaccagattgaaggaacccacactaatacgatcggacgagagtccgaggtaattctgaatgacgagttcgcgaaagctatcctcattgacgaatttccagtgctatacgagcaagtctgtcaaggatgggctagaagcttctacaccctagaggggcacatgcaagctattctcgcatacgcacaacccgacactccaagggaaacctttgaccaaaccatttgggatcaggcatacactgccgtccagaatgagttacgtagccttccaaaagcgagggcatttgatgttaatactgaacttgacaaagttccatatgagcagtcctcctcagcaggatatggctatcgtagtcacaagggaccacccggaggcgagacccatatgagggctatcagtagagttaaacctactctgatgacggcgataaggccagatgaagaaggaccggaatacactatactcgaatcagtacctgacattggatacactcgcactcaacttgcagatcttcgtgagaagactaaagttagaggcgtatggggcagggcattccattatattctaatagaaggcacagccgcaagacctttacttgagaactttatgcttggaactactttcatgcacataggttcagatccccagttgagcgtaccacgtatactacaccagatgaaacgcgaaggttctaaatggttatatgcactagattggtctagttttgactccagcgtgacgagatttgagattaattgtgcctttaacttattaaaggaacgtatcgagtttcctaacgaagagacggaattagcttttgagcttagcagaatcttatttaagcataagaagttagccgcccctgacggtaacatatacatgatacacaaaggaataccatccggtagttactacacttccatcgtaggttcagtagtcaatagacttcgaatcgaatatatatggagagtactattttcgagatcaccccatcggtgttacacccaaggtgacgattcattaatcggcgagacttttctcgttgaaccagaaacggtcgcccgcgaagcggcgaagtatggatggataatgaaccctgataagacagaatactctacagatccaggatatgtcacattcttaggaagaacggcacatggatttatgaacgctagatcactagacaaatgtctgagacttctaatgtttcctgagtaccctgtaacttcagggcggatttccgcctatagagcagagtcaattgcaagagattgcggaggacttagcgaggtaataaatctcgtagctcgtagattacgtagacaatacggagtagctagtgaggacgaagtcccacattactttaaacgttatgtagcttaa

SEQ ID NO: 310 - DNA, 1464 bp, White clover cryptic virus 1
  source 1..1464: mol_type = other DNA
atgaatcaagacactcctctcgccaatctcaatggccccgaagttccatccggcaacgtccctcccgccaaccctcctggtcgaacaaacgtcgcccctcccgcccaaggtgctgttcaacagcctcctgctccggctgctcgtcgtgcccgcaacccacacggccctgtccctcctgctggaccatcccgttctgctggtgcccctgctatgcttgaactttccgctgcctaccctatgtacaccgaacaacgtcgtgcacccaactactatgtgcctgacgcacaacttctcttccacaccctcggtgtctgcgaccaactgatgaccacaactgaccgcttcctccgctccatgccttcatggttgccaatcgtctctcagctttacgtctctgtcctttggaatgttatgatactcaaggtctatgtcaacaccggttacggtgctgcctacgcacatgacttggatgtcctcctcaaccaccttcaaattaacgaatgtatgatccctggccccctcgtcccctttttccaatccctcgctgccgtcaatggcccttttgactggataggtgatattattcctgccatgccatcattcactgaactctggactgatgaatttgctccccatgctgcttacgctaggcaaatacccatacctgccatcctccttgatcagctttaccgttttgccactcttgcctttgatgctcaactacaaacaaactatgccacttttgaatggtatagcaacattttcaaccaagatgtcaacactcacaatgctcgtctccgcctcggccctcaactctgtggctctctgttcacaacacaagcccagtgtgactccgcccgcgccttctggaacccagcttttgctaatgggtttactagaatcgatgctgctaatggccccctaatggcttttccccaactacttggatttatttctcaagatggtgccctgcaatctaactggttcatgcacatctccttaatcatgcacaagtacgctcagtacttcaatggttctgtaccccttaagtccatctcccctgttggaattggtgcttccgtgatctacggaactccgcttgaagataccaacgtccgtgactggctctaccccgctgctgctgctattgctcctttccgttctacccgctttttaccgcgtcgtgaactacctgcgaccctagctgtccgtttcgcccatgccgatcatgagatcgaagaacaagctgaacagtattccatactgtgtcacaccaatatgaagtggtatgtcaacaacgccactcagaacaaccacacagctattgaaggaaactacattcaccaaggtgagtactggaacttcacccctttccgctactctcctcctgtctctctgaagacccagtttgctcaagtgatcgcttcccgctaccaccaacaagctgccaaccgtgctgaatga

SEQ ID NO: 311 - DNA, 1464 bp, Vicia cryptic virus
  source 1..1464: mol_type = other DNA
atggaagctcatactcccgctgctgatgtcaatggacccaatatcccctccggtgctgtctctcagccagaaatcgctccacacaatcaagctgcccccaacgtctctggtgctgctcaggctataatagcccccacccctgctcgtaagcgcacgcctcacggacccattcctgctgccacctcatctggcaacacctctgctcctgctctcctggaattagctgctgcttatcccatgtacaccgagcaacgccgttcattcaactttttcatcccagactcccaaatgatgtttcactctttaggtatctgcgactcaatgatgaactccaccgatcgttttcttcgatcctcacccgcttggctccccatcgtttcacaactttacatctctgtactgtggaatgttatgattatccgcatcctctctcacactggatatgccccttccttcaccgatctactcaatactctcactactgaccttcaaattgaagagtgtatggtccccggaccactagtccccttcttccaatccttggcctctaccaatggcccttttgactggataggcgatatcactcctgccctccctggatttgattctctctgggatgccacgaatttcactccgcacgcaagctacacccgcttcgtccccattcctgccatcttgttagaccagctttactactgcgcgacttacgtcggcgttaatcaaggcgacctctaccctaccttcacttggtatagaaacattttcacccgcactggtgcaaacactcctgctctcctccgcatgggcccaaatctttgtggctcacttttcaccacctccaatcaattcgatgctgcccgtacttattggcgcgcctgctttggcactggttttacccgtgttaatgtcaccgctgctgccttcaccaactacctacagctgttaggtctccgttcccaaactggagaaccgcagactgcctggttccagaatgtgaccatggttatgcagaaatatgcacaacacttcaatggttccgttcccctcaagagtatcgacctcactggcattggcgccgtcgcccttaccggcacaccagttaataatactgctgtccgcgactggctctatcctccaaatgccaatatcgaaccgttcaccactggtcgattcaaccctcgtcgtgaaatcccggcccaactccgcatccgcttttctcactccgatcatgaactcgaactccaagctgaacagtacgctattgctgcgcacaccaacattaggtgggctgctaacatagctacacaacatgaaaggaccgccatcaatccggctcatcttcaccaaggtgattactggaacatgacccctttccgtcacaccggacatctcggcctcaagactcagtatgcccagctcatcgcttctcgctatcaccaacttgccgccaaccgtgtcgactga

SEQ ID NO: 312 - DNA, 1518 bp, unidentified
  misc_feature 1..1518: note = unclassified Partitiviridae
  source 1..1518: mol_type = other DNA
atggctcatcgtactcccactaatgccccggctcttcctcaagttgatgggatcaaccccaaccttccccccaacgacccaggaaccgtcgccgccgcccctaatgctaggaacttgcatctcgaaagagaacaagaagctagacaagaccgcgccgccgccgccttcgtccccaacactcgctttgcggtgagaagaccgacgattcgcccctccccggcccactctgcccctgacctagagactgctctctttgattcagccgtcaaccacccccccaccccggtctcctatccgtctacctcatcctacatccccaacttcacttccgccttctactatctcaacaagatggactccttgatggtccaaactctcaactggacaaacaactgctctggatgggttcccccatactcccagatctacatcagcatgcttctctatcttcaagtaatgcgcgccatgaagaaagctggcgtcctcaggcccaactctgaactatcccatctcttcaatgagatgtccacaatctttcctttcgaaagtctgatggttcccggccccctcgtcaacttattcgagaatatcactgcctttcgtcctcttcaaacggactcttttggcaatgtgactcccttccttccagccgaacccggttggtcaaacgcgaccttcttcgctcccaacggctcattagttcgacacctccctcacatacccgctttaatctcccgtctccggagaatttgcgaaaccgcttccgagaatggcttaaatgacatctcgttttccgcccaccaccacggcccggagttcatatcagaactttttggccacatttgtgacaacgatcttcctgaacagctactgctgctcacccctggcctggccacctcctactctggtacactctacctctggcgccaagcccgctctcaactacaaaggtccctcttccctgaggcactcactgtcaacgacgtagtgcccaatacgtggacctccttcctctgcctggacaatgatgattcatggttcagtcccctcgccgctatgatgaacaagtactgccaatattggcatggctcagctcccctctcctccatccccgctgatggatccgctgctggctcaatgatttgcaatgagctcaatggatcctctatttaccggctggctcaatggaccgctcctattatggacccccccgctgacgaggatgccccacctgtcattgccgaacctggctactactaccccaatcccaaccccgtcctcatctttgacgctcgtacctgcattgaagacatctcttctgcacacatgttctccgctatgaccttccatcccaatctcataccatacggagctaacagagctaactttctgaaaggacagttttggaattgccaccccccaagctacagcgctcctgcccaccaggtctatccagctgtatccgcccttatcgctcgtgagtatcacagtgaaactaacctttcatctgacaagtga

SEQ ID NO: 313 - DNA, 1041 bp, unidentified
  misc_feature 1..1041: note = unclassified Partitiviridae
  source 1..1041: mol_type = other DNA
atggctagcccacgtgactctatgatgacgactgctcaagacgctaagaaacgtaaagtttctcatattttgagtccgattcgaatgatcgagcctgctgctcaatccctcgcaatcatcccagttgtccctcaagtccaaatggatgacgctgaagctatggctgaattgctgtatcgtgaacgcgcccgcattgaacttgatattaatcctgcttatatcagtagattggaatcccgtcgagagattggtttctatcctcctgttgcaagtaagaacgttccgttcactactttcaacatcgatgtcgctgaattcagacgctgcataacacatgtacttactgctgcactcaaagtacgttattacgatcgatcaaatcttcgcaacgctgagatcaatactgagataaacacgattgtgcctcgcactgtgtctgcatgcatgtgtgccgtttacgctaaactgcgctcaatacatcgcacccacggtaccctccggaataggtatgtcactactcctacttacaataaggatattgaactgccccttcctttagctatcgctatacaaggtctaggtgccttccgcactgaaagcattctcgcaaaccgagttatcgctccgacctaccctgaggccacccagtatgaaggacgcgctcaagacgctttcaacatgactgagtaccagaactacatcccaactcttaaagatcttggtatcccctgtaaatcagtggaaatccatgacaagaagggaaccgcttggtggagttacaaaatccacaacgaccacggtacatcagatctcgtctgcctgcttcctcctacaaactactctccgcttgctgttgcaacccgaatgctcttcctcgctactcaggacgattctgacgaatgtactgattttatcgaatggccagaagatctcggtgattatggaacacagctgaaagaagccccacccaactccaatgttcgtgcatttcttgccttatgccatggacccctagaagaatggagcaatggacatgcttaa

SEQ ID NO: 314 - protein, 348 aa, unidentified
  REGION 1..348: note = unclassified Partitiviridae
MATDNSDAKTEQSKVTESKGSSPTKSLPAHAADEKAAVGATFKLQSRQPTEPPAIPSVFAQSQSLNDSVGNPDFIAQLRDEGMYGLFLPRDRARRTPTPVTLRPLTYFQSIERTISQSMFHLLQTKTEFNNDDAIQRINTIAEQLAIGACLATHMKLRALIHLDFPDRYNIGSLKRPKTITDLPVVAPFAFAIQQLGYVNIANLTEERRYVPVLPETGHTFGIPTGHNWNPNLYAQAVDYARKFGLHFNVVDYTKKQGTAWWLLRQHFEDGIFELQLPLPEVNFTSSMALTLSLFLNAEEVNATSEIFDLTPVGADIYGYIMREPHLGINVSTFEVIDESAKEIVSNV

SEQ ID NO: 315 - protein, 346 aa, unidentified
  REGION 1..346: note = unclassified Partitiviridae
MAFESKTSASESTVNKDAAPAPSSASVKSPVKPSSSGLGRAKTETPHLPASNYEIPRYTRDTQPVVGNKNYEFSLRMDGRLGHSMDRSKTARPEVNCSLNRANFHEGVLKVLRQILSEMARLKQDLSDAEIQERVESVSLTLAYGVATMAYLKLRAINLFRPNEASKFLTKPKVPDHFEIPTPFAFAISQLGVVEVSSLSRRMICYPTADLADASNHLVGNKRNWSQTAYAEAVRYAKYLGMSFSTVDLDIKVGSSWWLFKPDVTDGLLSIRCPLPEDNYTLAGATVFMLFYHDVGSDPAIDLFNIDSLGNDDYGSFIRNPRDGFNASSYYAISSEGTDEMWKSSA

SEQ ID NO: 316 - protein, 347 aa, unidentified
  REGION 1..347: note = unclassified Partitiviridae
MDTAQTDADKAAGKRGATVPPEGEPAAKTMRFSDAIVPAGPSKDASTVATAALPKDLIQWPIERLATQVNQVFLDTTIQNGEGGYEVPDDQIVPTLRVYEINVTNLRNAFRSVLYTILRAKFIERGDRTVTAATTLATNVSNLTADACMAALYAKLRSLHRQIYTHKGRFTDQPTYTKDVELPLPFAVAIDGIGMFRTSAMSTRFNVVPVYPENTKNEGRSHDSINFLEYKSYLAYFAEIGIPTRTIDTRVTPGNAWWTYRTSYDGDVYDLKINFPPLHFNDHLANLAIMFLGNSGADRNNASIITTKTDDQDYGWYVKDIREGEQARAFAALCQWDPRYWNEKNNV

SEQ ID NO: 317 - protein, 348 aa, unidentified
  REGION 1..348: note = unclassified Partitiviridae
MDKDAAAAQTEQSKITESHGSSPEKNAAAHTADQKAAVGSTFQLQSRKPTEPPTVPSAFAQSTALNVSVGNADYIAEQRDEGKYGLFLPRDRAHREPEPVTMIVGNFFDGIVRTTERAMVYLLQKKSEFNDIDVLQRVHAVAEQLAIGAMLATYFKLRALIYLDLPDRYTLGSLKRPKAIADLPVIAPFAFAIQQLGYVNVANLTRERRFVPVFPEDDHTFGVPLEHHWNPNLYSQAVDYARTLGLHFNVVDYKKKQGTAWWLLRQHYEDHVFELQVPLPEVNFTSAMAITHTLFLEGEAADPSNAVFDLTPVGADTYGYILREPHFGINVTTYQVIDGSAQEIVSNV

SEQ ID NO: 318 - protein, 346 aa, unidentified
  REGION 1..346: note = unclassified Partitiviridae
MASSKRTPPLVAQQPKKRRFETVFDLGPSLGSTSLAMVHVPQPTPMLTDEAEQMAELLFRERSRIELDINPAYVSELELRREVGFSPMIANKTLAFTTYDLRVVEFRRCITAVLTGALKARYFDRSNLRQAEITAEINTIVPLTVSACMCALYSKLRSIHRTHGRLSARYAAAPHYNKDIELPLPLAVAIQEFGIFKTESMIQNRIMAPTYPEATQYEGRAQDNFNITDYQTYIPTFKDLGIPCKSVDPHIKSGSAWWTYKLNNAHGTTDLVCTIPPTNYSDLGVALRSLFLATAADSDECSEIIAWPEGTADFGTNLRENPPNSNIRAFLALCHGPLEEWSNGHA

SEQ ID NO: 319 - protein, 489 aa, Beet cryptic virus 1
MENNTPLANPSGPNVPSAAAPPTPAPPAIPQATPTPGAVSQPPAPPARRSRTPRGPVPQATPGSTSGAPALLELSAGLPMYTVPRRGVNTFVPDAQMLFHVLGICDQMMLSTDRFTRSSPAWIPIVSQLYISVLWMVAILRVFVASGYGALYSSLINDLIGHLRIDECMIPGPLVPFFQSLGAVCGPYEWIGDIVAAFPDFLTLWDAENFCPTADLARTCPVPAIMLDQLHYFATWTIPAEQILYTNFQWYRNIFSLGLGAGNANNRIGPQLCGSLYSPRAQVDSARAFWNAALSSGITRTNAAEANGAFYTYAQLLGFISQNGTLQLDWFQQVAVVMQKYTQYFNGSTPLKSISTIGIGAVAVIGAPTPDPATRDWFYPAATGIEPFLCSRFAPRREIPNTLGMIFSHADHELEEQAEQYAILTHTNIRWSPSVVAQNAWTAVNDGASRNGDYWIMMNYRFSTRISLKTQFAQVIASRYHQQAANRVD

SEQ ID NO: 320 - protein, 374 aa, unidentified
  REGION 1..374: note = unclassified Partitiviridae
MENGANNPPAAGAIVAAVQPALPPGLDASKLNRATEYLYDADKIRPHRARSPKYRRYMQLKKNEIYVQLIDIYRASFSVTWPDFRHLIHAFPPPANHEPQTYYACCYISGWFRDLYYHIRKALKSLSGLAELEYYRDPIPQMSFEYDNFLALLGAAIRPTHIVGNPEDVMYVPLISEATDVNQVNPFGINNYVHQPELFSAIVATMKDRKKFNMVPLPSNYSGRPSWLFDWHAEDQICAPFQPEGNFNNEDIAMAFVLGTACTSLIGMRDRDDWQFYPNANVPAHFNPHTAQRITPRTMYGSYEVRSYETDHDYYIPSNSAGVTAFLSGPSTSRKRSRTTSSSTPGSLVICKYICLYPYRLHYLPVHINISLFN

SEQ ID NO: 321 - protein, 673 aa, Primula malacoides virus 1
MSDTVLQAKTNFLKNKSPVLSVQESKRPKISRDLMPLFQSNTAARSDSSNQWTIDVFPNMVPITMYIMLTSIAHANDVDHRTTSKVSVATICMYHLSIVYGYFLLNDMITRASASAHARPWIETSWKREFADFLLTLPVPEFLTPFLSQFQHFTTDRTKNVFLTPSAAGFDHNIFFGRVFPLNMFSAIHDCTATLPGNTPRIQVLQDLYSRVLYTITAPGFTCLIPDLIGVTIDQTTPTTANYMNSKLYQVYTAVFNPVLFRDFQHRSSLAALSFQSPVFANNHVNAYDLLFSASAANLRELKVVLQVVSTVLDGKVTTSGTLGDFLTKPSGALAIKHGYSTYALPTWSYNTNATKSAVYSAITTLTLTSERDRAQDICFLQRPAAAFNATTEVTDVTYAATATPNQAVQLPTGHQLTRHFPFSLLQVPNGAEPFPRHDNDDLVQFSDFVHTAPSVLVLDTDGELTISAHLPLLAGKIIESFEIDGSTIEMPNVDKSLGLQNCLFADSAVSYRLVRPGSAFHPQPAGSINPPLNRVRPNPRPRLPASSLLYDRTEIMLPNMNNRIVEARAPDTLPGMTRRAAVNFLRYAQSFFGFHTVDSSSNALSLDTIPGMTNGLLHLWSPYSYTPFENDYFPDPDLAESRHYFLSNLRTIFGTDYNLVQAKHPYEALPVV

SEQ ID NO: 322 - protein, 337 aa, Fig cryptic virus
MAAPNFDILEDLIADGRLQITPYVTSTEPKIFDVDFDALWNRINAIIVPIYTNWDQLRFHYPGTTTTVTNNITLDVGHLVTFALWQLYNRLRSVAECFHHPTRTSRMNTRAPISNRMEFPSFLSSMLESISWLRINDGPVDYLALFTAPTGTSNNYGRSTVQVPDNGCYDRLTGKLRALGVQLTPIDMTPSAGSFWPTTQVITEEELYNIVGTFHSSHYVNEDAIRAIFLAGTAHSQPFPDFGMTVGFVTDRTVMDPLAALAAPTGTPRSEEATSAGRPVHGMYNVNHRGIQPAAGEQNPSGCYIIGRGYERRYTCYLARRIAVFEANRIIRYRFMR

SEQ ID NO: 323 - protein, 415 aa, unidentified
  REGION 1..415: note = unclassified Partitiviridae
MADQGNNETPLPVQATQPAANSDRIPITTLPAPRSQAPNKPEYARIADSLEDEYQIGFQRINPRLHRRYVILRSQELHDRLCNLYAPYFQAGYRVFKHVIEAYPLPAGYASQIAYATACYVSSWCWDLYVSIRESVKKLSQTAFLQQYQTEQFHSQDRYDPFLQHLNTVIRPTHIPGGLEDSLYIPLIANHVQWGNQSPFGINHYAINESLVQGLLDVMDLKNNTWRTVPLSHDTLGRPMWLLDWRQNQAYAWFPQENHFSKEDLIAAHILGIPCSPRLGPRDVDDWQFFPGNVMPQHINIAAYERAIPRRFFGAAEYRTISTRMWELPFLSAVAGQMSGAKRPAPESSATGSGYMESPPAVEGQQTAAIMDPGIMQTRYCQYQIIDWCYHGRVILNLNAQKQNQALRSILFSPN

SEQ ID NO: 324 - protein, 673 aa, White clover cryptic virus 2
MSPDENPTLNKTPKNQTVELVFQDTKVPVPSDNFMESIRSNAAAHTDTANQWIIDVFPNMTPILLYVMHYTHLYAQATEYRIHAKSSVYTLCMYFMTIVYGYFLLNDIYTRPARSVHARIWIESSWRSEFVKFLTTLPVPDILKPILSQFHPFTTARTKNVFFCPSAAGFDHDQFFGRTYPMNMFAAIHDCTATLPGNSTRTDVFRDLYSRILYSITTPAYTCVIPDLIGATPDQTTRTTINFMNSRLYQTFNSLFNPVLFRDSQRRAALAALSFTAPTYPTNHINAYDYLFSATAPNLRELKIVIQSVIACSKGVFDVSGTLGSYVSDYSSSSIVHHGYSTYPLPVWSHTDDATKPQTFTAATIFQLVTEDARAQDICFLQRPAAEIAHTRRITDLTYASTATPTVPATMPANHGIFRIWPYCLRCDNDAENGFPRHDNEDLVAYSDEIHTAPSVLVLDTPGDTVISAHMPTVVGKVIESFELDGSTIAMPDARKSLAMQNCMFADSAIAYKYVRPGSYWRPRAAGSILPPLNRALPTARPRLPASSLLHDRTKVFLPQLNAHVNAIPENNLLPGFTQLTPVNVIRYVQPFFGFHTVDRTANDAALDAVPGMAEGHLMIWSPYTYNPYESDDYPPVALDASRHYYLTNLRTIFGTDYNLVSVKHIYESYPAV

SEQ ID NO: 325 - protein, 673 aa, Red clover cryptic virus 2
MSTEETLPPTKTIKNQNVNLIFQDTKVPIPSDNFMELLRSNAAAHTDTSNLWVIDVLPNFTPILMFIMHYAHIHAQAADYRVHAKSSAYTLCMYYMTVVYGYFLLNDLHTRPTRSVHARVWVESSWRSDFVKFLTTLPVPEFLKPILSQFHPFNTDRTKNVFFSPSAAGFDHDQFFGRIFPMNMFAAFHDCTATLPGNSSKVDVLRNLYTRNLYTITAPGYTCMIPDLIGITPDQAAATTVNYMNSKFYQTFTSIFNPVLFRDSQRRASLAALSFTAPTYPTNHINAYDYMFSATAPNLRELRIVLQSIIAITKDVFKSDQTLGNYVADYSSGSIIHHGYSTYALPTWSHTPTTGKFDRFNTANTFNLVTEDARAEDICFLQRPAAEIPHTRLITDIVYAATTAPATSLTVPANHALYRRWPYCLRLDEDAANGFPRHDNEDLVAFSAESHAAPRVLVLDTPGDTVTSAHLPTLTGKVIESFELDGSTIEMPDARKSLGMQNCMFADSAIAYKYVRPGSYWRPRAAGGTLPPLNRAPPNSRPRLPASSLLHDRTKVFLPQLNRHVNAVPDNDALPGFTQITPVNVIRYIQSFLGFRTVDSSANEAALDAVPGMNESLLMIWSPYTYNPYESDDYPALAYDASRHYYLTNLRTIFGTDYNLVATKHIYESFPAV

SEQ ID NO: 326 - protein, 673 aa, Hop trefoil cryptic virus 2
MSTEETAPQTKAIKNQNVVLSFEDGKPPGPVSDFMSLFRSNSCAHTDTANQWIIDVFPNMTPILMYVMTKAQHHSEATDYRLHSKSSVFTICMYYMSIIYGYFLLNDLHSRPTQSAHARNWVENSWKSSFAKFLMTLPVPEFLLPILAQFHPFNTDRTKNVFFSPSAAGFDHDQFFGRVYPLNMFAVIHDCTATLPGNSTRTQVLQDLFSKVLYTVNAPGFTCLIPDLFGLTLDQTVNTTVNYMNSRLYQVFSSLFNPVLFRDSQRRSSLAALSFTPPTYTTNHINAYDYMFAAYPANLRELQVVLQSVSAIIKDVAECKIPLGTYVADYSSPSIIHHGYSSYALPTWSHSETEQKVDRFENATTFNLVTEDARAQDICFLQRPTQAIPHEHNVTDLIFTAAGAPDTDIPLPANHAIVRRFPFCLRLDAPAENGFPRHDNEDLVKFSDDTHSAPGVLVLDTIGDGVLSAHLPTLTGKIIESFELDGSTIEMPDVRKSLGMQNCMFADSAVAYKYVRPGSYWHPRAAGSTLPPLNRAPPNSRPRLPASSLLHDRTKVMLPQLNVRINAALNDNALPGLTRVTPVNVVRYTQSFLGFRTVDSSNNAANLDAVPGMTEGLLMLWSPYTYTPYESGDYPFADLSASRHYYLTNLRTIFGTDYNLVSVKHPYEAFPVV

SEQ ID NO: 327 - protein, 673 aa, Dill cryptic virus 2
  SITE 373: note = misc_feature - Xaa can be any naturally occurring amino acid
MSSTSLTSRLNALKTKTMNLTFTDVKAPVPSADILELFRANTGARADSCNDWTIDVFPNMTPIMMYTMLMSIRHANATQQREHSKASVATICMHHMAVIYGFFLVNDLHVRPAPSAHARSWAETSWKDDFVNFLMNVPVPEFLTTILSQFHAFETDRTKNVFFVPSAAGYDHDQFFGRVYPLNMFATIHDCTANLPGNSSKIDVLKDLYSKVLYSIQVPNFTCCIPDLIGVTISQVTNTTANHLNSKLFQVFNSLINPVLFRDFQRRSTLAALSFKSPTYQTDDINAYDLLFSATPANLRELKVVMQAVSAVLADSVPCKSTLGRFIAENSSSAIIKHGYSTYALPTWSHNENSNKSSLFAAVTTHNLVSEEXRAEDFCFLQRPSQEIPHTHELNDLRYVADTDKSTAVALPANHSIIRTFPLCLRYNSSQKDGFPRHNNNDLIKFQDEIHTYPSVLVLDTDGDLTVTAHLPLLAGKIIESFEIDGTTIEMPNTFKSLGMQNCMFADSAIPYKYVLPGSYYHPRTTGTVLPPLNRAPPNSKPRLPASTLLHDRLKIMLPRFNVAIAEQNIGNTLPGMTPIAPVNVLRYIQSFLGFRTVDASSNAATLDAVPSMPTDRLMVWSPYTYTPYEPDDYPIPDLGASRHYYLTNLRTIFGTDYNLVQTKHPYEALPVV

SEQ ID NO: 328 - protein, 490 aa, unidentified
  REGION 1..490: note = unclassified Partitiviridae
MDPNVPIAQPNGPAIPNVAAAAANPPPPIQPAPPAPGAVQQAPAPAPRRNRLPHGPVPTSSRTAVTSAPALLELAAATPMYTEQRRATNFFVPDSQMLFHVLSICDQMMNSTERFLRSSPAWMPIVSQLYISVLWNVMILNVFVNSGYGSAFSSFLSELYGVLRIDECMIPGPLVPFFQALAAVNGPFDWIGDIVPGLPNFSALWNEAGFYANSNYARQVPIPALILDQLHYFSQYAIPANQQSTYATFEWYRNVFQQGLGAMNRLNRIGPQLCGSLFTTQAEYDAARNFWNASLNTGITRANAAEGQPAFNNYRQLFGFESQLGALQLNWFTSVSIAMNKYTQFFNGSVPLKSILPTGIGAVVIAGSPVNNPSTRTWIYPPDTSIEPFTTTRYPPRREIPDAMAIQFAHSDHELEEQAEQYAMLTHTNIKWHANLTAQNNWTAITADGLYLGDYWNMMPYRFFAPLHYKAQFAQIIASRYHQQAANRAE

SEQ ID NO: 329 - protein, 487 aa, unidentified
  REGION 1..487: note = unclassified Partitiviridae
MNHNTPPADANGPALPEGSVPPPNPPTHIPVAPAAPGAVQTPPAPPARRNRQPHGPVPAASNHHAPGAPALLELSAAYPMYTEQRRGVNFFVPDSQMLFHVLGMCDQMMNSTDRFLRSSPAWLPIVSQLYIAVLWNVMICRVYINTGYASHLAPIIDVLISHLQIHECMIPGPLVPFFQSLAAVNGPFDWIGDITPAMPAFASLWNAEQFTARDSYARQIPIPAIILDQLHHFATRAIVAGVANYGTFEWYRSIFNRGVGATPANSRLGPQLCGSLFTTNAQFDAARAFWNSALASGITRTDAARPPLVNYAQLLGFQAQDGSDQLNWFQFTSSVMQTYCQYFNGSVPLKSISLTGIGAVVTYGIPVNNVATRNWFYPTAAMIEPFATSRYAPRREIPNQLAVKFQHADHEVEEQAEQYAILTHTNIKWYESLITQNALTAVTGNLVHQGDYWRMMPFRYSSPISLKVQYAQIIASRYHQLAANRAE

SEQ ID NO: 330 - protein, 394 aa, unidentified
  REGION 1..394: note = unclassified Partitiviridae
MTTGDRVTPTDGITTVDAPTATLEAKLDSSSTAYLKSATDFLTQSSRIGTLRTSYKDRRRYVYLKSQAMFDSLVELYSLLFTTQWQVFRRFTDIFVPPANVTATRYLAMNYISMWIQDTYAMNRAALMKLSVVALNDRFPVEIPIVGYQYDAYLAHLSAALRPTLTKHMMEDTLWIPCFTRSPNFQVESPFGITGYVINDVIFTGAMNIMKERKLWRIIDLPTNVLGRPSWLFDWHDDDRVCAWFPMEGNFTMDDVTMAYILGVACTPKLAPCDVDDWQYFPNDVVPANLDPYAFLRLTNRRFHGGYEIRTLDAANMPGQTGTTTDSRKRPRPGESTSAPALPQTSEPQTQMQVDTPPAPKPRFRLVDWTYHYLVVLNKEDHTRFAALKSICWN

SEQ ID NO: 331 - protein, 487 aa, unidentified
  REGION 1..487: note = unclassified Partitiviridae
MSMKRKSRPSSSTKDLEVEDTYLKQSGLDSMNKLEPVEQSKDEETTKVSMLPTASTAIIAPRKLTAEDFSSKRKPDQTSAVSPFFGFLRTHILHPTQGRLSHYYPSCHMMDYILHSINSTLCDNYYFKRETPNYHPYILRLYFGVLFWVQCLRAGNDVQVINDLHYDFLQRFLDCNPLESLAIPGPLLGLFKTLCSSQPEFPHYGKVYPRIPASPGPRRRDMFSKNVPSAQFLPNVPGIFALIHHLHGLSEGEHPIYPKRKRHIPVTEEASNFGFKAFAAFPNRIQRDRWMVSSPGLQYPCEADMKMNEAFAERFYDFDFPAFNADDNLSTITNFLHMRKSMAWFIRAKEVACSAARFFSDSGTLADCSPHGLVSNQIIVAITPPPEETFADPRFSADPRALYPFSFKLKSTAHNLPPLAEAAAAFSQTHIRIFPEYPLAGNFGQKTDESGPFWDIRPIGSSPTDDTSYLTIPPMVKAALIEKGSSR

SEQ ID NO: 332 - protein, 672 aa, Cannabis cryptic virus
MSSSTITSRLDALKSKNVIIEFKEAKTPVPSADILQLFADNTAARSDSSNEWIIDVFPNFTPILMYIMLNAIRHANATAQREHSKSSVATICMYHLSIVYGFFLINDLNVRPAPSAHARIWAESNWRSEFVKFLSTLPVPEFLIPILSQFHAFETARTKNVFFIPSAAGYDHDQFFGRVYPLNMFAAIHDCTATLPGNSSKIDVLRDLYSRVLYSITDPAYTCVIPDLIGITLSAADITQAQYMNSKLYQVFSSVINPVLFRDFQRRSSLAALSFKAPTFASANVNAYDLMFSATSANLRELKVVLQSVHAVISDDVSCKTSLGQFIADATTSTIIKHGYSTYALPTWSHSEVTNKAQTFRAITAQQLISEDARAQDFCFLQRPAAEIPHTHNLEDVRYVRTDAPTEPVALPQNHSIIRNFPLCLRNNANAAVFPRVDNNTLINFSEEIHTVPSVLVLDTDGDQTITAHLPLLAGKIIESFELDGTTIEMPNAMKSLGMQNCMFADSAIPYKYVRPGSAYHPVNAGTILPPRSRATPNSTPRLPASSFLYNRLRVNLPRFNLAINEERIGNILPGMTLLDGVTVLRYVQSFLGFNTVSRADNADNGDNVPAMPRNLLLVWSPYTYTPYESDDLPEPALGESRHYYLTNLRTIFGTDYNLVQAKHPYESLPVV

SEQ ID NO: 333 - protein, 488 aa, unidentified
  REGION 1..488: note = unclassified Partitiviridae
MNPETPLADPNGPTPPHGAAAQPTPGQHIPAGTPSTGAVHLTPAPQPKRNRAPRGPFDNASSTSAPGAPALLELAAAYPMSTEQRRSPNTFVPDAQMLFHVLSLCDNMMLSTDRFTRSAPAWLPIVSQLYISILWFTMILKVYVNSSYGAMHAHLLNILVAHLRIDECLIPGPLVPFFQSLGAINGPFEWIGDIVPIIPEFSQLWNTTSFYPTDSFARIFPIPAILLDQLHYFASITVTEQETIFGHFEWYRNIFSLGQGTFNPRLRLGPQLCGSLYATESQVTNARTFWSAALRTGITRTNAAANQPLFSSYPQLLGFVDQNDAMQLDWFQHTAIVMQKYCQYFNGSVPLKSISPTGIGAVAVHGYPNYSGAARTWMYPATNTLSSFKSSRFSALREIPALLTVRFEHADHELEEQAEQYAIATHTNMCWSKNLTTQNNWTQVAEANVHSGTYWSMTPHRAHASVRLKTQYAQLVASRYHQQAANRV

SEQ ID NO: 334 - protein, 430 aa, Pepper cryptic virus 2
MATPVSDTPTTKSTQDKTDQLPAATPPITQKPPSYKSEISRIADYLADDYEIGYVRDRPPKHRRYAVLRTQALFNKLIEIYGKLFKQNWEFLRRSIDGFTTAVGIRERQWENAAHAYISCWFFDLYVSVRNAVEKVSGTAFTQYYHAHAAHILHEYDALLVHLNAIIRPTHIKLALEDCIYIPRIATNMNITNPNPFGLTEATWDRDADFAYSIMDRMKAPGSRWRVEPLVTDTLGRPGWLLDWHQVGAQQQAYAWFPSEGNYSMEDLIAPHIIGEALTPLMGPCDIDDWQYFPNFIRPANVPWQNMERVSARRFRGSAEVRTYAEEKVNLTRFTSTGHETSKKRSKPSSTTTTSSQGGSRDMETEDEPREVVTDITLSQVTSTQQLQTLMTTGEADRFRIIDYTYYCCVILRNTAQKRDGAIRSFCFKQ

SEQ ID NO: 335 - protein, 411 aa, unidentified
  REGION 1..411: note = unclassified Partitiviridae
MAENDIPAEGIPARPPPAANPFTGLNRDELDRIVEQPEEDNQIGLFRDKARSYRRYVDLRTSPLSTRLINLYSNLFTTHWNVFKHTMSGEAPAGFASKAIYLATIYVSAWIWDLHVSIRESVRRISGTAFNQLFQQDAYHQADRYDPFLQHLNTIIRPTPIHQALEDTLYIPVVSRNIDWANANNNVFGLPSHAYDDNILNAILDVMDDRRNQWSTIPISTNVLGRPGWLLDYRLGQAYAWFPMEANYSQVDLIAPHILGTPCTPRLGPRDKDDWQVFRYNAAPPPGVRLNPHDFRRATERSFYGSAEYRTIEHAVHVFDWQATIPPPDAATSAASQPGKRKQGDSSTSAMALMETDKDPPQPFVPPPPVDLNRYRLIDWCYHARVIDGEDQQTRNRALRNFIFQAGHQPT

SEQ ID NO: 336 - protein, 412 aa, Pepper cryptic virus 1
MGDRVNAQDDDTVVPHQAPLQPAALQQDLTRSADYLLDNVRIGNHRQRYDKYRRYVLLRSSEIFTSLVAIYAHIFSSYWQHFRRFTDQFQAPTGVQLPTFVARVYISTWLHDLYCSIREATRSISPLAFNERYSYELLPYSTEYDPFLAFLSMSIKPTHIQHTPENTLWIPILCENYDWDRNEANHNPFGITNFTLNSNLFYGLLAILKERKEFKLSTLTTNTIGRPCWLFDWHDNVQVCAWFPREANFNSQDVTAAYIIGVACTPKLGPSDDDAWKYYASLNSVPTFTPTEPRLTNRRSYGAYEVRTRETENNYFLPDSLLNIIEDFTATGTTQRRKIRRPSATSASTGAAIIIRDTPGTASTATTSTTETEVTFPPVIRTKIRDWYYHSRVILELEDNSRTAALRMFIIA

SEQ ID NO: 337 - protein, 490 aa, Carrot cryptic virus
MDDNTPAAQPNGPHAPAAAAPHAVPPPAIPPATPAPGAVQQPPAPAPRRSRNPHGPTPISSRVSAPSAPALLELPAATPMYTEQRRSANFFVPDAQMLFHVLSICDQMMNSTERFLRSSPAWMPIVSQLYISVLWNVMTIHVFVNSGYGSAFSSFLTELYSVLRIDECMIPGPLVPFFQALGAVNGPFDWIGDIVPGLPNFSALWNEAGFHANNNYVRQVPIPAVILDQLHYFSQYAVPANQQSTYTTFQWYRNVFQQGLGAMNRLNRIGPQLCGSLFTTQAQYDAARNFWNASLNAGITRANANEGQPAFSNYRQLFGFESQTGVLQVNWFTSVSIAMNKYTQFFNGSVPLKSILPTGIGAVVIYGTPMNNPSTRTWVYPPDTGIEPFTTTRYPPRREIPDALSIQFSHADHELEEQAEQYAMLTHTNIKWYQNLETQNNWTAIAAGGLYLGDYWNMMPYRFSAPLHYKSQYAQIIASRYHQQAANKAE

SEQ ID NO: 338 - protein, 674 aa, Crimson clover cryptic virus 2
MTTNSSNARLNKLKNNTVPLPFNATEVPEPATDLLTSMKENTSAHSATANQWTIDLFPNMIPVLIHVMSAASHHANAINFREHSKTSAATVAMYHMAVVYGYFLLNDLNIRLNPSAHARSWAEASWKSEFVEFLYGLPVPEFLAPILSEYQHFVTDKTPNVHFTPSAAGFDHDQFFGRVFPLNMFAALHDSTATLPSSSTYAQVLQDLFSRPLYTITDPAFTCYLPDLLGITLTAGENVTTLNYINSKLYQVFTTIFNPVLFRDHQRRSSLAALSFKSPTYPNPNINAYDLLFSASVANLREIKIVLQSIYKLFDGRVACKHTLSQFICESTSPSITKHGYSTFPLPTWSHTESDAKAIRFSGVTALVHVSEEDRAQDFCFLQRPAEAIPHVNEVPDIRYATTEDPETAVPLPANHILVRRFPFAIRLRQEAANGFPRHDNDDLTKFSDHVHTAPRVLILDTTGDGIISAHTTTASGKIIESFELDGSTIEMPNADKSLGMQNCLFADSAIAYKYVRPGSFYRPRTQGSVLPPLNRAPPNSRPRLPASSMLHDRTKVFLPQLNTRINEAVDIRTLPGLTMLNNVNCINYAQSFIGFKTVDATRNDDVLDAVPGMPLNNLILWSPYTYTAYEDETYPDPVFSNSRHYYLTNLRTIFGTDTNLIKAVHPYEAYPVS

SEQ ID NO: 339 - protein, 426 aa, Beet cryptic virus 2
MTGPTQAHLIEATSFTGLNRDELDSIVDALQEENQIGTFRDRPKAYRRYVHVRSQELFDRLSELYADLFTSNWSAFKHAYITEPTGREDANSISTKASFMAQCYLTAWFWDTQYCIRESVRKLSGTAYNQHFTEDLNLISPKYDPFLQHLNMVIRPTLIHQSTEDTLYIPLLGQTFNYNEDAYNFLNLQGCGTEIRQVYAITDVMDSRRTEWSTIPLATNVLGRASWLLDFKQGNAYAWFPFESNFTELDLVAPYILGIPCTPRLGPRDEDHYQHWTHNDPPLSTDGNIILNPLAYVRSSERKFFGNAEYRTMEYNSYTQNFEAYMPRVTTSTQAKNKRKKDAAPEQLSTGQGDGSSSSKDAEMSTEESEAPPPPKIPRPRQIHQFRLIDWVYHSRVILGADKAMQRRALWDFIHAGATPSHQPRP

SEQ ID NO: 340 - protein, 393 aa, Beet cryptic virus 2
MAPNDELAANLQGLKRPALDEIVHYLEDRNQIGISHDKNLSYNRTCVLLTNELHARLVTLYTNLFTTSWRHFKSAVRKEMPRTEANPQRWLANCYITAWFWDLQASIQEATKTLSGKIYQDYFRDNIHPILDRYDPFLQHLNTIIKPTHIVNATEDVLYFPIISADYRRADADMNIHRITGAFTRPNVVMDLVSLMDDPNSGWSTVPLNTNVFGRPGWLLDYDGTDAYAWFPMENNYNMCDLIAPHILATPCTAKLGIYDADIWQNCPGNIPITATTARTARRESERRFYGSAETRTIEQRNYTVNFPELLKPRDTGARALVTTTQRASTSGSVEPTTGAQPEPETPTFQITFRQFRILDYCYLAKVIHKSNSQMINKALRNFIQKGASKKTE

SEQ ID NO: 341 - protein, 491 aa, unidentified
  REGION 1..491: note = unclassified Partitiviridae
MDKSKKYLSDAKEIIKANGLEEKFLAAAGLSSMDELDPKNFAEFEAPAPPQVMKPSIAAAPIIQKPLSKQVADASTGFNPESASDMLRPFIGLKINYKARKTVSTYAPSSLMMDYILHQVNNLLVDNFYFRRACPDYHPYILRLYYGVLFWIQCMRAMNHARLLHAEASQCLSTFLDAFPADSLPISSPLLDLFKTLCCSQPEIPTFGVVSPTLPFHPGPEARSAFMLADATSHVLPNIPGIFALIENLRTCLNPGDGQQPNLPAKGKHIPVTSTAAAATVFGHHQFPILANRSNAEKWSLCSSGLQYPCEADRRLHENFSERLDNFEFPVTAEDDDLRFYSDFLHITNSLAWFAQVRDVAANEAAFFNGSGTLADCPPFGIAANQITAGYLAPIIQVAAPTRSADPASLFPFAIKLETASRALPELAEAMAALAQTNVNIYPTHPYYNHIGAESRRGPFWNLAPVESSITDHSSYLSLREVVRGLMKPKG

SEQ ID NO: 342 - protein, 483 aa, unidentified
  REGION 1..483: note = unclassified Partitiviridae
MAALGDGVPPIADNNNPQGRDANPIRGGTDPLRRATDWLEDNSKITMTFHRHANYRRYATLRLQEMFNALVQLYQNIFMSHWHHLRRTVENYQVPAPLAQTPWLYVARLYISVWIVDLYASIRRACLRVCPEAYNEYYTQDYQQVVFEYDAFLSQLLSMIKPTVVNLVHETTLFVPRLAEDQPFPADDMNIFGLDRFDYNENVVHGVISILRDRKLMKFEKPSETGFGRPFWLFDWHRTGHAYAWFPAENNYDMRDVTVAYIVGVACTPKLGPRDIDDWQHFPGNIIPEHPRAEDHVRVRPVAYHGAVEVRTIDVQQWTAPPEVMSLIQVTLPPAAAQQQQQQQPAAQAQPAPARQQPPAQQQQQEQGLIRYNLRQGFKRRRTDPSTSNDNDMPVEDIEEPPQPEQGEDPIHPEEEAAPPVEPQPDPAPAPAPAPQAPAEPIITMNRVRRFRIITYCYYRQVTDNEDLHSRLAALRQVDSLIA

SEQ ID NO: 343 - DNA, 1047 bp, unidentified
  misc_feature 1..1047: note = unclassified Partitiviridae
  source 1..1047: mol_type = other DNA
atggcgactgacaatagcgacgctaagactgagcaatcaaaagtcacggagagcaaaggttcttcaccaacgaagagcttacccgctcatgctgctgacgagaaagccgctgttggtgctaccttcaagctgcaatccagacaacccactgagccacccgctataccaagcgtattcgctcaatcacaatcacttaatgattctgtgggaaacccagactttatagcgcaactgcgagatgaaggcatgtacggcctgtttctgccaagagatcgagcccgtcgcacgcctacaccagtaactctgcggccgttgacctactttcaaagtattgaaagaaccatcagccagtcaatgtttcacctgctgcaaacaaagacagagttcaacaacgatgacgctatccaacgcatcaatacaattgctgaacaattagcaatcggtgcctgcttagccacacatatgaagcttcgagctctcatacatcttgattttccagatcgctacaatataggaagtctcaaaaggcccaagaccatcacggatttacccgtggttgccccatttgcctttgcaatacaacaacttggttatgtcaacatcgccaatctcactgaagaaaggaggtatgtccctgttttacccgagaccggacacacttttggaattccgactggacacaattggaaccccaacctgtatgcccaagctgtagactatgcaagaaaatttggtctccactttaatgtggtagactacaccaagaagcaaggcactgcctggtggctgttacgccagcactttgaagacggaatcttcgagctgcaactcccacttccagaagtgaactttacttcttcgatggctttgaccctatctctgtttctaaatgcagaagaagtcaatgctactagcgagatattcgacctcacccctgttggagctgatatatacggctacatcatgcgcgaaccacacctcggtatcaatgtttcaacatttgaagttattgatgaaagcgccaaggagatcgtatctaatgtttaa

SEQ ID NO: 344 - DNA, 1041 bp, unidentified
  misc_feature 1..1041: note = unclassified Partitiviridae
  source 1..1041: mol_type = other DNA
atggcttttgaatccaaaacatccgcttccgaaagcactgtgaacaaagatgcagctcccgctccgtcctccgcaagtgtcaaatctccagtcaagccctccagctctggactcggccgcgccaaaacagaaacgccccacctcccggcatctaactatgagatcccgcgttacacacgcgatactcagccggttgttggaaacaagaattacgaattctctctccgcatggatggacgccttggccactcgatggaccgctccaaaacggcccgccctgaagtcaactgctctctcaaccgtgccaacttccatgaaggtgttttgaaggtacttcgccaaatcctctcagaaatggctcgcctcaaacaagacctgtctgacgctgaaattcaagaacgcgtcgaatctgtctctctcaccctcgcttatggagttgccactatggcttatcttaagctaagagctatcaacctcttccgtcccaatgaggcttcgaaatttcttaccaagcctaaggtcccggatcatttcgagatcccaactcctttcgctttcgctatctctcagctaggagtcgtagaagttagcagtttatccagaaggatgatctgctacccgactgcagatctcgccgatgcgtcaaaccacctcgttggtaacaaacgcaactggtctcaaactgcctacgccgaagctgttcgctacgcgaagtatctaggcatgtcgttctcgacagtcgacctggatattaaagttggctcctcctggtggctattcaagcccgacgtaactgacggacttctctcaatacgctgcccacttccagaggacaactacactctcgcaggtgccaccgtttttatgctcttctaccatgacgttggatcggaccccgcaatcgatctcttcaacatcgactctcttggcaatgatgactacggctcattcatccgcaacccacgcgatggattcaacgcctcgtcctactacgccatctcttcagagggcaccgatgaaatgtggaaatcgtccgcttag

SEQ ID NO: 345 - DNA, 1044 bp, unidentified
  misc_feature 1..1044: note = unclassified Partitiviridae
  source 1..1044: mol_type = other DNA
atggatacagcacaaacagacgctgacaaagctgctggaaaacgtggagcaactgtgccgcccgaaggtgagcccgctgcaaaaaccatgagattttccgacgctattgtcccagctggtccgagcaaggatgcttctactgttgcaaccgccgccctgcccaaagatcttatacagtggccgattgaacggctagctacacaggtcaaccaagtctttctcgacaccaccatccagaatggagaaggtggatatgaagtccctgacgatcaaatcgtcccaacacttcgtgtttacgagatcaacgtgaccaatctgcggaacgcttttcgctccgtactgtacaccatcttgcgcgccaagttcattgaacgaggtgaccgcactgtaaccgccgccactactttggcgacgaacgtctcaaacttgactgctgatgcctgcatggctgcgctctacgctaagcttcgctctctgcatcgacagatctacactcataaaggaaggttcactgaccaaccgacttacaccaaggacgtcgaactcccgctcccttttgctgttgcaattgacggaatcggtatgtttcgcacctctgcgatgagtacccgttttaacgtggtgcccgtttatccagagaatactaagaatgaaggtcgttctcacgactctatcaacttcttggagtacaagtcgtacttggcttacttcgctgagattggtatccccaccaggactatcgatactcgagtgaccccaggcaatgcttggtggacatacagaacctcctacgacggcgatgtctatgatcttaagatcaactttccgccgcttcacttcaacgaccatctcgcaaatcttgcgatcatgtttctcggtaactccggtgctgacaggaacaacgcttctatcatcacaaccaagacagatgaccaagactacggctggtacgtcaaagacatccgcgaaggcgaacaagctcgcgcttttgctgctctctgccagtgggacccccgctactggaacgagaaaaataatgtctga

SEQ ID NO: 346 - DNA, 1047 bp, unidentified
  misc_feature 1..1047: note = unclassified Partitiviridae
  source 1..1047: mol_type = other DNA
atggataaggatgctgccgccgcccagactgagcaatcaaagattacggagagccacggatcttcacctgagaagaatgccgccgctcacactgctgatcagaaagccgctgttggatcaacctttcagctgcaatcccggaagcccacagagccacccactgtaccaagtgcgttcgctcaatctacggcacttaatgtgtctgtgggtaatgcagactatatagctgaacaaagagatgaaggaaaatatggcttgtttctaccaagagatagagcccaccgtgaacccgaacctgtcaccatgatagtcggcaatttctttgatggaattgtacgaactactgaacgtgctatggtttaccttctacaaaagaagagcgagttcaatgacatcgatgttttacaacgcgtccacgccgttgctgaacaattggccattggtgcaatgcttgccacatactttaagcttcgcgccctcatttacctagatctccctgatcgctacactttaggaagtctcaaaaggcccaaagcaatcgctgacctaccagtgatcgcccctttcgcctttgcaatacaacaacttggttatgtcaacgtcgccaatctcaccagagagaggaggtttgtcccagtctttccagaagacgaccacacctttggagtcccccttgaacatcattggaaccccaacctgtattcccaagctgtagactatgcaagaactcttggtctccactttaatgtggtagattacaagaagaagcaaggaaccgcatggtggttacttcgccagcactacgaggatcacgtcttcgagctgcaagtcccgctacctgaggtaaactttacttctgcgatggctataactcacactttattccttgaaggagaagctgctgacccatcaaacgcggtattcgatctcaccccagtcggagccgacacatacggctacattctgagagagccacacttcggaatcaatgttaccacataccaggtcattgacggaagcgctcaagagatagtttcgaacgtgtaa

SEQ ID NO: 347 - DNA, 1041 bp, unidentified
  misc_feature 1..1041: note = unclassified Partitiviridae
  source 1..1041: mol_type = other DNA
atggctagctccaaacgaactccgccgcttgttgcacaacaaccgaagaaacgccgtttcgaaacggtcttcgatcttggacccagcttaggatctacatccctcgctatggtacatgtgccacaaccgactccaatgctaacggatgaagctgaacaaatggctgaattgctatttcgcgaacgctcccgaattgaattggatattaatcctgcttatgtcagtgaactcgaacttcgtcgtgaagtcggattttctccgatgatcgctaataaaactttagctttcactacttacgacctacgagtcgttgaattcagacgctgcatcaccgccgtccttaccggagccttgaaagcacgctattttgacaggtctaaccttcgtcaagctgaaatcaccgctgagataaacacgattgtgcctctcacggtgtctgcatgcatgtgcgctctttactcaaagctgcgttccatccatcgcacacacggtcgtctatcggccaggtatgccgctgctcctcattacaataaggacatcgaattacctctacctcttgctgttgctatacaggaatttggaattttcaaaactgaaagtatgatccaaaaccgaatcatggcaccaacataccctgaggccactcagtatgaaggacgtgctcaagataacttcaacatcacggactatcagacttacatcccgacctttaaggacctcggaattccttgcaaatccgttgatccccatataaagtctggatccgcttggtggacttacaagctcaacaatgcccatggcactaccgatcttgtctgtactatcccaccaaccaactactccgatcttggtgttgcgctgcgctcactatttctggctactgctgctgactctgacgaatgttcagaaatcatcgcctggcccgagggcactgccgactttggcaccaatctccgcgagaacccacccaactccaacatccgcgcatttcttgccttatgccacggacctctagaagaatggagcaatggacatgcataa

SEQ ID NO: 348 - DNA, 1470 bp, Beet cryptic virus 1
  source 1..1470: mol_type = other DNA
atggaaaacaacactcctctggctaaccctagcggccccaatgtaccctccgctgctgccccccccacccccgcccctccggcgatacctcaggctacccccactcccggtgctgtctctcagccccctgcccctcctgcccgccgaagccgtactccgcgtggccctgttccccaggctactcctggctccacttctggtgcccctgctttgctggaactctcagccggccttcctatgtacactgtgccccgtcgcggtgttaacacctttgtccctgatgctcagatgctattccatgtccttggaatctgtgatcaaatgatgctttcaactgatcgctttacgcgttcatctcctgcttggatccccatcgtctcacagctttacatctctgtactctggatggttgctatcctcagagtctttgttgcctccggttacggagcactctactcttctcttatcaatgacctcatcggtcaccttcgtatcgatgagtgcatgattcccggacctctggtcccgtttttccaatctcttggtgccgtttgcggcccatatgaatggattggcgacatcgtcgctgcattccctgacttcttgactctttgggacgcagaaaacttctgccccactgctgaccttgccaggacctgccccgtccccgccatcatgcttgaccaacttcactattttgctacctggaccattcctgctgaacaaattctctacaccaactttcaatggtatagaaacatcttcagtttaggccttggcgctggcaatgcaaacaacagaattggtcctcaactttgcggctctctttactcacccagagcgcaggttgattccgcccgtgccttttggaacgctgcactctcttctggtatcaccaggactaatgctgccgaagccaatggcgctttctacacttatgcccaactgcttggatttatcagtcagaatggaactctccaactagactggtttcaacaggttgccgttgtcatgcagaagtacacacagtacttcaatggctccacaccccttaaatccatatcaactattggcatcggagccgtcgctgtcatcggcgctccgacccccgaccctgctactagggattggttctaccctgctgccactggtattgaaccgttcctttgcagccgcttcgccccaagaagagaaatcccgaatactctcggtatgatcttctcccatgctgaccatgaactcgaggaacaagcagaacaatatgctatcctcacccataccaacatccgttggtctccttccgtggtcgctcaaaatgcgtggaccgctgtcaatgacggcgcgtctcgcaatggtgactattggatcatgatgaactaccgtttttcaacgcgtatttcactcaagacccagtttgctcaagtgatagcctcacgttatcatcaacaagcagcaaaccgcgttgactag

SEQ ID NO: 349 - DNA, 1125 bp, unidentified
  misc_feature 1..1125: note = unclassified Partitiviridae
  source 1..1125: mol_type = other DNA
atggaaaacggagcaaataatcctccagctgccggcgccattgtcgcagcagtgcaacctgcactccccccgggtctcgacgcttcgaagcttaatcgtgctactgaatatctgtacgatgctgacaagatcagaccccatcgagctagatcaccaaagtaccgtcgctacatgcagctcaagaagaacgaaatctacgttcagctcatcgacatctatcgtgcatctttctctgtcacttggcctgattttcgccatttgattcatgctttcccgcccccggctaaccatgaaccccaaacgtactacgcttgctgctatatctccggatggtttcgtgatttatactaccatatccgtaaagctcttaagagcctttctggactggctgaactcgaatattatcgtgacccaatccctcaaatgtcatttgaatatgacaatttcctagctctactaggtgctgcaatccgcccgactcacattgtcggaaacccggaagacgttatgtacgtccccctcatctctgaagccactgatgtgaaccaagttaatccatttgggatcaacaactatgtccatcaacctgagctcttctctgccatcgtcgctacgatgaaagataggaagaagttcaacatggtccccctaccctctaactactcgggacgaccttcttggctctttgactggcacgctgaagatcaaatctgtgccccctttcaacccgaagggaattttaacaatgaagacattgctatggctttcgtcttagggaccgcctgcacttccttaatcggaatgagagaccgtgatgattggcaattctatccaaatgccaacgttccagctcacttcaatcctcatactgctcagagaatcactcctcgcactatgtacggatcttatgaagtcaggtcttatgaaacagatcatgattactacatcccgtcaaactcggcaggagtaaccgctttcctctcaggaccctccacgtccaggaaaaggtctcgtaccaccagctcctctaccccaggctcactggtcatctgtaagtatatctgtttgtatccataccgattacattacctccctgttcatattaacatctcactatttaattag

SEQ ID NO: 350 - DNA, 2022 bp, Primula malacoides virus 1
  source 1..2022: mol_type = other DNA
atgtctgataccgtccttcaagctaagactaacttccttaagaataagtcacctgtccttagtgtccaagaaagcaaacgacctaaaatctcacgcgacctaatgccacttttccaaagtaacactgctgctcgctctgactcatccaaccaatggactattgatgtctttccaaacatggttcctattactatgtatatcatgctcacctcaattgctcacgccaatgatgttgaccaccgcactacctccaaagtgtccgttgcaactatttgcatgtaccatctctccatcgtctacggttatttccttctcaatgacatgattactcgtgcctccgcttctgctcatgcccgcccctggattgaaacttcctggaaacgcgaattcgccgactttttgctcacactccccgtccctgagtttcttacgcctttcctttctcaattccaacactttactactgatcgtactaagaatgtcttccttacaccttctgctgctggctttgaccataatatattctttggccgtgtctttcctctcaacatgttttctgcaatccatgattgcaccgctaccctgcctggtaatactccacgcatccaagttttacaagacttatatagtcgtgttctgtatacaattactgctcctggcttcacttgcttaatccctgatttaattggtgtcacaatcgatcaaaccactccgactactgcaaactatatgaattccaagctgtaccaagtctacaccgccgtctttaatcctgtcttatttcgcgatttccaacatcgttcatcacttgctgctctttcattccagtctcctgtgtttgctaacaatcacgtcaacgcctatgatttactgttttctgcttccgccgctaatttacgcgaattgaaagttgttctacaagttgtctctaccgtccttgacggaaaagttaccacatccggtacccttggtgatttcctcacgaaaccttctggtgccttagctataaaacatggttattcaacctatgctctaccaacctggtcatacaacactaacgcaacaaaatctgctgtctatagtgctattacgactttgactctcacttccgaacgagatagagcccaagacatttgtttcctccaacgccctgctgctgcatttaatgctacaactgaagttactgatgtaacttatgctgctactgctactccaaatcaagctgttcagttaccaactggtcaccaacttacaaggcacttccccttttccttgctccaagttccaaatggtgcggaaccttttccgcgccatgataatgatgacttagtccagttttctgactttgtgcacactgctccaagcgttctcgtcctcgatactgatggtgaattaaccatctctgctcacttgccactccttgctggcaaaattattgaatctttcgaaattgatggttctaccatcgaaatgccgaatgttgataagtctctcggcctacaaaactgtctgtttgctgactctgctgttagttatagactagtccgccctggctccgcttttcacccgcaacccgctggatcaataaacccccctcttaatcgagttcgccccaacccccgtccccgcttacctgcgtcaagcctgctctatgatcgtactgaaatcatgctcccgaacatgaacaatcgtatagttgaagctcgtgcccctgataccttacccggtatgactagacgtgctgccgtaaactttttacgttatgcgcaatctttcttcggttttcacaccgtagattctagttcaaatgctctttctcttgacaccattcccggaatgacgaatggtctgttacacctctggtctccatactcttacactccgtttgagaatgactactttccagacccagacctcgctgaatcaaggcactacttcttgtccaatctgcgtaccatttttggtactgactacaatcttgttcaagccaagcacccttatgaagctctccctgttgtttag

SEQ ID NO: 351 - DNA, 1014 bp, Fig cryptic virus
  source 1..1014: mol_type = other DNA
atggcagcacccaatttcgacattcttgaagacctcattgctgatgggcgcctccaaattaccccttatgtcacctcaactgaacccaaaatatttgacgtcgatttcgacgcactctggaacaggattaacgctattatagttccaatttacactaattgggatcaactcaggtttcattatcccggaacaactactactgtgacaaacaacatcacccttgacgttggacacctagtcacatttgcgctctggcaactttataacagactccgatctgttgctgaatgcttccaccacccaacgcgtacctcgcgaatgaacacacgagctccaatttctaacaggatggagtttccaagctttctttcttctatgcttgaatctattagctggctccgtattaacgacggacctgttgattatcttgctttatttactgcacctacaggtacatcaaacaactacggaagaagcaccgttcaagtcccagacaatggctgttacgaccgcctaactggaaagctgcgcgcacttggagtacagcttactccaattgatatgacgccatccgctggctcattttggccgactactcaagtcataactgaggaggagctatacaacatcgttggcacgtttcactcatctcattacgtcaatgaagacgcaataagagcaatcttcctcgcagggacggcacactcacagccatttcctgactttggaatgactgttggattcgtcaccgacagaacagtcatggacccacttgcggcactagctgcaccaactggcactccaagatctgaagaagcaacatcggcaggcagacctgttcatggcatgtacaatgtcaaccaccgcggcatacaaccagctgcaggtgaacagaacccctcaggctgctacatcattggtcgtggctatgaacgccgctacacatgctacctcgcacgccgaatcgctgttttcgaggcgaaccgcattatccgttatcgctttatgcgttaa

SEQ ID NO: 352 - DNA, 1248 bp, unidentified
  misc_feature 1..1248: note = unclassified Partitiviridae
  source 1..1248: mol_type = other DNA
atggcagaccaaggtaataatgagactcctctaccagtccaggcaacccaaccagccgcaaacagcgatcgtataccgatcacgaccctccctgcccccaggtcacaagcccccaataaacctgaatacgcacggattgcagactccctggaagacgaatatcaaatcggattccaacggatcaatccacgacttcaccgtcgttacgttatcctcaggtcacaagagctccacgaccgattgtgcaacttgtatgcaccttattttcaagctggatatcgtgttttcaaacacgttatcgaagcttacccactaccagcgggatacgctagtcaaatagcatatgccactgcgtgttacgtgtcttcgtggtgctgggatctttacgtctcgatccgagaatcagttaagaagctatcacaaacagcgttcctccaacaataccaaacagaacaattccacagccaggaccgctacgacccattcctccagcatcttaacaccgtcatccgacctacacacatccccggtggtttagaagattcactctatatcccacttattgctaaccacgtgcaatggggaaaccaaagcccattcggcattaaccattatgccataaatgaatctctggtacaaggtctccttgacgttatggaccttaagaacaatacttggcgtactgttcctctgtcccatgacacactaggccgcccaatgtggcttctagattggcgtcaaaatcaagcttacgcctggtttccacaagaaaaccacttctctaaagaagatctaatcgccgcccatattcttggaatcccctgttctccaagactaggccctcgcgacgttgatgattggcaattctttccaggaaatgtcatgccacaacatatcaatatcgccgcttatgaaagagcaattcctcgacgattcttcggagcagcagagtatcggacgatttccactcgcatgtgggaacttcccttcctatcagctgtcgccggacagatgtcaggagcaaagcgcccagctccagaaagctcggccaccggaagcggatacatggaatcacctccagccgttgaaggtcaacagacagcagcaattatggatcctggaataatgcaaacgcgttattgtcagtatcaaatcatcgattggtgttatcatggaagagtaatcctcaacctgaacgctcagaagcaaaatcaagctctccgctcaattttgttctctccaaattga

SEQ ID NO: 353 - DNA, 2022 bp, White clover cryptic virus 2
  source 1..2022: mol_type = other DNA
atgtctcctgatgagaaccccactctcaacaagacccctaagaaccaaactgttgaactcgtttttcaagataccaaagttcccgttccctctgataacttcatggaatcaatccgctctaacgctgctgctcacactgacactgctaatcagtggatcattgatgttttcccaaacatgactccaatacttctgtacgttatgcattatactcacttatatgctcaagctactgaatatcgcattcatgccaaatcatctgtttacactttgtgtatgtatttcatgactattgtctatggatacttcttgctgaatgatatctacacccgccctgcgcggtctgttcatgcccgtatttggattgaatcttcctggagatctgaatttgtcaaatttctgacgactcttcccgtcccagatattcttaaacccattctgtctcagttccatcctttcactactgctagaactaaaaatgtgttcttttgcccctctgctgctggatttgatcatgaccaattttttggccgtacttatcctatgaatatgttcgctgctattcacgactgcactgctacacttcctggtaactctaccagaactgatgtttttcgtgatttgtactcaagaattctgtattctataactactcctgcttatacttgcgttattcctgacttgattggtgctacccccgatcaaacaacccgaacaacgataaactttatgaactcaaggctctaccaaacctttaactctctgtttaatcctgttcttttccgcgactcccagcgccgtgctgctcttgctgccctctcatttactgctcctacttacccgactaaccatatcaatgcatatgattacctgttctctgcaaccgccccaaatcttagagaactaaagattgttatacaatctgtgattgcatgctccaaaggtgtttttgatgtttccggaacactcggatcttatgtttctgattattcctcttcctcaattgtccatcatggttactctacgtatccactccctgtctggtctcataccgatgatgccaccaaaccgcaaaccttcactgctgccacaattttccaactggtcactgaagatgccagagctcaagatatctgcttcctccaacgccctgctgctgagattgcccacactcgtcgtatcactgatttgacttatgcttcaactgctactcccactgttccagctacaatgcctgcaaatcatggtatatttcgaatttggccctactgtctccgctgtgacaatgatgccgaaaatggcttccctcgtcacgacaatgaagatctcgtcgcttactctgatgagattcacactgctccaagcgttcttgtcctcgatacccctggtgatactgttattagtgcccatatgcctaccgttgtcggtaaggtaattgagtcttttgaactcgatggctctacaatcgcaatgcctgatgctagaaaatcacttgccatgcaaaactgcatgtttgctgactctgccattgcttacaagtatgttcgtcctggatcctactggcgccctcgtgctgctggttccatcctgccacctcttaatcgagccctccctactgctcgcccccgtttaccagcttcctccttgcttcatgacagaactaaggttttcctacctcagttgaatgctcatgttaacgctattcctgaaaataatctgcttcctggatttactcagttgactcctgttaatgtcattcgctatgtccagcccttcttcggttttcacactgttgatcgtactgctaatgatgctgctcttgatgctgttcccggtatggctgaaggccatttgatgatctggtccccatacacttacaatccgtatgagtctgatgattatcctcccgtcgcccttgatgcgtctagacactactacctgactaatctgcgtaccatttttggtactgactacaatcttgttagtgtcaaacacatctatgaatcctaccccgctgtctaa

SEQ ID NO: 354 - DNA, 2022 bp, Red clover cryptic virus 2
  source 1..2022: mol_type = other DNA
atgtctactgaagagacccttcctcctactaagactattaagaaccaaaatgttaacctcattttccaagacaccaaagttcctatcccttctgataactttatggaattgctccgctctaatgctgctgctcacactgatacctcaaatctatgggttattgatgtactccctaactttaccccaattctgatgtttattatgcattacgctcatatccatgctcaagctgccgattatcgcgttcatgctaagtcatctgcttacactttgtgtatgtactatatgactgtcgtctatggctacttcttgctcaatgatctacacactcgtcctacccggtctgttcatgcccgtgtttgggttgaatcatcctggagatctgattttgttaagttcctgactacccttcctgtccctgaatttctcaagccaatcttgtcccaatttcatccatttaacacagacagaactaaaaatgtgttctttagcccctctgctgctggatttgatcatgaccaattctttggccgtatctttcctatgaatatgtttgctgcattccacgattgtactgctacactccctggtaactcctccaaagttgatgtccttcgtaatttatacacccgtaatctatacaccataactgctcctggttatacttgtatgattcctgatttgattggaattactcccgatcaagccgctgccactactgttaactatatgaattctaagttttatcaaactttcacttctatctttaatcctgtccttttccgcgactcccagcgtcgagcttcacttgctgctctctcattcactgctcccacttatccgactaaccatatcaatgcttatgattacatgttctccgctacagcccctaacctacgcgaacttcgaattgtgttacaatcaatcattgctattaccaaagatgttttcaaatctgatcaaacgcttggtaattatgttgctgattactcctccggctccataatccatcatggttactccacttatgctcttccaacttggtcacatactcctactactggaaaatttgaccgatttaatactgctaacaccttcaatttagttactgaagatgctagagctgaagatatctgtttccttcaacgccctgctgctgagatccctcatacccgcctcatcactgacatagtttatgctgctactactgctcctgctacttccttgactgtacctgctaaccatgctctctaccgccgttggccttactgcctccgccttgatgaagatgctgctaatggcttccctcgccatgataatgaagatcttgttgctttctctgctgaaagccatgctgctccacgcgttcttgtcctcgatactcctggtgataccgtgactagtgcccaccttcctactcttacaggtaaggtaattgagtcttttgaactcgatggctctactattgaaatgcccgatgctagaaagtcccttggcatgcaaaactgcatgtttgccgactctgccatcgcttacaaatatgtccgtcccggatcttactggcgccctcgtgctgctggtggaactctcccaccgttgaatcgtgcccctccaaactctcgtcctcgtttgccagcctcctcactgctacatgatagaactaaagtttttcttccccagttaaaccgacacgttaatgccgttcctgataatgatgcccttcctggtttcactcagattactcctgttaatgtcattcgctatatccagtcttttcttggatttcgcactgttgatagttctgccaatgaagctgcccttgatgctgtccctggaatgaatgaaagcttgctgatgatctggtccccatatacctacaacccatatgagtctgatgattatcctgctcttgcttatgatgcctctaggcattattacttgaccaacctccgtaccatctttggtactgattacaatttggttgccaccaagcacatttatgagtccttccccgctgtttaa

SEQ ID NO: 355 - DNA, 2022 bp, Hop trefoil cryptic virus 2
  source 1..2022: mol_type = other DNA
atgtctactgaagaaactgctcctcagactaaggcaatcaagaaccaaaatgttgtcctctcctttgaagatggaaaaccacctggtcccgtctctgacttcatgtctttgttccgctcaaactcttgtgcacacactgatactgctaatcaatggatcattgatgttttccctaacatgactcccattctcatgtacgtcatgaccaaagcccaacaccattctgaagctactgactatcgtctccactccaagtcatctgtcttcaccatctgtatgtactacatgtccatcatttatggctacttcttgttgaatgatttacattcccgccccactcaatctgcccatgctcgcaactgggttgagaactcttggaaatcctcctttgctaagttccttatgacacttcccgtgcctgaatttctgcttcccatccttgctcagttccacccattcaataccgacaggactaagaatgttttctttagtccctccgctgctggatttgaccatgaccaattctttggccgtgtctatcctttgaacatgtttgctgtaatccatgactgcactgctaccctccctggtaattctaccagaacccaagtcctacaagaccttttctcaaaggtcctatacactgttaacgcccctggtttcacttgtctgattcctgatctatttggtcttaccctcgatcaaacagttaacaccactgttaattacatgaattccagactttaccaagtcttttcatcactgtttaatcccgtgttattccgcgactctcagcgccgctcatcccttgctgcactctctttcaccccgcctacatatacaaccaatcatattaatgcatatgactacatgtttgctgcctatcctgcaaatctccgtgaattgcaagttgtcctccaatccgtctctgccattattaaagatgttgctgaatgtaagatacccctcggtacctatgttgctgattattcttctccatccattatccaccatggatactcctcctatgctctcccaacttggtcccattctgagaccgaacaaaaagttgaccgatttgaaaatgctaccactttcaatctcgtcactgaagatgcaagagcccaagatatctgttttctccaacgccccactcaagctatcccacatgaacacaatgtgactgatctcatattcaccgctgctggcgctcctgataccgacattcccctgcctgctaatcatgcaatcgttcgtcgatttccgttctgcctccgccttgatgcccctgctgaaaatggcttccctcgccatgacaatgaagatttagttaagttttctgatgatacccacagtgcccctggcgttctagtcctcgataccatcggtgatggtgtactctctgctcacctcccgactctcactggaaaaataatcgagtcatttgaactcgatggttccacaatcgaaatgcctgatgtaaggaaatcactcggcatgcaaaactgcatgttcgctgactctgccgtcgcatataaatatgtgcgccctggctcctactggcaccctcgcgccgctggatctactcttcctccactgaatcgagctccacccaattctcgtccccgtctcccggcttcgtctctgctccatgatagaaccaaagttatgctgcctcaactcaatgttcggattaatgctgctctcaatgacaatgctctccctggtcttacccgtgttacgcctgtcaatgttgtccgctacacccaatctttccttggattccgcacagttgatagttccaataatgctgcaaacctcgatgctgtccccgggatgactgaaggtcttctgatgctctggtctccctacacatacactccgtatgagtctggtgattatccattcgctgatctcagtgcttctaggcactattatctgaccaacctccgtaccatctttggtactgactacaacttggtttcagttaagcatccctatgaagctttccctgtcgtttaa

SEQ ID NO: 356 - DNA, 2022 bp, Dill cryptic virus 2
  misc_feature 1117: note = n is a, c, g, or t
  source 1..2022: mol_type = other DNA
atgtcttctacatccctcacatcccgtctcaatgccctcaagactaagacaatgaacctcacctttactgatgtcaaagcccctgtaccctctgctgatatccttgaactcttccgtgccaacactggtgctagagccgattcctgcaatgactggacaatcgatgttttcccaaacatgactccaatcatgatgtacactatgctgatgtccattcgacatgccaatgctactcaacagcgcgaacattccaaagcttccgttgctactatctgcatgcatcacatggccgttatctatggcttcttcctcgtcaatgaccttcatgtccgtcctgctccgtctgctcacgcccgctcttgggctgaaacctcctggaaagatgactttgtcaattttcttatgaacgtccctgttcccgagtttctgactaccattctctcccagttccatgcctttgaaactgacagaacaaagaatgttttctttgttccatctgctgctggttatgatcacgaccaattctttggacgtgtttaccctctgaatatgtttgcaacaatccatgactgcactgctaacctacctggcaactcctcaaaaattgatgttctcaaagacctttactcaaaagttctttattccattcaagtgcccaactttacctgctgtattcccgatcttatcggtgttacaattagtcaagtgactaacactactgccaaccatctgaattcaaaactcttccaagtgtttaattcattgataaaccctgtgctgttccgcgacttccaacgccgctccactcttgctgctctctcattcaagtccccaacataccaaactgatgatatcaacgcctatgacctgctcttctctgcaactcctgcgaatctacgcgagttgaaagttgttatgcaagctgtctctgccgtgctagctgattctgtcccgtgtaaatctacacttggacgttttattgccgaaaactcatcaagtgccataatcaaacatggctactcaacgtatgctctaccaacctggtcacacaacgagaactctaataagtcctcgctatttgctgctgttaccacacacaatctcgtatctgaagaanaacgagctgaagacttctgtttcctacaacgcccgtctcaagaaattcctcatactcatgagctgaatgatctgcgatacgtcgccgatactgacaaaagtactgctgttgcgctccctgccaatcatagtattattcgtacttttcctctctgcctccgctacaattccagtcagaaggatggctttcctcgccacaacaataatgatctcatcaagttccaagatgaaatccatacttacccaagcgttttagtgctcgatactgatggtgaccttactgtcactgctcatctgcccctacttgctggaaagattattgaatctttcgaaattgatggtacaacaattgaaatgcccaacactttcaagtctctaggcatgcaaaactgcatgtttgctgattctgccatcccttacaagtatgtcctgcccggatcctactatcatcctcgcactactggcactgttctccctcccttgaaccgagctcccccgaactccaagcctcgtctgcccgcatctaccttgctccatgaccgcctcaaaatcatgctccctcgatttaatgtcgcaattgctgaacaaaacattggaaatacccttccaggtatgactccaattgctcctgtcaatgtgctccgttacattcagtcctttttaggatttagaactgttgatgcctcctccaatgctgctactcttgatgctgtcccgtcgatgcccactgatcgcctaatggtttggtctccctacacatacaccccatatgaacctgatgactatcccattcctgatcttggcgcttcaaggcactactatttgaccaatcttcgtaccattttcggtactgactacaaccttgttcaaaccaagcacccttatgaagctctccccgtcgtgtaa

SEQ ID NO: 357 - DNA, 1473 bp, unidentified
  misc_feature 1..1473: note = unclassified Partitiviridae
  source 1..1473: mol_type = other DNA
atggaccccaacgtccctattgctcaacccaatggccccgctatcccaaacgttgccgctgccgctgctaaccctcctccaccgattcaaccggcccctcccgccccaggtgctgtccaacaagcccctgcccctgctccccgccgcaatcgcttaccccacggtcctgtgcccacatcttcccgcaccgccgtaacctccgctcctgctttgctcgagctcgctgccgcaactcctatgtacacggaacaacgccgtgctacaaacttctttgtccctgactctcagatgctcttccatgtgctgagtatttgtgaccaaatgatgaactctactgaacgatttcttcgttcttctcctgcctggatgcccattgtctcacagctctacatttcagttctctggaatgtcatgattctcaatgtttttgtcaactctggctatggctccgccttttcttcgttcctatctgaactttatggcgtcctccggatcgacgaatgcatgatccccggaccactcgtccccttcttccaagctttggccgccgtcaatggcccctttgactggattggagacatcgtccccggtctccccaacttctctgcgctttggaatgaagctggtttctatgccaacagcaattatgctaggcaagtacccatccccgcccttatcttggaccaactccactacttctcacagtatgccatccctgctaaccagcagtcaacttacgctactttcgaatggtacaggaacgttttccaacaaggcctcggtgccatgaaccgcctcaatcgaattggcccacaactttgtggctccctattcaccacccaagctgagtacgatgccgcccgcaacttctggaatgctagtctgaacactggtatcaccagagctaatgccgcagaaggccaacccgccttcaacaactatcgccaactttttggttttgaatcccaattaggcgccctccaactgaactggttcactagtgtctctatcgctatgaacaagtatactcagttcttcaacggctccgtccctctcaaatccatcctcccaaccggaattggcgctgttgtcatcgctggctcacctgtcaacaatcctagtactcgtacttggatttaccctcctgacacctcaatcgagccatttacaactacacgctacccaccacgccgtgaaatacccgacgctatggccatacaatttgctcattcagaccatgaattagaagagcaagctgaacaatatgccatgctcacccacaccaacatcaaatggcatgcgaacctcaccgcccagaacaactggactgccatcactgctgacgggctctatcttggtgattattggaacatgatgccttacaggttttttgctccacttcactacaaggcccaatttgcccaaatcatcgcctctcgctaccaccaacaggccgcgaaccgcgccgaatag

SEQ ID NO: 358 - DNA, 1464 bp, unidentified
  misc_feature 1..1464: note = unclassified Partitiviridae
  source 1..1464: mol_type = other DNA
atgaatcacaacactcctcctgctgacgccaatggccctgcccttcctgaaggctccgtccctccccctaaccctcctactcacatccctgtagcccctgctgctcctggcgccgtccaaacgccgcctgctccccctgcccgtcgcaatcgccaaccccatggccctgtgcccgctgccagcaaccaccatgctcctggagctcctgctctcctcgaactctccgctgcctacccgatgtatactgaacaacgtcgtggtgttaacttctttgtccctgactcccaaatgctctttcacgttttgggtatgtgtgaccaaatgatgaactctactgaccgatttcttaggtcctcccctgcttggctccctatcgtctcccagctttacatagctgttctctggaatgttatgatctgccgtgtttacataaacactggctatgctagccatctagctcccatcatcgatgttctcatctctcatctgcaaatccatgaatgcatgatccctggacctttagttcctttctttcaatcccttgctgccgtaaatggcccttttgactggataggtgacattactcctgccatgcctgcttttgcaagtctctggaatgctgaacaatttaccgctcgagatagttatgctcgtcaaatccccatacctgccattattctcgatcaacttcaccactttgccacccgtgctattgtagccggagttgccaattatggtacttttgaatggtatagaagcattttcaaccgtggtgttggtgcaacccccgccaactcaagacttggaccacaactttgtggctcccttttcaccactaatgctcaattcgatgccgctcgtgctttttggaactctgcccttgcttctggtattacccgtactgatgccgctcgtcccccgctcgtcaattacgctcagttgcttggcttccaagcccaagatggttcagaccaactcaattggtttcagtttacttcttcagtgatgcaaacttactgccaatacttcaatggttctgtgcctctcaaatctatctccctcactggtataggtgctgtcgttacctacggaatccccgttaacaacgtagccacccgaaactggttttatcctactgctgctatgatcgaacctttcgccacatcccgctatgctccccgccgtgaaataccaaatcaacttgctgtcaaatttcaacatgctgaccacgaggtcgaagaacaagccgaacaatatgcgattctcacccacactaacattaagtggtacgaatcgcttatcacccagaatgcgttaactgccgtcaccggtaacctcgtccaccaaggcgactactggcgtatgatgccgttccgctattctagtcccatcagccttaaggtgcaatatgctcaaattattgcctctcgctaccaccagcttgctgccaaccgtgctgaatag

SEQ ID NO: 359 - DNA, 1185 bp, unidentified
  misc_feature 1..1185: note = unclassified Partitiviridae
  source 1..1185: mol_type = other DNA
atgacaactggtgatcgtgtaacacctactgacggcattacaaccgtcgatgcacccacagcaactctcgaggctaagcttgactcttcgtctaccgcttacctcaaaagcgcgaccgactttctcactcagagtagtcggattggtaccttgaggaccagctacaaggaccgccgtagatacgtctacttaaagtcccaagctatgtttgactcccttgtcgagttatactctcttctctttaccacccagtggcaagttttccgcaggtttactgatattttcgttcctcctgccaacgttactgccacccgctacctagccatgaactacatctctatgtggattcaggatacgtatgcgatgaaccgcgctgccctcatgaaattatcggtggtagctttaaatgatcgctttcctgttgagatacccattgttggttatcaatacgacgcgtacctcgctcacctgagcgccgctctccgcccaaccctcaccaaacacatgatggaagacactctctggatcccctgcttcacaaggtcgcccaacttccaagttgaaagccccttcgggattaccggttacgtcatcaacgacgtcatctttaccggagcgatgaacatcatgaaagaacgcaagctttggaggatcattgatctcccaaccaacgttctcggccgtcccagctggcttttcgactggcatgacgatgatcgagtctgtgcttggttcccaatggaagggaatttcacaatggatgatgtcactatggcatatatccttggagtcgcctgtactcctaaactcgccccatgcgatgttgatgattggcaatactttcccaacgatgtggtcccagccaacttggacccctatgcctttctccgtctgacaaacagacgtttccatggaggttacgagattcgcaccctcgacgcagctaacatgcccggccagaccggaacaacaactgacagtcgtaagcgcccacgtcccggcgagtcaacctcagcccctgctctcccgcagacatcggagccgcaaacgcaaatgcaagtcgacactcccccagctcccaagccaagattccgtctcgtagactggacctaccactacttggtcgttctcaacaaggaggatcacactcgcttcgctgccctcaaaagcatctgctggaactga

SEQ ID NO: 360 - DNA, 1464 bp, unidentified
  misc_feature 1..1464: note = unclassified Partitiviridae
  source 1..1464: mol_type = other DNA
atgtcgatgaaaagaaagtcaaggcctagttcttcaacaaaagatttagaggtagaagacacctatctcaaacaatctggacttgattcaatgaataagctcgaaccagtcgagcaatcaaaagacgaagagactaccaaagtctccatgctccccaccgcttctactgctataatcgccccgcgtaaactaactgcggaagattttagctctaaacgtaagccggatcaaacgtccgctgttagcccattctttgggttccttaggacccacatcctacaccctacacagggcaggctctcacactactacccttcctgccacatgatggactacattcttcactctatcaattcaactctctgtgataattactacttcaagagagaaactccaaactaccacccttacattctccgactctacttcggagttcttttctgggttcagtgcttgcgcgctggaaatgatgttcaggtcattaatgacctacactacgatttcttgcagcgtttcctagactgcaatcctctcgagtctttggccattccaggcccacttctcggactcttcaagacactctgctcctcccagccagagttcccacattatgggaaagtttacccccgcattcctgcgagcccaggaccaagacgccgagacatgttctcaaagaacgtcccaagcgcccaattcttacccaacgttcctggtattttcgcactcatccaccatctccacggactttctgagggtgaacacccgatttacccgaaaagaaaaagacacattcctgtcactgaggaggccagtaacttcggtttcaaggccttcgccgcttttccaaacagaatccagcgcgatcgttggatggttagctcccccggcctccagtatccctgtgaagccgacatgaagatgaatgaagcgttcgctgaacgtttctatgattttgactttcctgcctttaacgcagatgacaatctctccaccatcaccaacttcctccacatgaggaaaagtatggcttggttcatccgggccaaggaagtcgcctgctcggccgctagattcttttcagactctggcactctcgccgactgttctccacacggtctggtctcaaaccagatcattgttgcgattactcctccacctgaggagacttttgctgatccccgcttctccgccgatccaagagccctctatcccttcagtttcaagctgaagagcaccgcccacaacctccccccacttgcggaagctgccgcagccttctcccagacgcacatccggatttttccggaatacccgttggccggaaactttggtcaaaagaccgacgaatcaggccccttttgggacatcaggcccattggctccagccccaccgacgacacctcctacctcaccatcccacccatggtcaaggcagcactcatcgagaaaggctccagccgttag

SEQ ID NO: 361 - DNA, 2019 bp, Cannabis cryptic virus
  source 1..2019: mol_type = other DNA
atgtcttcctcaaccatcacttcccgtctcgatgccctcaaaagcaagaacgtgataattgaattcaaagaagcgaaaactcctgtcccctctgctgacattttgcaactctttgctgacaacaccgccgcccgctctgactcatccaatgaatggattattgatgttttcccaaacttcactcccatcctgatgtacattatgctcaatgcgattcgccacgcgaacgccactgctcaacgtgaacattccaagtcatctgttgccaccatttgcatgtatcatctgtccattgtctatggatttttcctcatcaatgacctcaatgtccgtcctgctccgtctgctcatgcccgtatctgggccgaatccaactggagatctgaatttgtgaaatttctgtccaccctgcctgtacctgagttccttattcccatcctatcccagtttcacgcctttgaaaccgcaagaacaaagaatgttttctttattccctctgccgccggctatgaccacgaccaattcttcggacgtgtttatcctttgaatatgttcgccgcaattcatgactgtactgccacactgcccggaaactcctccaaaatcgatgtccttagagacctctactccagagtactctactcaattactgacccagcctacacctgcgtcattcccgatcttattggaatcacccttagcgctgctgacattacccaagctcaatacatgaattcaaaactttaccaagttttctcttctgttattaatcctgtcctgttccgcgactttcagcgccgatcctccctcgccgctctatcattcaaagctccaacgtttgcctctgctaacgttaacgcttatgatctgatgttttcagccacttccgctaatctgcgtgaattaaaggttgtcctacaatcagtccatgccgttatctctgatgatgtatcatgtaagaccagccttggtcaatttattgctgatgctaccacctcaacaatcattaaacatggatactccacatacgctcttcccacctggtcccacagtgaagtcaccaacaaagctcaaacctttcgtgccattactgcccaacaactaatctctgaagatgcccgtgcccaagacttctgtttccttcaacgccctgcagctgaaattcctcacacccacaacctcgaagacgtacgttacgtacgcaccgatgcccccactgaaccagtcgctctgccccagaatcacagtatcatccgtaacttccctctctgcctcagaaacaatgcgaatgccgccgtctttccccgcgtagacaacaacacactgatcaattttagtgaagaaatccacactgttccaagcgtcttagtgctcgacaccgatggtgatcaaaccatcactgcccacctgccactacttgccggaaagatcattgaatcttttgaactcgatggaaccactatcgaaatgcccaatgccatgaaatcactcggcatgcaaaactgcatgttcgctgactctgccattccgtacaagtatgttcgccctggctccgcttaccatcccgttaatgctggaaccatactgccgccccgttctcgagctactcccaattccactcctcgcctgcctgcctcgtcgttcctatacaatcgactgcgtgttaatctcccccgattcaacctcgctatcaatgaagaacgcattggcaacattctcccaggaatgactctacttgatggagtcactgtcctccggtatgttcagtcctttctaggattcaacaccgtcagccgtgctgacaatgccgataatggagacaatgtcccggcgatgccccggaacctgcttctggtctggtccccgtacacgtacactccgtatgaatctgatgacctgcctgaaccagcacttggcgaatcaaggcactactacttgacaaacctccgtaccatcttcggaacggactacaaccttgttcaagccaagcatccctatgaatcgctccccgtcgtttaa

SEQ ID NO: 362 - DNA, 1467 bp, unidentified
  misc_feature 1..1467: note = unclassified Partitiviridae
  source 1..1467: mol_type = other DNA
atgaatcctgaaacaccacttgccgaccccaacggtcccaccccacctcacggtgctgctgcccaacccacccctggccaacacatccctgctggaactccctccactggtgctgtccacttgacaccagccccgcaacccaagcgcaaccgcgcgccccgtggcccgttcgacaatgcctcgagcacctctgctcctggagctcctgctttactggagcttgctgctgcctaccccatgtccacggaacaaaggcgttccccaaacaccttcgtccctgacgcccagatgcttttccatgtcctcagcttatgcgacaacatgatgctgtctaccgaccgtttcacccgctctgctcccgcctggctccccatcgtctcgcagctttacatttccatcctttggttcaccatgattctcaaagtgtatgtcaactcctcctatggagccatgcacgctcacctcctgaatatcttagtcgcccatcttcgtatagatgagtgccttattcctggacctctcgtcccattcttccaatctcttggagccattaacggcccttttgaatggatcggagacatagtgccgatcatccccgaattttctcagctttggaacaccacatcgttctaccccactgactcattcgctcgcatcttccccatccctgctatcctcctcgatcaactgcactactttgcctccatcacagtcaccgaacaagaaaccattttcggtcactttgaatggtacagaaacattttctctctcggccaaggcaccttcaacccccgccttcgcctaggcccgcaactctgtggctccctctatgccaccgaatctcaggtcaccaacgctcgcactttttggagcgctgccctccgcaccgggataacccgcaccaatgccgcagctaatcaacctctttttagctcctaccctcaactccttggtttcgtcgaccagaacgacgctatgcagctggactggttccagcacacagccatagtcatgcagaagtactgtcagtacttcaatggctccgttcccctcaaatcaatcagccccactggcatcggcgctgtcgccgttcacggctaccccaactacagcggtgctgctcgcacctggatgtaccctgctaccaacaccctctcctcattcaagtctagccgcttctccgccctcagagaaatccctgctctcctcaccgttaggttcgagcatgctgatcacgagcttgaagaacaagccgaacagtatgccattgccacacatactaacatgtgctggtctaagaaccttacaacccagaacaactggactcaagtagctgaagccaatgtgcactcaggcacttactggtctatgacccctcatcgggcacacgcctctgtaaggctgaagacacaatacgcccagctcgtggcctcccgttatcaccaacaggctgccaaccgtgtctaa

SEQ ID NO: 363 - DNA, 1293 bp, Pepper cryptic virus 2
  source 1..1293: mol_type = other DNA
atggcgacaccagttagtgacacaccaactactaagtctacacaagacaagactgaccaattaccggcagctacgccccctataacgcaaaagccaccctcctacaaatccgagatttcaagaatcgccgactacctcgccgatgactacgaaattggatacgtaagagaccgtccacccaagcatagaagatatgctgtgttaaggacgcaagctttattcaacaaacttatcgaaatctacggtaagttatttaaacagaactgggaattcctccgacgttcaatcgacggtttcacaaccgcagttggcattcgtgagagacaatgggaaaacgcagcccacgcgtatatctcatgctggttctttgacctttacgtttctgtcagaaacgctgttgaaaaggtctcaggtactgcatttacacagtactatcatgcacacgcagctcacattttgcatgaatatgatgcacttcttgttcacttgaatgctatcattcgccccactcatatcaaactcgcactcgaagattgtatatacataccgaggattgcaacaaatatgaatatcaccaaccctaaccccttcggcctcacagaagctacatgggatagagatgctgactttgcttattcgatcatggatcgtatgaaagcaccgggctccagatggagagttgagcccctagttaccgacactctcggcagaccaggatggttactcgactggcaccaggtaggcgcacaacaacaagcatatgcttggtttccttcagaaggcaactactctatggaggatttgattgcaccacacataattggagaggcacttactccacttatgggcccttgcgacattgatgattggcaatactttccaaatttcatccgtccagctaacgtaccatggcagaacatggaacgcgtatctgcacgccgcttccgcggaagcgctgaagttcgaacctatgctgaagagaaagtaaaccttactcgtttcacatctacaggtcacgaaacttcgaagaaaagaagtaagccgtcctcaaccaccacgacctcaagtcaaggcggatccagggacatggaaacagaagatgaacctcgtgaagtcgtaactgatatcacattatcacaggttacatccactcaacaactacaaactctaatgactacaggagaagcagataggtttaggattatcgactacacttattattgctgtgttattcttaggaataccgctcagaaaagagacggagcaattagaagtttctgttttaagcagtga

SEQ ID NO: 364 - DNA, 1236 bp, unidentified
  misc_feature 1..1236: note = unclassified Partitiviridae
  source 1..1236: mol_type = other DNA
atggcagaaaacgatattccagccgaaggaatcccagcaagacctccgcctgccgctaaccccttcacgggattaaaccgtgatgaactcgaccgcatcgtcgagcaaccagaagaggataaccaaattggtttattccgagataaggctagatcttatcgaagatacgtagacttacgtacctctccgcttagtacaaggttaatcaacttgtactcaaaccttttcaccactcactggaacgtgttcaagcacaccatgtctggagaagcccccgccggattcgcctcaaaagcgatctacctggcaacgatctacgtatccgcatggatatgggatcttcatgtctccatcagagaatctgtgagaaggatctctggtactgctttcaaccagctgtttcagcaagatgcttaccatcaggctgaccgctatgacccatttctgcagcacctcaacaccatcattcgccctacaccaatccatcaggcactggaagacacactctacataccagtcgtctcacgaaacatagattgggccaacgccaacaataacgtgttcggattaccaagtcatgcatatgacgacaacatcctgaacgccatcttagacgttatggacgaccgtcgtaatcaatggtcaacaattcccatctctactaatgtattaggtagacctggctggctattagactacagactgggacaagcatacgcttggttcccgatggaagccaactactctcaagtggaccttatagctccacacatccttggaactccctgcaccccaaggcttggaccgagagataaagatgactggcaagttttcagatacaacgccgcaccaccacccggagtcagactgaacccgcacgactttagaagagcaaccgaacgcagcttctatggttccgccgagtacaggacgatcgagcacgctgtgcacgtatttgactggcaagccaccatcccacctcctgacgcagcgacttcagcagcctcgcaaccaggaaagagaaaacaaggcgattcatccacatcagcaatggcgctaatggaaacagacaaggacccaccgcagcccttcgttccaccaccgcctgtggacctgaacagataccgtctcatagactggtgctaccacgcacgcgtcatcgacggcgaagatcagcagactcgcaaccgcgcactaaggaacttcatattccaagcaggccaccaacctacttag

SEQ ID NO: 365 - DNA, 1239 bp, Pepper cryptic virus 1
  source 1..1239: mol_type = other DNA
atgggcgatcgtgttaacgctcaagacgacgatacagtcgtccctcaccaagcacctcttcaaccagcagctcttcagcaagacctcaccagatctgccgactacttattagacaacgtccgtattggcaaccaccgacaacgctatgataaataccgtcgctatgtccttctcagatccagcgaaatattcactagtctcgtcgccatttacgctcacatcttttctagttattggcaacactttcggaggttcactgatcaatttcaagctccaactggagtccaactccccacttttgtggcacgagtctacatctccacttggcttcatgatctatactgctctattcgagaggccactagaagtatttctccattagcctttaatgagaggtactcatatgaattgctcccatactccaccgagtacgacccctttctcgcatttctgtctatgtctattaaaccaacacatattcagcatactcccgagaacacattatggattcctatcctttgtgaaaattacgactgggatcgaaacgaagcaaatcacaacccttttggcattactaatttcacattgaatagtaacctattctatgggcttttagcgatactgaaagaacggaaggagtttaagttatcaactctgactactaatacaataggccgaccctgttggctctttgattggcacgacaacgtgcaagtctgcgcatggtttccccgtgaagcaaacttcaacagtcaagatgtaaccgccgcttacatcattggagtagcctgtactcccaaactaggacccagcgatgacgacgcatggaaatactatgcatccctcaactctgtaccaactttcacaccaactgagccgcgtctcaccaatcgtcggtcatacggtgcttatgaagtccgcactcgtgaaactgaaaataactatttcttaccagatagtcttctcaacatcatcgaggattttaccgcaacaggtacaacacaacgaaggaagatccgtcgcccttcagctacgtccgcgtctaccggagccgctatcattatcagagatacgcccggaacagcgtccactgcaaccacgtccactacggagaccgaagttaccttccctccagtcatcaggactaagattagagactggtactaccactctcgtgtaattctagaacttgaagacaattctaggacagcagctttaagaatgtttatcattgcttaa

SEQ ID NO: 366 - DNA, 1473 bp, Carrot cryptic virus
  source 1..1473: mol_type = other DNA
atggatgacaatactcctgctgcccaacccaatggcccccacgctcctgctgccgctgccccccacgctgttccccctcctgcaatccctcctgctactcctgcccctggtgctgtccaacaaccacctgctccggctccccgtcgcagtcgcaaccctcatggccccactcccatctctagccgcgtatctgctccttctgcccctgctctcctagaactccctgctgccacccccatgtacaccgaacaacgccgctccgccaacttcttcgtccctgacgcccaaatgcttttccatgtgttaagcatatgtgatcagatgatgaactctactgagcgctttctccgctcttctcctgcctggatgccaattgtctctcagctttacatttcagttctctggaatgtcatgaccatccacgtctttgtcaactctggatatggctctgctttcagttccttcctcacggaactgtacagtgtccttcggattgatgaatgtatgatccctggacctcttgtccctttcttccaagctctaggtgccgttaatggcccttttgactggattggagacatagtccccggactccccaacttctctgcgctttggaatgaagctggctttcacgccaacaacaattatgtccgtcaggttcccatccctgccgtcattcttgaccagcttcactacttttctcagtatgctgtccccgccaaccaacagtctacctacaccacttttcagtggtacagaaatgtgttccaacaaggccttggagccatgaaccgtctcaatcggatcggcccccagctttgtgggtctctcttcaccacccaagcacaatatgacgccgctcgcaacttctggaatgccagtctcaacgctggtattactagagccaatgctaacgaaggccaacctgccttctccaactatcgccaactttttggctttgaatctcagactggtgttctccaagtcaactggttcactagcgtatctatcgctatgaacaagtacacccagtttttcaatggctctgtccccctcaagtccatcctcccaaccgggataggtgctgtcgttatctacggcacaccgatgaacaatcctagtacccgtacttgggtctatccacccgacaccggtattgaaccgttcaccaccacccgctatccgccgcgtcgcgaaatccctgacgccctgtcaattcaattttctcatgctgaccatgagcttgaagaacaggccgaacagtatgctatgctcacccataccaacatcaaatggtaccagaacctggaaacccagaacaactggactgccattgctgctggagggctttaccttggagactactggaacatgatgccttataggttctctgctcctcttcactacaagtctcagtatgcccagatcatcgcctctcgctaccaccaacaagctgccaacaaagccgaatag

SEQ ID NO: 367 - DNA, 2025 bp, Crimson clover cryptic virus 2
  source 1..2025: mol_type = other DNA
atgactaccaactcatcaaacgcccgactcaataagctgaagaataacactgttcctcttcctttcaatgctactgaagtccctgaacccgctactgacctcctcacttctatgaaggagaatacttctgctcactctgctactgctaatcaatggaccatcgatctctttcccaacatgattcctgtcctaatccacgttatgtctgctgcctcacaccatgctaacgcgattaactttcgtgaacactcgaagacctctgctgctactgttgccatgtaccatatggccgtcgtttatggatacttccttttgaatgatctgaacattcgtctcaatccttccgctcacgcccgatcctgggctgaagcctcatggaaatctgaatttgttgaattcctctacggtttgcccgttcctgaattccttgccccaattttatctgaataccagcattttgtcactgacaaaacaccaaatgtccactttacaccatctgctgctggatttgaccatgaccaattttttggacgcgttttccctctgaatatgtttgctgccctgcatgatagtactgctactctacctagtagttctacatatgctcaagtcttacaagacctatttagtagacctttgtacacaatcactgatcctgcctttacctgttacctccctgaccttcttggaataactctcactgctggagaaaatgttaccaccttgaattatattaattcaaaattgtaccaagttttcactactatattcaatcccgttttgttccgcgatcaccaacgtcgctcctcacttgctgcactttcattcaagtcccccacctaccctaatccaaatatcaatgcttatgatttactgttttctgcctctgtcgccaatctgcgcgaaattaagatagttctacagtctatttataaattgttcgatggaagagttgcttgtaaacacactttaagtcagtttatctgcgaatccacttccccgtcaattacaaagcacgggtactccaccttcccgctccctacttggagccacactgaatccgacgcgaaagcaatccgcttttccggcgtcactgcccttgttcatgtgtctgaagaagatagagcccaagatttttgctttttacaacgccctgctgaagctatccctcatgttaatgaagtccccgatatccgctatgccaccactgaagatcctgaaactgctgttcctctccctgcgaaccacatccttgttcgccgctttccattcgcaattcgtttgcgtcaagaagctgcaaatggctttcctcgccatgataatgatgatctcacaaaattttctgaccatgtgcatacagctccccgcgttttaattctcgatactactggtgacggaatcatttccgcccacaccaccactgcttctggcaaaataattgaatccttcgaactagatggatcaacaattgaaatgccaaatgctgacaagagcctaggtatgcaaaactgcctctttgctgattctgccattgcctataaatatgtccgtcctggatccttttaccgtccacgaacacaaggctccgtcctacctccactcaatcgagctccccctaattcaaggcctcgcctccctgcatcttccatgctccatgatcgtactaaggtatttttacctcagttgaacacccgtatcaatgaagctgttgacatccgcacccttcccggtctcactatgctgaataatgtcaattgtatcaactatgctcaatcctttattggatttaaaactgttgatgccactaggaatgatgacgtcctggatgctgtccctggaatgcccctgaataacctgattctctggtctccttatacctacactgcttatgaggatgaaacctatcctgatcccgttttcagtaattcccggcactactacttgaccaacctccgtaccatctttggtactgacaccaatttgatcaaggccgtccacccctatgaagcctaccccgtctcttaa

SEQ ID NO: 368 - DNA, 1281 bp, Beet cryptic virus 2
  source 1..1281: mol_type = other DNA
atgactggacccacacaggcccacctcatcgaagcgacaagtttcacgggactcaaccgtgacgagctcgacagcatcgtggacgccttacaagaggagaaccaaattggtacctttcgtgacaggccaaaagcctacaggcgatatgttcacgtacgctctcaggagctctttgatcggctaagcgaactctatgccgatttatttacaagtaattggtctgctttcaagcatgcctatattacagagccaactggccgtgaagatgcaaacagcatctcaacgaaagcctctttcatggctcaatgttatttgaccgcgtggttctgggacacgcaatactgcatacgcgaaagcgtaagaaagttgtcaggcactgcctacaaccagcatttcacagaggacctcaacctcatctcccctaagtatgatccattcttacaacacctgaacatggtcataagacccactctcatccaccaatccacagaagatacactctatatcccactgcttggacaaacgtttaactacaacgaggatgcttataactttctcaatctccaaggctgcggcaccgaaatccgccaagtttatgccataacagatgttatggacagcagacgcacagaatggtcaacaataccactcgccaccaacgtactgggacgagctagctggctactcgacttcaagcaaggcaacgcctatgcttggtttccgtttgaatccaactttactgagctagacttagtagcaccttacattcttggaataccctgcactccaagactgggaccccgagacgaagaccactaccagcactggacccacaatgatccccctctaagcacagatggcaatatcattttgaatccgctggcatatgtacgttcgtctgaacgcaaattctttggcaatgctgagtacagaacaatggaatataacagctacacacaaaactttgaagcttatatgccaagagttactacctcaacacaagcgaagaacaagcgcaagaaagatgctgcaccagagcagttatctaccggacaaggagatggatcatcatcatcgaaagatgctgagatgtctactgaagaatcagaagctccaccaccgccgaagattccaagaccaagacaaatccatcaattcagattgatcgactgggtttaccactcaagagtaatcctaggagctgacaaagctatgcaacgacgtgccctttgggacttcattcatgctggagcaactccaagtcatcaaccaaggccttag

SEQ ID NO: 369 - DNA, 1182 bp, Beet cryptic virus 2
  source 1..1182: mol_type = other DNA
atggcaccgaatgacgaacttgctgcaaaccttcaaggactcaaacgtcctgctcttgacgaaatagtgcattatcttgaggaccgtaatcaaatcggtatatctcacgataagaacttgtcctacaataggacgtgtgtactactcaccaatgagttacatgccaggctcgtgactctttacacgaacctcttcaccacttcatggcgacactttaaaagtgccgttcgtaaggaaatgccccgcactgaagccaatccacaacgctggctggccaactgctacataactgcatggttttgggatctccaagcctccatccaagaagccaccaagaccttgtctggtaagatttaccaagactacttccgagataacatccaccccatcttggaccgttatgatcctttcctccaacatctcaacaccattatcaagccaactcacattgttaatgcaactgaagatgtcctctacttccccatcatttcagccgactacagacgtgctgatgctgacatgaatatccatcgcattactggtgcattcacccgcccaaatgtggttatggatctagtttcactgatggacgacccaaactccggttggtccacagtacctttaaacacaaatgtgtttggccgcccaggctggctactagactatgacggaaccgatgcttatgcttggtttcctatggagaacaattacaatatgtgcgatctaatcgcccctcacattctcgctacgccctgcacagcgaaattaggcatatatgacgctgatatttggcaaaattgcccaggcaatataccaataacagcaacaacagctagaaccgctcgacgtgaatccgaacgccgtttctacggatctgcagaaaccagaacaattgagcagagaaactacactgtcaattttcctgagcttctaaagccaagagacaccggagcacgggctctagtcaccaccacccagagagcttcaacttctgggtctgttgaaccgacgacaggtgctcaaccagaacctgagacaccaacttttcagataacatttcgacaatttcgcattctcgattactgctatctagcgaaggtcatccacaagtctaattcccagatgatcaacaaggcactccgcaacttcatccagaagggagcctccaagaagaccgagtaa

SEQ ID NO: 370 - DNA, 1476 bp, unidentified
  misc_feature 1..1476: note = unclassified Partitiviridae
  source 1..1476: mol_type = other DNA
atggacaaatctaagaagtatctatctgatgctaaagaaatcatcaaggctaatggtcttgaagaaaaatttctcgccgctgctggtctttcatctatggatgaactcgaccctaagaacttcgctgagtttgaagctcctgctcctcctcaagttatgaagccctctatcgccgctgcccccatcattcaaaaaccgctttccaagcaggttgctgatgcctctactggctttaatccagagagcgcttctgatatgctcagaccatttattggtctaaagattaactacaaagctaggaagactgtatctacctatgctccgtcctctcttatgatggattatattctccatcaagttaacaatttacttgttgataatttctactttcgtcgggcttgccccgattaccacccttacatcttacgcctttactatggcgttttgttttggattcaatgcatgcgtgcaatgaatcatgctaggctgcttcacgctgaagcctcccaatgtctgtcaacattcttggatgccttccctgctgactcccttccaatttcaagtcctttacttgacttgttcaaaactctttgctgctcccaaccagagattcccacttttggagttgtatctcctactctcccattccatcctggccctgaagctaggagcgcttttatgctggctgatgctaccagccatgttttacccaacattcctgggattttcgctttgatcgagaacctccggacctgcctcaaccctggcgatgggcaacagcccaatctgcctgctaagggtaaacatatccccgttaccagtaccgccgctgcggcaactgttttcggtcaccaccaattcccaattttggccaacaggtcaaatgctgaaaaatggagcctttgctcctctggactacaatatccttgtgaagccgatcgtcgacttcacgaaaatttttctgaaaggctcgataacttcgagtttcctgtcactgccgaggatgatgacctcaggttttatagtgactttttgcacattactaatagtctagcttggtttgcccaagttagagatgttgctgccaatgaagctgctttcttcaatggctctggcactttggctgactgtccccctttcgggattgctgccaatcagattactgcgggctatttagccccaattattcaagttgctgctcctacccggagtgccgaccctgcctccctctttccattcgctattaagctcgaaaccgcatctcgagctcttcctgaattagctgaagcgatggctgcacttgcgcagaccaacgtgaatatctacccgacccatccctactacaaccacattggtgctgagtcgcgccgtggaccgttctggaaccttgctcccgttgaatcctccatcaccgatcactcgagctatctctcactccgagaggttgtgcgcggtttaatgaagcccaaaggctaa

SEQ ID NO: 371 - DNA, 1452 bp, unidentified
  misc_feature 1..1452: note = unclassified Partitiviridae
  source 1..1452: mol_type = other DNA
atggcagctttaggtgatggtgttccaccgattgcggacaataacaacccgcaaggaagagatgcaaatcccattaggggaggaactgatcccctacgaagagcaactgactggttggaagacaatagtaaaattactatgacgttccaccgtcatgctaactaccgaaggtatgcaacattgcgccttcaagagatgtttaatgctctggtacaattgtatcagaacatattcatgtcccattggcatcatttacgccggacagttgagaactaccaagttccagctccactggctcagacgccatggctttatgtagcacgtctatacatttctgtatggattgttgatctttacgcctctatacggcgcgcatgcttacgcgtgtgccctgaggcatacaacgagtattatactcaagattatcagcaggtagtctttgaatatgatgcttttctttcacagcttttgtctatgatcaaaccgaccgtggttaacctcgtccatgagaccactttgttcgttccacgactcgcagaagaccaaccgtttcctgccgacgacatgaacatattcggcttagatcgcttcgactacaacgaaaacgttgttcacggagttatctcgatcctccgagatcgtaaattgatgaaatttgaaaagccttcagaaactggattcggacgtcctttctggcttttcgattggcaccgaaccgggcacgcatacgcttggtttccagctgaaaacaactatgatatgcgagacgtaaccgtcgcatacatagtcggagtagcctgtactccaaaattaggaccacgtgatattgatgactggcaacactttccaggaaatatcattcccgaacatcctcgggcagaagatcacgtcagagtgcgccccgtagcgtaccatggagccgtagaagtacggaccatagacgtccaacaatggactgcaccaccagaggtcatgtcgctaattcaagtgaccctgcctccagcagctgcacagcaacaacaacaacagcagcccgcagcccaggcccaacctgctccagcccgccaacagccgcctgcacaacaacaacagcaggaacaaggtttaattagatataacctacggcaaggatttaaacggagaagaactgatccttcaacctccaatgataacgacatgcctgtcgaggacattgaggagccaccacaacctgaacaaggcgaagaccccatccacccggaagaggaagccgccccaccagtagagccgcagcccgatccagctccagcaccagcccctgcaccgcaagcccccgctgagccgatcataacgatgaatcgcgttagaaggtttcgcatcatcacttactgttactacagacaagtgaccgataatgaagacctgcattctcgtctcgctgcattacgacaagtggattcactgattgcatga

SEQ ID NO: 372 - DNA, 92 bp, Pepper cryptic virus 1
  source 1..92: mol_type = other DNA
agaatttcccccttgttccaaggcacctctcacgctccggtgccataaacaagagattgaaattaatcaatttatatacttacctcgttacg

SEQ ID NO: 373 - DNA, 31 bp, Pepper cryptic virus 1
  source 1..31: mol_type = other DNA
ataaatacgtggaaggtatttaaaccttacc

SEQ ID NO: 374 - DNA, 95 bp, Pepper cryptic virus 1
  source 1..95: mol_type = other DNA
agaatttgacctgaaccccatatcaggatcgaattatttacaaattcaccaaacacgtctaagcttgtgaaaattattgttatccctacattaac

SEQ ID NO: 375 - DNA, 178 bp, Pepper cryptic virus 1
  source 1..178: mol_type = other DNA
ttagctttcctttatgcttttgtcatgttataaacaatttcgtttaattttaatattaatcgcagttaattttatacatgtgctctgttatttgatatcagttccgcataatggacaggctacaatacccttcagtagggtcacaaacgtgacccaggaagctgtattgcaccctgct

SEQ ID NO: 376 - DNA, 80 bp, unidentified
  misc_feature 1..80: note = unclassified Partitiviridae
  source 1..80: mol_type = other DNA
acaactcctgctcaagcttttgacaatcaaaatagaacgtcaatctcagttgctctatctcaacttgttacctactctac

SEQ ID NO: 377 - DNA, 35 bp, unidentified
  misc_feature 1..35: note = unclassified Partitiviridae
  source 1..35: mol_type = other DNA
agccgatcgctttgattgtttaaaattaaaaaaaa

SEQ ID NO: 378 - DNA, 110 bp, unidentified
  misc_feature 1..110: note = unclassified Partitiviridae
  source 1..110: mol_type = other DNA
gattaacagccttcataacgctttttgcaatcaaaatagaacgcaaatctcagctgctctaactcaactttttacttactctactgcttacccaagccgcccacaccaca

SEQ ID NO: 379 - DNA, 185 bp, unidentified
  misc_feature 1..185: note = unclassified Partitiviridae
  source 1..185: mol_type = other DNA
gtttggacccagaagtccttaaactctaacccattttgctgtcaaacagtctatctgattagaaagggttttttttgctttaacttaacttttatctttaattttctctttcctatactgattcctcgcttcctcccgaagcgtattcttggaagctcagcttccgacctaggaacaaaaaaaaa

SEQ ID NO: 380 - DNA, 90 bp, Hop trefoil cryptic virus 2
  source 1..90: mol_type = other DNA
agatttttctaaagcggccccgctagaaattaagaattcacattcgtctacattatcaagttttcctgaacctctgctctaatccttaag

SEQ ID NO: 381 - DNA, 147 bp, Hop trefoil cryptic virus 2
  source 1..147: mol_type = other DNA
ttttgtttcgtttttcttctgaactttttagttcagcgttttcaacgttgttttctgtttatgaaaaaaaaaaaaaaaaaataaaaaaaaaaaaaaaaataaaaaaaaaaaaaaaaaaaaaaaaaacaaaaaataaaaaagttccct

SEQ ID NO: 382 - DNA, 109 bp, Hop trefoil cryptic virus 2
  source 1..109: mol_type = other DNA
agattttttataagcggcccccgcctataatatttagaattcacattcgtctaatttttccccttcacaaataactgctaaccccttttattttaactaacgtctcaca

SEQ ID NO: 383 - DNA, 81 bp, Hop trefoil cryptic virus 2
  source 1..81: mol_type = other DNA
tttttttgtgcttaatagcatgaaaatatttctcgctaatttaaaaaaaaaaaaaaaacaaaaaaatcaaaataaatccct

SEQ ID NO: 384 - protein, 746 aa, Hop trefoil cryptic virus 2
MPFNSVRNYLEERSVRVKKEMMTYQSSNRDPDAILEQSQDPDYRRYYDNARYNPSNDLKYRMLSKEYSTLVEAYRLRNDDKHQPYELHQPVPQDAAPVPSYRAPAPGIKPVPLMYHYGHVIHDPVSLTESAIDDDSDTLPDSDEPTVTHFGYPINKRIYDLICNRYPEYLSVISAYCRPIGTVDATFKDFNKEQIPSGPIDPNRKEEVLTHIFRFLDAQPYLPLHFVDTQFCKTPLVTGTGYHNRYSFKQKAHAKYSHPEEYAQMPTSKGYFYNATYENARTLVHYIKEYGLPFNIHYTPEDVDFTEEQIQAYIDSANNFFNDYPTLLFTRNHISKRDGTLKVRPVYAVDDLFIIIELMLTFPLTVQARKQSCCIMYGLETIRGSNHYIERLARSYSTYFSLDWSSYDQRLPRVITDIYYTDFLRRLIVINDGYQPTYEYPTYPDLDEHKMYTRMDNLLTFLHTWYNNMTFVLADGYAYRRTYCGVPSGLYNTQYLDSFGNLFLIIDAMIEFGFRTPEIDDFILLVLGDDNTGMTVISIDRIYDFITFLEIYALTRYNMVLSTTKSVLTTLRSKIETLGYQCNHGSPKRDISKLIAQLCYPENGLKPHTMASRAIGIAYASAGQDYMFHSFCQDVYNMFRLDYKPDARTTLNFQRQIYHNLDDGIPDLATPVVPPFPSIFEVREMYSRYQGPLTYAPKWNFAHFINSPDVTPAHYKTMRMYEIENNITIRPAPTFETVVPTTRNFP

SEQ ID NO: 385 - DNA, 2241 bp, Hop trefoil cryptic virus 2
  source 1..2241: mol_type = other DNA
atgccgttcaactcagttcgtaattatctcgaagaacgctccgttcgtgttaagaaagaaatgatgacatatcagtcatcaaaccgtgaccccgatgcaattcttgagcaatctcaagacccggactatcgtcgttattatgacaatgcccgatataatccttcaaatgatttgaagtatcgtatgctatcaaaggaatactctactctagttgaagcttaccgtctcagaaatgatgacaaacaccaaccgtatgaacttcatcagccagtcccacaagatgctgctcccgttccttcctaccgtgcccctgctcctggaataaaacccgttcccctcatgtaccactatggacatgttattcacgatcctgtctctctcaccgaatcagcaattgatgatgattccgatacccttcctgactccgatgagcccactgttactcactttggatacccaatcaataagcgtatttacgatttaatttgtaatcgctatcctgaatacctaagtgtaattagtgcttattgtcgtcctattggaacagttgatgctacatttaaggattttaataaggaacagatcccatctggacctattgatcccaatcgtaaagaagaagtgctaacccacatctttcgattcttagatgctcagccttacctgccccttcatttcgttgatacccagttttgcaaaacaccacttgtaactggaacaggctaccacaaccgctattctttcaaacagaaagcgcacgctaagtactctcatcctgaggaatacgctcaaatgccaacctcaaaaggctatttctacaatgccacttatgagaatgctcgcactcttgttcattatattaaggagtatggattaccttttaatatccattacactcctgaagatgttgacttcacagaagaacagattcaagcctatattgactctgccaacaacttctttaatgactacccgacgttattgttcacccgcaaccacatctctaaacgagatggaactcttaaagttcgtcccgtttatgctgttgatgacttgtttattataatcgagttgatgttgactttccccctcaccgtccaagctcgaaaacagtcttgttgcattatgtatggacttgaaaccattcgtggttccaaccattacattgaacgcctagctcgttcgtattcgacctacttctcccttgactggtccagttatgaccaacgactccctcgcgtcattactgatatttattacactgacttcctccgaagactcatagtaattaacgatggataccaaccgacctatgaatacccaacctaccctgaccttgatgaacacaaaatgtacacaagaatggataatttacttacctttctccatacctggtacaacaacatgaccttcgttttagctgatggttatgcctaccgcagaacttattgcggcgtaccctctggtctgtacaacactcaatacctcgattcttttggtaatctatttctaattatagatgctatgatcgaattcggtttcagaacccctgagatcgatgatttcattctccttgtccttggagatgataatactggtatgacggtcatttcaattgaccgaatctatgacttcatcacctttcttgaaatttacgcgctaacccgatataatatggtcctttcaacgaccaaatccgtgttaacaacgttacgctctaaaattgagactctcggttaccaatgtaaccacggttctcctaagcgtgatatttccaagctcattgcccagctctgttacccagagaatggactaaagccacatactatggcttcaagagcaattggaattgcgtatgcctctgctggccaagactacatgtttcactctttttgtcaagatgtgtataatatgtttaggttagactataaacctgatgctcgcaccactctgaattttcaacgtcaaatttatcataatttagatgatggaataccagatcttgcaacaccagtcgtgccaccattcccttcgatttttgaagttcgtgaaatgtactcccgttaccaaggtccactgacctatgctcctaaatggaatttcgctcactttatcaattccccggacgtgacacccgcccactacaaaactatgcgcatgtatgaaattgaaaataacattacaattcgacctgcccctacttttgaaacggtcgtgcccaccacaagaaatttcccgtga

SEQ ID NO: 386 - DNA, 64 bp, unidentified
  misc_feature 1..64: note = unclassified Partitiviridae
  source 1..64: mol_type = other DNA
ccgtagttaaagctacggtaagtagcttagtagaaataattatatttttattcgctcccccgct

SEQ ID NO: 387 - DNA, 52 bp, unidentified
  misc_feature 1..52: note = unclassified Partitiviridae
  source 1..52: mol_type = other DNA
tatttgtaatacgttttaaaataataaaagcgctcacgggaggttacgcccc

SEQ ID NO: 388 - DNA, 1389 bp, unidentified
  misc_feature 1..1389: note = unclassified Partitiviridae
  source 1..1389: mol_type = other DNA
atgggtaagagcacacacaacatttctggatacgttttcacagactttcaaggtgaacttgaagacttgcatgagacgcatacagaacgactccaaagggagtcaggcgttacatattatgatgagttcgcgaaagaagaactacttgatatgtatccaaacttatatgatcaatggattcgtgattggtctcggagtttctactcaggagataaacacttagaagccatatataggtatgccaaccaaaatcttcctatcacagctgtgaatcaagaagtctacgcagactcgattgacgcagttcgagatgggttacgtaaccttcacattgtgagggcgcattcggttagaaccgaactagaccaagtctattacatacaatcctcatcagcaggttacggttatcaaggtgcaaaaggtccgttatcagaatacaaccacgacagggccattcgacgtgcaaaagcaacactatattccgcaaccgcagaggacggagagggtattgaccatactattagagagtcagtcccggacgttggctacacgcgcacacaattgacagatctcactgaaaagacgaaggttagaagcgtatggggaagagctttccactatattcttcttgaaggcactagtgcaagaccacttttagaaggggtaatgaacagtgacactttcatccagataggcagtgatcccattgtcaacgtacattgtagcaatttcgacgcaacagtgcatcgatttgagattaacacagcatttgaccaactgaaagagcatatctactttccagatttcgataccgagatggcattcgaactcagtagacaactatttatccacaagaagatagcagctcctgacggtaaaatttactggtcacataaaggaattccatccggaagttactttacgtctttaatcggttcgatcgttaacagactacgcattgagtatctatggagattacaatgtggaagaggtcccatgacatgtaagaccttaggtgatgactcacttataggcgatgacatcttctatccacctgacaagcttcaagaaagagcactcagattaggctggacgattaacgcagaaaagacggaatgctcaacaaatccagcgagcgttacattcctcggaagaacgattcacggcggtcaaaaccaacgtgatctaaagagatgtgtacgtttactgattttaccagagtatcctgtaatttcaggtgcaatatccgcattcagagcacaagcaatagctgaagatgccggtggaaacagccaggtaatcaacgcgattgcgaaacgcttagagcgccgttacggtgtcgcttcggaagaagacgtaccgagttattttagaaggtatcaaatttaa

SEQ ID NO: 389 - DNA, 1029 bp, unidentified
  misc_feature 1..1029: note = unclassified Partitiviridae
  source 1..1029: mol_type = other DNA
acagttaaaactgaactagaccaagtctattatatacaatcctcagcagcaggttacggttatgaaggtgcaaaaggtcctatccacgaatttaatcatttaagggccattagacgtgcaaaggcaacactgtattccgcaacagcagaggacggtgaaggtattgactacgcaattacggagtcagtcccagacgtcggttacacgcgcactcaactgacagatctcactgagaagacgaaagtcagaagcgtatggggaagagctttccactatatccttcttgaaggacttaccgcaagaccactattagaagaggttatgcaaggtaacactttcattcagataggcagtgatcccattgtcaacgtaccacacattttatcagagacaaagaagcaatgcagatggctatattctgtagattggagtaacttcgacgccacagtgcatcgatttgaaatcaacgcagcatttgacctacttaaagagcatatcgagttcccagacttcgatacagagatgacattcgaatttagtcgtcaactctttattcacaagaaaatcgcagctcctgacggtaaaatttactggtcacacaaaggaattccatccggaagttactttacgtcattgatcggttcaattgtcaacagactacgcatcgagtatctatggagactacaatgtggcagaggacccatgtcatgtaagaccttaggtgatgattcacttattggcgatgatatcttctatccacctgacaaactccaagagagagcactcagattaggctggacgattaacgcagataagacggaatgctcaacaaacccagcgagcgtcacattcctcggaagaacagtccacggaggtcaaaatcaacgagacttaaagaggtgtttacgcttactgttattaccagagtaccccgtaatttcaggtgaaatatccgcatacagagcaaaagcaatcgctgaagatgcaggcaacaacagccaggtaatcaacgctattgctaaacgctta

SEQ ID NO: 390 - protein, 462 aa, unidentified
  REGION 1..462: note = unclassified Partitiviridae
MGKSTHNISGYVFTDFQGELEDLHETHTERLQRESGVTYYDEFAKEELLDMYPNLYDQWIRDWSRSFYSGDKHLEAIYRYANQNLPITAVNQEVYADSIDAVRDGLRNLHIVRAHSVRTELDQVYYIQSSSAGYGYQGAKGPLSEYNHDRAIRRAKATLYSATAEDGEGIDHTIRESVPDVGYTRTQLTDLTEKTKVRSVWGRAFHYILLEGTSARPLLEGVMNSDTFIQIGSDPIVNVHCSNFDATVHRFEINTAFDQLKEHIYFPDFDTEMAFELSRQLFIHKKIAAPDGKIYWSHKGIPSGSYFTSLIGSIVNRLRIEYLWRLQCGRGPMTCKTLGDDSLIGDDIFYPPDKLQERALRLGWTINAEKTECSTNPASVTFLGRTIHGGQNQRDLKRCVRLLILPEYPVISGAISAFRAQAIAEDAGGNSQVINAIAKRLERRYGVASEEDVPSYFRRYQI

SEQ ID NO: 391 - DNA, 189 bp, Solanum tuberosum
  source 1..189: mol_type = genomic DNA
gtttgtttctgcttctacctttgatatatatataataattatcattaagtagtagtaatataatatttcaaatatttttttcaaaataaaagaatgtagtatatagcaattgcttttctgtagtttataagtgtgtatattttaatttataacttttctaatatatgaccaaaacatggtgatgtttag

(19) United States
(12) Patent Application Publication
(10) Pub. No.: US 2025/0230452 A1
(43) Pub. Date: Jul. 17, 2025

(54) POLYNUCLEOTIDES FOR MODIFYING ORGANISMS

(71) Applicant: FLAGSHIP PIONEERING INNOVATIONS VII, LLC, CAMBRIDGE, MA (US)

(72) Inventors: Arjun Devang Khakhar, Fort Collins, CO (US); Barry Andrew Martin, Newport, RI (US); Yajie Niu, Lexington, MA (US); Daniel Alexander Sprague, Cambridge, MA (US); Elizabeth Jane Antonelli Dennis, Watertown, MA (US); Mehmet Ali Halac, Philadelphia, PA (US); Yumeng Hao, Cambridge, MA (US); James Michael Kremer, Cambridge, MA (US); Jayashree Kumar, Somerville, MA (US); Chien-Yuan LIN, Cambridge, MA (US); Michka Gabrielle Sharpe, Cambridge, MA (US); Aditya Sushil Kumar Singh, Brookline, MA (US); Phu Tri Tran, Belmont, MA (US)

(21) Appl. No.: 18/730,112

(22) PCT Filed: Jan. 20, 2023
(86) PCT No.: PCT/US2023/060949

§ 371 Date: Jul. 18, 2024

Related U.S. Application Data
(60) Provisional application No. 63/266,967, filed on Jan. 20, 2022.
(60) Provisional application No. 63/379,056, filed on Oct. 11, 2022.

(51) Int. Cl. C12N 15/82; C12N 15/113

(52) U.S. Cl. CPC C12N 15/8203; C12N 15/113; C12N 2310/121

(57) ABSTRACT

Synthetic partitiviral satellite RNA molecules and satellite particles containing the same are disclosed. Also disclosed are methods of using the partitiviral satellite RNA molecules and satellite particles containing the same to change plant phenotypes, improve plant stress resistance, and improve plant pest and pathogen resistance.

### CROSS-REFERENCE TO RELATED APPLICATIONS

This international patent application claims benefit of U.S. provisional patent application No. 63/379,056, filed Oct. 11, 2022 and incorporated herein by reference in its entirety, and of U.S. provisional patent application No. 63/266,967, filed Jan. 20, 2022 and incorporated herein by reference in its entirety.

### INCORPORATION OF SEQUENCE LISTING

The instant application contains a Sequence Listing which has been submitted electronically in XML file format and is hereby incorporated by reference in its entirety. The XML file, created on Jan. 13, 2023, is named P13757WO00.xml and is 727,335 bytes in size. The sequence listing named P13757US01.xml which is 603,608 bytes in size for U.S. provisional patent application No. 63/379,056, filed Oct. 11, 2022, is incorporated herein by reference in its entirety. The sequence listing named VL70006_ST25 which is 441,582 bytes in size for U.S. provisional patent application No. 63/266,967, filed Jan. 20, 2022, is incorporated herein by reference in its entirety.

### BACKGROUND

There is need in the art for modifying polynucleotides for improving phenotypes and genotypes of organisms; in particular, for agricultural applications to improve plants such as crop plants.

Partitiviruses (PV) are dsRNA viruses having a bipartite genome which is separately encapsidated. One (PV) genome comprises a dsRNA encoding an RNA-dependent RNA polymerase and the other PV genome comprises a dsRNA encoding a capsid protein (Roossinck, M. doi/10.1111/nph.15364).

### SUMMARY

Recombinant DNA molecules comprising a first promoter which is operably linked to DNA encoding an RNA molecule, wherein the RNA molecule comprises from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP; wherein the 5′ RNA replication element, the cargo RNA molecule, and the 3′ RNA replication element are operably linked, wherein the promoter and the cargo RNA molecule are heterologous to the 5′ RNA replication element and the 3′ RNA replication element are provided. Cells, vectors, and agricultural formulations comprising the recombinant DNA molecules, vectors, or cells are also provided. Expression systems comprising the recombinant DNA molecules and a cell containing the recombinant DNA molecule and an RDRP protein that recognizes the 5′ and 3′ RNA replication elements encoded by the DNA molecule are also provided. Methods of providing a synthetic partitiviral satellite RNA to a plant, comprising contacting the plant with the recombinant DNA molecules, cells, vectors, or agricultural formulations thereof are also provided. Methods of producing an exogenous polypeptide in a plant or plant cell, comprising providing a plant or plant cell comprising the recombinant DNA molecule, wherein the cargo RNA molecule encoded by the DNA molecule comprises a translatable messenger RNA encoding the exogenous polypeptide, wherein the plant or plant cell comprises an RDRP protein that recognizes the 5′ RNA replication element and the 3′RNA replication element of the recombinant RNA and that catalyzes synthesis of a synthetic partitiviral satellite RNA from the recombinant RNA molecule, and wherein the exogenous polypeptide is translated from the translatable messenger are provided.

Recombinant RNA molecules comprising from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP, wherein the 5′ RNA replication element, the cargo RNA molecule, and the 3′ RNA replication element are operably linked and wherein the cargo RNA molecule is heterologous to the 5′ RNA replication element and the 3′ RNA replication element are also provided. Agricultural formulations and cells comprising the recombinant RNA molecules are also provided. Expression systems comprising: (a) an RNA molecule comprising the recombinant RNA molecule; and (b) a cell containing the recombinant RNA molecule and an RDRP protein that recognizes the 5′ and 3′ RNA replication elements of the recombinant RNA molecule are also provided. Plant propagules comprising the recombinant RNA molecules and a partitiviral RDRP are provided. Plant propagule comprising at least one plant cell comprising the recombinant RNA molecule and a partitiviral RDRP are provided. Methods of producing a modified plant propagule that comprises at least one plant cell comprising the recombinant RNA molecules, comprising isolating a plant propagule comprising at least one plant cell comprising the recombinant RNA molecule and a partitiviral RNA-dependent RNA polymerase (RDRP) from a mixed population of plant cells comprising both plant cells comprising the recombinant RNA molecule and plant cells lacking the recombinant RNA molecule are provided. Methods of providing a synthetic partitiviral satellite RNA to a plant comprising contacting the plant with the recombinant RNA molecule are also provided. Methods of obtaining a phenotypic change in a plant or plant cell comprising providing to a plant or plant cell the recombinant RNA molecule, wherein the cargo RNA molecule comprises RNA that effects a phenotypic change in the plant or plant cell in comparison to a plant or plant cell lacking the recombinant RNA, wherein the plant or plant cell comprises an RDRP protein that recognizes the 5′ RNA replication element and 3′ RNA replication element and catalyzes synthesis of a synthetic partitiviral RNA from the recombinant RNA molecule and the cargo RNA molecule effects the phenotypic change are provided. Methods of increasing a plant's resistance to a pest or pathogen comprising providing a plant with the recombinant RNA molecule, wherein the cargo RNA molecule effects an increase in the plant's resistance to a pest or pathogen in comparison to a plant lacking the recombinant RNA molecule, and wherein the plant or plant cell comprises an RDRP protein that recognizes the 5′ RNA replication element and the 3′ the 3′ RNA replication element of the recombinant RNA and catalyzes synthesis of a synthetic partitiviral satellite RNA from the recombinant RNA molecule are provided. Methods of increasing a plant's resistance to stress comprising providing a plant with the recombinant RNA molecule, wherein the plant comprises an RDRP protein that recognizes the 5′ RNA replication element and the 3′ the 3′ RNA replication element of the recombinant RNA and catalyzes synthesis of a synthetic partitiviral satellite RNA from the recombinant RNA molecule, and wherein the cargo RNA molecule effects an increase in the plant's resistance to stress relative to that in a plant lacking the recombinant RNA molecule are provided. Methods of manufacturing a synthetic partitiviral satellite particle, comprising: (a) providing to a plant cell at least one of the aforementioned recombinant RNA molecules, wherein the recombinant RNA molecule comprises an encapsidation recognition element (ERE), and wherein the plant cell comprises an RDRP protein that recognizes the 5′ RNA replication element and 3′ RNA replication element and catalyzes synthesis of a synthetic partitiviral satellite RNA from the recombinant RNA molecule and a viral capsid protein, wherein the ERE provides for encapsidation of the RNA by the viral capsid protein; and optionally (b) isolating the synthetic partitiviral satellite particle from the plant cell, a plant comprising the plant cell, or from media in which the plant cell or the plant had been grown are provided. Methods of manufacturing a synthetic partitiviral satellite particle, comprising combining an aforementioned recombinant RNA molecule with a viral capsid protein in a vessel, wherein the recombinant RNA molecule comprises an encapsidation recognition element (ERE), and wherein the ERE provides for encapsidation of the RNA by the viral capsid protein in the vessel; optionally wherein the method further comprises isolating the synthetic partitiviral satellite particle from uncombined RNA and/or viral capsid protein in the vessel are also provided. Methods of providing a synthetic partitiviral satellite RNA to a plant comprising: grafting a scion onto a rootstock comprising the recombinant RNA molecules, wherein at least one cell of the rootstock and/or the scion comprises the partitiviral RDRP, are provided. Methods for producing a plant that transmits a recombinant RNA molecule to progeny plants or seed comprising isolating an F₁ progeny plant or seed comprising at least one cell comprising a partitiviral RNA-dependent RNA polymerase (RDRP) and the recombinant RNA molecule from a population of F₁ plants or seed obtained from a parent plant comprising the recombinant RNA molecule are provided.

### BRIEF DESCRIPTION OF THE DRAWINGS

FIG. 1 shows a non-limiting embodiment of a structure of partitiviral satellite constructs. In this embodiment, the 5′ RNA replication element is labelled “5UTR Partitivirus” and the 3′ RNA replication element is labeled “3UTR Partitivirus.” The element labeled “tRNA” is a tRNA-like element, “Cargo” is a cargo RNA, and “MP” is a movement protein.

FIG. 2 shows a schematic of using Agrobacterium infiltration to generate a partitiviral satellite RNA, isolation of the encapsidated satellite RNA, and field application to plants. In this embodiment, the 5′ RNA replication element is labelled “5UTR Partitivirus” and the 3′ RNA replication element is labeled “3UTR Partitivirus.” The element labeled “tRNA” is a tRNA-like element, “Cargo” is a cargo RNA, and “MP” is a movement protein. The element labelled “Rep” refers to an RDRP coding region.

FIG. 3 shows the scheme used to establish a replicating synthetic partitivirus genome in Nicotiana benthamiana with Agrobacterium. “Rep” refers to an RDRP coding region.

FIG. 4 shows the analysis of partitivirus capsid protein RNA produced in Nicotiana benthamiana which received the DNA constructs of FIG. 3.

FIG. 5 shows the analysis of Partitivirus RdRP RNA produced in Nicotiana benthamiana which received the DNA constructs of FIG. 3.

FIG. 6 shows the scheme used to establish replication of a synthetic partitivirus genome comprising a cargo RNA molecule encoding a viral movement protein (MP) in Bloomsdale spinach containing a Partitivirus (SCV). In this embodiment, the 5′ RNA replication element is labelled “5UTR Partitivirus” and the 3′ RNA replication element is labeled “3UTR Partitivirus.”

FIG. 7 shows the analysis of cargo RNA in the 3rd systemic leaf at 56 days post-Agrobacterium infection in the SCV-infected Bloomsdale spinach. The arrow points to a band corresponding to an RT-PCR product of the correct size for the cargo RNA encoding the viral movement protein (MP).

FIG. 8 shows a T-DNA vector (SEQ ID NO: 255) comprising a cassette for expression of a PV satellite RNA encoding the PCV1 RDRP.

FIG. 9 shows a T-DNA vector (SEQ ID NO: 256) comprising a cassette for expression of a PV satellite RNA encoding the PCV1 CP.

FIG. 10 shows a T-DNA vector (SEQ ID NO: 257) comprising a cassette for expression of a PV satellite RNA encoding the SCV RDRP.

FIG. 11 shows a T-DNA vector (SEQ ID NO: 258) comprising a cassette for expression of a PV satellite RNA encoding the TMV MP.

FIG. 12 shows a T-DNA vector (SEQ ID NO: 259) comprising a cassette for expression of a PV satellite RNA encoding the SCV CP.

FIGS. 13A, B, C, and D show structural features identified in Pepper cryptic virus 1 (PCV1) 5′ and 3′ RNA replication elements (SEQ ID NOs: 372-375, respectively in FIGS. 13A, B, C, and D).

FIGS. 14A, B, C, and D show structural features identified in the Spinach cryptic virus 1 (SCV1) 5′ and 3′ RNA replication elements (SEQ ID NOs: 376-379, respectively in FIGS. 14A, B, C, and D).

FIG. 15A, B show structural features identified in the 5′ and 3′ RNA replication elements of a partitivirus from Zea mays Wu312 (SEQ ID NOs: 386 and 387, respectively in FIG. 15A, B).

### DETAILED DESCRIPTION

### Definitions

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “F₁,” “F₂,” and the like refer to plants or seed obtained from a parent plant which has been selfed or that has been crossed to another plant.

As used herein, the term “heterologous”, when used to describe a first element in reference to a second element means that the first element and second element do not exist in nature disposed as described. For example, a heterologous nucleic acid molecule or sequence is a nucleic acid molecule or sequence that (a) is not native to a cell in which it is expressed, (b) is linked or fused to a nucleic acid molecule or sequence with which it is not linked to or fused to in nature, or with which it is not linked to or fused to in nature in the same way, (c) has been altered or mutated by the hand of man relative to its native state, or (d) has altered expression as compared to its native expression levels under similar conditions. For example, a “heterologous promoter” is used to drive transcription of a sequence that is not one that is natively transcribed by that promoter (e.g., a eukaryote promoter used to drive transcription of a DNA molecule encoding a partitiviral RNA sequence); thus, a “heterologous promoter” sequence can be included in an expression construct by a recombinant nucleic acid technique. In other examples, a recombinant polynucleotide such as those provided by this disclosure can include genetic sequences of two or more different partitiviruses, which genetic sequences are “heterologous” in that they would not naturally occur together. In some embodiments “heterologous” refers to a molecule or to a discrete part of a molecule; for example, referring to a cargo RNA molecule (e.g., a nucleic acid such as a protein-encoding RNA, an ssRNA, a regulatory RNA, an interfering RNA, or a guide RNA), which can be part of a larger molecule, or referring to a structure (e.g., structures including a promoter (e.g., for a DNA dependent RNA polymerase or an RNA promoter dependent RNA polymerase), an RNA effecter, RNA cleavage agent recognition site, or a polynucleotide comprising or encoding an expression-enhancing element, encapsidation recognition element (ERE), selectable or scoreable marker, DNA aptamer, RNA aptamer; a transcription factor binding site, internal ribosome entry site (IRES), DNA spacer, an RNA cleavage agent recognition site, tRNA-like element, or a transcript-stabilizing or transcript-destabilizing RNA sequence) that is not found naturally in a plant partitivirus.

As used herein, the terms “include,” “includes,” and “including” are to be construed as at least having the features to which they refer while not excluding any additional unspecified features. The terms “comprise”, “comprises”, and “comprising” mean “include”, “includes”, and “including”, respectively.

As used herein, the term “internal ribosome entry site” or “IRES” refers to a sequence (e.g., an RNA sequence) capable of recruiting a ribosome and translation machinery to initiate translation from an RNA sequence. An IRES element is generally between 100-800 nucleotides. An appropriate IRES can be obtained from plant and plant viral IRES sequences such as encephalomyocarditis virus IRES (ECMV), maize hsp101 IRES 5′UTR, crucifer infecting tobamovirus crTMV CR-CP 148 IRES, tobacco etch virus (TEV) IRES 5′UTR and hibiscus chlorotic ringspot virus (HCRSV) IRES. In addition, in embodiments, an IRES sequence is derived from non-plant eukaryotic virus sequences that include but are not limited to: acute bee paralysis virus (ABPV), classical swine fever virus (CSFV), coxsackievirus B3 virus (CVB3), encephalomyocarditis virus (ECMV), enterovirus 71 (E71), hepatitis A virus (HAV), human rhinovirus (HRV2), human rhinovirus (HRV2), human lymphotropic virus (HTLV), polyoma virus (PV), and Zea mays (ZmHSP101). Examples of IRES sequence useful in the compositions and methods described herein are shown in Table 5.

As used herein, the phrase “operably linked” refers to a juxtaposition wherein the components so described are in a relationship permitting them to function in their intended manner. For instance, a promoter is operably linked to a coding sequence if the promoter provides for transcription or expression of the coding sequence.

As used herein the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32 (5): 1792-1797, 2004). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul et al. (1990) J. Mol. Biol., 215:403-410). As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows: 100 multiplied by (the fraction X/Y), where X is the number of nucleotides or amino acids scored as identical matches by a sequence alignment program (e.g., BLAST) in that program's alignment of A and B, and where Y is the total number of nucleotides or amino acids in B.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

As used herein, the term “transcriptome” refers to the sum total of all RNA molecules expressed in a cell. Such RNA molecules include mRNAs, tRNAs, ribosomal RNAs, miRNAs, viral RNAs (both genomic and sub-genomic), and long non-coding RNAs.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Unless otherwise stated, nucleic acid sequences described herein are given, when read from left to right, in the 5′ to 3′ direction. Nucleic acid sequences may be provided as DNA or as RNA, as specified. Furthermore, because of known codon degeneracy, different nucleic acid sequences can encode the same polypeptide sequence, and such modified nucleic acid sequences (e.g., for the purposes of codon optimization for a given species) are within the scope of the present disclosure. Where a term is provided in the singular, it also contemplates aspects of the invention described by the plural of that term.

This disclosure provides, inter alia, recombinant polynucleotides (e.g., recombinant DNA, recombinant RNA, recombinant ssRNAs, recombinant dsRNAs, recombinant vectors, etc.) including one or more sequences of or derived from a partitivirus (PV); in particular, a 5′ or 3′ RNA replication element that is recognized by a partitiviral RNA-dependent RNA polymerase (RDRP). This disclosure is further related to methods of making and using such recombinant polynucleotides, for example, by employing such recombinant polynucleotides to express a heterologous cargo sequence in a plant and optionally thereby modifying expression of an endogenous target sequence and/or genotype or phenotype of the plant. In embodiments, the partitivirus is a commensal partitivirus, that is, a partitivirus that is endemic or native to a given eukaryote host (such as a host plant) without causing apparent negative effects on the host (i.e., is considered non-pathogenic), is often present at a persistent but low population (i.e., low viral titer), and is often vertically transmitted to succeeding generations of the host.

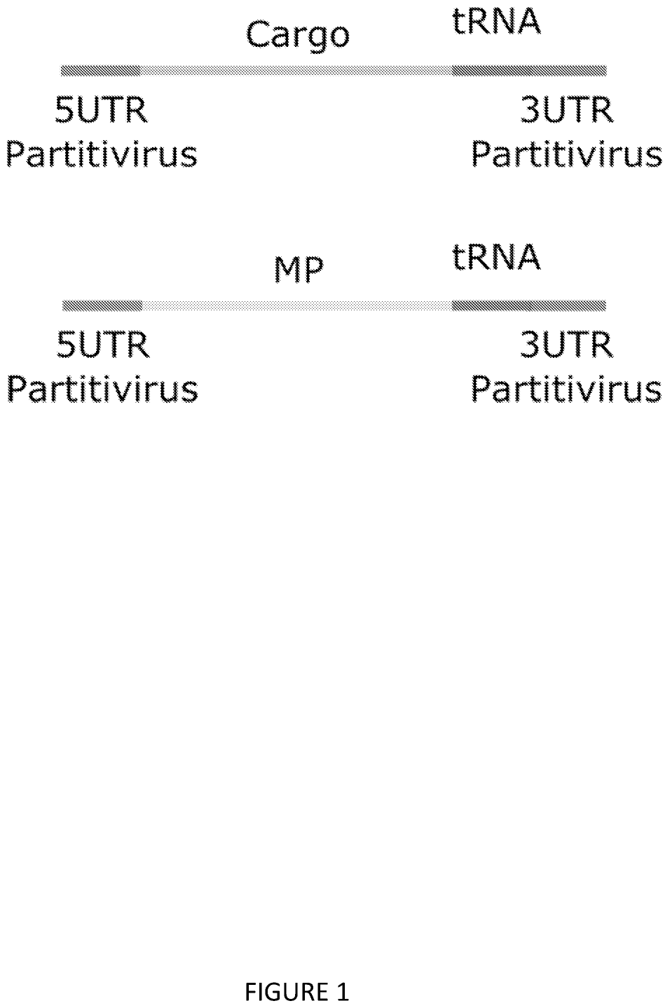

In one aspect, this disclosure is related to a recombinant DNA molecule that includes a promoter that is functional in a cell and is operably linked to a DNA sequence encoding an RNA molecule. The RNA molecule includes, in 5′ to 3′ order: (a) a 5′ RNA replication element that is capable of being recognized by a partitiviral RNA-dependent RNA polymerase (RDRP); (b) a cargo RNA sequence; and (c) a 3′ RNA replication element that is capable of being recognized by the partitiviral RDRP. FIG. 1 shows an embodiment of a generalized structure of a DNA polynucleotide encoding a partitiviral satellite, where the 5′ RNA replication element corresponds to the 5′ untranslated region (UTR) of a partitivirus and where the 3′ RNA replication element corresponds to the 3′ untranslated region (UTR) of a partitivirus.

Recombinant DNA molecules provided herein can include a promoter that is functional in a cell (e.g., a bacterial cell, a plant cell, a fungal cell, or an animal cell) and is operably linked to a DNA sequence encoding an RNA molecule (e.g. a 5′ RNA replication element, a cargo RNA sequence; and a 3′ RNA replication element; a ribozyme, an intron, or a RNA encoding a protein (e.g., a capsid, movement, RDRP, or RPDRP protein).

In embodiments, a promoter functional in a plant cell can provide for cell-, tissue-, or organ-specific gene expression or expression that is inducible by external signals or agents (for example, light-, pathogen-, wound-, stress-, or hormone-inducible elements, or chemical inducers), or elements that are capable of cell-cycle regulated gene transcription; such elements may be located in the 5′ or 3′ regions of the native gene or engineered into a polynucleotide.

Promoters include those from viruses, bacteria, fungi, animals, and plants. Suitable promoters can be used to drive expression by any RNA polymerase (e.g., RNA pol I, pol II, or pol III) Embodiments of promoters include those from cauliflower mosaic virus (e.g., p35S), bacteriophage (e.g., pT7), and plants (e.g., pATUBQ10). In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see, e.g., Ferré-D'Amare and Scott (2010) Cold Spring Harbor Perspectives Biol., 2: a003574). In certain embodiments, the promoter is a pol II promoter operably linked to a nucleotide sequence encoding the RNA. In certain embodiments, the promoter operably linked to one or more polynucleotides encoding elements of a genome-editing system is a constitutive promoter that drives DNA expression in plant cells. In certain embodiments, the promoter drives DNA expression in the nucleus or in an organelle such as a chloroplast or mitochondrion. Examples of constitutive promoters active in plant cells include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and a nopaline synthase (NOS) and octopine synthase (OCS) promoter from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PDK) promoter, which is active in the chloroplasts of mesophyll cells. In some embodiments, the promoter is heterologous to the cell it is functional in and/or to the other elements to which the promoter is operably linked. All of the patent publications referenced in this paragraph are incorporated herein by reference in their entirety.

Recombinant polynucleotides provided herein can comprise or encode RNA molecules containing 5′ and 3′ RNA replication elements recognized by a partitiviral RNA-dependent RNA polymerase (RDRP). In certain embodiments, recognition by a partitiviral RDRP can be identified in an in vitro RDRP assay (e.g., an assay adapted from Horiuchi et al. Plant Cell Physiol. 42 (2): 197-203, 2001). In certain embodiments, recognition by a partitiviral RDRP can be identified by an in vivo RDRP assay wherein an RNA comprising 5′ and 3′ RNA replication elements is introduced into a cell comprising the RDRP and replication of the RNA is assayed (e.g., by an RT-PCR assay or an assay for a reporter gene encoded by a cargo RNA located in the RNA comprising 5′ and 3′ RNA replication elements). In certain embodiments, cells comprising the RDRP can be engineered by introducing a gene or RNA molecule encoding the RDRP into the cell. In other embodiments, the cell comprising the RDRP can be a cell which contains a partitivirus which expresses the RDRP. In certain embodiments, the recombinant polynucleotides (e.g., recombinant DNAs or recombinant RNAs) comprise a 5′ RNA replication element and a 3′ RNA replication element obtained from the same partitiviral genome or from related partitiviral genomes having at least 85%, 90%, 95%, 98%, or 99% sequence identity to one another. In certain embodiments, the recombinant polynucleotides (e.g., recombinant DNAs or recombinant RNAs) comprise a 5′ RNA replication element, a 3′ RNA replication element, and an RDRP are obtained from the same partitiviral genome or from partitiviral genomes having at least 85%, 90%, 95%, 98%, or 99% sequence identity to one another. Non-limiting examples of a 5′ RNA replication element and a 3′ RNA replication element from the same partitiviral capsid protein genome include those set forth in each row of Table 9. Non-limiting examples of a 5′ RNA replication element and a 3′ RNA replication element from the same partitiviral RDRP genome and the corresponding RDRP protein that can recognize that 5′ RNA replication element and 3′ RNA replication element include those set forth in each row of Table 10. The RDRP set forth in Table 10 can also recognize the corresponding 5′ RNA replication element and a 3′ RNA replication element from the partitiviral capsid protein genome corresponding to the same partitivirus (i.e., a Partitivirus having the capsid protein genome of Table 9 and the RDRP genome of Table 10). In certain embodiments, the recombinant polynucleotides (e.g., recombinant DNAs or recombinant RNAs) comprise a 5′ RNA replication element, a 3′ RNA replication element, and/or an RDRP coding region are obtained from two partitiviral genomes wherein the members of each pair of the 5′ RNA replication elements, 3′ RNA replication elements, and RDRP coding regions of the two partitiviral genomes have at least 85%, 90%, 95%, 98%, or 99% sequence identity to one another. In certain embodiments, the recombinant polynucleotides (e.g., recombinant DNAs or recombinant RNAs) comprise a 5′ RNA replication element and a 3′ RNA replication element obtained from distinct partitiviral genomes. In certain embodiments, the recombinant polynucleotides (e.g., recombinant DNAs or recombinant RNAs) comprise a 5′ RNA replication element, a 3′ RNA replication element, and an RDRP are obtained from distinct partitiviral genomes. In certain embodiments, the distinct partitiviral genomes will have less than 85%, 80%, 75%, or 70% sequence identity to one another. In certain embodiments, the distinct partitiviral genomes will have 50%, 60%, or 65% to any one of 70%, 75%, 80%, or 84% sequence identity to one another.

In certain embodiments, the combination of 5′ RNA replication elements, 3′ RNA replication elements, and RDRP set forth in a single row of Table 10, variants thereof having at least 85%, 90%, 95%, 98%, or 99% sequence identity to the 5′ RNA replication element, 3′ RNA replication elements, and RDRP, or variants thereof wherein secondary structures of the RNA replication elements are conserved are used together in an expression system, plant cell, plant propagule, plant, or method provided herein. In certain embodiments, the 5′ RNA replication elements and 3′ RNA replication elements in a given row or variants thereof are operably linked to a cargo RNA and replicated by the corresponding RDRP or variant thereof in the row. In certain embodiments, the combination of 5′ RNA replication elements, 3′ RNA replication elements, and RDRP set forth in any one of rows 1 to 20 or aforementioned or otherwise disclosed variants thereof are used in a dicot plant cell-based expression system, dicot plant cell, dicot plant propagule, dicot plant, or related dicot plant-based method provided herein. In certain embodiments, the aforementioned dicot is a member of the genus Brassica, Capsicum, Cucumis, Cucurbita, Hordeum, Gossypium, Nicotiana, Solanum, or Glycine. In certain embodiments, the combination of 5′ RNA replication elements, 3′ RNA replication elements, and RDRP set forth in row 21 (i.e., SEQ ID NO: 386, 387, and 390, respectively) or aforementioned or otherwise disclosed variants thereof are used in a monocot plant cell-based expression system, monocot plant cell, monocot plant propagule, monocot plant, or related monocot plant-based method provided herein. In certain embodiments, the aforementioned monocot is a member of the genus Avena, Hordeum, Oryza, Secale, Triticum, Sorghum, or Zea.

Examples of DNA molecules which encode RNA molecules comprising or containing 5′ and 3′ RNA replication elements recognized by a partitiviral RDRP are set forth in Table 1. DNA molecules which encode RNAs comprising or containing 5′ RNA replication elements recognized by a partitiviral RDRP include SEQ ID NO: 1 (PV Capsid Protein genome 5′UTR consensus), SEQ ID NOs: 2-34, 374, 378, and 380 (PV Capsid Protein genome 5′ UTRs), SEQ ID NOs: 69 (PV RdRP genome 5′ UTR consensus), and SEQ ID NOs: 70-88, 372, 376, 382, and 386 (PV RdRP genome 5′ UTRs). DNA molecules which encode RNAs comprising or containing 3′ RNA replication elements recognized by a partitiviral RDRP include SEQ ID NO: 35 (PV Capsid Protein genome 3′ UTR consensus), SEQ ID NOs: 36-68, 375, 379, and 381 (PV Capsid protein genome 3′ UTRs), SEQ ID NOs: 89 (PV RdRP genome 3′ UTR consensus), and SEQ ID NOs: 90-108, 373, 377, 383, and 387 (PV RDRP genome 3′ UTRs).

Structural features (e.g., dsRNA hairpins and ssRNA loops) identified in partitiviral 5′ and 3′ RNA replication elements are shown in Table 1 by way of dot bracket notation. The dot bracket notation provided in Table 1 was generated using RNA Fold software for predicting RNA secondary structure based on minimum free energy predictions of base pair probabilities. A dot ‘.’ signifies an unpaired base and a bracket ‘(’ or ‘)’ represents a paired base. Dot bracket notation is further described in Mattei et al., Nucleic Acids Research, 42 (10): 6146-6157, 2014; Ramlan and Zauner In International Workshop on Computing With Biomolecules, E. Csuhaj-Varju, R. Freund, M. Oswald and K. Salomaa (Eds.), 27 Aug. 2008, Wien, Austria, pp. 75-86, From: Austrian Computer Society, 2008; and Hofacker et al., Monatshefte Fur Chemie Chem. Monthly, 125:167-188, 1994. Such structural features can range in size from 20, 30, or 40 to about 500 nucleotides (nt). These structural features are useful for designing engineered polynucleotide sequences that function as partitiviral RNA replication elements and/or for constructing variants of the sequences set forth in SEQ ID NO: 1 to 108, 372 to 382, 386, and 387 that function as 5′ and 3′ RNA replication elements. In certain embodiments, one of more residues in the RNA secondary structure set forth in Table 1 or in equivalent RNAs are substituted with distinct nucleotides which maintain the RNA secondary structure (e.g., presence or absence of base pairing). In certain embodiments the RNA secondary structure set forth in Table 1 or in equivalent RNAs, the RNA secondary structure is maintained by making substitutions in the nucleotide sequence that result in no changes in the position of base-paired nucleotides or non-base-paired nucleotides. In certain embodiments the RNA secondary structure set forth in Table 1 or in equivalent RNAs is maintained by substituting nucleotides in the secondary structure which are not base paired with nucleotides which will not base pair and/or by substituting nucleotides in the secondary structure which are base paired with nucleotides which will base pair. In this context, it is understood that maintaining the RNA secondary structure need not be absolute (e.g., the structure can be partially maintained). In certain embodiments, a dsRNA structure is partially maintained when one, two, three or more nucleotides, particularly at the 5′ end and/or 3′ end of a hairpin-forming structure are substituted with nucleotides which do not base pair and thus reduce the total length of dsRNA in the structure. In certain embodiments, an unpaired RNA structure is partially maintained when one, two, three or more nucleotides, particularly at the 5′ end and/or 3′ end of a loop structure are substituted with nucleotides which base pair and thus reduce the total length of ssRNA in the loop structure. Embodiments of partitiviral satellite RNAs include those where the 5′ RNA replication element includes one or more of these 5′ structural features and/or wherein the 3′ RNA replication element includes one or more of these 3′ structural features. In certain embodiments, the 5′ RNA replication elements comprise an RNA encoded by a DNA having at least 70%, 75%, 80%, 85%, 90%, 95%, 98%, 99%, or 100% sequence identity to SEQ ID NO: 1-34, 69-88, 372, 374, 376, 378, 380, 382, or 386, optionally wherein the encoded RNA maintains or partially maintains a corresponding structural feature set forth in Table 1. In certain embodiments, the 3′ RNA replication elements comprise an RNA encoded by a DNA having at least 70%, 75%, 80%, 85%, 90%, 95%, 98%, 99%, or 100% sequence identity to SEQ ID NO: 35-68, 89-108, 373, 375, 377, 379, 381, 383, or 387, wherein the encoded RNA optionally maintains or partially maintains a corresponding structural feature set forth in Table 1.

Recombinant polynucleotides (e.g., recombinant DNA, recombinant RNA, recombinant ssRNAs, recombinant dsRNAs, recombinant vectors, etc.) provided herein can also comprise or encode additional RNA elements.

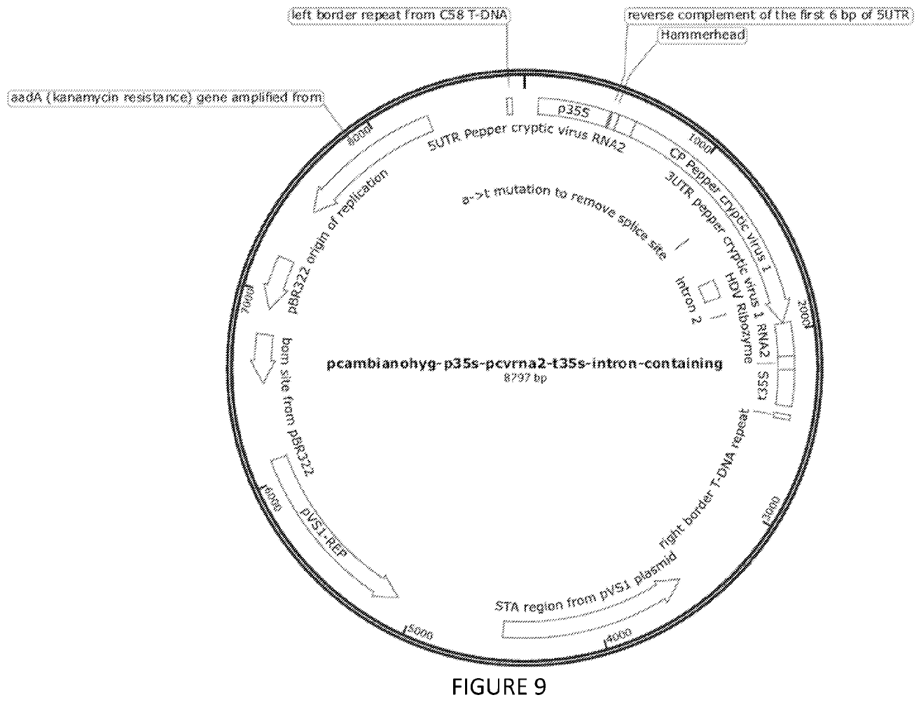

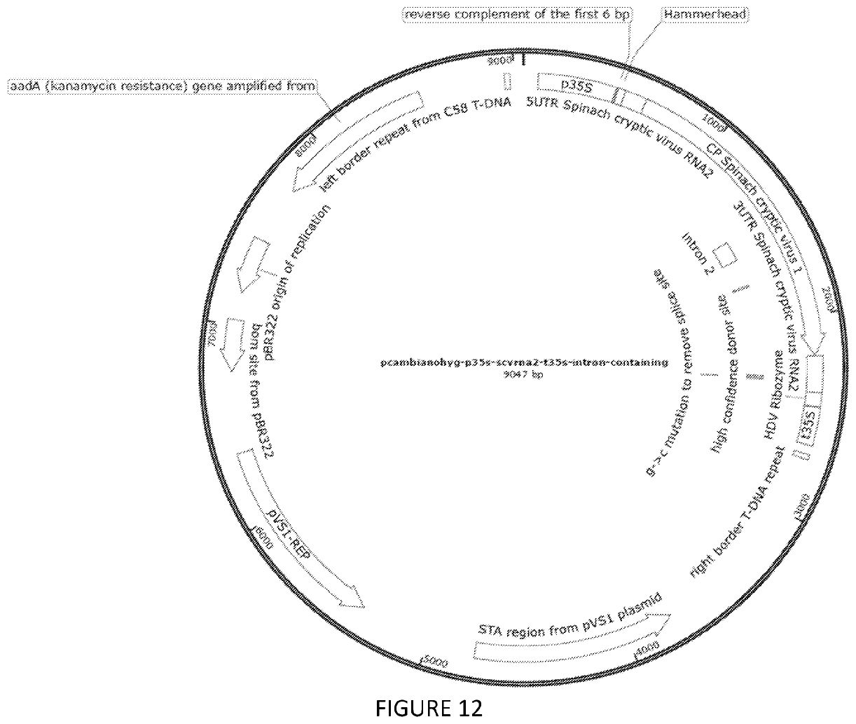

Such aforementioned additional RNA elements can include RNAs encoding a partitiviral CP. Examples of DNA sequences encoding partitiviral CP include the corresponding sequences of PV CP genomes set forth in Table 1 under descriptors “NC_XXXXXX” which refer to the National Center for Biotechnology Information database accession number for entries in the world wide web internet database “ncbi.nlm.nih.gov/nuccore.” Examples of DNA sequences encoding partitiviral CP also include the sequence of the PCV1 CP disclosed in FIG. 9 and SEQ ID NO: 256 as well as DNA sequences having at least 85%, 90%, 95%, 98%, or 99% identity thereto. Examples of DNA sequences encoding partitiviral CP also include the sequence of the DNA encoding the SCV CP disclosed in FIG. 12 and SEQ ID NO: 259 as well as DNA sequences having at least 85%, 90%, 95%, 98%, or 99% identity thereto. Examples of DNA sequences encoding partitiviral CP and partitiviral CP sequences also include the sequences set forth in Table 7 as well as the DNA and protein sequences having at least 85%, 90%, 95%, 98%, or 99% identity thereto.

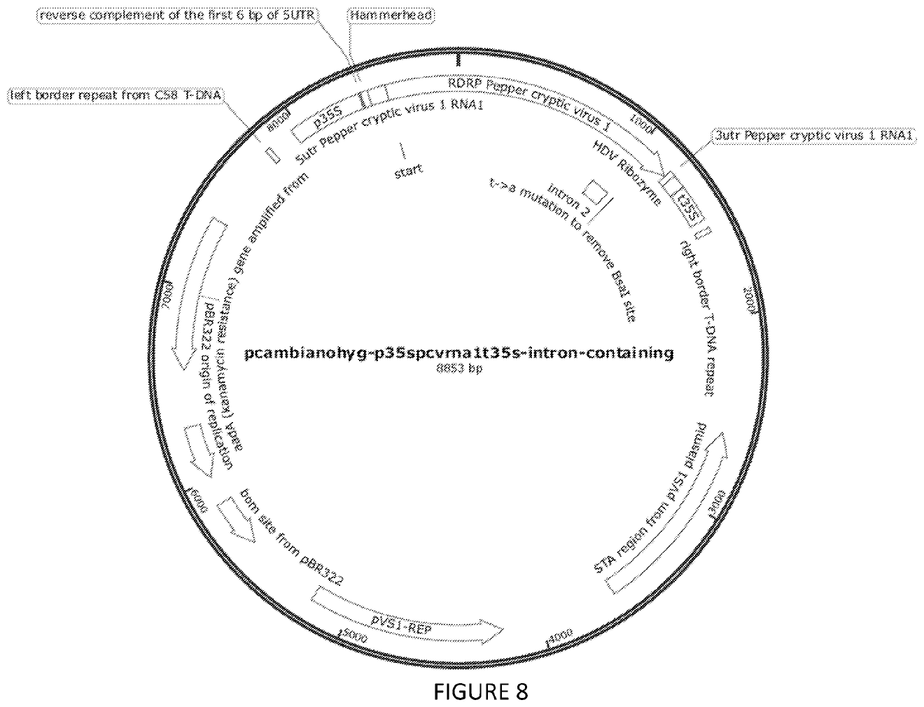

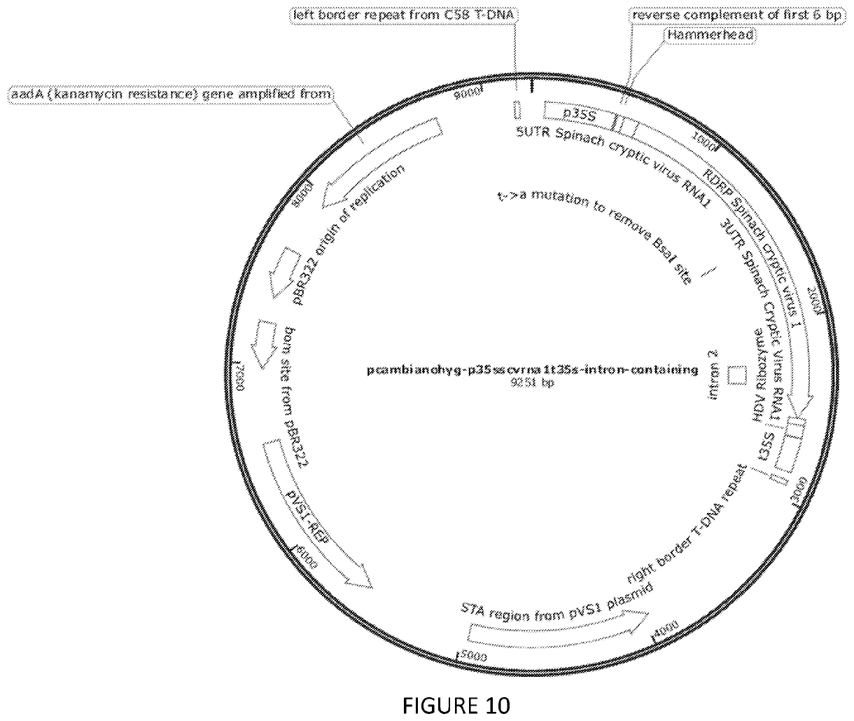

Such aforementioned additional RNA elements can include RNAs encoding a partitiviral RDRP. Examples of DNA sequences encoding partitiviral include the corresponding sequences of PV RDRP genomes set forth in Table 1 under descriptors “NC_XXXXXX” which refer to the National Center for Biotechnology Information database accession number for entries in the world wide web internet database “ncbi.nlm.nih.gov/nuccore.” Examples of DNA sequences encoding partitiviral RDRP also include the sequence of the DNA encoding PCV1 RDRP disclosed in FIG. 8 and SEQ ID NO: 255 as well as DNA sequences having at least 85%, 90%, 95%, 98%, or 99% identity thereto. Examples of DNA sequences encoding partitiviral RDRP also include the sequence of the DNA encoding the SCV RDRP disclosed in FIG. 10 and SEQ ID NO: 257 as well as DNA sequences having at least 85%, 90%, 95%, 98%, or 99% identity thereto. Examples of DNA sequences encoding partitiviral RDRP and partitiviral RDRP protein sequences also include the sequences set forth in Table 8 as well as the DNA and protein sequences having at least 85%, 90%, 95%, 98%, or 99% identity thereto.

Such aforementioned additional RNA elements can include RNAs encoding a viral movement protein (MP). In certain embodiments, the cargo RNA comprises an RNA encoding a viral MP. Without being bound to hypothesis or theory, the viral movement protein is believed to bind to the RNA and to assist its movement (and thus the movement of the cargo RNA) throughout the plant, e.g., via the plasmodesmata. MPs include tobacco mosaic virus (TMV), cowpea mosaic virus, potato leafroll virus, tomato spotted wilt virus, and tomato mosaic virus MPs. MPs from a variety of viruses are described in Table 3.

Such aforementioned additional RNA elements can include tRNA-like sequences (TLS). TLS can trigger mobility of otherwise nonmobile RNAs, assisting to increase systemic delivery of the RNA molecule. TLS include tRNAs and tRNA-like sequences identified from other genetic elements, e.g., mRNAs. An isoleucine tRNA encoded by SEQ ID NO: 249 is an example of a useful tRNA-like sequence. Other mobile RNAs including TLS identified in Arabidopsis which are useful for building polynucleotides are described in Table 4. In assembling Table 4, mobile mRNA sequences were downloaded from the PLAMOM database for Arabidopsis. The tRNA “seed alignment” from the RFAM database was downloaded in stockholm format (multiple sequence alignment+secondary structure). A covariance model was created with INFERNAL for the tRNA stockholm alignment. PLAMOM mRNA sequences were scanned for significant similarity to tRNAs based on primary and secondary structure features. mRNA sequences with significant hits (E-val<1) were then saved to a fasta file. In one embodiment, such a tRNA-like sequence includes a tRNA-like sequence from an Arabidopsis Flowering Time T (FT) mRNA. In some embodiments, the RNA molecule includes at least one RNA encoding a viral MP, a tRNA-like sequence from an Arabidopsis FT mRNA, and an encapsidation recognition element (ERE) comprising TMV-OAS. In some embodiments, the RNA molecule includes a tRNA-like sequence selected from the group consisting of SEQ ID NOs: 184-231. In some embodiments, the RNA molecule includes a modified tRNA-like sequence that has at least 90%, 95%, 98%, or 99% sequence identity to a scaffold tRNA-like sequence selected from the group consisting of SEQ ID NOs: 184-231 and that maintains the secondary structure of the scaffold tRNA-like sequence.

Such aforementioned additional RNA elements can include RNAs encoding a viral capsid protein (CP). Such capsid proteins are also sometimes referred to as coat proteins, with both capsid and coat proteins being referred to as “CP.” In certain embodiments, the cargo RNA comprises an RNA encoding a viral CP. CP can be provided, e.g., by co-expression of a recombinant construct encoding the CP or by native expression by a virus endogenous to or introduced into a plant cell. Encapsidation of an RNA molecule by the CP is achieved provided it contains an encapsidation recognition element (ERE), e.g., an origin of assembly sequence (OAS). Table 2 describes several OAS and CP sequences from a variety of viruses useful in engineering constructs which provide for RNA encapsidation. In embodiments, the OAS is positioned near the 3′ end of a construct, e.g., within the 3′ region of a cargo RNA or 3′ to a cargo RNA. For example, in some embodiments, the OAS is found 5′ to the 3′ RNA replication elements (e.g., the 3′ RNA replication elements set forth in Table 1). In embodiments, a TMV-OAS positioned at the 3′ end of the RNA molecule is recognized by the TMV capsid protein, leading to assembly of a TMV virion around the RNA.

Embodiments wherein the recombinant RNAs are complexed with RNA binding proteins (RBP) are also provided herein. Such RBP can comprise RNA recognition motifs (RRM) such as: (i) Lys/Arg-Gly-Phe/Tyr-Gly/Ala-Phe/Tyr-Val/Ile/Leu-X-Phe/Tyr, where X can be any amino acid (SEQ ID NO: 253); (ii) Ile/Val/Leu-Phe/Tyr-Ile/Val/Leu-X-Asn-Leu, where X can be any amino acid (SEQ ID NO: 254). Such RBP and RRM include those disclosed in Maris et al. 2005, doi.org/10.1111/j. 1742-4658.2005.04653.x.

Such aforementioned additional RNA elements can include at least one ribozyme. Ribozymes include self-cleaving ribozyme, a ligand-responsive ribozyme (aptazyme), a trans-cleaving ribozyme designed to cleave a target sequence (e.g., a trans-cleaving hammerhead ribozyme designed to cleave the pepper phytoene desaturase (PDS) sequence (the RNA encoded by SEQ ID NO: 250), a hepatitis delta virus (HDV) ribozyme (the RNA encoded by SEQ ID NO: 251), or a hammerhead ribozyme (the RNA encoded by SEQ ID NO: 252). In various embodiments, multiple ribozymes are included in a polynucleotide. In certain embodiments, such a ribozyme (e.g., a self-cleaving ribozyme) is located 5′ to the 5′ RNA replication element and/or 3′ to the 3′ RNA replication element in the recombinant RNA. FIG. 10 and the associated SEQ ID NO: 257 depict use of ribozymes in a vector encoding a recombinant RNA.

Such aforementioned additional RNA elements can include intronic sequences are included in an engineered construct. Examples of intronic sequences are described in Table 6. In certain embodiments, intronic sequences are placed in a 5′UTR downstream of a promoter (e.g., a promoter active in plant cells) used to drive expression of a recombinant RNA. In certain embodiments, intronic sequences are placed 5′ to a 5′ RNA replication element, in a cargo RNA, or 3′ to a 3′ RNA replication element.

Such aforementioned additional RNA elements can include RNA promoters recognized by an RNA Promoter Dependent RNA polymerase (RPDRP) and/or RNA molecules encoding an RNA Promoter Dependent RNA polymerase (RPDRP). Examples of such RNA promoters and RPDRP include a Brome Mosaic Virus RNA promoter and RPDRP (Siegal et al. 1998, doi: 10.1073/pnas.95.20.11613). In certain embodiments, such RNA promoters can be placed either 5′ and/or 3′ to an RNA molecule comprising a 5′ replication element, a cargo RNA, and a 3′ replication element to permit production of either or both + and − strands of the RNA molecule when the RPDRP is provided. In certain embodiments, such RNA promoters can be operably linked to a cargo RNA molecule and/or to any additional RNA element to permit production of the corresponding cargo and/or additional RNA when the RPDRP is provided.

Other elements in the recombinant polynucleotides provided herein include: a) a discrete expression cassette including a second promoter operably linked to a DNA sequence to be transcribed, and optionally a terminator element (see, e.g., a NOS or CaMV35S terminator); (b) an expression-enhancing element (e.g., a DNA encoding an expression-enhancing intronic sequence); (c) a DNA or RNA sequence encoding a marker (e.g., a selectable marker such as DNA or RNA encoding an antibiotic resistance or herbicide resistance sequence; DNA encoding a scorable marker or detectable label (e.g., a beta-glucuronidase, fluorescent protein, luciferase, etc.); (d) a DNA aptamer; (e) a DNA or RNA sequence encoding an RNA aptamer; (f) T-DNA left and right border DNA sequences; (g) a spacer DNA sequence; (h) a DNA sequence encoding a transcription factor binding site; (i) a DNA sequence encoding a localization sequence (e.g., DNA encoding a targeting peptide, such as a nuclear localization signal (NLS), a mitochondrial localization signal, or a plastid localization signal); or (j) a DNA sequence encoding at least one sequence-specific recombinase recognition site (SSRRS: e.g., a pair of sequence-specific recombinase recognition sites that are recognized by a given recombinase, such as LOX sites recognized by a CRE recombinase); and (k) a DNA sequence encoding a transcript-stabilizing or transcript-destabilizing sequence (see, e.g., US Published Patent Application 2007/0011761, incorporate by reference in its entirety; Geisberg et al. Cell (2014) 156:812-824).

Provided herein are recombinant polynucleotides comprising a cargo RNA molecule or comprising DNA encoding a cargo RNA molecule. In some embodiments, the recombinant polynucleotide includes a single cargo RNA molecule. In other embodiments, the recombinant polynucleotide includes at least two cargo RNA molecules, e.g., 2, 3, 4, 5, 6, 7, 8, 9, 10, or more than 10 cargo RNA molecules.

In certain embodiments, a cargo RNA molecule is up to about 3.2 kilobases (kb) in length. Cargo RNA molecules can range in length from any one of about 20 nucleotides (nt), 100 nt, 200 nt, 300 nt, 400 nt, 500 nt, 600 nt, 700 nt, 800 nt, or 900 nt to any one of about 1 kb, 2 kb, 3 kb, or 3.2 kb in length. Other lengths of the cargo RNA molecule are less than or equal to 100 nucleotides (nt) can range in length from any one of about 20 nt, 30 nt, or 40 nt to any one of about 50 nt, 60 nt, 70 nt, 80 nt, 90 nt, or 100 nt in length. Recombinant RNAs comprising a cargo RNA of up to about 3.2 kb in length can in certain embodiments be encapsidated by a PV capsid protein. In such embodiments, cargo RNAs can range in length from any one of about 20 nt, 100 nt, 200 nt, 300 nt, 400 nt, 500 nt, 600 nt, 700 nt, 800 nt, or 900 nt to any one of about 1 kb, 2 kb, 3 kb, 3.2 kb, 4 kb, 5 kb, 6 kb, 7 kb, 8 kb, 9 kb, 10 kb, 11 kb, 12 kb, 13 kb, or 14 kb in length. Recombinant RNAs comprising a cargo RNA of up to about 3.2 kb, 4 kb, 5 kb, 6 kb, 7 kb, 8 kb, 9 kb, 10 kb, 11 kb, 12 kb, 13 kb, or 14 kb in length can in certain embodiments be encapsidated by a heterologous viral capsid protein set forth in Table 3. In certain embodiments, recombinant RNAs comprising a cargo RNA of up to about 14 kb and encapsidated by a heterologous viral capsid protein can comprise an OAS element set forth in Table 2 and be encapsidated by a corresponding capsid protein set forth in Table 2. In some embodiments, the cargo RNA molecule is greater than 14 kb, for example, 15 kb, 16 kb, 17 kb, 18 kb, 19 kb, or even 20 kb. In embodiments, the cargo RNA molecule includes: (a) at least one coding sequence, (b) at least one non-coding sequence, or (c) both at least one coding sequence and at least one non-coding sequence. Such cargo RNA molecules include combinations of coding/non-coding sequence; multiple non-coding/coding sequences; as well as aptamers, ribozymes, and other elements as is described herein.

In embodiments, the cargo RNA molecule includes at least one coding sequence (e.g., a translatable sequence). In some embodiments, the coding sequence is accordingly a protein or a polypeptide. In some embodiments, a cargo RNA comprises a selectable marker RNA encoding an antibiotic resistance or herbicide resistance polypeptide sequence or a scorable marker RNA encoding a scorable marker protein (e.g., a beta-glucuronidase, fluorescent protein, luciferase, etc.). Examples of selectable marker/selection agent combinations include glyphosate-resistant EPSPS enzymes and/or glyphosate oxidases/glyphosate, a bialaphos resistance (bar) or phosphinothricin acyl transferase (pat) enzyme/glufosinate, or a neomycin phosphotransferase (npt)/neomycin or kanamycin. Examples of scorable markers include β-glucuronidase (GUS), luciferase, and fluorescent proteins such as green fluorescent protein (GFP), yellow fluorescent protein (YFP), and cyan fluorescent protein (CFP). In certain embodiments, the cargo RNA includes a selectable or scorable RNA marker, such as an RNA aptamer or a regulatory RNA, such as an siRNA or siRNA precursor (see, e.g., U.S. Pat. Nos. 8,404,927, 8,455,716, 9,777,288, 10,378,012), a miRNA or a miRNA precursor (see, e.g., U.S. Pat. Nos. 8,410,334, 8,395,023, 9,708,620), a trans-acting siRNA or trans-acting siRNA precursor (see, e.g., U.S. Pat. Nos. 8,030,473, 8,476,422, 8,816,061, 9,018,002), a phased sRNA or phased sRNA precursor (see, e.g., U.S. Pat. No. 8,404,928), an siRNA or miRNA decoy (see, e.g., U.S. Pat. Nos. 8,946,511, 9,873,888), an siRNA or miRNA cleavage blocker (see, e.g., U.S. Pat. No. 9,040,774), an siRNA or miRNA recognition and cleavage sequence (see, e.g., U.S. Pat. Nos. 8,334,430, 9,139,838, 9,976,152, 10,793,869, 10,876,126), a riboswitch, or a ribozyme. Suitable RNA aptamers include those that exhibit fluorescence upon binding a molecule. For example, the fluorescent RNA aptamer can be the Broccoli RNA aptamer. Other fluorescent RNA aptamers that can be used include, but are not limited to, Spinach, Spinach2, Carrot, Radish, Corn, Red Broccoli, Orange Broccoli, and Broccoli Fluorets. Suitable regulatory RNAs can be used to down-regulate (i.e., silence) the expression of a marker gene. For example, phytoene desaturase (PDS) is widely used as a marker gene because silencing of the gene yields a photobleached phenotype. Regulatory RNAs such as decoys or cleavage blockers can also be used to interfere with endogenous small RNA-regulated pathways, resulting in a visible phenotype; see, e.g., U.S. Pat. Nos. 8,946,511, 9,873,888, 9,040,774). In embodiments, the cargo RNA sequence encodes at least one protein or polypeptide that provides a desirable trait in a plant in which the protein or polypeptide is expressed. Non-limiting examples of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of beneficial insects (such as honeybees and silkworms) or for decreasing the fitness of pest invertebrates (such as aphids, caterpillars, beetle larvae, and mites). Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides and/or nematicidal polypeptides) for controlling invertebrate pests such as insects or nematodes. Embodiments of antimicrobial polypeptides include cathelicidins, cecropins, beta-defensins, amphibian antimicrobial peptides (e.g., aurein-like peptides, esculentin, gaegurin, brevinin, rugosin, ranatuerin, ranacyclin, uperin, ocellatin, grahamin, nigrocin, dermoseptin, temporin, bombinin, maximin), enterocins, ponicerins, megourins, apidaecins, abaecins, attacin, bacteriocins and lantibiotics, dermcidin, formaecin, halocidins, lactocin, tachystatins, and some insecticidal toxins produced by spiders and scorpions. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see., e.g., the “AtTFDB” database listing the transcription factor families identified in the model plant Arabidopsis thaliana), publicly available at agris-knowledgebase [dot]org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast or fungal mating pheromones, invertebrate reproductive and larval signaling pheromones, see, e.g., Altstein (2004) Peptides, 25:1373-1376). Embodiments of agriculturally useful polypeptides confer a beneficial agronomic trait, e.g., herbicide tolerance, insect control, modified yield, increased fungal or oomycte disease resistance, increased virus resistance, increased nematode resistance, increased bacterial disease resistance, plant growth and development, modified starch production, modified oils production, high oil production, modified fatty acid content, high protein production, fruit ripening, enhanced animal and human nutrition, production of biopolymers, environmental stress resistance, pharmaceutical peptides and secretable peptides, improved processing traits, improved digestibility (e.g., reduced levels of toxins or reduced levels of compounds with “anti-nutritive” qualities such as lignins, lectins, and phytates), enzyme production, flavor, nitrogen fixation, hybrid seed production, fiber production, and biofuel production. Non-limiting examples of agriculturally useful polypeptides include polypeptides that confer herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; 5,763,241; 10,017,549; 10,233,217; 10,487,123; 10,494,408; 10,494,409; 10,611,806; 10,612,037; 10,669,317; 10,827,755; 11,254,950; 11,267,849; 11,130,965; 11,136,593; and 11,180,774), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; 5,958,745; and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700). In some embodiments, the RNA molecule further includes an internal ribosome entry site (IRES) located 5′ and immediately adjacent to the at least one coding sequence. In yet other embodiments, the cargo RNA molecule includes multiple coding sequences, and the RNA molecule further includes an IRES located 5′ and immediately adjacent to each of the coding sequences (e.g., open translational reading frames encoding a protein of interest. Useful IRES sequences include those depicted in Table 5.

In certain embodiments, the cargo RNA molecule includes a non-coding sequence. Such non-coding sequences include a hairpin RNA (hpRNA); an RNA that forms multiple stem-loops; an RNA pseudoknot; an RNA sequence that forms at least partially double-stranded RNA; a small interfering RNA (siRNA) or siRNA precursor; a microRNA (miRNA) or miRNA precursor; a ribozyme; a ligand-responsive ribozyme (aptazyme); an RNA aptamer; or a long noncoding RNA (lncRNA).

In some embodiments, the cargo RNA molecule comprises a CRISPR guide RNA. CRISPR-associated endonucleases such as Cas9, Cas12 and Cas13 endonucleases are used as genome editing tools in different plants; see, e.g., Wolter et al. (2019) BMC Plant Biol., 19:176-183); Aman et al. (2018) Genome Biol., 19:1-10. CRISPR/Cas9 requires a two-component crRNA: tracrRNA “guide RNA” (“gRNA”) that contains a targeting sequence (the “CRISPR RNA” or “crRNA” sequence) and a Cas9 nuclease-recruiting sequence (tracrRNA). Efficient Cas9 gene editing is also achieved with the use of a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991. Commercial manufacturers of CRISPR nucleases and guide RNAs provide algorithms for designing guide RNA sequences; see, e.g., guide design tools provided by Integrated DNA Technologies at www [dot]idtdna [dot]com/pages/products/crispr-genome-editing/alt-r-crispr-cas9-system. Some Cas nucleases, including Cas12a and Cas13, do not require a tracrRNA.

For many Cas nucleases, guide sequence designs are constrained by the requirement that the DNA target sequence (to which the crRNA is designed to be complementary) must be adjacent to a proto-spacer adjacent motif (“PAM”) sequence that is recognized by the specific Cas nuclease to be employed. Cas nucleases recognize specific PAM sequences and there is a diversity of nucleases and corresponding PAM sequences; see, e.g., Smakov et al. (2017) Nature Reviews Microbiol., doi: 10.1038/nrmicro.2016.184. For example, Cas9 nucleases cleave dsDNA, require a GC-rich PAM sequence located 3′ to the DNA target sequence to be targeted by the crRNA component of the guide RNA, and cleave leaving blunt ends. Cas 12a nucleases cleave dsDNA, require a T-rich PAM sequence located 5′ to the DNA target sequence to be targeted by the crRNA component of the guide RNA, and cleave leaving staggered ends with a 5′ overhang. Cas13 nucleases cleave single-stranded RNAs and do not require a PAM sequence; instead, Cas 13 nuclease are guided to their targets by a single crRNA with a direct repeat (“DR”). In practice, the crRNA component of a guide RNA is generally designed to have a length of between 17-24 nucleotides (frequently 19, 20, or 21 nucleotides) and exact complementarity (i.e., perfect base-pairing) to the targeted gene or nucleic acid sequence that is itself adjacent to a PAM motif (when required by the Cas nuclease). A crRNA component having less than 100% complementarity to the target sequence can be used (e.g., a crRNA with a length of 20 nucleotides and between 1-4 mismatches to the target sequence) but this increases the potential for off-target effects.

Non-limiting examples of effective guide design are found, e.g., in US Patent Application Publications US 2019/0032131, 2015/0082478, and 2019/0352655, which are each incorporated by reference in their entirety herein. For the purposes of gene editing, CRISPR “arrays” can be designed to include one or multiple guide RNA sequences corresponding to one or more desired target DNA sequence(s); see, for example, Cong et al. (2013) Science, 339:819-823; Ran et al. (2013) Nature Protocols, 8:2281-2308.

In an embodiment, a cargo RNA molecule that is integrated into a polynucleotide includes at least one CRISPR guide RNA; release of the guide RNA is mediated, e.g., by flanking DR sequences, ribozyme sequences, or other self-cleaving or trans-cleaving RNAs, or by cleavage by an endogenous ribonuclease. The corresponding Cas nuclease can be provided by separate or concurrent delivery, e.g., by co-delivery with a vector or polynucleotide, or by transient or stable expression of the corresponding Cas nuclease in the cell to which the polynucleotide is delivered.

In certain embodiments, the 5′ RNA replication element includes a 5′ UTR element of a partitivirus genome (e.g., either a CP or RDRP PV genome including a PV 5′ UTR set forth in Table 1). In embodiments, the 5′ RNA replication element further includes a genomic sequence of the partitivirus that is natively located 3′ to and optionally adjacent or immediately adjacent to the 5′ UTR sequence. In embodiments, the 3′ RNA replication element includes a 3′ UTR sequence of a Partitivirus genome (e.g., either a CP or RDRP PV genome including a PV 3′ UTR set forth in Table 1). In embodiments, the 3′ RNA replication element further includes a genomic sequence of the partitivirus that is natively located 5′ to and optionally adjacent or immediately adjacent to the 3′ UTR sequence.

In other embodiments, the RNA molecule further includes at least one RNA molecule encoding a viral MP. In certain embodiments, the at least one RNA molecule encoding an MP is located (a) before the cargo RNA molecule, (b) after the cargo RNA molecule, or (c) both before and after the cargo RNA molecule. In embodiments, the at least one RNA sequence encoding an MP includes at least two RNA sequences encoding different MPs or a single RNA sequence encoding multiple copies of MPs.

In some embodiments, the recombinant DNA molecule further includes a discrete expression cassette including a second promoter that is functional in the cell and is operably linked to a DNA sequence encoding at least one viral movement protein, and optionally a terminator element.

In some embodiments, the RNA molecule further includes an ERE, where the ERE is located close to or adjacent to the 3′ RNA replication element, and optionally wherein the 3′ RNA replication element includes a 3′ UTR sequence of the partitivirus. In embodiments, the ERE includes a viral OAS such as a tobacco mosaic virus OAS (TMV-OAS) or an OAS set forth in Table 2.

In embodiments, the RNA molecule further includes at least one tRNA-like sequence (TLS), and wherein the at least one tRNA-like sequence includes a tRNA-like sequence from an Arabidopsis FT mRNA (e.g. a TLS in an Arabidopsis FT mRNA of Table 4). In some embodiments, the RNA molecule includes a tRNA-like sequence selected from SEQ ID NOs: 184-231. In some embodiments, the RNA molecule includes a modified tRNA-like sequence that has at least 90% sequence identity to a scaffold tRNA-like sequence selected from the group consisting of SEQ ID NOs: 184-231 and that maintains the secondary structure of the scaffold tRNA-like sequence. In still other embodiments, the RNA molecule further includes at least one RNA encoding a viral MP, a tRNA-like sequence from an Arabidopsis FT mRNA, and an encapsidation recognition sequence including TMV-OAS.

In some embodiments, the cargo RNA molecule is up to about 3.2 kb in length. In embodiments, the cargo RNA molecule includes: (a) at least one coding sequence, (b) at least one non-coding sequence, or (c) both at least one coding sequence and at least one non-coding sequence. In other embodiments, the cargo RNA molecule includes at least one coding sequence, and wherein the RNA molecule further includes an internal ribosome entry site (IRES) located 5′ and immediately adjacent to the at least one coding sequence. In other embodiments, the cargo RNA molecule includes multiple coding sequences, and wherein the RNA molecule further includes an IRES located 5′ and immediately adjacent to each of the coding sequences.

In other embodiments, the cargo RNA molecule includes at least one non-coding sequence, and wherein the at least one non-coding sequence is selected from the group consisting of a hairpin RNA (hpRNA); an RNA that forms multiple stem-loops; an RNA pseudoknot; an RNA sequence that forms at least partially double-stranded RNA; a small interfering RNA (siRNA) or siRNA precursor; a microRNA (miRNA) or miRNA precursor; a ribozyme; a ligand-responsive ribozyme (aptazyme); an RNA aptamer; and a long noncoding RNA (lncRNA).

In embodiments, a DNA sequence encoding at least one ribozyme is provided. In embodiments, the at least one ribozyme is located 5′ to the 5′ RNA replication element or 3′ to the 3′ RNA replication element. In embodiments, a DNA sequence encoding at least one ligand-responsive ribozyme (aptazyme) is provided. In embodiments, the at least one ligand-responsive ribozyme is located 5′ to the 5′ RNA replication element or 3′ to the 3′ RNA replication element.

Recombinant RNA molecules comprising the aforementioned or otherwise disclosed 5′ RNA replication elements, a cargo RNA molecule(s), and 3′ RNA replication elements, as well as additional aforementioned or otherwise disclosed elements are also provided herein. In certain embodiments, the recombinant RNA molecules are produced by a recombinant DNA molecule provided herein. In certain embodiments, the recombinant RNA molecules are produced by an in vivo or in vitro (e.g., cell free) RNA replication process through the action of a RDRP or an RNA Promoter Dependent RNA polymerase (RPDRP).

Expression systems comprising the recombinant polynucleotides are also provided. Such expression systems include both cell-based and cell free expression systems. In certain embodiments, cell-free expression system can include (a) an RNA molecule comprising, in 5′ to 3′ order: (i) a 5′ RNA replication element; (ii) a cargo RNA molecule; and (iii) a 3′ RNA replication element; and, optionally, further comprising at least one additional RNA or other element. Such additional RNA elements can include at least one RNA encoding a viral MP, at least one tRNA-like sequence, an OAS, an RDRP protein that recognizes the 5′ and 3′ RNA replication elements, an RNA promoter, and/or an RPDRP. In some embodiments, the RDRP protein is provided by a partitivirus, e.g. a partitivirus that is endogenous to a cell in which expression is desired. In certain embodiments, cell-based expression system can include (a) a recombinant DNA molecule including a heterologous promoter that is functional in a cell and is operably linked to a DNA sequence encoding an RNA molecule comprising, in 5′ to 3′ order: (i) a 5′ RNA replication element; (ii) a cargo RNA molecule; and (iii) a 3′ RNA replication element; and, optionally, further comprising at least one additional RNA or other element. Such additional RNA elements can include at least one RNA encoding a viral MP, at least one tRNA-like sequence, an OAS, an RDRP protein that recognizes the 5′ and 3′ RNA replication elements, an RNA promoter, and/or an RPDRP. In certain embodiments, cell-based expression system can include (a) a recombinant RNA molecule comprising, in 5′ to 3′ order: (i) a 5′ RNA replication element; (ii) a cargo RNA molecule; and (iii) a 3′ RNA replication element; and, optionally, further comprising at least one additional RNA or other element. Such additional RNA elements can include at least one RNA encoding a viral MP, at least one tRNA-like sequence, an OAS, an RDRP protein that recognizes the 5′ and 3′ RNA replication elements, an RNA promoter, and/or an RPDRP. In some embodiments, the RDRP protein is provided by a partitivirus. In still other embodiments, the 5′ RNA replication element and the 3′ RNA replication element are obtained from the same partitivirus and/or from the same Partitivirus genome (e.g., both obtained from the same PV capsid genome or both obtained from the same PV RDRP genome) or from related partitivirus genomes having at least 85%, 90%, 95%, 98%, or 99% sequence identity to one another. In some embodiments, a cell used in the expression system is a bacterial cell, a plant cell, a fungal cell, or an animal cell (e.g., an insect cell). In embodiments, a cell used in the expression system endogenously contains a partitivirus having a genome that encodes an RDRP that recognizes the 5′ and 3′ RNA replication elements. In some embodiments, the expression system further includes a viral capsid protein that is recognized by the encapsidation recognition element and encapsidates the RNA molecule. In some embodiments, the viral capsid protein is: (a) expressed by the recombinant DNA molecule in the cell (e.g., where the recombinant DNA molecule further includes a discrete expression cassette comprising a second promoter operably linked to a DNA sequence encoding the viral capsid protein, and optionally a terminator element), (b) co-expressed by a second recombinant DNA molecule in the cell; (c) provided exogenously to the cell; or (d) expressed by a virus in the cell. In embodiments, the RDRP protein is heterologous to the cell. In embodiments, the RDRP protein is provided exogenously to the cell. In certain embodiments, the RDRP protein that recognizes the 5′ and 3′ RNA replication elements is endogenously expressed in the plant cell by the partitivirus virus (e.g., where the partitivirus virus occurs naturally in the plant cell). In embodiments, the partitivirus is native to or endemic to the plant cell. In embodiments, the partitivirus that is endemic to the plant cell is non-pathogenic. In embodiments, the partitivirus that is endemic to the plant cell is non-pathogenic and commensal. In embodiments, the partitivirus is an exogenously introduced partitivirus (i.e., not endemic or native to the host, but artificially introduced). For example, a partitivirus natively found in one plant species, variety, or germplasm can be introduced, with or without a corresponding recombinant partitiviral satellite RNA, into a different plant species, variety, or germplasm. In embodiments, a complete self-replicating partitiviral satellite system is introduced into a plant or plant cells, wherein the self-replicating partitiviral satellite system includes: (1) a recombinant partitiviral satellite RNA comprising, from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP; wherein the 5′ RNA replication element, the cargo RNA molecule, and the 3′ RNA replication element are operably linked, and wherein the promoter and the cargo RNA molecule are heterologous to the 5′ RNA replication element and the 3′ RNA replication element; and (2) an exogenous partitivirus (e.g., a partitivirus that is not endemic or native to the plant or plant cells) that is capable of replication in the plant or plant cells and that encodes the partitiviral RDRP that recognizes the 5′ and 3′ replicase recognition sequences in the recombinant partitiviral satellite RNA. In embodiments, the partitiviral RDRP comprises a protein having at least 85%, 90%, 95%, 97%, or 99% sequence identity to SEQ ID NO: 272-290, 384, or 390. In embodiments, the recombinant DNA molecule or recombinant RNA molecule further comprises at least one RNA encoding a viral MP, a tRNA-like sequence from an Arabidopsis FT mRNA, and an encapsidation recognition sequence including a TMV-OAS.

Cells comprising any of the aforementioned or otherwise disclosed recombinant polynucleotides (e.g., recombinant DNAs, recombinant RNAs, or vectors comprising or encoding the same) are provided herein. Cells comprising the recombinant polynucleotides include prokaryotic (e.g., a bacterium, such as a bacterium capable of transforming a eukaryotic cell) or eukaryotic (e.g., a plant cell, fungal cell, or animal cell such as an insect cell) cells. In certain embodiments, the cells are bacterial cells capable of transforming a plant cell (e.g., an Agrobacterium sp., a Sinorhizobium sp., a Mesorhizobium sp., Bradyrhizobium sp., Rhizobium sp., or an Ensifer sp. cell. Bacterial cells capable of transforming a plant cell suitable for use with the recombinant polynucleotides provided herein include Agrobacterium sp., a Sinorhizobium sp., a Mesorhizobium sp., Bradyrhizobium sp., Rhizobium sp., or an Ensifer sp. cell are disclosed in US patent application publications US20170369898 and US20180312854, each incorporated herein by reference in their entireties.

Vectors suitable for maintenance, propagation, and/or expression of the recombinant polynucleotides in the aforementioned prokaryotic or eukaryotic cells are also provided herein. Such vectors can comprise any of the aforementioned or otherwise disclosed recombinant polynucleotides, recombinant DNA molecules, and recombinant RNA molecules as well as those polynucleotides molecules described in the Examples. In some embodiments, the bacterium that mediates the plant transformation is an Agrobacterium sp., a Sinorhizobium sp., a Mesorhizobium sp., Bradyrhizobium sp., Rhizobium sp., or an Ensifer sp. In embodiments where an Agrobacterium sp. is used, the vector includes T-DNAs flanking the recombinant DNA molecule encoding the recombinant RNA molecule (e.g., as described in US patent application publications US20170369898 and US20180312854, each incorporated herein by reference in their entireties). In some embodiments, the vector is contained within a plant cell or within a bacterial cell (Agrobacterium sp., a Sinorhizobium sp., a Mesorhizobium sp., Bradyrhizobium sp., Rhizobium sp., or an Ensifer sp. cell).

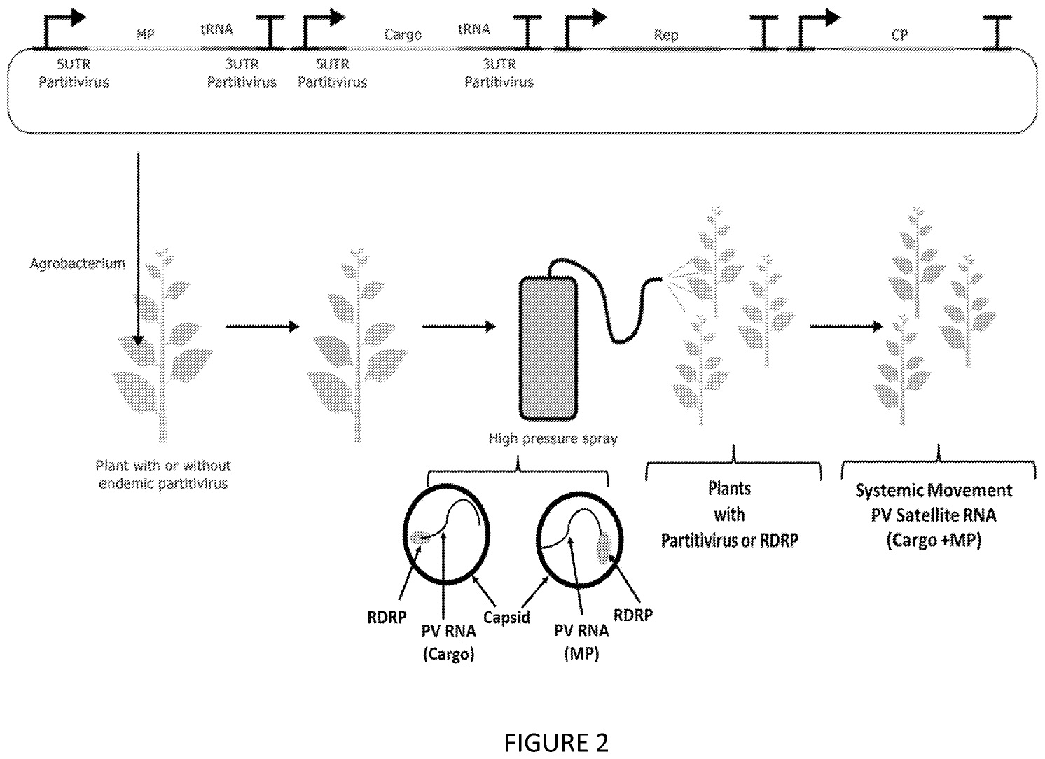

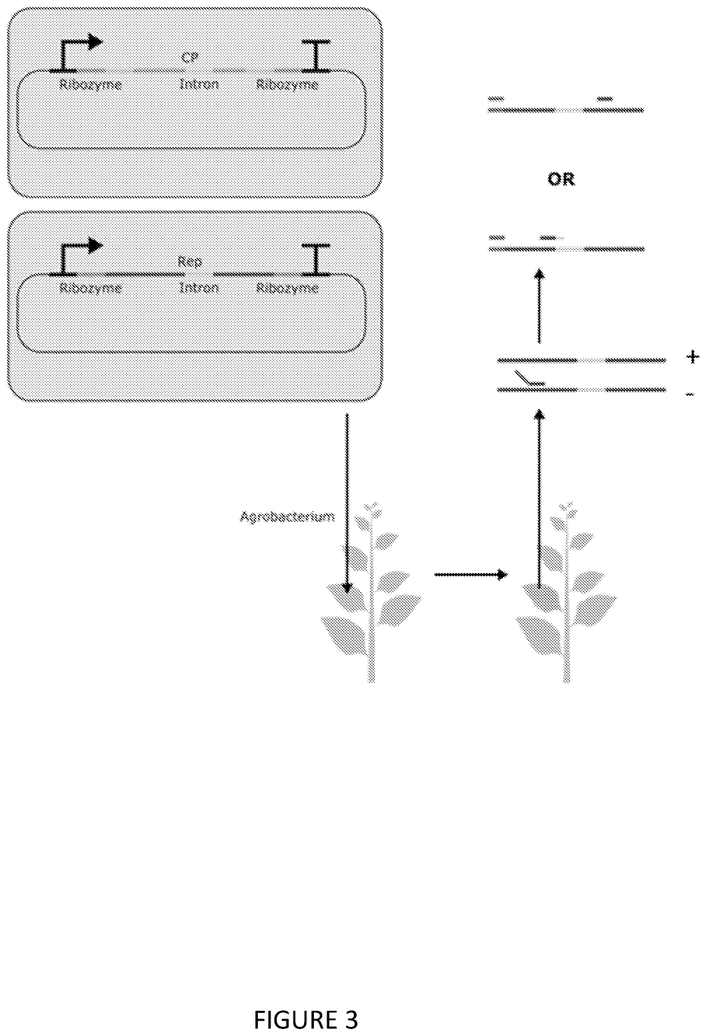

Viral particles comprising any of the aforementioned or otherwise disclosed recombinant RNA molecules are also provided. In one embodiment, FIG. 2 shows infiltration of a host or production plant using Agrobacterium-mediated transformation with a polynucleotide comprising (5′ to 3′): (i) a promoter which is operably linked to a viral MP coding sequence and a TLS element flanked by partitiviral 5′ and 3′ replication elements; (ii) a promoter which is operably linked to a cargo RNA molecule and a TLS element flanked by partitiviral 5′ and 3′ replication elements; (iii) a promoter operable linked to a Rep (RDRP) coding sequence; and (iv) a promoter operably linked to a CP encoding sequence. Heterologous promoters independently drive expression of the capsid protein and the cargo as depicted. The RNA expressed from the polynucleotide includes an OAS. A host plant is transformed for production of the synthetic partitiviral satellite RNA and satellite particles comprising the encapsidated RNA. The expressed and encapsidated partitiviral satellite RNA is subsequently isolated from leaf material or other tissue of the host plant, purified (and, if desired, formulated) for high pressure spraying onto plants that endogenously contain the corresponding partitivirus or a recombinant source of the PV RDRP for subsequent expression and replication of the partitiviral satellite RNA and satellite particles comprising the same in encapsidated form. In certain embodiments, spraying with the encapsidated satellite particles with certain cargo RNA molecules can be used to modify the plant as desired. Presence of movement protein and t-RNA like sequences facilitates systemic movement throughout the plant receiving the high-pressure spray satellite particles comprising the desired recombinant RNA molecules comprising the encoded MP and cargo RNA. The scheme depicted in FIG. 2 can be adapted to any of the other embodiments disclosed herein. In certain embodiments, plants without a systemic partitivirus which provides the PV RDRP can further comprise a recombinant DNA or RNA molecule which encodes and provides the RDRP.

Target plants and plant cells used as hosts for synthetic partitiviral satellite RNAs (e.g., recombinant RNAs) provided herein include both monocot and dicot plants and plant cells which can support partitivirus replication which can include row crop plants, fruit-producing plants and trees, vegetables, trees, and ornamental plants including ornamental flowers, shrubs, trees, groundcovers, and turf grasses. In certain embodiments, the target plants and plant cells hosts for synthetic partitiviral satellite RNAs include alfalfa, beet, Brassica spp. (e.g., canola), Cannabis spp., carrot, citrus, clover, curry, dill, Raphanus, fig, Fragaria, hop, maize, melon, persimmon, pepper, Primula spp., Sinapis, spinach, tomato, and Vicia plants and plant cells. In certain embodiments, target plants and plant cells used as hosts for synthetic partitiviral satellite RNAs (e.g., recombinant RNAs) provided herein alfalfa, beet, Brassica spp. (e.g., canola), Cannabis spp., carrot, citrus, clover, curry, dill, Raphanus, fig, Fragaria, hop, maize, melon, persimmon, pepper, Primula spp., Sinapis, spinach, tomato, and Vicia plants and plant cells. In certain embodiments, the dicot plant or plant cell is a member of the genus Brassica, Capsicum, Cucumis, Cucurbita, Hordeum, Gossypium, Nicotiana, Solanum, or Glycine. In certain embodiments, the dicot target plants and plant cells hosts for synthetic partitiviral satellite RNAs (e.g., recombinant RNAs) include avocado (Persea americana), broad bean (Vicia faba), canola (Brassica napus), calabash (Lagenaria siceraria), common bean (Phaseolus vulgaris), cotton (Gossypium hirsutum), cucumber (Cucumis sativus), guar (Cyamopsis tetragonoloba), Malabar spinach (Basella alba), melon (Cucumis melo), pepper (Capsicum frutescens, Capsicum annuum), potato (Solanum tuberosum), pumpkin (Cucurbita pepo), soybean (Glycine max), tomato (Solanum lycopersicum), wax gourd (Benincasa hispida), winged bean (Psophocarpus tetragonolobus), and yerba mate (Ilex paraguariensis) plants and plant cells. In certain embodiments, the monocot plant or plant cells is a member of the genus Avena, Hordeum, Oryza, Secale, Triticum, Sorghum, or Zea. In certain embodiments, monocot target plants and plant cells used as hosts for synthetic partitiviral satellite RNAs (e.g., recombinant RNAs) provided herein include oats (Avena sativa), barley (Hordeum vulgare), rice (Oryza sativa, Oryza rufipogon), rye (Secale cereale), wheat (Triticum aestivum), sorghum (Sorghum bicolor), and maize (Zea mays) plants and plant cells.

Methods of using or use of any of the aforementioned or otherwise disclosed recombinant polynucleotides, expression systems, cells, and/or vectors to: (i) provide a synthetic partitiviral satellite RNA to a plant cell, (ii) obtain a phenotypic change in a plant or plant cell; (ii) increase a plant's resistance of a pest or pathogen, (iii) increase a plant's resistance to stress, (iv) express a polypeptide in a plant or plant cell, and/or (v) manufacture a synthetic partitiviral virus particle are also provided. In certain embodiments of the methods, the recombinant RNA molecule or a formulation thereof is provided by contacting the plant or plant cell with the RNA or formulation thereof. In other embodiments, the recombinant RNA molecule is provided by expressing in the plant or plant cell a DNA molecule that encodes the recombinant RNA molecule or a formulation. In other embodiments, the recombinant RNA molecule is provided by contacting the plant or plant cell with cells which comprise a DNA molecule that encodes the recombinant RNA molecule and are capable of transforming the plant or plant cell. In other embodiments, the recombinant RNA molecule is provided by contacting the plant or plant cell with a satellite particle comprising an encapsidated recombinant RNA molecule or a formulation thereof. In embodiments, the 5′ RNA replication element has a nucleotide sequence obtained or derived from a partitivirus genomic sequence; and/or the 3′ RNA replication element has a nucleotide sequence obtained or derived from a Partitivirus genomic sequence. In certain embodiments, the plant cell includes the partitivirus virus, and the RDRP protein is provided to the plant cell by the partitivirus virus. In embodiments, the partitivirus is endemic to the plant cell. In embodiments, the partitivirus that is endemic to the plant cell is non-pathogenic and/or commensal to the plant cell. In other embodiments, the partitivirus is exogenously provided to the plant cell. In some embodiments, the RDRP protein is exogenously provided to the plant cell. In embodiments, the recombinant RNA molecule is produced in a fermentation system. In embodiments, the recombinant RNA molecule is provided to the plant cell by transcribing in the plant cell a recombinant DNA construct including a promoter functional in the plant cell and operably linked to a DNA sequence encoding the recombinant RNA molecule. In embodiments, the recombinant RNA molecule further includes an encapsidation recognition element, and the plant cell further includes a viral capsid protein capable of encapsidating the synthetic partitiviral satellite RNA. In embodiments, wherein the viral capsid protein is exogenously provided to the plant cell. In other embodiments, the recombinant DNA construct further includes a DNA sequence encoding a viral capsid protein. Still in other embodiments, the recombinant DNA construct further includes a second promoter functional in the plant cell and operably linked to the DNA sequence encoding the viral capsid protein. In embodiments, the viral capsid protein is expressed in the plant cell and encapsidates the synthetic partitiviral satellite RNA. In yet other embodiments, the plant cell includes the partitivirus, and the partitivirus provides to the plant cell: (a) the RDRP protein, (b) the viral capsid protein, or (c) both the RDRP protein and the viral capsid protein. In certain embodiments, the methods can further comprise a first step of providing a population of plants comprising the plant cells comprising: (i) the partitivirus which provides the RDRP; or (ii) recombinant polynucleotide molecule that encodes the RDRP; and then providing the recombinant RNA molecule to the plants comprising the plant cells. In certain embodiments, the methods can further comprise the step of determining if the plant cell comprises a partitivirus which can provide the RDRP. In embodiments wherein it is determined that the plant cell comprises the partitivirus which can provide the RDRP, the partitivirus, the RDRP protein, and/or the recombinant polynucleotide encoding the RDRP is optionally not exogenously provided to the plant cell. In other embodiments wherein it is determined that the plant cell does not comprise the partitivirus which can provide the RDRP and the partitivirus is exogenously provided to the cell, the RDRP protein or the recombinant polynucleotide encoding the RDRP is exogenously provided to the plant cell, or a combination of the partitivirus, RDRP protein, or polynucleotide encoding the RDRP is exogenously provided to the plant cell. In embodiments, a complete self-replicating partitiviral satellite system is introduced into a plant or plant cells, wherein the self-replicating partitiviral satellite system includes: (1) a recombinant partitiviral satellite RNA comprising, from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP; wherein the 5′ RNA replication element, the cargo RNA molecule, and the 3′ RNA replication element are operably linked, and wherein the promoter and the cargo RNA molecule are heterologous to the 5′ RNA replication element and the 3′ RNA replication element; and (2) an exogenous partitivirus (e.g., a partitivirus that is not endemic or native to the plant or plant cells) that is capable of replication in the plant or plant cells and that encodes the partitiviral RDRP that recognizes the 5′ and 3′ replicase recognition sequences in the recombinant partitiviral satellite RNA. In embodiments, the partitiviral RDRP comprises a protein having at least 85%, 90%, 95%, 97%, or 99% sequence identity to SEQ ID NO: 272-290, 384, or 390. The presence or absence of a partitivirus in a target plant can be determined by an RNA detection assay (e.g., an RT-PCR assay) using nucleic acid probes and/or primers which can detect any part of a Partitivirus genome including a 5′ RNA replication element, a CP and/or RDRP coding region, and/or a 3′ RNA replication element. Such probes and primers include those which detect any of the 5′ or 3′ RNA replication elements set forth in Table 1 or having significant sequence identity thereto (e.g., at least about 80%, 85%, 90%, 95%, 98%, or 99% of a length of at least about 18, 20, 30, 40 or 50 nt). The presence or absence of a partitivirus in a target plant can be determined by a protein detection assay (e.g., an immunoassay) directed to a PV CP or RDRP (e.g., a CP or RDRP encoded by or homologous to a CP or RDRP encoded by a PV genome disclosed in Table 1). Target plants and plant cells used in the methods include all aforementioned target plants and plant cell hosts for synthetic partitiviral satellite RNAs (e.g., recombinant RNAs). In certain embodiments, the target plants and plant cell hosts used in the methods include alfalfa, beet, brassica (e.g., canola), cannabis, carrot, citrus, clover, curry, dill, Raphanus, fig, Fragaria, hop, maize, melon, persimmon, pepper, primula, Sinapis, spinach, tomato, and Vicia plants.

In certain embodiments of any of the aforementioned or otherwise disclosed methods, the recombinant RNA that effects: (i) a phenotypic change in the plant or plant cell; (ii) increases a plant's resistance to a pest or pathogen; or (iii) increases a plant's resistance to stress can include an RNA for modulating a target gene's expression relative to the target gene's expression in a control plant or plant cell not provided with the recombinant RNA molecule, and the phenotypic change, increased resistance to the pest or pathogen, or increased resistance to stress is a result of the modulation. In embodiments, the modulation is (a) an increase of the target gene's expression; or (b) a decrease of the target gene's expression. In some embodiments, expression of the target gene is increased by up to about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a control plant or plant cell lacking the recombinant RNA). In certain embodiments, expression of the target gene is increased by up to about 2-, 3-, 4-, 5-, 6-, 8-, 9-, 10-fold, or more relative to a reference level (e.g., a level found in a control plant or plant cell lacking the recombinant RNA molecule). In some embodiments, expression of the target gene is decreased by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a level found in a control plant or plant cell lacking the recombinant RNA). In certain embodiments, expression of the target gene is decreased by up to about 2-, 3-, 4-, 5-, 6-, 8-, 9-, 10-fold, or more relative to a reference level (e.g., a level found in a control plant or plant cell lacking the recombinant RNA molecule). Modulation of target gene expression can be effected by one or more of an RNA for modifying the genome, the epigenome, and/or transcriptome of the plant or plant cell. RNAs for modifying the genome can include gRNAs recognized by CAS nucleases, RNAs encoding TALENs or artificial zinc finger proteins (aZFN). RNAs for modifying the epigenome can include RNAs which provide RNA directed DNA methylation such as in promoter regions of target genes (Matzke and Mosher (2014). doi: 10.1038/nrg3683). An RNA for modifying the transcriptome can comprise a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor a phased siRNA or phased siRNA precursor; a ribozyme; a ligand-responsive ribozyme (aptazyme); an RNA aptamer; or a long noncoding RNA (lncRNA).

In any of the aforementioned or otherwise disclosed methods wherein a phenotypic change is obtained or effected in a plant or plant cell, the cargo RNA molecule can comprise an RNA that effects a phenotypic change in the plant or plant cell in comparison to a plant or plant cell lacking the recombinant RNA. In certain embodiments, phenotypes that are changed include developmental rate, growth rate, size, yield (e.g., intrinsic yield), vigor, photosynthetic capability, flavor, starch production, protein content, carbohydrate content, oil content, fatty acid content, lipid content, digestibility, biomass, shoot length, root length, root architecture, seed set, seed weight, seed quality (e.g., nutritional content), germination, fruit set, rate of fruit ripening, production of biopolymers, production of fibers, production of biofuels, production of pharmaceutical peptides, production of secretable peptides, enzyme production, improved processing traits, or amount of harvestable produce. In some embodiments, phenotypes that are changed include taste, appearance, or shelf-life of a product harvested from the plant. In other embodiments, phenotypes that are changed include flower size, flower color, flower patterning, flower morphology including presence or absence of stamens, flower number, flower longevity, flower fragrance, leaf size, leaf color, leaf patterning, leaf morphology, plant height, or plant architecture.

In any of the aforementioned or otherwise disclosed methods wherein a plant's resistance to a pest or pathogen is increased, the recombinant RNA can comprise an RNA that inhibits expression of a gene of the pest or pathogen and/or inhibits replication of the genome of the pest or pathogen. In certain embodiments, the pest or pathogen is selected from the group comprising: a bacterium, a virus other than a partitivirus, a fungus, an oomycete, and an invertebrate (e.g., an arthropod or a nematode). Target viruses other than a partitivirus include; (i) positive-strand RNA viruses in the Bromoviridae, Closteroviridae, Luteoviridae, or Potyviridae family; (ii) negative strand RNA viruses in the Bunyaviridae and Rhabdoviridae family; (iii) dsDNA viruses in the family Caulimoviridae; and (iii) ssDNA viruses in the family Geminiviridae. Target arthropods pests include coleopteran and lepidopteran insects. Target fungal pathogens include Magnaporthe spp., Botrytis spp., Puccinia spp.; Fusarium spp., Blumeria spp., Mycosphaerella spp., Colletotrichum spp., Ustilago spp., Melampsora spp., Phakopsora spp., Phytophthora spp., and Rhizoctonia spp. In embodiments, the cargo RNA molecule effects an increase in the plant's resistance to a pest or pathogen, relative to that in a plant not provided with the recombinant RNA molecule.

In any of the aforementioned or otherwise disclosed methods wherein a plant's resistance to stress is increased, the recombinant RNA can comprise an RNA that targets a plant gene which provides such resistance. In embodiments, the RNA that effects an increase in the plant's resistance to stress in the plant or plant cell includes an RNA for modulating the target gene's expression relative to the target gene's expression in a control plant or plant cell not provided with the recombinant RNA molecule, and wherein the increase stress resistance is a result of the modulation. In embodiments, the modulation is (a) an increase of the target gene's expression; or (b) a decrease of the target gene's expression. In still other embodiments, the RNA that effects an increase in the plant's resistance to stress in the plant or plant cell comprises a messenger RNA encoding a protein which confers the stress resistance. In still other embodiments, the messenger RNA includes an RNA sequence absent in the transcriptome of the plant or plant cell lacking the recombinant RNA. In embodiments, the stress includes at least one abiotic stress selected from the group including: nutrient stress, light stress, drought stress, heat stress, and cold stress. In other embodiments, the stress includes at least one biotic stress selected from the group including: crowding, shading, and allelopathy (e.g., resulting from allelopathic chemicals including a juglone produced by walnut trees).

In any of the aforementioned or otherwise disclosed methods wherein an exogenous polypeptide (e.g., an exogenous polypeptide) is expressed in a plant or plant cell, the cargo RNA can encode the exogenous polypeptide. In embodiments, the polypeptide is isolated (e.g., separated from at least one other cellular components such as a carbohydrate, a lipid, or another protein) or polypeptide is purified.

In any of the aforementioned or otherwise disclosed methods wherein a synthetic partitiviral satellite particle is manufactured, such manufacture can occur in either a cell-based system or a cell-free system. Cell-based methods of manufacturing a synthetic partitiviral satellite particle can comprise: (a) providing to a plant cell the recombinant RNA molecule, wherein the recombinant RNA molecule comprises an encapsidation recognition element (ERE), and wherein the plant cell comprises an RDRP protein that recognizes the 5′ RNA replication element and 3′ RNA replication element catalyzes synthesis of a synthetic partitiviral satellite RNA from the recombinant RNA molecule and a viral capsid protein, wherein the ERE provides for encapsidation of the RNA by the viral capsid protein; and optionally (b) isolating the synthetic partitiviral satellite particle from the plant cell, a plant comprising the plant cell, or from media in which the plant cell or the plant had been grown. Cell-free methods of manufacturing a synthetic partitiviral satellite particle include methods where the recombinant RNA molecule is combined with a viral capsid protein in a vessel, wherein the recombinant RNA molecule comprises an encapsidation recognition element (ERE), and wherein the ERE provides for encapsidation of the RNA by the viral capsid protein in the vessel; optionally wherein the method further comprises isolating the synthetic partitiviral satellite particle from uncombined RNA and/or viral capsid protein in the vessel. In certain embodiments of either cell-based or cell-free methods, the synthetic satellite particle is isolated (e.g., separated from at least one other cellular components such as an organelle, a membrane, a carbohydrate, a lipid, or another protein) or is purified. In certain embodiments, the methods can further comprise formulating the synthetic partitiviral satellite particle.

Synthetic partitiviral satellite particles comprising the recombinant RNA, including those made by the aforementioned methods are also provided. Methods of providing any of the aforementioned synthetic partitiviral satellite particles to a plant, including contacting (e.g., spraying, dusting, injecting, soaking, etc.) the plant with the synthetic partitiviral satellite particle or a formulation thereof are also provided.

The recombinant polynucleotides, cells comprising the same, and synthetic partitiviral satellite particles described herein can be formulated either in pure form (e.g., the composition contains only the recombinant polynucleotide) or together with one or more additional formulation components to facilitate application or delivery of the compositions. In embodiments, the additional formulation component includes, e.g., a carrier (i.e., a component that has an active role in delivering the active agent (e.g., recombinant polynucleotide); for example, a carrier can encapsulate, covalently or non-covalently modify, or otherwise associate with the active agent in a manner that improves delivery of the active agent) or an excipient (e.g., a delivery vehicle, adjuvant, diluent, surfactant, stabilizer, or tonicity agent). In some embodiments, the composition is formulated for delivery to a plant.

In some aspects, the disclosure provides a formulation comprising any of the compositions described herein. In some embodiments, the formulation is a liquid, a gel, or a powder. In some embodiments, the formulation is configured to be sprayed on plants, to be injected into plants (e.g., into the vascular system of a plant), to be rubbed on leaves, to be soaked into plants, to be coated onto plants, or be coated on seeds, or to be delivered through root uptake (e.g., in a hydroponic system or via soil).

Depending on the intended objectives and prevailing circumstances, the composition can be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can.

In some instances, the recombinant polynucleotide makes up about 0.1% to about 100% of the composition, such as any one of about 0.01% to about 100%, about 1% to about 99.9%, about 0.1% to about 10%, about 1% to about 25%, about 10% to about 50%, about 50% to about 99%, or about 0.1% to about 90% of active ingredients (e.g., recombinant polynucleotides). In some instances, the composition includes at least any of 0.1%, 0.5%, 1%, 5%, 10%, 20%, 30%, 40%, 50%, 60%, 70%, 80%, 90%, 95%, or more active ingredients (e.g., recombinant polynucleotides). In some instances, the concentrated agents are preferred as commercial products, the final user normally uses diluted agents, which have a substantially lower concentration of active ingredient.

In some embodiments, the composition is formulated for topical delivery to a plant. In some embodiments, the topical delivery is spraying, leaf rubbing, soaking, coating (e.g., coating using micro-particulates or nano-particulates), or delivery through root uptake (e.g., delivery in a hydroponic system).

In some embodiments, the composition further comprises a carrier and/or an excipient. In other embodiments, the composition does not comprise a carrier or excipient, e.g., comprises a naked polynucleotide (e.g., a naked RNA).

In some embodiments, the recombinant polynucleotide is delivered at a concentration of at least 0.1 grams per acre, e.g., at least 0.1, 1, 2, 5, 10, 15, 20, 25, 30, 35, 40, 45, or 50 grams per acre. In some embodiments, less than 120 liters per acre is delivered, e.g., less than 110, 100, 90, 80, 70, 60, 50, 40, 30, 20, 15, 10, 5, or 2 liters per acre or less than 1 liter per acre.

In some aspects, the formulation comprises a carrier. In some embodiments the formulation is an emulsion or a reverse emulsion, a liquid, or a gel. In embodiments, the formulation includes a carrier that serves as a physical support (e.g., solid or semi-solid surfaces or matrices, powders, or particles or nanoparticles). In embodiments, the active agent is encapsulated or enclosed in or attached to or complexed with a carrier including a liposome, vesicle, micelle, or other fluid compartment. In embodiments, the active agent is encapsulated or enclosed in or attached to or complexed with a carrier including a naturally occurring or synthetic, branched or linear polymer (e.g., pectin, agarose, chitin, chitosan, DEAE-dextran, polyvinylpyrrolidone (“PVP”), or polyethylenimine (‘PEI”)). In embodiments the carrier includes cations or a cationic charge, such as cationic liposomes or cationic polymers such as polyamines (e.g., spermine, spermidine, putrescine). In embodiments, the carrier includes a polypeptide such as an enzyme, (e.g., cellulase, pectolyase, maceroenzyme, pectinase), a cell penetrating or pore-forming peptide (e.g., poly-lysine, poly-arginine, or polyhomoarginine peptides).

Non-limiting examples of carriers include cationic liposomes and polymer nanoparticles reviewed by Zhang et al. (2007) J. Controlled Release, 123:1-10, and the cross-linked multilamellar liposomes described in US Patent Application Publication 2014/0356414 A1, incorporated by reference in its entirety herein. In embodiments, the carrier includes a nanomaterial, such as carbon or silica nanoparticles, carbon nanotubes, carbon nanofibers, or carbon quantum dots. Non-limiting examples of carriers include particles or nanoparticles (e.g., particles or nanoparticles made of materials such as carbon, silicon, silicon carbide, gold, tungsten, polymers, or ceramics) in various size ranges and shapes, magnetic particles or nanoparticles (e.g., silenceMag Magnetotransfection™ agent, OZ Biosciences, San Diego, CA), abrasive or scarifying agents, needles or microneedles, matrices, and grids.

In certain embodiments, particulates and nanoparticulates are useful in delivery of the polynucleotide composition or the nuclease or both. Useful particulates and nanoparticles include those made of metals (e.g., gold, silver, tungsten, iron, cerium), ceramics (e.g., aluminum oxide, silicon carbide, silicon nitride, tungsten carbide), polymers (e.g., polystyrene, polydiacetylene, and poly(3,4-ethylenedioxythiophene) hydrate), semiconductors (e.g., quantum dots), silicon (e.g., silicon carbide), carbon (e.g., graphite, graphene, graphene oxide, or carbon nanosheets, nanocomplexes, or nanotubes), and composites (e.g., polyvinylcarbazole/graphene, polystyrene/graphene, platinum/graphene, palladium/graphene nanocomposites). In certain embodiments, such particulates and nanoparticulates are further covalently or non-covalently functionalized, or further include modifiers or cross-linked materials such as polymers (e.g., linear or branched polyethylenimine, poly-lysine), polynucleotides (e.g., DNA or RNA), polysaccharides, lipids, polyglycols (e.g., polyethylene glycol, thiolated polyethylene glycol), polypeptides or proteins, and detectable labels (e.g., a fluorophore, an antigen, an antibody, or a quantum dot). In various embodiments, such particulates and nanoparticles are neutral, or carry a positive charge, or carry a negative charge.

Embodiments of compositions including particulates include those formulated, e.g., as liquids, colloids, dispersions, suspensions, aerosols, gels, and solids. Embodiments include nanoparticles affixed to a surface or support, e.g., an array of carbon nanotubes vertically aligned on a silicon or copper wafer substrate. Embodiments include polynucleotide compositions including particulates (e.g., gold or tungsten or magnetic particles) delivered by a Biolistic-type technique or with magnetic force. The size of the particles used in Biolistics is generally in the “microparticle” range, for example, gold microcarriers in the 0.6, 1.0, and 1.6 micrometer size ranges (see, e.g., instruction manual for the Helios®) Gene Gun System, Bio-Rad, Hercules, CA; Randolph-Anderson et al. (2015) “Submicron gold particles are superior to larger particles for efficient Biolistic® transformation of organelles and some cell types”, Bio-Rad US/EG Bulletin 2015), but successful Biolistics delivery using larger (40-48-WO 2019/144124 PCT/0S2019/014559 nanometer) nanoparticles has been reported in cultured animal cells; see O'Brian and Lummis (2011) BMC Biotechnol., 11:66-71.

Other embodiments of useful particulates are nanoparticles, which are generally in the nanometer (nm) size range or less than 1 micrometer, e.g., with a diameter of less than about 1 nm, less than about 3 nm, less than about 5 nm, less than about 10 nm, less than about 20 nm, less than about 40 nm, less than about 60 nm, less than about 80 nm, and less than about 100 nm. Specific, non-limiting embodiments of nanoparticles commercially available (all from Sigma-Aldrich Corp., St. Louis, MO) include gold nanoparticles with diameters of 5, 10, or 15 nm; silver nanoparticles with particle sizes of 10, 20, 40, 60, or 100 nm; palladium “nanopowder” of less than 25 nm particle size; single-, double-, and multi-walled carbon nanotubes, e.g., with diameters of 0.7-1.1, 1.3-2.3, 0.7-0.9, or 0.7-1.3 nm, or with nano tube bundle dimensions of 2-10 nm by 1-5 micrometers, 6-9 nm by 5 micrometers, 7-15 nm by 0.5-10 micrometers, 7-12 nm by 0.5-10 micrometers, 110-170 nm by 5-9 micrometers, 6-13 nm by 2.5-20 micrometers. Embodiments include polynucleotide compositions including materials such as gold, silicon, cerium, or carbon, e.g., gold or gold-coated nanoparticles, silicon carbide whiskers, carborundum, porous silica nanoparticles, gelatin/silica nanoparticles, nanoceria or cerium oxide nanoparticles (CNPs), carbon nanotubes (CNTs) such as single-, double-, or multi-walled carbon nanotubes and their chemically functionalized versions (e.g., carbon nanotubes functionalized with amide, amino, carboxylic acid, sulfonic acid, or polyethylene glycol moieties), and graphene or graphene oxide or graphene complexes; see, for example, Wong et al. (2016) Nano Lett., 16:1161-1172; Giraldo et al. (2014) Nature Materials, 13:400-409; Shen et al. (2012) Theranostics, 2:283-294; Kim et al. (2011) Bioconjugate Chem., 22:2558-2567; Wang et al. (2010) J. Am. Chem. Soc. Comm., 132:9274-9276; Zhao et al. (2016) Nanoscale Res. Lett., 11:195-203; and Choi et al. (2016) J. Controlled Release, 235:222-235. See also, for example, the various types of particles and nanoparticles, their preparation, and methods for their use, e.g., in delivering polynucleotides and polypeptides to cells, disclosed in US Patent Application Publications 2010/0311168, 2012/0023619, 2012/0244569, 2013/0145488, 2013/0185823, 2014/0096284, 2015/0040268, 2015/0047074, and 2015/0208663, all of which are incorporated herein by reference in their entirety.

In some aspects, the composition includes an excipient, e.g., a delivery vehicle, adjuvant, diluent, surfactant, stabilizer, or tonicity agent or a combination thereof. In some embodiments, the excipient is a crop oil concentrate, a vegetable oil concentrate, a modified vegetable oil, a nitrogen source, a deposition (drift control) and/or retention agent (with or without ammonium sulfate and/or defoamer), a compatibility agent, a buffering agent and/or acidifier, a water conditioning agent, a basic blend, a spreader-sticker and/or extender, an adjuvant plus foliar fertilizer, an antifoam agent, a foam marker, a scent, or a tank cleaner and/or neutralizer. In some embodiments, the excipient is an adjuvant described in the Compendium of Herbicide Adjuvants (Young et al. (2016). Compendium of Herbicide Adjuvants (13th ed.), Purdue University).

Examples of delivery vehicles and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. Further examples of delivery vehicles include, but are not limited to, solid or liquid excipient materials, solvents, stabilizers, slow-release excipients, colorings, and surface-active substances (surfactants). In some instances, the excipient (e.g., delivery vehicle) is a stabilizing vehicle. In embodiments, the stabilizing vehicle includes, e.g., an epoxidized vegetable oil, an antifoaming agent, e.g. silicone oil, a preservative, a viscosity regulator, a binding agent, or a tackifier. In some instances, the stabilizing vehicle is a buffer suitable for the recombinant polynucleotide. In some instances, the composition is microencapsulated in a polymer bead delivery vehicle. In some instances, the stabilizing vehicle protects the recombinant polynucleotide against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

In some instances, the composition provided herein includes an adjuvant. Adjuvants are agents that do not possess the polynucleotide activity, but impart beneficial properties to a formulation. For example, adjuvants are either pre-mixed in the formulation or added to a spray tank to improve mixing or application or to enhance performance. They are used extensively in products designed for foliar applications. Adjuvants can be used to customize the formulation to specific needs and compensate for local conditions. Adjuvants can be designed to perform specific functions, including wetting, spreading, sticking, reducing evaporation, reducing volatilization, buffering, emulsifying, dispersing, reducing spray drift, and reducing foaming. No single adjuvant can perform all these functions, but compatible adjuvants often can be combined to perform multiple functions simultaneously.

Among nonlimiting examples of adjuvants included in the formulation are binders, dispersants and stabilizers, specifically, for example, casein, gelatin, polysaccharides (e.g., starch, gum arabic, cellulose derivatives, alginic acid, etc.), lignin derivatives, bentonite, sugars, synthetic water-soluble polymers (e.g., polyvinyl alcohol, polyvinylpyrrolidone, polyacrylic acid, etc.), PAP (acidic isopropyl phosphate), BHT (2,6-di-t-butyl-4-methylphenol), BHA (a mixture of 2-t-butyl-4-methoxyphenol and 3-t-butyl-4-methoxyphenol), vegetable oils, mineral oils, fatty acids and fatty acid esters.

In embodiments, the compositions provided herein are in a liquid formulation. Liquid formulations are generally mixed with water, but in some instances are used with crop oil, diesel fuel, kerosene or other light oil as an excipient. The amount of active ingredient (e.g., recombinant polynucleotides) often ranges from about 0.5 to about 80 percent by weight.

In embodiments, an emulsifiable concentrate formulation contains a liquid active ingredient, one or more petroleum-based solvents, and an agent that allows the formulation to be mixed with water to form an emulsion. Such concentrates can be used in agricultural, ornamental and turf, forestry, structural, food processing, livestock, and public health pest formulations. In embodiments, these are adaptable to application equipment from small portable sprayers to hydraulic sprayers, low-volume ground sprayers, mist blowers, and low-volume aircraft sprayers. Some active ingredients readily dissolve in a liquid excipient. When mixed with an excipient, they form a solution that does not settle out or separate, e.g., a homogenous solution. In embodiments, formulations of these types include an active ingredient, a carrier and/or an excipient, and one or more other ingredients. Solutions can be used in any type of sprayer, indoors and outdoors.

In some instances, the composition is formulated as an invert emulsion. An invert emulsion is a water-soluble active ingredient dispersed in an oil excipient. Invert emulsions require an emulsifier that allows the active ingredient to be mixed with a large volume of petroleum-based excipient, usually fuel oil. Invert emulsions aid in reducing drift. With other formulations, some spray drift results when water droplets begin to evaporate before reaching target surfaces; as a result the droplets become very small and lightweight. Because oil evaporates more slowly than water, invert emulsion droplets shrink less and more active ingredient reaches the target. Oil further helps to reduce runoff and improve rain resistance. It further serves as a sticker-spreader by improving surface coverage and absorption. Because droplets are relatively large and heavy, it is difficult to get thorough coverage on the undersides of foliage. Invert emulsions are most commonly used along rights-of-way where drift to susceptible non-target areas can be a problem.

A flowable or liquid formulation combines many of the characteristics of emulsifiable concentrates and wettable powders. Manufacturers use these formulations when the active ingredient is a solid that does not dissolve in either water or oil. The active ingredient, impregnated on a substance such as clay, is ground to a very fine powder. The powder is then suspended in a small amount of liquid. The resulting liquid product is quite thick. Flowables and liquids share many of the features of emulsifiable concentrates, and they have similar disadvantages. They require moderate agitation to keep them in suspension and leave visible residues, similar to those of wettable powders.

Flowables/liquids are easy to handle and apply. Because they are liquids, they are subject to spilling and splashing. They contain solid particles, so they contribute to abrasive wear of nozzles and pumps. Flowable and liquid suspensions settle out in their containers. Because flowable and liquid formulations tend to settle, packaging in containers of five gallons or less makes remixing easier.

Aerosol formulations contain one or more active ingredients and a solvent. Most aerosols contain a low percentage of active ingredients. There are two types of aerosol formulations—the ready-to-use type commonly available in pressurized sealed containers and those products used in electrical or gasoline-powered aerosol generators that release the formulation as a smoke or fog.

Ready to use aerosol formulations are usually small, self-contained units that release the formulation when the nozzle valve is triggered. The formulation is driven through a fine opening by an inert gas under pressure, creating fine droplets. These products are used in greenhouses, in small areas inside buildings, or in localized outdoor areas. Commercial models, which hold five to 5 pounds of active ingredient, are usually refillable.

Smoke or fog aerosol formulations are not under pressure. They are used in machines that break the liquid formulation into a fine mist or fog (aerosol) using a rapidly whirling disk or heated surface.

In some embodiments, the composition comprises a liquid excipient. In embodiments, a liquid excipient includes, for example, aromatic or aliphatic hydrocarbons (e.g., xylene, toluene, alkylnaphthalene, phenylxylylethane, kerosene, gas oil, hexane, cyclohexane, etc.), halogenated hydrocarbons (e.g., chlorobenzene, dichloromethane, dichloroethane, trichloroethane, etc.), alcohols (e.g., methanol, ethanol, isopropyl alcohol, butanol, hexanol, benzyl alcohol, ethylene glycol, etc.), ethers (e.g., diethyl ether, ethylene glycol dimethyl ether, diethylene glycol monomethyl ether, diethylene glycol monoethyl ether, propylene glycol monomethyl ether, tetrahydrofuran, dioxane, etc.), esters (e.g., ethyl acetate, butyl acetate, etc.), ketones (e.g., acetone, methyl ethyl ketone, methyl isobutyl ketone, cyclohexanone, etc.), nitriles (e.g., acetonitrile, isobutyronitrile, etc.), sulfoxides (e.g., dimethyl sulfoxide, etc.), amides (e.g., N,N-dimethylformamide, N,N-dimethylacetamide, cyclic imides (e.g. N-methylpyrrolidone) alkylidene carbonates (e.g., propylene carbonate, etc.), vegetable oil (e.g., soybean oil, cottonseed oil, etc.), vegetable essential oils (e.g., orange oil, hyssop oil, lemon oil, etc.), or water.

In some embodiments, the composition comprises a gaseous excipient. Gaseous excipients include, for example, butane gas, floron gas, liquefied petroleum gas (LPG), dimethyl ether, and carbon dioxide gas.

In some embodiments, the compositions are provided as a dry formulation. Dry formulations can be divided into two types: ready-to-use and concentrates that must be mixed with water to be applied as a spray. Most dust formulations are ready to use and contain a low percentage of active ingredients (less than about 10 percent by weight), plus a very fine, dry inert excipient (e.g., talc, chalk, clay, nut hulls, or volcanic ash). The size of individual dust particles varies. A few dust formulations are concentrates and contain a high percentage of active ingredients. In some embodiments, these are mixed with dry inert excipients before applying. In some embodiments, dusts are used dry and can easily drift to non-target sites.

In some instances, the composition is formulated as a powder. In some instances, the composition is formulated as a wettable powder. Wettable powders are dry, finely ground formulations that look like dusts. They usually must be mixed with water for application as a spray. A few products, however, can be applied either as a dust or as a wettable powder—the choice is left to the applicator. Wettable powders have about 1 to about 95 percent active ingredient by weight; in some cases more than about 50 percent. The particles do not dissolve in water. They settle out quickly unless constantly agitated to keep them suspended. They can be used for most pest problems and in most types of spray equipment where agitation is possible. Wettable powders have excellent residual activity. Because of their physical properties, most of the formulation remains on the surface of treated porous materials such as concrete, plaster, and untreated wood. In such cases, only the water penetrates the material.

In some instances, the composition is formulated as a soluble powder. Soluble powder formulations look like wettable powders. However, when mixed with water, soluble powders dissolve readily and form a true solution. After they are mixed thoroughly, no additional agitation is necessary. The amount of active ingredient in soluble powders ranges from about 15 to about 95 percent by weight; in some cases more than about 50 percent. Soluble powders have all the advantages of wettable powders and none of the disadvantages, except the inhalation hazard during mixing.

In some instances, the composition is formulated as a water-dispersible granule. Water-dispersible granules, also known as dry flowables, are like wettable powders, except instead of being dust-like, they are formulated as small, easily measured granules. Water-dispersible granules must be mixed with water to be applied. Once in water, the granules break apart into fine particles similar to wettable powders. The formulation requires constant agitation to keep it suspended in water. The percentage of active ingredient is high, often as much as 90 percent by weight. Water-dispersible granules share many of the same advantages and disadvantages of wettable powders, except they are more easily measured and mixed. Because of low dust, they cause less inhalation hazard to the applicator during handling.

In some embodiments, the composition comprises a solid excipient. Solid excipients include finely-divided powder or granules of clay (e.g. kaolin clay, diatomaceous earth, bentonite, Fubasami clay, acid clay, etc.), synthetic hydrated silicon oxide, talc, ceramics, other inorganic minerals (e.g., sericite, quartz, sulfur, activated carbon, calcium carbonate, hydrated silica, etc.), a substance which can be sublimated and is in the solid form at room temperature (e.g., 2,4,6-triisopropyl-1,3,5-trioxane, naphthalene, p-dichlorobenzene, camphor, adamantan, etc.); wool; silk; cotton; hemp; pulp; synthetic resins (e.g., polyethylene resins such as low-density polyethylene, straight low-density polyethylene and high-density polyethylene; ethylene-vinyl ester copolymers such as ethylene-vinyl acetate copolymers; ethylene-methacrylic acid ester copolymers such as ethylene-methyl methacrylate copolymers and ethylene-ethyl methacrylate copolymers; ethylene-acrylic acid ester copolymers such as ethylene-methyl acrylate copolymers and ethylene-ethyl acrylate copolymers; ethylene-vinylcarboxylic acid copolymers such as ethylene-acrylic acid copolymers; ethylene-tetracyclododecene copolymers; polypropylene resins such as propylene homopolymers and propylene-ethylene copolymers; poly-4-methylpentene-1, polybutene-1, polybutadiene, polystyrene; acrylonitrile-styrene resins; styrene elastomers such as acrylonitrile-butadiene-styrene resins, styrene-conjugated diene block copolymers, and styrene-conjugated diene block copolymer hydrides; fluororesins; acrylic resins such as poly(methyl methacrylate); polyamide resins such as nylon 6 and nylon 66; polyester resins such as polyethylene terephthalate, polyethylene naphthalate, polybutylene terephthalate, and polycyclohexylenedimethylene terephthalate; polycarbonates, polyacetals, polyacrylsulfones, polyarylates, polyacrylates, hydroxybenzoic acid polyesters, polyetherimides, polyester carbonates, polyphenylene ether resins, polyvinyl chloride, polyvinylidene chloride, polyurethane, and porous resins such as foamed polyurethane, foamed polypropylene, or foamed ethylene, etc.), glasses, metals, ceramics, fibers, cloths, knitted fabrics, sheets, papers, yarn, foam, porous substances, and multifilaments.

In some instances, the composition is provided in a microencapsulated formulation (e.g., a nanocapsule). Microencapsulated formulations are mixed with water and sprayed in the same manner as other sprayable formulations. After spraying, the plastic coating breaks down and slowly releases the active ingredient.

In some instances, the composition is provided in a liposome. In some instances, the composition is provided in a vesicle.

In some instances, a composition provided herein includes a surfactant. Surfactants, also called wetting agents and spreaders, physically alter the surface tension of a spray droplet. For a formulation to perform its function properly, a spray droplet must be able to wet the foliage and spread out evenly over a leaf. Surfactants enlarge the area of formulation coverage, thereby increasing exposure to the active agent. Surfactants are particularly important when applying a formulation to waxy or hairy leaves. Without proper wetting and spreading, spray droplets often run off or fail to cover leaf surfaces adequately. Too much surfactant, however, can cause excessive runoff and reduce efficacy.

Surfactants are classified by the way they ionize or split apart into electrically charged atoms or molecules called ions. A surfactant with a negative charge is anionic. One with a positive charge is cationic, and one with no electrical charge is nonionic. Formulation activity in the presence of a nonionic surfactant can be quite different from activity in the presence of a cationic or anionic surfactant. Selecting the wrong surfactant can reduce the efficacy of a product and injure the target plant. Anionic surfactants are most effective when used with contact pesticides (pesticides that control a pest by direct contact rather than being absorbed systemically). Cationic surfactants should never be used as stand-alone surfactants because they usually are phytotoxic.

Nonionic surfactants, often used with systemic pesticides, help sprays penetrate plant cuticles. Nonionic surfactants are compatible with most pesticides, and most EPA-registered pesticides that require a surfactant recommend a nonionic type. Adjuvants include, but are not limited to, stickers, extenders, plant penetrants, compatibility agents, buffers or pH modifiers, drift control additives, defoaming agents, and thickeners.

Among nonlimiting examples of surfactants included in the compositions described herein are alkyl sulfate ester salts, alkyl sulfonates, alkyl aryl sulfonates, alkyl aryl ethers and polyoxyethylenated products thereof, polyethylene glycol ethers, polyvalent alcohol esters and sugar alcohol derivatives. In some embodiments, the surfactant is a nonionic surfactant, a surfactant plus nitrogen source, an organo-silicone surfactant, or a high surfactant oil concentrate.

In formulations and in the use forms prepared from these formulations, the recombinant polynucleotide can, in embodiments, be in a mixture with other active compounds, such as pesticidal agents (e.g., insecticides, sterilants, acaricides, nematicides, molluscicides, or fungicides, attractants, growth-regulating substances, or herbicides). As used herein, the term “pesticidal agent” refers to any substance or mixture of substances intended for preventing, destroying, repelling, or mitigating any pest. A pesticide can be a chemical substance or biological agent used against pests including insects, mollusks, pathogens, weeds, nematodes, and microbes that compete with humans for food, destroy property, spread disease, or are a nuisance. The term “pesticidal agent” further encompasses other bioactive molecules such as antibiotics, antivirals pesticides, antifungals, antihelminthics, nutrients, pollen, sucrose, and/or agents that stun or slow insect movement.

In instances where the recombinant polynucleotide is applied to plants, a mixture with other known compounds, such as herbicides, fertilizers, growth regulators, safeners, semiochemicals, or else with agents for improving plant properties is also possible.

In another aspect, this disclosure is related to a method of producing a modified plant propagule that comprises at least one plant cell comprising a recombinant RNA molecule. The method, in general, includes the steps of: isolating a plant propagule comprising at least one plant cell comprising a recombinant RNA molecule and a partitiviral RNA-dependent RNA polymerase (RDRP) from a mixed population of plant cells comprising both plant cells comprising the recombinant RNA molecule and plant cells lacking the recombinant RNA molecule, wherein the recombinant RNA molecule comprises, in 5′ to 3′ order, a 5′ replication element that is capable of being recognized by the partitiviral RDRP; a cargo RNA sequence; and a 3′ replication element that is capable of being recognized by the partitiviral RDRP. In certain embodiments, the isolated plant propagule comprising at least one plant cell comprising a recombinant RNA molecule will be free or substantially free of plant cells lacking the recombinant RNA. Such isolated plant propagules which are substantially free of plant cells lacking the recombinant RNA can in certain embodiments comprise plant propagules where at least 75%, 80%, 85%, 90%, 95%, 98%, or 99% of the plant cells in the plant propagule contain the recombinant DNA molecule. In embodiments, the mixed population of plant cells comprise a population of protoplasts or a population of cells in callus, an explant, a plant part, or whole plant. In embodiments, the mixed population of plant cells can comprise a population of plant cells where less than 70%, 60%, 50%, 40%, 30%, 20%, 10%, 5%, or 1% of the plant cells in the population contain the recombinant RNA molecule. In some embodiments, the mixed population of plant cells comprise plant cells comprising the partitiviral RDRP and plant cells lacking the partitiviral RDRP. In certain embodiments, the plant cells lacking the partitiviral RDRP will also lack the recombinant RNA. In other embodiments, the mixed population of plant cells comprise plant cells comprising the partitiviral RDRP. In certain embodiments, the plant cells comprising the partitiviral RDRP can further comprise the recombinant RNA. In some embodiments, the cargo RNA sequence comprises RNA that encodes a selectable or scorable marker. In embodiments, the mixed population of plant cells is screened or selected for the presence of the plant cell comprising the recombinant RNA molecule prior to isolating the plant propagule. In such screens, the mixed population of cells or a portion thereof are subjected to an assay for a screenable marker for the presence of the recombinant RNA molecule (e.g., an RNA sequence diagnostic for presence of the recombinant RNA molecule or a polypeptide encoded by the recombinant RNA molecule) and separated from plant cells lacking the recombinant RNA molecule. In some embodiments, the isolation comprises selecting for the plant cell comprising the recombinant RNA molecule prior to isolating the plant propagule. Examples of such selections in instances where the recombinant RNA encodes a selectable marker (e.g., a protein which confers resistance to a selection agent such as an herbicide or antibiotic) can comprise exposing the mixed population of plant cells to a selection agent (e.g., an herbicide or antibiotic) and isolating plant cells which survive exposure to the selection agent. Examples of selectable marker/selection agent combinations include glyphosate-resistant EPSPS enzymes and/or glyphosate oxidases/glyphosate, a bialaphos resistance (bar) or phosphinothricin acyl transferase (pat) enzyme/glufosinate, or a neomycin phosphotransferase (npt)/neomycin or kanamycin. In certain embodiments, the selectable or scorable marker is an RNA aptamer (e.g., a Broccoli aptamer) or a regulatory RNA (e.g., an siRNA, siRNA precursor, miRNA, or miRNA precursor, or a phased siRNA or phased siRNA precursor that downregulates expression of an endogenous gene in the plant, resulting in a detectable phenotype, e.g., bleaching caused by downregulation of a pigment-producing gene). In embodiments, the mixed population is located within a plant or a plant part. In some embodiments, the plant or plant part is screened or selected for presence of the recombinant RNA molecule prior to isolating the plant propagule. In other embodiments, the plant or plant part is screened or selected for systemic presence of the recombinant RNA molecule prior to isolating the plant propagule. In some embodiments, the plant cells, plant, or plant part in the mixed population or that are isolated lack DNA that encodes the recombinant RNA molecule. In embodiments, the plant propagule comprising the recombinant RNA molecule is isolated by detecting the RNA molecule in one or more plant cells comprising the recombinant RNA molecule and separating the one or more plant cells comprising the recombinant RNA molecule from the plant cells lacking the recombinant DNA molecule. In some embodiments, the plant propagule is a mosaic comprising both plant cells comprising the recombinant RNA molecule and plant cells lacking the recombinant RNA molecule. In other embodiments, the modified plant propagule is a mosaic comprising both plant cells comprising the partitiviral RDRP and plant cells lacking the partitiviral RDRP. In certain embodiments, at least 99%, 98%, 95%, 90%, 85%, 80%, 70%, 60%, 50%, 40%, 30%, 20%, 10%, 5%, or 1% of the plant cells in the mosaic can comprise the recombinant RNA molecule. In certain embodiments that are particularly advantageous for at least regulatory reasons, the plant propagule lacks DNA that encodes the recombinant RNA molecule. In embodiments, the modified plant propagule comprises the cell comprising the recombinant RNA molecule, or a seed, seedling, ovule, embryo, pollen, root, stem, leaf, shoot, tuber, rhizome, stolon, bulb, explant, or callus comprising the cell comprising the recombinant RNA molecule. Plant propagules made by any of the aforementioned methods and/or incorporating any of the aforementioned features are also provided herein.

In some embodiments, any of the aforementioned methods can further comprise multiplying the cell, seed, seedling, ovule, embryo, pollen, root, stem, leaf, shoot, tuber, rhizome, stolon, bulb, explant, or callus to obtain progeny, wherein the progeny comprise the recombinant RNA molecule. In other embodiments, the multiplying of the cells consists of culturing a plurality of explants obtained from the cell, seed, seedling, ovule, embryo, pollen, root, stem, leaf, shoot, tuber, rhizome, stolon, bulb, explant, or callus. In yet other embodiments, the isolated propagule comprises the cell and the aforementioned methods can further comprise regenerating a plant, seedling, ovule, embryo, pollen, root, stem, leaf, shoot, tuber, rhizome, stolon, bulb, explant, or callus comprising the recombinant RNA from the cell. In still other embodiments, the isolated propagule comprises callus and the aforementioned methods can further comprise regenerating a plant, seedling, ovule, embryo, pollen, root, stem, leaf, shoot, tuber, rhizome, stolon, bulb, or explant comprising the recombinant RNA from said callus. In embodiments, a plant is regenerated and the aforementioned methods can further comprise recovering F1 seed or F1 progeny or clonal progeny comprising the recombinant RNA from the plant.

In another aspect, this disclosure is related to a method of providing a synthetic partitiviral satellite RNA to a plant or plant part by grafting one plant part to another plant part. In certain embodiments, the methods can comprise grafting a scion onto a rootstock comprising any of the aforementioned or otherwise disclosed recombinant DNA molecules and/or recombinant RNA molecules (e.g., a recombinant RNA comprising, in 5′ to 3′ order, a 5′ replication element that is capable of being recognized by a partitiviral RNA-dependent RNA polymerase (RDRP); a cargo RNA sequence; and a 3′ replication element that is capable of being recognized by the partitiviral RDRP), wherein at least one cell of the rootstock and/or the scion comprises the partitiviral RDRP. In certain embodiments, the scion can comprise a plant shoot, an apical or other meristem, a leaf attached to a petiole, or other plant part and the rootstock can comprise roots and aerial portions of the plant including the main stem, secondary stems, leaves, and/or reproductive structures of the plant, In embodiments, DNA that encodes the recombinant RNA molecule is absent in the scion and/or the rootstock. In embodiments, the scion lacks the recombinant RNA molecule prior to grafting. In embodiments, the rootstock comprises the partitiviral RDRP. In some embodiments, the partitiviral RDRP is provided by a partitivirus endemic to the rootstock (e.g., a partitivirus which is non-pathogenic and/or commensal). In other embodiments, the partitiviral RDRP is exogenously provided to the rootstock (e.g., via DNA expression cassette which is integrated into the chromosomal or plastidic DNA of the rootstock or via a recombinant viral vector comprising DNA or RNA encoding the RDRP). In embodiments, the scion comprises the partitiviral RDRP. In some embodiments, the RDRP is provided by a partitivirus endemic to the scion (e.g., a partitivirus which is non-pathogenic and/or commensal). In other embodiments, the RDRP is exogenously provided to the scion (e.g., via DNA expression cassette which is integrated into the chromosomal or plastidic DNA of the scion or via a recombinant viral vector comprising DNA or RNA encoding the RDRP). In embodiments, the partitiviral RDRP comprises a protein having at least 85%, 90%, 95%, 97%, or 99% sequence identity to SEQ ID NO: 272-290, 384, or 390. In embodiments, the rootstock and/or the scion comprises a heterologous viral coat protein which can encapsidate the recombinant RNA molecule. In some embodiments, the rootstock and/or the scion comprises the recombinant RNA molecule encapsidated by a heterologous viral coat protein.

In another aspect, this disclosure is related to a method of producing a grafted plant comprising a recombinant RNA molecule comprising, in 5′ to 3′ order, a 5′ replication element that is capable of being recognized by a partitiviral RNA-dependent RNA polymerase (RDRP); a cargo RNA sequence; and a 3′ replication element that is capable of being recognized by an the partitiviral RDRP. In certain embodiments, the recombinant RNA molecule is provided by contacting the scion, the rootstock, or both the scion and the rootstock with a composition comprising the recombinant RNA molecule prior to grafting the scion onto the rootstock to produce the grafted plant. In certain embodiments, at least one cell of the rootstock and/or the scion comprises a partitiviral RDRP prior to contacting the scion, the rootstock, or both the scion and the rootstock with the composition. In embodiments, the rootstock comprises the partitiviral RDRP. In some embodiments, the partitiviral RDRP is provided by a partitivirus endemic to the rootstock (e.g., a partitivirus which is non-pathogenic and/or commensal). In other embodiments, the partitiviral RDRP is exogenously provided to the rootstock (e.g., via DNA expression cassette which is integrated into the chromosomal or plastidic DNA of the rootstock or via a recombinant viral vector comprising DNA or RNA encoding the RDRP). In embodiments, the scion comprises the partitiviral RDRP. In some embodiments, the RDRP is provided by a partitivirus endemic to the scion (e.g., a partitivirus which is non-pathogenic and/or commensal). In other embodiments, the RDRP is exogenously provided to the scion (e.g., via DNA expression cassette which is integrated into the chromosomal or plastidic DNA of the scion or via a recombinant viral vector comprising DNA or RNA encoding the RDRP). In embodiments, the partitiviral RDRP comprises a protein having at least 85%, 90%, 95%, 97%, or 99% sequence identity to SEQ ID NO: 272-290, 384, or 390. In embodiments, DNA that encodes the recombinant RNA molecule is absent in the scion, the rootstock, and/or the grafted plant. The composition can be provided to the scion, the rootstock, or both the scion and the rootstock according to any of the formulations disclosed herein. In some embodiments, the formulation is a liquid, a gel, or a powder. In some embodiments, the formulation is configured to be sprayed on to the scion, the rootstock, or both the scion and the rootstock; to be injected into the scion, the rootstock, or both the scion and the rootstock; to be soaked into the scion, the rootstock, or both the scion and the rootstock; or to be coated onto the scion, the rootstock, or both the scion and the rootstock. In certain embodiments, the contacting comprises dipping the scion, the rootstock, or both the scion and the rootstock into the composition prior to grafting.

In another aspect, this disclosure is related to a method for producing a plant that transmits any of the aforementioned or otherwise disclosed recombinant RNA molecules provided herein to progeny plants or seed. In certain embodiments, the methods include the steps of: isolating an F1 progeny plant or seed comprising at least one cell comprising a partitiviral RNA-dependent RNA polymerase (RDRP) and the recombinant RNA molecule comprising, in 5′ to 3′ order, a 5′ replication element that is capable of being recognized by the partitiviral RDRP); a cargo RNA sequence; and a 3′ replication element that is capable of being recognized by the partitiviral RDRP from a population of F₁ plants or seed obtained from at least one parent plant comprising the recombinant RNA molecule. In embodiments, the parent plant or a part thereof comprising the plant cell is screened or selected for presence of the recombinant RNA molecule prior to isolating the F₁ progeny plant or seed. In some embodiments, the parent plant or one or more parts thereof are screened for systemic presence of the recombinant RNA molecule prior to isolating the F₁ progeny plants. In other embodiments, floral tissue (e.g., whole flowers or buds, sepal, calyx, or petal), male reproductive tissue (e.g., stamen, anther, or pollen), or female reproductive tissue (e.g., whole fruit, ovary, pericarp, ovule, seed coat, endosperm, or embryo) of the parent plant is screened or selected for presence of the recombinant RNA molecule. In embodiments, F1 seeds are obtained from a parent plant selected for presence of the recombinant RNA molecule in pericarp tissue. In embodiments, the F₁ progeny plant or seed comprising the cell is isolated by screening the population of F₁ plants or seed obtained from a parent plant for the presence of the recombinant RNA molecule and propagating the F₁ progeny plant or seed comprising the recombinant RNA molecule. In such screens, the progeny plants or seed thereof are subjected to an assay for a screenable marker for the presence of the recombinant RNA (e.g., an RNA sequence diagnostic for presence of the recombinant RNA molecule or a polypeptide encoded by the recombinant RNA molecule) and separated from progeny plants and seed lacking the recombinant RNA progeny plants and seed lacking the recombinant RNA. Such screening assays can be non-destructive assays wherein a portion of the progeny seed or plant is removed and assayed without loss of the viability or ability to propagate the seed or plant tissue comprising the recombinant RNA. In some embodiments, an F₁ seed of the parent plant is non-destructively screened for presence of the recombinant RNA molecule. In other embodiments, the F₁ seed of the parent plant is non-destructively screened by assaying maternally derived or endosperm tissue of the seed for the presence of the recombinant RNA molecule. Methods for non-destructive assays of seed or other plant tissue which can be adapted for such screens include but are not limited to those disclosed in US patent applications US20220221377 and US20210259176, both incorporated herein by reference in their entireties. In some embodiments, the cargo RNA sequence comprises RNA that encodes a selectable or scorable marker. In some embodiments, the recombinant RNA molecule encodes a selectable marker and the F₁ progeny plant or seed comprising the recombinant RNA molecule is isolated by selecting the F₁ progeny plant or seed comprising the recombinant RNA molecule for presence of the selectable marker. Examples of such selections in instances where the recombinant RNA encodes a selectable marker (e.g., a protein which confers resistance to a selection agent such as an herbicide or antibiotic) can comprise exposing the progeny seeds or plants to a selection agent (e.g., an herbicide or antibiotic) and isolating progeny seeds or plants which survive exposure to the selection agent. Examples of selectable marker/selection agent combinations include glyphosate-resistant EPSPS enzymes and/or glyphosate oxidases/glyphosate, a bialaphos resistance (bar) or phosphinothricin acyl transferase (pat) enzymes/glufosinate, and neomycin phosphotransferase (npt)/neomycin or kanamycin. In certain embodiments, the selectable or scorable marker is an RNA aptamer or a regulatory RNA. In some embodiments, the F₁ progeny plant or seed lack DNA that encodes the recombinant RNA molecule. In other embodiments, the parent plant lacks DNA that encodes the recombinant RNA molecule. In embodiments, the selected F1 progeny plant transmits the recombinant RNA molecule to at least F2 progeny. In some embodiments, the F1 progeny plant or seed population is obtained from a parent plant used as a pollen recipient. In other embodiments, the F1 progeny plant or seed population is obtained from a parent plant used as a pollen donor. In embodiments, the F1 progeny plant or seed population is obtained by selfing the parent plant. In other embodiments, the F₁ progeny plant or seed population is obtained from the sexual crossing of two parent plants. In some embodiments, the parent plant that comprises the recombinant RNA molecule is the female parent plant. In other embodiments, the parent plant that comprises the recombinant RNA molecule is the male parent plant, and the recombinant RNA molecule is transmitted in pollen of the male parent plant.

In certain embodiments, the methods can further comprise introducing the recombinant RNA molecule or a polynucleotide encoding the recombinant RNA molecule into a plant cell and obtaining the parent plant comprising the recombinant RNA molecule from the plant cell. In embodiments, the recombinant RNA molecule further comprises at least one additional element selected from the group consisting of: (a) at least one RNA encoding a viral movement protein (MP); (b) at least one tRNA-like sequence; and c) an origin-of-assembly sequence (OAS). In embodiments, a parent and/or plant comprises a heterologous viral coat protein which can encapsidate the recombinant RNA molecule. In some embodiments, the parent and/or progeny plant comprises the recombinant RNA molecule encapsidated by a heterologous viral coat protein.

In another aspect, this disclosure is related to a method of barcoding a plant, plant cell, progeny thereof, or part thereof. The methods comprise providing to the plant or plant cell any of the aforementioned or otherwise disclosed recombinant RNA molecules provided herein, wherein the cargo RNA of the recombinant RNA molecule comprises a barcode RNA molecule, and wherein the plant or plant cell comprises a partitiviral RDRP. In embodiments, the barcode RNA molecule comprises a sequence that uniquely identifies the plant, plant cell, progeny thereof, or part thereof. In some embodiments, the barcode RNA can be a randomly generated sequence. In some embodiments, the barcode RNA molecule comprises a sequence that is not present in the genome and/or transcriptome of a wild-type plant of the same species, a pathogen thereof, or a symbiont thereof. In some embodiments, the barcode RNA molecule comprises a forward primer binding site and a reverse primer binding site for detection of the barcode RNA molecule. In embodiments, the barcode RNA molecule comprises a non-protein coding sequence. In some embodiments, the barcode RNA sequence is up to about 3.2 kb in length. In some embodiments, the barcode RNA has a length of 10 to 5000 nucleotides, 20 to 1000 nucleotides, or 50 to 500 nucleotides. In certain embodiments, the barcode RNA molecule has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, or 5000 nucleotides. In embodiments, the plant transmits the recombinant RNA molecule comprising the barcode RNA to progeny. In some embodiments, the plant, plant cell, progeny thereof, or part thereof lacks DNA that encodes the recombinant RNA molecule. In some embodiments, the methods can further comprise isolating an F₁ progeny plant or seed comprising at least one cell comprising the partitiviral RDRP and the recombinant RNA molecule. In some embodiments, the F₁ progeny plant or seed is obtained from the plant used as a pollen recipient. In other embodiments, the F₁ progeny plant or seed is obtained from the plant used as a pollen donor. In embodiments, the F₁ progeny plant or seed is obtained by selfing the parent plant. In embodiments, the methods can further comprise propagating the plant or plant cell to obtain a plant part or a plant propagule comprising the barcode RNA molecule.

In another aspect, this disclosure is related to a method of identifying a barcoded plant, plant part, or plant cell. The methods comprise screening for the presence of a barcode RNA molecule in the plant, plant part, or plant cell, wherein the plant, plant part, or plant cell comprises any of the aforementioned or otherwise disclosed recombinant RNA molecules provided herein, and wherein the cargo RNA of the recombinant RNA molecule comprises the barcode RNA molecule. In certain embodiments, the methods comprise obtaining a nucleic acid sample from the plant, plant part, or plant cell; and detecting the presence of the barcode RNA molecule in the sample. Assays for detection of a barcode RNA include RNA detection assays (e.g., an RT-PCR assay) using nucleic acid probes and/or primers which can detect the barcode RNA and/or sequencing of the barcode RNA. Such screening assays can be non-destructive assays wherein a portion of the seed or plant is removed and assayed without loss of the viability or ability to propagate the seed or plant tissue comprising the barcode RNA. Methods for non-destructive assays of seed or other plant tissue which can be adapted for such screens include but are not limited to those disclosed in US patent applications US20220221377 and US20210259176, both incorporated herein by reference in their entireties. In some embodiments, a seed of the plant is non-destructively screened for presence of the barcode RNA molecule. In other embodiments, a seedling, ovule, embryo, pollen, root, stem, leaf, shoot, tuber, rhizome, stolon, bulb, explant, or callus is screened for the presence of the barcode RNA molecule.

In certain optional embodiments, the methods disclosed herein are not processes for modifying the germ line genetic identity of human beings. In certain optional embodiments, the methods disclosed herein are not processes for modifying the genetic identity of animals which are likely to cause them suffering without any substantial medical benefit to man or animal, and are also not drawn to animals resulting from such processes. In certain optional embodiments, the methods disclosed herein are not methods for treatment of the human or animal body by surgery or therapy. In a certain optional embodiments, the cells disclosed herein are not human embryos. In certain optional embodiments, the cells disclosed herein are not the human body, at the various stages of its formation and development. In certain optional embodiments provided herein, the plant cells, plant propagules (e.g., a seed, seedling, ovule, embryo, pollen, root, stem, leaf, shoot, tuber, rhizome, stolon, bulb, explant, or callus), and plants provided herein are not produced by an exclusively biological process. In certain optional embodiments provided herein, the methods for producing plant cells, plant propagules (e.g., a seed, seedling, ovule, embryo, pollen, root, stem, leaf, shoot, tuber, rhizome, stolon, bulb, explant, or callus), and plants provided herein are not exclusively biological processes.

### Embodiments

Various embodiments of the compositions, systems, and methods described herein are set forth in the following set of numbered embodiments.

1. A recombinant DNA molecule comprising a first promoter which is operably linked to DNA encoding an RNA molecule, wherein the RNA molecule comprises from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP; wherein the 5′ RNA replication element, the cargo RNA molecule, and the 3′ RNA replication element are operably linked, wherein the promoter and the cargo RNA molecule are heterologous to the 5′ RNA replication element and the 3′ RNA replication element.

2. The recombinant DNA molecule of embodiment 1, wherein: (a) the 5′ RNA replication element comprises at least one RNA secondary structure provided in Table 1 or adopted by an RNA encoded by SEQ ID NO: 1-34, 69-88, 372, 374, 376, 378, 380, 382, or 386; and/or (b) the 3′ RNA replication element comprises at least one RNA secondary structure provided in Table 1 or adopted by an RNA encoded by SEQ ID NO: 35-68, 89-108, 373, 375, 377, 379, 381, 383, or 387.

3. The recombinant DNA molecule of embodiment 1 or 2, wherein: (a) the 5′ RNA replication element is encoded by a DNA molecule comprising SEQ ID NO: 1-34, 69-88, 372, 374, 376, 378, 380, 382, or 386; a variant thereof comprising DNA having at least 70%, 75%, 80%, 85%, 90%, 95%, 98%, or 99% sequence identity to SEQ ID NO: 1-34, 69-88, 372, 374, 376, 378, 380, 382, or 386; or a variant thereof wherein one or more nucleotides in the RNA secondary structure are substituted with distinct nucleotides which maintain the RNA secondary structure; and/or (b) the 3′ RNA replication element is encoded by a DNA molecule comprising SEQ ID NO: 35-68, 89-108, 373, 375, 377, 379, 381, 383, or 387; a variant thereof comprising DNA having at least 70%, 75%, 80%, 85%, 90%, 95%, 98%, or 99% sequence identity to SEQ ID NO: 35-68, 89-108, 373, 375, 377, 379, 381, 383, or 387; or a variant thereof wherein one of more residues in the RNA secondary structure are substituted with distinct nucleotides which maintain the RNA secondary structure.

4. The recombinant DNA molecule of embodiment 1, 2, or 3, wherein the RNA secondary structure is maintained by substituting nucleotides in the secondary structure which are not base paired with nucleotides which will not base pair and/or by substituting nucleotides in the secondary structure which are base paired with nucleotides which will base pair.

5. The recombinant DNA molecule of any one of embodiments 1 to 4, wherein: (a) the 5′ RNA replication element comprises an RNA molecule containing at least a segment of the 5′ untranslated region (UTR) of the partitiviral genome or a variant thereof having one or more nucleotide substitutions, insertions, and/or deletions, wherein the variant is recognized by the RDRP, optionally wherein the 5′ replicase replication element further comprises a genomic sequence of the partitivirus that is natively located 3′ to and adjacent to the 5′ UTR sequence; and/or (b) the 3′ RNA replication element comprises an RNA molecule containing at least a segment of the 3′ UTR of the partitiviral genome or a variant thereof having one or more nucleotide substitutions, insertions, and/or deletions, wherein the variant is recognized by the RDRP, optionally wherein the 3′ replicase replication element further comprises a genomic sequence of the partitivirus that is natively located 5′ to and adjacent to the 3′ UTR sequence.

6. The recombinant DNA of any one of embodiments 1 to 5, wherein the 5′ RNA replication element and the 3′ RNA replication element are obtained from the same partitiviral genome or from related partitiviral genomes having at least 85%, 90%, 95%, 98%, or 99% sequence identity to one another, optionally wherein the 5′ RNA replication element, the 3′ RNA replication element, and the RDRP are obtained from the same partitiviral genome or from related partitiviral genomes having at least 85%, 90%, 95%, 98%, or 99% sequence identity to one another, or optionally wherein the 5′ RNA replication element, the 3′ RNA replication element, and/or the RDRP coding region are obtained from two partitiviral genomes wherein the members of each pair of the 5′ RNA replication elements, 3′ RNA replication elements, and/or RDRP coding regions of the two partitiviral genomes have at least 85%, 90%, 95%, 98%, or 99% sequence identity to one another.

7. The recombinant DNA of embodiment 6, wherein the 5′ RNA replication element of (a) and the 3′ RNA replication element (b) are obtained from distinct partitiviral genomes, optionally wherein the distinct partitiviral genomes have less than 85%, 80%, 75%, or 70% sequence identity to one another or optionally wherein the distinct partitiviral genomes have 50%, 60%, or 65% to any one of 70%, 75%, 80%, or 84% sequence identity to one another.

8. The recombinant DNA molecule of any one of embodiments 1 to 7, wherein the RNA molecule further comprises at least one of: (i) a tRNA-like element, optionally wherein the tRNA-like element provides for intercellular movement of the RNA and optionally wherein the intercellular movement is mediated by a viral movement protein (MP); (ii) an encapsidation recognition element (ERE), optionally wherein the ERE provides for encapsidation of the RNA by a partitiviral capsid protein or optionally wherein the ERE provides for encapsidation of the RNA by a non-partitiviral capsid protein; (iii) a RNA effecter; and/or (iv) RNA encoding a viral movement protein (MP), optionally wherein an internal ribosome entry site (IRES) is operably linked to the RNA encoding the MP.

9. The recombinant DNA molecule of embodiment 8, wherein the encapsidation recognition element (ERE) comprises a viral origin-of-assembly sequence (OAS), optionally wherein the OAS is a Tobamovirus OAS.

10. The recombinant DNA molecule of embodiment 8, wherein the tRNA-like element comprises a tRNA-like molecule from an Arabidopsis FT mRNA and/or the ERE is a tobacco mosaic virus (TMV) OAS.

11. The recombinant DNA molecule of any one of embodiments 1 to 10, wherein the DNA molecule further comprises a second promoter which is operably linked to DNA encoding an RNA molecule comprising from 5′ terminus to 3′ terminus: (i) a 5′ RNA replication element recognized by a partitiviral RNA-dependent RNA polymerase (RDRP); (ii) RNA encoding a viral movement protein (MP), optionally wherein an internal ribosome entry site (IRES) is operably linked to the RNA; and (iii) a 3′ RNA replication element recognized by the RDRP, optionally wherein the RNA further comprises at least one of: (a) a tRNA-like element, optionally wherein the tRNA-like element provides for intercellular movement of the RNA and optionally wherein the intercellular movement is mediated by a viral movement protein (MP); (b) an RNA effecter; and/or (c) an encapsidation recognition element (ERE), optionally wherein the ERE provides for encapsidation of the RNA by a partitiviral capsid protein or optionally wherein the ERE provides for encapsidation of the RNA by a non-partitiviral capsid protein.

12. The recombinant DNA molecule of embodiment 11, wherein the RNA encoding a viral MP is heterologous to the 5′ RNA replication element and the 3′ RNA replication element which are operably linked to the second promoter.

13. The recombinant DNA molecule of embodiment 8 or 11, wherein the RNA effecter provides for RNA cleavage, protein binding, or RNA synthesis, optionally wherein the RNA effecter comprises a ribozyme, a ligand-responsive ribozyme (aptazyme), an RNA aptamer, or an RNA promoter recognized by an RNA promoter dependent RNA polymerase (RPDRP), optionally wherein the operably linked to the RNA molecule, and/or optionally wherein the RNA effecter is operably linked to the 5′ RNA replication element, the cargo RNA molecule; and/or to the 3′ RNA replication element.

14. The recombinant DNA molecule of any one of embodiments 1 to 13, wherein the DNA molecule further comprises: (i) a promoter which is operably linked to DNA encoding the partitiviral RNA-dependent RNA polymerase (RDRP) and an operably linked terminator element; and/or (ii) a promoter which is operably linked to DNA encoding a capsid protein that recognizes the ERE and an operably linked terminator element, optionally wherein the capsid protein is a partitiviral capsid protein.

15. The recombinant DNA molecule of any one of embodiments 1 to 14, wherein the cargo RNA molecule is up to about 3.2 kBp in length.

16. The recombinant DNA molecule of any one of embodiments 1 to 15, wherein the cargo RNA molecule comprises: (a) at least one coding sequence, optionally wherein the cargo sequence encodes a selectable or scorable marker; (b) at least one non-coding sequence; or (c) both at least one coding sequence and at least one non-coding sequence.

17. The recombinant DNA molecule of any one of embodiments 1 to 16, wherein the cargo RNA molecule comprises at least one coding sequence, and wherein the RNA molecule further comprises an internal ribosome entry site (IRES) which is operably linked to at least one coding sequence, optionally wherein the operably linked IRES is located 5′ and immediately adjacent to the coding sequence.

18. The recombinant DNA molecule of any one of embodiments 1 to 17, wherein the cargo RNA molecule comprises multiple coding sequences, and wherein the RNA molecule further comprises an IRES which is operably linked to each of the coding sequences, optionally wherein the operably linked IRES is located 5′ and immediately adjacent to a coding sequence in the cargo RNA molecule.

19. The recombinant DNA molecule of any one of embodiments 1 to 17, wherein the cargo RNA molecule comprises at least one non-coding sequence, and wherein the at least one non-coding sequence is a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ribozyme cleavage site; a ligand-responsive ribozyme (aptazyme); an RNA aptamer; or a long noncoding RNA (lncRNA).

20. The recombinant DNA molecule of any one of embodiments 1 to 19, further comprising a DNA molecule encoding at least one ribozyme, optionally wherein the at least one ribozyme is located 5′ to the 5′ RNA replication element or 3′ to the 3′ RNA replication element.

21. The recombinant DNA molecule of any one of embodiments 1 to 20, further comprising a DNA molecule encoding at least one ligand-responsive ribozyme (aptazyme), optionally wherein the at least one ligand-responsive ribozyme is located 5′ to the 5′ RNA replication element or 3′ to the 3′ RNA replication element.

22. The recombinant DNA molecule of any one of embodiments 1 to 20, further comprising DNA encoding an intron, optionally wherein the DNA encoding the intron is located between: (i) the 3′ end of the promoter and the 5′ end of the DNA encoding the 5′ RNA replication element; (ii) the 3′end of the 5′ RNA replication element and the 5′ end of the DNA encoding the cargo RNA molecule; or (iii) the 3′ end of the DNA encoding the cargo RNA molecule and the 5′ end of the 3′ RNA replication element.

23. The recombinant DNA molecule of any one of embodiments 1 to 22, further comprising at least one additional element comprising: a discrete expression cassette comprising a second promoter operably linked to a DNA molecule to be transcribed, and optionally a terminator element; an expression-enhancing element operably linked to a promoter or RNA encoding sequence, wherein the expression enhancing element is optionally a transcription enhancer, a translation enhancer encoding sequence, or an intron encoding sequence; a DNA molecule encoding a selectable or scoreable marker; a DNA aptamer; a DNA molecule encoding an RNA aptamer; an Agrobacterium T-DNA left and right border DNA element operably linked to the 5′ and 3′ end of the recombinant DNA molecule; spacer DNA molecule or DNA encoding an RNA cleavage agent recognition site, optionally wherein the RNA cleavage agent comprises a guide RNA and Cas nuclease, an siRNA, ta-siRNA, a ribozyme, aptazyme, or an miRNA; a DNA molecule encoding a transcription factor binding site operably linked to a promoter; a DNA molecule encoding a localization sequence which is operably linked to one or more coding sequences, optionally wherein the coding sequence comprises the cargo RNA; a DNA molecule encoding at least one sequence-specific recombinase recognition site (SSRRS), optionally wherein the SSRRS is operably linked to one or more of: (i) the DNA encoding the cargo RNA; (ii) a coding sequence of the DNA molecule; (iii) the 5′ and/or 3′ end of the DNA molecule; and/or the 5′ and/or 3′ end of the segment of the DNA molecule encoding the RNA molecule; and/or a DNA molecule encoding a transcript-stabilizing or transcript-destabilizing RNA sequence, wherein the transcript-stabilizing or transcript-destabilizing RNA sequence is operably linked to the RNA molecule encoded by the DNA molecule.

24. The RNA molecule encoded by the recombinant DNA molecule of any one of embodiments 1 to 23.

25. A cell comprising the recombinant DNA molecule of any one of embodiments 1 to 23, optionally wherein the cell is a prokaryotic cell or a eukaryotic cell, and optionally wherein the eukaryotic cell is a plant cell.

26. A vector for bacterially-mediated plant transformation, comprising the recombinant DNA molecule of any one of embodiments 1 to 23.

27. The vector of embodiment 26, wherein the bacterium that mediates the plant transformation is an Agrobacterium sp., a Sinorhizobium sp., a Mesorhizobium sp., a Bradyrhizobium sp., Rhizobium sp., or an Ensifer sp. and the vector is adapted for transformation with the bacterium.

28. The vector of embodiment 26 or 27, wherein the bacterium that mediates the plant transformation is an Agrobacterium sp., and wherein the vector further comprises T-DNAs flanking the DNA molecule encoding the recombinant RNA molecule.

29. The vector of embodiment 26, 27 or 28, contained within a bacterial or plant cell.

30. An expression system comprising: (a) the recombinant DNA molecule of any one of embodiments 1 to 23; and (b) a cell containing the recombinant DNA molecule and an RDRP protein that recognizes the 5′ and 3′ RNA replication elements encoded by the DNA molecule.

31. The expression system of embodiment 30, wherein the recombinant DNA molecule further comprises at least one additional element comprising: (i) DNA encoding at least one RNA encoding a viral movement protein (MP); (ii) DNA encoding at least one tRNA-like molecule; (iii) DNA encoding an encapsidation recognition element (ERE); (iv) DNA encoding an RNA comprising, from 5′ to 3′ and operably linked, a 5′ RNA replication element recognized by a partitiviral RNA-dependent RNA polymerase (RDRP), the RNA of (i) and optionally an operably linked RNA of (ii) and/or (iii), and a 3′ RNA replication element; (v) DNA encoding an RNA promoter; (vi) DNA encoding an RNA promoter dependent RNA polymerase (RPDRP); and/or (vii) a DNA molecule comprising a promoter which is operably linked to the DNA encoding the RNA of at least one of (i), (ii), (iii), (iv), (v), or (vi).

32. The expression system of embodiment 30 or 31, wherein the expression system further comprises a DNA molecule comprising: (i) DNA encoding at least one RNA encoding a viral movement protein (MP); (ii) DNA encoding at least one tRNA-like molecule; (iii) DNA encoding an encapsidation recognition element (ERE); (iv) DNA encoding an RNA comprising, from 5′ to 3′ and operably linked, a 5′ RNA replication element recognized by a partitiviral RNA-dependent RNA polymerase (RDRP), the RNA of (i) and optionally an operably linked RNA of (ii) and/or (iii), and a 3′ RNA replication element; (v) DNA encoding an RNA promoter; (vi) DNA encoding an RNA promoter dependent RNA polymerase (RPDRP); and/or (vii) a DNA molecule comprising a promoter which is operably linked to the DNA encoding the RNA of at least one of (i), (ii), (iii), (iv), (v), or (vi).

33. The expression system of any one of embodiments 30 to 32, wherein the cell is a bacterial cell, a plant cell, a fungal cell, or an animal cell, optionally wherein the bacterial cell is an Agrobacterium sp., a Sinorhizobium sp., a Mesorhizobium sp., Bradyrhizobium sp., Rhizobium sp., or an Ensifer sp. cell.

34. The expression system of any one of embodiments 30 to 33, further comprising: (i) a viral capsid protein that can encapsidate an RNA molecule comprising the encapsidation recognition element (ERE); (ii) an RNA-binding protein (RBP) that can bind to the RNA molecule encoded by the DNA molecule, optionally wherein the RBP binds to an RNA effecter; (iii) an RNA cleavage agent that cleaves the RNA molecule; and/or (iv) an RNA promoter dependent RNA polymerase (RPDRP) protein that recognizes an RNA promoter in the RNA molecule.

35. The expression system of embodiment 34, wherein the viral capsid protein is: (a) expressed by the recombinant DNA molecule in the cell, (b) co-expressed by a second recombinant DNA molecule in the cell; (c) provided exogenously to the cell; or (d) expressed by a virus in the cell.

36. The expression system of any one of embodiments 30 to 35, wherein: (i) the capsid protein, viral movement protein (MP), RDRP protein, and/or the RPDRP protein is heterologous to the cell and/or (ii) wherein the RDRP protein or a polynucleotide encoding the RDRP protein is provided exogenously to the cell.

37. The expression system of any one of embodiments 30 to 36, wherein the cell is a plant cell.

38. The expression system of embodiment 37, wherein the plant cell contains a partitivirus which expresses the RDRP protein that recognizes the 5′ RNA replication element and the 3′ RNA replication element.

39. The expression system of embodiment 38, wherein the partitivirus occurs naturally in the plant cell.

40. The expression system of any one of embodiments 30 to 39, wherein the recombinant DNA molecule further comprises at least one RNA encoding a viral MP, a tRNA-like molecule from an Arabidopsis FT mRNA, and an encapsidation recognition element comprising a TMV-OAS.

41. An agricultural formulation comprising the expression system of any one of embodiments 30 to 40.

42. The agricultural formulation of embodiment 41, wherein the formulation comprises the expression system and a carrier, an excipient and/or an adjuvant.

43. An agricultural formulation comprising the recombinant DNA molecule of any one of embodiments 1 to 23.

44. The agricultural formulation of embodiment 43, wherein the formulation comprises the recombinant DNA molecule and a carrier, an excipient and/or an adjuvant.

45. A method of providing a synthetic partitiviral satellite RNA to a plant, comprising contacting the plant with the recombinant DNA molecule of any one of embodiments 1 to 23, the RNA of embodiment 24, the cell of embodiment 25, the vector of any one of embodiments 26 to 29, or the formulation of embodiment 43 or 44.

46. The method of embodiment 45, wherein contacting comprises spraying, dusting, injecting, or soaking with the recombinant DNA molecule, the vector, or the formulation.

47. A recombinant RNA molecule comprising from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP, wherein the 5′ RNA replication element, the cargo RNA molecule, and the 3′ RNA replication element are operably linked and wherein the cargo RNA molecule is heterologous to the 5′ RNA replication element and the 3′ RNA replication element, optionally wherein: (i) the 5′ RNA replication element and the 3′ RNA replication element are obtained from the same partitiviral genome or from related partitiviral genomes having at least 85%, 90%, 95%, 98%, or 99% sequence identity to one another; (ii) the 5′ RNA replication element, the 3′ RNA replication element, and the RDRP are obtained from the same partitiviral genome or from related partitiviral genomes having at least 85%, 90%, 95%, 98%, or 99% sequence identity to one another; or (iii) the 5′ RNA replication element, the 3′ RNA replication element, and/or the RDRP coding region are obtained from two partitiviral genomes wherein the members of each pair of the 5′ RNA replication elements, 3′ RNA replication elements, and/or RDRP coding regions of the two partitiviral genomes have at least 85%, 90%, 95%, 98%, or 99% sequence identity to one another.

48. The recombinant RNA molecule of embodiment 47, wherein: (a) the 5′ RNA replication element comprises at least one RNA secondary structure provided in Table 1 or adopted by an RNA molecule encoded by SEQ ID NO: 1-34, 69-88, 372, 374, 376, 378, 380, 382, or 386; and/or (b) the 3′ RNA replication element comprises at least one RNA secondary structure provided in Table 1 or adopted by an RNA molecule encoded by SEQ ID NO: 35-68, 89-108, 373, 375, 377, 379, 381, 383, or 387.

49. The recombinant RNA molecule of embodiment 47 or 48, wherein: (a) the 5′ RNA replication element comprises an RNA molecule encoded by SEQ ID NO: 1-34, 69-88, 372, 374, 376, 378, 380, 382, or 386; a variant thereof encoded by a DNA molecule having at least 70%, 75%, 80%, 85%, 90%, 95%, 98%, or 99% sequence identity to SEQ ID NO: 1-34, 69-88, 372, 374, 376, 378, 380, 382, or 386; or a variant thereof wherein one or more nucleotides in the RNA secondary structure are substituted with distinct nucleotides which maintain the RNA secondary structure; and/or (b) the 3′ RNA replication element comprises an RNA encoded by SEQ ID NO: 35-68, 89-108, 373, 375, 377, 379, 381, 383, or 387, a variant thereof encoded by a DNA molecule having at least 70%, 75%, 80%, 85%, 90%, 95%, 98%, or 99% sequence identity to SEQ ID NO: 35-68, 89-108, 373, 375, 377, 379, 381, 383, or 387 and/or a variant thereof wherein one of more base-paired residues in the RNA secondary structure are substituted with distinct nucleotides which maintain the RNA secondary structure.

50. The recombinant RNA molecule of embodiment 49, wherein the RNA secondary structure is maintained by substituting nucleotides in the secondary structure which are not base paired with nucleotides which will not base pair and/or by substituting nucleotides in the secondary structure which are base paired with nucleotides which will base pair.

51. The recombinant RNA molecule of any one of embodiments 47 to 50, wherein: (a) the 5′ RNA replication element comprises at least a segment of the 5′ untranslated region (UTR) of the partitiviral genome or a variant thereof having one or more nucleotide substitutions, insertions, and/or deletions, wherein the variant is recognized by the RDRP, optionally wherein the 5′ replicase replication element further comprises a genomic sequence of the partitivirus that is natively located 3′ to and adjacent to the 5′ UTR sequence; and/or (b) the 3′ RNA replication element comprises at least a segment of the 3′ UTR of the partitiviral genome or a variant thereof having one or more nucleotide substitutions, insertions, and/or deletions, wherein the variant is recognized by the RDRP, optionally wherein the 3′ replicase replication element further comprises a genomic sequence of the partitivirus that is natively located 5′ to and adjacent to the 3′ UTR sequence, and optionally wherein the partitiviral genome of (a) and (b) are the same.

52. The recombinant RNA molecule of embodiments 47 to 51, wherein the RNA molecule further comprises at least one of: (i) a tRNA-like element, optionally wherein the tRNA-like element provides for intercellular movement of the RNA and optionally wherein the intercellular movement is mediated by a viral movement protein (MP); (ii) an encapsidation recognition element (ERE), optionally wherein the ERE provides for encapsidation of the RNA by a partitiviral capsid protein or optionally wherein the ERE provides for encapsidation of the RNA by a non-partitiviral capsid protein; (iii) an RNA effecter; and/or (iv) RNA encoding a viral movement protein (MP), optionally wherein an internal ribosome entry site (IRES) is operably linked to the RNA encoding the MP.

53. The recombinant RNA molecule of embodiment 52, wherein the RNA effecter provides for RNA cleavage, protein binding, or RNA synthesis, optionally wherein the RNA effecter comprises a ribozyme, a ligand-responsive ribozyme (aptazyme), an RNA aptamer, or an RNA promoter recognized by an RNA promoter dependent RNA polymerase (RPDRP), optionally wherein the operably linked to the RNA molecule and/or optionally wherein the RNA effecter is operably linked to the 5′ RNA replication element, the cargo RNA molecule; and/or to the 3′ RNA replication element.

54. The recombinant RNA molecule of embodiment 52, wherein the encapsidation recognition element (ERE) comprises a viral origin-of-assembly sequence (OAS), optionally wherein the OAS is a Tobamovirus OAS.

55. The recombinant RNA molecule of embodiment 52, wherein the tRNA-like element comprises a tRNA-like molecule from an Arabidopsis FT mRNA and/or the ERE is a tobacco mosaic virus (TMV) OAS.

56. The recombinant RNA molecule of any one of embodiments 47 to 55, wherein the cargo RNA molecule is up to about 3.2 kb in length.

57. The recombinant RNA molecule of any one of embodiments 47 to 56, wherein the cargo RNA molecule comprises: (a) at least one coding sequence, optionally wherein the coding sequence encodes a selectable or scoreable marker; (b) at least one non-coding sequence; or (c) both at least one coding sequence and at least one non-coding sequence.

58. The recombinant RNA molecule of any one of embodiments 47 to 57, wherein the cargo RNA molecule comprises at least one coding sequence, and wherein the RNA molecule further comprises an internal ribosome entry site (IRES) which is operably linked to at least one coding sequence, optionally wherein the operably linked IRES is located 5′ and immediately adjacent to the coding sequence.

59. The recombinant RNA molecule of any one of embodiments 47 to 58, wherein the cargo RNA molecule comprises multiple coding sequences, and wherein the RNA molecule further comprises an IRES which is operably linked to each of the coding sequences, optionally wherein the operably linked IRES is located 5′ and immediately adjacent to a coding sequence. In the cargo RNA molecule.

60. The recombinant RNA molecule of any one of embodiments 47 to 57, wherein the cargo RNA molecule comprises at least one non-coding sequence, and wherein the at least one non-coding sequence is a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ligand-responsive ribozyme (aptazyme); an RNA aptamer; or a long noncoding RNA (lncRNA).

61. The recombinant RNA molecule of any one of embodiments 47 to 60, further comprising an RNA comprising encoding at least one ribozyme, optionally wherein the at least one ribozyme is located 5′ to the 5′ RNA replication element or 3′ to the 3′ RNA replication element.

62. The recombinant RNA molecule of any one of embodiments 47 to 61, further comprising an RNA molecule comprising at least one ligand-responsive ribozyme (aptazyme), optionally wherein the at least one ligand-responsive ribozyme is located 5′ to the 5′ RNA replication element or 3′ to the 3′ RNA replication element.

63. The recombinant RNA molecule of any one of embodiments 47 to 62, further comprising RNA encoding an intron, optionally wherein the RNA encoding the intron is located between the 3′end of the 5′ RNA replication element and the RNA encoding the cargo RNA.

64. The recombinant RNA molecule of any one of embodiments 47 to 63, further comprising at least one additional element comprising: an RNA aptamer; an RNA molecule encoding a localization sequence which is operably linked to one or more coding sequences; an RNA cleavage agent recognition site, optionally wherein the RNA cleavage agent comprises a guide RNA and Cas nuclease, ribozyme, aptazyme, siRNA, ta-siRNA, or an miRNA; and/or an RNA molecule encoding a transcript-stabilizing or transcript-destabilizing sequence operably linked to the RNA molecule encoded by the DNA molecule.

65. The recombinant RNA molecule of any one of embodiments 47 to 64, wherein the RNA further comprises at least a segment of its reverse complementary RNA molecule.

66. An agricultural formulation comprising the recombinant RNA molecule of any one of embodiments 47 to 65.

67. The agricultural formulation of embodiment 66, wherein the recombinant RNA molecule is complexed with one or more RNA binding proteins or encapsidated by a viral capsid protein, optionally wherein the viral capsid protein is a partitiviral capsid protein and optionally wherein the RNA comprises an ERE which provides for encapsidation of the RNA by the partitiviral capsid protein.

68. The formulation of embodiment 66 or 67, wherein the RNA binding proteins comprise an RNA recognition motif.

69. The agricultural formulation of embodiment 66, 67, or 68, wherein the viral capsid protein is heterologous to the partitivirus.

70. The agricultural formulation of any one of embodiments 66 to 69, wherein the formulation comprises the recombinant RNA molecule and a carrier, an excipient, and/or an adjuvant.

71. A cell comprising the recombinant RNA molecule of any one of embodiments 47 to 65.

72. The cell of embodiment 71, wherein the cell is a bacterial cell, a fungal cell, a plant cell, or an animal cell.

73. An expression system comprising: (a) an RNA molecule comprising the recombinant RNA molecule of any one of embodiments 47 to 65; and (b) a cell containing the recombinant RNA molecule and an RDRP protein that recognizes the 5′ and 3′ RNA replication elements of the recombinant RNA molecule.

74. The expression system of embodiment 73, wherein the recombinant RNA molecule comprises an operably linked encapsidation recognition element (ERE) recognized by a viral capsid protein and the cell contains the viral capsid protein.

75. The expression system of embodiment 74, wherein the encapsidation recognition element (ERE) is a partitivirus ERE, wherein the viral capsid protein in the cell is a partitivirus capsid protein, and wherein the RNA molecule is encapsidated by the partitivirus capsid protein.

76. The expression system of any one of embodiments 73 to 75, further comprising the reverse complement of the recombinant RNA molecule.

77. The expression system of any one of embodiments 73 to 76, further comprising a RNA molecule comprising from 5′ to 3′ and operably linked: (i) a 5′ RNA replication element recognized by the partitiviral RNA-dependent RNA polymerase (RDRP); (ii) an RNA encoding a viral movement protein (MP); (iii) at least one tRNA-like molecule; and (iv) a 3′ RNA replication element recognized by the partitiviral RNA-dependent RNA polymerase (RDRP).

78. The expression system of any one of embodiments 73 to 77, further comprising a RNA molecule comprising from 5′ to 3′ and operably linked: (i) a 5′ RNA replication element recognized by the partitiviral RNA-dependent RNA polymerase (RDRP); (ii) an RNA encoding a viral movement protein (MP); (iii) at least one tRNA-like molecule; and (iv) a 3′ RNA replication element recognized by the partitiviral RNA-dependent RNA polymerase (RDRP).

79. The expression system of any one of embodiments 73 to 78, further comprising a RNA molecule comprising from 5′ to 3′ and operably linked: (i) a 5′ RNA replication element recognized by the partitiviral RNA-dependent RNA polymerase (RDRP); (ii) an RNA encoding a viral capsid protein (CP); (iii) at least one tRNA-like molecule; and (iv) a 3′ RNA replication element recognized by the partitiviral RNA-dependent RNA polymerase (RDRP).

80. The expression system of any one of embodiments 73 to 79, wherein the any one or all of the RNA molecules comprise an operably linked encapsidation recognition element (ERE), optionally wherein the ERE provides for encapsidation by the viral capsid protein encoded by the RNA molecule of embodiment 79.

81. The expression system of any one of embodiments 73 to 80, wherein the cell is a bacterial cell, a plant cell, a fungal cell, or an animal cell.

82. The expression system of embodiment 81, wherein the cell further comprises: (i) a viral capsid protein (CP), (ii) an RNA-binding protein (RBP) that can bind to the RNA molecule, optionally wherein the RBP binds to an RNA effecter; (iii) an RNA cleavage agent that cleaves the RNA molecule; (iv) an RNA promoter dependent RNA polymerase (RPDRP) protein that recognizes an RNA promoter in the RNA molecule; and/or (iv) a viral movement protein (MP).

83. The expression system of embodiment 82, wherein the CP, RBP, RPDRP, and/or the MP is: (a) expressed by a recombinant DNA molecule in the cell, (b) provided exogenously to the cell; (c) expressed by a recombinant RNA molecule in the cell; or (d) expressed by a virus in the cell.

84. The expression system of any one of embodiments 73 to 83, wherein the RDRP, CP, RBP, RPDRP, and/or the MP protein is heterologous to the cell.

85. The expression system of any one of embodiments 73 to 84, wherein the RDRP protein or a polynucleotide encoding the RDRP protein is provided exogenously to the cell.

86. The expression system of any one of embodiments 73 to 85, wherein the cell is a plant cell.

87. The expression system of any one of embodiments 73 to 86, wherein the RNA molecule further comprises an RNA promoter recognized by an RNA promoter dependent RNA polymerase (RPDRP) and operably linked to the RNA molecule,

88. The expression system of embodiment 87, wherein the cell further comprises an RNA promoter dependent RNA polymerase (RPDRP) that recognizes the RNA promoter and/or a polynucleotide encoding the RPDRP.

89. The expression system of any one of embodiments 73 to 88, wherein the plant cell contains a partitivirus which expresses the RDRP protein that recognizes the 5′ RNA replication element and the 3′ RNA replication element.

90. The expression system of embodiment 89, wherein the partitivirus occurs naturally in the plant cell.

91. A method of providing a synthetic partitiviral satellite RNA to a plant, comprising contacting the plant with the recombinant DNA molecule of any one of embodiments 1 to 23 or a formulation thereof, the cell of embodiment 25 or a formulation thereof, the vector of any one of embodiments 26 to 29 or a formulation thereof, the recombinant RNA molecule of any one of embodiments 47 to 65, or the formulation of any one of embodiments 66 to 69.

92. The method of embodiment 91, wherein contacting comprises spraying, dusting, injecting, or soaking with the recombinant DNA molecule, the cell, the vector, the recombinant RNA molecule, or the formulation thereof.

93. A method of establishing a synthetic partitivirus satellite RNA in a plant cell, comprising: providing to a plant cell the recombinant RNA molecule of any one of embodiments 47 to 64, and wherein the plant cell comprises an RDRP protein that recognizes the 5′ RNA replication element and 3′ RNA replication element, whereby the RDRP protein catalyzes synthesis of the synthetic partitivirus satellite RNA from the recombinant RNA molecule.

94. The method of embodiment 93, wherein the plant cell comprises a partitivirus and wherein the RDRP protein is provided to the plant cell by the partitivirus.

95. The method of embodiment 93 or 94, wherein the recombinant RNA molecule comprises an operably linked encapsidation recognition element (ERE) recognized by a viral capsid protein.

96. The method of embodiment 93, 94, or 95, wherein the plant cell comprises the viral capsid protein or a polynucleotide encoding the viral capsid protein.

97. The method of embodiment 93, 94, or 95, wherein the capsid protein comprises a partitiviral capsid protein and the ERE is recognized by the partitiviral capsid protein.

98. The method of any one of embodiments 93 to 97, wherein the partitivirus is endemic to the plant cell, optionally wherein the partitivirus which is endemic is non-pathogenic and/or commensal.

99. A method of obtaining a phenotypic change in a plant or plant cell, comprising: providing to a plant or plant cell the recombinant DNA molecule of any one of embodiments 1 to 23 or a formulation thereof, the cell of embodiment 25 or a formulation thereof, the vector of any one of embodiments 26 to 29 or a formulation thereof, the recombinant RNA molecule of any one of embodiments 47 to 64, or the formulation of any one of embodiments 66 to 69, wherein the cargo RNA molecule comprises RNA that effects a phenotypic change in the plant or plant cell in comparison to a plant or plant cell lacking the recombinant RNA, wherein the plant or plant cell comprises an RDRP protein that recognizes the 5′ RNA replication element and 3′ RNA replication element and catalyzes synthesis of a synthetic partitiviral RNA from the recombinant RNA molecule and the cargo RNA molecule effects the phenotypic change.

100. The method of embodiment 99, wherein the RNA that effects a phenotypic change in the plant or plant cell comprises an RNA for modulating a target gene's expression relative to the target gene's expression in a control plant or plant cell not provided with the recombinant RNA molecule, and wherein the phenotypic change is a result of the modulation.

101. The method of embodiment 99 or 100, wherein the modulation is (a) an increase of the target gene's expression; or (b) a decrease of the target gene's expression.

102. The method of embodiment 99, 100, or 101, wherein the RNA that effects a phenotypic change in the plant or plant cell suppresses the target gene's expression.

103. The method of any one of embodiments 99 to 102, wherein the RNA that effects a phenotypic change in the plant or plant cell comprises at least one RNA selected from an siRNA or siRNA precursor, a miRNA or miRNA precursor, and a phased siRNA or phased siRNA precursor.

104. The method of any one of embodiments 99 to 102, wherein the RNA that effects a phenotypic change in the plant or plant cell comprises a messenger RNA.

105. The method of embodiment 104, wherein the messenger RNA comprises an RNA molecule absent in the genome of the plant or plant cell.

106. The method of any one of embodiments 99 to 102, wherein the RNA that effects a phenotypic change in the plant or plant cell comprises an RNA for modifying the genome of the plant or plant cell.

107. The method of any one of embodiments 99 to 102, wherein the RNA that effects a phenotypic change in the plant or plant cell comprises an RNA for modifying the transcriptome and/or epigenome of the plant or plant cell.

108. The method of embodiment 107, wherein RNA for modifying the transcriptome targets an endogenous RNA of the plant which encodes an mRNA or a non-coding RNA for degradation.

109. The method of embodiment 108, wherein RNA for modifying the transcriptome comprises a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ligand-responsive ribozyme (aptazyme); an RNA aptamer; or a long noncoding RNA (lncRNA).

110. The method of embodiment 107, wherein the RNA for modifying the epigenome targets an endogenous plant gene for RNA-induced transcriptional silencing.

111. A method of increasing a plant's resistance to a pest or pathogen comprising providing a plant with the recombinant DNA molecule of any one of embodiments 1 to 23 or a formulation thereof, the cell of embodiment 25 or a formulation thereof, the vector of any one of embodiments 26 to 29 or a formulation thereof, the recombinant RNA molecule of any of embodiments 47 to 64, or the formulation of any one of embodiments 66 to 69, wherein the cargo RNA molecule effects an increase in the plant's resistance to a pest or pathogen in comparison to a plant lacking the recombinant RNA molecule, and wherein the plant or plant cell comprises an RDRP protein that recognizes the 5′ RNA replication element and the 3′ RNA replication element of the recombinant RNA and catalyzes synthesis of a synthetic partitiviral satellite RNA from the recombinant RNA molecule.

112. The method of embodiment 111, wherein the pest or pathogen is selected from the group comprising: a bacterium, a virus other than a partitivirus, a fungus, an oomycete, and an invertebrate.

113. The method of embodiment 111 or 112, wherein the cargo RNA molecule comprises an RNA that inhibits expression of a gene of the pest or pathogen and/or inhibits replication of the genome of the pest or pathogen.

114. The method of embodiment 111 or 112, wherein the cargo targets an endogenous RNA of the pest or pathogen for degradation.

115. The method of any one of embodiments 111 to 114, wherein cargo RNA comprises a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ligand-responsive ribozyme (aptazyme); an RNA aptamer; or a long noncoding RNA (lncRNA).

116. The method of any one of embodiments 111 to 115, wherein the cargo RNA targets an endogenous pest or pathogen gene for RNA-induced transcriptional silencing.

117. A method of increasing a plant's resistance to stress, comprising: providing a plant with the recombinant DNA molecule of any one of embodiments 1 to 23 or a formulation thereof, the cell of embodiment 25 or a formulation thereof, the vector of any one of embodiments 26 to 29 or a formulation thereof, the recombinant RNA molecule of any of embodiments 47 to 64, or the formulation of any one of embodiments 66 to 69, wherein the plant comprises an RDRP protein that recognizes the 5′ RNA replication element and the 3′ RNA replication element of the recombinant RNA and catalyzes synthesis of a synthetic partitiviral satellite RNA from the recombinant RNA molecule, and wherein the cargo RNA molecule effects an increase in the plant's resistance to stress relative to that in a plant lacking the recombinant RNA molecule.

118. The method of embodiment 117, wherein the stress comprises at least one abiotic stress comprising nutrient stress, light stress, water stress, heat stress, and/or cold stress.

119. The method of embodiment 117, wherein the stress comprises at least one biotic stress comprising crowding, shading, or allelopathy.

120. The method of embodiment 117, 118, or 119, wherein the cargo RNA molecule that effects an increase in the plant's resistance to stress comprises an RNA for modifying the transcriptome and/or epigenome of the plant or plant cell.

121. The method of embodiment 120, wherein RNA for modifying the transcriptome targets an endogenous RNA of the plant which encodes an mRNA or a non-coding RNA for degradation.

122. The method of embodiment 120 or 121, wherein RNA for modifying the transcriptome comprises a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ligand-responsive ribozyme (aptazyme); an RNA aptamer; or a long noncoding RNA (lncRNA).

123. The method of embodiment 120, wherein the RNA for modifying the epigenome targets an endogenous plant gene for RNA-induced transcriptional silencing.

124. The method of any one of embodiments 93 to 123, wherein the recombinant RNA molecule is provided to the plant of plant cell in the form of an RNA, an encapsidated RNA, or a formulation thereof.

125. The method of embodiment 124, wherein the encapsidated RNA comprises a synthetic partitiviral satellite particle.

126. The method of any one of embodiments 93 to 125, wherein the providing comprises contacting the plant or plant cell with the RNA, encapsidated RNA, or formulation thereof, optionally wherein contacting comprises spraying, dusting, injecting, or soaking the plant or plant cell with the RNA, encapsidated RNA, or formulation thereof.

127. The method of any one of embodiments 93 to 126, wherein the recombinant RNA further comprises its reverse complementary RNA molecule or a segment thereof.

128. A method of producing an exogenous polypeptide in a plant or plant cell, comprising: providing a plant or plant cell comprising the recombinant DNA molecule of any one of embodiments 1 to 18 or 20 to 23, wherein the cargo RNA molecule encoded by the DNA molecule comprises a translatable messenger RNA encoding the exogenous polypeptide, wherein the plant or plant cell comprises an RDRP protein that recognizes the 5′ RNA replication element and the 3′ RNA replication element of the recombinant RNA and that catalyzes synthesis of a synthetic partitiviral satellite RNA from the recombinant RNA molecule, and wherein the exogenous polypeptide is translated from the translatable messenger.

129. The method of any one of embodiments 91 to 127, further comprising a first step of providing a population of plants comprising the plant cells comprising the partitivirus which provides the RDRP protein; and then providing the recombinant DNA molecule, the cell, the vector. the recombinant RNA molecule, or formulation thereof to the plants comprising the plant cells.

130. The method of embodiment 129, wherein the partitivirus is exogenously provided to the plant cell.

131. The method of embodiment 129, wherein the RDRP protein or a recombinant polynucleotide encoding the RDRP is exogenously provided to the plant cell.

132. The method of embodiment 129, 130, or 131, further comprising a first step of providing a population of plants comprising the plant cells comprising the RDRP protein or the recombinant polynucleotide encoding the RDRP; and then providing the recombinant RNA molecule to the plants comprising the plant cells.

133. The method of any one of embodiments 129 to 132, wherein the recombinant RNA molecule has been produced in a fermentation system.

134. The method of any one of embodiments 91 to 133, further comprising the step of determining if the plant cell comprises a partitivirus which can provide the RDRP.

135. The method of embodiment 134, wherein it is determined that the plant cell comprises the partitivirus which can provide the RDRP, optionally wherein the partitivirus is not exogenously provided to the plant cell, the RDRP protein or the recombinant polynucleotide encoding the RDRP is not exogenously provided to the plant cell, or optionally wherein any combination of the partitivirus, RDRP protein, or polynucleotide encoding the RDRP is not exogenously provided to the plant cell.

136. The method of embodiment 134 or 135, wherein it is determined that the plant cell does not comprise the partitivirus which can provide the RDRP and the partitivirus is exogenously provided to the cell, the RDRP protein or the recombinant polynucleotide encoding the RDRP is exogenously provided to the plant cell, or a combination of the partitivirus, RDRP protein, or polynucleotide encoding the RDRP is exogenously provided to the plant cell.

137. The method of any one of embodiments 91 to 136, wherein the recombinant RNA molecule comprises an operably linked encapsidation recognition element (ERE) recognized by a viral capsid protein.

138. The method of embodiment 137, wherein the plant cell comprises the viral capsid protein or a polynucleotide encoding the viral capsid protein.

139. The method of embodiment 138, wherein the capsid protein comprises a partitiviral capsid protein and the ERE is recognized by the partitiviral capsid protein.

140. A method of manufacturing a synthetic partitiviral satellite particle, comprising: (a) providing to a plant cell the recombinant RNA molecule of any one of embodiments 47 to 64, wherein the recombinant RNA molecule comprises an encapsidation recognition element (ERE), and wherein the plant cell comprises an RDRP protein that recognizes the 5′ RNA replication element and 3′ RNA replication element catalyzes synthesis of a synthetic partitiviral satellite RNA from the recombinant RNA molecule and a viral capsid protein, wherein the ERE provides for encapsidation of the RNA by the viral capsid protein; and optionally (b) isolating the synthetic partitiviral satellite particle from the plant cell, a plant comprising the plant cell, or from media in which the plant cell or the plant had been grown.

141. The method of embodiment 140, wherein the RDRP and/or the viral capsid protein is: (a) expressed by a recombinant DNA molecule in the cell, (b) provided exogenously to the cell; (c) expressed by a recombinant RNA molecule in the cell; or (d) expressed by a virus in the cell.

142. The method of embodiment 140, wherein the plant cell comprises a partitivirus which expresses the RDRP protein and/or the viral capsid protein.

143. The method of embodiment 142, wherein the partitivirus occurs naturally in the plant cell.

144. The method of embodiment 140, 142, or 143, wherein the partitivirus is provided to the plant cell.

145. The method of any one of embodiments 140 to 144, wherein the recombinant RNA molecule is provided to the plant cell by a recombinant DNA construct comprising a promoter functional in the plant cell and operably linked to a DNA molecule encoding the recombinant RNA molecule, optionally wherein the recombinant DNA construct further comprises a DNA molecule encoding the RDRP and/or viral capsid protein.

146. The method of any one of embodiments 140 to 145, further comprising the step of purifying the synthetic partitiviral satellite particle.

147. The method of any one of embodiments 140 to 146, further comprising the step of formulating the synthetic partitiviral satellite particle.

148. The method of embodiment 147, wherein the formulating comprises combining the synthetic partitiviral satellite particle with a carrier, an excipient and/or an adjuvant.

149. A method of manufacturing a synthetic partitiviral satellite particle, comprising combining the recombinant RNA molecule of any one of embodiments 47 to 64 with a viral capsid protein in a vessel, wherein the recombinant RNA molecule comprises an encapsidation recognition element (ERE), and wherein the ERE provides for encapsidation of the RNA by the viral capsid protein in the vessel; optionally wherein the method further comprises isolating the synthetic partitiviral satellite particle from uncombined RNA and/or viral capsid protein in the vessel.

150. The method of embodiment 149, further comprising the steps of isolating the synthetic partitiviral satellite particle from uncombined RNA and/or viral capsid protein in the vessel and purifying the isolated the synthetic partitiviral satellite particle.

151. The method of embodiment 149 or 150, further comprising the step of purifying the isolated the synthetic partitiviral satellite particle.

152. The method of embodiment 149, 150, or 151 further comprising the step of formulating the synthetic partitiviral satellite particle.

153. The method of embodiment 152, wherein the formulating comprises combining the synthetic partitiviral satellite particle with a carrier, an excipient and/or an adjuvant.

154. The method of any one of embodiments 149 to 153, wherein the recombinant RNA further comprises its reverse complementary RNA molecule or at least a segment thereof.

155. A synthetic partitiviral satellite particle made by the method of any one of embodiments 140 to 153.

156. An agricultural formulation comprising the synthetic partitiviral satellite particle made by the method of any one of embodiments 140 to 153.

157. A method of providing a synthetic partitiviral satellite particle to a plant, comprising contacting the plant with the synthetic partitiviral satellite particle of embodiment 155 or the formulation of embodiment 156.

158. The method of embodiment 157, wherein contacting comprises spraying, dusting, injecting, or soaking with the synthetic partitiviral satellite particle or the formulation.

159. A plant propagule comprising the recombinant RNA molecule of any one of embodiments 47 to 65 and a partitiviral RDRP.

160. The plant propagule of embodiment 159, wherein the plant propagule is a seed.

161. The plant propagule of embodiment 159, wherein the plant propagule comprises a seedling, root, stem, leaf, shoot, tuber, rhizome, stolon, bulb, explant, embryo, or callus.

162. A plant propagule comprising at least one plant cell of embodiment 72 and a partitiviral RDRP.

163. The plant propagule of embodiment 162, wherein the plant propagule comprises a cell, or a seed, seedling, ovule, embryo, pollen, root, stem, leaf, shoot, tuber, rhizome, stolon, bulb, explant, or callus comprising the cell.

164. The plant propagule of embodiment 162, wherein the plant propagule is a seed.

165. The plant propagule of embodiment 162, 163, or 164, wherein the propagule is a mosaic comprising both plant cells comprising the partitiviral RDRP and plant cells lacking the partitiviral RDRP.

166. The plant propagule of any one of embodiments 162 to 165, wherein the plant propagule is a mosaic comprising both plant cells comprising the recombinant RNA molecule and plant cells lacking the recombinant RNA molecule.

167. The plant propagule of any one of embodiments 162 to 166, wherein the plant propagule lacks DNA encoding the recombinant RNA molecule.

168. A method of producing a modified plant propagule that comprises at least one plant cell comprising a recombinant RNA molecule of embodiment 47 to 65, comprising isolating a plant propagule comprising at least one plant cell comprising the recombinant RNA molecule and a partitiviral RNA-dependent RNA polymerase (RDRP) from a mixed population of plant cells comprising both plant cells comprising the recombinant RNA molecule and plant cells lacking the recombinant RNA molecule.

169. The method of embodiment 168, wherein the mixed population of plant cells comprise a population of protoplasts or a population of cells in callus or an explant.

170. The method of embodiment 168 or 169, wherein the mixed population of plant cells comprise plant cells comprising the partitiviral RDRP and plant cells lacking the partitiviral RDRP.

171. The method of embodiment 168 or 169, wherein the mixed population of plant cells comprise plant cells comprising the partitiviral RDRP.

172. The method of any one of embodiments 168 to 171, wherein the mixed population of plant cells is screened or selected for the presence of the plant cell comprising the recombinant RNA molecule prior to isolating the plant propagule.

173. The method of any one of embodiments 168 to 172, wherein the isolation comprises selecting for the plant cell comprising the recombinant RNA molecule prior to isolating the plant propagule.

174. The method of any one of embodiments 168 to 173, wherein the mixed population is located within a plant or a plant part.

175. The method of embodiment 174, wherein the plant or plant part is screened or selected for presence of the recombinant RNA molecule prior to isolating the plant propagule.

176. The method of embodiment 174, wherein the plant or plant part is screened or selected for systemic presence of the recombinant RNA molecule prior to isolating the plant propagule.

177. The method of any one of embodiments 168 to 176, wherein the recombinant RNA molecule encodes a selectable marker and the plant propagule comprising the recombinant RNA molecule is isolated by selecting for presence of the selectable marker.

178. The method of embodiment 177, further comprising selecting a plant propagule comprising a recombinant RNA molecule wherein the selectable marker has been removed.

179. The method of any one of embodiments 168 to 176, wherein the plant propagule comprising the recombinant RNA molecule is isolated by detecting the RNA molecule in one or more plant cells comprising the recombinant RNA molecule and separating the one or more plant cells comprising the recombinant RNA molecule from the plant cells lacking the recombinant DNA molecule.

180. The method of any one of embodiments 168 to 179, wherein the plant propagule is a mosaic comprising both plant cells comprising the recombinant RNA molecule and plant cells lacking the recombinant RNA molecule.

181. The method of any one of embodiments 168 to 180, wherein the plant propagule is a mosaic comprising both plant cells comprising the partitiviral RDRP and plant cells lacking the partitiviral RDRP.

182. The method of any one of embodiments 168 to 181, wherein the plant propagule lacks DNA that encodes the recombinant RNA molecule.

183. The method of embodiment 174, wherein the plant or plant part lacks DNA that encodes the recombinant RNA molecule.

184. The method of any one of embodiments 168 to 183, wherein the plant propagule comprises the cell, or a seed, seedling, root, stem, leaf, shoot, tuber, rhizome, stolon, bulb, explant, or callus comprising the cell.

185. The method of embodiment 184, further comprising multiplying the cell, seed, seedling, root, stem, leaf, shoot, tuber, rhizome, stolon, bulb, explant, or callus to obtain progeny, wherein the progeny comprise the recombinant RNA molecule.

186. The method of embodiment 185, wherein the multiplying comprises culturing a plurality of explants obtained from the cell, seed, seedling, root, stem, leaf, shoot, tuber, rhizome, stolon, bulb, explant, or callus.

187. The method of any one of embodiments 168 to 186, wherein the isolated propagule comprises the cell and the method further comprises regenerating a plant, seedling, root, stem, leaf, shoot, tuber, rhizome, stolon, bulb, explant, or callus comprising the recombinant RNA from said cell.

188. The method of any one of embodiments 168 to 186, wherein the isolated propagule comprises callus and the method further comprises regenerating a plant, seedling, root, stem, leaf, shoot, tuber, rhizome, stolon, bulb, or explant comprising the recombinant RNA from said callus.

189. The method of embodiment 187 or 188, wherein a plant is regenerated and wherein the method further comprises recovering F1 seed or F1 progeny comprising the recombinant RNA from the plant.

190. A method of providing a synthetic partitiviral satellite RNA to a plant comprising: grafting a scion onto a rootstock comprising recombinant RNA molecule of any one of embodiments 47 to 65, wherein at least one cell of the rootstock and/or the scion comprises the partitiviral RDRP. 191. The method of embodiment 190, wherein the scion lacks the recombinant RNA molecule prior to grafting.

192. The method of embodiment 190 or 191, wherein the rootstock comprises the partitiviral RDRP.

193. The method of any one of embodiments 190 to 192, wherein the RDRP is provided by a partitivirus endemic to the rootstock, optionally wherein the partitivirus endemic to the rootstock is non-pathogenic and/or commensal.

194. The method of any one of embodiments 190 to 192, wherein the RDRP is exogenously provided to the rootstock.

195. The method of any one of embodiments 190 to 194, wherein the scion comprises the partitiviral RDRP.

196. The method of any one of embodiments 190 to 194, wherein the RDRP is provided by a partitivirus endemic to the scion and/or wherein the RDRP is exogenously provided to the scion, optionally wherein the partitivirus endemic to the scion is non-pathogenic and/or commensal.

197. A method for producing a plant that transmits a recombinant RNA molecule to progeny plants or seed comprising isolating an F₁ progeny plant or seed comprising at least one cell comprising a partitiviral RNA-dependent RNA polymerase (RDRP) and the recombinant RNA molecule of any one of embodiments 47 to 65 from a population of F₁ plants or seed obtained from a parent plant comprising the recombinant RNA molecule.

198. The method of embodiment 197, wherein the F1 progeny plant or seed comprising the cell is isolated by screening the population of F₁ plants or seed obtained from a parent plant for the presence of the recombinant RNA molecule and propagating the F₁ progeny plant or seed comprising the recombinant RNA molecule.

199. The method of embodiment 197, wherein the recombinant RNA molecule encodes a selectable marker and the F₁ progeny plant or seed comprising the recombinant RNA molecule is isolated by selecting the F₁ progeny plant or seed comprising the recombinant RNA molecule for presence of the selectable marker.

200. The method of any one of embodiments 197 to 199, wherein the F1 progeny plant or seed lack DNA that encodes the recombinant RNA molecule.

201. The method of any one of embodiments 197 to 200, wherein the parent plant lacks DNA that encodes the recombinant RNA molecule.

202. The method of embodiment 199, wherein the selected F₁ progeny plant transmits the recombinant RNA molecule to at least F₂ progeny.

203. The method of any one of embodiments 197 to 202, wherein the F1 progeny plant or seed population is obtained from a parent plant used as a pollen recipient.

204. The method of any one of embodiments 197 to 202, wherein the F1 progeny plant or seed population is obtained from a parent plant used as a pollen donor.

205. The method of any one of embodiments 197 to 202, wherein the F1 progeny plant or seed population is obtained by selfing the parent plant.

206. The method of any one of embodiments 197 to 205, wherein the parent plant or a part thereof comprising the plant cell is screened or selected for presence of the recombinant RNA molecule prior to isolating the F₁ progeny plant or seed.

207. The method of any one of embodiments 197 to 205, wherein the parent plant or one or more parts thereof are screened for systemic presence of the recombinant RNA molecule prior to isolating the F₁ progeny plants, optionally wherein the part comprises floral tissue or male or female reproductive tissue.

208. The method of any one of embodiments 197 to 207, wherein the pericarp of the parent plant is screened or selected for presence of the recombinant RNA molecule.

209. The method of any one of embodiments 197 to 207, wherein F₁ seeds are obtained from a parent plant selected for presence of the recombinant RNA molecule in pericarp tissue.

210. The method of any one of embodiments 197 to 209, wherein an F₁ seed of the parent plant is non-destructively screened for presence of the recombinant RNA molecule.

211. The method of embodiment 210, wherein the F1 seed of the parent plant is non-destructively screened by assaying maternally derived or endosperm tissue of the seed for the presence of the recombinant RNA molecule.

212. The method of any one of embodiments 197 to 211, further comprising introducing the recombinant RNA molecule or a polynucleotide encoding the recombinant RNA molecule into a plant cell and obtaining the parent plant comprising the recombinant RNA molecule from the plant cell.

213. The method of any one of embodiments 168-212, wherein the propagule, plant, plant part, scion, and/or rootstock comprises a heterologous viral coat protein which can encapsidate the recombinant RNA molecule and/or comprises the recombinant RNA molecule encapsidated by a heterologous viral coat protein.

214. A method of barcoding a plant, plant cell, progeny thereof, or part thereof comprising providing to the plant or plant cell the recombinant RNA molecule of any one of embodiments 47 to 65, wherein the cargo RNA of the recombinant RNA molecule comprises a barcode RNA molecule, and wherein the plant or plant cell comprises a partitiviral RNA-dependent RNA polymerase (RDRP).

215. The method of embodiment 214, wherein the barcode RNA molecule comprises a sequence that uniquely identifies the plant, plant cell, progeny thereof, or part thereof.

216. The method of embodiment 214 or 215, wherein the barcode RNA molecule comprises a sequence that is not present in the genome and/or transcriptome of a wild-type plant of the same species, a pathogen thereof, or a symbiont thereof.

217. The method of any one of embodiments 214-216, wherein the barcode RNA molecule comprises a random sequence.

218. The method of any one of embodiments 214-217, wherein the barcode RNA molecule comprises a forward primer binding site and a reverse primer binding site that is not present in the genome and/or transcriptome of a wild-type plant of the same species, a pathogen thereof, or a symbiont thereof.

219. The method of any one of embodiments 214-217, wherein the barcode RNA molecule is up to about 3.2 kb in length.

220. The method of any one of embodiments 214-219, wherein the barcode RNA molecule has a length of 10 to 3200 nucleotides, 20 to 1000 nucleotides, or 50 to 500 nucleotides, optionally wherein the barcode RNA molecule has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, or 3200 nucleotides.

221. The method of any one of embodiments 214-220, wherein the barcode RNA molecule comprises a non-protein coding sequence.

222. The method of any one of embodiments 214-221, wherein the plant transmits the recombinant RNA molecule to progeny.

223. The method of any one of embodiments 214-222, wherein the plant, plant cell, progeny thereof, or part thereof lacks DNA that encodes the recombinant RNA molecule.

224. The method of any one of embodiments 214-223, further comprising isolating an F₁ progeny plant or seed comprising at least one cell comprising the partitiviral RDRP and the recombinant RNA molecule.

225. The method of embodiment 224, wherein the F₁ progeny plant or seed is obtained from the plant used as a pollen recipient.

226. The method of embodiment 224, wherein the F₁ progeny plant or seed is obtained from the plant used as a pollen donor.

227. The method of any one of embodiments 224-227, wherein the F₁ progeny plant or seed is obtained by selfing the plant.

228. The method of any one of embodiments 214-227, further comprising propagating the plant or plant cell to obtain a plant part or a plant propagule comprising the barcode RNA molecule, optionally wherein said propagule comprises callus, tubers, and/or rootstock.

229. A method of identifying a barcoded plant, plant part, or plant cell, the method comprising screening for the presence of a barcode RNA molecule in the plant, plant part, or plant cell, wherein the plant, plant part, or plant cell comprises the recombinant RNA molecule of any one of embodiments 47 to 65, wherein the cargo RNA of the recombinant RNA molecule comprises the barcode RNA molecule.

230. The method of embodiment 229, wherein the barcode RNA molecule comprises a sequence that uniquely identifies the plant, plant part, or plant cell.

231. The method of embodiment 229 or 230, wherein the barcode RNA molecule comprises a sequence that is not present in the genome and/or transcriptome of a wild-type plant of the same species, a pathogen thereof, or a symbiont thereof.

232. The method of any one of embodiments 229-231, wherein the barcode RNA molecule comprises a random sequence.

233. The method of any one of embodiments 229-232, wherein the barcode RNA molecule comprises a forward primer binding site and a reverse primer binding site that is not present in the genome and/or transcriptome of a wild-type plant of the same species, a pathogen thereof, or a symbiont thereof.

234. The method of any one of embodiments 229-233, wherein the barcode RNA molecule is up to about 3.2 kb in length.

235. The method of any one of embodiments 229-233, wherein the barcode RNA molecule has a length of 10 to 5000 nucleotides, 20 to 1000 nucleotides, or 50 to 500 nucleotides, optionally wherein the barcode RNA molecule has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, or 5000 nucleotides.

236. The method of any one of embodiments 229-235, wherein the barcode RNA molecule comprises a non-protein coding sequence.

237. The method of any one of embodiments 229-236, wherein the method comprises obtaining a nucleic acid sample from the plant, plant part, or plant cell; and detecting the presence of the barcode RNA molecule in the sample.

238. The method of any one of embodiments 229-237, wherein the screening comprises amplification and/or sequencing of the barcode RNA molecule.

239. The method of any one of embodiments 229-238, wherein the screening comprises detecting the barcode RNA molecule with a hybridization probe that hybridizes to at least a portion of the barcode RNA molecule.

240. The method of embodiment 239, wherein the hybridization probe comprises a detectable label.

241. The method of any one of embodiments 229-240, wherein the plant part comprises a seed, seedling, ovule, embryo, pollen, root, stem, leaf, shoot, tuber, rhizome, stolon, bulb, explant, or callus.

242. The method of embodiment 241, wherein the seed is non-destructively screened for presence of the barcode RNA molecule.

243. The method of any one of embodiments 229-242, wherein the plant, plant part, or plant cell comprises a partitiviral RDRP.

244. A plant comprising the recombinant RNA molecule of any one of embodiments 47 to 65 and a partitiviral RDRP.

245. The plant of embodiment 244, wherein the plant is a monocot or a dicot plant.

246. The plant of embodiment 244 or 245, wherein the plant is of the family Asteraceae, Cucurbitaceae, Fabaceae, Poaceae, or Solanaceae.

247. The plant of any one of embodiments 244-246, wherein the plant lacks DNA that encodes the recombinant RNA molecule.

248. The plant of any one of embodiments 244-247, wherein the plant comprises a partitivirus, and wherein the partitiviral RDRP is provided to the plant cell by the Partitivirus, optionally wherein the partitiviral RDRP comprises a protein having at least 85%, 90%, 95%, 97%, or 99% sequence identity to SEQ ID NO: 272-290, 384, or 390.

249. The plant of any one of embodiments 244-248, wherein the partitivirus is endemic to the plant, optionally wherein the endemic partitivirus is non-pathogenic and/or commensal.

250. The plant of any one of embodiments 244-249, wherein the partitiviral RDRP, the 5′replication element, and/or the 3′ replication element are derived from a partitivirus comprising one or both of the partitiviral RDRP, 5′ replication element, and/or 3′ replication elements.

251. The plant of any one of embodiments 244-250, wherein the RDRP has at least 85%, 90%, 95%, 97%, 98%, or 99% identity to SEQ ID NO: 272-290, 384, or 390, wherein the 5′ replication element has at least 85%, 90%, 95%, 97%, 98%, or 99% identity to the RNA encoded by SEQ ID NO: 2-34, 70-88, 372, 374, 376, 378, 380, 382, or 386, and/or wherein the 3′ replication element has at least 85%, 90%, 95%, 97%, 98%, or 99% identity to the RNA encoded by SEQ ID NO: 35-68, 89-108, 373, 375, 377, 379, 381, 383, or 387.

252. The plant of any one of embodiments 244-251, wherein the plant is a grafted plant and wherein the rootstock and or scion of the grafted plant comprise at least one cell comprising the recombinant RNA and the partitiviral RDRP.

253. The plant of any one of embodiments 244-252, wherein the plant is not produced by an essentially biological process.

254. A partitiviral satellite system that is self-replicating when introduced into a plant or plant cell, comprising: (1) a recombinant partitiviral satellite RNA comprising, from 5′ terminus to 3′ terminus: (a) a 5′ RNA replication element recognized by a partitiviral RNA-dependent RNA polymerase (RDRP); (b) a cargo RNA molecule; and (c) a 3′ RNA replication element recognized by the RDRP; wherein the 5′ RNA replication element, the cargo RNA molecule, and the 3′ RNA replication element are operably linked, and wherein the promoter and the cargo RNA molecule are heterologous to the 5′ RNA replication element and the 3′ RNA replication element; and (2) an exogenous partitivirus that is capable of replication in the plant or plant cells and that encodes the partitiviral RDRP that recognizes the 5′ and 3′ replicase recognition sequences in the recombinant partitiviral satellite RNA.

255. The self-replicating partitiviral satellite system of embodiment 254, wherein the exogenous Partitivirus is endemic or native to a different species, variety, or germplasm of plant.

266. The plant propagule of any one of embodiments 159-167, wherein the plant propagule is not produced by an essentially biological process.

### Summary of Tables

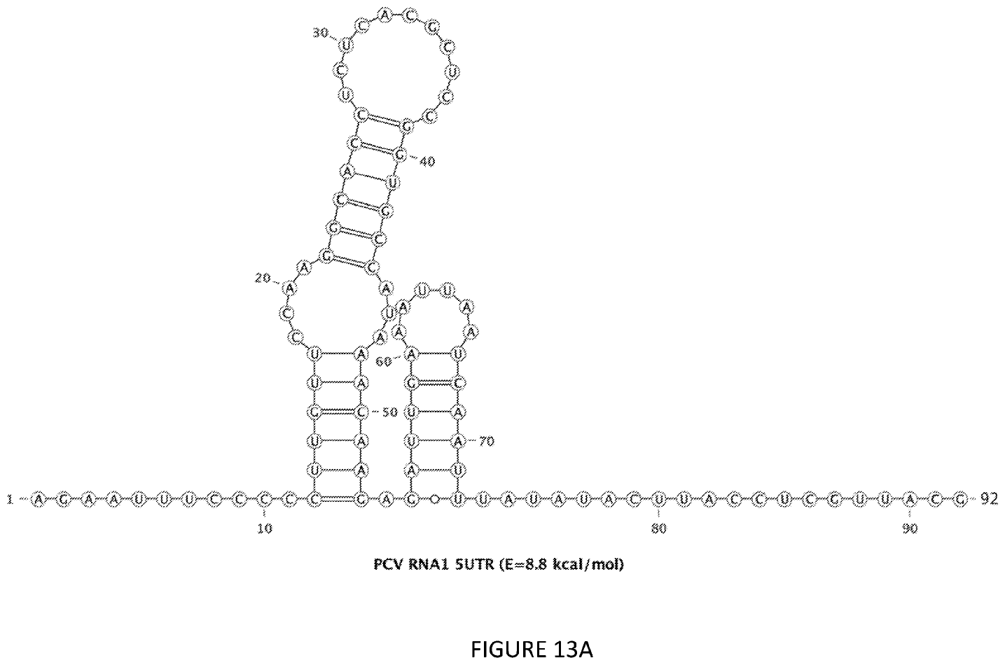

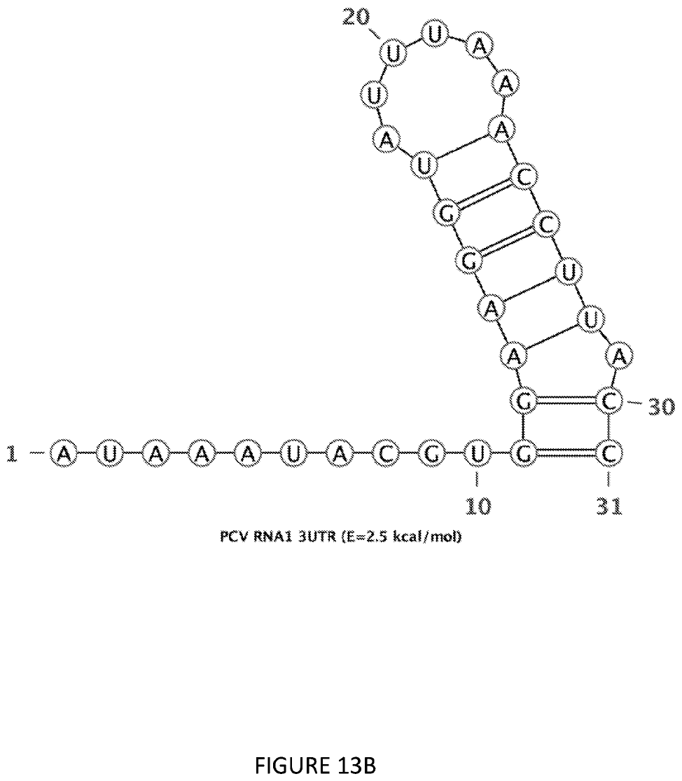

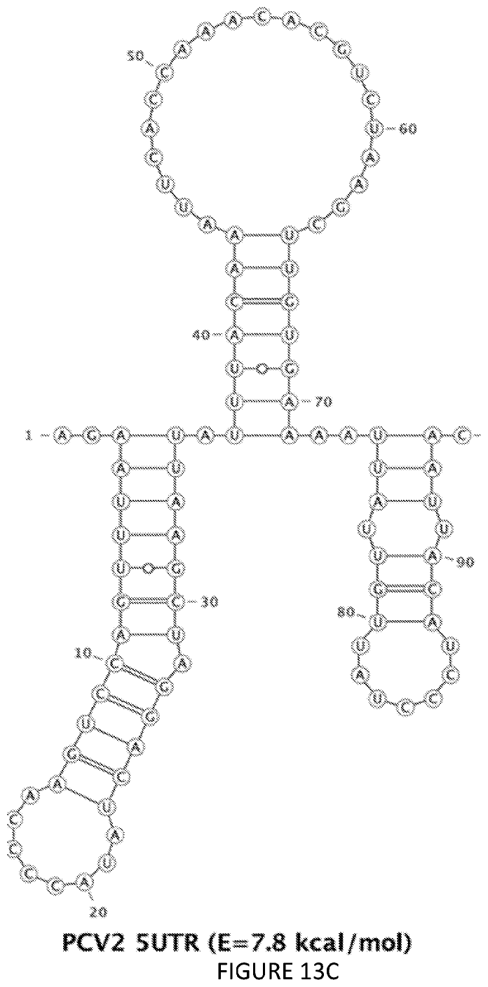

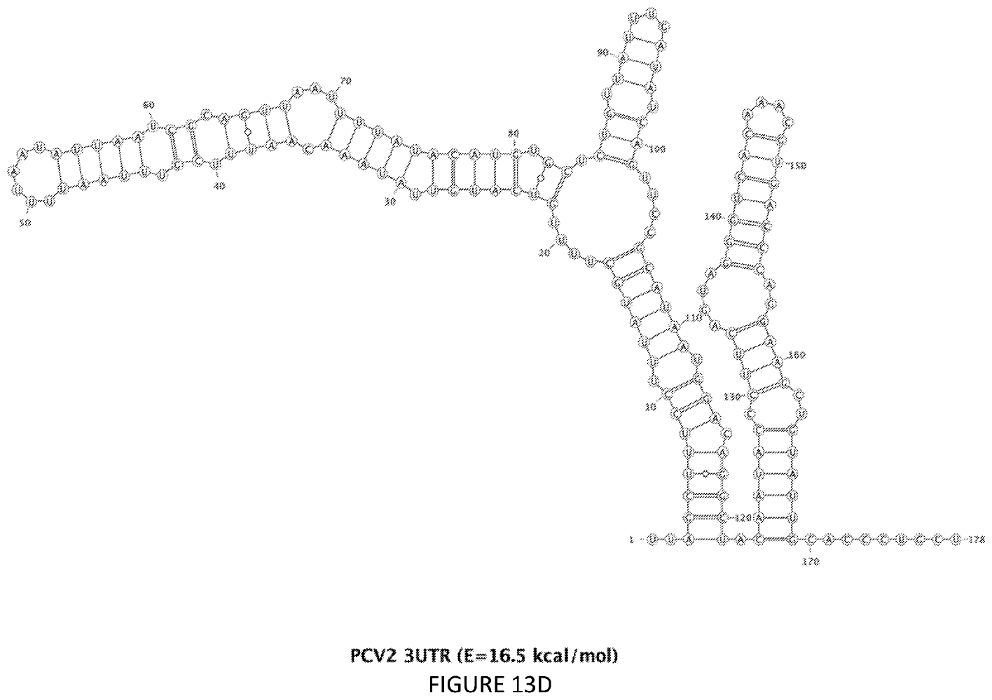

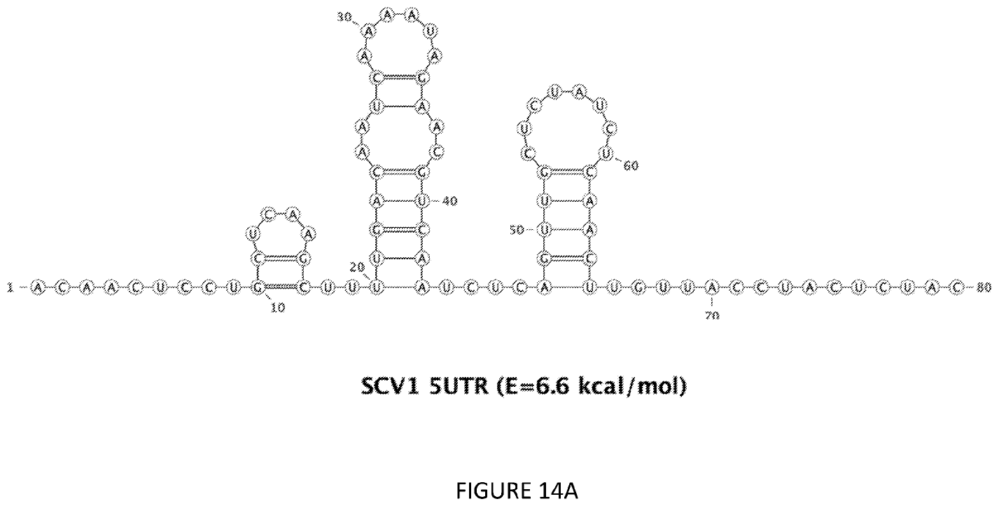

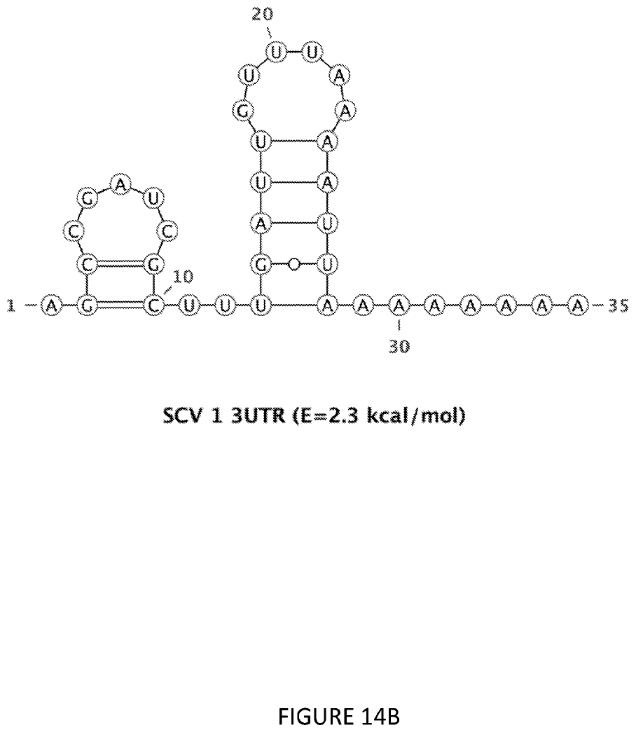

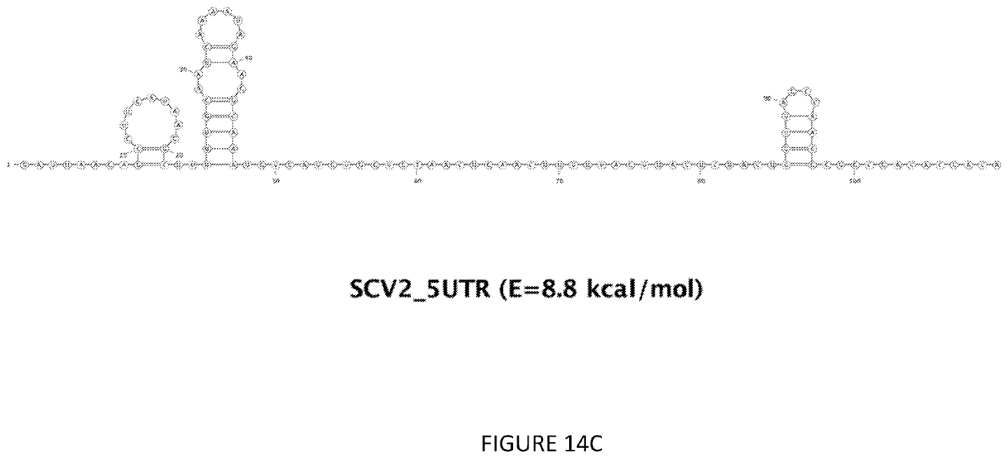

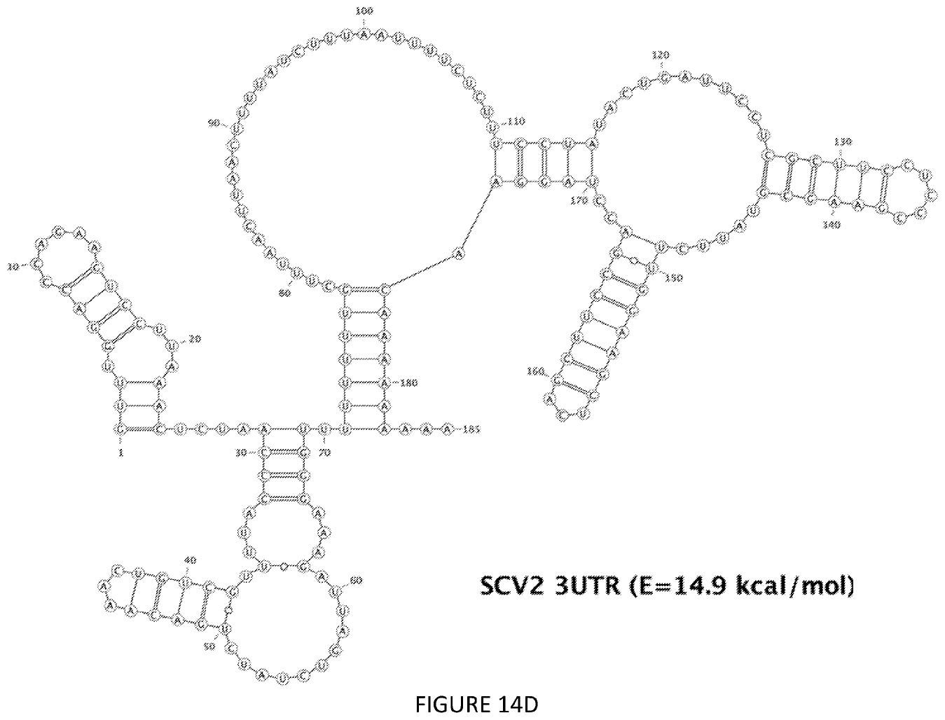

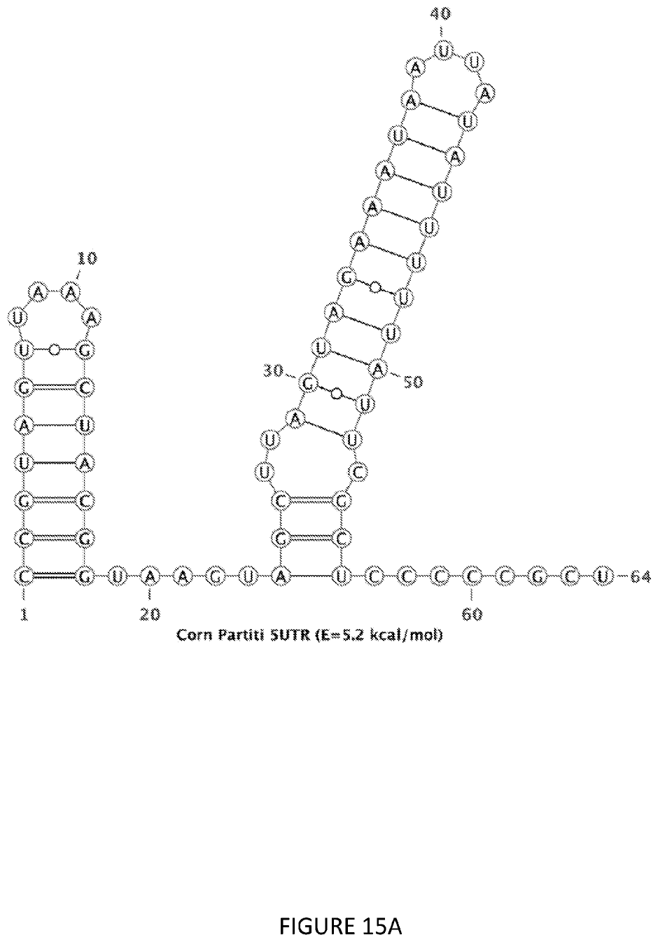

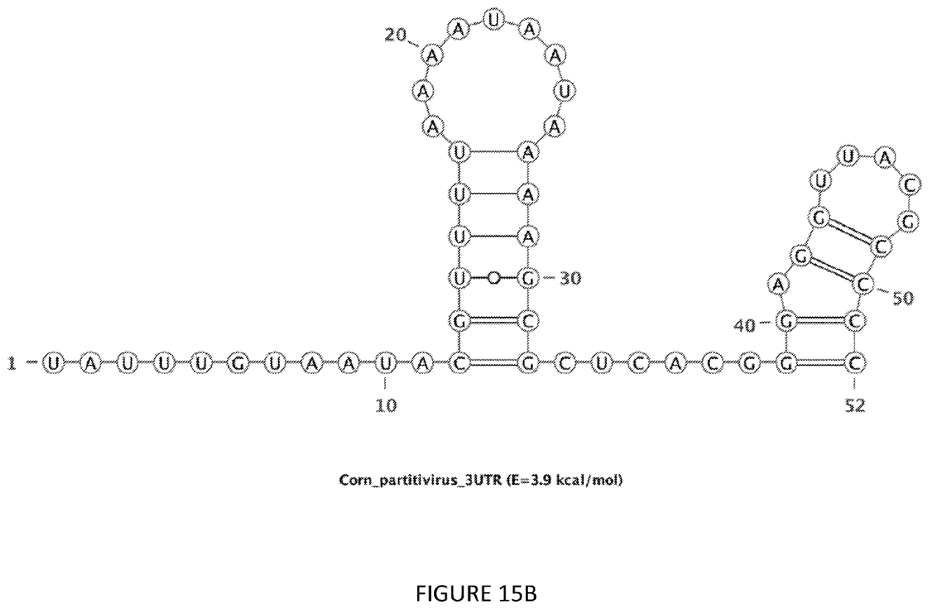

TABLE 1
Partitivirus 5′ and 3′ Replication Element DNA Coding Sequences

| | SEQ ID | |
|---|---|---|
| Description¹ | NO ² | Dot-Bracket structure of encoded RNA |
| PV Capsid Protein Genome-5′ | 1 | |
| Replication Element- Consensus | | |
| PV Capsid Protein Genome-5′ | 2 | ((((......))))..(((((..((......))..)))))................................ |
| Replication Element- NC_006276 | | ......((((...))))................ |
| PV Capsid Protein Genome-5′ | 3 | .......((((...(((((.............(((.(..((......))..).)))....))))))))) |
| Replication Element- NC_007242 | | .........................((((.....))))............ |
| PV Capsid Protein Genome-5′ | 4 | ((((((((((((((......)))))..)))))))))....................(((((......) |
| Replication Element- NC_008190 | | ))))...... |
| PV Capsid Protein Genome-5′ | 5 | ((((((((((........))))))))))........((((.(((((((((.((((.....)))). |
| Replication Element- NC_009520 | | )))).))))).)))).............(((((((...)))))))........(((((((....))) |
| | | ))))(((((.((((.(........).))))...))))).. |
| PV Capsid Protein Genome-5′ | 6 | (((((((((..(((....))).)))))))))((((((((((((....((((((....((((( |
| Replication Element- NC_009521 | | ........)))))........))))))).....))))....))))))))..(((((((((((.((( |
| | | (....((((((......)))))))))).)))...........))))))))............ |
| PV Capsid Protein Genome-5′ | 7 | (((((((((((.....)))))))))))..((.(((((.((((((((.((((...........(( |
| Replication Element- NC_010344 | | (((((.(((......))).)).))))))))).)))).))))(((.....)))......(((((.. |
| | | ...((.(((((........))))).)).....)))))....)))))))...((((.........)))) |
| | | ............ |
| PV Capsid Protein Genome-5′ | 8 | (((((((((((.....)))))))))))...(((((((((((((((((...((.((((((((.. |
| Replication Element- NC_010345 | | .))))))))))...))).)))))).....)).))))))....((((....))))((((((((... |
| | | .....(((((..(.(.....).)..)))))....)))).)))) |
| PV Capsid Protein Genome-5′ | 9 | ((((((((.((........)).))))))))((.(((((((((((.((((((((.(((....... |
| Replication Element- NC_010347 | | ...............))).))))))))......)))))).))))).)).....(((..((..(((... |
| | | .((((((......)))))))))))..)))...((((((....)).))))......... |
| PV Capsid Protein Genome-5′ | 10 | ((((((((((........)))))))))).......(((((.(((...(((((((........)))) |
| Replication Element- NC_010348 | | )))...))).)))))..(((..((((...(((......)))))))..))).(((((((((....) |
| | | ))))))))(((((((.....(((((...)))))..))))))) |
| PV Capsid Protein Genome-5′ | 11 | ((((....))))(((((...(((.(((.(((..(((((..((......))..)))))........) |
| Replication Element- NC_011557 | | )).)))..))).)))))....................(((((.......)))))... |
| PV Capsid Protein Genome-5′ | 12 | .(((((((..((((((..(((....))).)))))).)))))))...................((.... |
| Replication Element- NC_011706 | | .))....................... |
| PV Capsid Protein Genome-5′ | 13 | ....(((((((..(((....))).)))))))...(((((((..................))))))).. |
| Replication Element- NC_013110 | | .......................... |
| PV Capsid Protein Genome-5′ | 14 | ((((((((.((((((.((((...)))).))..(((.((((....(((.....((((.((..((( |
| Replication Element- NC_015495 | | (.(((....)))..))))..))...))))))))))).)))....(((((.(((....)))))))) |
| | | )).)).))))))))...................................... |
| PV Capsid Protein Genome-5′ | 15 | ..((((.((((.((((((.........)))))).)))).))))................ |
| Replication Element- NC_017988 | | |
| PV Capsid Protein Genome-5′ | 16 | .((..(((((((((((((...))))......)))))))))).............................. |
| Replication Element- NC_021095 | | ...................... |
| PV Capsid Protein Genome-5′ | 17 | .....((((((.((.((...)))).))))))....((((....)))).(((.((((((..(((... |
| Replication Element- NC_021097 | | ............)))...)))))).))) |
| PV Capsid Protein Genome-5′ | 18 | .....(((((..((((....)))))))))....((((....))))..............((((....) |
| Replication Element- NC_021099 | | ))).................... |
| PV Capsid Protein Genome-5′ | 19 | ..(((.(((((.((((............((((((.(((.(........)))).))))))...(((.. |
| Replication Element- NC_021148 | | .....)))...)))).))))).))).... |
| PV Capsid Protein Genome-5′ | 20 | ((((....))))..((((.......(((.(((((.........))))).))).......))))...(( |
| Replication Element- NC_022615 | | ((.......))))...................................... |
| PV Capsid Protein Genome-5′ | 21 | ...................................((((....))))............. |
| Replication Element- NC_022617 | | |
| PV Capsid Protein Genome-5′ | 22 | ((.((((((.((((.((.........)).)))).)))))).))............((((........) |
| Replication Element- NC_030245 | | ))).................... |
| PV Capsid Protein Genome-5′ | 23 | ((((((......))))))..........((((((..(((...((((((...))))))...)))))) |
| Replication Element- NC_030890 | | ))).................................. |
| PV Capsid Protein Genome-5′ | 24 | ((((((((((((((.((...)))))))).....))))))))...((((..................) |
| Replication Element- NC_031130 | | )))....................... |
| PV Capsid Protein Genome-5′ | 25 | ......((((.............(((((..((......))..))))).....))))................ |
| Replication Element- NC_033771 | | .............((((....)))).............. |
| | 378 | ........((.........))..(((((..((......))..))))).......................... |
| | (FIG. 14C) | ............((((....))))............. |
| PV Capsid Protein Genome-5′ | 26 | ..((((((((..((((((((((....))).)))))))....)))))))).................. |
| Replication Element- NC_034167 | | ............ |
| PV Capsid Protein Genome-5′ | 27 | .((((((((.((((((.(((((........))..))).)))))).))))....))))......... |
| Replication Element- NC_034523 | | ............................ |
| PV Capsid Protein Genome-5′ | 28 | ..((((((((((((........))))).)))))))..................((((........))) |
| Replication Element- NC_037096 | | )........(((((.........))))) |
| | 374 | ..((((((((((((........))))).))))))).(((((((.....................))) |
| | (FIG. 13C) | ))))..(((.(((.......))).))). |
| PV Capsid Protein Genome-5′ | 29 | ((((....))))..((((.......(((.(((((.........))))).))).......))))..... |
| Replication Element- NC_038823 | | .......(((((....((......))....)))))................ |
| PV Capsid Protein Genome-5′ | 30 | .....((((((.(((........))).))))))...((((((......(......)......)))))) |
| Replication Element- NC_038838 | | ............................ |
| PV Capsid Protein Genome-5′ | 31 | (((.((((..((((((((((((((....)).).))))))))))).)))))))............. |
| Replication Element- NC_038845 | | ..((((.((((((.....))))))........))))............ |
| PV Capsid Protein Genome-5′ | 32 | ..(((((((((((((((((..............)))))))))..)))..))))).............. |
| Replication Element- NC_038847 | | ................................((((((((......)))))...))).. |
| PV Capsid Protein Genome-5′ | 33 | .....................................((((....((((......)))).....))))...... |
| Replication Element- NC_040456 | | ................ |
| PV Capsid Protein Genome-5′ | 34 | ................(((((((((........).)))))))).................. |
| Replication Element- NC_040484 | | |
| PV Capsid Protein Genome-5′ | 380 | .....(((((..((((....)))))))))....((((....))))..............((((....) |
| Replication Element- JX971980 | | )))................... |
| PV Capsid Protein Genome-3′ | 35 | |
| Replication Element -consensus | | |
| PV Capsid Protein Genome-3′ | 36 | ......((((.......))))..........((..((.(((((......))))).))..)).(((((.. |
| Replication Element -NC_006276 | | ((((.....(((..((((.......)))).)))....)))).......)))))........(((....) |
| | | )) |
| PV Capsid Protein Genome-3′ | 37 | .....((((....(((((.((......))))))).....((((((...))))))........((((( |
| Replication Element -NC_007242 | | ((((...(((((........((((........)))).........)))))...))))....))))).( |
| | | (((((......))))))...............................)))).......... |
| PV Capsid Protein Genome-3′ | 38 | .....(((.......)))..........(((((((.((((((.....((((((..(((((....)))) |
| Replication Element -NC_008190 | | ).))))))...)))))).............(((((.....)))))......((((......)))).... |
| | | ....)))))))..(((((((...)))))))............................. |
| PV Capsid Protein Genome-3′ | 39 | .............(((((.............((((((......(((((....)))))....((((((... |
| Replication Element -NC_009520 | | (((((....)))))...)))))).................))))))...............((((((... |
| | | ...))))))(((((((..........)))))))(((.(((((.(((((((((..(((.(((((( |
| | | ((...))))))))..)))..)))))))))))))).)))....))))).. |
| PV Capsid Protein Genome-3′ | 40 | ...........((((((((((.......)))(((..(..((((((.((((.......)))).))))) |
| Replication Element -NC_009521 | | )..))))..(((((..((((((((...))))))))..)))))...)))))))..........((( |
| | | (.(((..((((((...)))))).((((((.((((......)))).)))))))))))))...... |
| | | (((.(((((((.((((.......)))).)).))))).)))...... |
| PV Capsid Protein Genome-3′ | 41 | ....((((((((((..((((.....(((....)))))))))))).).))))((((((((((... |
| Replication Element -NC_010344 | | ...).))))))))).(((((..((((((((((......)))).))))))(((((((.((..... |
| | | ..)).)))))))....((((........)))).((((.((((((.....))))))..))))...... |
| | | ...(((((((((((((((((((((..((((....))))..)))).....)))))))).))))). |
| | | ))))..))))).. |
| PV Capsid Protein Genome-3′ | 42 | .......(((((((..(((((((((..((......)).)))))))...))....)))))))(((( |
| Replication Element -NC_010345 | | (((........(((((.(((((....))))).))))).........((((((((...)))))))). |
| | | .)))))))................(((((((...)))))))........((((((((.(((.((((( |
| | | (.((((((((((((....))))).)))))(((((...))))).))))))))))).))))))) |
| | | ).......... |
| PV Capsid Protein Genome-3′ | 43 | ...((((((((((((.(((...(((((.....((.((((.(........).))))))))))).... |
| Replication Element -NC_010347 | | )))))))))))))))((((((((...))))))))......(((.(((((....(((.(((((( |
| | | ...........((((......))))(((((((..(((((......)))))...))))).))....... |
| | | )))))))))((((((.((.((((...)))).)).))).))))))))..))). |
| PV Capsid Protein Genome-3′ | 44 | ..............(((((..((((.........(((((..((((.........))))..).))))((( |
| Replication Element -NC_010348 | | (.........))))))))..)))))....(((......))).........((.(((((......)))) |
| | | ).))((((((.........))))))((((.(((((.((((((((((.....(((((((....)) |
| | | )))))...))))))))))))))).)))).... |
| PV Capsid Protein Genome-3′ | 45 | ..........((((.....((((.............((((.....))))...((((((..((...((((. |
| Replication Element -NC_011557 | | .......((((((............))))))...)))).))))))))((((((.....)))))).( |
| | | (((...((((.......))))..))))))))......)))).............. |
| PV Capsid Protein Genome-3′ | 46 | .....(((((.....)))..(((.....)))..((((.((.(.((....)).).)).))))....... |
| Replication Element NC_011706 | | ....(((((((.((((((.((.....)).))).)))...))))))).((((.....))))....(( |
| | | ((((....))))))(((((.((((((...((.......))...)))))))))))...((....)). |
| | | .(((((..(((((...((((((..((((......)))).))))))...).)))))))))....... |
| | | ...((((((((((....))))))))))))..(((((.((((...((((.(((((((((.....) |
| | | )))))))).....))))...))))...))))). |
| PV Capsid Protein Genome-3′ | 47 | ...........((((((.((((((((........))))))))((((....(((....)))...)))) |
| Replication Element NC_013110 | | ((((((((.....)))))))).(((((((((((......))).............((((((..((. |
| | | (((((((...))))))).))..)))))).........................................)) |
| | | ))))))........))))))..... |
| PV Capsid Protein Genome-3′ | 48 | .((((.........)))).............(((((((((((((((((.((.......)).))))).. |
| Replication Element NC_015495 | | )))))).................((((((...(.(((((((.........))))))).).))))))( |
| | | ((((.....)))))..........))))))(((.....((((((((((((((...)))))))))) |
| | | )))).....)))......... |
| PV Capsid Protein Genome-3′ | 49 | ........(((..(((((((((((((........(((...(..((((..((((((((........... |
| Replication Element NC_017988 | | .))))))))..))))..).)))...((((......))))))))))))))))))))((((...(( |
| | | ..((.(((((((.((........)).)))))))))..))...))))....... |
| PV Capsid Protein Genome-3′ | 50 | ..(((((..(((.....(((((((.......))))))).....)).)..)))))..............( |
| Replication Element NC_021095 | | (((((((.......)))).))))((((((((.(((((.....(((((((....)))))))(((( |
| | | (((........(((((((...)))))))........)))))))............................ |
| | | .....)))))))))))))............. |
| PV Capsid Protein Genome-3′ | 51 | (((..............((((((((...)))))))).....(((((....((((........)))).. |
| Replication Element NC_021097 | | ...)))))..))).....((((.((((((((..(((((((((..(((((......)))))(((( |
| | | (....)))))..(((((((....))))))).........................................) |
| | | )))))))))))))))))))).............. |
| PV Capsid Protein Genome-3′ | 52 | .((((((..(((....((((((......)))))).....)).)..))))))...............(( |
| Replication Element NC_021099 | | (((((((.....))))))))).((((((((..(((((...(((((((....)))))))((((. |
| | | ..))))...(((((.......)))))................................................ |
| | | .)))))..))))))))................ |
| PV Capsid Protein Genome-3′ | 53 | .((((((..(((((....(((((......)))))...)))).)..))))))...........((((( |
| Replication Element NC_021148 | | (((((((.....)))))).))))))((((((.((((((...))))))............(((((( |
| | | ...))))))..((((((.....))))))............................................. |
| | | ....))))))...................... |
| PV Capsid Protein Genome-3′ | 54 | ((..(((................))).))..........(((((....))))).........(((.....( |
| Replication Element NC_022615 | | (((((((((((((...((((....)))).....((((((((((((((................((( |
| | | (((((....(((.....)))....))))))))........)))))))))))))).............. |
| | | ...............)))))))))))))).......))).. |
| PV Capsid Protein Genome-3′ | 55 | .....((((......)))).........((((((((((((.......))))))((.(((....))). |
| Replication Element NC_022617 | | ))..(((((((((((((((.......)))................................(((((((.. |
| | | ..)))))))....))))))))))))..))))))................. |
| PV Capsid Protein Genome-3′ | 56 | ....((((...((((((((......))))))))))))....((((((((.....((((.(((...( |
| Replication Element NC_030245 | | ((((.((.....)).)))))...)))))))............((((((...))))))........... |
| | | ..(((..(((((..((((((((((......))).))))))).)))))))).....))))))))( |
| | | (.((..(((((((.((((((((((....))))))))))...))))))).)).))......... |
| PV Capsid Protein Genome-3′ | 57 | (.((((.((.((((......))))..)))))))...((((((((((((((.((...)).)))... |
| Replication Element NC_030890 | | ((((((((((..............(((((.....))))).))))))))))(..((((....)))). |
| | | .).........(((((((.............)))))))................))))))))))). |
| PV Capsid Protein Genome-3′ | 58 | .(((((...(((((...(((((...)))))....)))).)...)))))............((((((( |
| Replication Element NC_031130 | | (((((.....)))))).))))))......((((.......(((((.........)))))........) |
| | | )))................. |
| PV Capsid Protein Genome-3′ | 59 | ((((((((......))))..))))...(((((.(((((((((...)))))..........)))).) |
| Replication Element NC_033771 | | )))).......................................((((((...........((((((.....)) |
| | | )))).....((((((((...))))))))..)))))).......... |
| | 379 | (((.((((......))))...)))....((((...(.(((((...)))))...........)...))) |
| | (FIG. 14D) | ).(((((((.................................(((((...........((((((.....))) |
| | | ))).....((((((((...))))))))..))))).)))))))... |
| PV Capsid Protein Genome-3′ | 60 | ....((((((......((((.(.((((((((((((((((...)))))))))))....))))).) |
| Replication Element NC_034167 | | .)))).....))))))..(((...((((......)))))))((((((.((((((((((((..... |
| | | .))))..))))))))...)))))). |
| PV Capsid Protein Genome-3′ | 61 | .(((((((((((((.((((..........)))))))))....(((..((((.....))))..))) |
| Replication Element NC_034523 | | ))))))))...(((((((..(((.(((((.((((((((((.((((((.(..((((......)) |
| | | )).))))))).))))))).))).))))).)))....))))))).(((...(((((((.((... |
| | | ((((((((......))))))))..)))))))))....))).. |
| PV Capsid Protein Genome-3′ | 62 | .(((((((((.((((((....(((((((.(((((.((((.((.(((((......))))).)).) |
| Replication Element NC_037096 | | )))...)))))))))).)).(((.(((...))).)))....)))))).))).))))))((((( |
| | | (.((((....(((((((....)))))))..))))..))))))......... |
| | 375 | ..((((((((.((((((....(((((((.(((((.((((.((.(((((......))))).)).) |
| | (FIG. 13D) | )))...)))))))))).)).(((.(((...))).)))....)))))).))).))))).((((( |
| | | (.((((....(((((((....)))))))..))))..))))))......... |
| PV Capsid Protein Genome-3′ | 63 | ((....(((((.......)))))...))(((.((((...((((((....))))))......(((.( |
| Replication Element NC_038823 | | (((.(((.......(((((....................)))))........))))))).)))((((( |
| | | (....)))))))))).)))...........(((.((((....)))).))). |
| PV Capsid Protein Genome-3′ | 64 | ..........(((((((.(((((.....)))))....((((..(((...........)))))))((( |
| Replication Element NC_038838 | | (((((.....))))))))..((((((((.(((((.....((((((......))))))((((((( |
| | | .....((.((((((....)))))).)).....)))))))................................ |
| | | ))))).)))))))).....)))))))...... |
| PV Capsid Protein Genome-3′ | 65 | ((((((((((((((((((.....)))))))..............(((((((......))))))).. |
| Replication Element NC_038845 | | .(((((.(((((..((((........))))..)))))((((.........))))...((.....))) |
| | | )))).)))))))))))((((((..(((.((((..(((.(((((...))))).)))....)))) |
| | | ))))))))) |
| PV Capsid Protein Genome-3′ | 66 | (((((.(((....))).)))))..........((((.....(((..(((............)))..)) |
| Replication Element NC_038847 | | )...))))...(((((((((..((((((.......((((.....(((.((......)).)))..))) |
| | | )..)))))))).)))))))((((...(((((((((..((..((((......)))))..))..) |
| | | ))))).)))..)))) |
| PV Capsid Protein Genome-3′ | 67 | ..........(((...(((...((((......))))...)))....)))(((((..(((((((..(( |
| Replication Element NC_040456 | | (((.((((((((..(((((.............)))))))))))))))))).((.((((((..... |
| | | ........)))))).))((((((..(((((((((.......................))))..))))) |
| | | .)))))))))))))..)))))............... |
| PV Capsid Protein Genome-3′ | 68 | ..........((((((.....(((....((((((.((((((((.(((((..(((...(((......) |
| Replication Element NC_040484 | | ))..)))..).)))).))))....)))).))))))......(((((........))))))))..... |
| | | )))))).. |
| PV Capsid Protein Genome-3′ | 381 | .(((((((..(((((...(((((((....)))))))((((...))))...(((((.......))) |
| Replication Element JX971980 | | )).................................................)))))..)))))))......... |
| | | ........ |
| PV RdRP Genome-5′ Replication | 69 | |
| Element -consensus | | |
| PV RdRP Genome-5′ Replication | 70 | ...(((..((...(((((..((......))..)))))...))..)))........................ |
| Element -NC_006275 | | ... |
| PV RdRP Genome-5′ Replication | 71 | .......((((((((((((............((((...((......))...))))....)))))..... |
| Element -NC_007241 | | ..............))))))).. |
| PV RdRP Genome-5′ Replication | 72 | ((((((((((((((.....))))))...))))))))(((((((((..((((((.(((.(((( |
| Element -NC_009519 | | ((......)))))).))).)))))))))))))))......(((((((((((((.(....((((( |
| | | (...((((.(((((....)))))..))))...))))))...))))))))))).))) |
| PV RdRP Genome-5′ Replication | 73 | (((((((((((.....)))))))))))...(((....((((((.(((((((((.(((...((.. |
| Element -NC_010343 | | ....))...))))))))..)))).))))))....)))..((((((((((.(((..((((((((( |
| | | (((..((....))..)))))))))..)))..)))...))))))).))). |
| PV RdRP Genome-5′ Replication | 74 | (((((((((.((((.....))))....))))))))).((((((((...(((((((((.((((( |
| Element -NC_010346 | | (......)))))).)).))))))).)))))))).....(((((((((..((((.....(((((.. |
| | | ........(((((....))))).........)))))...))))..))))))))). |
| PV RdRP Genome-5′ Replication | 75 | ......((((((....))))))((...((...(((((..((......))..)))))...))...)).. |
| Element -NC_011556 | | .......................... |
| PV RdRP Genome-5′ Replication | 76 | ......((((.(((.........))).))))....(((((........)))))..((((((...((.( |
| Element -NC_011705 | | (((((....)))))).))....))))))..... |
| PV RdRP Genome-5′ Replication | 77 | (((((((..(((.(((..((..((.(((((((.(((...(((((.((((...(((((........ |
| Element -NC_015494 | | ......)))))..)))).....)))))...)))..))))))).)).))))))))..))))))).. |
| | | ............. |
| PV RdRP Genome-5′ Replication | 78 | (((((((..((((.(((((((((((.....((((((.....))))))..))))))....))))) |
| Element -NC_017989 | | .).)).)..)))))))..................... |
| PV RdRP Genome-5′ Replication | 79 | .............(((((.........(..((((((..((......))..))))))..))))))...... |
| Element -NC_022614 | | ....................... |
| PV RdRP Genome-5′ Replication | 80 | ..(((..((((((((((.......((.(((((..(.........)..)))))..)))))))))..) |
| Element -NC_030243 | | ))..)))..................(((......)))... |
| PV RdRP Genome-5′ Replication | 81 | ...................(((((..((......))..)))))....(((((........)))))...... |
| Element -NC_033770 | | .......... |
| | 376 | .........((....))..(((((..((......))..)))))....(((((........)))))..... |
| | (FIG. 14A) | .......... |
| PV RdRP Genome-5′ Replication | 82 | ((((((((((((((.((((.(((..((((((....))))))))))))).)))))))..)))) |
| Element -NC_034159 | | ))).............................. |
| PV RdRP Genome-5′ Replication | 83 | ....(((((((((((((((((........((((.....))))..)).)))))..)))))))))). |
| Element -NC_034513 | | .................(((...))).......... |
| PV RdRP Genome-5′ Replication | 84 | ...........((((((....((((((...........))))))...))))))(((((((......)) |
| Element -NC_037095 | | ))))).................... |
| | 372 | ...........((((((....((((((...........))))))...)))))).((((((......)) |
| | (FIG. 13A) | )))).................... |
| PV RdRP Genome-5′ Replication | 85 | ((((....))))..(((((.........(((.(((((.........))))).)))......))))).. |
| Element -NC_038824 | | .((((.......))))........... |
| PV RdRP Genome-5′ Replication | 86 | .(((((.(((((((((((((......(((....))).....))))..))))))))).)))))... |
| Element -NC_038846 | | ................................... |
| PV RdRP Genome-5′ Replication | 87 | ......(((((...)))))........((((......(((((((....)))))))...))))...... |
| Element -NC_040483 | | ...... |
| PV RdRP Genome-5′ Replication | 88 | ........((((((((...((((((.((.(((((((...))))))))).)))))).)))))..) |
| Element -NC_043394 | | ))((((((((......)))))))).............. |
| PV RdRP Genome-5′ Replication | 382 | .......(((((.((((...)))).)))))......((...........))..................... |
| Element -JX971980 | | ..................................... |
| PV RdRP Genome-5′ Replication | 386 | (((((((....))))))).....(((..((((((((((....)))))))))).)))........ |
| Element from Zea mays Wu312 | (FIG. 15A) | |
| PV RdRP Genome-3′ Replication | 89 | |
| Element -consensus | | |
| PV RdRP Genome-3′ Replication | 90 | .(((((.....)))))............... |
| Element -NC_006275 | | |
| PV RdRP Genome-3′ Replication | 91 | ((((.....))))..(((((((.(((((..................))))).)))))))......... |
| Element -NC_007241 | | .. |
| PV RdRP Genome-3′ Replication | 92 | ((((.((((....)))).))))......((((.....))))...((((((((((((........)) |
| Element -NC_009519 | | ))))))))))((((((((((........))))))))))........ |
| PV RdRP Genome-3′ Replication | 93 | .......((((....(((..((((.......))))..)))...))))(((((((((((((.((((( |
| Element -NC_010343 | | (((....))))).))).))))))))))))).......... |
| PV RdRP Genome-3′ Replication | 94 | .............((((((...............))))))............((((.(((((.........) |
| Element -NC_010346 | | ))))))))....(((((((((((...((((...)))))))))))))))........ |
| PV RdRP Genome-3′ Replication | 95 | ((((.....)))).(((((((((......................))))))))).......... |
| Element -NC_011556 | | |
| PV RdRP Genome-3′ Replication | 96 | .....................((((((....))))))..(((((..............))))) |
| Element -NC_011705 | | |
| PV RdRP Genome-3′ Replication | 97 | ..............(((((((.....)))))))..((((....(((((((((...)))))))))... |
| Element -NC_015494 | | .))))((.(((..(((..((((((((.((......))...))))))))...))))))))....... |
| | | .. |
| PV RdRP Genome-3′ Replication | 98 | .....(((((.......)))))((((((.....)))))).... |
| Element -NC_017989 | | |
| PV RdRP Genome-3′ Replication | 99 | (((......)))...((((((.((((((..................)))))).)))))).......... |
| Element -NC_022614 | | . |
| PV RdRP Genome-3′ Replication | 100 | ...................((.((.........)).)).... |
| Element -NC_030243 | | |
| PV RdRP Genome-3′ Replication | 101 | ...(((((.....)))))................. |
| Element -NC_033770 | 377 | .((.....))..(((((......)))))....... |
| | (FIG. 14B) | |
| PV RdRP Genome-3′ Replication | 102 | ..((((.((((((((.........................)))))))).))))(((((((.((..... |
| Element -NC_034159 | | .))))))))) |
| PV RdRP Genome-3′ Replication | 103 | ............(((.....)))(((.(((((....))))).))) |
| Element -NC_034513 | | |
| PV RdRP Genome-3′ Replication | 104 | ..........(((((((......))))).)) |
| Element -NC_037095 | 373 | ..........(((((((......))))).)) |
| | (FIG. 13B) | |
| PV RdRP Genome-3′ Replication | 105 | (((.....)))............... |
| Element -NC_038824 | | |
| PV RdRP Genome-3′ Replication | 106 | ............((((....)))).(((.((((((...)))))).))) |
| Element -NC_038846 | | |
| PV RdRP Genome-3′ Replication | 107 | ((((((((........))))))))..(((((..........)))))(((((....))))).. |
| Element -NC_040483 | | |
| PV RdRP Genome-3′ Replication | 108 | .....((((((..(((((...(((.......)))...)))))))))))(((((........))))) |
| Element -NC_043394 | | ... |
| PV RdRP Genome-3′ Replication | 383 | .((((((((......((((..(((.....)))..))))...................)))))))).... |
| Element -JX971980 | | ............ |
| PV RdRP Genome-3′ Replication | 387 | ...........((((((.........))))))......((.((.....)))) |
| Element Zea mays Wu312 | (FIG. 15B) | |

¹The descriptors “NC_XXXXXX” refer to the National Center for Biotechnology Information database accession number for entries in the world wide web internet database “ncbi.nlm.nih.gov/nuccore.”
² DNA sequences which encode the corresponding RNA molecules of the replication elements are provided are provided.

TABLE 2
Viral capsid protein and origin of assembly sequences

| | Origin of | |
|---|---|---|
| | assembly | Capsid |
| Virus¹ | sequence² | Protein |
| NC_001367.1 \| Tobacco mosaic virus, | SEQ ID | SEQ ID |
| complete genome\| Tobacco mosaic virus | NO: 109 | NO: 134 |
| NC_001556.1 \| Tobacco mild green mosaic | SEQ ID | SEQ ID |
| virus, complete genome\| Tobacco mild green | NO: 110 | NO: 135 |
| mosaic virus | | |
| NC_002692.1 \|Tomato mosaic virus, complete | SEQ ID | SEQ ID |
| genome\| Tomato mosaic virus | NO: 111 | NO: 136 |
| NC_003630.1 \|Pepper mild mottle virus, | SEQ ID | SEQ ID |
| complete genome\| Pepper mild mottle virus | NO: 112 | NO: 137 |
| NC_004106.1 \|Paprika mild mottle virus, | SEQ ID | SEQ ID |
| complete genome\|Paprika mild mottle virus | NO: 113 | NO: 138 |
| NC_009642.1 \|Bell pepper mottle tobamovirus, | SEQ ID | SEQ ID |
| complete genome\|Bell pepper mottle virus | NO: 114 | NO: 139 |
| NC_022230.1 \|Tomato mottle mosaic virus | SEQ ID | SEQ ID |
| isolate MX5, complete genome\|Tomato mottle | NO: 115 | NO: 140 |
| mosaic virus | | |
| NC_001728.1 \|Odontoglossum ringspot virus, | SEQ ID | SEQ ID |
| complete genome\|Odontoglossum ringspot virus | NO: 116 | NO: 141 |
| NC_003852.1 \|Obuda pepper virus, complete | SEQ ID | SEQ ID |
| genome\|Obuda pepper virus | NO: 117 | NO: 142 |
| NC_009041.1 \|Rehmannia mosaic virus, | SEQ ID | SEQ ID |
| complete genome\|Rehmannia mosaic virus | NO: 118 | NO: 143 |
| NC_010944.1 \|Brugmansia mild mottle virus, | SEQ ID | SEQ ID |
| complete genome\|Brugmansia mild mottle virus | NO: 119 | NO: 144 |
| NC_022801.1 \|Yellow tailflower mild mottle | SEQ ID | SEQ ID |
| virus isolate Cervantes, complete | NO: 120 | NO: 145 |
| genome\|Yellow tailflower mild mottle virus | | |
| NC_030229.1 \|Tropical soda apple mosaic | SEQ ID | SEQ ID |
| virus isolate Okeechobee, complete | NO: 121 | NO: 146 |
| genome\|Tropical soda apple mosaic virus | | |
| NC_001801.1 \|Cucumber green mottle mosaic | SEQ ID | SEQ ID |
| virus, complete genome\|Cucumber green mottle | NO: 122 | NO: 147 |
| mosaic virus | | |
| NC_002633.1 \|Cucumber fruit mottle mosaic | SEQ ID | SEQ ID |
| virus, complete genome\|Cucumber fruit mottle | NO: 123 | NO: 148 |
| mosaic virus | | |
| NC_003610.1 \|Kyuri green mottle mosaic | SEQ ID | SEQ ID |
| virus, complete genome\|Kyuri green mottle | NO: 124 | NO: 149 |
| mosaic virus | | |
| NC_003878.1 \|Zucchini green mottle mosaic | SEQ ID | SEQ ID |
| virus, complete genome\|Zucchini green mottle | NO: 125 | NO: 150 |
| mosaic virus | | |
| NC_008614.1 \|Cucumber mottle virus, | SEQ ID | SEQ ID |
| complete genome\|Cucumber mottle virus | NO: 126 | NO: 151 |
| watermelon green mottle mosaic virus | SEQ ID | SEQ ID |
| | NO: 127 | NO: 152 |
| NC_001873.1 \|Turnip vein-clearing virus, | SEQ ID | SEQ ID |
| complete genome\|Turnip vein-clearing virus | NO: 128 | NO: 153 |
| NC_002792.2 \|Ribgrass mosaic virus, complete | SEQ ID | SEQ ID |
| genome\|Ribgrass mosaic virus | NO: 129 | NO: 154 |
| NC_003355.1 \|Wasabi mottle virus, complete | SEQ ID | SEQ ID |
| genome\|Wasabi mottle virus | NO: 130 | NO: 155 |
| NC_004422.1 \|Youcai mosaic virus, complete | SEQ ID | SEQ ID |
| genome\|Youcai mosaic virus | NO: 131 | NO: 156 |
| NC_008365.1 \|Streptocarpus flower break | SEQ ID | SEQ ID |
| virus, complete genome\|Streptocarpus flower | NO: 132 | NO: 157 |
| break virus | | |
| NC_016442.1 \|Rattail cactus necrosis | SEQ ID | SEQ ID |
| associated virus, complete genome\|Rattail | NO: 133 | NO: 158 |
| cactus necrosis-associated virus | | |

¹The descriptors “NC_XXXXXX.1” refer to the National Center for Biotechnology Information database accession number for entries in the world wide web internet database “ncbi.nlm.nih.gov/nuccore.”
²RNA equivalents of the DNA sequence are also provided.

TABLE 3
Movement proteins.

| | Movement |
|---|---|
| | protein |
| Virus | (MP) |
| NC_001367.1 \|Tobacco mosaic virus, complete | SEQ ID |
| genome\|Tobacco mosaic virus | NO: 159 |
| NC_001556.1 \|Tobacco mild green mosaic virus, | SEQ ID |
| complete genome\|Tobacco mild green mosaic virus | NO: 160 |
| NC_002692.1 \|Tomato mosaic virus, complete | SEQ ID |
| genome\|Tomato mosaic virus | NO: 161 |
| NC_003630.1 \|Pepper mild mottle virus, complete | SEQ ID |
| genome\|Pepper mild mottle virus | NO: 162 |
| NC_004106.1 \|Paprika mild mottle virus, complete | SEQ ID |
| genome\|Paprika mild mottle virus | NO: 163 |
| NC_009642.1 \|Bell pepper mottle tobamovirus, | SEQ ID |
| complete genome\|Bell pepper mottle virus | NO: 164 |
| NC_022230.1 \|Tomato mottle mosaic virus isolate | SEQ ID |
| MX5, complete genome\|Tomato mottle mosaic virus | NO: 165 |
| NC_001728.1 \|Odontoglossum ringspot virus, | SEQ ID |
| complete genome\|Odontoglossum ringspot virus | NO: 166 |
| NC_003852.1 \|Obuda pepper virus, complete | SEQ ID |
| genome\|Obuda pepper virus | NO: 167 |
| NC_009041.1 \|Rehmannia mosaic virus, complete | SEQ ID |
| genome\|Rehmannia mosaic virus | NO: 168 |
| NC_010944.1 \|Brugmansia mild mottle virus, | SEQ ID |
| complete genome\|Brugmansia mild mottle virus | NO: 169 |
| NC_022801.1 \|Yellow tailflower mild mottle virus | SEQ ID |
| isolate Cervantes, complete genome/Yellow | NO: 170 |
| tailflower mild mottle virus | |
| NC_030229.1 \|Tropical soda apple mosaic virus | SEQ ID |
| isolate Okeechobee, complete genome\|Tropical | NO: 171 |
| soda apple mosaic virus | |
| NC_001801.1 \|Cucumber green mottle mosaic | SEQ ID |
| virus, complete genome\|Cucumber green mottle | NO: 172 |
| mosaic virus | |
| NC_002633.1 \|Cucumber fruit mottle mosaic | SEQ ID |
| virus, complete genome\|Cucumber fruit mottle | NO: 173 |
| mosaic virus | |
| NC_003610.1 \|Kyuri green mottle mosaic virus, | SEQ ID |
| complete genome\|Kyuri green mottle mosaic virus | NO: 174 |
| NC_003878.1 \|Zucchini green mottle mosaic virus, | SEQ ID |
| complete genome\|Zucchini green mottle mosaic virus | NO: 175 |
| NC_008614.1 \|Cucumber mottle virus, complete | SEQ ID |
| genome\|Cucumber mottle virus | NO: 176 |
| watermelon green mottle mosaic virus | SEQ ID |
| | NO: 177 |
| NC_001873.1 \|Turnip vein-clearing virus, | SEQ ID |
| complete genome\|Turnip vein-clearing virus | NO: 178 |
| NC_002792.2 \|Ribgrass mosaic virus, complete | SEQ ID |
| genome\|Ribgrass mosaic virus | NO: 179 |
| NC_003355.1 \|Wasabi mottle virus, complete | SEQ ID |
| genome\|Wasabi mottle virus | NO: 180 |
| NC_004422.1 \|Youcai mosaic virus, complete | SEQ ID |
| genome\|Youcai mosaic virus | NO: 181 |
| NC_008365.1 \|Streptocarpus flower break virus, | SEQ ID |
| complete genome\|Streptocarpus flower break virus | NO: 182 |
| NC_016442.1 \|Rattail cactus necrosis associated | SEQ ID |
| virus, complete genome\|Rattail cactus | NO: 183 |
| necrosis-associated virus | |

¹ The descriptors “NC_XXXXXX.1” refer to the National Center for Biotechnology Information database accession number for entries in the world wide web internet database “ncbi.nlm.nih.gov/nuccore.”

TABLE 4
tRNA-like sequences

| Name¹ of Arabidopsis gene | |
|---|---|
| containing tRNA-like | |
| sequence that can confer | |
| cell-to-cell mobility | Sequence² |
| >AT1G59870.1 | SEQ ID NO: 184 |
| >AT1G71697.1 | SEQ ID NO: 185 |
| >AT1G77885.1 | SEQ ID NO: 186 |
| >AT2G04400.1 | SEQ ID NO: 187 |
| >AT2G20230.1 | SEQ ID NO: 188 |
| >AT2G24790.1 | SEQ ID NO: 189 |
| >AT2G32540.1 | SEQ ID NO: 190 |
| >AT3G15850.1 | SEQ ID NO: 191 |
| >AT3G25770.1 | SEQ ID NO: 192 |
| >AT4G15570.1 | SEQ ID NO: 193 |
| >AT4G26050.1 | SEQ ID NO: 194 |
| >AT4G26050.1 | SEQ ID NO: 195 |
| >AT4G27430.2 | SEQ ID NO: 196 |
| >AT4G27430.2 | SEQ ID NO: 197 |
| >AT5G46330.1 | SEQ ID NO: 198 |
| >AT5G54110.1 | SEQ ID NO: 199 |
| >AT5G65730.1 | SEQ ID NO: 200 |
| >AT3G02020.1 | SEQ ID NO: 201 |
| >AT5G63840.1 | SEQ ID NO: 202 |
| >AT2G36930.1 | SEQ ID NO: 203 |
| >AT5G59950.5 | SEQ ID NO: 204 |
| >AT5G20180.1 | SEQ ID NO: 205 |
| >AT5G20180.2 | SEQ ID NO: 206 |
| >AT1G07940.2 | SEQ ID NO: 207 |
| >AT2G14260.2 | SEQ ID NO: 208 |
| >AT3G05520.2 | SEQ ID NO: 209 |
| >AT4G10840.1 | SEQ ID NO: 210 |
| >AT4G10840.2 | SEQ ID NO: 211 |
| >AT1G73650.3 | SEQ ID NO: 212 |
| >AT2G45990.1 | SEQ ID NO: 213 |
| >AT2G45990.3 | SEQ ID NO: 214 |
| >AT2G45990.4 | SEQ ID NO: 215 |
| >AT3G03160.1 | SEQ ID NO: 216 |
| >AT5G64870.1 | SEQ ID NO: 217 |
| >AT1G13600.1 | SEQ ID NO: 218 |
| >AT1G73177.1 | SEQ ID NO: 219 |
| >AT1G07670.1 | SEQ ID NO: 220 |
| >AT1G55490.1 | SEQ ID NO: 221 |
| >AT1G55490.2 | SEQ ID NO: 222 |
| >AT1G55680.1 | SEQ ID NO: 223 |
| >AT2G14120.1 | SEQ ID NO: 224 |
| >AT2G14120.2 | SEQ ID NO: 225 |
| >AT2G14120.3 | SEQ ID NO: 226 |
| >AT2G32730.1 | SEQ ID NO: 227 |
| >AT3G10760.1 | SEQ ID NO: 228 |
| >AT4G10320.1 | SEQ ID NO: 229 |
| >AT4G16990.4 | SEQ ID NO: 230 |
| >AT5G59380.1 | SEQ ID NO: 231 |

¹The descriptors “ATXXXXXXX.X” refer to the Arabidopsis Information Resource (TAIR) database accession number for entries in the world wide web internet database “arabidopsis.org.”

TABLE 5
IRES sequence

| IRES name | Sequence¹ |
|---|---|
| EMCV IRES | SEQ ID NO: 232 |
| CrTMV IRES | SEQ ID NO: 233 |
| HCRSV IRES | SEQ ID NO: 234 |
| ZmHSP101 IRES | SEQ ID NO: 235 |

¹DNA equivalents of the RNA sequence are also provided.

TABLE 6
Intron sequences

| Intron | |
|---|---|
| number | Sequence |
| 1 | SEQ ID NO: 236 |
| 2 | SEQ ID NO: 237 |
| 3 | SEQ ID NO: 238 |
| 4 | SEQ ID NO: 239 |
| 5 | SEQ ID NO: 240 |
| 6 | SEQ ID NO: 241 |
| 7 | SEQ ID NO: 242 |
| 8 | SEQ ID NO: 243 |
| 9 | SEQ ID NO: 244 |
| 10 | SEQ ID NO: 245 |
| 11 | SEQ ID NO: 246 |
| 12 | SEQ ID NO: 247 |
| 13 | SEQ ID NO: 248 |
| Potato | SEQ ID NO: 391 |
| ST-LS1 | |
| intron2 | |

TABLE 7
Partitiviral CP Genomes and CP sequences

| | Genomic Sequence | | |
|---|---|---|---|
| | or 5′ replication | CP Protein | CP DNA coding |
| PV Capsid Protein (CP) Genome | element sequence | Sequence | sequence |
| PV Capsid Protein Genome - NC_006276 | SEQ ID NO: 260 | SEQ ID NO: 268 | SEQ ID NO: 310 |
| PV Capsid Protein Genome-NC_007242 | SEQ ID NO: 261 | SEQ ID NO: 269 | SEQ ID NO: 311 |
| PV Capsid Protein Genome-NC_008190 | SEQ ID NO: 262 | SEQ ID NO: 270 | SEQ ID NO: 312 |
| PV Capsid Protein Genome - NC_009520 | SEQ ID NO: 263 | SEQ ID NO: 271 | SEQ ID NO: 313 |
| PV Capsid Protein - NC_009521 | SEQ ID NO: 6 | SEQ ID NO: 314 | SEQ ID NO: 343 |
| PV Capsid Protein -NC_010344 | SEQ ID NO: 7 | SEQ ID NO: 315 | SEQ ID NO: 344 |
| PV Capsid Protein -NC_010345 | SEQ ID NO: 8 | SEQ ID NO: 316 | SEQ ID NO: 345 |
| PV Capsid Protein -NC_010347 | SEQ ID NO: 9 | SEQ ID NO: 317 | SEQ ID NO: 346 |
| PV Capsid Protein -NC_010348 | SEQ ID NO: 10 | SEQ ID NO: 318 | SEQ ID NO: 347 |
| PV Capsid Protein -NC_011557 | SEQ ID NO: 11 | SEQ ID NO: 319 | SEQ ID NO: 348 |
| PV Capsid Protein -NC_011706 | SEQ ID NO: 12 | SEQ ID NO: 320 | SEQ ID NO: 349 |
| PV Capsid Protein -NC_013110 | SEQ ID NO: 13 | SEQ ID NO: 321 | SEQ ID NO: 350 |
| PV Capsid Protein -NC_015495 | SEQ ID NO: 14 | SEQ ID NO: 322 | SEQ ID NO: 351 |
| PV Capsid Protein -NC_017988 | SEQ ID NO: 15 | SEQ ID NO: 323 | SEQ ID NO: 352 |
| PV Capsid Protein -NC_021095 | SEQ ID NO: 16 | SEQ ID NO: 324 | SEQ ID NO: 353 |
| PV Capsid Protein -NC_021097 | SEQ ID NO: 17 | SEQ ID NO: 325 | SEQ ID NO: 354 |
| PV Capsid Protein -NC_021099 | SEQ ID NO: 18 | SEQ ID NO: 326 | SEQ ID NO: 355 |
| PV Capsid Protein -NC_021148 | SEQ ID NO: 19 | SEQ ID NO: 327 | SEQ ID NO: 356 |
| PV Capsid Protein -NC_022615 | SEQ ID NO: 20 | SEQ ID NO: 328 | SEQ ID NO: 357 |
| PV Capsid Protein -NC_022617 | SEQ ID NO: 21 | SEQ ID NO: 329 | SEQ ID NO: 358 |
| PV Capsid Protein -NC_030245 | SEQ ID NO: 22 | SEQ ID NO: 330 | SEQ ID NO: 359 |
| PV Capsid Protein -NC_030890 | SEQ ID NO: 23 | SEQ ID NO: 331 | SEQ ID NO: 360 |
| PV Capsid Protein -NC_031130 | SEQ ID NO: 24 | SEQ ID NO: 332 | SEQ ID NO: 361 |
| PV Capsid Protein -NC_033771 | SEQ ID NO: 25 | SEQ ID NO: 333 | SEQ ID NO: 362 |
| | or 378 | | |
| PV Capsid Protein -NC_034167 | SEQ ID NO: 26 | SEQ ID NO: 334 | SEQ ID NO: 363 |
| PV Capsid Protein -NC_034523 | SEQ ID NO: 27 | SEQ ID NO: 335 | SEQ ID NO: 364 |
| PV Capsid Protein -NC_037096 | SEQ ID NO: 28 | SEQ ID NO: 336 | SEQ ID NO: 365 |
| | or 374 | | |
| PV Capsid Protein -NC_038823 | SEQ ID NO: 29 | SEQ ID NO: 337 | SEQ ID NO: 366 |
| PV Capsid Protein -NC_038838 | SEQ ID NO: 30 | SEQ ID NO: 338 | SEQ ID NO: 367 |
| PV Capsid Protein -NC_038845 | SEQ ID NO: 31 | SEQ ID NO: 339 | SEQ ID NO: 368 |
| PV Capsid Protein -NC_038847 | SEQ ID NO: 32 | SEQ ID NO: 340 | SEQ ID NO: 369 |
| PV Capsid Protein -NC_040456 | SEQ ID NO: 33 | SEQ ID NO: 341 | SEQ ID NO: 370 |
| PV Capsid Protein -NC_040484 | SEQ ID NO: 34 | SEQ ID NO: 342 | SEQ ID NO: 371 |

TABLE 8
Partitiviral RDRP sequences

| | Genomic Sequence | | |
|---|---|---|---|
| | or 5′ replication | RDRP Protein | RDRP DNA |
| PV RDRP Genome | element sequence | Sequence | Coding Sequence |
| PV RdRP Genome-NC_006275 | SEQ ID NO: 264 | SEQ ID NO: 272 | SEQ ID NO: 291 |
| PV RdRP Genome-NC_007241 | SEQ ID NO: 265 | SEQ ID NO: 273 | SEQ ID NO: 292 |
| PV RdRP Genome-NC_009519 | SEQ ID NO: 266 | SEQ ID NO: 274 | SEQ ID NO: 293 |
| PV RdRP Genome-NC_010343 | SEQ ID NO: 267 | SEQ ID NO: 275 | SEQ ID NO: 294 |
| PV RdRP-NC_010346 | SEQ ID NO: 74 | SEQ ID NO: 276 | SEQ ID NO: 295 |
| PV RdRP-NC_011556 | SEQ ID NO: 75 | SEQ ID NO: 277 | SEQ ID NO: 296 |
| PV RdRP-NC_011705 | SEQ ID NO: 76 | SEQ ID NO: 278 | SEQ ID NO: 297 |
| PV RdRP-NC_015494 | SEQ ID NO: 77 | SEQ ID NO: 279 | SEQ ID NO: 298 |
| PV RdRP-NC_017989 | SEQ ID NO: 78 | SEQ ID NO: 280 | SEQ ID NO: 299 |
| PV RdRP-NC_022614 | SEQ ID NO: 79 | SEQ ID NO: 281 | SEQ ID NO: 300 |
| PV RdRP-NC_030243 | SEQ ID NO: 80 | SEQ ID NO: 282 | SEQ ID NO: 301 |
| PV RdRP-NC_033770 | SEQ ID NO: 81 | SEQ ID NO: 283 | SEQ ID NO: 302 |
| | or 376 | | |
| PV RdRP-NC_034159 | SEQ ID NO: 82 | SEQ ID NO: 284 | SEQ ID NO: 303 |
| PV RdRP-NC_034513 | SEQ ID NO: 83 | SEQ ID NO: 285 | SEQ ID NO: 304 |
| PV RdRP-NC_037095 | SEQ ID NO: 84 | SEQ ID NO: 286 | SEQ ID NO: 305 |
| | or 372 | | |
| PV RdRP-NC_038824 | SEQ ID NO: 85 | SEQ ID NO: 287 | SEQ ID NO: 306 |
| PV RdRP-NC_038846 | SEQ ID NO: 86 | SEQ ID NO: 288 | SEQ ID NO: 307 |
| PV RdRP-NC_040483 | SEQ ID NO: 87 | SEQ ID NO: 289 | SEQ ID NO: 308 |
| PV RdRP-NC_043394 | SEQ ID NO: 88 | SEQ ID NO: 290 | SEQ ID NO: 309 |
| PV RdRP-JX971981 | SEQ ID NO: 382 | SEQ ID NO: 384 | SEQ ID NO: 385 |
| PV RdRP from Zea mays | SEQ ID NO: 386 | SEQ ID NO: 390 | SEQ ID NO: 388 |
| Wu312 | | | or 389 |

TABLE 9
Partitiviral Capsid Genome 5′ and 3′ Replication Element Pairs

| | PV Capsid | PV Capsid |
|---|---|---|
| | Protein | Protein |
| | Genome-5′ | Genome-3′ |
| | Replication | Replication |
| Partitivirus | Element | Element |
| Genomes | SEQ ID NO | SEQ ID NO |
| NC_006276 | 2 | 36 |
| NC_007242 | 3 | 37 |
| NC_008190 | 4 | 38 |
| NC_009520 | 5 | 39 |
| NC_009521 | 6 | 40 |
| NC_010344 | 7 | 41 |
| NC_010345 | 8 | 42 |
| NC_010347 | 9 | 43 |
| NC_010348 | 10 | 44 |
| NC_011557 | 11 | 45 |
| NC_011706 | 12 | 46 |
| NC_013110 | 13 | 47 |
| NC_015495 | 14 | 48 |
| NC_017988 | 15 | 49 |
| NC_021095 | 16 | 50 |
| NC_021097 | 17 | 51 |
| NC_021099 | 18 | 52 |
| NC_021148 | 19 | 53 |
| NC_022615 | 20 | 54 |
| NC_022617 | 21 | 55 |
| NC_030245 | 22 | 56 |
| NC_030890 | 23 | 57 |
| NC_031130 | 24 | 58 |
| NC_033771 | 25 or 378 | 59 or 379 |
| NC_034167 | 26 | 60 |
| NC_034523 | 27 | 61 |
| NC_037096 | 28 or 374 | 62 or 375 |
| NC_038823 | 29 | 63 |
| NC_038838 | 30 | 64 |
| NC_038845 | 31 | 65 |
| NC_038847 | 32 | 66 |
| NC_040456 | 33 | 67 |
| NC_040484 | 34 | 68 |
| JX971981 | 380 | 381 |

TABLE 10
Partitiviral RdRP Genome 5′ and 3′
Replication Element Pairs and RdRP

| | | PV RdRP | PV RdRP | |
|---|---|---|---|---|
| | | Genome-5′ | Genome-3′ | |
| | | Replication | Replication | PV RdRP |
| | | Element | Element | Protein |
| Row | Partitivirus | SEQ ID NO | SEQ ID NO | SEQ ID NO |
| 1 | NC_006275 | 70 | 90 | 272 |
| 2 | NC_007241 | 71 | 91 | 273 |
| 3 | NC_009519 | 72 | 92 | 274 |
| 4 | NC_010343 | 73 | 93 | 275 |
| 5 | NC_010346 | 74 | 94 | 276 |
| 6 | NC_011556 | 75 | 95 | 277 |
| 7 | NC_011705 | 76 | 96 | 278 |
| 8 | NC_015494 | 77 | 97 | 279 |
| 9 | NC_017989 | 78 | 98 | 280 |
| 10 | NC_022614 | 79 | 99 | 281 |
| 11 | NC_030243 | 80 | 100 | 282 |
| 12 | NC_033770 | 81 or 376 | 101 or 377 | 283 |
| 13 | NC_034159 | 82 | 102 | 284 |
| 14 | NC_034513 | 83 | 103 | 285 |
| 15 | NC_037095 | 84 or 372 | 104 or 373 | 286 |
| 16 | NC_038824 | 85 | 105 | 287 |
| 17 | NC_038846 | 86 | 106 | 288 |
| 18 | NC_040483 | 87 | 107 | 289 |
| 19 | NC_043394 | 88 | 108 | 290 |
| 20 | JX971981 | 382 | 383 | 384 |
| 21 | PV from Zea | 386 | 387 | 390 |
| | mays Wu312 | | | |

### Examples

### Example 1. Replication of PCV1 and SCV1 in Nicotiana benthamiana plants

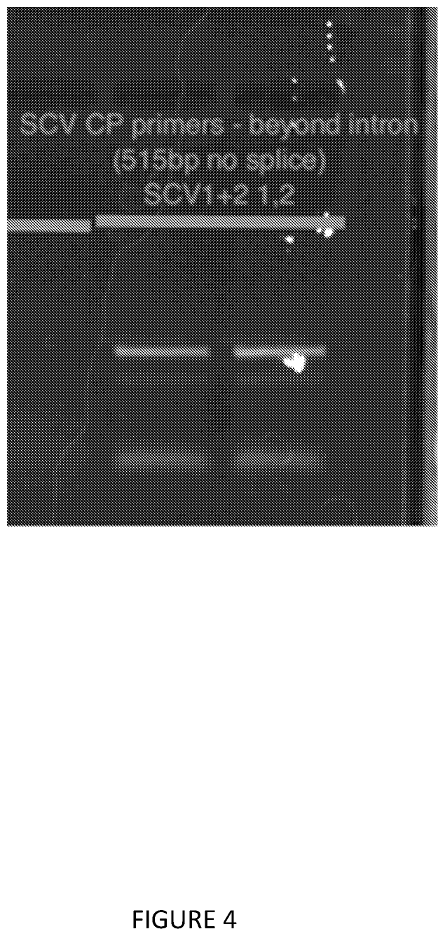

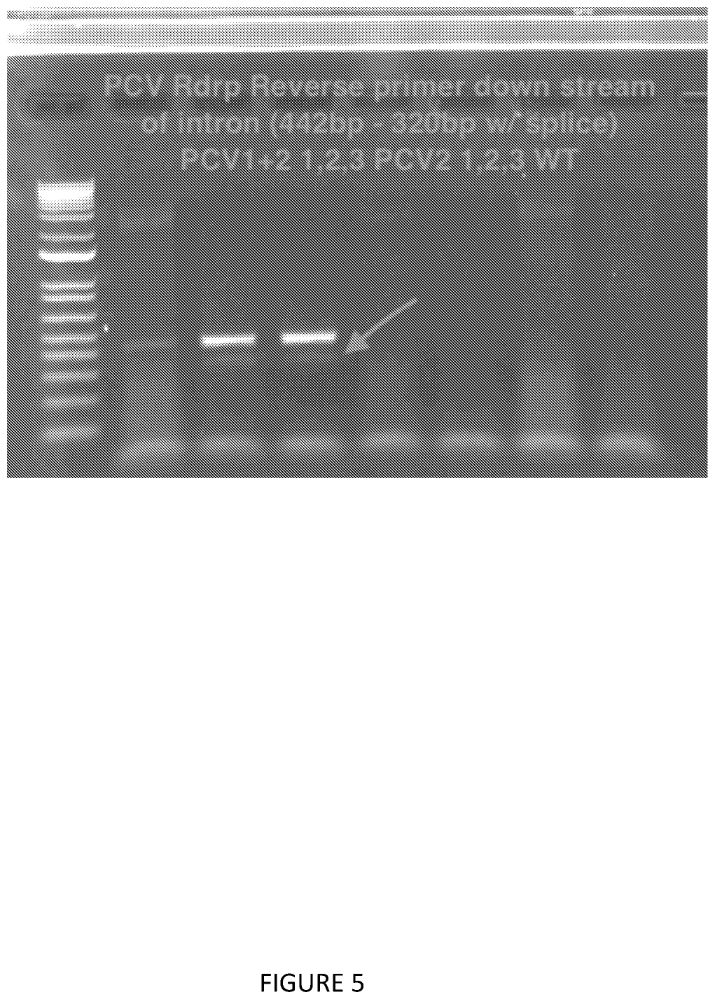

T-DNAs that encoded expression cassettes for intron containing RDRP and CP genomes of Pepper cryptic virus 1 (PCV1) and Spinach cryptic virus 1 (SCV1) were constructed. The vector comprising the T-DNA encoding the PCV1 RDRP genome is shown in FIG. 8 and the sequence of the vector is provided in SEQ ID NO: 255 (Table 11). The vector comprising the T-DNA encoding the PCV1 CP genome is shown in FIG. 9 and the sequence of the vector is provided in SEQ ID NO: 256 (Table 12). The vector comprising the T-DNA encoding the SCV1 RDRP genome is shown in FIG. 10 and the sequence of the vector is provided in SEQ ID NO: 257 (Table 13). The vector comprising the T-DNA encoding the SCV1 CP genome is shown in FIG. 12 and the sequence of the vector is provided in SEQ ID NO: 259 (Table 14). These T-DNA-containing vectors were transformed into Agrobacterium. Strains that had T-DNAs encoding the RDRP and CP of a particular partitivirus were infiltrated into Nicotiana benthamiana leaves. After six days, RNA was collected from the site of infiltration and negative-strand-specific primers were used to generate cDNA. This cDNA was then assayed using RT-PCR for the presence of the correctly spliced RNA genomes, which would only be produced if the transcribed RNA had been replicated by the delivered viral RDRP, indicating that the constructs were able to recapitulate viral replication in vivo. Results of the experiment are shown in FIGS. 4 and 5. Primers which hybridize to the negative strand of the PCV1 and SCV1 genomes each produced a product indicative of viral replication mediated by the RDRP.

TABLE 11

| | Nucleotide position |
|---|---|
| Genetic element | in SEQ ID NO: 255 |
| 35S promoter | 7960 to 8342 |
| Hammerhead ribozyme | 8353 to 8389 |
| PCV1 RDRP 5′UTR | 8390 to 8481 |
| | (FIG. 13A) |
| | (SEQ ID NO: 372) |
| PCV1 RDRP coding | 8483 to 1193 |
| region with intron | (encoding |
| | SEQ ID NO: 286) |
| Intron | 912 to 1035 |
| PCV1 RDRP 3′UTR | 1198 to 1228 |
| | (FIG. 13B) |
| | (SEQ ID NO: 373) |
| HDV ribozyme | 1229 to 1296 |
| 35S terminator | 1301 to 1491 |

TABLE 12

| | Nucleotide position |
|---|---|
| Genetic element | in SEQ ID NO: 256 |
| 35S promoter | 73 to 455 |
| Hammerhead ribozyme | 466 to 502 |
| PCV1 CP 5′UTR | 503 to 597 |
| | (FIG. 13C) |
| | (SEQ ID NO: 374) |
| PCV1 CP coding | 598 to 1960 |
| region with intron | (encoding |
| | SEQ ID NO: 336) |
| Intron | 1592 to 1715 |
| PCV1 CP 3′UTR | 1961 to 2138 |
| | (FIG. 13D) |
| | (SEQ ID NO: 375) |
| HDV ribozyme | 2139 to 2206 |
| 35S terminator | 2211 to 2401 |

TABLE 13

| | Nucleotide position |
|---|---|
| Genetic element | in SEQ ID NO: 257 |
| 35S promoter | 73 to 455 |
| Hammerhead ribozyme | 466 to 502 |
| SCV1 RDRP 5′UTR | 503 to 582 |
| | (FIG. 14A) |
| | (SEQ ID NO: 376) |
| SCV1 RDRP coding | 583 to 2557 |
| region with intron | (encoding |
| | SEQ ID NO: 283) |
| Intron | 2258 to 2381 |
| SCV1 RDRP 3′UTR | 2558 to 2592 |
| | (FIG. 14B) |
| | (SEQ ID NO: 377) |
| HDV ribozyme | 2593 to 2660 |
| 35S terminator | 2665 to 2855 |

TABLE 14

| | Nucleotide position |
|---|---|
| Genetic element | in SEQ ID NO: 259 |
| 35S promoter | 73 to 455 |
| Hammerhead ribozyme | 466 to 502 |
| SCV1 CP 5′UTR | 503 to 612 |
| | (FIG. 14C) |
| | (SEQ ID NO: 378) |
| SCV1 CP coding | 613 to 2203 |
| region with intron | (encoding |
| | SEQ ID NO: 333) |
| Intron | 1459 to 1582 |
| SCV1 CP 3′UTR | 2204 to 2388 |
| | (FIG. 14D) |
| | (SEQ ID NO: 379) |
| HDV ribozyme | 2389 to 2456 |
| 35S terminator | 2461 to 2651 |

### Example 2. Systemic Movement of SCV1 in Spinach Plants with Support of Movement Protein

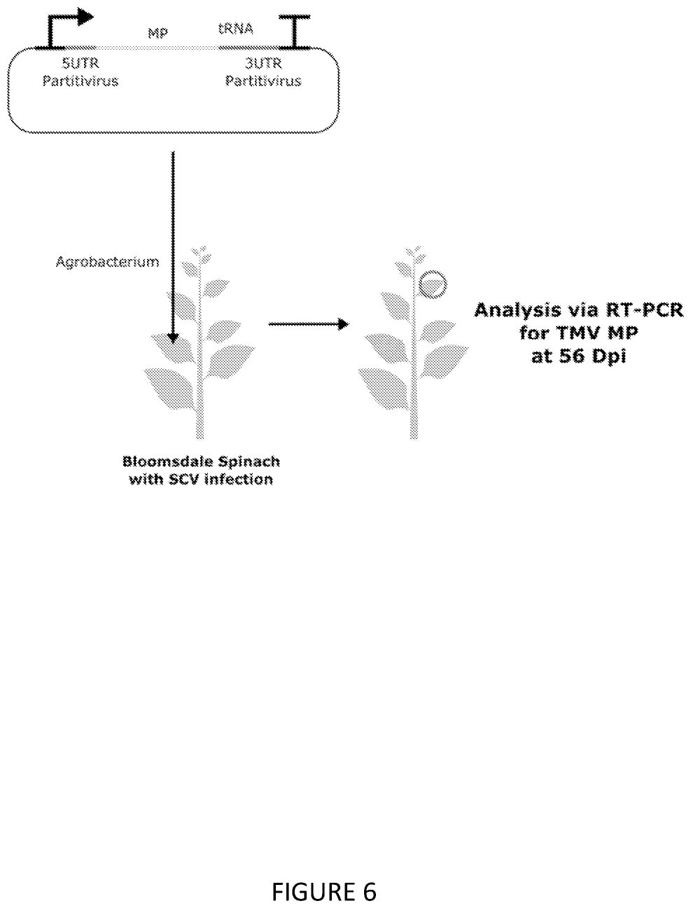

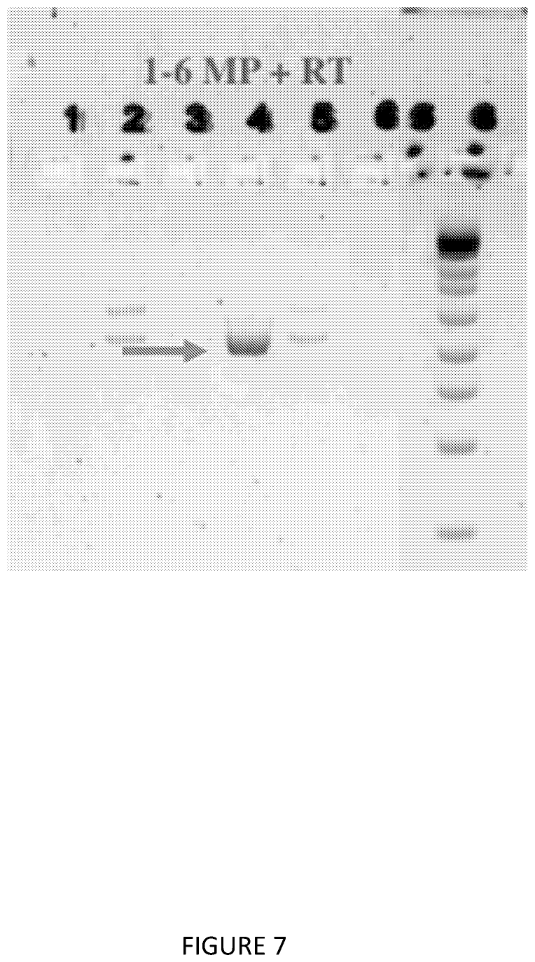

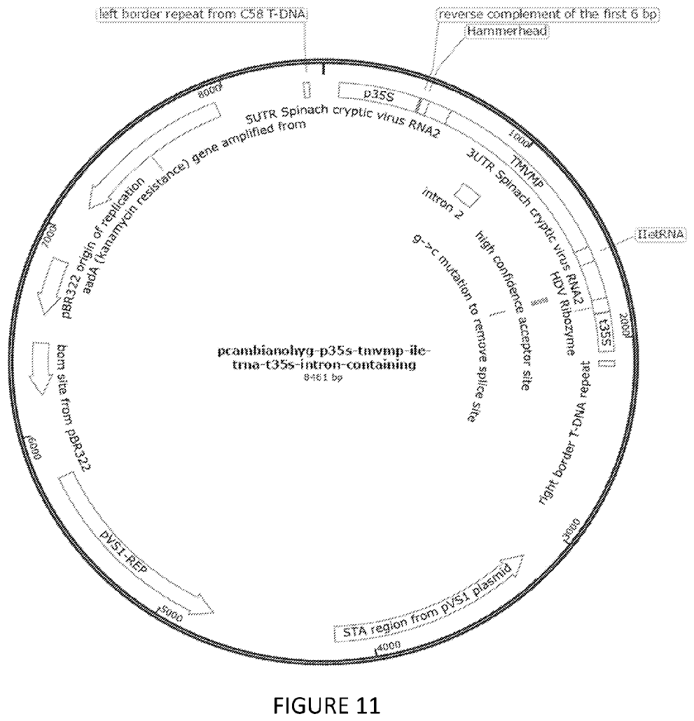

The experiment illustrated in FIG. 6 was conducted as follows. A T-DNA encoding an expression cassette for a partitiviral commensal satellite RNA (“comsat”) for the spinach cryptic virus 1 (SCV1) with a cargo RNA encoding a TMV movement protein (MP) and a tRNA-like element was constructed. The vector containing this T-DNA is shown in FIG. 11 and the sequence of the vector is provided as SEQ ID NO: 258 (Table 15). This T-DNA was transformed into Agrobacterium. The resulting strain was used to infiltrate a leaf on Bloomsdale spinach plants which were infected with SCV1 and produced SCV1 RDRP. After 58 days RNA was collected from systemic, uninfiltrated leaves, and RT-PCR for the TMV MP cargo was performed. Mobility and persistence of the SCV1 comsat was observed in several plants, as shown in FIG. 7. This result was replicated in a second set of experiments.

TABLE 15

| | Nucleotide position |
|---|---|
| Genetic element | in SEQ ID NO: 258 |
| 35S promoter | 73 to 455 |
| Hammerhead | 466 to 502 |
| SCV1 CP 5′UTR | 503 to 612 |
| | (SEQ ID NO: 378) |
| TMV MP coding | 613 to 1543 |
| region with intron | (encoding |
| | SEQ ID NO: 159) |
| Intron | 872 to 995 |
| SCV1 CP 3′UTR | 1618 to 1802 |
| | (SEQ ID NO: 379) |
| HDV Ribozyme | 1803 to 1870 |
| 35S terminator | 1875 to 2065 |

### Example 3. Vertical Transmission of a Cargo RNA in Plants

This example demonstrates how a partitiviral commensal satellite RNA can be used to generate plants with the satellite RNA without necessitating transgenesis.

Nicotiana benthamiana plants comprising the SCV1 or PCV1 genomes set forth in Example 1, Spinach plants comprising the SCV comsat set forth in Example 2, or plants (e.g., Nicotiana benthamiana, spinach, pepper, or tomato) comprising other partitiviral commensal satellite RNAs (“comsats”) provided herein (e.g., comsats comprising scoreable or selectable markers) are generated. Systemic mobility and persistence of these molecules is demonstrated across these plants using RT-PCR in systemic tissues. The plants are then grown to adulthood and their fruits were harvested. The tissue of the plants (e.g., pericarp of the fruits) are screened using RT-PCR, the selectable marker, or the scoreable marker for the presence of delivered partitiviral comsat and assayed fruit containing the comsat are identified.

Seeds are collected from mature fruit of these plants, vernalized where necessary, and then planted in soil. Cotyledons or other tissues from these seedlings are then collected and RNA was extracted independently. A pooled screening strategy can be used where RNA from seedlings is combined, and RT-PCR is used to look for presence of the delivered comsats or encoded products (e.g., cargo RNAs or proteins) in these tissues. A positive signal is observed in pooled groups. Each of the individuals in the positive pooled groups are assayed and one or more positive individuals per pool are identified. Thus, results indicating a vertical transmission of the partitiviral comsat are thus obtained.

The plants with successful vertical transmission are then grown to adulthood. Here the cargo RNA in the comsat or protein encoded by the comsat are expected to be expressed. Such expression can be confirmed by PT-PCR assays (for RNA), protein assays (immunologic and/or for enzymatic activity where applicable), and/or phenotypic assays (e.g., for traits conferred by the encoded RNA or protein).

### Example 4. Replication of PCV1 in Bell Pepper Plants

Agrobacterium strains having the T-DNAs encoding the RDRP and CP genomes of PCV1 set forth in Example 1 (SEQ ID NOs: 255 and 256) were infiltrated into bell pepper (Capsicum annuum) leaves. After five days, RNA was collected from the site of infiltration. cDNA was generated and assayed using RT-PCR for the presence of the correctly spliced RNA. Primers produced a product corresponding to amplification of a cDNA synthesized from both the (+) and (−) strand of spliced RNA which was independent of the presence of the T-DNA encoding the PCV1 RDRP.

### Example 5. Systemic Movement of PCV1 in Nicotiana benthamiana Plants

Agrobacterium strains having the T-DNAs encoding the RDRP and CP genomes of PCV1 set forth in Example 1 (SEQ ID NOs: 255 and 256) were infiltrated into Nicotiana benthamiana leaves. After 28 days, RNA was collected from tissue at the site of infiltration, from surrounding tissue, and from apical leaf tissue to detect systemic movement. cDNA was generated from the RNA and assayed using RT-PCR for the presence of the spliced RNA. The spliced RNA was detected in local leaf tissues as well as the apical leaf tissue, indicating that at least PCV1 RNA moved systemically in Nicotiana benthamiana despite lacking a movement protein. Sequencing of the apical leaf tissue PCR product confirmed that the signal was from PCV1 RNA.

### Example 6. Replication and Systemic Movement of SCV1 in Nicotiana benthamiana Plants

Agrobacterium strains having the T-DNAs encoding the RDRP and CP genomes of SCV1 set forth in Example 1 (SEQ ID NOs: 257 and 259) and the T-DNA encoding the SCV1 comsat with a cargo RNA encoding a TMV MP set forth in Example 2 (SEQ ID NO: 258) were infiltrated into Nicotiana benthamiana leaves.

After 5 days, RNA was collected from infiltrated leaves and distal leaves (above the infiltrated leaves). cDNA was generated from the RNA and assayed using RT-PCR for the presence of the spliced RNA. The spliced RNA was detected in both positive and negative sense RNA from infiltrated leaf tissue. No movement to distal leaves was detected at 5 days post inoculation.

After 14 days, RNA was collected from infiltrated leaves and distal leaves. cDNA was generated from the RNA and assayed using RT-PCR for the presence of the spliced RNA. The spliced RNA was detected in both positive and negative sense RNA from infiltrated leaf tissue, indicating persistence of SCV1 RNA in Nicotiana benthamiana (a non-host plant) for up to two weeks.

After 28 days, RNA was collected from infiltrated leaves, distal leaves, and apical leaves. cDNA was generated from the RNA and assayed using RT-PCR for the presence of the spliced RNA. The spliced RNA was detected in both positive and negative sense RNA from infiltrated leaf tissue as expected. The spliced RNA was also detected in distal leaf and apical leaf tissues, indicating that SCV1 RNA moved systemically in Nicotiana benthamiana with support of the movement protein. Sequencing of the distal and apical leaf tissue PCR products confirmed that the signals were from SCV1 RNA.

At 5, 14, and 28 dpi, the presence of the positive and negative stranded SCV1 spliced RNA was independent of the presence of the SCV1 RdRP encoded by the T-DNA.

### OTHER EMBODIMENTS

Although the foregoing disclosure has been described in some detail by way of illustration and example for purposes of clarity of understanding, the descriptions and examples should not be construed as limiting the scope of the disclosure.

Other embodiments are within the claims.

1.-52. (canceled)

53. A method of manufacturing a synthetic partitiviral satellite particle, comprising combining a recombinant single-stranded RNA (ssRNA) molecule with a viral capsid protein, wherein the recombinant ssRNA molecule comprises from 5′ terminus to 3′ terminus:
(a) a 5′ RNA replication element recognized by a partitiviral RNA-dependent RNA polymerase (RDRP), wherein the 5′ RNA replication element comprises at least a segment of the 5′ untranslated region (UTR) of a partitiviral genome or a variant thereof having one or more nucleotide substitutions, insertions, and/or deletions;
(b) a cargo RNA molecule;
(c) a 3′ RNA replication element recognized by the RDRP, wherein the 3′ RNA replication element comprises at least a segment of the 3′ untranslated region (UTR) of the partitiviral genome or a variant thereof having one or more nucleotide substitutions, insertions, and/or deletions;
wherein the 5′ RNA replication element, the cargo RNA molecule, and the 3′ RNA replication element are operably linked and wherein the cargo RNA molecule is heterologous to the 5′ RNA replication element and the 3′ RNA replication element; and an encapsidation recognition element (ERE), wherein the ERE provides for encapsidation of the ssRNA by the viral capsid protein.

54. The method of claim 53, wherein the recombinant ssRNA molecule is combined with the viral capsid protein in a vessel, optionally wherein the method further comprises isolating the synthetic partitiviral satellite particle from uncombined RNA and/or viral capsid protein in the vessel.

55. The method of claim 53, wherein the combining comprises
(a) providing to a plant cell the recombinant ssRNA molecule, wherein the plant cell comprises the partitiviral RDRP protein that recognizes the 5′ RNA replication element and 3′ RNA replication element and catalyzes synthesis of a synthetic partitiviral satellite RNA from the recombinant RNA molecule and the viral capsid protein, wherein the viral capsid protein encapsidates the synthetic partitiviral satellite RNA to form a synthetic partitiviral satellite particle; and optionally
(b) isolating the synthetic partitiviral satellite particle from the plant cell, a plant comprising the plant cell, or from media in which the plant cell or the plant had been grown.

56. The method of claim 53, further comprising the step of formulating the synthetic partitiviral satellite particle wherein the formulating comprises combining the synthetic partitiviral satellite particle with a carrier, an excipient, and/or an adjuvant.

57. The method of claim 53, wherein:
(a) the 5′ RNA replication element comprises at least one RNA secondary structure adopted by an RNA molecule encoded by SEQ ID NO: 1-34, 69-87, or 88, 372, 374, 376, 378, 380, 382, or 386; and/or
(b) the 3′ RNA replication element comprises at least one RNA secondary structure adopted by an RNA molecule encoded by SEQ ID NO: 35-68, 89-107, or 108, 373, 375, 377, 379, 381, 383, or 387.

58. The method of claim 53, wherein:
(a) the 5′ RNA replication element comprises an RNA molecule encoded by SEQ ID NO: 1-34, 69-87, or 88, 372, 374, 376, 378, 380, 382, or 386; a variant thereof encoded by a DNA molecule having at least 70%, 75%, 80%, 85%, 90%, 95%, 98%, or 99% sequence identity to SEQ ID NO: 1-34, 69-87, or 88, 372, 374, 376, 378, 380, 382, or 386; or a variant thereof wherein one or more nucleotides in the RNA secondary structure are substituted with distinct nucleotides which maintain the RNA secondary structure; and/or
(b) the 3′ RNA replication element comprises an RNA molecule encoded by SEQ ID NO: 35-68, 89-107, or 108, 373, 375, 377, 379, 381, 383, or 387; a variant thereof encoded by a DNA molecule having at least 70%, 75%, 80%, 85%, 90%, 95%, 98%, or 99% sequence identity to SEQ ID NO: 35-68, 89-107, or 108, 373, 375, 377, 379, 381, 383, or 387; or a variant thereof wherein one of more base-paired residues in the RNA secondary structure are substituted with distinct nucleotides which maintain the RNA secondary structure.

59. The method of claim 58, wherein the RNA secondary structure is maintained by substituting nucleotides in the secondary structure which are not base paired with nucleotides which will not base pair and/or by substituting nucleotides in the secondary structure which are base paired with nucleotides which will base pair.

60. The method of claim 53, wherein:
(a) wherein the 5′ replicase replication element further comprises a genomic sequence of the partitivirus that is natively located 3′ to and adjacent to the 5′ UTR sequence; and/or
(b) wherein the 3′ replicase replication element further comprises a genomic sequence of the partitivirus that is natively located 5′ to and adjacent to the 3′ UTR sequence, and optionally wherein the partitiviral genome of (a) and (b) are the same.

61. The method of claim 53, wherein the ssRNA molecule further comprises at least one of: (i) a tRNA-like element, optionally wherein the tRNA-like element provides for intercellular movement of the RNA and optionally wherein the intercellular movement is mediated by a viral movement protein (MP); (ii) an RNA effecter; and/or (iii) RNA encoding a viral movement protein (MP), optionally wherein an internal ribosome entry site (IRES) is operably linked to the RNA encoding the MP.

62. The method of claim 61, wherein the tRNA-like element comprises a tRNA-like molecule from an Arabidopsis FT mRNA, or is a tRNA-like sequence selected from SEQ ID NOs: 184-231, or is a modified tRNA-like sequence that has at least 90% sequence identity to a scaffold tRNA-like sequence selected from the group consisting of SEQ ID NOs: 184-231 and that maintains the secondary structure of the scaffold tRNA-like sequence.

63. The method of claim 53, wherein the ERE is a tobacco mosaic virus (TMV) OAS.

64. The method of claim 53, wherein the cargo RNA molecule is up to about 3.2 kb in length, optionally wherein the cargo RNA molecule comprises: (a) at least one coding sequence, optionally wherein the coding sequence encodes a selectable or scoreable marker: (b) at least one non-coding sequence; or (c) both at least one coding sequence and at least one non-coding sequence.

65. The method of claim 64, wherein the cargo RNA molecule comprises at least one coding sequence, and wherein the RNA molecule further comprises an internal ribosome entry site (IRES) which is operably linked to the at least one coding sequence, optionally wherein the operably linked IRES is located 5′ and immediately adjacent to the coding sequence.

66. The method of claim 64, wherein the cargo RNA molecule comprises at least one non-coding sequence, and wherein the at least one non-coding sequence is a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ligand-responsive ribozyme (aptazyme); an RNA aptamer; or a long noncoding RNA (lncRNA).

67. The method of claim 53, further comprising: (i) an RNA comprising at least one ribozyme, optionally wherein the at least one ribozyme is located 5′ to the 5′ RNA replication element or 3′ to the 3′ RNA replication element; or (ii) an RNA molecule comprising at least one ligand-responsive ribozyme (aptazyme), optionally wherein the at least one ligand-responsive ribozyme is located 5′ to the 5′ RNA replication element or 3′ to the 3′ RNA replication element.

68. The method of claim 53, wherein: (i) the 5′ RNA replication element and the 3′ RNA replication element are obtained from the same partitiviral genome or from related partitiviral genomes having at least 85%, 90%, 95%, 98%, or 99% sequence identity to one another; (ii) the 5′ RNA replication element, the 3′ RNA replication element, and the RDRP are obtained from the same partitiviral genome or from related partitiviral genomes having at least 85%, 90%, 95%, 98%, or 99% sequence identity to one another; or (iii) wherein the 5′ RNA replication element, the 3′ RNA replication element, and/or the RDRP coding region are obtained from two partitiviral genomes wherein the members of each pair of the 5′ RNA replication elements, 3′ RNA replication elements, and/or RDRP coding region have at least 85%, 90%, 95%, 98%, or 99% sequence identity to one another.

69. The method of claim 53, wherein the recombinant RNA molecule is encapsidated by a viral capsid protein, wherein the viral capsid protein is a partitiviral capsid protein, and wherein the ssRNA comprises an ERE which provides for encapsidation of the ssRNA by the partitiviral capsid protein.

70. A synthetic partitiviral satellite particle made by the method of claim 53.

71. An agricultural formulation comprising the synthetic partitiviral satellite particle made by the method of claim 53.

72. A method of providing a synthetic partitiviral satellite particle to a plant, comprising contacting the plant with the formulation of claim 71.
